(19) United States
(12) Patent Application Publication
(10) Pub. No.: US 2024/0400990 A1
(43) Pub. Date: Dec. 5, 2024

(54) IMMUNE CELL POPULATIONS AND USES THEREOF

(71) Applicant: Marengo Therapeutics, Inc., Cambridge, MA (US)

(72) Inventors: Adrian Hayday, Orpington Kent (GB); Gurkan Guntas, Newton, MA (US); Andrew Bayliffe, London (GB); Madan Katragadda, Acton, MA (US); Pierre Vantourout, London (GB); Dulce Nombre de Maria Conde Poole, London (GB); Khiyam Hussain, Hampshire (GB); Josephine Eum, London (GB)

(21) Appl. No.: 18/654,860

(22) Filed: May 3, 2024

Related U.S. Application Data
(63) Continuation of application No. PCT/US2022/049039, filed on Nov. 4, 2022.
(60) Provisional application No. 63/276,357, filed on Nov. 5, 2021.
(60) Provisional application No. 63/356,620, filed on Jun. 29, 2022.
(60) Provisional application No. 63/369,610, filed on Jul. 27, 2022.
(60) Provisional application No. 63/369,742, filed on Jul. 28, 2022.

(51) Int. Cl. C12N 5/0783; C07K 16/28; G01N 33/50

(52) U.S. Cl. CPC C12N 5/0636; C07K 16/2809; G01N 33/5047; C12N 2501/2302; C12N 2501/2307; C12N 2501/2312; C12N 2501/2315; C12N 2501/2318; C12N 2501/2321; C12N 2501/24; C12N 2501/998

(57) ABSTRACT

Provides herein are various immune cell populations for treating conditions or diseases in a subject in need thereof. Also provided are methods of expanding T cell populations using molecules that bind to a T cell receptor beta variable region (TCRβV) for treating conditions or diseases in a subject using the same.

### RELATED APPLICATIONS

This application is a continuation of International Application No. PCT/US2022/049039 filed on Nov. 4, 2022 which claims the benefit of U.S. Provisional Patent Application No. 63/276,357 filed on Nov. 5, 2021, U.S. Provisional Patent Application No. 63/356,620 filed on Jun. 29, 2022, U.S. Provisional Patent Application No. 63/369,610 filed on Jul. 27, 2022, and U.S. Provisional Patent Application No. 63/369,742 filed on Jul. 28, 2022, each of which is incorporated herein by reference in its entirety.

### SEQUENCE LISTING

The instant application contains a Sequence listing which has been submitted electronically in XML format and is hereby incorporated by reference in its entirety. Said XML copy, created on May 3, 2024, is named 53676-747_601_SL.xml and is 1,471,982 bytes in size.

### BACKGROUND

Currently available molecules designed to redirect T cells to promote tumor cell lysis for cancer immunotherapy typically target the CD3 epsilon (CD3e; CD3ε) subunit of the T cell receptor (TCR). However, there are limitations to this approach. Previous studies have shown that, e.g., low doses of anti-CD3e monoclonal antibody (mAb) can cause T cell dysfunction and exert immunosuppressive effects. In addition, anti-CD3e mAbs bind to all T cells and thus activate a large number of T cells. Such non-physiological massive activation of T cells by these anti-CD3e mAbs can result in the production of proinflammatory cytokines such as IFN-gamma, IL-1-beta, IL-6, IL-10 and TNF-alpha, causing a “cytokine storm” known as the cytokine release syndrome (CRS), which is also associated with neurotoxicity (NT). Thus, there is a need for improved T cell receptor-binding molecules that redirect T cells for cancer immunotherapy.

### SUMMARY

Provided herein is a pharmaceutical composition comprising a population of immune cells, wherein at least 50% of the population of immune cells are central memory (CM) T cells, wherein the CM T cells are induced by binding to a molecule that binds to a T cell receptor beta variable region (TCRβV). In some embodiments, at least 60% of the population of immune cells are CM T cells. In some embodiments, at least 70% of the population of immune cells are CM T cells. In some embodiments, wherein at least 80% of the population of immune cells are CM T cells.

In some embodiments, the population of immune cells are cultured in a growth medium ex vivo. In some embodiments, the molecule binds to a germline encoded region of the TCRβV. In some embodiments, the molecule binds to a hypervariable region 4 (HV4) of the TCRβV. In some embodiments, the molecule binds to a complementarity-determining region 2 (CDR2) of the TCRβV. In some embodiments, the population of immune cells are hyper proliferative. In some embodiments, the population of immune cells are proliferative.

In some embodiments, the CM T cells are TCRβV+. In some embodiments, at least 20% of the population of immune cells are TCRβV+. In some embodiments, the TCRβV is TCRβV1, TCRβV2, TCRβV3, TCRβV4, TCRβV5, TCRβV6, TCRβV7, TCRβV8, TCRβV9, TCRβV10, TCRβV11, TCRβV12, TCRβV13, TCRβV14, TCRβV15, TCRβ16, TCRβV17, TCRβV18, TCRβV19, TCRβV20, TCRβV21, TCRβV22, TCRβV23, TCRβV24, TCRβV25, TCRβV26, TCRβV27, TCRβV28, TCRβV29 or TCRβV30. In some embodiments, the TCRβV is TCRβV2, TCRβV3-1, TCRβV4-1, TCRβV4-2, TCRβV5-1, TCRβV5-4, TCRβV5-5, TCRβV5-6, TCRβV6-1, TCRβ6-5, TCRβV6-6, TCRβV7-3, TCRβV7-6, TCRβV7-8, TCRβV9, TCRβV11-2, TCRβV19, TCRβV21-1, TCRβV24-1, TCRβV27, TCRβV28, TCRβV29-1 or TCRβV30. In some embodiments, the TCRβV is TCRβV6-5, TCRβV20-1, TCRβV12-3, TCRβV12-4 or TCRβV5-1.

In some embodiments, the CM T cells are CCR7+. In some embodiments, at least 65% of the population of immune cells are CCR7+. In some embodiments, at most 35% of the population of immune cells are CCR7−. In some embodiments, the CM T cells are CD45RA−. In some embodiments, at least 65% of the population of immune cells are CD45RA−. In some embodiments, at most 35% of the population of immune cells are CD45RA+. In some embodiments, the CM T cells are CD95+. In some embodiments, at least 50% of the population of immune cells are CD95+. In some embodiments, at most 50% of the population of immune cells are CD95−. In some embodiments, the CM T cells are CCR7+ and CD45RA−. In some embodiments, at least 50% of the population of immune cells are CCR7+ and CD45RA−. In some embodiments, at most 7.7% of the population of immune cells are CCR7− and CD45RA+. In some embodiments, the CM T cells are CD95+ and CCR7+. In some embodiments, at least 50% of the population of immune cells are CD95+ and CCR7+. In some embodiments, at most 50% of the population of immune cells are CD95− and CCR7−. In some embodiments, the CM T cells are CD95+ and CD45RA−. In some embodiments, at least 50% of the population of immune cells are CD95+ and CD45RA−. In some embodiments, at most 50% of the population of immune cells are CD95− and CD45RA+. In some embodiments, the CM T cells are CD95+. CCR7+ and CD45RA−. In some embodiments, at least 50% of the population of immune cells are CD95+, CCR7+ and CD45RA−. In some embodiments, at most 50% of the population of immune cells are CD95−, CCR7− and CD45RA+.

In some embodiments, the CM T cells are CD38+. In some embodiments, at least 65% of the population of immune cells are CD38+. In some embodiments, at most 35% of the population of immune cells are CD38−. In some embodiments, the CM T cells of the population of immune cells are CD25+. In some embodiments, at least 70% of the population of immune cells are CD25+. In some embodiments, at most 30% of the population of immune cells are CD25−. In some embodiments, the CM T cells of the population of immune cells are CD38+ and CD25+. In some embodiments, at least 65% of the population of immune cells are CD38+ and CD25+. In some embodiments, at most 20% of the population of immune cells are CD38− and CD25−.

In some embodiments, the CM T cells of the population of immune cells are PD-1+. In some embodiments, at least 50% of the population of immune cells are PD-1+. In some embodiments, at most 50% of the population of immune cells are PD-1−. In some embodiments, the CM T cells of the population of immune cells are TIM-3+. In some embodiments, at least 42% of the population of immune cells are TIM-3+. In some embodiments, at most 10% of the population of immune cells are TIM-3−. In some embodiments, the CM T cells of the population of immune cells are PD-1+ and TIM-3+. In some embodiments, at least 50% of the population of immune cells are PD-1+ and TIM-3+. In some embodiments, at most 7% of the population of immune cells are PD-1- and TIM-3−. In some embodiments, the CM T cells of the population of immune cells are IFNγ+. In some embodiments, at least 35% of the population of immune cells are IFNγ+. In some embodiments, at most 65% of the population of immune cells are IFNγ−. In some embodiments, the CM T cells of the population of immune cells are TNFα+. In some embodiments, at least 14% of the population of immune cells are TNFα+. In some embodiments, at most 86% of the population of immune cells are TNFα−. In some embodiments, the CM T cells of the population of immune cells are IFNγ+ and TNFα+. In some embodiments, at least 10% of the population of immune cells are IFNγ+ and TNFα+. In some embodiments, at most 52% of the population of immune cells are IFNγ− and TNFα−. In some embodiments, at least 10% of the population of immune cells are CD8+. In some embodiments, the CM T cells of the population of immune cells are IFNγ+. In some embodiments, at least 43% of the population of immune cells are IFNγ+. In some embodiments, at most 56% of the population of immune cells are IFNγ−. In some embodiments, the CM T cells of the population of immune cells are TNFα+. In some embodiments, at least 14% of the population of immune cells are TNFα+. In some embodiments, at most 86% of the population of immune cells are TNFα−. In some embodiments, the CM T cells of the population of immune cells are IFNγ+ and TNFα+. In some embodiments, at least 10% of the population of immune cells are IFNγ+ and TNFα+. In some embodiments, at most 52% of the population of immune cells are IFNγ− and TNFα−.

In some embodiments, the CM T cells of the population of immune cells are CD62L+. In some embodiments, at least 91% of the population of immune cells are CD62L+. In some embodiments, at most 9% of the population of immune cells are CD62L−. In some embodiments, the CM T cells of the population of immune cells are CD44+. In some embodiments, at least 88% of the population of immune cells are CD44+. In some embodiments, at most 12% of the population of immune cells are CD44−. In some embodiments, the CM T cells of the population of immune cells are CD62L+ and CD44+. In some embodiments, at least 15% of the population of immune cells are CD62L+ and CD44+. In some embodiments, at most 4% of the population of immune cells are CD62L− and CD44−. In some embodiments, at least 35% of the cells in the population of immune cells are CD8+. In some embodiments, at most 65% of the cells in the population of immune cells are CD8−. In some embodiments, at least 65% of the cells in the population of immune cells are CD4+. In some embodiments, at most 35% of the cells in the population of immune cells are CD4−. In some embodiments, the CM T cells comprise CD4+ and/or CD8+ T cells. In some embodiments, the CM T cells comprise CD4+ T cells. In some embodiments, the CM T cells comprise CD8+ T cells. In some embodiments, at least 30% of the CM T cells are CD8+. In some embodiments, at most 70% of the CM T cells are CD8−. In some embodiments, at least 50% of the CM T cells are CD4+. In some embodiments, at most 50% of the CM T cells are CD4−.

In some embodiments, at most 35% of the cells in the population of immune cells are naive T cells. In some embodiments, at most 25% of the cells in the population of immune cells are EM T cells. In some embodiments, at most 15% of the cells in the population of immune cells are TEMRA T cells. In some embodiments, the population of immune cells is derived from a biological sample from a subject. In some embodiments, the population of immune cells is derived from a peripheral blood mononuclear cell (PBMC) sample from a subject. In some embodiments, the CM T cells in the population of immune cells are derived from memory T cells in a biological sample from a subject. In some embodiments, the CM T cells in the population of immune cells are derived from effector memory (EM) T cells in a biological sample from a subject. In some embodiments, the CM T cells in the population of immune cells are derived from effector memory cells re-expressing CD45RA+ (T_{ENRA}) T cells in a biological sample from a subject. In some embodiments, the CM T cells in the population of immune cells are derived from EM T cells and T_{EMRA} T cells in a biological sample from a subject. In some embodiments, the population of cells is an expanded population of immune cells from a cell population expanded in the presence of a molecule that binds to a TCRβV. In some embodiments, the percentage of CM T cells in the population of cells is higher than the percentage of CM T cells in a population of immune cells from the cell population expanded in the presence of a molecule that binds to a CD3. In some embodiments, the percentage of EM T cells in the population of immune cells is lower than the percentage of EM T cells in a population of cells from the cell population expanded in the presence of a CD3 binder. In some embodiments, the population of immune cells is a TCRβV binder-expanded population of cells. In some embodiments, the percentage of CM T cells in the TCRβV binder-expanded population of cells is higher than the percentage of CM T cells in a CD3 binder-expanded population of cells. In some embodiments, the percentage of EM T cells in the TCRβV binder-expanded population of cells is lower than the percentage of EM T cells in a CD3 binder-expanded population of cells.

In some embodiments, the molecule further comprises a cytokine. In some embodiments, the cytokine is selected from the group consisting of interleukin-2 (IL-2), interleukin-7 (IL-7), interleukin-12 (IL-12), interleukin-15 (IL-15), interleukin-18 (IL-18), interleukin-21 (IL-21), interferon gamma and functional fragments or variants thereof.

Provided herein is a composition comprising: a population of cells, wherein at least 50% of the population of cells are CM T cells, and a molecule that binds to a T cell receptor beta variable region (TCRβV). In some embodiments, the composition further comprises culture media. In some embodiments, the composition further comprises a growth factor or a cytokine. In some embodiments, the composition is within a container. In some embodiments, the container is a flask, a dish, a tube, a bag or a well.

Provided herein is a cell culture comprising: a population of cells, wherein at least 50% of the population of cells are CM T cells, and a molecule that binds to a T cell receptor beta variable region (TCRβV).

Provided herein is a method of vaccinating a subject comprising administering to a subject a pharmaceutical composition described herein. In some embodiments, the subject has been previously administered an antigen or a polynucleotide encoding an antigen. In some embodiments, the subject has a disease or condition. In some embodiments, T cells specific to an antigen associated with the disease or condition are elicited in the subject. In some embodiments. B cells specific to an antigen associated with the disease or condition are elicited in the subject. In some embodiments, antigen-presenting cells specific to an antigen associated with the disease or condition are elicited in the subject. In some embodiments, natural killer cells targeting an antigen associated with the disease or condition are elicited in the subject. In some embodiments, macrophages targeting an antigen associated with the disease or condition are elicited in the subject. In some embodiments, neutrophils targeting an antigen associated with the disease or condition are elicited in the subject. In some embodiments, the method further comprising administering to the subject an antigen or a polynucleotide encoding an antigen after administration of the pharmaceutical composition described herein.

Provided herein is a method for producing CM T cells comprising contacting a population of T cells with a molecule that binds to TCRβV, wherein the population of T cells are induced into CM T cells. In some embodiments, the contacting is conducted ex vivo. In some embodiments, the contacting is conducted in vivo. In some embodiments, the method differentiates EM T cells to CM T cells. In some embodiments, the method differentiates T_{EMRA} T cells to CM T cells. In some embodiments, the molecule is a multispecific molecule. In some embodiments, the multispecific molecule comprises a cytokine molecule.

Provided herein is a composition comprising CM T cells, wherein the CM T cells are produced by a method described herein.

Provide herein is a method of making a population of cells comprising: contacting a population of T cells with a molecule that binds to TCRβV; culturing the population of T cells in the presence of the molecule that binds to TCRβV for a time sufficient to produce the population of cells, wherein at least 50% of the population of cells are CM T cells. In some embodiments, the molecule that binds to TCRβV is attached to a solid surface. In some embodiments, the solid surface is a bead or a plate. In some embodiments, culturing comprises culturing the population of T cells in the presence of IL-2 and/or X-VIVO culture media.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows various ways of activating T cells for therapeutic purposes. The first method is the non-physiological Pan-T cell activation using a α-CD3 monoclonal antibody. Some disadvantages of this method are development of a cytokine storm, limited efficacy in solid tumors, high potential for T cell exhaustion, and limited immune memory for tumor antigens. The second method is the semi-physiological Pan-T cell activation using co-stimulation activators (e.g. TNFSFR agonist monoclonal antibodies). Some advantages of this method are limited toxicity and the ability to induce immune memory for tumors antigens. A disadvantage of this method is the limited efficacy that has been seen in the 1^{st} generation of these antibodies. The third method, and the one discussed in this disclosure, is the physiological activation of TCRvβ clonotype-specific T_{effector} cells using monoclonal antibodies that specifically target the variable region of the β-chain of the TCR. Research into this method is based on the observation that staphylococcus superantigens (SEB) cause the proliferation of vβ8⁺ CD8⁺ T cells that potentiate anti-tumor responses in vivo. Some advantages of this method are targeted expansion of vβ⁺ T effector cell subset, low expression of exhaustion markers, superkiller phenotype, and differential cytokine release profile.

FIG. 2A and FIG. 2B depict the results from an alanine walk experiment. FIG. 2A shows the TCR variants with alanine substitutions introduced in the β chain used in the experiment (left column), the specific residues substituted with Alanine in the β chain (center column), the resulting affinity K_{D} value (right column) from binding of α-TCRvB6-5 antibody to the TCR variants. If the K_{D} value is colored blue, then the mutation at that location has no impact on α-TCRvB6-5 binding. If the K_{D} value is colored orange, then the mutation at that location has a minor impact on α-TCRvB6-5 binding. If the K_{D} value is colored red, then the mutation at that location has a major impact on α-TCRvB6-5 binding. In addition, residues in bold in the center column are positions that differ between TCRvB6-5 and TCRvB6-6 TCRs. FIG. 2B depicts a 3D structural model that shows the locations of the different mutations on the TCR β chain. The amino acid sequence at the top shows the location of the different domains on the TCR β chain: CDR1 (highlighted in green), CDR2 (highlighted in yellow), HV4 (highlighted in pink), and CDR3 (highlighted in blue). Those regions are shaded in the same color on the structural model. In addition, the model is labeled with the locations of the specific mutations used in the alanine walk. Dark blue regions mean the mutation to that amino acid has no effect on α-TCRvB6-5 binding, orange regions mean the mutation to that amino acid has a minor effect on α-TCRvB6-5 binding, and red regions mean that mutation to that amino acid has a major effect on α-TCRvB6-5 binding.

FIG. 3A and FIG. 3B depict the results for epitope mapping experiments for TCRvβ-specific monoclonal antibodies (mAb) binding to the variable region of the TCR β chain. FIG. 3A shows the results of epitope mapping experiment performed using parental, affinity-matured and commercial anti-TCRvβ6-5-specific antibodies. The binding of each TCRvβ antibody is tested against multiple mutated Jurkat 176 cell lines that have different single amino acid mutations across the variable region on the TCR β chain. The ability of the TCRvβ6-5 antibodies to bind to these different variant TCRβ are normalized to the ability of α-CD3 antibody to bind to the same variant TCRβ. PE α-hIgG+α-TCRvβ6-5 (Parental, BJM0816) is represented by white circles. PE α-hIgG+α-TCRvβ6-5 (Affinity Matured, BKM0210) is represented by gray circles, and PE α-Vβ13.1 (an antibody that binds to TCRvβ6-5) is represented by blue diamonds. FIG. 3B depicts 3D structures of the TCR and where the different amino acid mutations are located TCR β chain. The dotted lines show the location of the single amino acid mutation and lists the amino acid change that occurred. If the mutation is labeled with blue, then that mutation has no effect on the ability of the TCRβ-specific antibody to bind to the β chain. If the mutation is labeled with orange, then that mutation has an intermediate effect on the ability of the TCRvβ-specific antibody to bind to the β chain. If the mutation is labeled with red, then the mutation has a major impact on the ability of the TCRβ-specific antibody to bind to the β chain. This figure also depicts the epitope mapping of anti-TCRβV5-1, anti-TCRβV12-3/4, anti-TCRβVβ20-1 antibodies. If the mutation is labeled with black, then the cloned mutant did not express and no expression information is available.

FIG. 4A and FIG. 4B depict the ability of TCRvβ-specific antibody stimulation of T cells to induce T cell expansion. FIG. 4A shows a flow cytometry plot where T cells are cultured on plates with no antibody, α-CD3, or α-TCRvβ6-5. The cells are then stimulated with PMA/IONO/BFA. Cells are stained with α-CD3 (y-axis) or α-TCRvβ6-5 (x-axis) and the cells are gated as either T cell or TCRvβ+ T cells. FIG. 4B shows the percentage of TCRvβ⁺ cells out of total T cells for each of the stimulation conditions (unstimulated, α-CD3, α-TCRvβ6-5, α-TCRvβ12-3/4, α-TCRvβ-1, and α-TCRvβ5-1). The dots on the graph depict that the results are from repeat experiments using independent donors.

FIG. 5 depicts the ability of TCRvβ stimulation to drive uniform ‘hyper’ proliferation phenotype. Before stimulation, the T cells are stained with CellTrace Violet. Flow cytometry plots are shown for the following in vitro growth conditions: no antibody, α-CD3, αTCRvβ6-5, α-TCRvβ20-1, α-TCRvβ12-3/4, and α-TCRvβ5-1. The cells are then stained with α-CD25 (y-axis) and gated on proliferating CD4+ T cells. The more CTV dilution (shift to the left) observed the better the proliferation. TRVB stimulation appears to drive potent cell division.

FIGS. 6A-6D show the phenotypic characteristics TCRvβ⁺ T cells. FIG. 6A shows the FACS sorting of T cells that are cultured with either α-CD3 or α-TCRvβ. The cells are sorted into TCRvβ⁻ and TCRvβ⁺ cells. FIG. 6B shows an example of the expression levels of CD38, CD25, PD-1 on TCRvβ⁺ or TCRvβ⁻ cells after cultured with either α-CD3 or one of the specific TCRvβ example, α-TCRvβ6-5. The red graphs show the TCRvβ⁺ and TCRvβ⁻ cells resulting from α-CD3 growth and the blue graphs show the TCRvβ⁺ and TCRvβ⁻ cells resulting from α-TCRvβ6-5 growth. The graph on the left shows the expression levels of CD38 (y-axis) and CD25 (x-axis) and the graphs on the right shows the expression levels of PD-1 (y-axis) and TIM-3 (x-axis). FIG. 6C shows the median fluorescence intensity (MFI) of CD25 expression on the TCRvβ⁺ or TCRvβ⁻ cells cultured using different mAb (α-CD3, α-TCRvβ6-5, α-TCRvβ20-1, α-TCRvβ12-3/4, and α-TCRvβ5-1). Red dots represent TCRvβ⁺ cells and blue dots represent TCRvβ⁻ cells. FIG. 6D shows the percent of PD-1⁺ cells from the TCRvβ⁺ or TCRvβ⁻ cell populations cultured using different mAb (α-CD3, α-TCRvβ6-5, α-TCRvβ20-1, α-TCRvβ12-3/4, and α-TCRvβ5-1). Red dots represent TCRvβ⁺ cells and blue dots represent TCRvβ⁻ cells.

FIG. 7A and FIG. 7B show the function effect of TCRvβ stimulation on T cells. FIG. 7A shows the results of intracellular cytokine staining for IFNγ (y-axis) and TNFα (x-axis) of CD8⁺ T cells cultured on plates coated with either α-CD3, α-TCRvB5-6, α-TCRvB12-3/4, or α-TCRvB20-1. The cells are then either unstimulated or stimulated with PMA/IONO/BFA prior to read-out of IFNγ and TNFα expression. The presented flow cytometry plots are gated CD8⁺ T cells. FIG. 7B depicts the percentage of CD8⁺ T cells from the different stimulation conditions that produce both IFNγ and TNFα. The blue box shows the percentage of IFN-γ⁺ TNFα⁺ CD8⁺ T cells induced by α-CD3 stimulation. The red box shows the percentage of IFNγ⁺ TNFα⁺ CD8⁺ T cells induced by the various TCRvβ antibody stimulations.

FIG. 8A and FIG. 8B depict T-cell differentiation memory subsets based on the markers. CCR7 and CD45RA. FIG. 8A shows an example of the differentiation of T cells cultured with α-CD3 (top center), and T cells cultured α-TCRvB6-5 (top right). The bottom graph corresponds to the overlay of these two plots, α-CD3 (blue dots) or α-TCRvB6-5 (red dots). FIG. 8B shows the percentage of memory subset T cells that are central memory T cells or effector memory T cells for the following stimulation conditions: unstimulated (black dots), α-CD3 (blue dots), α-TCRvβ6-5 (red dots), α-TCRvβ20-1 (orange), α-TCRvβ12-3/4 (yellow), or α-TCRvβ5-1 (gray dots).

FIG. 9A and FIG. 9B depict the ability of the TCRvβ to drive to central memory phenotype from all memory pools. FIG. 9A shows how total T cells-naïve t cells, central memory T cells, effector memory T cells, and TEMRA T cells are sorted and cultured under one of the following conditions: no antibody (top row), α-TCRv6-5 (middle row), or α-CD3 (bottom row). After stimulation, the cells are stained with CCR7 (y-axis) and CD45RA (x-axis). Red boxes and arrows are included to highlight key phenotypic changes in effector memory and TEMRA T cell populations induced by TCRvβ stimulation. FIG. 9B shows the percentage of central memory cells achieved following subsequent stimulation of each of the sorted T cell memory subsets: unstimulated (black and white striped bars), α-TCRvB6-5 (red bars), or α-CD3 (blue bars). The black dots represent repeats from 2 experiments using 6 unique donors.

FIGS. 10A-10E depicts the workflow and analysis for Transcriptomic experiments after TCRvβ stimulation. FIG. 10A depicts the workflow for obtaining T cells from human blood samples, stimulation with either α-CD3 or α-TCRvβ, and sequencing the isolated mRNA via single-cell droplet RNA sequencing technology. FIG. 10B show UMAP (left) and tSNE (right) models of the gene expression for the unstimulated T cells (blue dots), α-CD3 T cells (orange dots), and α-TCRvβ6-5 T cells (green dots). The UMAP model additionally clusters the genes into expression by CD4⁺ or CD8⁺ T cells. FIG. 10C shows the differential gene expression of α-TCRvβ6-5 stimulated T cells compared to α-CD3 stimulated T cells via a Volcano plot. Down regulated genes are represented by dots left of 0.0 on the x-axis and upregulated genes are represented by dots right of the 0.0 on the x-axis. Black dots represent genes that are not significantly downregulated or upregulated in α-TCRvβ6-5 T cells compared to α-CD3 T cells. Red dots represent genes that are significantly downregulated or upregulated in α-TCRvβ6-5 T cells compared to α-CD3 T cells. FIG. 10D lists some of the upregulated and downregulated genes in TCRvβ T cells along with the genes' associated fold change compared to α-CD3 T cells and the associated p-values, and the gene's biological function. Gene names next to the red symbol indicates that the genes are upregulated in TCRvβ cells compared to α-CD3 cells; gene names next to the blue symbol indicates that the genes are downregulated in TCRvβ cells compared to α-CD3 cells. FIG. 10E compares the expression levels of two genes, TCF7 and IRF4, between α-TCRvB6-5 and α-CD3 cells. Darker clusters on the graph show that more cells express that specific gene compared to lighter clusters.

FIGS. 11A-11D depict a series of in vivo experiments to validate the previously presented in vitro findings. FIG. 1A outlines the study design. Groups of 10 mice are intraperitoneally administered 1 mg/kg of one of the following treatments: PBS, α-CD3, α-vB8-1 (α-mouse TCRvβ), or α-vB8-1/IL-2 (fusion molecule that contains on anti-vβ Fc arm and one IL-2 Fc arm). 7 days after administration, blood samples are collected to evaluate the in vivo activation dynamics. 14 days after administration, biological samples are collected to evaluate the long-term consequences of Vβ-specific activation. FIG. 11B shows a PCA analysis of lymph nodes collected 14 days after treatment administration. Blue dots represent responses from the α-CD3 treatment group, red dots represent the α-vB8-1/IL-2 treatment group, gray dots represent the PBS treatment group, and yellow dots represent the α-vB8-1 treatment group. FIG. 11C shows the percentage of antigen experienced CD8⁺ T cells (left graph) and the percentage of central memory CD8⁺ (right graph) that are induced in each treatment group. The blue bars represent the α-CD3 treatment group, the red bars represent the α-vB8-1/IL-2 treatment group, the gray bars represent the PBS treatment group, and the yellow bars represent the α-vB8-1 treatment group. FIG. 11D shows the number of CD8⁺ T cells isolated from the spleen collected 14 days after treatment administration. The blue bar represent the α-CD3 treatment group, the pink bar represent the RSV/IL-2 treatment group, the red bar represent the α-vB8-1/IL-2 fusion molecule (each Fc arm contains a vβ domain and an attached hIL-2, 2×2 bispecific) treatment group, the yellow bar represents the α-vB8-1 treatment group, and the orange bar represents the α-vB8-1/IL-2 fusion molecule (one Fc vβ arm and one Fc IL-2 arm, 1×1 bispecific) treatment group.

FIG. 12 shows the percentage of CD8⁺ T cells out of total T cells induced by stimulating T cells for 4 weeks in vitro. The blue bar represents T cells cultured with α-CD3, the orange bar represents T cells cultured with α-vB6-5, the red bar represents T cells cultured with α-vB20-1, and the dark red bar cultured T cells stimulated with α-vB6/hIL2 fusion (2×2 bispecific).

FIG. 13 depicts the differentiation of CD8⁺ T cells induced in mice from one of the following treatment groups: α-CD3/IL-2 (one Fc arm contains a CD3 domain and the other Fc contains a fused IL-2 molecule, 1×1 bispecific), α-vB/IL-2 fusion molecule (1×1 bispecific), α-vB/CD20 (each Fc arm contains a vβ domain and a CD20 domain, 2×2 bispecific), or PBS, CD8⁺ T cells are stained with α-CD44 (y-axis) and α-CD62L (x-axis) and the phenotypes of the CD8⁺ are evaluated at two time points: 7 days after treatment administration (top row) and 14 days after treatment administration (bottom row). Each flow cytometry plot is gated on effector memory T cell phenotype (CD44⁺ CD62L⁻), central memory T cell phenotype (CD44⁺ CD62L⁺), and naive T cell memory phenotype (CD44⁻ CD62L⁺).

FIG. 14A and FIG. 14B shows the alignment of the Antibody A source mouse VH and VL framework 1, CDR 1, framework 2, CDR 2, framework 3, CDR3, and framework 4 regions with their respective humanized sequences. Kabat CDRs are shown in bold, Chothia CDRs are shown in italics, and combined CDRs are shown in boxes. The framework positions that were back mutated are double underlined. FIG. 14A shows VH sequences for murine Antibody A (SEQ ID NO: 1) and humanized Antibody A-H (SEQ ID NO: 9). FIG. 14B shows VL sequences for murine Antibody A (SEQ ID NO: 2) and humanized Antibody A-H (SEQ ID NO: 10 and SEQ ID NO: 11).

FIG. 15 depicts the phylogenetic tree of TCRBV gene family and subfamilies with corresponding antibodies mapped. Subfamily identities are as follows: Subfamily A: TCRβ V6; Subfamily B: TCRβ V10; Subfamily C: TCRβ V12; Subfamily D: TCRβ V5; Subfamily E: TCRβ V7; Subfamily F: TCRβ V11; Subfamily G: TCRβ V14; Subfamily H: TCRβ V16; Subfamily L: TCRβ V18; Subfamily J: TCRβ V9; Subfamily K: TCRβ V13; Subfamily L: TCRβ V4; Subfamily M: TCRβ V3; Subfamily N: TCRβ V2; Subfamily O: TCRβ V15; Subfamily P: TCRβ V30; Subfamily Q: TCRβ V19; Subfamily R: TCRβ V27; Subfamily S: TCRβ V28; Subfamily T: TCRβ V24; Subfamily U: TCRβ V20: Subfamily V: TCRβ V25; and Subfamily W: TCRβ V29 subfamily. Subfamily members are described in detail herein in the Section titled “TCR beta V (TCRβV)”.

FIG. 16A and FIG. 16B show the structure and sequence of eight TCRβV proteins from seven different subfamilies: TCRβV6 subfamily (TCRβV6-5 and TCRβV6-4 are shown), TCRβV28 subfamily, TCRβV19 subfamily, TCRβV9 subfamily, TCRβV5 subfamily, TCRβV20 subfamily and TCRβV12 subfamily. FIG. 16A shows the structural alignment of the different TCRβV proteins. The circled area represents the outward facing region comprising the proposed binding site for the anti-TCRβV antibodies as described herein. FIG. 16B shows the amino acid sequence alignment of the proteins shown in FIG. 16A (SEQ ID NOS 3449-3456, respectively, in order of appearance). The various TCRβV proteins (from 7 different TCRβV subfamilies) have diverse sequences but share a conserved (similar) structure and function.

FIG. 17 depicts the alignment of TCRβV amino acid sequences (SEQ ID NOS 3457-3516, respectively, in order of appearance). The alignment of TCRβV amino acid sequences underscores the diversity of TCR sequences. In particular, the TCRvβ sequences from different subfamilies are considerably different from each other.

FIG. 18A and FIG. 18B shows the alignment of the Antibody B source mouse VH and VL framework 1, CDR 1, framework 2, CDR 2, framework 3, CDR3, and framework 4 regions with their respective humanized sequences. Kabat CDRs are shown in bold. Chothia CDRs are shown in italics, and combined CDRs are shown in boxes. The framework positions that were back mutated are double underlined. FIG. 18A shows the VH sequence for murine Antibody B (SEQ ID NO: 15) and humanized VH sequences B-H.1A to B-H.1C (SEQ ID NOs: 23-25). FIG. 18B shows the VL sequence for murine Antibody B (SEQ ID NO: 16) and humanized VL sequences B-H.1D to B-H.1H (SEQ ID NOs: 26-30).

FIGS. 19A-19C show human CD3+ T cells activated by anti-TCRβVβ13.1 antibody (A-H.1) for 6-days. Human CD3+ T cells were isolated using magnetic-bead separation (negative selection) and activated with immobilized (plate-coated) anti-TCRβ13.1 (A-H.1) or anti-CD3ε (OKT3) antibodies at 100 nM for 6 days. FIG. 19A shows two scatter plots (left: activated with OKT3; and right: activated with A-H.1) of expanded T cells assessed for TCR Vβ13.1 surface expression using anti-TCR Vβ13.1 (A-H.1) followed by a secondary fluorochrome-conjugated antibody for flow cytometry analysis. FIG. 19B shows percentage (%) of TCR Vβ13.1 positive T cells activated by anti-TCR Vβ13.1 (A-H.1) or anti-CD3e (OKT3) plotted against total T cells (CD3+). FIG. 19C shows relative cell count acquired by counting the number of events in each T cell subset gate (CD3 or TCR Vβ13.1) for 20 seconds at a constant rate of 60 μl/min. Data shown as mean value from 3 donors.

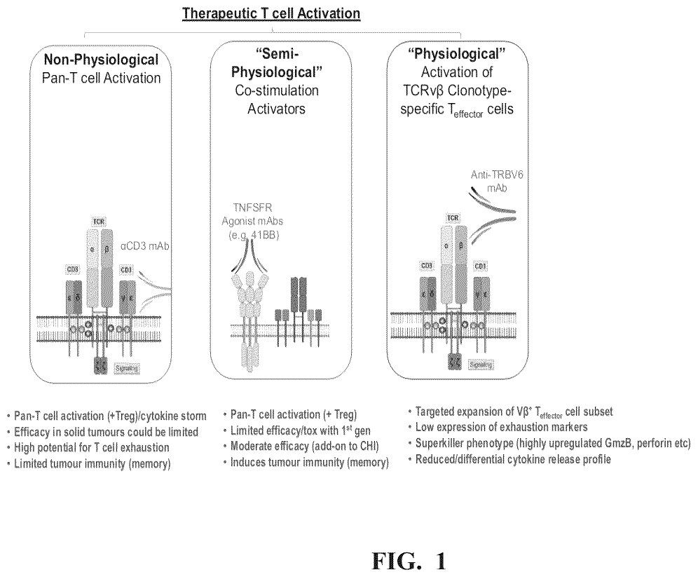

### DETAILED DESCRIPTION

### Definition

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a,” “an,” and “the” include plural referents unless the content clearly dictates otherwise. The use of the words “a” or “an” when used in conjunction with the term “comprising” herein may mean “one,” but it is also consistent with the meaning of “one or more.” “at least one,” and “one or more than one.”

It should also be noted that the term “or” is generally employed in its sense including “and/or” unless the content clearly dictates otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

The term “about” when referring to a measurable value such as an amount, a temporal duration, and the like, is meant to encompass variations of 20% or in some instances ±10%, or in some instances ±5%, or in some instances ±1%, or in some instances ±0.1% from the specified value, as such variations are appropriate to perform the disclosed methods. As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

The term “acquire” or “acquiring” as the terms are used herein, refer to obtaining possession of a physical entity (e.g., a sample, a polypeptide, a nucleic acid, or a sequence), or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Directly acquiring a value includes performing a process that includes a physical change in a sample or another substance, e.g., performing an analytical process which includes a physical change in a substance, e.g., a sample.

“Antibody molecule” as used herein refers to a protein, e.g., an immunoglobulin chain or fragment thereof, comprising at least one immunoglobulin variable domain structure and/or sequence. An antibody molecule encompasses antibodies (e.g., full-length antibodies) and antibody fragments. In some embodiments, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab. Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain. Fab, Fab′, and F(ab′)₂ fragments, and single chain variable fragments (scFvs). In some embodiments, the antibody molecule is an antibody mimetic. In some embodiments, the antibody molecule is, or comprises, an antibody-like framework or scaffold, such as, fibronectins, ankyrin repeats (e.g., designed ankyrin repeat proteins (DARPins)), avimers, affibody affinity ligands, anticalins, or affilin molecules.

The term “human-like antibody molecule” as used herein refers to a humanized antibody molecule, human antibody molecule or an antibody molecule having at least 95% sequence identity with a non-murine germline framework region, e.g., FR1, FR2. FR3 and/or FR4. In some embodiments, the human-like antibody molecule comprises a framework region having at least 95% sequence identity to a human germline framework region, e.g., a FR1, FR2, FR3 and/or FR4 of a human germline framework region. In some embodiments, the human-like antibody molecule is a recombinant antibody. In some embodiments, the human-like antibody molecule is a humanized antibody molecule. In some embodiments, the human-like antibody molecule is human antibody molecule. In some embodiments, the human-like antibody molecule is a phage display or a yeast display antibody molecule. In some embodiments, the human-like antibody molecule is a chimeric antibody molecule. In some embodiments, the human-like antibody molecule is a CDR grafted antibody molecule.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site,” or “binding portion” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions.” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia. C. et al. (1987) J. Mol. Biol. 19:901-917. Each variable chain (e.g., variable heavy chain and variable light chain) is typically made up of three CDRs and four FRs, arranged from amino-terminus to carboxy-terminus in the amino acid order: FR 1, CDR1, FR2, CDR2, FR3, CDR3, and FR4.

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

“Immune effector cell,” as that term is used herein, refers to a cell that is involved in an immune response, e.g., in the promotion of an immune effector response. Examples of immune effector cells include, but are not limited to, T cells, e.g., alpha/beta T cells and gamma/delta T cells. B cells, natural killer (NK) cells, natural killer T (NK T) cells, and mast cells.

The term “effector function” or “effector response” refers to a specialized function of a cell. Effector function of a T cell, for example, may be cytolytic activity or helper activity including the secretion of cytokines.

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence.” “nucleotide sequence.” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated.” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature. An isolated polynucleotide (ribonucleic acid (RNA), deoxyribonucleic acid (DNA)), or polypeptide is free of the genes/nucleic acids or sequences/amino acids that flank it in its naturally-occurring state.

The compositions and methods of the present invention encompass polypeptides and nucleic acids having the sequences specified, or sequences substantially identical or similar thereto, e.g., sequences at least 80%, 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%, 91%, 92%, 93%, 94%, 95%, 96%, 97%, 98% 99%, 99.5%, 99.9%, or 100% sequence identity to a reference sequence, e.g., a sequence provided herein. In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93%, 94%, 95%, 96%, 97%, 98% 99%, 99.5%, 99.9%, or 100% sequence identity to a reference sequence, e.g., a sequence provided herein.

The term “variant” refers to a polypeptide that has a substantially identical amino acid sequence to a reference amino acid sequence, or is encoded by a substantially identical nucleotide sequence. In some embodiments, the variant is a functional variant. In some embodiments, a TCRβV variant can bind to TCRα and form a TCR α:β complex.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

Calculations of homology or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4. The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As used herein, the term “molecule” as used in, e.g., antibody molecule, cytokine molecule, receptor molecule, includes full-length, naturally-occurring molecules, as well as variants, e.g., functional variants (e.g., truncations, fragments, mutated (e.g., substantially similar sequences) or derivatized form thereof), so long as at least one function and/or activity of the unmodified (e.g., naturally-occurring) molecule remains.

As used herein, the term “mutation” refers to an alteration in the nucleotide sequence of the genome of an organism, virus, or extrachromosomal DNA. In some embodiments, the mutation may be a large-scale mutation, such as amplifications (or gene duplications) or repetitions of a chromosomal segment, deletions of large chromosomal regions, chromosomal rearrangements (e.g., chromosomal translocations, chromosomal inversions, non-homologous chromosomal crossover, and interstitial deletions), and loss of heterozygosity. In some embodiments, the mutation may be a small-scale mutation, such as insertions, deletions, and substitution mutations. As used herein, the term “substitution mutation” refers to the transition that exchange a single nucleotide for another.

“Interleukin-2” also known as IL2, IL-2, IL 2, TCGF, lymphokine, and interleukin 2, as referred to herein, includes any of the recombinant or naturally-occurring forms of IL-2 or variants or homologs thereof that have or maintain IL-2 activity (e.g., at least 40% 50%, 60%, 70%, 80%, 90%, 95%, 96%, 97%, 98%, 99% or 100% activity). In some aspects, the variants or homologs have at least 40%, 45%, 50%, 55%, 60%, 65%, 70%, 75%, 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring IL-2. In some embodiments, IL-2 is substantially identical to the protein identified by the UniProt reference number P60568 or a variant or homolog having substantial identity thereto.

### Anti-TCRβV Antibodies

### Human T Cell Receptor (TCR) Complex

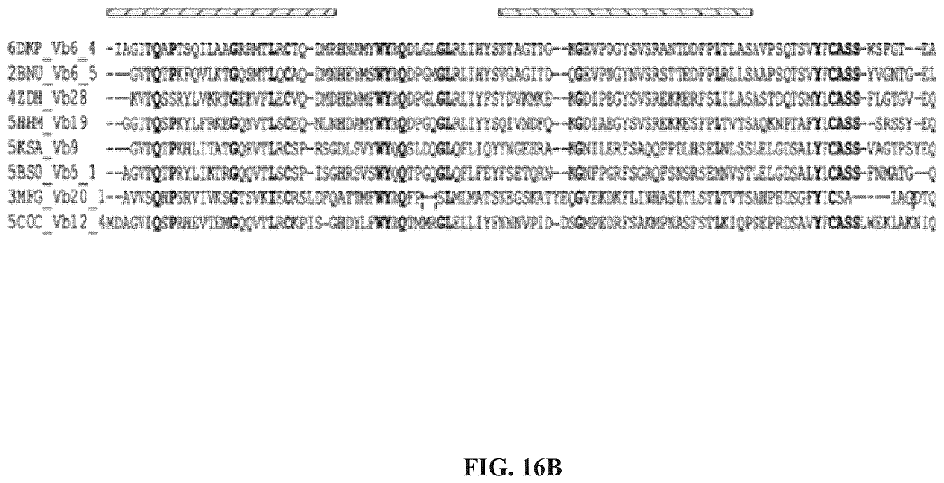

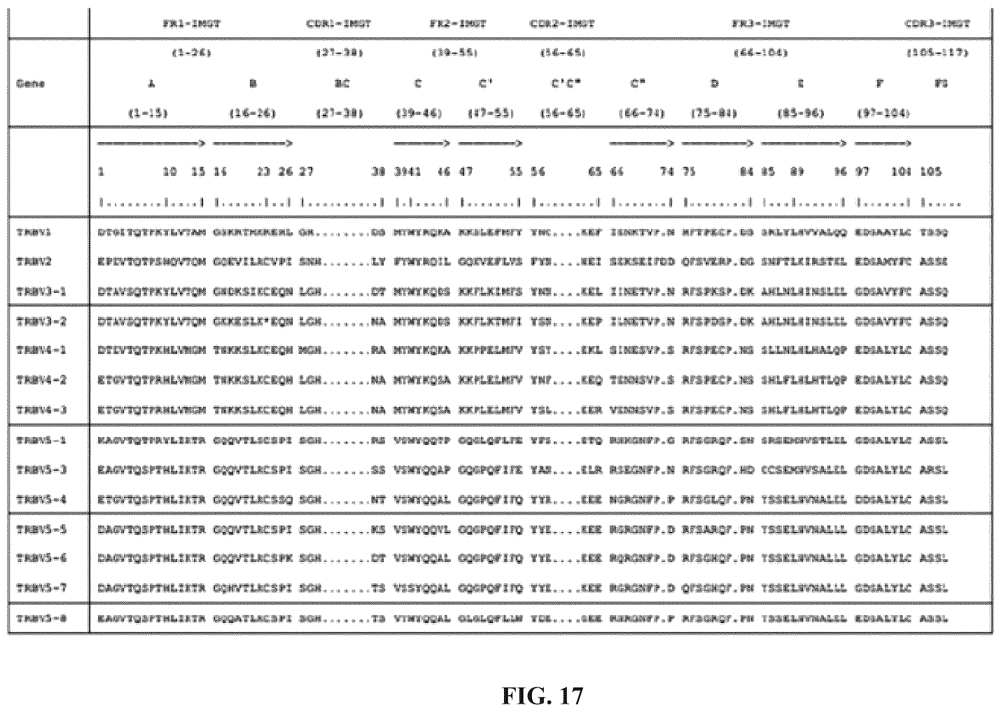

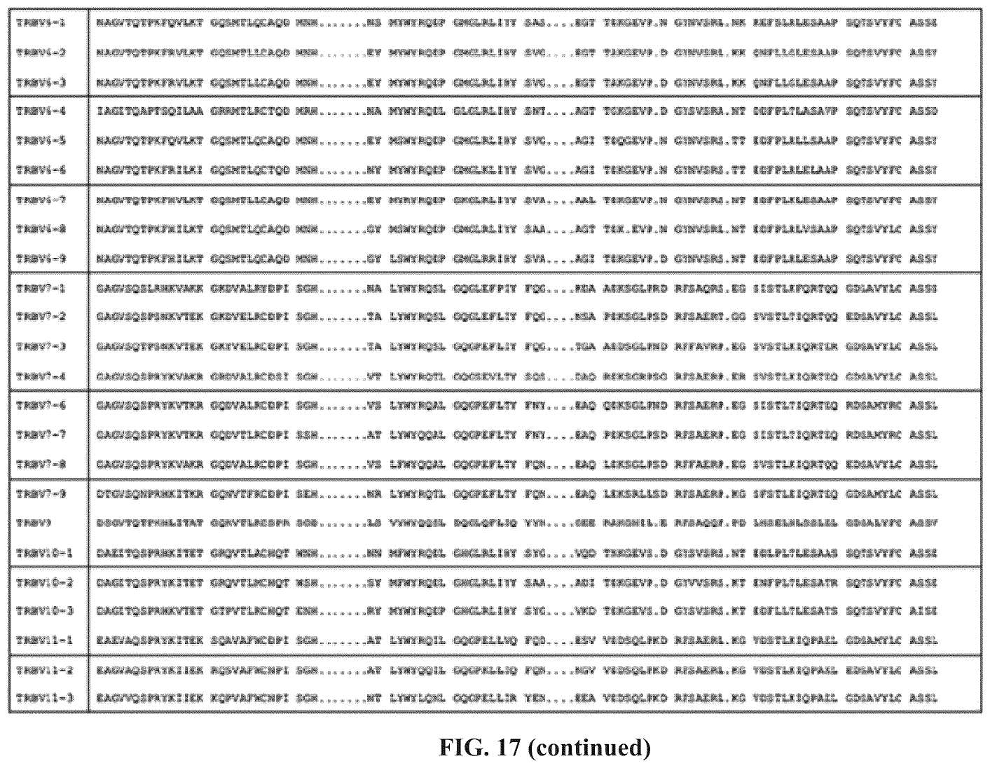

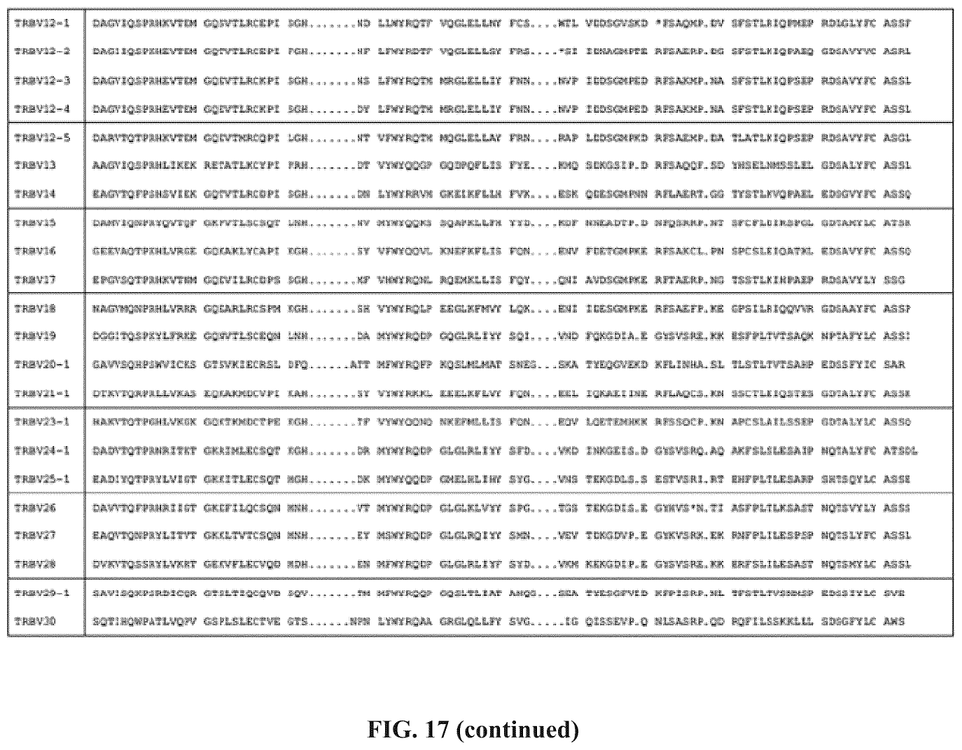

TCR is a disulfide-linked membrane-anchored heterodimeric protein normally consisting of the highly variable alpha (α) and beta (β) chains expressed as part of a complex with the invariant CD3 chain molecules, TCR on αβ T cells is formed by a heterodimer of one alpha chain and one beta chain. Each alpha or beta chain consists of a constant domain and a highly variable domain classified as the Immunoglobulin superfamily (IgSF) fold. The TCRβV chains can be further classified into 30 subfamilies (TCRvβ1-30). Despite their high structural and functional homology, the amino acid sequence homology in the TCRvβ genes is very low. Only 4 amino acids out of approximately 95 are identical while 10 additional amino acids are conserved among all subfamilies (see, an alignment of TCRvβ amino acid sequences in FIG. 17). Nevertheless, TCRβ formed between alpha and beta chains of highly diverse sequences show a remarkable structural homology (FIGS. 16A and 16B) and elicit a similar function, e.g., activation of T cells.

T cell receptors (TCR) can be found on the surface of T cells. TCRβ recognize antigens, e.g., peptides, presented on, e.g., bound to, major histocompatibility complex (MHC) molecules on the surface of cells, e.g., antigen-presenting cells. TCRβ are heterodimeric molecules and can comprise an alpha chain, a beta chain, a gamma chain or a delta chain. TCRβ comprising an alpha chain and a beta chain are also referred to as TCRαβ. The TCR beta chain consists of the following regions (also known as segments): variable (V), diversity (D), joining (J) and constant (C) (see Mayer G. and Nyland J. (2010) Chapter 10: Major Histocompatibility Complex and T-cell Receptors-Role in Immune Responses. In: Microbiology and Immunology on-line, University of South Carolina School of Medicine). The TCR alpha chain consists of V. J and C regions. The rearrangement of the T-cell receptor (TCR) through somatic recombination of V (variable). D (diversity), J (joining), and C (constant) regions is a defining event in the development and maturation of a T cell, TCR gene rearrangement takes place in the thymus.

TCRβ can comprise a receptor complex, known as the TCR complex, which comprises a TCR heterodimer comprising of an alpha chain and a beta chain, and dimeric signaling molecules, e.g., CD3 co-receptors, e.g., CD3δ/ε, and/or CD3γ/ε.

As used herein, the term “T cell receptor beta variable chain” or “TCRβV,” refers to an extracellular region of the T cell receptor beta chain which comprises the antigen recognition domain of the T cell receptor. The term TCRβV includes isoforms, mammalian, e.g., human TCRβV, species homologs of human and analogs comprising at least one common epitope with TCRβV. Human TCRβV comprises a gene family comprising subfamilies including, but not limited to: a TCRβ V6 subfamily, a TCRβ V10 subfamily, a TCRβ V12 subfamily, a TCRβ V5 subfamily, a TCRβ V7 subfamily, a TCRβV11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, a TCRβ V29 subfamily, a TCRβ V1 subfamily, a TCRβ V17 subfamily, a TCRβ V21 subfamily, a TCRβ V23 subfamily, or a TCRβ V26 subfamily, as well as family members of said subfamilies, and variants thereof (e.g., a structural or functional variant thereof). In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, TCRβV comprises TCRβ V6-5*01, or a variant thereof, e.g., a variant having 85%, 90%, 95%, 99% or more identity the naturally-occurring sequence, TCRβ V6-5*01 is also known as TCRvβ65; TCRvβ6S5: TCRvβ13S1, or TCRβ V13.1. The amino acid sequence of TCRβ V6-5*01, e.g., human TCRβ V6-5*01, is known in that art, e.g., as provided by IMGT ID L36092. In some embodiments, TCRβ V6-5*01 is encoded by the nucleic acid sequence of SEQ ID NO: 43, or a sequence having 85%, 90%, 95%, 99% or more identity thereof. In some embodiments, TCRβ V6-5*01 comprises the amino acid sequence of SEQ ID NO: 44, or a sequence having 85%, 90%, 95%, 99% or more identity thereof.

SEQ ID NO: 43
ATGAGCATCGGCCTCCTGTGCTGTGCAGCCTTGTCTCTCCTGTGGGCAGGTCCAGTGAATGCT
GGTGTCACTCAGACCCCAAAATTCCAGGTCCTGAAGACAGGACAGAGCATGACACTGCAGT
GTGCCCAGGATATGAACCATGAATACATGTCCTGGTATCGACAAGACCCAGGCATGGGGCTG
AGGCTGATTCATTACTCAGTTGGTGCTGGTATCACTGACCAAGGAGAAGTCCCCAATGGCTA
CAATGTCTCCAGATCAACCACAGAGGATTTCCCGCTCAGGCTGCTGTCGGCTGCTCCCTOCCA
GACATCTGTGTACTTCTGTGCCAGCAGTTACTC
SEQ ID NO: 44
MSIGLLCCAALSLLWAGPVNAGVTQTPKFQVLKTGQSMTLQCAQDMNHEYMSWYRQDPGMG
LRLIHY-SVGAGITDQGEVPNGYNVSRSTTEDFPLRLLSAAPSQTSVYFCASSY

### TCR Beta V (TCRβV)

Diversity in the immune system enables protection against a huge array of pathogens. Since the germline genome is limited in size, diversity is achieved not only by the process of V(D)J recombination but also by junctional (junctions between V-D and D-J segments) deletion of nucleotides and addition of pseudo-random, non-templated nucleotides. The TCR beta gene undergoes gene arrangement to generate diversity.

The TCR V beta repertoire varies between individuals and populations because of, e.g., 7 frequently occurring inactivating polymorphisms in functional gene segments and a large insertion/deletion-related polymorphism encompassing 2 V beta gene segments.

Provided herein are, inter alia, antibody molecules and fragments thereof, that bind, e.g., specifically bind, to a human TCR beta V chain (TCRβV), e.g., a TCRβV gene family (also referred to as a group), e.g., a TCRβV subfamily (also referred to as a subgroup), e.g., as described herein. TCR beta V families and subfamilies are known in the art, e.g., as described in Yassai et al., (2009) Immunogenetics 61(7) pp: 493-502; Wei S. and Concannon P. (1994) Human Immunology 41(3) pp: 201-206. The antibodies described herein can be recombinant antibodies, e.g., recombinant non-murine antibodies, e.g., recombinant human or humanized antibodies.

The terms TCRBV, TCRVB, TRBV, TCRβV, TCRVβ, or TRβV are used interchangeably herein and refer to a TCR beta V chain, e.g., as described herein.

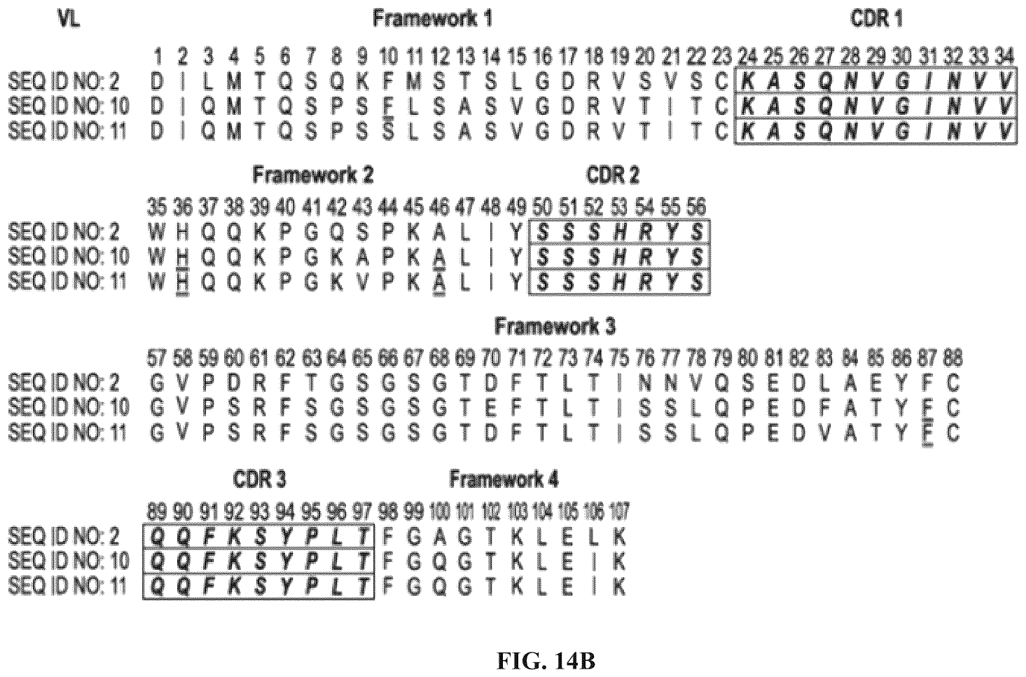

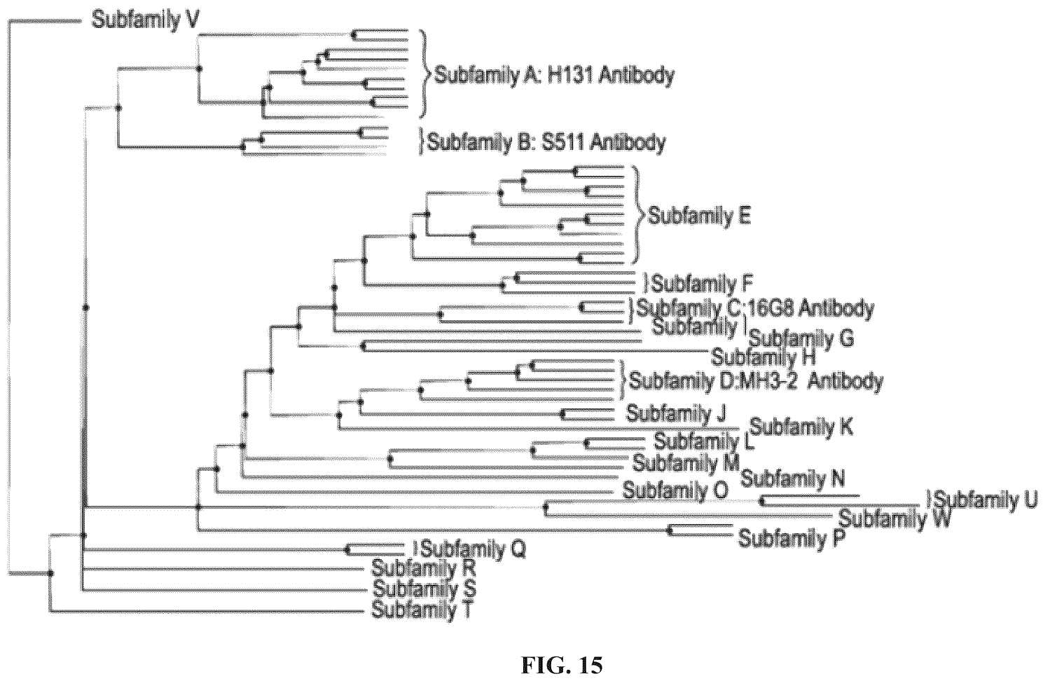

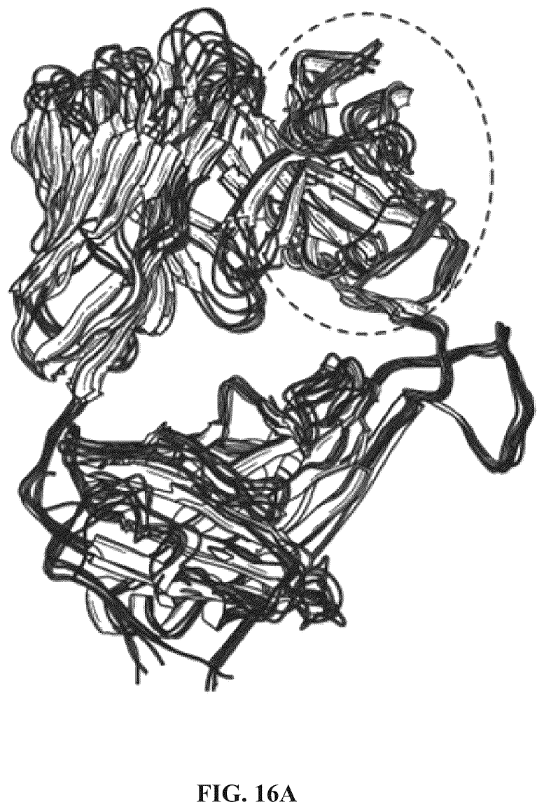

In some embodiments, provided herein is an anti-TCRβV antibody molecule that binds to human TCRβV, e.g., a TCRβV family, e.g., gene family or a variant thereof. In some embodiments a TCRBV gene family comprises one or more subfamilies, e.g., as described herein, e.g., in FIG. 14. Table 8A or Table 8B. In some embodiments, the TCRβV gene family comprises: a TCRβ V6 subfamily, a TCRβ V10 subfamily, a TCRβ V12 subfamily, a TCRβ V5 subfamily, a TCRβ V7 subfamily, a TCRβ V 11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, a TCRβ V29 subfamily, a TCRβ V1 subfamily, a TCRβ V17 subfamily, a TCRβ V21 subfamily, a TCRβ V23 subfamily, or a TCRβ V26 subfamily.

In some embodiments, TCRβ subfamily is also known as TCRβ V13.1. In some embodiments, the TCRβ V6 subfamily comprises: TCRβ V6-4*01, TCRβ V6-4*02, TCRβ V6-9*01, TCRβ V6-8*01. TCRβ V6-5*01, TCRβ V6-6*02, TCRβ V6-6*01, TCRβ V6-2*01, TCRβ V6-3*01 or TCRβ V6-1*01, or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments. TCRβ V6 comprises TCRβ V6-9*01, or a variant thereof. In some embodiments, TCRβ V6 comprises TCRβ V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments, TCRβ V6 comprises TCRβ V6-6*01, or a variant thereof. In some embodiments, TCRβ V6 comprises TCRβ V6-2*01, or a variant thereof. In some embodiments, TCRβ V6 comprises TCRβ V6-3*01, or a variant thereof. In some embodiments, TCRβ V6 comprises TCRβ V6-1*01, or a variant thereof.

In some embodiments, TCRβ V6 comprises TCRβ V6-5*01, or a variant thereof. In some embodiments, TCRβ V6, e.g., TCRβ V6-5*01, is recognized, e.g., bound, by SEQ ID NO: 1 and/or SEQ ID NO: 2. In some embodiments, TCRβ V6, e.g., TCRβ V6-5*01, is recognized, e.g., bound, by SEQ ID NO: 9 and/or SEQ ID NO: 10. In some embodiments, TCRβ V6 is recognized, e.g., bound, by SEQ ID NO: 9 and/or SEQ ID NO: 11.

In some embodiments, TCRβ V10 subfamily is also known as TCRβ V12. In some embodiments, the TCRβ V10 subfamily comprises: TCRβ V10-1*01, TCRβ V10-1*02, TCRβ V10-3*01 or TCRβ V10-2*01, or a variant thereof.

In some embodiments, TCRβ V12 subfamily is also known as TCRβ V8.1. In some embodiments, the TCRβ V12 subfamily comprises: TCRβ V12-4*01, TCRβ V12-3*01, or TCRβ V12-5*01, or a variant thereof. In some embodiments, TCRβ V12 is recognized, e.g., bound, by SEQ ID NO: 15 and/or SEQ ID NO: 16. In some embodiments, TCRβ V12 is recognized, e.g., bound, by any one of SEQ ID NOs 23-25, and/or any one of SEQ ID NO: 26-30.

In some embodiments, the TCRβ V5 subfamily is chosen from: TCRβ V5-5*01, TCRβ V5-6*01. TCRβ V5-4*01, TCRβ V5-8*01, TCRβ V5-1*01, or a variant thereof.

In some embodiments, the TCRβ V7 subfamily comprises TCRβ V7-7*01, TCRβ V7-6*01. TCRβ V7-8*02, TCRβ V7-4*01, TCRβ V7-2*02, TCRβ V7-2*03, TCRβ V7-2*01, TCRβ V7-3*01. TCRβ V7-9*03, or TCRβ V7-9*01, or a variant thereof.

In some embodiments, the TCRβ V11 subfamily comprises: TCRβ V11-1*01, TCRβ V11-2*01 or TCRβ V11-3*01, or a variant thereof. In some embodiments, the TCRβ V14 subfamily comprises TCRβ V14*01, or a variant thereof. In some embodiments, the TCRβ V16 subfamily comprises TCRβ V16*01, or a variant thereof. In some embodiments, the TCRβ V18 subfamily comprises TCRβ V18*01, or a variant thereof. In some embodiments, the TCRβ V9 subfamily comprises TCRβ V9*01 or TCRβ V9*02, or a variant thereof. In some embodiments, the TCRβ V13 subfamily comprises TCRβ V13*01, or a variant thereof. In some embodiments, the TCRβ V4 subfamily comprises TCRβ V4-2*01, TCRβ V4-3*01, or TCRβ V4-1*01, or a variant thereof. In some embodiments, the TCRβ V3 subfamily comprises TCRβ V3-1*01, or a variant thereof. In some embodiments, the TCRβ V2 subfamily comprises TCRβ V2*01, or a variant thereof. In some embodiments, the TCRβ V15 subfamily comprises TCRβ V15*01, or a variant thereof. In some embodiments, the TCRβ V30 subfamily comprises TCRβ V30*01, or TCRβ V30*02, or a variant thereof. In some embodiments, the TCRβ V19 subfamily comprises TCRβ V19*01, or TCRβ V19*02, or a variant thereof. In some embodiments, the TCRβ V27 subfamily comprises TCRβ V27*01, or a variant thereof. In some embodiments, the TCRβ V28 subfamily comprises TCRβ V28*01, or a variant thereof. In some embodiments, the TCRβ V24 subfamily comprises TCRβ V24-1*01, or a variant thereof. In some embodiments, the TCRβ V20 subfamily comprises TCRβ V20-1*01, or TCRβ V20-1*02, or a variant thereof. In some embodiments, the TCRβ V25 subfamily comprises TCRβ V25-1*01, or a variant thereof. In some embodiments, the TCRβ V29 subfamily comprises TCRβ V29-1*01, or a variant thereof.

Exemplary amino acid sequences for TCRβV subfamily members can be found on the ImMunoGeneTics Information System website: http://www.imgt.org/, or in a similar resource.

### Anti-TCRβV Antibodies

Current anti-TCRβV antibodies designed to redirect T cells to promote tumor cell lysis for cancer immunotherapy typically utilize antibody fragments (Fab, scFv, VH, single domain antibody, etc.) that are derived from monoclonal antibodies (mAb) directed against the CD3e subunit of the T cell receptor (TCR). However, there are limitations to this approach which may prevent the full realization of the therapeutic potential for such bispecific constructs. Previous studies have shown that even low “activating” doses of anti-CD3e mAb can cause long-term T cell dysfunction and exert immunosuppressive effects. In addition, anti-CD3e mAbs have been associated with side effects that result from massive T cell activation. The large number of activated T cells secrete substantial amounts of cytokines, the most important of which is Interferon gamma (IFNγ). This excess amount of IFNγ in turn activates macrophages which then overproduce proinflammatory cytokines such as IL-1beta, IL-6, IL-10 and TNF-alpha, causing a “cytokine storm” known as the cytokine release syndrome (CRS) (Shimabukuro-Vornhagen et al., J Immunother Cancer. 2018 Jun. 15; 6(1): 56, herein incorporated by reference in its entirety). Thus, the need exists for developing antibodies that are capable of binding and activating only a subset of effector T cells, e.g., to re-duce the CRS and/or neurotoxicity (NT).

Described herein are molecules targeting the TCRβV chain of TCR and methods thereof. Without wishing to be bound by theory, such molecules are capable of binding, activating, and/or expanding only a subset of T cells, avoiding or reducing CRS and/or NT and minimizing potential immunosuppressive effects of anti-CD3 mAbs.

Described herein is a class of antibodies, i.e., anti-TCRβV antibody molecules as described herein, which despite having low sequence similarity (e.g., low sequence identity among the different antibody molecules that recognize different TCRβV subfamilies), recognize a structurally conserved, yet sequence-wise variable, region, e.g., domain, on the TCRβV protein (as denoted by the circled area in FIG. 16A) and have a similar function (e.g., activation of T cells and a similar cytokine profile as described herein). Thus, the anti-TCRβV antibody molecules as described herein share a structure-function relationship.

Without wishing to be bound by theory, in some embodiments, the anti-TCRβV antibody molecules as described herein bind to an outward facing epitope of a TCRβV protein when it is in a complex with a TCRalpha protein, e.g., as denoted by the circled area in FIG. 16A. In some embodiments, the anti-TCRβV antibody molecules as described herein recognize (e.g., bind to), a domain (e.g., an epitope) on the TCRβV protein that is: (1) structurally conserved among different TCRβV subfamilies; and (2) has minimal sequence identity among the different TCRβV subfamilies. As shown in FIG. 17. TCRβV proteins from the different TCRβV subfamilies share minimal sequence similarity. However, as shown in FIG. 16A-16B, TCRβV proteins which have minimal sequence similarity, share a similar 3D conformation and structure.

The alignment of TCRβV amino acid sequences in FIG. 17 underscores the diversity of TCR sequences. In particular, the TCRvβ sequences from different subfamilies are considerably different from each other.

Various anti-TCRβV targeting different subfamilies of TCRβV may bind to different structural regions on the TCRβV, or they may bind to similar structural regions on the TCRβV. In some embodiments, the anti-TCRβV antibody bind to a germline encoded region of the TCRβV. In some embodiments, the anti-TCRβV antibody bind to a hypervariable region 4 (HV4) of the TCRβV. In some embodiments, the anti-TCRβV antibody bind to a complementarity-determining region 2 (CDR2) of the TCRβV. In some embodiments, the anti-TCRβ antibody bind to the regions directly after the HV4 region. In some embodiments, the anti-TCRβV antibody bind to the region between the CDR2 and the HV4 regions.

In some embodiments, the anti-TCRβV antibody molecules as described herein do not recognize, e.g., bind to, an interface of a TCRβV:TCRalpha complex. In some embodiments, the anti-TCRβV antibody molecules as described herein do not recognize, e.g., bind to, a constant region of a TCRβV protein. An exemplary antibody that binds to a constant region of a TCRβV region is JOVI.1 as described in Vincy et al., (Hybridoma. 1992 December; 11(6):701-13). In some embodiments, the anti-TCRβV antibody molecules as described herein do not recognize, e.g., bind to one or more (e.g., all) of a complementarity determining region (e.g., CDR1, CDR2 and/or CDR3) of a TCRβV protein.

Provided herein are, inter aha, antibody molecules directed to the variable chain of the beta subunit of TCR (TCRβV) which bind and, e.g., activate a subset of T cells. The anti-TCRβV antibody molecules as described herein result in lesser or no production of cytokines associated with CRS, e.g., IL-6, IL-1beta, IL-10 and TNF alpha; and enhanced and/or delayed production of IL-2 and IFNγ. In some embodiments, the anti-TCRβV antibodies as described herein have a cytokine profile, e.g., as described herein, which differs from a cytokine profile of a T cell engager that binds to a receptor or molecule other than a TCRβV region (“a non-TCRβV-binding T cell engager”). In some embodiments, the non-TCRβV-binding T cell engager comprises an antibody that binds to a CD3 molecule (e.g., CD3 epsilon (CD3e) molecule); or a TCR alpha (TCRα) molecule. In some embodiments, the non-TCRβV-binding T cell engager is an OKT3 antibody or an SP34-2 antibody.

In some embodiments, the anti-TCRβV antibodies as described herein result in expansion of TCRβV+ T cells, e.g., a subset of memory effector T cells known as TEMRA. Without wishing to be bound by theory, it is believed that in some embodiments. TEMRA cells can promote tumor cell lysis but not CRS. Accordingly, provided herein are methods of making said anti-TCRβV antibody molecules and uses thereof. Also described herein are multispecific molecules, e.g., bispecific molecules comprising said anti-TCRβV antibody molecules. In some embodiments, compositions comprising anti-TCRβV antibody molecules of the present disclosure, can be used, e.g., to: (1) activate and redirect T cells to promote tumor cell lysis for cancer immuno-therapy; and/or (2) expand TCRβV+ T cells. In some embodiments, compositions comprising anti-TCRβV antibody molecules as described herein limit the harmful side-effects of CRS and/or NT, e.g., CRS and/or NT associated with anti-CD3e targeting.

In some embodiments, the anti-TCRβV antibody molecule binds to one or more of TCRvβ2, TCRvβ3-1, TCRvβ4-1, TCRvβ4-2, TCRvβ4-3, TCRvβ5-1, TCRvβ5-4, TCRvβ5-5, TCRv5-6, TCRvβ5-8, TCRvβ6-1, TCRvβ6-2, TCRvβ6-3, TCRvβ6-4, TCRvβ6-5, TCRvβ6-6, TCRvβ6-8, TCRvβ6-9, TCRvβ7-2, TCRvβ7-3, TCRvβ7-4, TCRvβ7-6, TCRvβ7-7, TCRvβ37-8, TCRvβ37-9, TCRvβ9, TCRvβ10-1, TCRvβ10-2, TCRvβ10-3, TCRvβ11-1, TCRvβ11-2, TCRvβ11-3, TCRvβ12-3, TCRvβ12-4, TCRvβ12-5, TCRvβ13, TCRvβ4, TCRvβ15, TCRvβ6, TCRvβ8, TCRvβ9, TCRv20-1, TCRvβ24-1, TCRv25-1, TCRvβ27, TCRvβ28, TCRvβ29-1 and TCRvβ30. In some embodiments, the anti-TCRβV antibody molecule binds to one or more of TCRvβ6-1, TCRvβ6-2, TCRvβ6-3, TCRvβ6-4, TCRvβ6-5, TCRvβ6-6. TCRvβ6-8 and TCRvβ6-9. In some embodiments, the anti-TCRβV antibody molecule is an anti-TCRvβ32, anti-TCRvβ3-1, anti-TCRvβ4-1, anti-TCRvβ4-2, anti-TCRvβ4-3, anti-TCRvβ5-1, anti-TCRvβ5-4, anti-TCRvβ5-5, anti-TCRvβ5-6, anti-TCRvβ5-8, anti-TCRvβ6-1, anti-TCRvβ6-2, anti-TCRvβ6-3, anti-TCRvβ64, anti-TCRvβ6-5, anti-TCRvβ6-6, anti-TCRvβ6-8, anti-TCRvβ6-9, anti-TCRvβ7-2, anti-TCRvβ7-3, anti-TCRvβ7-4, anti-TCRvβ7-6, anti-TCRvβ7-7, anti-TCRv37-8, anti-TCRvβ7-9, anti-TCRvβ39, anti-TCRvβ10-1, anti-TCRv10-2, anti-TCRvβ10-3, anti-TCRvβ11-1, anti-TCRvβ11-2, anti-TCRvβ11-3, anti-TCRvβ12-3, anti-TCRvβ12-4, anti-TCRvβ12-5, anti-TCRvβ13, anti-TCRvβ14, anti-TCRvβ15, anti-TCRvβ16, anti-TCRvβ18, anti-TCRv19, anti-TCRvβ320-1, anti-TCRvβ24-1, anti-TCRvβ25-1, anti-TCRvβ27, anti-TCRvβ28, anti-TCRvβ29-1, or anti-TCRvβ30. Exemplary anti-TCRβV antibody molecules and the corresponding TCRβV subfamilies recognized by said anti-TCRβV antibody molecules are disclosed in Table 10A.

In some embodiments, the anti-TCRβV antibody molecule binds specifically to TCRvβ2. TCRvβ3-1, TCRvβ34-1, TCRvβ4-2, TCRvβ4-3, TCRvβ5-1, TCRvβ5-4, TCRvβ5-5, TCRvβ05-6, TCRvβ5-8, TCRvβ6-1, TCRvβ6-2, TCRvβ6-3, TCRvβ6-4, TCRvβ6-5, TCRvβ6-6, TCRvβ6-8, TCRvβ6-9. TCRvβ7-2, TCRvβ7-3, TCRvβ7-4, TCRvβ7-6, TCRv7-7, TCRvβ07-8, TCRvβ7-9, TCRvβ9, TCRvβ10-1. TCRvβ10-2, TCRvβ10-3, TCRvβ11-1, TCRvβ1-2, TCRvβ11-3, TCRvβ12-3, TCRvβ12-4, TCRvβ12-5, TCRvβ13, TCRvβ14, TCRvβ15, TCRvβ16, TCRvβ18, TCRvβ19, TCRvβ20-1, TCRvβ24-1, TCRvβ25-1, TCRvβ27, TCRvβ28, TCRvβ29-1 or TCRvβ30. In some embodiments, the anti-TCRβV antibody molecule binds specifically to TCRvβ6-1. In some embodiments, the anti-TCRβV antibody molecule binds specifically to TCRv6-2. In some embodiments, the anti-TCRβV antibody molecule binds specifically to TCRvβ6-3. In some embodiments, the anti-TCRβV antibody molecule binds specifically to TCRvβ6-4. In some embodiments, the anti-TCRβV antibody molecule binds specifically to TCRvβ6-5. In some embodiments, the anti-TCRβV antibody molecule binds specifically to TCRvβ6-6. In some embodiments, the anti-TCRβV antibody molecule binds specifically to TCRvβ6-8. In some embodiments, the anti-TCRβV antibody molecule binds specifically to TCRvβ6-9.

In some embodiments, the anti-TCRβV antibody molecule does not bind to TCRβ V12, or binds to TCRβ V12 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the 16G8 murine antibody or a humanized version thereof as de-scribed in U.S. Pat. No. 5,861,155.

In some embodiments, the anti-TCRβV antibody molecule binds to TCRβ V12 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155.

In some embodiments, the anti-TCRβV antibody molecule binds to a TCRβV region other than TCRβ V12 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155.

In some embodiments, the anti-TCRβV antibody molecule does not comprise the CDRs of the Antibody B murine antibody.

In some embodiments, the anti-TCRβV antibody molecule does not bind to TCRβ V5-5*01 or TCRβ V5-1*01, or binds to TCRβ V5-5*01 or TCRβ V5-1*01 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155.

In some embodiments, the anti-TCRβV antibody molecule binds to TCRβ V5-5*01 or TCRβ V5-1*01 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as de-scribed in U.S. Pat. No. 5,861,155.

In some embodiments, the anti-TCRβV antibody molecule binds to a TCRβV region other than TCRβ V5-5*01 or TCRβ V5-1*01 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80, 90% or about 2-, 5-, or 10-fold) the affinity and/or binding specificity of the TM23 murine antibody or a humanized version thereof as described in U.S. Pat. No. 5,861,155.

In some embodiments, the anti-TCRβV antibody molecule does not comprise the CDRs of the TM23 murine antibody.

In some embodiments, the light or the heavy chain variable framework (e.g., the region encompassing at least FR1, FR2, FR3, and optionally FR4) of the anti-TCRβV antibody molecule, e.g., anti-TCRβ V6 (e.g., anti-TCRβ V6-5*01) antibody molecule can be chosen from: (a) a light or heavy chain variable framework including at least 80%, 85%, 87% 90%, 92%, 93%, 95%, 97%, 98%, or 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In some embodiments, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

In some embodiments, the anti-TCRβV antibody molecule, e.g., anti-TCRβ V6 (e.g., anti-TCRβ V6-5*01) antibody molecule, comprises a heavy chain variable domain having at least one, two, three, four, five, six, seven, ten, fifteen, twenty or more changes, e.g., amino acid substitutions or deletions, from an amino acid sequence of any one of A-H.1 to A-H.85, e.g., A-H.1, A-H.2 or A-H.68, e.g., the amino acid sequence of the FR region in the entire variable region, e.g., shown in FIG. 14A, or in SEQ ID NO: 9.

Alternatively, or in combination with the heavy chain substitutions described herein, the anti-TCRβV antibody molecule, e.g., anti-TCRβ V6 (e.g., anti-TCRβ V6-5*01) antibody molecule, comprises a light chain variable domain having at least one, two, three, four, five, six, seven, ten, fifteen, twenty or more amino acid changes, e.g., amino acid substitutions or deletions, from an amino acid sequence of any one of A-H.1 to A-H.85, e.g., A-H.1, A-H.2 or A-H.68, e.g., the amino acid sequence of the FR region in the entire variable region, e.g., shown in FIG. 14B, or in SEQ ID NO: 10 or SEQ ID NO: 11.

In some embodiments, the anti-TCRβV antibody molecule, e.g., anti-TCRβ V6 (e.g., anti-TCRβ V6-5*01) antibody molecule, includes one, two, three, or four heavy chain framework regions shown in FIG. 14A, or a sequence substantially identical thereto.

In some embodiments, the anti-TCRβV antibody molecule, e.g., anti-TCRβ V6 (e.g., anti-TCRβ V6-5*01) antibody molecule, includes one, two, three, or four light chain framework regions shown in FIG. 14B, or a sequence substantially identical thereto.

In some embodiments, the anti-TCRβV antibody molecule, e.g., anti-TCRβ V6 (e.g., anti-TCRβ V6-5*01) antibody molecule, comprises the light chain framework region 1 of A-H.1 or A-H.2, e.g., as shown in FIG. 14B.

In some embodiments, the anti-TCRβV antibody molecule, e.g., anti-TCRβ V6 (e.g., anti-TCRβ V6-5*01) antibody molecule, comprises the light chain framework region 2 of A-H.1 or A-H.2, e.g., as shown in FIG. 14B.

In some embodiments, the anti-TCRβV antibody molecule, e.g., anti-TCRβ V6 (e.g., anti-TCRβ V6-5*0l) antibody molecule, comprises the light chain framework region 3 of A-H.1 or A-H.2, e.g., as shown in FIG. 14B.

In some embodiments, the anti-TCRβV antibody molecule, e.g., anti-TCRβ V6 (e.g., anti-TCRβ V6-5*01) antibody molecule, comprises the light chain framework region 4 of A-H.1 or A-H.2, e.g., as shown in FIG. 14B.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 1 (FR1), comprising a change, e.g., a substitution (e.g., a conservative substitution) at position 10 according to Kabat numbering. In some embodiments, the FR1 comprises a Phenylalanine at position 10, e.g., a Serine to Phenyalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 2 (FR2), comprising a change, e.g., a substitution (e.g., a conservative substitution) at a position as described herein according to Kabat numbering. In some embodiments, FR2 comprises a Histidine at position 36, e.g., a substitution at position 36 according to Kabat numbering, e.g., a Tyrosine to Histidine substitution. In some embodiments. FR2 comprises an Alanine at position 46, e.g., a substitution at position 46 according to Kabat numbering, e.g., an Arginine to Alanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising a framework region. e.g., framework region 3 (FR3), comprising a change, e.g., a substitution (e.g., a conservative substitution) at a position as described herein according to Kabat numbering. In some embodiments, FR3 comprises a Phenyalanine at position 87, e.g., a substitution at position 87 according to Kabat numbering, e.g., a Tyrosine to Phenyalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*0l) antibody molecule, comprises a light chain variable domain comprising: (a) a framework region 1 (FR1) comprising a Phenylalanine at position 10, e.g., a substitution at position 10 according to Kabat numbering, e.g., a Serine to Phenvalanine substitution; (b) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (c) a framework region 3 (FR3) comprising a Phenylalanine at position 87, e.g., a substitution at position 87 according to Kabat numbering, e.g., a Tyrosine to Phenyalanine substitution, e.g., as shown in the amino acid sequence of SEQ ID NO: 10. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (b) a framework region 3 (FR3) comprising a Phenylalanine at position 87, e.g., a substitution at position 87 according to Kabat numbering, e.g., a Tyrosine to Phenyalanine substitution, e.g., as shown in the amino acid sequence of SEQ ID NO: 11. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 1 (FR1) comprising a change, e.g., a substitution (e.g., a conservative substitution) at one or more (e.g., all) positions as described herein according to Kabat numbering; (b) a framework region 2 (FR2) comprising a change, e.g., a substitution (e.g., a conservative substitution) at one or more (e.g., all) position as described herein according to Kabat numbering and (c) a framework region 3 (FR3) comprising a change, e.g., a substitution (e.g., a conservative substitution) at one or more (e.g., all) position as described herein according to Kabat numbering. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises the heavy chain framework region 1 of A-H.1 or A-H.2, e.g., as shown in FIG. 14A. In some embodiments, the anti-TCRβV antibody molecule, e.g., anti-TCRβ V6 (e.g., anti-TCRβ V6-5*01) antibody molecule, comprises the heavy chain framework region 2 of A-H.1 or A-H.2, e.g., as shown in FIG. 14A. In some embodiments, the anti-TCRβV antibody molecule, e.g., anti-TCRβ V6 (e.g., anti-TCRβ V6-5*01) antibody molecule, comprises the heavy chain framework region 3 of A-H.1 or A-H.2, e.g., as shown in FIG. 14A. In some embodiments, the anti-TCRβV antibody molecule, e.g., anti-TCRβ V6 (e.g., anti-TCRβ V6-5*01) antibody molecule, comprises the heavy chain framework region 4 of A-H.1 or A-H.2, e.g., as shown in FIG. 14A.

In some embodiments, the anti-TCRβV antibody molecule, e.g., anti-TCRβ V6 (e.g., anti-TCRβ V6-5*01) antibody molecule, comprises a heavy chain variable domain comprising a framework region. e.g., framework region 3 (FR3), comprising a change, e.g., a substitution (e.g., a conservative substitution) at a position as described herein according to Kabat numbering. In some embodiments. FR3 comprises a Threonine at position 73, e.g., a substitution at position 73 according to Kabat numbering, e.g., a Glutamic Acid to Threonine substitution. In some embodiments. FR3 comprises a Glycine at position 94, e.g., a substitution at position 94 according to Kabat numbering, e.g., an Arginine to Glycine substitution. In some embodiments, the substitution is relative to a human germline heavy chain framework region sequence.

In some embodiments, the anti-TCRβV antibody molecule, e.g., anti-TCRβ V6 (e.g., anti-TCRβ V6-5*01) antibody molecule, comprises a heavy chain variable domain comprising a framework region 3 (FR3) comprising a Threonine at position 73, e.g., a substitution at position 73 according to Kabat numbering, e.g., a Glutamic Acid to Threonine substitution, and a Glycine at position 94, e.g., a substitution at position 94 according to Kabat numbering, e.g., a Arginine to Glycine substitution, e.g., as shown in the amino acid sequence of SEQ ID NO: 10.

In some embodiments, the anti-TCRβV antibody molecule, e.g., anti-TCRβ V6 (e.g., anti-TCRβ V6-5*01) antibody molecule, comprises the heavy chain framework regions 1-4 of A-H.1 or A-H.2, e.g., SEQ ID NO: 9, or as shown in FIGS. 14A and 14B. In some embodiments, the anti-TCRβV antibody molecule, e.g., anti-TCRβ V6 (e.g., anti-TCRβ V6-5*01) antibody molecule, comprises the light chain framework regions 1-4 of A-H.1, e.g., SEQ ID NO: 10, or as shown in FIGS. 14A and 14B. In some embodiments, the anti-TCRβV antibody molecule, e.g., anti-TCRβ V6 (e.g., anti-TCRβ V6-5*01) antibody molecule, comprises the light chain framework regions 1-4 of A-H.2, e.g., SEQ ID NO: 11, or as shown in FIGS. 14A and 14B. In some embodiments, the anti-TCRβV antibody molecule, e.g., anti-TCRβ V6 (e.g., anti-TCRβ V6-5*0l) antibody molecule, comprises the heavy chain framework regions 1-4 of A-H.1, e.g., SEQ ID NO: 9, and the light chain framework regions 1-4 of A-H.1, e.g., SEQ ID NO: 10, or as shown in FIGS. 14A and 14B. In some embodiments, the anti-TCRβV antibody molecule, e.g., anti-TCRβ V6 (e.g., anti-TCRβ V6-5*0l) antibody molecule, comprises the heavy chain framework regions 1-4 of A-H.2, e.g., SEQ ID NO: 9; and the light chain framework regions 1-4 of A-H.2, e.g., SEQ ID NO: 11, or as shown in FIGS. 14A and 14B.

In some embodiments, the heavy or light chain variable domain, or both, of the anti-TCRβV antibody molecule, e.g., anti-TCRβ V6 (e.g., anti-TCRβ V6-5*01) antibody molecule, includes an amino acid sequence, which is substantially identical to an amino acid as described herein, e.g., at least 80%, 85%, 90%, 92%, 95%, 97%, 98%, 99% or higher identical to a variable region of an antibody described herein, e.g., an antibody chosen from any one of A-H.1 to A-H.85, e.g., A-H.1, A-H.2 or A-H.68, or as described in Table 1, or encoded by the nucleotide sequence in Table 1; or which differs at least 1 or 5 residues, but less than 40, 30, 20, or 10 residues, from a variable region of an antibody described herein.

In some embodiments, the anti-TCRβV antibody molecule, e.g., anti-TCRβ V6 (e.g., anti-TCRβ V6-5*01) antibody molecule, comprises at least one, two, three, or four antigen-binding regions, e.g., variable regions, having an amino acid sequence as set forth in Table 1, or a sequence substantially identical thereto (e.g., a sequence at least about 85%, 90%, 95%, 99%/6 or more identical thereto, or which differs by no more than 1, 2, 5, 10, or 15 amino acid residues from the sequences shown in Table 1. In another embodiment, the anti-TCRβV antibody molecule, e.g., anti-TCRβ V6 (e.g., anti-TCRβ V6-5*01) antibody molecule includes a VH and/or VL domain encoded by a nucleic acid having a nucleotide sequence as set forth in Table 1, or a sequence substantially identical thereto (e.g., a sequence at least about 85%, 90%, 95%, 99% or more identical thereto, or which differs by no more than 3, 6, 15, 30, or 45 nucleotides from the sequences shown in Table 1.

In some embodiments, the anti-TCRβV antibody molecule, e.g., anti-TCRβ V6 (e.g., anti-TCRβ V6-5*01) antibody molecule, comprises: a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO: 9; and/or a VL domain comprising the amino acid sequence of SEQ ID NO: 10, an amino acid sequence at least about 85%, 90%, 95%, 99% or more identical to the amino acid sequence of SEQ ID NO: 10, or an amino acid sequence which differs by no more than 1, 2, 5, 10, or 15 amino acid residues from the amino acid sequence of SEQ ID NO: 10.

In some embodiments, the anti-TCRβV antibody molecule, e.g., anti-TCRβ V6 (e.g., anti-TCRβ V6-5*01) antibody molecule, comprises: a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO: 9; and/or a VL domain comprising the amino acid sequence of SEQ ID NO: 11, an amino acid sequence at least about 85%, 90%, 95%, 99% or more identical to the amino acid sequence of SEQ ID NO: 11, or an amino acid sequence which differs by no more than 1, 2, 5, 10, or 15 amino acid residues from the amino acid sequence of SEQ ID NO: 11.

In some embodiments, the anti-TCRβV antibody molecule, e.g., anti-TCRβ V6 (e.g., anti-TCRβ V6-5*01) antibody molecule is a full antibody or fragment thereof (e.g., a Fab. F(ab′)₂. Fv, single domain antibody, or a single chain Fv fragment (scFv)). In embodiments, the anti-TCRβV antibody molecule. e.g., anti-TCRβ V6 (e.g., anti-TCRβ V6-5*01) antibody molecule is a monoclonal antibody or an antibody with single specificity. In some embodiments, the anti-TCRβV antibody molecule, e.g., anti-TCRβ V6 (e.g., anti-TCRβ V6-5*01) antibody molecule, can also be a humanized, chimeric, camelid, shark, or an in vitro-generated antibody molecule. In some embodiments, the anti-TCRβV antibody molecule, e.g., anti-TCRβ V6 (e.g., anti-TCRβ V6-5*01) antibody molecule, is a humanized antibody molecule. The heavy and light chains of the anti-TCRβV antibody molecule, e.g., anti-TCRβ V6 (e.g., anti-TCRβ V6-5*01) antibody molecule, can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule, e.g., anti-TCRβ V6 (e.g., anti-TCRβ V6-5*0l) antibody molecule, is in the form of a multispecific molecule, e.g., a bispecific molecule, e.g., as described herein.

In some embodiments, the anti-TCRβV antibody molecule, e.g., anti-TCRβ V6 (e.g., anti-TCRβ V6-5*01) antibody molecule, has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1. In some embodiments, the Fc region comprises a Fc region variant, e.g., as described herein.

In some embodiments, the anti-TCRβV antibody molecule, e.g., anti-TCRβ V6 (e.g., anti-TCRβ V6-5*01) antibody molecule, has a light chain constant region chosen from, e.g., the light chain constant regions of kappa or lambda, preferably kappa (e.g., human kappa). In some embodiments, the constant region is altered, e.g., mutated, to modify the properties of the anti-TCRβV antibody molecule, e.g., anti-TCRβ V6 (e.g., anti-TCRβ V6-5*01) antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218), e.g., relative to human IgG1.

Antibody A-H.1 comprises a heavy chain comprising the amino acid sequence of SEQ ID NO: 3278 and a light chain comprising the amino acid sequence of SEQ ID NO: 72. Antibody A-H.2 comprises a heavy chain comprising the amino acid sequence of SEQ ID NO: 3278 and a light chain comprising the amino acid sequence of SEQ ID NO: 3279. Antibody A-H.68 comprises the amino acid sequence of SEQ ID NO: 1337, or a sequence having at least 85%, 90%, 95%, 96%, 97%, 98%, 99%, 99.5%, 99.9%, or 100% sequence identity thereto. Antibody A-H.69 comprises the amino acid sequence of SEQ ID NO: 1500, or a sequence having at least 85%, 90%, 95%, 96%, 97%, 98%, 99%, 99.5%, 99.9%, or 10(0% sequence identity thereto.

Additional exemplary humanized anti-TCRB V6 antibodies are provided in Table 1. In some embodiments, the anti-TCRβ V6 is antibody A, e.g., humanized antibody A (antibody A-H), as provided in Table 1. In some embodiments, the anti-TCRβV antibody comprises one or more (e.g., all three) of a LC CDR 1, LC CDR2, and LC CDR3 provided in Table 1; and/or one or more (e.g., all three) of a HC CDR1, HC CDR2, and HC CDR3 provided in Table 1, or a sequence with at least 85%, 90%, 95%, 96%, 97%, 98%, 99%, 99.5%, 99.9%, or 100% sequence identity thereto. In some embodiments, antibody A comprises a variable heavy chain (VH) and/or a variable light chain (VL) provided in Table 1, or a sequence with at least 85%, 90%, 95%, 96%, 97%, 98%, 99%, 99.5%, 99.9%, or 100% sequence identity thereto.

In some embodiments, the anti-TCRβV antibody molecule, e.g., anti-TCRβ V6 (e.g., anti-TCRβ V6-5*01) antibody molecule comprises a VH of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VL of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VH of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto; and a VL of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

Exemplary anti-TCRβV antibody molecules and the corresponding TCRβV subfamilies recognized by said anti-TCRβV antibody molecules are disclosed in Table 10A.

The various TCRβV subfamilies and/or subfamily members can be expressed at different levels in individuals, e.g., healthy individuals, as disclosed in Kitaura K. et al (2016), BMC Immunology vol 17: 38, the entire contents of which are hereby incorporated by reference. For example, TCRβ V6-5 is represented in approximately 3-6% healthy donors.

The representation of various TCRβV subfamilies and/or subfamily members can also be different in cancer cells. For example, TCRβV is present in about 3-6% of tumor infiltrating T cells irrespective of tumor type (see Li B. et al., Nature Genetics, 2016, vol: 48(7):725-32 the entire contents of which are hereby incorporated by references). Li et al., also disclose that TCRβ V6-5 is present at a high frequency in tumor cells.

### Anti-TCRβ V6 Antibodies

In one aspect, provided herein is an anti-TCRβV antibody molecule that binds to human TCRβ V6, e.g., a TCRβ V6 subfamily comprising: TCRβ V6-4*01, TCRβ V6-4*02, TCRβ 3 V6-9*0I, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments the TCRβ V6 subfamily comprises TCRβ V6-5*0I or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ 3 V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments, TCRβ V6 comprises TCRβ V6-6*01, or a variant thereof. In some embodiments, TCRβ V6 comprises TCRβ V6-2*01, or a variant thereof. In some embodiments, TCRβ V6 comprises TCRβ V6-3*01, or a variant thereof. In some embodiments, TCRβ V6 comprises TCRβ V6-1*01, or a variant thereof.

In some embodiments, TCRβ V6-5*01 is encoded by the nucleic acid sequence of SEQ ID NO: 43, or a sequence having 85%, 90°, 95%, 99% or more identity thereof. In some embodiments, TCRβ V6-5*01 comprises the amino acid sequence of SEQ ID NO: 44, or an amino acid sequence having 85%, 90%, 95%, 99% or more identity thereof.

In some embodiments, the anti-TCRβV antibody molecule, e.g., anti-TCRβ V6 (e.g., anti-TCRβ V6-5*01) antibody molecule, is a non-murine antibody molecule, e.g., a human or humanized antibody molecule. In some embodiments, the anti-TCRβV antibody molecule, e.g., anti-TCRβ V6 (e.g., anti-TCRβ V6-5*01) antibody molecule is a human antibody molecule. In some embodiments, the anti-TCRβV antibody molecule, e.g., anti-TCRβ V6 (e.g., anti-TCRβ V6-5*01) antibody molecule is a humanized antibody molecule.

In some embodiments, the anti-TCRβV antibody molecule, e.g., anti-TCRβ V6 (e.g., anti-TCRβ V6-5*01) antibody molecule, is isolated or recombinant.

In some embodiments, the anti-TCRβV antibody molecule, e.g., anti-TCRβ V6 (e.g., anti-TCRβ V6-5*01) antibody molecule, comprises at least one antigen-binding region, e.g., a variable region or an antigen-binding fragment thereof, from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one, two, three or four variable regions from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one or two heavy chain variable regions from an antibody described herein, e.g., an antibody chosen from any one of A-H.1 to A-H.85, e.g., A-H.1, A-H.2 or A-H.68, or an antibody molecule described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises a heavy chain variable region (VH) having a consensus sequence of SEQ ID NO: 231 or 3290.

-Consensus VH
SEQ ID NO: 231
QVQLVQSGAEVKKPGSSVKVSCKASGH/T/G/YD/T/SFH/R/D/K/TL/D/K/T/NW/F/T/I/Y/GYIHWVR
QAPGQGLEWMGR/WV/I/FF/S/YA/PGSGN/ST/V/Y/IK/RYNEKFKGRVTITADTSTSTAYMELSSLR
SEDTAVYYCAG/VSY/IYSY/AD/GVLDYWGQGTTVTVSS
-Consensus VH
SEQ ID NO: 3290
QVQLVQSGAEVKKPGSSVKVSCKASGX₁X₂FX₃X₄X₅YIHWVRQAPGOGLEWMGX₆X₇X₈X₉GSGX₁₀
X₁₁X₁₂YNEKFKGRVTITADTSTSTAYMELSSLRSEDTAVYYCAX₁₃SX₁₄YSX₁₅X₁₆VLDYWGQGTT
VTVSS, where-in: X₁ is H or T or G or Y; X₂ is D or T or S; X₃ is H or R or D
or K or T; X₄ is L or D or K or T or N; X₅ is W or F or T or I or Y or G; X₆ is
R or W; X₇ is V or I or F; X₈ is F or S or Y; X₉ is A or P; X₁₀ is N or S;
X₁₁ is T or V or Y or I; X₁₂ is K or R; X₁₃ is G or V; X₁₄ is Y or I; X₁₅ is Y or
A; and X₁₆ is D or G.

In some embodiments, the anti-TCRβV antibody molecule, e.g., anti-TCRβ V6 (e.g., anti-TCRβ V6-5*0l) antibody molecule, comprises at least one or two light chain variable regions from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

in some embodiments, the anti-TCRβV antibody molecule comprises a light chain variable region (VL) having a consensus sequence of SEQ ID NO: 230 or 3289.

-Consensus VL
SEQ ID NO: 230
DIQMTQSPSFLSASVGDRVTITCKASQNVG/E/A/DN/DR/KVAWY/HQQKPGKAPKALIYSSSHRY
K/SGVPSRFSGSGSGTEFTLTISSLQPEDFATYFCQQFKSYPLTFGQGTKLEIK
-Consensus VL
SEQ ID NO: 3289
DIQMTQSPSFLSASVGDRVTITCKASQNVX₁X₂X₃VAWX₄QQKPGKAPKALIYSSSHRYX₅GVPSRF
SGSGSGTEFTLTISSLQPEDFATYFCQQFKSYPLTFGQGTKLEIK, wherein X₁ is G, E,
A or D; X₂ is N or D; X₃ is R or K; X₄ is Y or H; and X₅ is K or S

In some embodiments, the anti-TCRβV antibody molecule, e.g., anti-TCRβ V6 (e.g., anti-TCRβ V6-5*01) antibody molecule, comprises a heavy chain constant region for an IgG4, e.g., a human IgG4. In still another embodiment, the anti-TCRβV antibody molecule, e.g., anti-TCRβ V6 (e.g., anti-TCRβ V6-5*01) antibody molecule includes a heavy chain constant region for an IgG1, e.g., a human IgG1. In some embodiments, the heavy chain constant region comprises an amino sequence set forth in Table 3, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes a kappa light chain constant region, e.g., a human kappa light chain constant region. In some embodiments, the light chain constant region comprises an amino sequence set forth in Table 3, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a heavy chain variable region (VH) of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a heavy chain variable region comprising an amino acid sequence shown in Table 1, or encoded by a nucleotide sequence shown in Table 1. In some embodiments, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a light chain variable region of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a light chain variable region comprising an amino acid sequence shown in Table 1, or encoded by a nucleotide sequence shown in Table 1. In some embodiments, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In some embodiments, the anti-TCRβV antibody molecule, e.g., anti-TCRβ V6 (e.g., anti-TCRβ V6-5*01) antibody molecule, includes at least one, two, three, four, five or six CDRs (or collectively all of the CDRs) from a heavy and light chain variable region comprising an amino acid sequence shown in Table 1, or encoded by a nucleotide sequence shown in Table 1. In some embodiments, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In some embodiments, the anti-TCRβV antibody molecule, e.g., anti-TCRβ V6 (e.g., anti-TCRβ V6-5*01) antibody molecule, molecule includes all six CDRs from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in Table 1) from a heavy chain variable region of an antibody described herein, e.g., an antibody chosen from any one of A-H.1 to A-H.85, e.g., A-H.1, A-H.2 or A-H.68, or an antibody described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 1.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in Table 1) from a light chain variable region of an antibody described herein, e.g., an antibody chosen from any one of A-H.1 to A-H.85, e.g., A-H.1, A-H.2 or A-H.68, or an antibody described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 1.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Kabat et al. (e.g., at least one, two, three, four, five, or six CDRs according to the Kabat definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 1.

In some embodiments, the anti-TCRβV antibody molecule, e.g., anti-TCRβ V6 (e.g., anti-TCRβ V6-5*01) antibody molecule, includes all six CDRs according to Kabat et al. (e.g., all six CDRs according to the Kabat definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 1. In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, includes at least one, two, or three hypervariable loops that have the same canonical structures as the corresponding hypervariable loop of an antibody described herein, e.g., an antibody chosen from chosen from any one of A-H.1 to A-H.85, e.g., A-H.1, A-H.2 or A-H.68, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in Table 1) from a heavy chain variable region of an antibody described herein, e.g., an antibody chosen from any one of A-H.1 to A-H.85, e.g., A-H.1, A-H.2 or A-H.68, or as described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 1.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in Table 1) from a light chain variable region of an antibody described herein, e.g., an antibody chosen from any one of A-H.1 to A-H.85, e.g., A-H.1, A-H.2 or A-H.68, or an antibody described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 1.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Chothia et al. (e.g., at least one, two, three, four, five, or six CDRs according to the Chothia definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al. shown in Table 1.

In some embodiments, the anti-TCRβV antibody molecule, e.g., anti-TCRβ V6 (e.g., anti-TCRβ V6-5*01) antibody molecule, includes all six CDRs according to Chothia et al. (e.g., all six CDRs according to the Chothia definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al. shown in Table 1. In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, molecule includes a combination of CDRs or hypervariable loops defined according to Kabat et al., Chothia et al., or as described in Table 1.

In some embodiments, the anti-TCRβV antibody molecule, e.g., anti-TCRβ V6 (e.g., anti-TCRβ V6-5*01) antibody molecule, can contain any combination of CDRs or hypervariable loops according to the Kabat and Chothia definitions.

In some embodiments, a combined CDR as set out in Table 1 is a CDR that comprises a Kabat CDR and a Chothia CDR.

In some embodiments, the anti-TCRβV antibody molecule, e.g., anti-TCRβ V6 (e.g., anti-TCRβ V6-5*01) antibody molecule, molecule includes a combination of CDRs or hypervariable loops identified as combined CDRs in Table 1. In some embodiments, the anti-TCRβV antibody molecule, e.g., anti-TCRβ V6 (e.g., anti-TCRβ V6-5*01) antibody molecule, can contain any combination of CDRs or hypervariable loops according the “combined” CDRs are described in Table 1.

In some embodiments, e.g., an embodiment comprising a variable region, a CDR (e.g., a combined CDR, Chothia CDR or Kabat CDR), or other sequence referred to herein, e.g., in Table 1, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRβV antibody molecule, e.g., anti-TCRβ V6 (e.g., anti-TCRβ V6-5*01) antibody molecule includes: (i) one, two or all of a light chain complementarity determining region 1 (LC CDR1), a light chain complementarity determining region 2 (LC CDR2), and a light chain complementarity determining region 3 (LC CDR3) of SEQ ID NO: 2, SEQ ID NO: 10 or SEQ ID NO: 11, and/or (ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 1 or SEQ ID NO: 9.

In some embodiments, the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 2, and a HC CDR1, HC CDR2, and HC CDR3 of SEQ ID NO: 1.

In some embodiments the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 10, and a HC CDR 1, HC CDR2, and HC CDR3 of SEQ ID NO: 9.

In some embodiments, the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 11, and a HC CDR 1, HC CDR2, and HC CDR3 of SEQ ID NO: 9.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 6, a LC CDR2 amino acid sequence of SEQ ID NO: 7, or a LC CDR3 amino acid sequence of SEQ ID NO: 8; and/or (ii) a HC CDR1 amino acid sequence of SEQ ID NO: 3, a HC CDR2 amino acid sequence of SEQ ID NO: 4, or a HC CDR3 amino acid sequence of SEQ ID NO: 5.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 6, a LC CDR2 amino acid sequence of SEQ ID NO: 7, or a LC CDR3 amino acid sequence of SEQ ID NO: 8; and/or (ii) a heavy chain variable region (VH) comprising a HC CDR1 amino acid sequence of SEQ ID NO: 3, a HC CDR2 amino acid sequence of SEQ ID NO: 4, or a HC CDR3 amino acid sequence of SEQ ID NO: 5.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 51, a LC CDR2 amino acid sequence of SEQ ID NO: 52, or a LC CDR3 amino acid sequence of SEQ ID NO: 53; and/or (ii) a HC CDR1 amino acid sequence of SEQ ID NO: 45, a HC CDR2 amino acid sequence of SEQ ID NO: 46, or a HC CDR3 amino acid sequence of SEQ ID NO: 47.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 51, a LC CDR2 amino acid sequence of SEQ ID NO: 52, or a LC CDR3 amino acid sequence of SEQ ID NO: 53; and/or (ii) a heavy chain variable region (VH) comprising a HC CDR 1 amino acid sequence of SEQ ID NO: 45, a HC CDR2 amino acid sequence of SEQ ID NO: 46, or a HC CDR3 amino acid sequence of SEQ ID NO: 47.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 54, a LC CDR2 amino acid sequence of SEQ ID NO: 55, or a LC CDR3 amino acid sequence of SEQ ID NO: 56; and/or (ii) a HC CDR1 amino acid sequence of SEQ ID NO: 48, a HC CDR2 amino acid sequence of SEQ ID NO: 49, or a HC CDR3 amino acid sequence of SEQ ID NO: 50.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 54, a LC CDR2 amino acid sequence of SEQ ID NO: 55, or a LC CDR3 amino acid sequence of SEQ ID NO: 56; and/or (ii) a heavy chain variable region (VH) comprising a HC CDR1 amino acid sequence of SEQ ID NO: 48, a HC CDR2 amino acid sequence of SEQ ID NO: 49, or a HC CDR3 amino acid sequence of SEQ ID NO: 50.

In some embodiments, the anti-TCRβV antibody molecule, e.g., anti-TCRβ V6 (e.g., anti-TCRβ V6-5*01) antibody molecule comprises a VH and/or a VL of an antibody described in Table 1, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VH and a VL of an antibody described in Table 1, or a sequence with at least 80%, 85%, 90%, 95%, 96%, 97%, 98%, 99% or more identity thereto.

In some embodiments, an anti-TCRVb antibody as described herein has an antigen binding domain having a VL having a consensus sequence of SEQ ID NO: 230, wherein position 30 is G, E, A or D; position 31 is N or D; position 32 is R or K: position 36 is Y or H; and/or position 56 is K or S.

In some embodiments, an anti-TCRVb antibody as described herein has an antigen binding domain having a VH having a consensus sequence of SEQ ID NO: 231, wherein: position 27 is H or T or G or Y; position 28 is D or T or S; position 30 is H or R or D or K or T; position 31 is L or D or K or T or N; position 32 is W or F or T or I or Y or G; position 49 is R or W; position 50 is V or I or F; position 51 is F or S or Y; position 52 is A or P; position 56 is N or S; position 57 is T or V or Y or I; position 58 is K or R; position 97 is G or V; position 99 is Y or I; position 102 is Y or A; and/or position 103 is D or G.

### Anti-TCRβV12 Antibodies

In one aspect, provided herein is an anti-TCRβV antibody molecule that binds to human TCRβ V12, e.g., a TCRβ V12 subfamily comprising: TCRβ V12-4*01, TCRβ V12-3*01 or TCRβ V12-5*01. In some embodiments the TCRβ V12 subfamily comprises TCRβ V12-4*01. In some embodiments the TCRβ V12 subfamily comprises TCRβ V12-3*01.

In some embodiments, the anti-TCRβV antibody molecule, e.g., anti-TCRβ V12 antibody molecule, is a non-murine antibody molecule, e.g., a human or humanized antibody molecule. In some embodiments, the anti-TCRβV antibody molecule, e.g., anti-TCRβ V12 antibody molecule is a human antibody molecule. In some embodiments, the anti-TCRβV antibody molecule, e.g., anti-TCRβ V12 antibody molecule is a humanized antibody molecule.

In some embodiments, the anti-TCRβV antibody molecule, e.g., anti-TCRβ V12 antibody molecule, is isolated or recombinant.

In some embodiments, the anti-TCRβV antibody molecule, e.g., anti-TCRβ V12 antibody molecule, comprises at least one antigen-binding region, e.g., a variable region or an antigen-binding fragment thereof, from an antibody described herein, e.g., an antibody described in Table 2, or encoded by a nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises at least one, two, three or four variable regions from an antibody described herein. e.g., an antibody as described in Table 2, or encoded by a nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises at least one or two heavy chain variable regions from an antibody described herein, e.g., an antibody as described in Table 2, or encoded by a nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises at least one or two light chain variable regions from an antibody described herein. e.g., an antibody as described in Table 2, or encoded by a nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises a heavy chain constant region for an IgG4, e.g., a human IgG4. In still another embodiment, the anti-TCRβV antibody molecule, e.g., anti-TCRβ V12 antibody molecule, includes a heavy chain constant region for an IgG1, e.g., a human IgG1. In some embodiments, the heavy chain constant region comprises an amino sequence set forth in Table 3, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V12 antibody molecule, includes a kappa light chain constant region, e.g., a human kappa light chain constant region. In some embodiments, the light chain constant region comprises an amino sequence set forth in Table 3, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V12 antibody molecule, includes at least one, two, or three complementarity determining regions (CDRs) from a heavy chain variable region of an antibody described herein, e.g., an antibody as described in Table 2, or encoded by the nucleotide sequence in Table 2, or a sequence substantially identical (e.g., at least 80%, 85%, 90)%, 92%, 95%, 97%, 98%, 99% or higher identical) to any of the aforesaid sequences.

In some embodiments, the anti-TCRβV antibody molecule, e.g., anti-TCRβ V12 antibody molecule, includes at least one, two, or three CDRs (or collectively all of the CDRs) from a heavy chain variable region comprising an amino acid sequence shown in Table 2, or encoded by a nucleotide sequence shown in Table 2. In some embodiments, one or more of the CDRs (or collectively all of the CDRs) have one, two, three, four, five, six or more changes, e.g., amino acid substitutions or deletions, relative to the amino acid sequence shown in Table 2, or encoded by a nucleotide sequence shown in Table 2.

In some embodiments, the anti-TCRβV antibody molecule, e.g., anti-TCRβ V12 antibody molecule, includes at least one, two, or three complementarity determining regions (CDRs) from a light chain variable region of an antibody described herein, e.g., an antibody as described in Table 2, or encoded by the nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, includes at least one, two, or three CDRs (or collectively all of the CDRs) from a light chain variable region comprising an amino acid sequence shown in Table 2, or encoded by a nucleotide sequence shown in Table 2. In some embodiments, one or more of the CDRs (or collectively all of the CDRs) have one, two, three, four, five, six or more changes, e.g., amino acid substitutions or deletions, relative to the amino acid sequence shown in Table 2, or encoded by a nucleotide sequence shown in Table 2.

In some embodiments, the anti-TCRβV antibody molecule, e.g., anti-TCRβ V12 antibody molecule, includes at least one, two, three, four, five or six CDRs (or collectively all of the CDRs) from a heavy and light chain variable region comprising an amino acid sequence shown in Table 2, or encoded by a nucleotide sequence shown in Table 2. In some embodiments, one or more of the CDRs (or collectively all of the CDRs) have one, two, three, four, five, six or more changes, e.g., amino acid substitutions or deletions, relative to the amino acid sequence shown in Table 2, or encoded by a nucleotide sequence shown in Table 2.

In some embodiments, the anti-TCRβV antibody molecule, e.g., anti-TCRβ V12 antibody molecule, molecule includes all six CDRs from an antibody described herein, e.g., an antibody as described in Table 2, or encoded by the nucleotide sequence in Table 2, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule, e.g., anti-TCRβ V12 antibody molecule, may include any CDR described herein.

In some embodiments, the anti-TCRβV antibody molecule, e.g., anti-TCRβ V12 antibody molecule includes at least one, two, or three CDRs according to Kabat et al. (e.g., at least one, two, or three CDRs according to the Kabat definition as set out in Table 2) from a heavy chain variable region of an antibody described herein, e.g., an antibody chosen as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 2.

In some embodiments, the anti-TCRβV antibody molecule, e.g., anti-TCRβ V12 antibody molecule includes at least one, two, or three CDRs according to Kabat et al. (e.g., at least one, two, or three CDRs according to the Kabat definition as set out in Table 2) from a light chain variable region of an antibody described herein, e.g., an antibody as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 2.

In some embodiments, the anti-TCRβV antibody molecule, e.g., anti-TCRβ V12 antibody molecule includes at least one, two, three, four, five, or six CDRs according to Kabat et al. (e.g., at least one, two, three, four, five, or six CDRs according to the Kabat definition as set out in Table 2) from the heavy and light chain variable regions of an antibody described herein, e.g., an antibody as described in Table 2, or encoded by the nucleotide sequence in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 2.

In some embodiments, the anti-TCRβV antibody molecule, e.g., anti-TCRβ V12 antibody molecule includes all six CDRs according to Kabat et al. (e.g., all six CDRs according to the Kabat definition as set out in Table 2) from the heavy and light chain variable regions of an antibody described herein, e.g., an antibody as described in Table 2, or encoded by the nucleotide sequence in Table 2; or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 2. In some embodiments, the anti-TCRβV antibody molecule, e.g., anti-TCRβ V12 antibody molecule may include any CDR described herein.

In some embodiments, the anti-TCRβV antibody molecule, e.g., anti-TCRβ V12 antibody molecule includes at least one, two, or three hypervariable loops that have the same canonical structures as the corresponding hypervariable loop of an antibody described herein, e.g., an antibody described in Table 2, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβ V12 antibody molecule includes at least one, two, or three CDRs according to Chothia et al. (e.g., at least one, two, or three CDRs according to the Chothia definition as set out in Table 2) from a heavy chain variable region of an antibody described herein, e.g., an antibody chosen as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 2.

In some embodiments, the anti-TCRβV antibody molecule, e.g., anti-TCRβ V12 antibody molecule includes at least one, two, or three CDRs according to Chothia et al. (e.g., at least one, two, or three CDRs according to the Chothia definition as set out in Table 2) from a light chain variable region of an antibody described herein, e.g., an antibody as described in Table 2, or a sequence substantially identical (e.g., at least 80%, 85%, 90%, 92%, 95%, 97%, 98%, 99%/o or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 2.

In some embodiments, the anti-TCRβV antibody molecule, e.g., anti-TCRβ V12 antibody molecule includes at least one, two, three, four, five, or six CDRs according to Chothia et al. (e.g., at least one, two, three, four, five, or six CDRs according to the Chothia definition as set out in Table 2) from the heavy and light chain variable regions of an antibody described herein, e.g., an antibody as described in Table 2, or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al. shown in Table 2.

In some embodiments, the anti-TCRβV antibody molecule, e.g., anti-TCRβ V12 antibody molecule includes all six CDRs according to Chothia et al. (e.g., all six CDRs according to the Chothia definition as set out in Table 2) from the heavy and light chain variable regions of an antibody described herein, e.g., an antibody as described in Table 2, or encoded by the nucleotide sequence in Table 2; or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al. shown in Table 2. In some embodiments, the anti-TCRβV antibody molecule, e.g., anti-TCRβ V12 antibody molecule may include any CDR described herein.

In some embodiments, the anti-TCRβV antibody molecule, e.g., anti-TCRβ V12 antibody molecule includes at least one, two, or three CDRs according to a combined CDR (e.g., at least one, two, or three CDRs according to the combined CDR definition as set out in Table 2) from a heavy chain variable region of an antibody described herein, e.g., an antibody chosen as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to combined CDR shown in Table 2.

In some embodiments, the anti-TCRβV antibody molecule, e.g., anti-TCRβ V12 antibody molecule includes at least one, two, or three CDRs according to a combined CDR (e.g., at least one, two, or three CDRs according to the combined CDR definition as set out in Table 2) from a light chain variable region of an antibody described herein, e.g., an antibody as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to a combined CDR shown in Table 2.

In some embodiments, the anti-TCRβV antibody molecule, e.g., anti-TCRβ V12 antibody molecule includes at least one, two, three, four, five, or six CDRs according to a combined CDR. (e.g., at least one, two, three, four, five, or six CDRs according to the combined CDR definition as set out in Table 2) from the heavy and light chain variable regions of an antibody described herein, e.g., an antibody as described in Table 2, or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to a combined CDR shown in Table 2.

In some embodiments, the anti-TCRβV antibody molecule, e.g., anti-TCRβ V12 antibody molecule includes all six CDRs according to a combined CDR (e.g., all six CDRs according to the combined CDR definition as set out in Table 2) from the heavy and light chain variable regions of an antibody described herein, e.g., an antibody as described in Table 2, or encoded by the nucleotide sequence in Table 2; or encoded by the nucleotide sequence in Table 2; or a sequence substantially identical (e.g., at least 80%, 85%, 0%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to a combined CDR shown in Table 2. In some embodiments, the anti-TCRβV antibody molecule, e.g., anti-TCRβ V12 antibody molecule may include any CDR described herein.

In some embodiments, a combined CDR as set out in Table 1 is a CDR that comprises a Kabat CDR and a Chothia CDR.

In some embodiments, the anti-TCRβV antibody molecule, e e.g., anti-TCRβ V12 antibody molecule, molecule includes a combination of CDRs or hypervariable loops identified as combined CDRs in Table 1. In some embodiments, the anti-TCRβV antibody molecule, e.g., anti-TCRβ V12 antibody molecule, can contain any combination of CDRs or hypervariable loops according the “combined” CDRs are described in Table 1.

In some embodiments, the anti-TCRβV antibody molecule, e.g., anti-TCRβ V12 antibody molecule includes a combination of CDRs or hypervariable loops defined according to the Kabat et al, and Chothia et al., or as described in Table 1.

In some embodiments, the anti-TCRβV antibody molecule, e.g., anti-TCRβ V12 antibody molecule can contain any combination of CDRs or hypervariable loops according to the Kabat and Chothia definitions.

In some embodiments, e.g., an embodiment comprising a variable region, a CDR (e.g., a combined CDR, Chothia CDR or Kabat CDR), or other sequence referred to herein, e.g., in Table 2, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRβV antibody molecule, e.g., anti-TCRβ V12 antibody molecule includes: (i) one, two or all of a light chain complementarity determining region 1 (LC CDR1), a light chain complementarity determining region 2 (LC CDR2), and a light chain complementarity determining region 3 (LC CDR3) of SEQ ID NO: 16, SEQ ID NO: 26, SEQ ID NO: 27, SEQ ID NO: 28, SEQ ID NO: 29 or SEQ ID NO: 30, and/or (ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 15, SEQ ID NO: 23. SEQ ID NO: 24 or SEQ ID NO: 25.

In some embodiments, the anti-TCRβV antibody molecule, e.g., anti-TCRβ V12 antibody molecule comprises: (i) a LC CDR1 amino acid sequence of SEQ ID NO: 20, a LC CDR2 amino acid sequence of SEQ ID NO: 21, or a LC CDR3 amino acid sequence of SEQ ID NO: 22; and/or (ii) a HC CDR1 amino acid sequence of SEQ ID NO: 17, a HC CDR2 amino acid sequence of SEQ ID NO: 18, or a HC CDR3 amino acid sequence of SEQ ID NO: 19.

In some embodiments, the anti-TCRβV antibody molecule, e.g., anti-TCRβ V12 antibody molecule comprises: (i) a light chain variable region (VL) comprising a LC CDR1 amino acid sequence of SEQ ID NO: 20, a LC CDR2 amino acid sequence of SEQ ID NO: 21, and a LC CDR3 amino acid sequence of SEQ ID NO: 2; and/or (ii) a heavy chain variable region (VH) comprising a HC CDR1 amino acid sequence of SEQ ID NO: 17, a HC CDR2 amino acid sequence of SEQ ID NO: 18, and a HC CDR3 amino acid sequence of SEQ ID NO: 19.

In some embodiments, the anti-TCRβV antibody molecule, e.g., anti-TCRβ V12 antibody molecule comprises: (i) a LC CDR1 amino acid sequence of SEQ ID NO: 63, a LC CDR2 amino acid sequence of SEQ ID NO: 64, or a LC CDR3 amino acid sequence of SEQ ID NO: 65; and/or (ii) a HC CDR1 amino acid sequence of SEQ ID NO: 57, a HC CDR2 amino acid sequence of SEQ ID NO: 58, or a HC CDR3 amino acid sequence of SEQ ID NO: 59.

In some embodiments, the anti-TCRβV antibody molecule, e.g., anti-TCRβ V12 antibody molecule comprises: (i) a light chain variable region (VL) comprising a LC CDR1 amino acid sequence of SEQ ID NO: 63, a LC CDR2 amino acid sequence of SEQ ID NO: 64, or a LC CDR3 amino acid sequence of SEQ ID NO: 65; and/or (ii) a heavy chain variable region (VH) comprising a HC CDR1 amino acid sequence of SEQ ID NO: 57, a HC CDR2 amino acid sequence of SEQ ID NO: 58, or a HC CDR3 amino acid sequence of SEQ ID NO: 59.

In some embodiments, the anti-TCRβV antibody molecule, e.g., anti-TCRβ V12 antibody molecule comprises: (i) a LC CDR1 amino acid sequence of SEQ ID NO: 66, a LC CDR2 amino acid sequence of SEQ ID NO: 67, or a LC CDR3 amino acid sequence of SEQ ID NO: 68; and/or (ii) a HC CDR1 amino acid sequence of SEQ ID NO: 60, a HC CDR2 amino acid sequence of SEQ ID NO: 61, or a HC CDR3 amino acid sequence of SEQ ID NO: 62.

In some embodiments, the anti-TCRβV antibody molecule, e.g., anti-TCRβ V12 antibody molecule comprises: (i) a light chain variable region (VL) comprising a LC CDR1 amino acid sequence of SEQ ID NO: 63, a LC CDR2 amino acid sequence of SEQ ID NO: 64, or a LC CDR3 amino acid sequence of SEQ ID NO: 65; and/or (ii) a heavy chain variable region (VH) comprising a HC CDR 1 amino acid sequence of SEQ ID NO: 57, a HC CDR2 amino acid sequence of SEQ ID NO: 58, or a HC CDR3 amino acid sequence of SEQ ID NO: 59.

In some embodiments, the light or the heavy chain variable framework (e.g., the region encompassing at least FR1, FR2, FR3, and optionally FR4) of the anti-TCRβV antibody molecule, e.g., anti-TCRβ V12 antibody molecule can be chosen from: (a) a light or heavy chain variable framework including at least 80%, 85%, 87% 90%, 92%, 93%, 95%, 97%, 98%, or 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In some embodiments, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

In some embodiments, the anti-TCRβV antibody molecule, e.g., anti-TCRβ V12 antibody molecule, comprises a heavy chain variable domain having at least one, two, three, four, five, six, seven, ten, fifteen, twenty or more changes, e.g., amino acid substitutions or deletions, from an amino acid sequence described in Table 2, e.g., the amino acid sequence of the FR region in the entire variable region, e.g., shown in FIGS. 17A and 17B, or in SEQ ID NOs: 23-25.

Alternatively, or in combination with the heavy chain substitutions described herein the anti-TCRβV antibody molecule, e.g., anti-TCRβ V12 antibody molecule comprises a light chain variable domain having at least one, two, three, four, five, six, seven, ten, fifteen, twenty or more amino acid changes, e.g., amino acid substitutions or deletions, from an amino acid sequence of an antibody described herein, e.g., the amino acid sequence of the FR region in the entire variable region, e.g., shown in FIGS. 17A and 17B, or in SEQ ID NOs: 26-30.

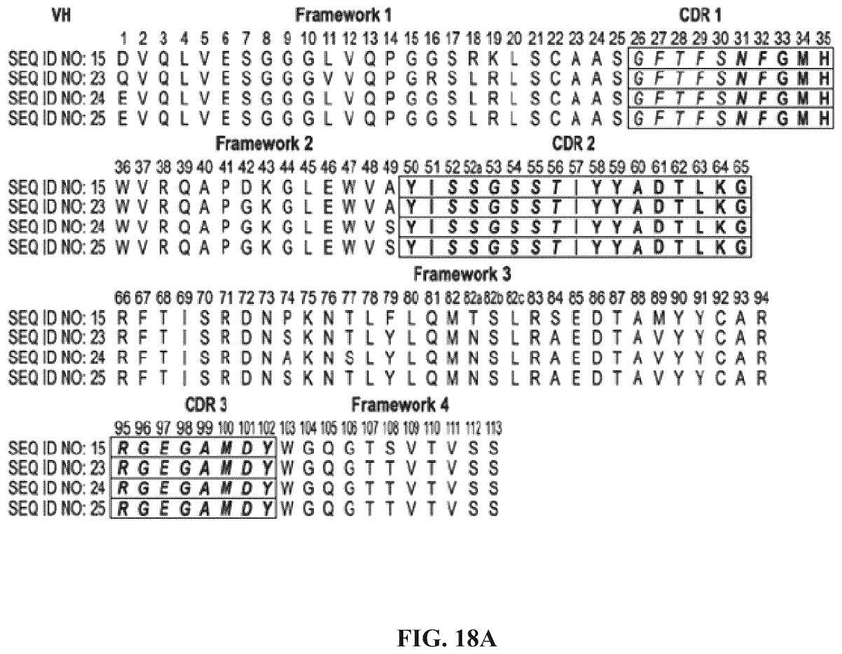

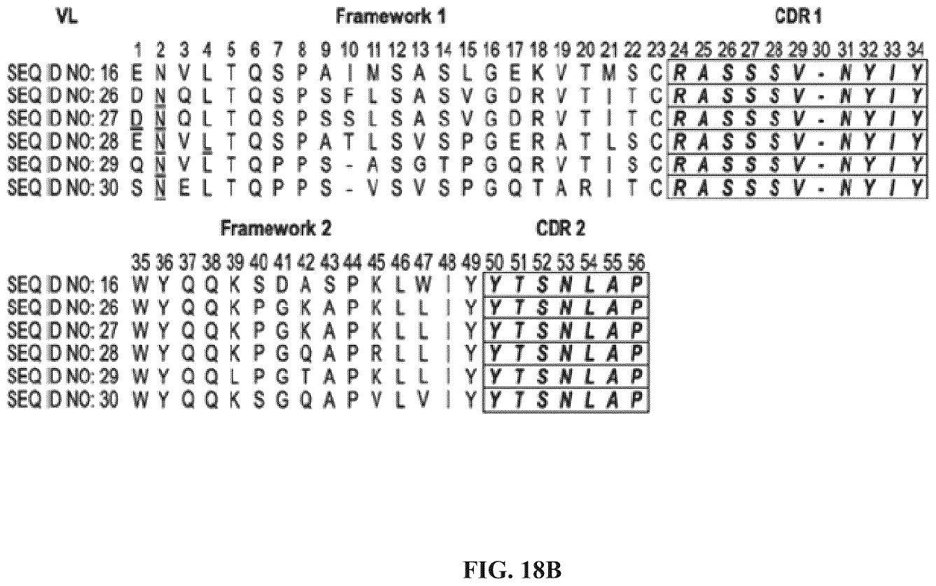

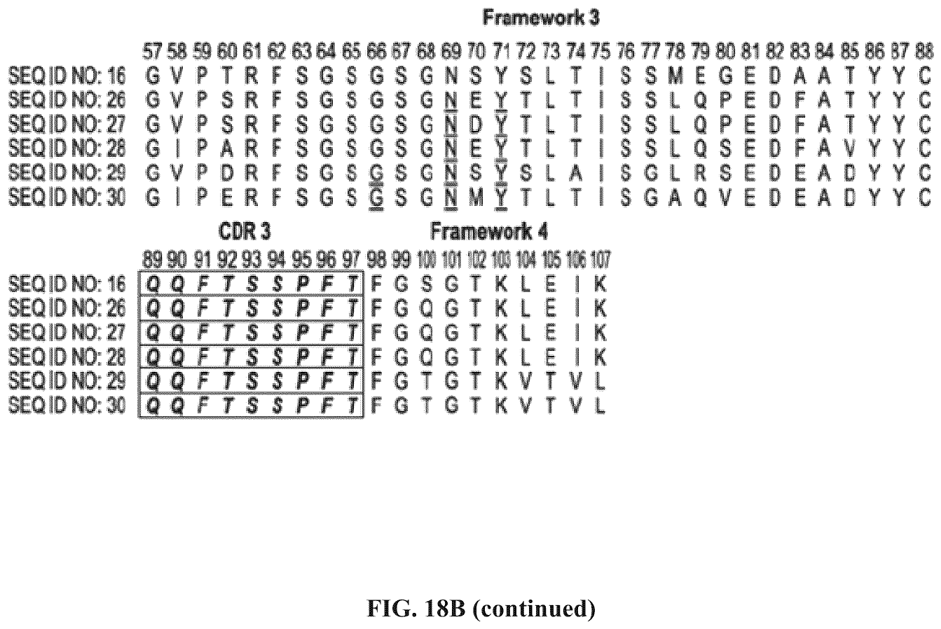

In some embodiments, the anti-TCRβV antibody molecule, e.g., anti-TCRβ V12 antibody molecule includes one, two, three, or four heavy chain framework regions shown in FIG. 18A, or a sequence substantially identical thereto.

In some embodiments, the anti-TCRβV antibody molecule, e.g., anti-TCRβ V12 antibody molecule includes one, two, three, or four light chain framework regions shown in FIG. 18B, or a sequence substantially identical thereto. In some embodiments, the anti-TCRβV antibody molecule, e.g., anti-TCRβ V12 antibody molecule comprises the light chain framework region 1 e.g., as shown in FIG. 18B. In some embodiments, the anti-TCRβV antibody molecule, e.g., anti-TCRβ V12 antibody molecule comprises the light chain framework region 2 e.g., as shown in FIG. 18B. In some embodiments, the anti-TCRβV antibody molecule, e.g., anti-TCRβ V12 antibody molecule comprises the light chain framework region 3, e.g., as shown in FIG. 18B. In some embodiments, the anti-TCRβV antibody molecule, e.g., anti-TCRβ V12 antibody molecule comprises the light chain framework region 4, e.g., as shown in FIG. 18B.

In some embodiments, the anti-TCRβV antibody molecule, e.g., anti-TCRβ V12 antibody molecule comprises a light chain comprising a framework region, e.g., framework region 1 (FR1), comprising a change, e.g., a substitution (e.g., a conservative substitution) at one or more, e.g., all, position as described herein according to Kabat numbering. In some embodiments, FR1 comprises an Aspartic Acid at position 1, e.g., a substitution at position 1 according to Kabat numbering, e.g., an Alanine to Aspartic Acid substitution. In some embodiments, FR1 comprises an Asparagine at position 2. e.g., a substitution at position 2 according to Kabat numbering, e.g., an Isoleucine to Asparagine substitution, Serine to Asparagine substitution or Tyrosine to Asparagine substitution. In some embodiments, FR1 comprises a Leucine at position 4, e.g., a substitution at position 4 according to Kabat numbering, e.g., a Methionine to Leucine substitution.

In some embodiments, the anti-TCRβV antibody molecule, e.g., anti-TCRβ V12 antibody molecule comprises a light chain comprising a framework region, e.g., framework region 1 (FR1), comprising a substitution at position 1 according to Kabat numbering, e.g., an Alanine to Aspartic Acid substitution, a substitution at position 2 according to Kabat numbering, e.g., an Isoleucine to Asparagine substitution, Serine to Asparagine substitution or Tyrosine to Asparagine substitution, and a substitution at position 4 according to Kabat numbering, e.g., a Methionine to Leucine substitution. In some embodiments, the anti-TCRβV antibody molecule, e.g., anti-TCRβ V12 antibody molecule comprises a light chain comprising a framework region, e.g., framework region 1 (FR1), comprising a substitution at position 1 according to Kabat numbering, e.g., an Alanine to Aspartic Acid substitution, and a substitution at position 2 according to Kabat numbering, e.g., an Isoleucine to Asparagine substitution. Serine to Asparagine substitution or Tyrosine to Asparagine substitution. In some embodiments, the anti-TCRβV antibody molecule, e.g., anti-TCRβ V12 antibody molecule comprises a light chain comprising a framework region, e.g., framework region 1 (FR1), comprising a substitution at position 1 according to Kabat numbering, e.g., an Alanine to Aspartic Acid substitution, and a substitution at position 4 according to Kabat numbering, e.g., a Methionine to Leucine substitution. In some embodiments, the anti-TCRβV antibody molecule, e.g., anti-TCRβ V12 antibody molecule comprises a light chain comprising a framework region, e.g., framework region 1 (FR1), comprising a substitution at position 2 according to Kabat numbering, e.g., an Isoleucine to Asparagine substitution, Serine to Asparagine substitution or Tyrosine to Asparagine substitution, and a substitution at position 4 according to Kabat numbering, e.g., a Methionine to Leucine substitution. In some embodiments, the substitution is relative to a human germline light chain framework region sequence.

In some embodiments, the anti-TCRβV antibody molecule, e.g., anti-TCRβ V12 antibody molecule comprises a light chain comprising a framework region, e.g., framework region 3 (FR3), comprising a change, e.g., a substitution (e.g., a conservative substitution) at one or more, e.g., all, position as described herein according to Kabat numbering. In some embodiments, FR3 comprises a Glycine at position 66, e.g., a substitution at position 66 according to Kabat numbering, e.g., a Lysine to Glycine substitution, or a Serine to Glycine substitution. In some embodiments, FR3 comprises an Asparagine at position 69, e.g., a substitution at position 69 according to Kabat numbering, e.g., a Tyrosine to Asparagine substitution. In some embodiments. FR3 comprises a Tyrosine at position 71, e.g., a substitution at position 71 according to Kabat numbering, e.g., a Phenylalanine to Tyrosine substitution, or an Alanine to Tyrosine substitution.

In some embodiments, the anti-TCRβV antibody molecule, e.g., anti-TCRβ V12 antibody molecule comprises a light chain comprising a framework region, e.g., framework region 3 (FR3), comprising a substitution at position 66 according to Kabat numbering, e.g., a Lysine to Glycine substitution, or a Serine to Glycine substitution, and a substitution at position 69 according to Kabat numbering, e.g., a Tyrosine to Asparagine substitution. In some embodiments, the anti-TCRβV antibody molecule, e.g., anti-TCRβ V12 antibody molecule comprises a light chain comprising a framework region, e.g., framework region 3 (FR3), comprising a substitution at position 66 according to Kabat numbering, e.g., Lysine to Glycine substitution, or a Serine to Glycine substitution, and a substitution at position 71 according to Kabat numbering, e.g., a Phenylalanine to Tyrosine substitution, or an Alanine to Tyrosine substitution. In some embodiments, the anti-TCRβV antibody molecule, e.g., anti-TCRβ V12 antibody molecule comprises a light chain comprising a framework region, e.g., framework region 3 (FR3), comprising a substitution at position 69 according to Kabat numbering, e.g., a Tyrosine to Asparagine substitution and a substitution at position 71 according to Kabat numbering, e.g., a Phenylalanine to Tyrosine substitution, or an Alanine to Tyrosine substitution. In some embodiments, the anti-TCRβV antibody molecule, e.g., anti-TCRβ V12 antibody molecule comprises a light chain comprising a framework region, e.g., framework region 3 (FR3), comprising a substitution at position 66 according to Kabat numbering, e.g., a Lysine to Glycine substitution, or a Serine to Glycine substitution, a substitution at position 69 according to Kabat numbering, e.g., a Tyrosine to Asparagine substitution and a substitution at position 71 according to Kabat numbering, e.g., a Phenylalanine to Tyrosine substitution, or an Alanine to Tyrosine substitution. In some embodiments, the substitution is relative to a human germline light chain framework region sequence.

In some embodiments, the anti-TCRβV antibody molecule, e.g., anti-TCRβ V12 antibody molecule comprises a light chain comprising: a framework region 1 (FR 1) comprising a substitution at position 2 according to Kabat numbering, e.g., a Isoleucine to Asparagine substitution; and a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 26. In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 1 according to Kabat numbering, e.g., a Alanine to Aspartic Acid substitution, and a substitution at position 2 according to Kabat numbering, e.g., a Isoleucine to Asparagine substitution; and (b) a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 27 In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 2 according to Kabat numbering, e.g., a Serine to Asparagine substitution; and a substitution at position 4 according to Kabat numbering, e.g., a Methionine to Leucine substitution; and (b) a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 28 In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 2 according to Kabat numbering, e.g., a Serine to Asparagine substitution; and (b) a framework region 3 (FR3) comprising a substitution at position 66 according to Kabat numbering, e.g., a Lysine to Glycine substitution: a substitution at position 69 according to Kabat numbering, e.g., a Threonine to Asparagine substitution; and a substitution at position 71 according to Kabat numbering, e.g., a Alanine to Tyrosine substitution, e.g., as shown in the amino acid sequence of SEQ ID NO: 29. In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 2 according to Kabat numbering, e.g., a Tyrosine to Asparagine substitution; and (b) a framework region 3 (FR3) comprising a substitution at position 66 according to Kabat numbering, e.g., a Serine to Glycine substitution: a substitution at position 69 according to Kabat numbering, e.g., a Threonine to Asparagine substitution; and a substitution at position 71 according to Kabat numbering, e.g., a Alanine to Tyrosine substitution, e.g., as shown in the amino acid sequence of SEQ ID NO: 29. In some embodiments, the substitution is relative to a human germline light chain framework region sequence.

In some embodiments, the anti-TCRβV antibody molecule, e.g., anti-TCRβ V12 antibody molecule comprises a light chain variable domain comprising: (a) a framework region 1 (FR1) comprising a change, e.g., a substitution (e.g., a conservative substitution) at one or more (e.g., all) positions as described herein according to Kabat numbering, and (b) a framework region 3 (FR3) comprising a change, e.g., a substitution (e.g., a conservative substitution) at one or more (e.g., all) position as described herein according to Kabat numbering. In some embodiments, the substitution is relative to a human germline light chain framework region sequence.

In some embodiments, the anti-TCRβV antibody molecule, e.g., anti-TCRβ V12 antibody molecule comprises the heavy chain framework region 1, e.g., as shown in FIG. 18A. In some embodiments, the anti-TCRβV antibody molecule, e.g., anti-TCRβ V12 antibody molecule comprises the heavy chain framework region 2, e.g., as shown in FIG. 18A. In some embodiments, the anti-TCRβV antibody molecule, e.g., anti-TCRβ V12 antibody molecule comprises the heavy chain framework region 3, e.g., as shown in FIG. 18A. In some embodiments, the anti-TCRβV antibody molecule, e.g., anti-TCRβ V12 antibody molecule comprises the heavy chain framework region 4, e.g., as shown in FIG. 18A. In some embodiments, the anti-TCRβV antibody molecule, e.g., anti-TCRβ V12 antibody molecule comprises the heavy chain framework regions 1-4, e.g., SEQ ID NOS: 20-23, or as shown in FIG. 18A. In some embodiments, the anti-TCRβV antibody molecule, e.g., anti-TCRβ V12 antibody molecule comprises the light chain framework regions 1-4, e.g., SEQ ID NOs: 26-30, or as shown in FIG. 18B.

In some embodiments, the anti-TCRβV antibody molecule, e.g., anti-TCRβ V12 antibody molecule comprises the heavy chain framework regions 1-4, e.g., SEQ ID NOs: 23-25; and the light chain framework regions 1-4, e.g., SEQ ID NOs: 26-30, or as shown in FIGS. 17A and 17B.

In some embodiments, the heavy or light chain variable domain, or both, of, the anti-TCRβV antibody molecule, e.g., anti-TCRβ V12 antibody molecule includes an amino acid sequence, which is substantially identical to an amino acid as described herein, e.g., at least 80%, 85%, 90%, 92%, 95%, 97%, 98%, 99% or higher identical to a variable region of an antibody described herein, e.g., an antibody as described in Table 2, or encoded by the nucleotide sequence in Table 2, or which differs at least 1 or 5 residues, but less than 40, 30, 20, or 10 residues, from a variable region of an antibody described herein.

In some embodiments, the anti-TCRβV antibody molecule, e.g., anti-TCRβ V12 antibody molecule comprises at least one, two, three, or four antigen-binding regions, e.g., variable regions, having an amino acid sequence as set forth in Table 2, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 2. In another embodiment, the anti-TCRβV antibody molecule, e.g., anti-TCRβ V12 antibody molecule includes a VH and/or VL domain encoded by a nucleic acid having a nucleotide sequence as set forth in Table 2, or a sequence substantially identical thereto (e.g., a sequence at least about 85%, 90%, 95%, 99% or more identical thereto, or which differs by no more than 3, 6, 15, 30, or 45 nucleotides from the sequences shown in Table 2.

In some embodiments, the anti-TCRβV antibody molecule, e.g., anti-TCRβ V12 antibody molecule comprises: a VH domain comprising an amino acid sequence chosen from the amino acid sequence of SEQ ID NO: 23, SEQ ID NO:24 or SEQ ID NO:25, an amino acid sequence at least about 85%, 90%, 95%, 99% or more identical to the amino acid sequence SEQ ID NO: 23, SEQ ID NO:24 or SEQ ID NO:25, or an amino acid sequence which differs by no more than 1, 2, 5, 10, or 15 amino acid residues from the amino acid sequence of SEQ ID NO: 23, SEQ ID NO:24 or SEQ ID NO:25; and/or a VL domain comprising an amino acid sequence chosen from the amino acid sequence of SEQ ID NO: 26. SEQ ID NO: 27, SEQ ID NO: 28, SEQ ID NO: 29 or SEQ ID NO: 30, an amino acid sequence at least about 85%, 90%, 95%, 99% or more identical to the amino acid sequence of SEQ ID NO: 26, SEQ ID NO: 27, SEQ ID NO: 28, SEQ ID NO: 29 or SEQ ID NO: 30, or an amino acid sequence which differs by no more than 1, 2, 5, 10, or 15 amino acid residues from the amino acid sequence of SEQ ID NO: 26, SEQ ID NO: 27, SEQ ID NO: 28, SEQ ID NO: 29 or SEQ ID NO: 30. In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 26, an amino acid sequence at least about 85%, 90%, 95%, 99°/o or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβ V12 antibody molecule is a full antibody or fragment thereof (e.g., a Fab. F(ab)₂. Fv, or a single chain Fv fragment (scFv)). In embodiments, the anti-TCRβV antibody molecule, e.g., anti-TCRβ V6 (e.g., anti-TCRβ V6-5*01) antibody molecule is a monoclonal antibody or an antibody with single specificity. In some embodiments, the anti-TCRβV antibody molecule, e.g., anti-TCRβ V12 antibody molecule, can also be a humanized, chimeric, camelid, shark, or an in vitro-generated antibody molecule. In some embodiments, the anti-TCRβV antibody molecule, e.g., anti-TCRβ V12 antibody molecule is a humanized antibody molecule. The heavy and light chains of the anti-TCRβV antibody molecule, e.g., anti-TCRβ V12 antibody molecule can be full-length (e.g., an antibody can include at least one, and preferably two, complete heavy chains, and at least one, and preferably two, complete light chains) or can include an antigen-binding fragment (e.g., a Fab. F(ab′)2. Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule, e.g., anti-TCRβ V12 antibody molecule is in the form of a multispecific molecule, e.g., a bispecific molecule, e.g., as described herein.

In some embodiments, the anti-TCRβV antibody molecule, e.g., anti-TCRβ V12 antibody molecule has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1.

In some embodiments, the anti-TCRβV antibody molecule, e.g., anti-TCRβ V12 antibody molecule has a light chain constant region chosen from, e.g., the light chain constant regions of kappa or lambda, preferably kappa (e.g., human kappa). In some embodiments, the constant region is altered, e.g., mutated, to modify the properties of the anti-TCRβV antibody molecule, e.g., anti-TCRβ V12 antibody molecule (e.g., to increase or decrease one or more of: Fc receptor binding, antibody glycosylation, the number of cysteine residues, effector cell function, or complement function). For example, the constant region is mutated at positions 2% (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218).

Antibody B-H.1 comprises a first chain comprising the amino acid sequence of SEQ ID NO: 3280 and a second chain comprising the amino acid sequence of SEQ ID NO: 3281.

Additional exemplary anti-TCRβ V12 antibodies are provided in Table 2. In some embodiments, the anti-TCRβ V12 is antibody B, e.g., humanized antibody B (antibody B-H), as provided in Table 2. In some embodiments, the anti-TCRβV antibody comprises one or more (e.g., all three) of a LC CDR1, LC CDR2, and LC CDR3 provided in Table 2; and/or one or more (e.g., all three) of a HC CDR1, HC CDR2, and HC CDR3 provided in Table 2, or a sequence with at least 95% sequence identity thereto. In some embodiments, antibody B comprises a variable heavy chain (VH) and/or a variable light chain (VL) provided in Table 2, or a sequence with at least 95% sequence identity thereto.

In some embodiments, the anti-TCRβVB 12 antibody molecule (e.g., anti-TCRβVB 12-3 or anti-TCRVB 12-4 antibody molecule) comprises a VH of B-H.1A, B-H.1B, B-H.1C, B-H.1D, B-H.1E. B-H.1F, B-H.1G, B-H.1H, B-H.1, B-H.2, B-H.3, B-H.4, B-H.5, or B-H.6, or a sequence with at least 80%, 85%, 90%, 95%, %%, 97%, 98%, 99% or more identity thereto.

In some embodiments, the anti-TCRβVB 12 antibody molecule (e.g., anti-TCRβVB 12-3 or anti-TCRβVB 12-4 antibody molecule) comprises a VL of B-H.1A, B-H.1B, B-H.1C, B-H.1D, B-H.1E, B-H.1F, B-H.1G, B-H.1H, B-H.1, B-H.2, B-H.3, B-H.4, B-H.5, or B-H.6, or a sequence with at least 80%, 85%, 90%, 95%, 96%, 97%, 98%, 99% or more identity thereto.

In some embodiments, the anti-TCRβVB 12 antibody molecule (e.g., anti-TCRβVB 12-3 or anti-TCRβVB 12-4 antibody molecule) comprises a VH of B-H.1A, B-H.1B, B-H.1C, B-H.1D, B-H.1E, B-H.1F, B-H.1G, B-H.1H, B-H.1, B-H.2, B-H.3, B-H.4, B-H.5, or B-H.6, or a sequence with at least 80%, 85%, 90%, 95%, 96%, 97%, 98%, 99% or more identity thereto; and a VL of B-H.1A, B-H.1B, B-H.1C, B-H.1D, B-H.1E, B-H.1F, B-H.1G, B-H.1H, B-H.1, B-H.2, B-H.3, B-H.4, B-H.5, or B-H.6, or a sequence with at least 80%, 85%, 90%, 95%, 96%, 97%, 98%, 99% or more identity thereto.

### Anti-TCRβ V5 Antibodies

In one aspect, provided herein is an anti-TCRβV antibody molecule that binds to human TCRβ V5. In some embodiments, the TCRβ V5 subfamily comprises TCRβ V5-5*01, TCRβ 3 V5-6*01, TCRβ V5-4*01, TCRβ V5-8*01, TCRβ V5-1*01, or a variant thereof.

Exemplary anti-TCRβ V5 antibodies are provided in Table 10B. In some embodiments, the anti-TCRβ V5 is antibody C, e.g., humanized antibody C (antibody C-H), as provided in Table 10B. In some embodiments, the anti-TCRβV antibody comprises one or more (e.g., all three) of a LC CDR1, LC CDR2, and LC CDR3 provided in Table 10B, and/or one or more (e.g., all three) of a HC CDR1, HC CDR2, and HC CDR3 provided in Table 10B, or a sequence with at least 95% sequence identity thereto. In some embodiments, antibody C comprises a variable heavy chain (VH) and/or a variable light chain (VL) provided in Table 10B, or a sequence with at least 95% sequence identity thereto.

Exemplary anti-TCRβ V5 antibodies are provided in Table 11. In some embodiments, the anti-TCRβ V5 is antibody E, e.g., humanized antibody E (antibody E-H), as provided in Table 11. In some embodiments, the anti-TCRβV antibody comprises one or more (e.g., all three) of a LC CDR1, LC CDR2, and LC CDR3 provided in Table 11; and/or one or more (e.g., all three) of a HC CDR1. HC CDR2, and HC CDR3 provided in Table 11, or a sequence with at least 95% sequence identity thereto. In some embodiments, antibody E comprises a variable heavy chain (VH) and/or a variable light chain (VL) provided in Table 11, or a sequence with at least 95% sequence identity thereto.

In some embodiments, antibody E comprises a heavy chain comprising the amino acid sequence of SEQ ID NO: 3284 and/or a light chain comprising the amino acid sequence of SEQ ID NO: 3285, or a sequence with at least 95% sequence identity thereto.

In some embodiments, the anti-TCRβ V5 antibody molecule comprises a VH and/or a VL of an antibody described in Table 10B, or a sequence with at least 80%, 85%, 90%, 95%, 96%, 97%, 98%, 99% or more identity thereto.

In some embodiments, the anti-TCRβ V5 antibody molecule comprises a VH and a VL of an antibody described in Table 10B, or a sequence with at least 80%, 85%, 90%, 95%, 96%, 97%, 98%, 99% or more identity thereto.

In some embodiments, the anti-TCRβ V5 antibody molecule comprises a VH and/or a VL of an antibody described in Table 11, or a sequence with at least 80%, 85%, 90%, 95%, 96%, 97%, 98%, 99% or more identity thereto.

In some embodiments, the anti-TCRβ V5 antibody molecule comprises a VH and a VL of an antibody described in Table 11, or a sequence with at least 80%, 85%, 90%, 95%, 96%, 97%, 98%, 99% or more identity thereto.

### Anti-TCRβ V10 Antibodies

In one aspect, provided herein is an anti-TCRβV antibody molecule that binds to a human TCRβ V10 subfamily member. In some embodiments, TCRβ V10 subfamily is also known as TCRβ V12. In some embodiments, the TCRβ V10 subfamily comprises: TCRβ V10-1*01, TCRβ 3 V10-1*02, TCRβ V10-3*01 or TCRβ V10-2*01, or a variant thereof.

Exemplary anti-TCRβ V10 antibodies are provided in Table 12. In some embodiments, the anti-TCRβ V10 is antibody D, e.g., humanized antibody D (antibody D-H), as provided in Table 12. In some embodiments, antibody D comprises one or more (e.g., three) light chain CDRs and/or one or more (e.g., three) heavy chain CDRs provided in Table 12, or a sequence with at least 95% sequence identity thereto. In some embodiments, antibody D comprises a variable heavy chain (VH) and/or a variable light chain (VL) provided in Table 12, or a sequence with at least 95% sequence identity thereto.

In some embodiments, the anti-TCRβ V10 antibody molecule comprises a VH or a VL of an antibody described in Table 12, or a sequence with at least 80%, 85%, 90%, 95%, 96%, 97%, 98% 99% or more identity thereto.

In some embodiments, the anti-TCRβ V10 antibody molecule comprises a VH and a VL of an antibody described in Table 12, or a sequence with at least 80%, 85%, 90%, 95%, 96%, 97%, 98%, 99% or more identity thereto.

### Additional Anti-TCRVβ Antibodies

Additional exemplary anti-TCRβV antibodies are provided in Table 13. In some embodiments, the anti-TCRβV antibody is a humanized antibody, e.g., as provided in Table 13. In some embodiments, the anti-TCRβV antibody comprises one or more (e.g., all three) of a LC CDR1, LC CDR2, and LC CDR3 provided in Table 13; and/or one or more (e.g., all three) of a HC CDR1. HC CDR2, and HC CDR3 provided in Table 13, or a sequence with at least 95% sequence identity thereto. In some embodiments, the anti-TCRβV antibody comprises a variable heavy chain (VH) and/or a variable light chain (VL) provided in Table 13, or a sequence with at least 95% sequence identity thereto.

### Antibody-Like Frameworks or Scaffolds

A wide variety of antibody/immunoglobulin frameworks or scaffolds can be employed in the anti-TCRvb antibody molecules as described herein or multifunctional formats thereof so long as the resulting polypeptide includes at least one binding region which specifically binds to the target antigen. e.g., a TCRvb, a tumor antigen, among others. Such frameworks or scaffolds include the 5 main idiotypes of human immunoglobulins, or fragments thereof, and include immunoglobulins of other animal species, preferably having humanized aspects. Novel frameworks, scaffolds and fragments continue to be discovered and developed by those skilled in the art.

In some embodiments, the anti-TCRvb antibody molecules as described herein or multifunctional formats thereof include non-immunoglobulin based antibodies using non-immunoglobulin scaffolds onto which CDRs can be grafted. Any non-immunoglobulin frameworks and scaffolds may be employed, as long as they comprise a binding region specific for the target antigen (e.g., TCRvb or a tumor antigen). Exemplary non-immunoglobulin frameworks or scaffolds include, but are not limited to, fibronectin (Compound Therapeutics, Inc., Waltham, MA), ankyrin (Molecular Partners AG, Zurich, Switzerland), domain antibodies (Domantis, Ltd., Cambridge, MA, and Ablynx nv, Zwijnaarde, Belgium), lipocalin (Pieris Proteolab AG, Freising, Germany), small modular immuno-pharmaceuticals (Trubion Pharmaceuticals Inc., Seattle, WA), maxybodies (Avidia, Inc., Mountain View. CA), Protein A (Affibody AG, Sweden), and affilin (gamma-crystallin or ubiquitin) (Scil Proteins GmbH, Halle. Germany).

Fibronectin scaffolds are typically based on fibronectin type III domain (e.g., the tenth module of the fibronectin type III (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). Because of this structure, the non-immunoglobulin antibody mimics antigen binding properties that are similar in nature and affinity to those of antibodies. These scaffolds can be used in a loop randomization and shuffling strategy in vitro that is similar to the process of affinity maturation of antibodies in vivo. These fibronectin-based molecules can be used as scaffolds where the loop regions of the molecule can be replaced with CDRs of the invention using standard cloning techniques.

The ankyrin technology is based on using proteins with ankyrin derived repeat modules as scaffolds for bearing variable regions which can be used for binding to different targets. The ankyrin repeat module typically is a about 33 amino acid polypeptide consisting of two anti-parallel α-helices and a β-turn. Binding of the variable regions can be optimized by using ribosome display.

Avimers are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, U.S. Patent Application Publication Nos. 20040175756; 20050053973; 20050048512; and 20060008844.

Affibody affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa, compared to the molecular weight of antibodies, which is 150 kDa. In spite of its small size, the binding site of affibody molecules is similar to that of an antibody.

Anticalins are known commercially, e.g., Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acid residues, being just marginally bigger than a single immunoglobulin domain. The set of four loops, which makes up the binding pocket, shows pronounced structural plasticity and tolerates a variety of side chains. The binding site can thus be reshaped in a proprietary process in order to recognize prescribed target molecules of different shape with high affinity and specificity. One protein of lipocalin family, the bilin-binding protein (BBP) of Pieris Brassicae has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

Protein epitope mimetics (PEM) are medium-sized, cyclic, peptide-like molecules (MW 1-2 kDa) mimicking beta-hairpin secondary structures of proteins, the major secondary structure involved in protein-protein interactions.

Domain antibodies (dAbs) can be used in the anti-TCRvb antibody molecules as described herein or multifunctional formats thereof are small functional binding fragments of antibodies, corresponding to the variable regions of either the heavy or light chains of antibodies. Domain antibodies are well expressed in bacterial, yeast, and mammalian cell systems. Further details of domain antibodies and methods of production thereof are known in the art (see, for example, U.S. Pat. Nos. 6,291,158; 6,582,915; 6,593,081; 6,172,197; 6,696,245; European Patents 0368684 & 0616640; WO05/035572, WO04/101790, WO04/081026, WO04/058821, WO04/003019 and WO03/002609. Nanobodies are derived from the heavy chains of an antibody.

A nanobody typically comprises a single variable domain and two constant domains (CH2 and CH3) and retains antigen-binding capacity of the original antibody. Nanobodies can be prepared by methods known in the art (See e.g., U.S. Pat. Nos. 6,765,087, 6,838,254, WO 06/079372). Unibodies consist of one light chain and one heavy chain of an IgG4 antibody. Unibodies may be made by the removal of the hinge region of IgG4 antibodies. Further details of unibodies and methods of preparing them may be found in WO2007/059782.

### Anti-TCRβVB Antibody Effector Function and Fc Variants

In some embodiments, an anti-TCRVβ antibody as described herein comprises an Fc region, e.g., as described herein. In some embodiments, the Fc region is a wildtype Fc region, e.g., a wildtype human Fc region. In some embodiments, the Fc region comprises a variant, e.g., an Fc region comprising an addition, substitution, or deletion of at least one amino acid residue in the Fc region which results in, e.g., reduced or ablated affinity for at least one Fc receptor.

The Fc region of an antibody interacts with a number of receptors or ligands including Fc Receptors (e.g., FcγRI, FcγRIIA, FcγRIIIA), the complement protein CIq, and other molecules such as proteins A and G. These interactions are essential for a variety of effector functions and downstream signaling events including: antibody dependent cell-mediated cytotoxicity (ADCC). Antibody-dependent cellular phagocytosis (ADCP) and complement dependent cytotoxicity (CDC).

In some embodiments, an anti-TCRVβ antibody comprising a variant Fc region has reduced, e.g., ablated, affinity for an Fc receptor, e.g., an Fc receptor described herein. In some embodiments, the reduced affinity is compared to an otherwise similar antibody with a wildtype Fc region.

In some embodiments, an anti-TCRVβ antibody comprising a variant Fc region has one or more of the following properties: (1) reduced effector function (e.g., reduced ADCC. ADCP and/or CDC); (2) reduced binding to one or more Fc receptors; and/or (3) reduced binding to C1q complement. In some embodiments, the reduction in any one, or all of properties (1)-(3) is compared to an otherwise similar antibody with a wildtype Fc region.

In some embodiments, an anti-TCRVβ antibody comprising a variant Fc region has reduced affinity to a human Fc receptor, e.g., FcγR 1, FcγR II and/or FcγR III. In some embodiments, the anti-TCRVβ antibody comprising a variant Fc region comprises a human IgG1 region or a human IgG4 region.

In some embodiments, an anti-TCRβVB antibody comprising a variant Fc region activates and/or expands T cells, e.g., as described herein. In some embodiments, an anti-TCRβVs antibody comprising a variant Fc region has a cytokine profile described herein, e.g., a cytokine profile that differs from a cytokine profile of a T cell engager that binds to a receptor or molecule other than a TCRβV region (“a non-TCRβV-binding T cell engager”). In some embodiments, the non-TCRβV-binding T cell engager comprises an antibody that binds to a CD3 molecule (e.g., CD3 epsilon (CD3e) molecule); or a TCR alpha (TCRα) molecule.

Exemplary Fc region variants are provided in Table 14 and also disclosed in Saunders O, (2019) Frontiers in Immunology: vol 10, article1296, the entire contents of which is hereby incorporated by reference.

In some embodiments, an anti-TCRVβ antibody as described herein comprises any one or all, or any combination of Fc region variants disclosed in Table 14.

In some embodiments, an anti-TCRVβ antibody as described herein comprises any one or all, or any combination of Fc region variants, e.g., mutations, disclosed in Table 14. In some embodiments, an anti-TCRVβ antibody as described herein comprise an Asn297Ala (N297A) mutation. In some embodiments, an anti-TCRβVD antibody as described herein comprise a Leu234Ala/Leu235Ala (LALA) mutation.

### Multifunctional Molecules

As used herein, a “multifunctional” or a “multispecific” molecule refers to molecule, e.g., a polypeptide, that has two or more functionalities, e.g., two or more binding specificities. In some embodiments, the functionalities can include one or more immune cell engagers, one or more tumor binding molecules, one or more cytokine molecules, one or more stromal modifiers, and other moieties described herein. In some embodiments, the multispecific molecule is a multispecific antibody molecule. e.g., a bispecific antibody molecule. In some embodiments, the multispecific molecule includes an anti-TCRVb antibody molecule as described herein.

Described herein, in certain embodiments, is a multifunctional polypeptide molecule comprising a first polypeptide, a second polypeptide, a third polypeptide, a fourth polypeptide, and at least one cytokine polypeptide or a variant thereof, wherein the first polypeptide, the second polypeptide, the third polypeptide, and the fourth polypeptide are non-contiguous, wherein: (i) the first polypeptide comprising a first portion of a first T cell receptor variable beta (TCRβV)-binding moiety and a first dimerization module linked to the first portion of the first TCRβV-binding moiety; (ii) the second polypeptide comprising a second portion of the first TCRβV-binding moiety; (iii) the third polypeptide comprising a first portion of a second TCRβV-binding moiety and a second dimerization module linked to the first portion of the second TCRβV-binding moiety; and (iv) the fourth polypeptide comprising a second portion of the second TCRβV-binding moiety; and wherein the at least one cytokine polypeptide or the variant thereof is covalently linked to the first polypeptide, the second polypeptide, the third polypeptide, the fourth polypeptide, or a combination thereof.

Described herein, in certain embodiments, is a multifunctional polypeptide molecule comprising a first polypeptide, a second polypeptide, a third polypeptide, and at least one cytokine polypeptide or a variant thereof, wherein the first polypeptide, the second polypeptide, and the third polypeptide are non-contiguous, wherein: (i) the first polypeptide comprising a first portion of a first TCRβV-binding moiety and a first dimerization module linked to the first portion of the first TCRβV-binding moiety; (ii) the second polypeptide comprising a second portion of the first TCRβV-binding moiety; and (iii) the third polypeptide comprising a second dimerization module; and wherein the at least one cytokine polypeptide or the variant thereof is covalently linked to the first polypeptide, the second polypeptide, the third polypeptide, or a combination thereof.

Described herein, in certain embodiments, is a multifunctional polypeptide molecule comprising a first polypeptide, a second polypeptide, a third polypeptide, and at least one cytokine polypeptide or a variant thereof, wherein the first polypeptide, the second polypeptide, and the third polypeptide are non-contiguous, wherein: (i) the first polypeptide comprising a first portion of a first TCRβV-binding moiety and a first dimerization module linked to the first portion of the first TCRβV-binding moiety; (ii) the second polypeptide comprising a second portion of the first TCRβV-binding moiety; and (iii) the third polypeptide comprising a second dimerization module; wherein the at least one cytokine polypeptide or the variant thereof is covalently linked to the first polypeptide, the second polypeptide, the third polypeptide, or a combination thereof; and wherein the multifunctional polypeptide molecule does not comprise an additional TCRβV-binding moiety except the first TCRβV-binding moiety.

In some embodiments, the first portion of the first TCRβV-binding moiety comprises a first heavy chain variable domain (VH) and a first heavy chain constant domain 1 (CH1) linked to the first VH. In some embodiments, the first CH1 is linked to the C-terminus of the first VH. In some embodiments, the second portion of the first TCRβV-binding moiety comprises a first light chain variable domain (VL) and a first light chain constant domain (CL) linked to the first VL. In some embodiments, first CL is linked to the C-terminus of the first VL. In some embodiments, wherein the first dimerization module is linked to the first portion of the first TCRβV-binding moiety. In some embodiments, the first dimerization module is linked to the C-terminus of the first portion of the first TCRβV-binding moiety. In some embodiments, wherein the first portion of the second TCRβV-binding moiety comprises a second VH and a second CH1 linked to the second VH. In some embodiments, the second CH1 is linked to the C-terminus of the second VH. In some embodiments, the second portion of the second TCRβV-binding moiety comprises a second VL and a second CL linked to the second VL. In some embodiments, the second CL is linked to the C-terminus of the second VL. In some embodiments, the second dimerization module is linked to the first portion of the second TCRβV-binding moiety. In some embodiments, the second dimerization module is linked to the C-terminus of the first portion of the second TCRβV-binding moiety.

In some embodiments. (a) the N-terminus of the first polypeptide is linked to a first cytokine polypeptide or a variant thereof; the C-terminus of the first polypeptide is linked to a second cytokine poly peptide or a variant thereof; or a combination thereof; (b) the N-terminus of the second polypeptide is linked to a third cytokine polypeptide or a variant thereof; the C-terminus of the second polypeptide is linked to a fourth cytokine polypeptide or a variant thereof; or a combination thereof; (c) the N-terminus of the third polypeptide is linked to a fifth cytokine polypeptide or a variant thereof: the C-terminus of the third polypeptide is linked to a sixth cytokine polypeptide or a variant thereof; or a combination thereof: (d) the N-terminus of the fourth polypeptide is linked to a seventh cytokine polypeptide or a variant thereof: the C-terminus of the fourth polypeptide is linked to an eighth cytokine polypeptide or a variant thereof; or a combination thereof; or (e) a combination thereof.

In some embodiments. (a-1) the N-terminus of the first polypeptide is linked to the first cytokine polypeptide or the variant thereof; the C-terminus of the first polypeptide is linked to the second cytokine polypeptide or the variant thereof; or a combination thereof; and (a-2) the N-terminus of the second polypeptide is linked to the third cytokine polypeptide or the variant thereof; the C-terminus of the second polypeptide is linked to the fourth cytokine polypeptide or the variant thereof; or a combination thereof; (b-1) the N-terminus of the first polypeptide is linked to the first cytokine polypeptide or the variant thereof; the C-terminus of the first polypeptide is linked to the second cytokine polypeptide or the variant thereof; or a combination thereof; and (b-2) the N-terminus of the third polypeptide is linked to the fifth cytokine polypeptide or the variant thereof; the C-terminus of the third polypeptide is linked to the sixth cytokine polypeptide or the variant thereof; or a combination thereof; (c-1) the N-terminus of the first polypeptide is linked to the first cytokine polypeptide or the variant thereof: the C-terminus of the first polypeptide is linked to the second cytokine polypeptide or the variant thereof; or a combination thereof; and (c-2) the N-terminus of the fourth poly peptide is linked to the seventh cytokine poly peptide or the variant thereof; the C-terminus of the fourth polypeptide is linked to the eighth cytokine polypeptide or the variant thereof; or a combination thereof; (d-1) the N-terminus of the second polypeptide is linked to the third cytokine polypeptide or the variant thereof: the C-terminus of the second polypeptide is linked to the fourth cytokine polypeptide or the variant thereof; or a combination thereof; and (d-2) the N-terminus of the third polypeptide is linked to the fifth cytokine polypeptide or the variant thereof: the C-terminus of the third polypeptide is linked to the sixth cytokine polypeptide or the variant thereof; or a combination thereof. (e-1) the N-terminus of the second polypeptide is linked to the third cytokine polypeptide or the variant thereof; the C-terminus of the second polypeptide is linked to the fourth cytokine polypeptide or the variant thereof; or a combination thereof; and (e-2) the N-terminus of the fourth polypeptide is linked to the seventh cytokine polypeptide or the variant thereof: the C-terminus of the fourth polypeptide is linked to the eighth cytokine polypeptide or the variant thereof; or a combination thereof; or (f-1) the N-terminus of the third polypeptide is linked to the fifth cytokine polypeptide or the variant thereof, the C-terminus of the third polypeptide is linked to the sixth cytokine polypeptide or the variant thereof; or a combination thereof; and (f-2) the N-terminus of the fourth polypeptide is linked to the seventh cytokine polypeptide or the variant thereof; the C-terminus of the fourth polypeptide is linked to the eighth cytokine polypeptide or the variant thereof; or a combination thereof.

In some embodiments, (a-1) the N-terminus of the first polypeptide is linked to the first cytokine polypeptide or the variant thereof; the C-terminus of the first polypeptide is linked to the second cytokine polypeptide or the variant thereof; or a combination thereof; (a-2) the N-terminus of the second polypeptide is linked to the third cytokine polypeptide or the variant thereof; the C-terminus of the second polypeptide is linked to the fourth cytokine polypeptide or the variant thereof; or a combination thereof; and (a-3) the N-terminus of the third polypeptide is linked to the fifth cytokine polypeptide or the variant thereof; the C-terminus of the third polypeptide is linked to the sixth cytokine polypeptide or the variant thereof; or a combination thereof; (b-1) the N-terminus of the first polypeptide is linked to the first cytokine polypeptide or the variant thereof: the C-terminus of the first polypeptide is linked to the second cytokine polypeptide or the variant thereof, or a combination thereof; (b-2) the N-terminus of the second polypeptide is linked to the third cytokine polypeptide or the variant thereof: the C-terminus of the second polypeptide is linked to the fourth cytokine polypeptide or the variant thereof; or a combination thereof; and (b-3) the N-terminus of the fourth polypeptide is linked to the seventh cytokine polypeptide or the variant thereof: the C-terminus of the fourth polypeptide is linked to the eighth cytokine polypeptide or the variant thereof; or a combination thereof; or (c-1) the N-terminus of the second poly peptide is linked to the third cytokine polypeptide or the variant thereof: the C-terminus of the second polypeptide is linked to the fourth cytokine polypeptide or the variant thereof; or a combination thereof: (c-2) the N-terminus of the third polypeptide is linked to the fifth cytokine polypeptide or the variant thereof; the C-terminus of the third polypeptide is linked to the sixth cytokine polypeptide or the variant thereof; or a combination thereof; and (c-3) the N-terminus of the fourth polypeptide is linked to the seventh cytokine polypeptide or the variant thereof: the C-terminus of the fourth polypeptide is linked to the eighth cytokine poly peptide or the variant thereof; or a combination thereof.

In some embodiments. (1) the N-terminus of the first polypeptide is linked to the first cytokine polypeptide or the variant thereof; the C-terminus of the first polypeptide is linked to the second cytokine polypeptide or the variant thereof; or a combination thereof; (2) the N-terminus of the second polypeptide is linked to the third cytokine polypeptide or the variant thereof: the C-terminus of the second polypeptide is linked to the fourth cytokine polypeptide or the variant thereof; or a combination thereof: (3) the N-terminus of the third polypeptide is linked to the fifth cytokine polypeptide or the variant thereof: the C-terminus of the third polypeptide is linked to the sixth cytokine polypeptide or the variant thereof; or a combination thereof; and (4) the N-terminus of the fourth polypeptide is linked to the seventh cytokine polypeptide or the variant thereof: the C-terminus of the fourth polypeptide is linked to the eighth cytokine polypeptide or the variant thereof; or a combination thereof.

In some embodiments, the first cytokine polypeptide, the second cytokine polypeptide, or a combination thereof is within a single contiguous polypeptide chain of the first poly peptide, the third cytokine polypeptide, the fourth cytokine polypeptide, or a combination thereof is within a single contiguous polypeptide chain of the second polypeptide, the fifth cytokine polypeptide, the sixth cytokine polypeptide, or a combination thereof is within a single contiguous polypeptide chain of the third polypeptide, the seventh cytokine polypeptide, the eighth cytokine polypeptide, or a combination thereof is within a single contiguous poly peptide chain of the fourth poly peptide, or a combination thereof.

In some embodiments. (a) the N-terminus of the first polypeptide is linked to a first cytokine polypeptide or a variant thereof: the C-terminus of the first polypeptide is linked to a second cytokine polypeptide or a variant thereof; or a combination thereof; (b) the N-terminus of the second polypeptide is linked to a third cytokine polypeptide or a variant thereof; the C-terminus of the second polypeptide is linked to a fourth cytokine poly peptide or a variant thereof; or a combination thereof: (c) the N-terminus of the third polypeptide is linked to a fifth cytokine polypeptide or a variant thereof: the C-terminus of the third polypeptide is linked to a sixth cytokine polypeptide or a variant thereof; or a combination thereof; or (d) a combination thereof.

In some embodiments, (a-1) the N-terminus of the first polypeptide is linked to the first cytokine polypeptide or the variant thereof; the C-terminus of the first polypeptide is linked to the second cytokine polypeptide or the variant thereof; or a combination thereof; and (a-2) the N-terminus of the second polypeptide is linked to the third cytokine polypeptide or the variant thereof; the C-terminus of the second polypeptide is linked to the fourth cytokine polypeptide or the variant thereof; or a combination thereof; (b-1) the N-terminus of the first polypeptide is linked to the first cytokine polypeptide or the variant thereof; the C-terminus of the first polypeptide is linked to the second cytokine polypeptide or the variant thereof; or a combination thereof; and (b-2) the N-terminus of the third polypeptide is linked to the fifth cytokine polypeptide or the variant thereof; the C-terminus of the third polypeptide is linked to the sixth cytokine polypeptide or the variant thereof; or a combination thereof; or (c-1) the N-terminus of the second polypeptide is linked to the third cytokine polypeptide or the variant thereof: the C-terminus of the second polypeptide is linked to the fourth cytokine polypeptide or the variant thereof; or a combination thereof, and (c-2) the N-terminus of the third polypeptide is linked to the fifth cytokine polypeptide or the variant thereof; the C-terminus of the third polypeptide is linked to the sixth cytokine polypeptide or the variant thereof; or a combination thereof.

In some embodiments, (1) the N-terminus of the first polypeptide is linked to the first cytokine polypeptide or the variant thereof; the C-terminus of the first polypeptide is linked to the second cytokine polypeptide or the variant thereof; or a combination thereof; (2) the N-terminus of the second poly peptide is linked to the third cytokine polypeptide or the variant thereof; the C-terminus of the second polypeptide is linked to the fourth cytokine polypeptide or the variant thereof; or a combination thereof; and (3) the N-terminus of the third polypeptide is linked to the fifth cytokine polypeptide or the variant thereof: the C-terminus of the third polypeptide is linked to the sixth cytokine polypeptide or the variant thereof; or a combination thereof.

In some embodiments, the first cytokine polypeptide, the second cytokine polypeptide, or a combination thereof is within a single contiguous polypeptide chain of the first poly peptide, the third cytokine polypeptide, the fourth cytokine polypeptide, or a combination thereof is within a single contiguous polypeptide chain of the second polypeptide, the fifth cytokine polypeptide, the sixth cytokine polypeptide, or a combination thereof is within a single contiguous polypeptide chain of the third polypeptide, or a combination thereof.

In some embodiments, the multifunctional polypeptide molecule as described herein further comprises a linker between the first portion of the first TCRβV-binding moiety and the first dimerization module, a linker between the first portion of the second TCRβV-binding moiety and the second dimerization module, a linker between the first VH and the first CH1, a linker between the first VL and the first CL, a linker between the second VH and the second CH1, a linker between the second VL and the second CL, a linker between the at least one cytokine poly peptide or the variant thereof and the first polypeptide, a linker between the at least one cytokine polypeptide or the variant thereof and the second polypeptide, a linker between the at least one cytokine polypeptide or the variant thereof and the third polypeptide, a linker between the at least one cytokine polypeptide or the variant thereof and the fourth polypeptide, or a combination thereof.

In some embodiments, the multifunctional polypeptide molecule as described herein further comprises comprising a linker between the first portion of the first TCRβV-binding moiety and the first dimerization module, a linker between the first VH and the first CH1, a linker between the first VL and the first CL, a linker between the at least one cytokine polypeptide or the variant thereof and the first poly peptide, a linker between the at least one cytokine polypeptide or the variant thereof and the second polypeptide, a linker between the at least one cytokine polypeptide or the variant thereof and the third polypeptide, or a combination thereof. In some embodiments, linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

Described herein, in certain embodiments, is a multifunctional polypeptide molecule comprising a first polypeptide, a second polypeptide, a third polypeptide, a fourth polypeptide, a first cytokine polypeptide or a variant thereof, and a second cytokine polypeptide or a variant thereof, wherein the first polypeptide, the second polypeptide, the third polypeptide, and the fourth polypeptide are non-contiguous, wherein: (i) the first polypeptide comprising a first portion of a first TCRβV-binding moiety and a first dimerization module linked to the first portion of the first TCRβV-binding moiety; (ii) the second polypeptide comprising a second portion of the first TCRβV-binding moiety; (iii) the third polypeptide comprising a first portion of a second TCRβV-binding moiety and a second dimerization module linked to the first portion of the second TCRβV-binding moiety; and (iv) the fourth polypeptide comprising a second portion of the second TCRβV-binding moiety; and wherein the first cytokine polypeptide or the variant thereof is covalently linked to the C-terminus of the second polypeptide, and the second cytokine polypeptide or the variant thereof is covalently linked to the C-terminus of the fourth polypeptide.

Described herein, in certain embodiments, is a multifunctional poly peptide molecule comprising a first polypeptide, a second polypeptide, a third polypeptide, a fourth polypeptide, a cytokine polypeptide or a variant thereof, wherein the first polypeptide, the second polypeptide, the third polypeptide, and the fourth polypeptide are non-contiguous, wherein: (i) the first polypeptide comprising a first portion of a first TCRβV-binding moiety and a first dimerization module linked to the first portion of the first TCRβV-binding moiety; (ii) the second polypeptide comprising a second portion of the first TCRβV-binding moiety; (iii) the third polypeptide comprising a first portion of a second TCRβV-binding moiety and a second dimerization module linked to the first portion of the second TCRβV-binding moiety; and (iv) the fourth polypeptide comprising a second portion of the second TCRβV-binding moiety; and wherein the cytokine polypeptide or the variant thereof is covalently linked to the C-terminus of the second polypeptide or the C-terminus of the fourth polypeptide.

Described herein, in certain embodiments, is a multifunctional polypeptide molecule comprising a first polypeptide, a second polypeptide, a third polypeptide, a fourth polypeptide, a cytokine polypeptide or a variant thereof, wherein the first polypeptide, the second polypeptide, the third polypeptide, and the fourth polypeptide are non-contiguous, wherein: (i) the first polypeptide comprising a first portion of a first TCRβV-binding moiety and a first dimerization module linked to the first portion of the first TCRβV-binding moiety; (ii) the second polypeptide comprising a second portion of the first TCRβV-binding moiety; (iii) the third polypeptide comprising a first portion of a second TCRβV-binding moiety and a second dimerization module linked to the first portion of the second TCRβV-binding moiety; and (iv) the fourth polypeptide comprising a second portion of the second TCRβV-binding moiety; and wherein the cytokine poly peptide or the variant thereof is covalently linked to the C-terminus of the first polypeptide or the C-terminus of the third polypeptide.

Described herein, in certain embodiments, is a multifunctional polypeptide molecule comprising a first polypeptide, a second polypeptide, a third polypeptide, and a cytokine polypeptide or a variant thereof, wherein the first polypeptide, the second polypeptide, and the third polypeptide are non-contiguous, wherein: (i) the first polypeptide comprising a first portion of a first TCRβV-binding moiety and a first dimerization module linked to the first portion of the first TCRβV-binding moiety; (ii) the second polypeptide comprising a second portion of the first TCRβV-binding moiety; and (iii) the third polypeptide comprising a second dimerization module; wherein the at least one cytokine polypeptide or the variant thereof is covalently linked to the N terminus of the third polypeptide; and wherein the multifunctional polypeptide molecule does not comprise an additional TCRβV-binding moiety except the first TCRβV-binding moiety.

In some embodiments, the first portion of the first TCRβV-binding moiety comprises a first VH and a first CH1 linked to the first VH. In some embodiments, the first CH1 is linked to the C-terminus of the first VH.

In some embodiments, the second portion of the first TCRβV-binding moiety comprises a first VL and a first CL linked to the first VL. In some embodiments, first CL is linked to the C-terminus of the first VL.

In some embodiments, the first dimerization module is linked to the first portion of the first TCRβV-binding moiety. In some embodiments, the first dimerization module is linked to the C-terminus of the first portion of the first TCRβV-binding moiety. In some embodiments, the first portion of the second TCRβV-binding moiety comprises a second VH and a second CH1 linked to the second VH. In some embodiments, the second CH1 is linked to the C-terminus of the second VH. In some embodiments, the second portion of the second TCRβV-binding moiety comprises a second VL and a second CL linked to the second VL. In some embodiments, the second CL is linked to the C-terminus of the second VL. In some embodiments, the second dimerization module is linked to the first portion of the second TCRβV-binding moiety. In some embodiments, the second dimerization module is linked to the C-terminus of the first portion of the second TCRβV-binding moiety.

In some embodiments, the multifunctional polypeptide molecule as described herein further comprises a linker between the first portion of the first TCRβV-binding moiety and the first dimerization module, a linker between the first portion of the second TCRβV-binding moiety and the second dimerization module, a linker between the first VH and the first CH1, a linker between the first VL and the first CL, a linker between the second VH and the second CH1, a linker between the second VL and the second CL, a linker between the at least one cytokine polypeptide or the variant thereof and the first polypeptide, a linker between the at least one cytokine polypeptide or the variant thereof and the second poly peptide, a linker between the at least one cytokine polypeptide or the variant thereof and the third polypeptide, a linker between the at least one cytokine polypeptide or the variant thereof and the fourth polypeptide, or a combination thereof. In some embodiments, the multifunctional polypeptide molecule as described herein further comprises a linker between the first portion of the first TCRβV-binding moiety and the first dimerization module, a linker between the first VH and the first CH1, a linker between the first VL and the first CL, a linker between the at least one cytokine polypeptide or the variant thereof and the third polypeptide, or a combination thereof. In some embodiments, linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

In some embodiments, the first TCRβV-binding moiety, the second TCRβV-binding moiety, or a combination thereof comprises any one selected from the group consisting of a Fab, F(ab′)2, Fv, a single chain Fv (scFv), a single domain antibody, a diabody (dAb), a camelid antibody and a combination thereof. In some embodiments, the first TCRβV-binding moiety, the second TCRβV-binding moiety, or a combination thereof comprises a scFv or a Fab.

In some embodiments, the multifunctional polypeptide molecule does not comprise an additional antigen-binding moiety except the TCRβV-binding moiety. In some embodiments, the multifunctional polypeptide molecule further comprise an additional antigen-binding moiety that is not the TCRβV-binding moiety.

Described herein, in certain embodiments, is a multifunctional polypeptide molecule comprising a first polypeptide, a second polypeptide, and at least one cytokine polypeptide or a variant thereof, wherein the first polypeptide and the second polypeptide are non-contiguous, wherein: (i) the first polypeptide comprising a first TCRβV-binding moiety and a first dimerization module linked to the C-terminus of the first TCRβV-binding moiety, wherein the first TCRβV-binding moiety comprises a first VL and a first VH; and (ii) the second polypeptide comprising a second TCRβV-binding moiety and a second dimerization module linked to the C-terminus of the second TCRβV-binding moiety; wherein the at least one cytokine polypeptide or the variant thereof is covalently linked to the first polypeptide, the second polypeptide, or a combination thereof; wherein the first TCRβV-binding moiety, the second TCRβV-binding moiety, or a combination thereof comprises a scFv; and wherein the multifunctional polypeptide molecule does not comprise an additional antigen-binding moiety except the first TCRβV-binding moiety and the second TCRβV-binding moiety.

Described herein, in certain embodiments, is a multifunctional polypeptide molecule comprising a first polypeptide, a second polypeptide, and at least one cytokine polypeptide or a variant thereof, wherein the first polypeptide and the second polypeptide are non-contiguous, wherein: (i) the first polypeptide comprising a first TCRβV-binding moiety and a first dimerization module linked to the C-terminus of the first TCRβV-binding moiety, wherein the first TCRβV-binding moiety comprises a first VL and a first VH; and (ii) the second polypeptide comprising a second dimerization module; wherein the at least one cytokine polypeptide or the variant thereof is covalently linked to the first polypeptide, the second polypeptide, or a combination thereof; wherein the first TCRβV-binding moiety comprises a scFv; wherein the multifunctional polypeptide molecule does not comprise an additional antigen-binding moiety except the first TCRβV-binding moiety; and wherein the multifunctional polypeptide molecule does not comprise an additional TCRβV-binding moiety except the first TCRβV-binding moiety.

In some embodiments, (a) the N-terminus of the first polypeptide is linked to a first cytokine polypeptide or a variant thereof; the C-terminus of the first polypeptide is linked to a second cytokine polypeptide or a variant thereof; or a combination thereof; (b) the N-terminus of the second polypeptide is linked to a third cytokine polypeptide or a variant thereof: the C-terminus of the second polypeptide is linked to a fourth cytokine polypeptide or a variant thereof; or a combination thereof; or (e) a combination thereof.

In some embodiments, the first cytokine polypeptide, the second cytokine polypeptide, or a combination thereof is within a single contiguous polypeptide chain of the first polypeptide, the third cytokine polypeptide, the fourth cytokine polypeptide, or a combination thereof is within a single contiguous polypeptide chain of the second polypeptide, or a combination thereof.

In some embodiments, the multifunctional polypeptide molecule as described herein further comprises a linker between the first TCRβV-binding moiety and the first dimerization module, a linker between the second TCRβV-binding moiety and the second dimerization module, a linker between the at least one cytokine polypeptide or the variant thereof and the first polypeptide, a linker between the at least one cytokine polypeptide or the variant thereof and the second polypeptide, or a combination thereof.

In some embodiments, the multifunctional polypeptide molecule as described herein further comprises a linker between the first TCRβV-binding moiety and the first dimerization module, a linker between the at least one cytokine polypeptide or the variant thereof and the first polypeptide, a linker between the at least one cytokine polypeptide or the variant thereof and the second polypeptide, or a combination thereof. In some embodiments, the linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

In some embodiments, the multifunctional polypeptide molecule comprises at least two of the cytokine polypeptide. In some embodiments, the multifunctional polypeptide molecule comprises at least three of the cytokine polypeptide. In some embodiments, the multifunctional polypeptide molecule comprises at least four of the cytokine polypeptide. In some embodiments, the multifunctional polypeptide molecule comprises at least five of the cytokine polypeptide. In some embodiments, the multifunctional polypeptide molecule comprises at least six of the cytokine polypeptide. In some embodiments, the multifunctional polypeptide molecule comprises at least seven of the cytokine polypeptide. In some embodiments, the multifunctional polypeptide molecule comprises at least eight of the cytokine polypeptide. In some embodiments, the multifunctional polypeptide molecule comprises two of the cytokine polypeptide. In some embodiments, the multifunctional polypeptide molecule comprises three of the cytokine polypeptide. In some embodiments, the multifunctional polypeptide molecule comprises four of the cytokine polypeptide. In some embodiments, the multifunctional polypeptide molecule comprises five of the cytokine polypeptide. In some embodiments, the multifunctional polypeptide molecule comprises six of the cytokine polypeptide. In some embodiments, the multifunctional polypeptide molecule comprises seven of the cytokine polypeptide. In some embodiments, the multifunctional polypeptide molecule comprises eight of the cytokine polypeptide. In some embodiments, the multifunctional polypeptide molecule comprises two of the cytokine polypeptide, each of which is linked to the first polypeptide and the second polypeptide: the first polypeptide and the third polypeptide: the first polypeptide and the fourth polypeptide; the second and the third polypeptide; the second polypeptide and the fourth poly peptide; or the third polypeptide and the fourth polypeptide, respectively. In some embodiments, the multifunctional polypeptide molecule comprises three of the cytokine polypeptide, each of which is linked to the first poly peptide, the second polypeptide, and the third polypeptide: the first polypeptide, the second polypeptide, and the fourth polypeptide; the first polypeptide, the third polypeptide, and the fourth polypeptide; or the second polypeptide, the third polypeptide, and the fourth polypeptide, respectively. In some embodiments, the multifunctional polypeptide molecule comprises four of the cytokine polypeptide, each of which is linked to the first polypeptide, the second polypeptide, the third polypeptide, and the fourth polypeptide, respectively. In some embodiments, the cytokine polypeptide is not linked to the polypeptides that comprise the first TCRβV-binding moiety.

In some embodiments, the at least one cytokine polypeptide is selected from the group consisting of interleukin-2 (IL-2) or a fragment or a variant thereof, interleukin-7 (IL-7) or a fragment or a variant thereof, interleukin-12 (IL-12) or a fragment or a variant thereof, interleukin-15 (IL-15) or a fragment or a variant thereof, interleukin-18 (IL-18) or a fragment or a variant thereof, interleukin-21 (IL-21) or a fragment or a variant thereof, or interferon gamma or a fragment or a variant thereof, or a combination thereof.

In some embodiments, the at least one cytokine polypeptide comprises interleukin-2 (IL-2) or a fragment thereof. In some embodiments, the at least one cytokine poly peptide is interleukin-2 (IL-2) or a fragment thereof. In some embodiments, the at least one cytokine polypeptide comprises a sequence having at least 75%, 80%, 85%, 90%, 95%, 99%, 99.5%, 99.9%, or 100% sequence identity to the sequence of SEQ ID NO: 2191. In some embodiments, the at least one cytokine polypeptide comprises the sequence of SEQ ID NO: 2191. In some embodiments, the sequence of the at least one cytokine polypeptide is a sequence having at least 75%, 80%, 85%, 90%, 95%, 99%, 99.5%, 99.9%, or 100% sequence identity to the sequence of SEQ ID NO: 2191. In some embodiments, the sequence of the at least one cytokine polypeptide is the sequence of SEQ ID NO: 2191.

In some embodiments, the variant of the at least one cytokine polypeptide comprises an IL-2 variant comprising a mutation. In some embodiments, the mutation comprises an insertion mutation, a deletion mutation, or a substitution mutation. In some embodiments, the mutation comprises the substitution mutation. In some embodiments, the variant comprises an IL-2 variant comprising C125A mutation. In some embodiments, the variant of the at least one cytokine polypeptide is an IL-2 variant comprising a mutation. In some embodiments, the mutation is an insertion mutation, a deletion mutation, or a substitution mutation. In some embodiments, the mutation is the substitution mutation. In some embodiments, the variant is an 1L-2 variant comprising C125A mutation. In some embodiments, the variant comprises a sequence having at least 75%, 80%, 85%, 90%, 95%, 99%, 99.5%, 99.9%, or 100% sequence identity to the sequence of SEQ ID NO: 2270. In some embodiments, the variant comprises the sequence of SEQ ID NO: 2270. In some embodiments, the sequence of the variant is a sequence having at least 75%, 80%, 85%, 90%, 95%, 99%, 99.5%, 99.9%, or 100% sequence identity to the sequence of SEQ ID NO: 2270. In some embodiments, the sequence of the variant is the sequence of SEQ ID NO: 2270.

In some embodiments, the first dimerization module comprises a first immunoglobulin constant regions (Fc regions) and the second dimerization module comprises a second Fc region. In some embodiments, the first dimerization module is a first immunoglobulin constant regions (Fc regions) and the second dimerization module is a second Fc region.

In some embodiments, the first Fc region, the second Fc region, or a combination thereof is selected from an IgG1 Fc region or a fragment thereof, an IgG2 Fc region or a fragment thereof, an IgG3 Fc region or a fragment thereof, an IgGA1 Fc region or a fragment thereof, an IgGA2 Fc region or a fragment thereof, an IgG4 Fc region or a fragment thereof, an IgJ Fc region or a fragment thereof, an IgM Fc region or a fragment thereof, an IgD Fc region or a fragment thereof, and an IgE Fc region or a fragment thereof.

In some embodiments, the first Fc region, the second Fc region, or a combination thereof is selected from a human IgG1 Fc region or a fragment thereof, a human IgG2 Fc region or a fragment thereof, and a human IgG4 Fc region or a fragment thereof.

In some embodiments, the first Fc region, the second Fc region, or a combination thereof comprises an Fc interface with one or more of: a paired cavity-protuberance, an electrostatic interaction, or a strand-exchange, wherein the dimerization of the first Fc region and the second Fc region is enhanced as indicated by a greater ratio of heteromultimer:homomultimer forms relative to a dimerization of Fc regions with a non-engineered interface. In some embodiments, the dimerization of the first Fc region and the second Fc region is enhanced at least by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface. In some embodiments, the dimerization of the first Fc region and the second Fc region is enhanced at most by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface. In some embodiments, the dimerization of the first Fc region and the second Fc region is enhanced by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 900) fold, or 10000 fold relative to a dimerization of Fc regions with a non-engineered interface.

In some embodiments, the first Fc region, the second Fc region, or a combination thereof comprises an amino acid substitution listed in Table 14.

In some embodiments, the first Fc region, the second Fc region, or a combination thereof comprises an Asn297Ala (N297A) mutation or a Leu234Ala/Leu235Ala (LALA) mutation.

In some embodiments, the first Fc region, the second Fc region, or a combination thereof comprises a sequence having at least 75%, 80%, 85%, 90%, 95%, 99%, 99.5%, 99.9%, or 100% sequence identity to the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649. In some embodiments, the first Fc region, the second Fc region, or a combination thereof comprises the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649.

In some embodiments, the sequence of the first Fc region, the second Fc region, or a combination thereof is a sequence having at least 75%, 80%, 85%, 90%, 95%, 99%, 99.5%, 99.9%, or 100% sequence identity to the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649. In some embodiments, the sequence of the first Fc region, the second Fc region, or a combination thereof is the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649.

In some embodiments, the first TCRβV-binding moiety, the second TCRβV-binding moiety, or a combination thereof binds to one or more of a TCRβV subfamily selected from the group consisting of: (i) TCRβ V2 subfamily comprising TCRβ V2*01; (ii) TCRβ V3 subfamily comprising TCRβ V3-1*01; (iii) TCRβ V4 subfamily comprising one or more selected from TCRβ V4-1, TCRβ V4-2, and TCRβ V4-3; (iv) TCRβ V5 subfamily comprising one or more selected from TCRβ V5-6*01, TCRβ V5-4*01, TCRβ V5-1*01, and TCRβ V5-8*01; (v) the TCRβ V6 subfamily comprising one or more selected from TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01, and TCRβ V6-1*01; (vi) TCRβ V9 subfamily; (vii) TCRβ V10 subfamily comprising one or more selected from TCRβ V10-1*01, TCRβ V10-1*02, TCRβ V10-3*01, and TCRβ V10-2*01: (viii) TCRβ V11 subfamily comprising TCRβ V11-2; (ix) TCRβ V12 subfamily comprising one or more selected from TCRβ V12-4*01, TCRβ V12-3*01, and TCRβ V12-5*01; (x) TCRβ V13 subfamily comprising TCRβ V13*01: (xi) TCRβ V16 subfamily comprising TCRβ V16*01; (xii) TCRβ V19 subfamily comprising one or more selected from TCRβ V19*01 and TCRβ V19*02: (xiii) TCRβ V21 subfamily. (xiv) TCRβ V23 subfamily. (xv) TCRβ V27 subfamily; and (xvi) TCRβ V28 subfamily.

In some embodiments, the first TCRβV-binding moiety and the second TCRβV-binding moiety are same. In some embodiments, the first TCRβV-binding moiety and the second TCRβV-binding moiety are different.

In some embodiments, the first TCRβV-binding moiety and the second TCRβV-binding moiety binds; (i) one or more of a TCRβ V6 subfamily member and one or more of a TCRβ V10 subfamily member, respectively; (ii) one or more of a TCRβ V6 subfamily member and one or more of a TCRβ V5 subfamily member, respectively; (iii) one or more of a TCRβ V6 subfamily member and one or more of a TCRβ V12 subfamily member, respectively; (iv) one or more of a TCRβ V10 subfamily member and one or more of a TCRβ V5 subfamily member, respectively; (v) one or more of a TCRβ V10 subfamily member and one or more of a TCRβ V12 subfamily member, respectively; or (vi) one or more of a TCRβ V5 subfamily member and one or more of a TCRβ V12 subfamily member, respectively.

In some embodiments, the first TCRβV-binding moiety, the second TCRβV-binding moiety, or a combination thereof comprises: (i) a HC CDR1, a HC CDR2 and a HC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 1: (ii) a LC CDR 1, a LC CDR2, and a LC CDR3 of an amino acid sequence having at least 75%, 80%, 85%, 90%, 95%, 99%, 99.5%, 99.9%, or 100% sequence identity to any one of the CDR 1, CDR2, and CDR3 the sequences listed in Table 1; or (iii) a combination thereof. In some embodiments, the first TCRβV-binding moiety, the second TCRβV-binding moiety, or a combination thereof comprises: (i) a HC CDR1, a HC CDR2 and a HC CDR3 having any one of the CDR 1, CDR2, and CDR3 sequences listed in Table 1: (ii) a LC CDR 1, a LC CDR2, and a LC CDR3 having any one of the CDR1, CDR2, and CDR3 the sequences listed in Table 1; or (iii) a combination thereof.

In some embodiments, the first TCRβV-binding moiety, the second TCRβV-binding moiety, or a combination thereof comprises: (i) a HC CDR1, a HC CDR2 and a HC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 1, respectively; (ii) a LC CDR1, a LC CDR2, and a LC CDR3 of an amino acid sequence having at least 75%, 80%, 85%, 90%, 95%, 99%, 99.5%, 99.9%, or 100% sequence identity to any one of the CDR1, CDR2, and CDR3 the sequences listed in Table 1, respectively; or (iii) a combination thereof. In some embodiments, the first TCRβV-binding moiety, the second TCRβV-binding moiety, or a combination thereof comprises: (i) a HC CDR1, a HC CDR2 and a HC CDR3 having any one of the CDR1, CDR2, and CDR3 sequences listed in Table 1, respectively; (ii) a LC CDR1, a LC CDR2, and a LC CDR3 having any one of the CDR1, CDR2, and CDR3 the sequences listed in Table 1, respectively; or (iii) a combination thereof.

In some embodiments, the first TCRβV-binding moiety, the second TCRβV-binding moiety, or a combination thereof comprises: (i) a VH comprising a framework region (FR) comprising a framework 1 (FR1), a framework region 2 (FR2), a framework region 3 (FR3), and a framework region 4 (FR4) that have at least 75%, 80%, 85%, 90%, 95%, 99%, 99.5%, 99.9%, or 100% sequence identity with a non-murine germline FR 1, a non-murine germline FR2, a non-murine germline FR3, and a non-murine germline FR4; (ii) a VL comprising a FR comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4; or (iii) a combination thereof. In some embodiments, the first TCRβV-binding moiety, the second TCRβV-binding moiety, or a combination thereof comprises: (i) a VH comprising a FR comprising a FR 1, a FR2, a FR3, and a FR4 having the sequences of a non-murine germline FR1, a non-murine germline FR2, a non-murine germline FR3, and a non-murine germline FR4; (ii) a VL comprising a FR comprising a FR1, a FR2, a FR3, and a FR4 having the sequences of a non-murine germline FR1, a non-murine germline FR2, a non-murine germline FR3, and a non-murine germline FR4; or (iii) a combination thereof.

In some embodiments, the first TCRβV-binding moiety, the second TCRβV-binding moiety, or a combination thereof comprises: (i) a VH comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4, respectively; (ii) a VL comprising a FR comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4, respectively; or (iii) a combination thereof. In some embodiments, the first TCRβV-binding moiety, the second TCRβV-binding moiety, or a combination thereof comprises: (i) a VH comprising a FR comprising a FR1, a FR2, a FR3, and a FR4 having the sequences of a non-murine germline FR1, a non-murine germline FR2, a non-murine germline FR3, and a non-murine germline FR4, respectively; (ii) a VL comprising a FR comprising a FR1, a FR2, a FR3, and a FR4 having the sequences of a non-murine germline FR1, a non-murine germline FR2, a non-murine germline FR3, and a non-murine germline FR4, respectively; or (iii) a combination thereof.

In some embodiments, the VH comprises the FR3 comprising (i) a Threonine at position 73 according to Kabat numbering; (ii) a Glycine a position 94 according to Kabat numbering; or (iii) a combination thereof. In some embodiments, the VL comprises the FR1 comprising a Phenyalanine at position 10 according to Kabat numbering. In some embodiments, the VL comprises the FR2 comprising (i) a Histidine at position 36 according to Kabat numbering; (ii) an Alanine at position 46 according to Kabat numbering; or (iii) a combination thereof. In some embodiments, the VL comprises the FR3 comprising a Phenyalanine at position 87 according to Kabat numbering.

In some embodiments, the first TCRβV-binding moiety, the second TCRβV-binding moiety, or a combination thereof comprises: (i) a HC CDR1, a HC CDR2 and a HC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 2; (ii) a LC CDR1, a LC CDR2, and a LC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 2; or (iii) a combination thereof. In some embodiments, the first TCRβV-binding moiety, the second TCRβV-binding moiety, or a combination thereof comprises: (i) a HC CDR1, a HC CDR2 and a HC CDR3 having any one of the CDR1, CDR2, and CDR3 sequences listed in Table 2; (ii) a LC CDR1, a LC CDR2, and a LC CDR3 having any one of the CDR1, CDR2, and CDR3 sequences listed in Table 2; or (iii) a combination thereof.

In some embodiments, the first TCRβV-binding moiety, the second TCRβV-binding moiety, or a combination thereof comprises: (i) a HC CDR1, a HC CDR2 and a HC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 2, respectively; (ii) a LC CDR1, a LC CDR2, and a LC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 2, respectively; or (iii) a combination thereof. In some embodiments, the first TCRβV-binding moiety, the second TCRβV-binding moiety, or a combination thereof comprises: (i) a HC CDR1, a HC CDR2 and a HC CDR3 having any one of the CDR1, CDR2, and CDR3 sequences listed in Table 2, respectively; (ii) a LC CDR1, a LC CDR2, and a LC CDR3 having any one of the CDR1, CDR2, and CDR3 sequences listed in Table 2, respectively; or (iii) a combination thereof.

In some embodiments, the first TCRβV-binding moiety, the second TCRβV-binding moiety, or a combination thereof comprises: (i) a VH comprising a FR comprising a FR1, a FR2, a FR3, and a FR4 that have at least 75%, 80%, 85%, 90%, 95%, 99%, 99.5%, 99.9%, or 1000% sequence identity with a FR1, a FR2, a FR3, and a FR4 of a humanized B-H LC of Table 2; (ii) a VL comprising a FR comprising a FR1, a FR2, a FR3, and a FR4 that have at least 75%, 80%, 85%, 90%, 95%, 99%, 99.5%, 99.9%, or 100% sequence identity with a FR1, a FR2, a FR3, and a FR4 of a humanized B-H LC of Table 2; or (iii) a combination thereof. In some embodiments, the first TCRβV-binding moiety, the second TCRβV-binding moiety, or a combination thereof comprises: (i) a VH comprising a FR comprising a FR 1, a FR2, a FR3, and a FR4 that have the sequence of a FR1, a FR2, a FR3, and a FR4 of a humanized B-H LC of Table 2; (ii) a VL comprising a FR comprising a FR1, a FR2, a FR3, and a FR4 that have the sequence of a FR1, a FR2, a FR3, and a FR4 of a humanized B-H LC of Table 2; or (iii) a combination thereof.

In some embodiments, the first TCRβV-binding moiety, the second TCRβV-binding moiety, or a combination thereof comprises: (i) a VH comprising a FR comprising a FR1, a FR2, a FR3, and a FR4 that have at least 75%, 80%, 85%, 90%, 95%, 99%, 99.5%, 99.9%, or 100% sequence identity with a FR1, a FR2, a FR3, and a FR4 of a humanized B-H LC of Table 2, respectively; (ii) a VL comprising a FR comprising a FR1, a FR2, a FR3, and a FR4 that have at least 75%, 80%, 85%, 90%, 95%, 99%, 99.5%, 99.9%, or 100% sequence identity with a FR1, a FR2, a FR3, and a FR4 of a humanized B-H LC of Table 2, respectively; or (iii) a combination thereof. In some embodiments, the first TCRβV-binding moiety, the second TCRβV-binding moiety, or a combination thereof comprises: (i) a VH comprising a FR comprising a FR1, a FR2, a FR3, and a FR4 that have the sequence of a FR1, a FR2, a FR3, and a FR4 of a humanized B-H LC of Table 2, respectively; (ii) a VL comprising a FR comprising a FR1, a FR2, a FR3, and a FR4 that have the sequence of a FR1, a FR2, a FR3, and a FR4 of a humanized B-H LC of Table 2, respectively; or (iii) a combination thereof.

In some embodiments, the first TCRβV-binding moiety, the second TCRβV-binding moiety, or a combination thereof comprises: (i) a VH comprising a sequence having at least 75%, 80%, 85%, 90%, 95%, 99%, 99.5%, 99.9%, or 100% sequence identity to the VH sequence of a humanized Antibody B-H listed in Table 2: (ii) a VL comprising a sequence having at least at least 75%, 80%, 85%, 90%, 95%, 99%, 99.5%, 99.9%, or 100% sequence identity to the VL sequence of a humanized Antibody B-H listed in Table 2; or (iii) a combination thereof. In some embodiments, the first TCRβV-binding moiety, the second TCRβV-binding moiety, or a combination thereof comprises: (i) a VH comprising the VH sequence of a humanized Antibody B-H listed in Table 2; (ii) a VL comprising the VL sequence of a humanized Antibody B-H listed in Table 2; or (iii) a combination thereof. In some embodiments, the first TCRβV-binding moiety, the second TCRβV-binding moiety, or a combination thereof comprises: (i) the VH of a humanized Antibody B-H listed in Table 2. (ii) the VL sequence of a humanized Antibody B-H listed in Table 2; or (iii) a combination thereof.

In some embodiments, the first polypeptide, the second polypeptide, the third polypeptide, the fourth polypeptide, or a combination thereof comprises a heavy chain constant region having a sequence having at least 75%, 80%, 85%, 90%, 95%, 99%, 99.5%, 99.9%, or 100% sequence identity to any one of the sequences listed in Table 3 or a combination thereof. In some embodiments, the first polypeptide, the second polypeptide, the third polypeptide, the fourth polypeptide, or a combination thereof comprises a heavy chain constant region having any one of the sequences listed in Table 3 or a combination thereof. In some embodiments, the first polypeptide, the second polypeptide, the third polypeptide, the fourth poly peptide, or a combination thereof comprises a heavy chain constant region of which sequence is a sequence having at least 75%, 80%, 85%, 90%, 95%, 99%, 99.5%, 99.9%, or 100% sequence identity to any one of the sequences listed in Table 3 or a combination thereof. In some embodiments, the first polypeptide, the second polypeptide, the third polypeptide, the fourth polypeptide, or a combination thereof comprises a heavy chain constant region having any one of the heavy chain constant region sequences listed in Table 3 or a combination thereof. In some embodiments, the first poly peptide, the second polypeptide, the third polypeptide, the fourth polypeptide, or a combination thereof comprises a heavy chain constant region of an IgM or a fragment thereof. In some embodiments, the heavy chain constant region of the IgM comprises a sequence having at least 75%, 80%, 85%, 90%, 95%, 99%, 99.5%, 99.9%, or 100% sequence identity to the sequence of SEQ ID NO: 73. In some embodiments, the heavy chain constant region of the IgM comprises the sequence of SEQ ID NO: 73. In some embodiments, the sequence of the heavy chain constant region of the IgM is the sequence of SEQ ID NO: 73.

In some embodiments, the first polypeptide, the second polypeptide, the third polypeptide, the fourth polypeptide, or a combination thereof comprises a heavy chain constant region of an IgJ or a fragment thereof. In some embodiments, the heavy chain constant region of the IgJ comprises a sequence having at least 75%, 80%, 85%, 90%, 95%, 99%, 99.5%, 99.9%, or 100% sequence identity to the sequence of SEQ ID NO: 76. In some embodiments, the heavy chain constant region of the IgJ comprises the sequence of SEQ ID NO: 76. In some embodiments, the sequence of the heavy chain constant region of the IgJ is the sequence of SEQ ID NO: 76.

In some embodiments, the first polypeptide, the second polypeptide, the third polypeptide, the fourth polypeptide, or a combination thereof comprises a heavy chain constant region of an IgGA1 or a fragment thereof. In some embodiments, the heavy chain constant region of the IgGA1 comprises a sequence having at least 75%, 80%, 85%, 90%, 95%, 99%, 99.5%, 99.9%, or 100% sequence identity to the sequence of SEQ ID NO: 74. In some embodiments, the heavy chain constant region of the IgGA1 comprises the sequence of SEQ ID NO: 74. In some embodiments, the sequence of the heavy chain constant region of the IgGA1 is the sequence of SEQ ID NO: 74.

In some embodiments, the first polypeptide, the second polypeptide, the third polypeptide, the fourth polypeptide, or a combination thereof comprises a heavy chain constant region of an IgGA2 or a fragment thereof. In some embodiments, the heavy chain constant region of the IgGA2 comprises a sequence having at least 75%, 80%, 85%, 90%, 95%, 99%, 99.5%, 99.9%, or 100% sequence identity to the sequence of SEQ ID NO: 75. In some embodiments, the heavy chain constant region of the IgGA2 comprises the sequence of SEQ ID NO: 75. In some embodiments, the sequence of the heavy chain constant region of the IgGA2 is the sequence of SEQ ID NO: 75.

In some embodiments, the first polypeptide, the second polypeptide, the third polypeptide, the fourth polypeptide, or a combination thereof comprises a heavy chain constant region of an IgG1 or a fragment thereof. In some embodiments, the heavy chain constant region of the IgG1 comprises a sequence having at least 75%, 80%, 85%, 90%, 95%, 99%, 99.5%, 99.9%, or 100% sequence identity to the sequence of SEQ ID NO: 41. In some embodiments, the heavy chain constant region of the IgG1 comprises the sequence of SEQ ID NO: 41. In some embodiments, the sequence of the heavy chain constant region of the IgG1 is the sequence of SEQ ID NO: 41. In some embodiments, the heavy chain constant region of the IgG1 comprises a sequence having at least 75%, 80%, 85%, 90%, 95%, 99%, 99.5%, 99.9%, or 100% sequence identity to the sequence of SEQ ID NO: 3645. In some embodiments, the heavy chain constant region of the IgG1 comprises the sequence of SEQ ID NO: 3645. In some embodiments, the sequence of the heavy chain constant region of the IgG1 is the sequence of SEQ ID NO: 3645.

In some embodiments, the first polypeptide, the second polypeptide, the third polypeptide, the fourth polypeptide, or a combination thereof comprises a light chain constant region having a sequence having at least 75%, 80%, 85%, 90%, 95%, 99%, 99.5%, 99.9%, or 100% sequence identity to any one of the sequences listed in Table 3 or a combination thereof. In some embodiments, the first polypeptide, the second polypeptide, the third polypeptide, the fourth polypeptide, or a combination thereof comprises a light chain constant region having any one of the sequences listed in Table 3 or a combination thereof. In some embodiments, the first polypeptide, the second polypeptide, the third polypeptide, the fourth polypeptide, or a combination thereof comprises a light chain constant region having any one of the light chain constant region sequences listed in Table 3 or a combination thereof.

In some embodiments, the first polypeptide, the second polypeptide, the third polypeptide, the fourth poly peptide, or a combination thereof comprises a light chain constant region of a kappa chain or a fragment thereof. In some embodiments, the light chain constant region of a kappa chain comprises a light chain constant region sequence listed in Table 3.

In some embodiments, the light chain constant region of a kappa chain comprises a sequence having at least 75%, 80%, 85%, 90%, 95%, 99%, 99.5%, 99.9%, or 100% sequence identity to the sequence of SEQ ID NO: 39 or SEQ ID NO: 3644. In some embodiments, the light chain constant region of a kappa chain comprises the sequence of SEQ ID NO: 39 or SEQ ID NO: 3644. In some embodiments, the sequence of the light chain constant region of a kappa chain is a sequence having at least 75%, 80%, 85%, 90%, 95%, 99%, 99.5%, 99.9%, or 100% sequence identity to the sequence of SEQ ID NO: 39 or SEQ ID NO: 3644. In some embodiments, the sequence of the light chain constant region of a kappa chain is the sequence of SEQ ID NO: 39 or SEQ ID NO: 3644.

In some embodiments, the first TCRβV-binding moiety, the second TCRβV-binding moiety, or a combination thereof comprises: (i) a HC CDR1, a HC CDR2 and a HC CDR3 comprising amino acid sequences having at least 75%, 80%, 85%, 90%, 95%, 99%, 99.5%, 99.9%, or 100% sequence identity to CDR1, CDR2, and CDR3 sequences of a VH disclosed in Tables 1, 2, 10, 11, 12 or 13. (ii) a LC CDR1, a LC CDR2, and a LC CDR3 comprising an amino acid sequence having at least 75%, 80%, 85%, 90%, 95%, 99%, 99.5%, 99.9%, or 100% sequence identity to CDR1, CDR2, and CDR3 sequences of a VL disclosed in Tables 1, 2, 10, 11, 12 or 13; or (iii) a combination thereof. In some embodiments, the first TCRβV-binding moiety, the second TCRβV-binding moiety, or a combination thereof comprises: (i) a HC CDR1, a HC CDR2 and a HC CDR3 comprising the CDR1, CDR2, and CDR3 sequences of a VH disclosed in Tables 1, 2, 10, 11, 12 or 13; (ii) a LC CDR1, a LC CDR2, and a LC CDR3 comprising the CDR1, CDR2, and CDR3 sequences of a VL disclosed in Tables 1, 2, 10, 11, 12 or 13; or (iii) a combination thereof. In some embodiments, the first TCRβV-binding moiety, the second TCRβV-binding moiety, or a combination thereof comprises: (i) a HC CDR1, a HC CDR2 and a HC CDR3 comprising amino acid sequences having at least 75%, 80%, 85%, 90%, 95%, 99%, 99.5%, 99.9%, or 100% sequence identity to CDR1, CDR2, and CDR3 sequences of a VH disclosed in Tables 1, 2, 10, 11, 12 or 13, respectively; (ii) a LC CDR1, a LC CDR2, and a LC CDR3 comprising an amino acid sequence having at least 75%, 80%, 85%, 90%, 95%, 99%, 99.5%, 99.9%, or 100% sequence identity to CDR1, CDR2, and CDR3 sequences of a VL disclosed in Tables 1, 2, 10, 11, 12 or 13, respectively; or (iii) a combination thereof. In some embodiments, the first TCRβV-binding moiety, the second TCRβV-binding moiety, or a combination thereof comprises: (i) a HC CDR1, a HC CDR2 and a HC CDR3 comprising the CDR1, CDR2, and CDR3 sequences of a VH disclosed in Tables 1, 2, 10, 11, 12 or 13, respectively; (ii) a LC CDR1, a LC CDR2, and a LC CDR3 comprising the CDR1, CDR2, and CDR3 sequences of a VL disclosed in Tables 1, 2, 10, 11, 12 or 13, respectively; or (iii) a combination thereof. In some embodiments, the first TCRβV-binding moiety, the second TCRβV-binding moiety, or a combination thereof comprises: (i) a HC CDR1, a HC CDR2 and a HC CDR3 of a VH disclosed in Tables 1, 2, 10, 11, 12 or 13; (ii) a LC CDR1, a LC CDR2, and a LC CDR3 of a VL disclosed in Tables 1, 2, 10, 11, 12 or 13; or (iii) a combination thereof.

In some embodiments, the first TCRβV-binding moiety, the second TCRβV-binding moiety, or a combination thereof comprises a light chain comprising a FR1 comprising: (i) an Aspartic Acid at position 1 according to Kabat numbering; (ii) an Asparagine at position 2 according to Kabat numbering. (iii) a Leucine at position 4 according to Kabat numbering; or (iv) a combination thereof.

In some embodiments, the first TCRβV-binding moiety, the second TCRβV-binding moiety, or a combination thereof comprises a light chain comprising a FR3 comprising: (i) a Glycine at position 66 according to Kabat numbering; (ii) an Asparagine at position 69 according to Kabat numbering; (iii) a Tyrosine at position 71 according to Kabat numbering; or (iv) a combination thereof.

In some embodiments, the first TCRβV-binding moiety, the second TCRβV-binding moiety, or a combination thereof binds to an outward facing region on a TCRβV protein. In some embodiments, the outward facing region on the TCRβV protein comprises a structurally conserved region of TCRβV having a similar structure across one or more TCRβV subfamilies.

### Cytokine Molecules

In some embodiments, the multifunctional molecule includes a cytokine molecule. As used herein, a “cytokine molecule” refers to full length, a fragment or a variant of a cytokine; a cytokine further comprising a receptor domain, e.g., a cytokine receptor dimerizing domain; or an agonist of a cytokine receptor, e.g., an antibody molecule (e.g., an agonistic antibody) to a cytokine receptor, that elicits at least one activity of a naturally-occurring cytokine. In some embodiments the cytokine molecule is chosen from interleukin-2 (IL-2), interleukin-7 (IL-7), interleukin-12 (IL-12), interleukin-10 (IL-10), interleukin-15 (IL-15), interleukin-18 (IL-18), interleukin-21 (IL-21), or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain. In other embodiments, the cytokine molecule is an agonist of a cytokine receptor, e.g., an antibody molecule (e.g., an agonistic antibody) to a cytokine receptor chosen from an IL-15Ra or IL-21R.

Cytokines are generally polypeptides that influence cellular activity, for example, through signal transduction pathways. Accordingly, a cytokine of the multispecific or multifunctional polypeptide is useful and can be associated with receptor-mediated signaling that transmits a signal from outside the cell membrane to modulate a response within the cell. Cytokines are proteinaceous signaling compounds that are mediators of the immune response. They control many different cellular functions including proliferation, differentiation and cell survival/apoptosis; cytokines are also involved in several pathophysiological processes including viral infections and autoimmune diseases. Cytokines are synthesized under various stimuli by a variety of cells of both the innate (monocytes, macrophages, dendritic cells) and adaptive (T- and B-cells) immune systems. Cytokines can be classified into two groups: pro- and anti-inflammatory. Pro-inflammatory cytokines, including IFNγ, IL-1, IL-6 and TNF-alpha, are predominantly derived from the innate immune cells and Th1 cells. Anti-inflammatory cytokines, including IL-10, IL-4, IL-13 and IL-5, are synthesized from Th2 immune cells.

Provided herein are, inter alia, multispecific (e.g., bi-, tri-, quad-specific) or multifunctional molecules, that include, e.g., are engineered to contain, one or more cytokine molecules, e.g., immunomodulatory (e.g., proinflammatory) cytokines and variants, e.g., functional variants, thereof. Accordingly, in some embodiments, the cytokine molecule is an interleukin or a variant, e.g., a functional variant thereof. In some embodiments the interleukin is a proinflammatory interleukin. In some embodiments the interleukin is chosen from interleukin-2 (IL-2), interleukin-12 (IL-12), interleukin-15 (IL-15), interleukin-18 (IL-18), interleukin-21 (IL-21), interleukin-7 (IL-7), or interferon gamma. In some embodiments, the cytokine molecule is a proinflammatory cytokine.

In certain embodiments, the cytokine is a single chain cytokine. In certain embodiments, the cytokine is a multichain cytokine (e.g., the cytokine comprises 2 or more (e.g., 2) poly peptide chains. An exemplary multichain cytokine is IL-12.

Examples of useful cytokines include, but are not limited to, GM-CSF, IL-1α, IL-1β, IL-2, IL-3, IL4, IL-5, IL-6, IL-7, IL-8, IL-10, IL-12, IL-21, IFN-α, IFN-β, IFN-γ, MIP-1α, MIP-1β, TGF-β, TNF-α, and TNFβ. In some embodiments the cytokine of the multispecific or multifunctional polypeptide is a cytokine selected from the group of GM-CSF, IL-2, IL-7, IL-8, IL-10, IL-12, IL-15, IL-21, IFN-α, IFN-γ, MIP-1α, MIP-1β and TGF-β. In some embodiments the cytokine of the 1 the multispecific or multifunctional polypeptide is a cytokine selected from the group of IL-2, IL-7, IL-10, IL-12, IL-15, IFN-α, and IFN-γ. In certain embodiments the cytokine is mutated to remove N- and/or O-glycosylation sites. Elimination of glycosylation increases homogeneity of the product obtainable in recombinant production.

In some embodiments, the cytokine of the multispecific or multifunctional polypeptide is IL-2. In a specific embodiment, the IL-2 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity. In another particular embodiment the 1L-2 cytokine is a mutant IL-2 cytokine having reduced binding affinity to the .alpha.-subunit of the IL-2 receptor. Together with the .beta.- and .gamma.-subunits (also known as CD122 and CD132, respectively), the .alpha.-subunit (also known as CD25) forms the heterotrimeric high-affinity IL-2 receptor, while the dimeric receptor consisting only of the β- and γ-subunits is termed the intermediate-affinity IL-2 receptor. As described in PCT patent application number PCT/EP2012/051991, which is incorporated herein by reference in its entirety, a mutant IL-2 polypeptide with reduced binding to the .alpha.-subunit of the IL-2 receptor has a reduced ability to induce IL-2 signaling in regulatory T cells, induces less activation-induced cell death (AICD) in T cells, and has a reduced toxicity profile in vivo, compared to a wild-type IL-2 polypeptide. The use of such an cytokine with reduced toxicity is particularly advantageous in a multispecific or multifunctional polypeptide according to the invention, having a long serum half-life due to the presence of an Fc domain. In some embodiments, the mutant IL-2 cytokine of the multispecific or multifunctional polypeptide according to the invention comprises at least one amino acid mutation that reduces or abolishes the affinity of the mutant IL-2 cytokine to the .alpha.-subunit of the IL-2 receptor (CD25) but preserves the affinity of the mutant IL-2 cytokine to the intermediate-affinity IL-2 receptor (consisting of the β and γ subunits of the IL-2 receptor), compared to the non-mutated IL-2 cytokine. In some embodiments the one or more amino acid mutations are amino acid substitutions. In a specific embodiment, the mutant IL-2 cytokine comprises one, two or three amino acid substitutions at one, two or three position(s) selected from the positions corresponding to residue 42, 45, and 72 of human IL-2. In a more specific embodiment, the mutant IL-2 cytokine comprises three amino acid substitutions at the positions corresponding to residue 42, 45 and 72 of human IL-2. In an even more specific embodiment, the mutant IL-2 cytokine is human IL-2 comprising the amino acid substitutions F42A. Y45A and L72G. In some embodiments the mutant IL-2 cytokine additionally comprises an amino acid mutation at a position corresponding to position 3 of human IL-2, which eliminates the O-glycosylation site of IL-2. Particularly, said additional amino acid mutation is an amino acid substitution replacing a threonine residue by an alanine residue. A particular mutant IL-2 cytokine useful in the invention comprises four amino acid substitutions at positions corresponding to residues 3, 42, 45 and 72 of human IL-2. Specific amino acid substitutions are T3A, F42A, Y45A and L72G. As demonstrated in PCT patent application number PCT/EP2012/051991 and in the appended Examples, said quadruple mutant IL-2 polypeptide (IL-2 qm) exhibits no detectable binding to CD25, reduced ability to induce apoptosis in T cells, reduced ability to induce IL-2 signaling in T.sub.reg cells, and a reduced toxicity profile in vivo. However, it retains ability to activate IL-2 signaling in effector cells, to induce proliferation of effector cells, and to generate IFN-γ as a secondary cytokine by NK cells.

The IL-2 or mutant IL-2 cytokine according to any of the above embodiments may comprise additional mutations that provide further advantages such as increased expression or stability. For example, the cysteine at position 125 may be replaced with a neutral amino acid such as alanine, to avoid the formation of disulfide-bridged IL-2 dimers. Thus, in certain embodiments the IL-2 or mutant IL-2 cytokine of the multispecific or multifunctional polypeptide according to the invention comprises an additional amino acid mutation at a position corresponding to residue 125 of human IL-2. In some embodiments said additional amino acid mutation is the amino acid substitution C125A.

In a specific embodiment the IL-2 cytokine of the multispecific or multifunctional polypeptide comprises the polypeptide sequence of SEQ ID NO: 2270

[APTSSSTKKTQLQLEHLLLDLQMILNGINNYKNPKLTRMLTFKFYMPK
KATELKHLQCLEEELKPLEEVLNLAQSKNFHLRPRDLISNINVIVLELK
GSETTFMCEYADETATIVEFLNRWITFAQSISTLT].

In another specific embodiment the IL-2 cytokine of the multispecific or multifunctional polypeptide comprises the polypeptide sequence of SEQ ID NO: 2280

[APASSSTKKTQLQLEHLLLDLQMILNGINNYKNPKLTRMLTAKFAMPK
KATELKHLQCLEEELKPLEEVLNGAQSKNFHLRPRDLISNINVIVLELK
GSETTFMCEYADETATIVEFLNRWITFAQSIISTLT].

In another embodiment the cytokine of the multispecific or multifunctional polypeptide is IL-12. In a specific embodiment said IL-12 cytokine is a single chain IL-12 cytokine. In an even more specific embodiment the single chain IL-12 cytokine comprises the polypeptide sequence of SEQ ID NO: 2290

[IWELKKDVYVVELDWYPDAPGEMVVLTCDTPEEDGITWTLDQSSEVLGSGKTLTIQVKEFGDA
GQYTCHKGGEVLSHSLLLLHKKEDGIWSTDILKDQKEPKNKTFLRCEAKNYSGRFTCWWLTTIS
TDLTFSVKSSRGSSDPQGVTCGAATLSAERVRGDNKEYEYSVECQEDSACPAAEESLPIEVMVDA
VHKLKYENYTSSFFIRDIIKPDPPKNLQLKPLKNSRQVEVSWEYPDTWSTPHSYFSLTFCVQVQGK
SKREKKDRVFTDKTSATVICRKNASISVRAQDRYYSSSWSEWASVPCSGGGGSGGGGSGGGGSR
NLPVATPDPGMFPCLHHSQNLLRAVSNMLQKARQTLEFYPCTSEEIDHEDITKDKTSTVEACLPL
ELTKNESCLNSRETSFITNGSCLASRKTSFMMALCLSSIYEDLKMYQVEFKTMNAKLLMDPKRQI
FLDQNMLAVIDELMQALNFNSETVPQKSSLEEPDFYKTKIKLCILLHAFRIRAVTIDRVMSYLNAS]

In some embodiments, the IL-12 cytokine can elicit one or more of the cellular responses selected from the group consisting of: proliferation in a NK cell, differentiation in a NK cell, proliferation in a T cell, and differentiation in a T cell.

In another embodiment the cytokine of the multispecific or multifunctional polypeptide is IL-10. In a specific embodiment said IL-10 cytokine is a single chain IL-10 cytokine. In an even more specific embodiment the single chain IL-10 cytokine comprises the polypeptide sequence of SEQ ID NO: 2300

[SPGQGTQSENSCTHFPGNLPNMLRDLRDAFSRVKTFFQMKDQLDNLLLKESLLEDFKGYLGCQ
ALSEMIQFYLEEVMPQAENQDPDIKAHVNSLGENLKTLRLRLRRCHRFLPCENKSKAVEQVKNA
FNKLQEKGIYKAMSEFDIFINYIEAYMTMKIRNGGGGSGGGGSGGGGSGGGGSSPGQGTQSENSC
THFPGNLPNMLRDLRDAFSRVKTFFQMKDQLDNLLLKESLLEDFKGYLGCQALSEMIQFYLEEV
MPQAENQDPDIKAHVNSLGENLKTLRLRLRRCHRFLPCENKSKAVEQVKNAFNKLQEKGIYKA
MSEFDIFINYIEAYMTMKIRN].

In another specific embodiment the IL-10 cytokine is a monomeric IL-10 cytokine. In a more specific embodiment the monomeric IL-10 cytokine comprises the polypeptide sequence of SEQ ID NO: 2310 [SPGQGTQSENSCTHFPGNLPNMLRDLRDAFSRVKTFFQMKDQLDNLLLKESLLEDFKGYLGCQ ALSEMIQFYLEEVMPQAENQDPDIKAHVNSLGENLKTLRLRLRRCHRFLPCENGGGSGGKSKAV EQVKNAFNKLQEKGIYKAMSEFDIFINYIEAYMTMKIRN]. In some embodiments, the IL-10 cytokine can elicit one or more of the cellular responses selected from the group consisting of: inhibition of cytokine secretion, inhibition of antigen presentation by antigen presenting cells, reduction of oxygen radical release, and inhibition of T cell proliferation. A multispecific or multifunctional polypeptide according to the invention wherein the cytokine is IL-10 is particularly useful for downregulation of inflammation, e.g. in the treatment of an inflammatory disorder.

In another embodiment, the cytokine of the multispecific or multifunctional polypeptide is IL-15. In a specific embodiment said IL-15 cytokine is a mutant IL-15 cytokine having reduced binding affinity to the α-subunit of the IL-15 receptor. Without wishing to be bound by theory, a mutant IL-15 polypeptide with reduced binding to the .alpha.-subunit of the IL-15 receptor has a reduced ability to bind to fibroblasts throughout the body, resulting in improved pharmacokinetics and toxicity profile, compared to a wild-type IL-15 polypeptide. The use of an cytokine with reduced toxicity, such as the described mutant IL-2 and mutant IL-15 effector moieties, is particularly advantageous in a multispecific or multifunctional polypeptide according to the invention, having a long serum half-life due to the presence of an Fc domain. In some embodiments the mutant IL-15 cytokine of the multispecific or multifunctional polypeptide according to the invention comprises at least one amino acid mutation that reduces or abolishes the affinity of the mutant IL-15 cytokine to the .alpha.-subunit of the IL-15 receptor but preserves the affinity of the mutant IL-15 cytokine to the intermediate-affinity IL-15/IL-2 receptor (consisting of the .beta.- and .gamma.-subunits of the IL-15/IL-2 receptor), compared to the non-mutated IL-15 cytokine. In some embodiments the amino acid mutation is an amino acid substitution. In a specific embodiment, the mutant IL-15 cytokine comprises an amino acid substitution at the position corresponding to residue 53 of human IL-15. In a more specific embodiment, the mutant IL-15 cytokine is human IL-15 comprising the amino acid substitution E53A. In some embodiments the mutant IL-15 cytokine additionally comprises an amino acid mutation at a position corresponding to position 79 of human IL-15, which eliminates the N-glycosylation site of IL-15. Particularly, said additional amino acid mutation is an amino acid substitution replacing an asparagine residue by an alanine residue. In an even more specific embodiment the IL-15 cytokine comprises the polypeptide sequence of SEQ ID NO: 2320 [NWVNVISDLKKIEDLIQSMHIDATLYTESDVHPSCKVTAMKCFLLELQVISLASGDASIHDTVEN LIILANNSLSSNGAVTESGCKECEELEEKNIKEFLQSFVHIVQMFINTS]. In some embodiments, the IL-15 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

Mutant cytokine molecules useful as effector moieties in the multispecific or multifunctional polypeptide can be prepared by deletion, substitution, insertion or modification using genetic or chemical methods well known in the art. Genetic methods may include site-specific mutagenesis of the encoding DNA sequence. PCR, gene synthesis, and the like. The correct nucleotide changes can be verified for example by sequencing. Substitution or insertion may involve natural as well as non-natural amino acid residues. Amino acid modification includes well known methods of chemical modification such as the addition or removal of glycosylation sites or carbohydrate attachments, and the like.

In some embodiments, the cytokine, particularly a single-chain cytokine, of the multispecific or multifunctional polypeptide is GM-CSF. In a specific embodiment, the GM-CSF cytokine can elicit proliferation and/or differentiation in a granulocyte, a monocyte or a dendritic cell. In some embodiments, the cytokine, particularly a single-chain cytokine, of the multispecific or multifunctional polypeptide is IFN-α. In a specific embodiment, the IFN-α cytokine can elicit one or more of the cellular responses selected from the group consisting of: inhibiting viral replication in a virus-infected cell, and upregulating the expression of major histocompatibility complex I (MHC I). In another specific embodiment, the IFN-α cytokine can inhibit proliferation in a tumor cell. In some embodiments the cytokine, particularly a single-chain cytokine, of the multispecific or multifunctional polypeptide is IFNγ. In a specific embodiment, the IFN-γ cytokine can elicit one or more of the cellular responses selected from the group of: increased macrophage activity, increased expression of MHC molecules, and increased NK cell activity. In some embodiments the cytokine, particularly a single-chain cytokine, of the multispecific or multifunctional polypeptide is IL-7. In a specific embodiment, the IL-7 cytokine can elicit proliferation of T and/or B lymphocytes. In some embodiments, the cytokine, particularly a single-chain cytokine, of the multispecific or multifunctional polypeptide is IL-8. In a specific embodiment, the IL-8 cytokine can elicit chemotaxis in neutrophils. In some embodiments, the cytokine, particularly a single-chain cytokine, of the multispecific or multifunctional polypeptide, is MIP-1α. In a specific embodiment, the MIP-1α cytokine can elicit chemotaxis in monocytes and T lymphocyte cells. In some embodiments, the cytokine, particularly a single-chain cytokine, of the multispecific or multifunctional polypeptide is MIP-1β. In a specific embodiment, the MIP-1β cytokine can elicit chemotaxis in monocytes and T lymphocyte cells. In some embodiments, the cytokine, particularly a single-chain cytokine, of the multispecific or multifunctional polypeptide is TGF-β. In a specific embodiment, the TGF-β cytokine can elicit one or more of the cellular responses selected from the group consisting of: chemotaxis in monocytes, chemotaxis in macrophages, upregulation of IL-1 expression in activated macrophages, and upregulation of IgA expression in activated B cells.

In some embodiments, the multispecific or multifunctional polypeptide of the invention binds to an cytokine receptor with a dissociation constant (K_{D}) that is at least about 1, 1.5, 2, 2.5, 3, 3.5, 4, 4.5, 5, 5.5, 6, 6.5, 7, 7.5, 8, 8.5, 9, 9.5 or 10 times greater than that for a control cytokine. In another embodiment, the multispecific or multifunctional polypeptide binds to an cytokine receptor with a K_{D} that is at least 2, 3, 4, 5, 6, 7, 8, 9, or 10 times greater than that for a corresponding multispecific or multifunctional polypeptide comprising two or more effector moieties. In another embodiment, the multispecific or multifunctional polypeptide binds to an cytokine receptor with a dissociation constant K_{D} that is about 10 times greater than that for a corresponding the multispecific or multifunctional polypeptide comprising two or more cytokines.

In some embodiments, the multispecific molecules as described herein include a cytokine molecule. In embodiments, the cytokine molecule includes a full length, a fragment or a variant of a cytokine; a cytokine receptor domain, e.g., a cytokine receptor dimerizing domain; or an agonist of a cytokine receptor, e.g., an antibody molecule (e.g., an agonistic antibody) to a cytokine receptor.

In some embodiments the cytokine molecule is chosen from IL-2, IL-12, IL-15, IL-18, IL-7, IL-21, or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain.

In other embodiments, the cytokine molecule is an agonist of a cytokine receptor, e.g., an antibody molecule (e.g., an agonistic antibody) to a cytokine receptor chosen from an IL-15Ra or IL-21R.

In some embodiments, the cytokine molecule is IL-15, e.g., human 1L-15 (e.g., comprising the amino acid sequence: NWVNVISDLKKIEDLIQSMHIDATLYTESDVHPSCKVTAMKCFLLELQVISLESGDASIHDTVENL IILANNSLSSNGNVTESGCKECEELEEKNIKEFLQSFVHIVQMFINTS (SEQ ID NO: 2170), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2170.

In some embodiments, the cytokine molecule comprises a receptor dimerizing domain, e.g., an IL15Ralpha dimerizing domain. In some embodiments, the IL15Ralpha dimerizing domain comprises the amino acid sequence: MAPRRARGCRTLGLPALLLLLLLRPPATRGITCPPPMSVEHADIWVKSYSLYSRERYICNSGFKRK AGTSSLTECVL (SEQ ID NO: 2180), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2180. In some embodiments, the cytokine molecule (e.g., IL-15) and the receptor dimerizing domain (e.g., an IL15Ralpha dimerizing domain) of the multispecific molecule are covalently linked, e.g., via a linker (e.g., a Gly-Ser linker, e.g., a linker comprising the amino acid sequence SGGSGGGGSGGGSGGGGSLQ (SEQ ID NO: 2190). In other embodiments, the cytokine molecule (e.g., IL-15) and the receptor dimerizing domain (e.g., an IL15Ralpha dimerizing domain) of the multispecific molecule are not covalently linked, e.g., are non-covalently associated.

In other embodiments, the cytokine molecule is IL-2, e.g., human IL-2 (e.g., comprising the amino acid sequence: APTSSSTKKTQLQLEHLLLDLQMILNGINNYKNPKLTRMLTFKFYMPKKATELKHLQCLEEELKP LEEVLNLAQSKNFHLRPRDLISNINVIVLELKGSETTFMCEYADETATIVEFLNRWITFCQSIISTLT (SEQ ID NO: 2191), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO:2191).

In other embodiments, the cytokine molecule is IL-18, e.g., human IL-18 (e.g., comprising the amino acid sequence: YFGKLESKLSVIRNLNDQVLFIDQGNRPLFEDMTDSDCRDNAPRTIFIISMYKDSQPRGMAVTISV KCEKISTLSCENKIISFKEMNPPDNIKDTKSDITFFQRSVPGHDNKMQFESSSYEGYFLACEKERDL FKLILKKEDELGDRSIMFTVQNED (SEQ ID NO: 2192), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions. e.g., conservative substitutions) to the amino acid sequence of SEQ ID NO: 2192).

In other embodiments, the cytokine molecule is IL-21, e.g., human IL-21 (e.g., comprising the amino acid sequence: QGQDRHMIRMRQLIDIVDQLKNYVNDLVPEFLPAPEDVETNCEWSAFSCFQKAQLKSANTGNNE RTNVSIKKLKRKPPSTNAGRRQKHRLTCPSCDSYEKKPPKEFLERFKSLLQKMIHQHLSSRTHGSE DS (SEQ ID NO: 2193), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2193).

In yet other embodiments, the cytokine molecule is interferon gamma, e.g., human interferon gamma (e.g., comprising the amino acid sequence: QDPYVKEAENLKKYFNAGHSDVADNGTLFLGILKNWKEESDRKIMQSQIVSFYFKLFKNFKDDQ SIQKSVETIKEDMNVKFFNSNKKKRDDFEKLTNYSVTDLNVQRKAIHELIQVMAELSPAAKTGKR KRSQMLFRG (SEQ ID NO: 2194), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2194).

### Immune Cell Engagers

In some embodiments, the multifunctional molecule further includes an immune cell engager. “An immune cell engager” refers to one or more binding specificities that bind and/or activate an immune cell, e.g., a cell involved in an immune response. In embodiments, the immune cell is chosen from a T cell, an NK cell, a B cell, a dendritic cell, and/or the macrophage cell. The immune cell engager can be an antibody molecule, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to the immune cell antigen (e.g., the T cell, the NK cell antigen, the B cell antigen, the dendritic cell antigen, and/or the macrophage cell antigen). In embodiments, the immune cell engager specifically binds to the target immune cell, e.g., binds preferentially to the target immune cell. For example, when the immune cell engager is an antibody molecule, it binds to an immune cell antigen (e.g., a T cell antigen, an NK cell antigen, a B cell antigen, a dendritic cell antigen, and/or a macrophage cell antigen) with a dissociation constant of less than about 10 nM.

The immune cell engagers, e.g., first and/or second immune cell engager, of the multispecific or multifunctional molecules as described herein can mediate binding to, and/or activation of, an immune cell, e.g., an immune effector cell. In some embodiments, the immune cell is chosen from a T cell, an NK cell, a B cell, a dendritic cell, or a macrophage cell engager, or a combination thereof. In some embodiments, the immune cell engager is chosen from one, two, three, or all of a T cell engager. NK cell engager, a B cell engager, a dendritic cell engager, or a macrophage cell engager, or a combination thereof. The immune cell engager can be an agonist of the immune system. In some embodiments, the immune cell engager can be an antibody molecule, a ligand molecule (e.g., a ligand that further comprises an immunoglobulin constant region, e.g., an Fc region), a small molecule, a nucleotide molecule.

### Natural Killer Cell Engagers:

Natural Killer (NK) cells recognize and destroy tumors and virus-infected cells in an antibody-independent manner. The regulation of NK cells is mediated by activating and inhibiting receptors on the NK cell surface. One family of activating receptors is the natural cytotoxicity receptors (NCRs) which include NKp30, NKp44 and NKp46. The NCRs initiate tumor targeting by recognition of heparan sulfate on cancer cells. NKG2D is a receptor that provides both stimulatory and costimulatory innate immune responses on activated killer (NK) cells, leading to cytotoxic activity. DNAM1 is a receptor involved in intercellular adhesion, lymphocyte signaling, cytotoxicity and lymphokine secretion mediated by cytotoxic T-lymphocyte (CTL) and NK cell. DAP10 (also known as HCST) is a transmembrane adapter protein which associates with KLRK1 to form an activation receptor KLRK1-HCST in lymphoid and myeloid cells; this receptor plays a major role in triggering cytotoxicity against target cells expressing cell surface ligands such as MHC class I chain-related MICA and MTCB, and U(optionally L1)6-binding proteins (ULBPs); it KLRK1-HCST receptor plays a role in immune surveillance against tumors and is required for cytolysis of tumors cells, indeed, melanoma cells that do not express KLRK1 ligands escape from immune surveillance mediated by NK cells, CD16 is a receptor for the Fc region of IgG, which binds complexed or aggregated IgG and also monomeric IgG and thereby mediates antibody-dependent cellular cytotoxicity (ADCC) and other antibody.-dependent responses, such as phagocytosis.

In some embodiments, the NK cell engager is a viral hemagglutinin (HA). HA is a glycoprotein found on the surface of influenza viruses. It is responsible for binding the virus to cells with sialic acid on the membranes, such as cells in the upper respiratory tract or erythrocytes. HA has at least 18 different antigens. These subtypes are named H1 through H18. NCRs can recognize viral proteins. NKp46 has been shown to be able to interact with the HA of influenza and the HA-NA of Paramyxovirus, including Sendai virus and Newcastle disease virus. Besides NKp46, NKp44 can also functionally interact with HA of different influenza subtypes.

Provided herein are, inter alia, multispecific (e.g., bi-, tri-, quad-specific) or multifunctional molecules that are engineered to contain one or more NK cell engagers that mediate binding to and/or activation of an NK cell. Accordingly, in some embodiments, the NK cell engager is selected from an antigen binding domain or ligand that binds to (e.g., activates): NKp30, NKp40, NKp44, NKp46. NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7. KIR2DS2, KIR2DS4, KIR3DS1, KIR2DS3, KIR2DS5, KIR2DS1, CD94, NKG2C, NKG2E, or CD160.

In some embodiments, the NK cell engager is a ligand of NKp30 is a B7-6, e.g., comprises the amino acid sequence of: DLKVEMMAGGTQITPLNDNVTIFCNIFYSQPLNITSMGITWFWKSLTFDKEVKVFEFFGDHQEAF RPGAIVSPWRLKSGDASLRLPGIQLEEAGEYRCEVVVTPLKAQGTVQLEVVASPASRLLLDQVG MKENEDKYMCESSGFYPEAINITWEKQTQKFPHPIEISEDVITGPTIKNMDGTFNVTSCLKLNSSQ EDPGTVYQCVVRHASLHTPLRSNFTLTAARHSLSETEKTDNFS (SEQ ID NO: 3291), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3291.

In other embodiments, the NK cell engager is a ligand of NKp44 or NKp46, which is a viral HA. Viral hemagglutinins (HA) are glyco proteins which are on the surface of viruses. HA proteins allow viruses to bind to the membrane of cells via sialic acid sugar moieties which contributes to the fusion of viral membranes with the cell membranes (see e.g., Eur J Inumunol. 2001 September; 31(9):2680-9 “Recognition of viral hemagglutinins by NKp44 but not by NKp30”; and Nature. 2001 Feb. 22; 409(6823):1055-60 “Recognition of haemagglutinins on virus-infected cells by NKp46 activates lysis by human NK cells” the contents of each of which are incorporated by reference herein).

In other embodiments, the NK cell engager is a ligand of NKG2D chosen from MICA. MICB, or ULBP1, e.g., wherein: (i) MICA comprises the amino acid sequence: EPHSLRYNLTVLSWDGSVQSGFLTEVHLDGQPFLRCDRQKCRAKPQGQWAEDVLGNKTWDRET RDLTGNGKDLRMTLAHIKDQKEGLHSLQEIRVCEIHEDNSTRSSQHFYYDGELFLSQNLETKEWT MPQSSRAQTLAMNVRNFLKEDAMKTKTHYHAMHADCLQELRRY LKSGVVLRRTVPPMVNVTR SEASEGNITVTCRASGFYPWNITLSWRQDGVSLSHDTQQWGDVLPDGNGTYQTWVATRICQGEE QRFTCYMEHSGNHSTHPVPSGKVLVLQSHW (SEQ ID NO: 3292), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3292; (ii) MICB comprises the amino acid sequence: AEPHSLR YNLMVLSQDESVQSGFL AEGHLDGQPFLRYDRQKRRAKPQGQWAEDVLGAKTWDT ETEDLTENGQDLRRTLTHIKDQKGGLHSLQEIRVCEIHEDSSTRGSRHFYYDGELFLSQNLETQES TVPQSSRAQTLAMNVTNFWKEDAMKTKTHYRAMQADCLQKLQRYLKSGVAIRRTVPPMVNVT CSEVSEGNITVTCRASSFYPRNITLTWRQDGVSLSHNTQQWGDVLPDGNGTYQTWVATRIRQGE EQRFTCYMEHSGNHGTHPVPSGKVLVLQSQRTD (SEQ ID NO: 3293), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3293; or (iii) ULBP1 comprises the amino acid sequence: GWVDTHCLCYDFIITPKSRPEPQWCEVQGLVDERPFLHYDCVNHKAKAFASLGKKVNVTKTWE EQTETLRDVVDFLKGQLLDIQVENLIPIEPLTLQARMSCEHEAHGHGRGSWQFLFNGQKFLLFDS NNRKWTALHPGAKKMTEKWEKNRDVTMFFQKISLGDCKMWLEEFLMYWEQMLDPTKPPSLAP G (SEQ ID NO: 3294), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3294.

In other embodiments, the NK cell engager is a ligand of DNAM I chosen from NECTIN2 or NECL5, e.g., wherein: (i) NECTIN2 comprises the amino acid sequence: QDVRVQVLPEVRGQLGGTVELPCHLLPPVPGLYISLVTWQRPDAPANHQNVAAFHPKMGPSFPS PKPGSERLSFVSAKQSTGQDTEAELQDATLALHGLTVEDEGNYTCEFATFPKGSVRGMTWLRVI AKPKNQAEAQKVTFSQDPTTVALCISKEGRPPARISWLSSLDWEAKETQVSGTLAGTVTVTSRFT LVPSGRADGVTVTCKVEHESFEEPALIPVTLSVRYPPEVSISGYDDNWYLGRTDATLSCDVRSNP EPTGYDWSTTSGTFPTSAVAQGSQLVIHAVDSLFNTTFVCTVTNAVGMGRAEQVIFVRETPNTAG AGATGG (SEQ ID NO: 3295), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3295; or (ii) NECL5 comprises the amino acid sequence: WPPPGTGDVVVQAPTQVPGFLGDSVTLPCYLQVPNMEVTHVSQLTW ARHGESGSMAVFHQTQ GPSYSESKRLEFVAARLGAELRNASLRMFGLRVEDEGNYTCLFVTFPQGSRSVDIWLRVLAKPQ NTAEVQKVQLTGEPVPMARCVSTGGRPPAQITWHSDLGGMPNTSQVPGFLSGTVTVTSLWILVP SSQVDGKNVTCKVEHESFEKPQLLTVNLTVYYPPEVSISGYDNNWYLGQNEATLTCDARSNPEP TGYNWSTTMGPLPPFAVAQGAQLLIRPVDKPINTTLICNVTNALGARQAELTVQVKEGPPSEHSG ISRN (SEQ ID NO: 3296), a fragment thereof, or an amino acid sequence substantially identical thereto (e.g., 95% to⁹⁹0.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3296.

In yet other embodiments, the NK cell engager is a ligand of DAP10, which is an adapter for NKG2D (see e.g., Proc Natl Acad Sci USA. 2005 May 24: 102(21): 7641-7646; and Blood. 15 Sep. 2011 Volume 118, Number 11, the full contents of each of which is incorporated by reference herein).

In other embodiments, the NK cell engager is a ligand of CD16, which is a CD16a/b ligand, e.g., a CD16a/b ligand further comprising an antibody Fc region (see e.g., Front Immunol. 2013; 4: 76 discusses how antibodies use the Fc to trigger NK cells through CD16, the full contents of which are incorporated herein).

In other embodiments, the NK cell engager is a ligand of CRTAM, which is NECL2, e.g., wherein NECL2 comprises the amino acid sequence: QNLFTKDVTVIEGEVATISCQVNKSDDSVIQLLNPNRQTIYFRDFRPLKDSRFQLLNFSSSELKVSL TNVSISDEGRYFCQLYTDPPQESYTTITVLVPPRNLMIDIQKDTAVEGEEIEVNCTAMASKPATTIR WFKGNTELKGKSEVEEWSDMYTVTSQLMLKVHKEDDGVPVICQVEHPAVTGNLQTQRYLEVQ YKPQVHIQMTYPLQGLTREGDALELTCEAIGKPQPVMVTWVRVDDEMPQHAVLSGPNLFINNLN KTDNGTYRCEASNIVGKAHSDYMLYVYDPPTTIPPPTTTTTTTTTTTTTTLTIITDSRAGEEGSIRAV DH (SEQ ID NO: 3297), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3297.

In other embodiments, the NK cell engager is a ligand of CD27, which is CD70, e.g., wherein CD70 comprises the amino acid sequence: QRFAQAQQQLPLESLGWDVAELQLNHTGPQQDPRLYWQGGPALGRSFLHGPELDKGQLRIHRD GIYMVHIQVTLAICSSTTASRHHPTTLAVGICSPASRSISLLRLSFHQGCTIASQRLTPLARGDTLCT NLTGTLLPSRNTDETFFGVQWVRP (SEQ ID NO: 3298), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions. e.g., conservative substitutions) to the amino acid sequence of SEQ ID NO: 3298.

In other embodiments, the NK cell engager is a ligand of PSGL1, which is L-selectin (CD62L), e.g., wherein L-selectin comprises the amino acid sequence: WTYHYSEKPMNWQRARRFCRDNYTDLVAIQNKAEIEYLEKTLPFSRSYYWIGIRKIGGIWTWVG TNKSLTEEAENWGDGEPNNKKNKEDCVEIYIKRNKDAGKWNDDACHKLKAALCYTASCQPWS CSGHGECVEIINNYTCNCDVGYYGPQCQFVIQCEPLEAPELGTMDCTHPLGNFSFSSQCAFSCSEG TNLTGIEETTCGPFGNWSSPEPTCQVIQCEPLSAPDLGIMNCSHPLASFSFTSACTFICSEGTELIGK KKTICESSGIWSNPSPICQKLDKSFSMIKEGDYN (SEQ ID NO: 3299), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3299.

In other embodiments, the NK cell engager is a ligand of CD96, which is NECL5, e.g., wherein NECL5 comprises the amino acid sequence: WPPPGTGDVVVQAPTQVPGFLGDSVTLPCYLQVPNMEVTHVSQLTWARHGESGSMAVFHQTQ GPSYSESKRLEFVAARLGAELRNASLRMFGLRVEDEGNYTCLFVTFPQGSRSVDIWLRVLAKPQ NTAEVQKVQLTGEPVPMARCVSTGGRPPAQITWHSDLGGMPNTSQVPGFLSGTVTVTSLWILVP SSQVDGKNVTCKVEHESFEKPQLLTVNLTVYYPPEVSISGYDNNWYLGQNEATLTCDARSNPEP TGYNWSTTMGPLPPFAVAQGAQLLIRPVDKPIN TTLICNVTNALGARQAELTVQVKEGPPSEHSG ISRN (SEQ ID NO: 3296), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3296.

In other embodiments, the NK cell engager is a ligand of CD100 (SEMA4D), which is CD72, e.g., wherein CD72 comprises the amino acid sequence: RYLQVSQQLQQTNRVLEVTNSSLRQQLRLKITQLGQSAEDLQGSRRELAQSQEALQVEQRAHQA AEGQLQACQADRQKTKETLQSEEQQRRALEQKLSNMENRLKPFFTCGSADTCCPSGWIMHQKS CFYISLTSKNWQESQKQCETLSSKLATFSEIYPQSHSYYFLNSLLPNGGSGNSYWTGLSSNKDWK LTDDTQRTRTYAQSSKCNKVHKTWSWWTLESESCRSSLPYICEMTAFRFPD (SEQ ID NO: 3300), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3300.

In other embodiments, the NK cell engager is a ligand of NKp80, which is CLEC2B (AICL), e.g., wherein CLEC2B (AICL) comprises the amino acid sequence: KLTRDSQSLCPYDWIGFQNKCYYFSKEEGDWNSSKYNCSTQHADLTIIDNIEEMNFLRRYKCSSD HWIGLKMAKNRTGQWVDGATFTKSFGMRGSEGCAYLSDDGAATARCYTERKWICRKRIH (SEQ ID NO: 3301), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3301.

In other embodiments, the NK cell engager is a ligand of CD244, which is CD48, e.g., wherein CD48 comprises the amino acid sequence: QGHLVHMTVVSGSNVTLNISESLPENYKQLTWFYTFDQKIVEWDSRKSKYFESKFKGRVRLDPQ SGALYISKVQKEDNSTYIMRVLKKTGNEQEWKIKLQVLDPVPKPVIKIEKIEDMDDNCYLKLSCV IPGESVNYTWYGDKRPFPKELQNSVLETTLMPHNYSRCYTCQVSNSVSSKNGTVCLSPPCTLARS (SEQ ID NO: 3302), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3302.

### T Cell Engagers

Provided herein are, inter aha, multispecific (e.g., bi-, tri-, quad-specific) or multifunctional molecules that are engineered to further contain one or more T cell engager that mediate binding to and/or activation of a T cell. In some embodiments, the T cell engager is an antigen binding domain that binds to, e.g., activates TCRβ, e.g., a TCRβ 3V region, as described herein. In some embodiments, the T cell engager is selected from an antigen binding domain or ligand that binds to (e.g., and in some embodiments activates) one or more of CD3, TCRα, TCRγ, TCRζ, ICOS, CD28, CD27, HVEM, LIGHT, CD40, 4-1BB, OX40, DR3, GITR, CD30, TIM1, SLAM, CD2, or CD226. In other embodiments, the T cell engager is selected from an antigen binding domain or ligand that binds to and does not activate one or more of CD3, TCRα, TCRγ, TCRξ, ICOS, CD28, CD27, HVEM, LIGHT, CD40, 4-1BB, OX40, DR3, GITR, CD30, TIM1, SLAM, CD2, or CD226.

### B Cell, Macrophage & Dendritic Cell Engagers

Broadly, B cells, also known as B lymphocytes, are a type of white blood cell of the lymphocyte subtype. They function in the humoral immunity component of the adaptive immune system by secreting antibodies. Additionally, B cells present antigen (they are also classified as professional antigen-presenting cells (APCs)) and secrete cytokines. Macrophages are a type of white blood cell that engulfs and digests cellular debris, foreign substances, microbes, cancer cells via phagocytosis. Besides phagocytosis, they play important roles in nonspecific defense (innate immunity) and also help initiate specific defense mechanisms (adaptive immunity) by recruiting other immune cells such as lymphocytes. For example, they are important as antigen presenters to T cells. Beyond increasing inflammation and stimulating the immune system, macrophages also play an important anti-inflammatory role and can decrease immune reactions through the release of cytokines. Dendritic cells (DCs) are antigen-presenting cells that function in processing antigen material and present it on the cell surface to the T cells of the immune system.

Provided herein are, inter alia, multispecific (e.g., bi-, tri-, quad-specific) or multifunctional molecules that further include, e.g., are engineered to contain, one or more B cell, macrophage, and/or dendritic cell engager that mediate binding to and/or activation of a B cell, macrophage, and/or dendritic cell.

Accordingly, in some embodiments, the immune cell engager comprises a B cell, macrophage, and/or dendritic cell engager chosen from one or more of CD40 ligand (CD40L) or a CD70 ligand: an antibody molecule that binds to CD40 or CD70; an antibody molecule to OX40; an OX40 ligand (OX40L); an agonist of a Toll-like receptor (e.g., as described herein, e.g., a TLR4, e.g., a constitutively active TLR4 (caTLR4), or a TLR9 agonists); a 41BB; a CD2; a CD47; or a STING agonist, or a combination thereof.

In some embodiments, the B cell engager is a CD40L, an OX40L, or a CD70 ligand, or an antibody molecule that binds to OX40, CD40 or CD70.

In some embodiments, the macrophage engager is a CD2 agonist. In some embodiments, the macrophage engager is an antigen binding domain that binds to: CD40L or antigen binding domain or ligand that binds CD40, a Toll like receptor (TLR) agonist (e.g., as described herein), e.g., a TLR9 or TLR4 (e.g., caTLR4 (constitutively active TLR4), CD47, or a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In some embodiments, the dendritic cell engager is a CD2 agonist. In some embodiments, the dendritic cell engager is a ligand, a receptor agonist, or an antibody molecule that binds to one or more of: OX40L, 41BB, a TLR agonist (e.g., as described herein) (e.g., TLR9 agonist. TLR4 (e.g., caTLR4 (constitutively active TLR4)), CD47, or and a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In other embodiments, the immune cell engager mediates binding to, or activation of, one or more of a B cell, a macrophage, and/or a dendritic cell. Exemplary B cell, macrophage, and/or dendritic cell engagers can be chosen from one or more of CD40 ligand (CD40L) or a CD70 ligand; an antibody molecule that binds to CD40 or CD70; an antibody molecule to OX40; an OX40 ligand (OX40L); a Toll-like receptor agonist (e.g., a TLR4, e.g., a constitutively active TLR4 (caTLR4) or a TLR9 agonist): a 41BB agonist; a CD2; a CD47; or a STING agonist, or a combination thereof.

In some embodiments, the B cell engager is chosen from one or more of a CD40L, an OX40L, or a CD70 ligand, or an antibody molecule that binds to OX40, CD40 or CD70.

In other embodiments, the macrophage cell engager is chosen from one or more of a CD2 agonist; a CD40L; an OX40L; an antibody molecule that binds to OX40, CD40 or CD70; a Toll-like receptor agonist or a fragment thereof (e.g., a TLR4, e.g., a constitutively active TLR4 (caTLR4)); a CD47 agonist; or a STING agonist.

In other embodiments, the dendritic cell engager is chosen from one or more of a CD2 agonist, an OX40 antibody, an OX40L, 41BB agonist, a Toll-like receptor agonist or a fragment thereof (e.g., a TLR4, e.g., a constitutively active TLR4 (caTLR4)), CD47 agonist, or a STING agonist.

In some embodiments, the OX40L comprises the amino acid sequence: QVSHRYPRIQSIKVQFTEYKKEKGFILTSQKEDEIMKVQNNSVIINCDGFYLISLKGYFSQEVNISL HYQKDEEPLFQLKKVRSVNSLMVASLTYKDKVYLNVTTDNTSLDDFHVNGGELILIHQNPGEFC VL (SEQ ID NO: 3303), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3303.

In another embodiment, the CD40L comprises the amino acid sequence: MQKGDQNPQIAAHVISEASSKTTSVLQWAEKGYYTMSNNLVTLENGKQLTVKRQGLYYIYAQV TFCSNREASSQAPFIASLCLKSPGRFERILLRAANTHSSAKPCGQQSIHLGGVFELQPGASVFVNVT DPSQVSHGTGFTSFGLLKL (SEQ ID NO: 3304), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3304.

In yet other embodiments, the STING agonist comprises a cyclic dinucleotide, e.g., a cyclic di-GMP (cdGMP), a cyclic di-AMP (cdAMP), or a combination thereof, optionally with 2′,5′ or 3′,5′ phosphate linkages.

In some embodiments, the immune cell engager includes 41BB ligand, e.g., comprising the amino acid sequence: ACPWAVSGARASPGSAASPRLREGPELSPDDPAGLLDLRQGMFAQLVAQNVLLIDGPLSWYSDP GLAGVSLTGGLSYKEDTKELVVAKAGVYYVFFQLELRRVVAGEGSGSVSLALHLQPLRSAAGA AALALTVDLPPASSEARNSAFGFQGRLLHLSAGQRLGVHLHTEARARHAWQLTQGATVLGLFR VTPEIPAGLPSPRSE (SEQ ID NO: 3305), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3305.

Toll-Like Receptors: Toll-Like Receptors (TLRs) are evolutionarily conserved receptors are homologues of the Drosophila Toll protein, and recognize highly conserved structural motifs known as pathogen-associated microbial patterns (PAMPs), which are exclusively expressed by microbial pathogens, or danger-associated molecular patterns (DAMPs) that are endogenous molecules released from necrotic or dying cells. PAMPs include various bacterial cell wall components such as lipopolysaccharide (LPS), peptidoglycan (PGN) and lipopeptides, as well as flagellin, bacterial DNA and viral double-stranded RNA. DAMPs include intracellular proteins such as heat shock proteins as well as protein fragments from the extracellular matrix. Stimulation of TLRs by the corresponding PAMPs or DAMPs initiates signaling cascades leading to the activation of transcription factors, such as AP-1, NF-κB and interferon regulatory factors (IRFs). Signaling by TLRs results in a variety of cellular responses, including the production of interferons (IFNs), pro-inflammatory cytokines and effector cytokines that direct the adaptive immune response. TLRs are implicated in a number of inflammatory and immune disorders and play a role in cancer (Rakoff-Nahoum S. & Medzhitov R., 2009. Toll-like receptors and cancer. Nat Revs Cancer 9:57-63).

TLRs are type I transmembrane proteins characterized by an extracellular domain containing leucine-rich repeats (LRRs) and a cytoplasmic tail that contains a conserved region called the Toll/IL-1 receptor (TIR) domain. Ten human and twelve murine TLRs have been characterized, TLR1 to TLR 10 in humans, and TLR1 to TLR9, TLR11, TLR12 and TLR13 in mice, the homolog of TLR10 being a pseudogene. TLR2 is essential for the recognition of a variety of PAMPs from Gram-positive bacteria, including bacterial lipoproteins, lipomannans and lipoteichoic acids. TLR3 is implicated in virus-derived double-stranded RNA. TLR4 is predominantly activated by lipopolysaccharide. TLR5 detects bacterial flagellin and TLR9 is required for response to unmethylated CpG DNA. Finally, TLR7 and TLR8 recognize small synthetic antiviral molecules, and single-stranded RNA was reported to be their natural ligand. TLR11 has been reported to recognize uropathogenic E. coli and a profilin-like protein from Toxoplasma gondii. The repertoire of specificities of the TLRs is apparently extended by the ability of TLRs to heterodimerize with one another. For example, dimers of TLR2 and TLR6 are required for responses to diacylated lipoproteins while TLR2 and TLR1 interact to recognize triacylated lipoproteins. Specificities of the TLRs are also influenced by various adapter and accessory molecules, such as MD-2 and CD14 that form a complex with TLR4 in response to LPS.

TLR signaling consists of at least two distinct pathways: a MyD88-dependent pathway that leads to the production of inflammatory cytokines, and a MyD88-independent pathway associated with the stimulation of IFN-β and the maturation of dendritic cells. The MyD88-dependent pathway is common to all TLRs, except TLR3 (Adachi O. et al., 1998. Targeted disruption of the MyD88 gene results in loss of IL-1- and IL-18-mediated function. Immunity. 9(1):143-50). Upon activation by PAMPs or DAMPs, TLRs hetero- or homodimerize inducing the recruitment of adaptor proteins via the cytoplasmic TIR domain. Individual TLRs induce different signaling responses by usage of the different adaptor molecules. TLR4 and TLR2 signaling requires the adaptor TIRAP/Mal, which is involved in the MyD88-dependent pathway. TLR3 triggers the production of IFN-β in response to double-stranded RNA, in a MyD88-independent manner, through the adaptor TRIF/TICAM-1. TRAM/TICAM-2 is another adaptor molecule involved in the MyD88-independent pathway which function is restricted to the TLR4 pathway.

TLR3, TLR7. TLR8 and TLR9 recognize viral nucleic acids and induce type I IFNs. The signaling mechanisms leading to the induction of type I IFNs differ depending on the TLR activated. They involve the interferon regulatory factors, IRFs, a family of transcription factors known to play a critical role in antiviral defense, cell growth and immune regulation. Three IRFs (IRF3, IRF5 and IRF7) function as direct transducers of virus-mediated TLR signaling. TLR3 and TLR4 activate IRF3 and IRF7, while TLR7 and TLR8 activate IRF5 and IRF7 (Doyle S. et al., 2002. IRF3 mediates a TLR3/TLR4-specific antiviral gene program. Immunity. 17(3):251-63). Furthermore, type I IFN production stimulated by TLR9 ligand CpG-A has been shown to be mediated by PI(3)K and mTOR (Costa-Mattioli M. & Sonenberg N. 2008. RAPping production of type I interferon in pDCs through mTOR. Nature Immunol. 9: 1097-109)).

TLR-9: TLR9 recognizes unmethylated CpG sequences in DNA molecules. CpG sites are relatively rare (˜1%) on vertebrate genomes in comparison to bacterial genomes or viral DNA. TLR9 is expressed by numerous cells of the immune system such as B lymphocytes, monocytes, natural killer (NK) cells, and plasmacytoid dendritic cells. TLR9 is expressed intracellularly, within the endosomal compartments and functions to alert the immune system of viral and bacterial infections by binding to DNA rich in CpG motifs. TLR9 signals leads to activation of the cells initiating pro-inflammatory reactions that result in the production of cytokines such as type-I interferon and IL-12.

TLR Agonists: a TLR agonist can agonize one or more TLR, e.g., one or more of human TLR-1, 2, 3, 4, 5, 6, 7, 8, 9, or 10. In some embodiments, an adjunctive agent described herein is a TLR agonist. In some embodiments, the TLR agonist specifically agonizes human TLR-9. In some embodiments, the TLR-9 agonist is a CpG moiety. As used herein, a CpG moiety, is a linear dinucleotide having the sequence: 5′-C-phosphate-G-3′, that is, cytosine and guanine separated by only one phosphate. In some embodiments, the CpG moiety comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more CpG dinucleotides. In some embodiments, the CpG moiety consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 CpG dinucleotides. In some embodiments, the CpG moiety has 1-5, 1-10, 1-20, 1-30, 1-40, 1-50, 5-10, 5-20, 5-30, 10-20, 10-30, 10-40, or 10-50 CpG dinucleotides. In some embodiments, the TLR-9 agonist is a synthetic ODN (oligodeoxynucleotides). CpG ODNs are short synthetic single-stranded DNA molecules containing unmethylated CpG dinucleotides in particular sequence contexts (CpG motifs). CpG ODNs possess a partially or completely phosphorothioated (PS) backbone, as opposed to the natural phosphodiester (PO) backbone found in genomic bacterial DNA. There are three major classes of CpG ODNs: classes A, B and C, which differ in their immunostimulatory activities. CpG-A ODNs are characterized by a PO central CpG-containing palindromic motif and a PS-modified 3′ poly-G string. They induce high IFN-α production from pDCs but are weak stimulators of TLR9-dependent NF-κB signaling and pro-inflammatory cytokine (e.g, IL-6) production. CpG-B ODNs contain a full PS backbone with one or more CpG dinucleotides. They strongly activate B cells and TLR9-dependent NF-κB signaling but weakly stimulate IFN-α secretion. CpG-C ODNs combine features of both classes A and B. They contain a complete PS backbone and a CpG-containing palindromic motif. C-Class CpG ODNs induce strong IFN-α production from pDC as well as B cell stimulation.

### Stromal Modifying Moieties

In some embodiments, the multifunctional molecule further includes a stromal modifying moiety. A “stromal modifying moiety,” as used herein refers to an agent, e.g., a protein (e.g., an enzyme), that is capable of altering, e.g., degrading a component of, the stroma. In embodiments, the component of the stroma is chosen from, e.g., an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g., collagen, laminin, elastin, fibrinogen, fibronectin, and vitronectin.

Solid tumors have a distinct structure that mimics that of normal tissues and comprises two distinct but interdependent compartments: the parenchyma (neoplastic cells) and the stroma that the neoplastic cells induce and in which they are dispersed. All tumors have stroma and require stroma for nutritional support and for the removal of waste products. In the case of tumors which grow as cell suspensions (e.g., leukemias, ascites tumors), the blood plasma serves as stroma (Connolly J L et al. Tumor Structure and Tumor Stroma Generation. In: Kufe D W et al., editors. Holland-Frei Cancer Medicine. 6th edition. Hamilton: B C Decker; 2003). The stroma includes a variety of cell types, including fibroblasts/myofibroblasts, glial, epithelial, fat, vascular, smooth muscle, and immune cells along with extracellular matrix (ECM) and extracellular molecules (Li Hanchen et al. Tumor Microenvironment: The Role of the Tumor Stroma in Cancer. J of Cellular Biochemistry 101: 805-815 (2007)).

Stromal modifying moieties described herein include moieties (e.g. proteins, e.g., enzymes) capable of degrading a component of the stroma, e.g., an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g., collagen, laminin, elastin, fibrinogen, fibronectin, and vitronectin.

### Stromal Modifying Enzymes

In some embodiments, the stromal modifying moiety is an enzyme. For example, the stromal modifying moiety can include, but is not limited to a hyaluronidase, a collagenase, a chondroitinase, a matrix metalloproteinase (e.g., macrophage metalloelastase).

### Hyaluronidases

Hyaluronidases are a group of neutral- and acid-active enzymes found throughout the animal kingdom. Hyaluronidases vary with respect to substrate specificity, and mechanism of action. There are three general classes of hyaluronidases: (1) Mammalian-type hyaluronidases. (EC 3.2.1.35) which are endo-beta-N-acetylhexosaminidases with tetrasaccharides and hexasaccharides as the major end products. They have both hydrolytic and transglycosidase activities, and can degrade hyaluronan and chondroitin sulfates; (2) Bacterial hyaluronidases (EC 4.2.99.1) degrade hyaluronan and, and to various extents, chondroitin sulfate and dermatan sulfate. They are endo-beta-N-acetylhexosaminidases that operate by a beta elimination reaction that yields primarily disaccharide end products; (3) Hyaluronidases (EC 3.2.1.36) from leeches, other parasites, and crustaceans are endo-beta-glucuronidases that generate tetrasaccharide and hexasaccharide end products through hydrolysis of the beta 1-3 linkage.

Mammalian hyaluronidases can be further divided into two groups: (1) neutral active and (2) acid active enzymes. There are six hyaluronidase-like genes in the human genome, HYAL1, HYAL2, HYAL3 HYAL4 HYALP1 and PH20/SPAM1. HYALP1 is a pseudogene, and HYAL3 has not been shown to possess enzyme activity toward any known substrates. HYAL4 is a chondroitinase and lacks activity towards hyaluronan. HYAL1 is the prototypical acid-active enzyme and PH20 is the prototypical neutral-active enzyme. Acid active hyaluronidases, such as HYAL1 and HYAL2 lack catalytic activity at neutral pH. For example, HYAL1 has no catalytic activity in vitro over pH 4.5 (Frost and Stern, “A Microtiter-Based Assay for Hyaluronidase Activity Not Requiring Specialized Reagents”, Analytical Biochemistry, vol. 251, pp. 263-269 (1997). HYAL2 is an acid active enzyme with a very low specific activity in vitro.

In some embodiments the hyaluronidase is a mammalian hyaluronidase. In some embodiments the hyaluronidase is a recombinant human hyaluronidase. In some embodiments, the hyaluronidase is a neutral active hyaluronidase. In some embodiments, the hyaluronidase is a neutral active soluble hyaluronidase. In some embodiments, the hyaluronidase is a recombinant PH20 neutral-active enzyme. In some embodiments, the hyaluronidase is a recombinant PH20 neutral-active soluble enzyme. In some embodiments the hyaluronidase is glycosylated. In some embodiments, the hyaluronidase possesses at least one N-linked glycan. A recombinant hyaluronidase can be produced using conventional methods known to those of skill in the art, e.g., U.S. Pat. No. 7,767,429, the entire contents of which are incorporated by reference herein.

In some embodiments the hyaluronidase is rHuPH20 (also referred to as Hylenex®; presently manufactured by Halozyme: approved by the FDA in 2005 (see e.g., Scodeller P (2014) Hyaluronidase and other Extracellular Matrix Degrading Enzymes for Cancer Therapy: New Uses and Nano-Formulations. J Carcinog Mutage 5:178; U.S. Pat. Nos. 7,767,429; 8,202,517; 7,431,380; 8,450,470; 8,772,246; 8,580,252, the entire contents of each of which is incorporated by reference herein), rHuPH20 is produced by genetically engineered CHO cells containing a DNA plasmid encoding for a soluble fragment of human hyaluronidase PH20. In some embodiments the hyaluronidase is glycosylated. In some embodiments, the hyaluronidase possesses at least one N-linked glycan. A recombinant hyaluronidase can be produced using conventional methods known to those of skill in the art, e.g., U.S. Pat. No. 7,767,429, the entire contents of which are incorporated by reference herein. In some embodiments, rHuPH20 has a sequence at least 95% (e.g., at least 96%, 97%, 98%, 99%, 100%) identical to the amino acid sequence of

(SEQ ID NO: 3306)
LNFRAPPVIPNVPFLWAWNAPSEFCLGKFDEPLDMSLFSFIGSPRINATGQGVTIFYVDRLGYYPYI
DSITGVTVNGGIPQKISLQDHLDKAKKDITFYMPVDNLGMAVIDWEEWRPTWARNWKPKDVYK
NRSIELVQQQNVOLSLTEATEKAKQEFEKAGKDFLVETIKLGKLLRPNHLWGYYLFPDCYNHHY
KKPGYNGSCFNVEIKRNDDLSWLWNESTALYPSIYLNTQQSPVAATLYVRNRVREAIRVSKIPDA
KSPLPVFAYTRIVFTDQVLKFLSQDELVYTFGETVALGASGIVIWGTLSIMRSMKSCLLLDNYME
TILNPYIINVTLAAKMCSQVLCQEQGVCIRKNWNSSDYLHLNPDNFAIQLEKGGKFTVRGKPTLE
DLEQFSEKFYCSCYSTLSCKEKADVKDTDAVDVCIADGVCIDAFLKPPMETEEPQIFYNASPSTLS.

In any of the methods provided herein, the anti-hyaluronan agent can be an agent that degrades hyaluronan or can be an agent that inhibits the synthesis of hyaluronan. For example, the anti-hyaluronan agent can be a hyaluronan degrading enzyme. In another example, the anti-hyaluronan agent or is an agent that inhibits hyaluronan synthesis. For example, the anti-hyaluronan agent is an agent that inhibits hyaluronan synthesis such as a sense or antisense nucleic acid molecule against an HA synthase or is a small molecule drug. For example, an anti-hyaluronan agent is 4-methylumbelliferone (MU) or a derivative thereof, or leflunomide or a derivative thereof. Such derivatives include, for example, a derivative of 4-methylumbelliferone (MU) that is 6,7-dihydroxy-4-methyl coumarin or 5,7-dihydroxy-4-methyl coumarin.

In further examples of the methods provided herein, the hyaluronan degrading enzyme is a hyaluronidase. In some examples, the hyaluronan-degrading enzyme is a PH20 hyaluronidase or truncated form thereof to lacking a C-terminal glycosylphosphatidylinositol (GPI) attachment site or a portion of the GPI attachment site. In specific examples, the hyaluronidase is a PH20 selected from a human, monkey, bovine, ovine, rat, mouse or guinea pig PH20. For example, the hyaluronan-degrading enzyme is a human PH20 hyaluronidase that is neutral active and N-glycosylated and is selected from among (a) a hyaluronidase polypeptide that is a full-length PH20 or is a C-terminal truncated form of the PH20, wherein the truncated form includes at least amino acid residues 36-464 of SEQ ID NO: 139, such as 36-481, 36-482, 36483, where the full-length PH20 has the sequence of amino acids set forth in SEQ ID NO: 139; or (b) a hyaluronidase polypeptide comprising a sequence of amino acids having at least 85%, 86%, 87%, 88%, 89%, 90%, 91%, 92%, 93%, 94%, 95%, 96%, 97%, 98%, 99% or more sequence identity with the polypeptide or truncated form of sequence of amino acids set forth in SEQ ID NO: 139; or (c) a hyaluronidase polypeptide of (a) or (b) comprising amino acid substitutions, whereby the hyaluronidase poly peptide has a sequence of amino acids having at least 85%, 86%, 87%, 88%, 89%, 90%, 91%, 92%, 93%, 94%, 95%, 96%, 97%, 98%, 99% or more sequence identity with the polypeptide set forth in SEQ ID NO: 139 or the with the corresponding truncated forms thereof. In exemplary examples, the hyaluronan-degrading enzyme is a PH20 that comprises a composition designated rHuPH20.

In other examples, the anti-hyaluronan agent is a hyaluronan degrading enzyme that is modified by conjugation to a polymer. The polymer can be a PEG and the anti-hyaluronan agent a PEGylated hyaluronan degrading enzyme. Hence, in some examples of the methods provided herein the hyaluronan-degrading enzyme is modified by conjugation to a polymer. For example, the hyaluronan-degrading enzyme is conjugated to a PEG, thus the hyaluronan degrading enzyme is PEGylated. In an exemplary example, the hyaluronan-degrading enzyme is a PEGylated PH20 enzyme (PEGPH20). In the methods provided herein, the corticosteroid can be a glucocorticoid that is selected from among cortisones, dexamethasones, hydrocortisones, methylprednisolones, prednisolones and prednisones.

### Chondroitinases

Chondroitinases are enzymes found throughout the animal kingdom which degrade glycosaminoglycans, specifically chondroitins and chondroitin sulfates, through an endoglycosidase reaction. In some embodiments the chondroitinase is a mammalian chondroitinase. In some embodiments the chondroitinase is a recombinant human chondroitinase. In some embodiments the chondroitinase is HYAL4. Other exemplary chondroitinases include chondroitinase ABC (derived from Proteus vulgaris; Japanese Patent Application Laid-open No 6-153947, T. Yamagata et al. J. Biol. Chem., 243, 1523 (1968), S. Suzuki et al, J. Biol. Chem., 243, 1543 (1968)), chondroitinase AC (derived from Flavobacterium heparinum; T. Yamagata et al., J. Biol. Chem., 243, 1523 (1968)), chondroitinase AC II (derived from Arthrobacter aurescens; K. Hiyama. and S. Okada, J. Biol. Chem., 250, 1824 (1975). K. Hiyama and S. Okada, J. Biochem. (Tokyo), 80, 1201 (1976)), Hyaluronidase ACIII (derived from Flavobacterium sp. Hp102; Hirofumi Miyazono et al., Seikagaku, 61, 1023 (1989)), chondroitinase B (derived from Flavobacterium heparinum: Y. M. Michelacci and C. P. Dietrich, Biochem. Biophys. Res. Commun., 56.973 (1974), Y. M. Michelacci and C. P. Dietrich, Biochem. J., 151, 121 (1975). Kenichi Maeyama et al, Seikagaku, 57, 1189 (1985)), chondroitinase C (derived from Flavobacterium sp. Hp102: Hirofumi Miyazono et al. Seikagaku, 61, 1023 (1939)), and the like.

### Matrix Metalloproteinases

Matrix metalloproteases (MMPs) are zinc-dependent endopeptidases that are the major proteases involved in extracellular matrix (ECM) degradation. MMPs are capable of degrading a wide range of extracellular molecules and a number of bioactive molecules. Twenty-four MMP genes have been identified in humans, which can be organized into six groups based on domain organization and substrate preference: Collagenases (MMP-1, -8 and -13). Gelatinases (MMP-2 and MMP-9). Stromelysins (MMP-3, -10 and -11), Matrilysin (MMP-7 and MMP-26). Membrane-type (MT)-MMPs (MMP-14, -15, -16, -17, -24 and -25) and others (MMP-12, -19, -20, -21, -23, -27 and -28). In some embodiments, the stromal modifying moiety is a human recombinant MMP (e.g., MMP-1, -2, -3, -4, -5, -6, -7, -8, -9, 10, -11, -12, -13, -14, 15, -15, -17, -18, -19, 20, -21, -22, -23, or -24).

### Collagenases

The three mammalian collagenases (MMP-1, -8, and -13) are the principal secreted endopeptidases capable of cleaving collagenous extracellular matrix. In addition to fibrillar collagens, collagenases can cleave several other matrix and non-matrix proteins including growth factors. Collagenases are synthesized as inactive pro-forms, and once activated, their activity is inhibited by specific tissue inhibitors of metalloproteinases, TIMPs, as well as by non-specific proteinase inhibitors (Ala-aho R et al. Biochimie. Collagenases in cancer. 2005 March-April; 87(3-4):273-86). In some embodiments, the stromal modifying moiety is a collagenase. In some embodiments, the collagenase is a human recombinant collagenase. In some embodiments, the collagenase is MMP-1. In some embodiments, the collagenase is MMP-8. In some embodiments, the collagenase is MMP-13.

### Macrophage Metalloelastase

Macrophage metalloelastase (MME), also known as MMP-12, is a member of the stromelysin subgroup of MMPs and catalyzes the hydrolysis of soluble and insoluble elastin and a broad selection of matrix and nonmatrix substrates including type IV collagen, fibronectin, laminin, vitronectin, entactin, heparan, and chondroitin sulfates (Erja Kerkelä et al. Journal of Investigative Dermatology (2000) 114, 1113-1119. doi:10.1046/j.1523-1747.2000.00993). In some embodiments, the stromal modifying moiety is a MME. In some embodiments, the MME is a human recombinant MME. In some embodiments, the MME is MMP-12.

### Additional Stromal Modifying Moieties

In some embodiments, the stromal modifying moiety causes one or more of: decreases the level or production of a stromal or extracellular matrix (ECM) component; decreases tumor fibrosis; increases interstitial tumor transport; improves tumor perfusion; expands the tumor microvasculature; decreases interstitial fluid pressure (IFP) in a tumor; or decreases or enhances penetration or diffusion of an agent, e.g., a cancer therapeutic or a cellular therapy, into a tumor or tumor vasculature.

In some embodiments, the stromal or ECM component decreased is chosen from a glycosaminoglycan or an extracellular protein, or a combination thereof. In some embodiments, the glycosaminoglycan is chosen from hyaluronan (also known as hyaluronic acid or HA), chondroitin sulfate, chondroitin, dermatan sulfate, heparin, heparin sulfate, entactin, tenascin, aggrecan and keratin sulfate. In some embodiments, the extracellular protein is chosen from collagen, laminin, elastin, fibrinogen, fibronectin, or vitronectin. In some embodiments, the stromal modifying moiety includes an enzyme molecule that degrades a tumor stroma or extracellular matrix (ECM). In some embodiments, the enzyme molecule is chosen from a hyaluronidase molecule, a collagenase molecule, a chondroitinase molecule, a matrix metalloproteinase molecule (e.g., macrophage metalloelastase), or a variant (e.g., a fragment) of any of the aforesaid. The term “enzyme molecule” includes a full length, a fragment or a variant of the enzyme, e.g., an enzyme variant that retains at least one functional property of the naturally-occurring enzyme.

In some embodiments, the stromal modifying moiety decreases the level or production of hyaluronic acid. In other embodiments, the stromal modifying moiety comprises a hyaluronan degrading enzyme, an agent that inhibits hyaluronan synthesis, or an antibody molecule against hyaluronic acid.

In some embodiments, the hyaluronan degrading enzyme is a hyaluronidase molecule, e.g., a full length or a variant (e.g., fragment thereof) thereof. In some embodiments, the hyaluronan degrading enzyme is active in neutral or acidic pH, e.g., pH of about 4-5. In some embodiments, the hyaluronidase molecule is a mammalian hyaluronidase molecule, e.g., a recombinant human hyaluronidase molecule. e.g., a full length or a variant (e.g., fragment thereof, e.g., a truncated form) thereof. In some embodiments, the hyaluronidase molecule is chosen from HYAL1. HYAL2, or PH-20/SPAM1, or a variant thereof (e.g., a truncated form thereof). In some embodiments, the truncated form lacks a C-terminal glycosylphosphatidylinositol (GPI) attachment site or a portion of the GPI attachment site. In some embodiments, the hyaluronidase molecule is glycosylated, e.g., comprises at least one N-linked glycan.

In some embodiments, the hyaluronidase molecule comprises the amino acid sequence: LNFRAPPVIPNVPFLWAWNAPSEFCLGKFDEPLDMSLFSFIGSPRINATGQGVTIFYVDRLGYYPYI DSITGVTVNGGIPQKISLQDHLDKAKKDITFYMPVDNLGMAVIDWEEWRPTWARNWKPKDVYK NRSIELVQQQNVQLSLTEATEKAKQEFEKAGKDFLVETIKLGKLLRPNHLWGYYLFPDCYNHHY KKPGYNGSCFNVEIKRNDDLSWLWNESTALYPSIYLNTQQSPVAATLYVRNRVREAIRVSKIPDA KSPLPVFAYTRIVFTDQVLKFLSQDELVYTFGETVALGASGIVIWGTLSIMRSMKSCLLLDNYME TILNPYIINVTLAAKMCSQVLCQEQGVCIRKNWNSSDYLHLNPDNFAIQLEKGGKFTVRGKPTLE DLEQFSEKFYCSCYSTLSCKEKADVKDTDAVDVCIADGVCIDAFLKPPMETEEPQIFYNASPSTLS (SEQ ID NO:3311), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3311.

In some embodiments, the hyaluronidase molecule comprises: (i) the amino acid sequence of 36-464 of SEQ ID NO: 3311; (ii) the amino acid sequence of 36-481, 36482, or 36-483 of PH20, wherein PH20 has the sequence of amino acids set forth in SEQ ID NO: 3311; or (iii) an amino acid sequence having at least 95% to 100% sequence identity to the polypeptide or truncated form of sequence of amino acids set forth in SEQ ID NO: 3311; or (iv) an amino acid sequence having 30, 20, 10, 5 or fewer amino acid substitutions to the amino acid sequence set forth in SEQ ID NO: 3311. In some embodiments, the hyaluronidase molecule comprises an amino acid sequence at least 95% (e.g., at least 95%, 96%, 97%, 98%, 99%, 100%) identical to the amino acid sequence of SEQ ID NO: 3311. In some embodiments, the hyaluronidase molecule is encoded by a nucleotide sequence at least 95% (e.g., at least 96%, 97%, 98%, 99%, 100%) identical to the nucleotide sequence of SEQ ID NO: 3311.

In some embodiments, the hyaluronidase molecule is PH20, e.g., rHuPH20. In some embodiments, the hyaluronidase molecule is HYAL1 and comprises the amino acid sequence: FRGPLLPNRPFTTVWNANTQWCLERHGVDVDVSVFDVVANPGQTFRGPDMTIFYSSQGTYPYYT PTGEPVFGGLPQNASLIAHLARTFQDILAAIPAPDFSGLAVIDWEAWRPRWAFNWDTKDIYRQRS RALVQAQHPDWPAPQVEAVAQDQFQGAARAWMAGTLQLGRALRPRGLWGFYGFPDCYNYDF LSPNYTGQCPSGIRAQNDQLGWLWGQSRALYPSIYMPAVLEGTGKSQMYVQHRVAEAFRVAVA AGDPNLPVLPYVQIFYDTTNHFLPLDELEHSLGESAAQGAAGVVLWVSWENTRTKESCQAIKEY MDTTLGPFILNVTSGALLCSQALCSGHGRCVRRTSHPKALLLLNPASFSIQLTPGGGPLSLRGALS LEDQAQMAVEFKCRCYPGWQAPWCERKSMW (SEQ ID NO: 3312), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3312.

In some embodiments, the hyaluronan degrading enzyme, e.g., the hyaluronidase molecule, further comprises a polymer, e.g., is conjugated to a polymer, e.g., PEG. In some embodiments, the hyaluronan-degrading enzyme is a PEGylated PH20 enzyme (PEGPH20). In some embodiments, the hyaluronan degrading enzyme, e.g., the hyaluronidase molecule, further comprises an immunoglobulin chain constant region (e.g., Fc region) chosen from, e.g., the heavy chain constant regions of IgG1, IgG2, IgG3, and IgG4, more particularly, the heavy chain constant region of human IgG1, IgG2, IgG3, or IgG4. In some embodiments, the immunoglobulin constant region (e.g., the Fc region) is linked, e.g., covalently linked to, the hyaluronan degrading enzyme, e.g., the hyaluronidase molecule. In some embodiments, the immunoglobulin chain constant region (e.g., Fc region) is altered, e.g., mutated, to increase or decrease one or more of: Fc receptor binding, antibody glycosylation, the number of cysteine residues, effector cell function, or complement function. In some embodiments, the hyaluronan degrading enzyme, e.g., the hyaluronidase molecule forms a dimer.

In some embodiments, the stromal modifying moiety comprises an inhibitor of the synthesis of hyaluronan, e.g., an HA synthase. In some embodiments, the inhibitor comprises a sense or an antisense nucleic acid molecule against an HA synthase or is a small molecule drug. In some embodiments, the inhibitor is 4-methylumbelliferone (MU) or a derivative thereof (e.g., 6,7-dihydroxy-4-methyl coumarin or 5,7-dihydroxy-4-methyl coumarin), or leflunomide or a derivative thereof.

In some embodiments, the stromal modifying moiety comprises antibody molecule against hyaluronic acid.

In some embodiments, the stromal modifying moiety comprises a collagenase molecule, e.g., a mammalian collagenase molecule, or a variant (e.g., fragment) thereof. In some embodiments, the collagenase molecule is collagenase molecule IV, e.g., comprising the amino acid sequence of: YNFFPRKPKWDKNQITYRIIGYTPDLDPETVDDAFARAFQVWSDVTPLRFSRIHDGEADIMINFGR WEHGDGYPFDGKDGLLAHAFAPGTGVGGDSHFDDDELWTLGEGQVVRVKYGNADGEYCKFPF LFNGKEYNSCTDTGRSDGFLWCSTTYNFEKDGKYGFCPHEALFTMGGNAEGQPCKFPFRFQGTS YDSCTTEGRTDGYRWCGTTEDYDRDKKYGFCPETAMSTVGGNSEGAPCVFPFTFLGNKYESCTS AGRSDGKMWCATTANYDDDRKWGFCPDQGYSLFLVAAHEFGHAMGLEHSQDPGALMAPIYTY TKNFRLSQDDIKGIQELYGASPDIDLGTGPTPTLGPVTPEICKQDIVFDGIAQIRGEIFFFKDRFIWR TVTPRDKPMGPLLVATFWPELPEKIDAVYEAPQEEKAVFFAGNEYWIYSASTLERGYPKPLTSLG LPPDVQRVDAAFNWSKNKKTYIFAGDKFWRYNEVKKKMDPGFPKLIADAWNAIPDNLDAVVD LQGGGHSYFFKGAYYLKLENQSLKSVKFGSIKSDWLGC (SEQ ID NO: 3313), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3313.

### Tumor Antigen Moiety

In some embodiments, the multifunctional molecule further includes a tumor antigen moiety. In some embodiments, the tumor-targeting moiety is an antigen, e.g., a cancer antigen. In some embodiments, the cancer antigen is a tumor antigen or stromal antigen, or a hematological antigen.

“Cancer” as used herein can encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

In some embodiments, the tumor-targeting moiety, e.g., cancer antigen, is chosen from: BCMA, FcRH5, CD19, CD20, CD22, CD30, CD33, CD38, CD47, CD99, CD123, FcRH5, CLEC12, CD179A, SLAMF7, or NY-ESO1, PDL1, CD47, gangloside 2 (GD2), prostate stem cell antigen (PSCA), prostate specific membrane antigen (PMSA), prostate-specific antigen (PSA), carcinoembryonic antigen (CEA). Ron Kinase, c-Met, Immature laminin receptor. TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17. Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, α actinin-4, TRP1/gp75, TRP2, gp100, Melan-A/MART1, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, gpA33, GD3, GM2, VEGFR, Intergrins (Integrin alphaVbeta3, Integrin alpha5Beta1), Carbohydrates (Le). IGF1R, EPHA3, TRAILR1, TRAILR2, RANKL, (FAP), TGF-beta, hyaluronic acid, collagen, e.g., collagen IV, tenascin C, or tenascin W. In some embodiments, the tumor-targeting moiety, e.g., cancer antigen, is BCMA. In some embodiments, the tumor-targeting moiety, e.g., cancer antigen, is FcRH5.

In some embodiments, the tumor-targeting moiety, e.g., cancer antigen, is chosen from: CD19. CD123, CD22, CD30, CD171, CS-1, C-type lectin-like molecule-1, CD33, epidermal growth factor receptor variant III (EGFRvIII), ganglioside G2 (GD2), ganglioside GD3, TNF receptor family member B cell maturation (BCMA), Tn antigen ((Tn Ag) or (GalNAcα-Ser/Thr)), prostate-specific membrane antigen (PSMA). Receptor tyrosine kinase-like orphan receptor 1 (ROR1). Fms-Like Tyrosine Kinase 3 (FLT3). Tumor-associated glycoprotein 72 (TAG72), CD38, CD44v6, Carcinoembryonic antigen (CEA). Epithelial cell adhesion molecule (EPCAM), B7H3 (CD276), KIT (CD117), Interleukin-13 receptor subunit alpha-2, mesothelin. Interleukin 11 receptor alpha (IL-11Ra), prostate stem cell antigen (PSCA), Protease Serine 21, vascular endothelial growth factor receptor 2 (VEGFR2). Lewis(Y) antigen, CD24. Platelet-derived growth factor receptor beta (PDGFR-beta). Stage-specific embryonic antigen-4 (SSEA-4), CD20, Folate receptor alpha, Receptor tyrosine-protein kinase ERBB2 (Her2/neu). Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type, 9 (LMP2), glycoprotein 100 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2). Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRCSD), chromosome X open reading frame 61 (CXORF61), CD97, CD179a, anaplastic lymphoma kinase (ALK), Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 complex, locus K 9 (LY6K), Olfactory receptor 51E2 (ORS1E2), TCR Gamma Alternate Reading Frame Protein (TARP), Wilms tumor protein (WT1), Cancer/testis antigen 1 (NY-ESO-1), Cancer/testis antigen 2 (LAGE-1a), Melanoma-associated antigen 1 (MAGE-A1), ETS translocation-variant gene 6, located on chromosome 2p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2). Fos-related antigen 1, tumor protein p53 (p53), p53 mutant, prostein, surviving, telomerase, prostate carcinoma tumor antigen-1, melanoma antigen recognized by T cells 1. Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene), N-Acetyl glucosaminyl-transferase V (NA17), paired box protein Pax-3 (PAX3). Androgen receptor, Cyclin B1, v-myc avian myelocytomatosis viral oncogene neuroblastoma derived homolog (MYCN), Ras Homolog Family Member C (RhoC). Tyrosinase-related protein 2 (TRP-2). Cytochrome P450 1B1 (CYP1B1), CCCTC-Binding Factor (Zinc Finger Protein)-Like. Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK). A kinase anchor protein 4 (AKAP-4), synovial sarcoma, X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated inmunoglobulin-like receptor 1 (LAIR1), Fc fragment of IgA receptor (FCAR or CD89), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), or immunoglobulin lambda-like polypeptide 1 (IGLL1).

### FcRH5 Targeting Moieties:

In some embodiments, the multispecific molecules as described herein include a targeting moiety that binds to FcRH5 (e.g., a FcRH5 targeting moiety). The FcRH5 targeting moiety can be chosen from an antibody molecule (e.g., an antigen binding domain as described herein), a receptor or a receptor fragment, or a ligand or a ligand fragment, or a combination thereof. In some embodiments, the FcRH5 targeting moiety associates with, e.g., binds to, a cancer or hematopoietic cell (e.g., a molecule, e.g., antigen, present on the surface of the cancer or hematopoietic cell). In certain embodiments, the FcRH5 targeting moiety targets, e.g., directs the multispecific molecules as described herein to a cancer or hematopoietic cell. In some embodiments, the cancer is a hematological cancer, e.g., multiple myeloma.

In some embodiments, the multispecific molecule, e.g., the FcRH5 targeting moiety, binds to a FcRH5 antigen on the surface of a cell, e.g., a cancer or hematopoietic cell. The FcRH5 antigen can be present on a primary tumor cell, or a metastatic lesion thereof. In some embodiments, the cancer is a hematological cancer, e.g., multiple myeloma. For example, the FcRH5 antigen can be present on a tumor. e.g., a tumor of a class typified by having one or more of: limited tumor perfusion, compressed blood vessels, or fibrotic tumor interstitium.

The multispecific molecules described herein includes a FcRH5 targeting moiety that comprises an anti-FcRH5 antibody or antigen-binding fragment thereof described in U.S. Pat. No. 7,999,077. US20150098900, U.S. Pat. Nos. 8,299,220, 7,105,149, 8,362,213, 8,466,260, 8,617,559, US20160368985. US20150166661, and US20080247944, the entire contents of any of the aforesaid publications are herein incorporated by reference.

In some embodiments, the multispecific molecules described herein includes a FcRH5 targeting moiety that comprises an anti-FcRH5 antibody or antigen-binding fragment thereof described in U.S. Pat. No. 7,999,077, the entire contents of which are herein incorporated by reference.

### BCMA Targeting Moieties:

In certain embodiments, the multispecific molecules as described herein include a targeting moiety that binds to BCMA (e.g., a BCMA targeting moiety). The BCMA targeting moiety can be chosen from an antibody molecule (e.g., an antigen binding domain as described herein), a receptor or a receptor fragment, or a ligand or a ligand fragment, or a combination thereof. In some embodiments, the BCMA targeting moiety associates with, e.g., binds to, a cancer or hematopoietic cell (e.g., a molecule, e.g., antigen, present on the surface of the cancer or hematopoietic cell). In certain embodiments, the BCMA targeting moiety targets, e.g., directs the multispecific molecules as described herein to a cancer or hematopoietic cell. In some embodiments, the cancer is a hematological cancer, e.g., multiple myeloma.

In some embodiments, the multispecific molecule, e.g., the BCMA targeting moiety, binds to a BCMA antigen on the surface of a cell, e.g., a cancer or hematopoietic cell. The BCMA antigen can be present on a primary tumor cell, or a metastatic lesion thereof. In some embodiments, the cancer is a hematological cancer, e.g., multiple myeloma. For example, the BCMA antigen can be present on a tumor, e.g., a tumor of a class typified by having one or more of limited tumor perfusion, compressed blood vessels, or fibrotic tumor interstitium.

Exemplary BCMA targeting moieties: the multispecific molecules described herein can include a BCMA targeting moiety that comprises an anti-BCMA antibody or antigen-binding fragment thereof described in U.S. Pat. Nos. 8,920,776, 9,243,058, 9,340,621, 8,846,042, 7,083,785, 9,545,086, 7,276,241, 9,034,324, 7,799,902, 9,387,237, 8,821,883, 8,617,45, US20130273055, US20160176973, US20150368351, US20150376287, US20170022284, US20160015749, US20140242077, US20170037128, US20170051068, US20160368988, US20160311915, US20160131654, US20120213768, US20110177093, US20160297885, EP3137500, EP2699259, EP2982694, EP3029068, EP3023437, WO2016090327, WO2017021450, WO2016110584, WO2016118641, WO2016168149, the entire contents of which are incorporated herein by reference.

In some embodiments, the BCMA-targeting moiety includes an antibody molecule (e.g., Fab or scFv) that binds to BCMA. In some embodiments, the antibody molecule to BCMA comprises one, two, or three CDRs from any of the heavy chain variable domain sequences of Table 1, or a closely related CDR, e.g., CDRs which have at least one amino acid alteration, but not more than two, three or four alterations (e.g., substitutions, deletions, or insertions, e.g., conservative substitutions) from any of the CDR sequences of Table 15. In some embodiments, the antibody molecule to BCMA comprises a heavy chain variable domain sequence chosen from any of the amino acid sequences of Table 15, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)).

Alternatively, or in combination with the heavy chain to BCMA as described herein, the antibody molecule to BCMA comprises one, two, or three CDRs from any of the light chain variable domain sequences of Table 15, or a closely related CDR, e.g., CDRs which have at least one amino acid alteration, but not more than two, three or four alterations (e.g., substitutions, deletions, or insertions, e.g., conservative substitutions) from any of the CDR sequences of Table 15. In some embodiments, the antibody molecule to BCMA comprises a light chain variable domain sequence chosen from any of the amino acid sequences of Table 15, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)).

### Tumor-Targeting Moieties

In some embodiments, the multifunctional or multispecific (e.g., bi-, tri-, tetra-specific) molecules as described herein further include, e.g., are engineered to further contain, one or more tumor specific targeting moieties that direct the molecule to a tumor cell.

In certain embodiments, the multispecific molecules as described herein further include a tumor-targeting moiety. The tumor targeting moiety can be chosen from an antibody molecule (e.g., an antigen binding domain as described herein), a receptor or a receptor fragment, or a ligand or a ligand fragment, or a combination thereof. In some embodiments, the tumor targeting moiety associates with, e.g., binds to, a tumor cell (e.g., a molecule, e.g., antigen, present on the surface of the tumor cell). In certain embodiments, the tumor targeting moiety targets, e.g., directs the multispecific molecules as described herein to a cancer (e.g., a cancer or tumor cells). In some embodiments, the cancer is chosen from a hematological cancer, a solid cancer, a metastatic cancer, or a combination thereof.

In some embodiments, the multispecific molecule, e.g., the tumor-targeting moiety, binds to a solid tumor antigen or a stromal antigen. The solid tumor antigen or stromal antigen can be present on a solid tumor, or a metastatic lesion thereof. In some embodiments, the solid tumor is chosen from one or more of pancreatic (e.g., pancreatic adenocarcinoma), breast, colorectal, lung (e.g., small or non-small cell lung cancer), skin, ovarian, or liver cancer. In some embodiments, the solid tumor is a fibrotic or desmoplastic solid tumor. For example, the solid tumor antigen or stromal antigen can be present on a tumor, e.g., a tumor of a class typified by having one or more of: limited tumor perfusion, compressed blood vessels, or fibrotic tumor interstitium.

In certain embodiments, the solid tumor antigen is chosen from one or more of: PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, CD47, α actinin-4, TRP1/gp75, TRP2, gp100, Melan-A/MART1, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, EGFRvIII, gpA33, GD3, GM2, VEGFR, Intergrins (Integrin alphaVbeta3, Integrin alpha5Beta1), Carbohydrates (Le), IGF1R, EPHA3, TRAILR1, TRAILR2, or RANKL.

In other embodiments, the multispecific molecule, e.g., the tumor-targeting moiety, binds to a molecule, e.g., antigen, present on the surface of a hematological cancer, e.g., a leukemia or a lymphoma. In some embodiments, the hematological cancer is a B-cell or T cell malignancy. In some embodiments, the hematological cancer is chosen from one or more of a Hodgkin's lymphoma, Non-Hodgkin's lymphoma (e.g., B cell lymphoma, diffuse large B cell lymphoma, follicular lymphoma, chronic lymphocytic leukemia, mantle cell lymphoma, marginal zone B-cell lymphoma, Burkitt lymphoma, lymphoplasmacytic lymphoma, hairy cell leukemia), acute myeloid leukemia (AML), chronic myeloid leukemia, myelodysplastic syndrome (MDS), multiple myeloma, or acute lymphocytic leukemia. In embodiments, the cancer is other than acute myeloid leukemia (AML) or myelodysplastic syndrome (MDS). In embodiments, the hematological antigen is chosen from CD47, CD99, CD30, CD38, SLAMF7, or NY-ESO1. In some embodiments, the hematological antigen is chosen from is chosen from one or more of: BCMA, CD19, CD20, CD22, CD33, CD123, FcRH5, CLEC12, or CD179A.

Antibody Molecules 1004931In some embodiments, the antibody molecule binds to a cancer antigen, e.g., a tumor antigen or a stromal antigen. In some embodiments, the cancer antigen is, e.g., a mammalian, e.g., a human, cancer antigen. In other embodiments, the antibody molecule binds to an immune cell antigen, e.g., a mammalian, e.g., a human, immune cell antigen. For example, the antibody molecule binds specifically to an epitope, e.g., linear or conformational epitope, on the cancer antigen or the immune cell antigen.

In some embodiments, an antibody molecule is a monospecific antibody molecule and binds a single epitope. E.g., a monospecific antibody molecule having a plurality of immunoglobulin variable domain sequences, each of which binds the same epitope.

In some embodiments, an antibody molecule is a multispecific or multifunctional antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a multispecific antibody molecule comprises a third, fourth or fifth immunoglobulin variable domain. In some embodiments, a multispecific antibody molecule is a bispecific antibody molecule, a trispecific antibody molecule, or a tetraspecific antibody molecule.

In some embodiments, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some embodiments, a bispecific antibody molecule comprises a half antibody having binding specificity for a first epitope and a half antibody having binding specificity for a second epitope. In some embodiments, a bispecific antibody molecule comprises a half antibody, or fragment thereof, having binding specificity for a first epitope and a half antibody, or fragment thereof, having binding specificity for a second epitope. In some embodiments, a bispecific antibody molecule comprises a scFv or a Fab, or fragment thereof, have binding specificity for a first epitope and a scFv or a Fab, or fragment thereof, have binding specificity for a second epitope.

in some embodiments, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In some embodiments, an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)2. Fc, Fd, Fd′. Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG. IgA. IgM. IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody. (v) a diabody (dAb) fragment, which consists of a VH domain; (vi) a camelid or camelized variable domain; (vii) a single chain Fv, (scFv), see e.g., Bird et al. (1988) Science 242:423-426, and Huston et al. (1988) Proc. Natl. Acad Sci. USA 85:5879-5883); (viii) a single domain antibody. These antibody fragments are obtained using conventional techniques known to those with skill in the art, and the fragments are screened for utility in the same manner as are intact antibodies.

Antibody molecules include intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see. Kabat. E. A., et al. (1991) Sequences of Proteins of Immunological Interest, Fifth Edition. U.S. Department of Health and Human Services, NIH Publication No. 91-3242: Chothia. C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda. MD (“Kabat” numbering scheme). Al-Lazikani et al., (1997) JMB 273.927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2. FR3, CDR3, FR4.

The antibody molecule can be a polyclonal or a monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody can be recombinantly produced, e.g., produced by phage display or by combinatorial methods, or by yeast display.

Phage display and combinatorial methods for generating antibodies are known in the art (as described in, e.g., Ladner et al, U.S. Pat. No. 5,223,409; Kang el al. International Publication No. WO 92/18619: Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibody Hybridomas 3:81-85: Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:41334137; and Barbas et al. (1991) PNAS 88:7978-7982, the contents of all of which are incorporated by reference herein).

The yeast display method for generating or identifying antibodies is known in the art, e.g., as described in Chao et al. (2006) Nature Protocols 1(2):755-68, the entire contents of which is incorporated by reference herein.

In some embodiments, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741: Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Nat. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody molecule can be one in which the variable region, or a portion thereof, e.g., the CDRs, are generated in a non-human organism, e.g., a rat or mouse. Chimeric, CDR-grafted, and humanized antibodies are within the invention. Antibody molecules generated in a non-human organism. e.g., a rat or mouse, and then modified, e.g., in the variable framework or constant region, to decrease antigenicity in a human are within the invention.

An “effectively human” protein is a protein that does substantially not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi. M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding to the antigen. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In some embodiments, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft. Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody molecule can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibody molecules can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibody molecules in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann NY Acad Sci 880:263-80; and Reiter. Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In some embodiments the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company. Rockford. Ill.

### CDR-Grafted Scaffolds

In some embodiments, the antibody molecule is a CDR-grafted scaffold domain. In some embodiments, the scaffold domain is based on a fibronectin domain, e.g., fibronectin type Ill domain. The overall fold of the fibronectin type III (Fn3) domain is closely related to that of the smallest functional antibody fragment, the variable domain of the antibody heavy chain. There are three loops at the end of Fn3; the positions of BC, DE and FG loops approximately correspond to those of CDR1, 2 and 3 of the VH domain of an antibody. Fn3 does not have disulfide bonds; and therefore Fn3 is stable under reducing conditions, unlike antibodies and their fragments (see, e.g., WO 98/56915; WO 01/64942; WO 00/34784). An Fn3 domain can be modified (e.g., using CDRs or hypervariable loops described herein) or varied, e.g., to select domains that bind to an antigen/marker/cell described herein.

In some embodiments, a scaffold domain, e.g., a folded domain, is based on an antibody, e.g., a “minibody” scaffold created by deleting three beta strands from a heavy chain variable domain of a monoclonal antibody (see, e.g., Tramontano et al., 1994, J Mol. Recognit. 7:9; and Martin et al., 1994. EMBO J. 13:5303-5309). The “minibody” can be used to present two hypervariable loops. In some embodiments, the scaffold domain is a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess, 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070).

Other exemplary scaffold domains include but are not limited to T-cell receptors; MHC proteins; extracellular domains (e.g., fibronectin Type III repeats. EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains). See, e.g., US 20040009530 and U.S. Pat. No. 7,501,121, incorporated herein by reference.

In some embodiments, a scaffold domain is evaluated and chosen, e.g., by one or more of the following criteria: (1) amino acid sequence. (2) sequences of several homologous domains, (3) 3-dimensional structure, and/or (4) stability data over a range of pH, temperature, salinity, organic solvent, oxidant concentration. In some embodiments, the scaffold domain is a small, stable protein domain, e.g., a protein of less than 100, 70, 50, 40 or 30 amino acids. The domain may include one or more disulfide bonds or may chelate a metal, e.g., zinc.

### Antibody-Based Fusions

A variety of formats can be generated which contain additional binding entities attached to the N or C terminus of antibodies. These fusions with single chain or disulfide stabilized Fvs or Fabs result in the generation of tetravalent molecules with bivalent binding specificity for each antigen. Combinations of scFvs and scFabs with IgGs enable the production of molecules which can recognize three or more different antigens.

### Antibody-Fab Fusion

Antibody-Fab fusions are bispecific antibodies comprising a traditional antibody to a first target and a Fab to a second target fused to the C terminus of the antibody heavy chain. Commonly the antibody and the Fab will have a common light chain. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma. J. et al. (1997) Nature Biotech 15:159.

### Antibody-scFv Fusion

Antibody-scFv Fusions are bispecific antibodies comprising a traditional antibody and a scFv of unique specificity fused to the C terminus of the antibody heavy chain. The scFv can be fused to the C terminus through the Heavy Chain of the scFv either directly or through a linker peptide. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Variable Domain Immunoglobulin DVD

A related format is the dual variable domain immunoglobulin (DVD), which are composed of VH and VL domains of a second specificity place upon the N termini of the V domains by shorter linker sequences.

Other exemplary multispecific antibody formats include, e.g., those described in the following US20160114057A1, US20130243775A1, US20140051833, US20130022601, US20150017187A1, US20120201746A1, US20150133638A1, US20130266568A1, US20160145340A1, WO2015127158A1. US20150203591A1, US20140322221A1, US20130303396A1, US20110293613, US20130017200A1. US20160102135A1, WO2015197598A2, WO2015197582A1, U.S. Pat. No. 9,359,437, US20150018529, WO2016115274A1, WO2016087416A1, US20080069820A1, U.S. Pat. Nos. 9,145,588B, 7,919,257, and US20150232560A1. Exemplary multispecific molecules utilizing a full antibody-Fab/scFab format include those described in the following, U.S. Pat. No. 9,382,323B2, US20140072581A 1, US20140308285A1, US20130165638A1, US20130267686A1, US20140377269A1, U.S. Pat. No. 7,741,446B2, and WO1995009917A1. Exemplary multispecific molecules utilizing a domain exchange format include those described in the following, US20150315296A1, WO2016087650A1, US20160075785A1, WO2016016299A1, US20160130347A1, US20150166670, U.S. Pat. No. 8,703,132B2, US20100316645, U.S. Pat. No. 8,227,577B2, US20130078249.

### Fc-Containing Multispecific Molecules

In some embodiments, the multispecific molecules as described herein includes an immunoglobulin constant region (e.g., an Fc region). Exemplary Fc regions can be chosen from the heavy chain constant regions of IgG1, IgG2, IgG3 or IgG4; more particularly, the heavy chain constant region of human IgG1, IgG2, IgG3, or IgG4.

In some embodiments, the immunoglobulin chain constant region (e.g., the Fc region) is altered, e.g., mutated, to increase or decrease one or more of: Fc receptor binding, antibody glycosylation, the number of cysteine residues, effector cell function, or complement function.

In other embodiments, an interface of a first and second immunoglobulin chain constant regions (e.g., a first and a second Fc region) is altered, e.g., mutated, to increase or decrease dimerization, e.g., relative to a non-engineered interface, e.g., a naturally-occurring interface. For example, dimerization of the immunoglobulin chain constant region (e.g., the Fc region) can be enhanced by providing an Fc interface of a first and a second Fc region with one or more of: a paired protuberance-cavity (“knob-in-a hole”), an electrostatic interaction, or a strand-exchange, such that a greater ratio of heteromultimer to homomultimer forms, e.g., relative to a non-engineered interface.

In some embodiments, the multispecific molecules include a paired amino acid substitution at a position chosen from one or more of 347, 349, 350, 351, 366, 368, 370, 392, 394, 395, 397, 398, 399, 405, 407, or 409, e.g., of the Fc region of human IgG1 For example, the immunoglobulin chain constant region (e.g., Fc region) can include a paired an amino acid substitution chosen from: T366S, L368A, or Y407V (e.g., corresponding to a cavity or hole), and T366W (e.g., corresponding to a protuberance or knob).

In other embodiments, the multifunctional molecule includes a half-life extender, e.g., a human serum albumin or an antibody molecule to human serum albumin.

In some embodiments, Fc contains exemplary Fc modifications listed in Table 14.

### Heterodimerized Antibody Molecules & Methods of Making

Various methods of producing multispecific antibodies have been disclosed to address the problem of incorrect heavy chain pairing. Exemplary methods are described below. Exemplary multispecific antibody formats and methods of making said multispecific antibodies are also disclosed in e.g., Speiss et al. Molecular Immunology 67 (2015) 95-106; and Klein et al mAbs 4:6, 653-663; November/December 2012: the entire contents of each of which are incorporated by reference herein.

Heterodimerized bispecific antibodies are based on the natural IgG structure, wherein the two binding arms recognize different antigens, IgG derived formats that enable defined monovalent (and simultaneous) antigen binding are generated by forced heavy chain heterodimerization, combined with technologies that minimize light chain mispairing (e.g., common light chain). Forced heavy chain heterodimerization can be obtained using, e.g., knob-in-hole OR strand exchange engineered domains (SEED).

### Knob-in-Hole

Knob-in-Hole as described in U.S. Pat. Nos. 5,731,116, 7,476,724 and Ridgway, J. et al. (1996) Prot. Engineering 9(7): 617-621, broadly involves: (1) mutating the CH3 domain of one or both antibodies to promote heterodimerization, and (2) combining the mutated antibodies under conditions that promote heterodimerization. “Knobs” or “protuberances” are typically created by replacing a small amino acid in a parental antibody with a larger amino acid (e.g., T366Y or T366W); “Holes” or “cavities” are created by replacing a larger residue in a parental antibody with a smaller amino acid (e.g., Y407T, T366S, L368A and/or Y407V).

For bispecific antibodies including an Fc domain, introduction of specific mutations into the constant region of the heavy chains to promote the correct heterodimerization of the Fc portion can be utilized. Several such techniques are reviewed in Klein et al. (mAbs (2012) 4:6, 1-11), the contents of which are incorporated herein by reference in their entirety. These techniques include the “knobs-into-holes” (KiH) approach which involves the introduction of a bulky residue into one of the CH3 domains of one of the antibody heavy chains. This bulky residue fits into a complementary “hole” in the other CH3 domain of the paired heavy chain so as to promote correct pairing of heavy chains (see e.g., U.S. Pat. No. 7,642,228).

Exemplary KiH mutations include S354C, T366W in the “knob” heavy chain and Y349C, T366S, L368A, Y407V in the “hole” heavy chain. Other exemplary KiH mutations are provided in Table 4, with additional optional stabilizing Fc cysteine mutations.

Other Fc mutations are provided by Igawa and Tsunoda who identified 3 negatively charged residues in the CH3 domain of one chain that pair with three positively charged residues in the CH3 domain of the other chain. These specific charged residue pairs are: E356-K439. E357-K370. D399-K409 and vice versa. By introducing at least two of the following three mutations in chain A: E356K. E357K and D399K, as well as K370E. K409D, K439E in chain B, alone or in combination with newly identified disulfide bridges, they were able to favor very efficient heterodimerization while suppressing homodimerization at the same time (Martens T et al. A novel one-armed antic-Met antibody inhibits glioblastoma growth in vivo. Clin Cancer Res 2006; 12:6144-52; PMID:17062691). Xencor defined 41 variant pairs based on combining structural calculations and sequence information that were subsequently screened for maximal heterodimerization, defining the combination of S364H, F405A (HA) on chain A and Y349T, T394F on chain B (TF) (Moore G L et al. A novel bispecific antibody format enables simultaneous bivalent and monovalent co-engagement of distinct target antigens. MAbs 2011; 3:546-57; PMID: 22123055).

Other exemplary Fc mutations to promote heterodimerization of multispecific antibodies include those described in the following references, the contents of each of which is incorporated by reference herein, WO2016071377A1, US20140079689A1, US20160194389A1, US20160257763, WO2016071376A2, WO2015107026A1, WO2015107025A1, WO2015107015A1, US20150353636A1, US20140199294A1, U.S. Pat. No. 7,750,128B2, US20160229915A1, US20150344570A1, U.S. Pat. No. 8,003,774A1, US20150337049A1, US20150175707A1, US20140242075A1, US20130195849A1, US20120149876A1, US20140200331A1, U.S. Pat. No. 9,309,311B2, U.S. Pat. No. 8,586,713, US20140037621A1, US20130178605A1, US20140363426A1, US20140051835A1 and US20110054151A1.

Stabilizing cysteine mutations have also been used in combination with KiH and other Fc heterodimerization promoting variants, see e.g., U.S. Pat. No. 7,183,076. Other exemplary cysteine modifications include, e.g., those disclosed in US20140348839A1, U.S. Pat. No. 7,855,275B2, and U.S. Pat. No. 9,000,130B2.

### Strand Exchange Engineered Domains (SEED)

Heterodimeric Fc platform that support the design of bispecific and asymmetric fusion proteins by devising strand-exchange engineered domain (SEED) C(H)3 heterodimers are known. These derivatives of human IgG and IgA C(H)3 domains create complementary human SEED C(H)3 heterodimers that are composed of alternating segments of human IgA and IgG C(H)3 sequences. The resulting pair of SEED C(H)3 domains preferentially associates to form heterodimers when expressed in mammalian cells. SEEDbody (Sb) fusion proteins consist of [IgG1 hinge]-C(H)2-[SEED C(H)3], that may be genetically linked to one or more fusion partners (see e.g., Davis J H et al. SEED bodies: fusion proteins based on strand exchange engineered domain (SEED) CH3 heterodimers in an Fc analogue platform for asymmetric binders or immunofusions and bispecific antibodies. Protein Eng Des Se 2010: 23:195-202; PMID:20299542 and U.S. Pat. No. 8,871,912. The contents of each of which are incorporated by reference herein).

### Fc-Containing Entities (Mini-Antibodies)

Fc-containing entities, also known as mini-antibodies, can be generated by fusing scFv to the C-termini of constant heavy region domain 3 (CH3-scFv) and/or to the hinge region (scFv-hinge-Fc) of an antibody with a different specificity. Trivalent entities can also be made which have disulfide stabilized variable domains (without peptide linker) fused to the C-terminus of CH3 domains of IgGs.

### Duobody

“Duobody” technology to produce bispecific antibodies with correct heavy chain pairing are known. The DuoBody technology involves three basic steps to generate stable bispecific human IgG1 antibodies in a post-production exchange reaction. In a first step, two IgG1s, each containing single matched mutations in the third constant (CH3) domain, are produced separately using standard mammalian recombinant cell lines. Subsequently, these IgG1 antibodies are purified according to standard processes for recovery and purification. After production and purification (post-production), the two antibodies are recombined under tailored laboratory conditions resulting in a bispecific antibody product with a very high yield (typically >95%) (see e.g., Labrijn et al, PNAS 2013:110(13):5145-5150 and Labrijn et al. Nature Protocols 2014; 9(10):2450-63, the contents of each of which are incorporated by reference herein).

### Electrostatic Interactions

Methods of making multispecific antibodies using CH3 amino acid changes with charged amino acids such that homodimer formation is electrostatically unfavorable are disclosed. EP1870459 and WO 2009089004 describe other strategies for favoring heterodimer formation upon co-expression of different antibody domains in a host cell. In these methods, one or more residues that make up the heavy chain constant domain 3 (CH3), CH3-CH3 interfaces in both CH3 domains are replaced with a charged amino acid such that homodimer formation is electrostatically unfavorable and heterodimerization is electrostatically favorable. Additional methods of making multispecific molecules using electrostatic interactions are described in the following references, the contents of each of which is incorporated by reference herein, include US20100015133, U.S. Pat. No. 8,592,562B2, U.S. Pat. No. 9,200,060B2, US20140154254A1, and U.S. Pat. No. 9,358,286A1.

### Common Light Chain

Light chain mispairing needs to be avoided to generate homogenous preparations of bispecific IgGs. One way to achieve this is through the use of the common light chain principle, i.e. combining two binders that share one light chain but still have separate specificities. An exemplary method of enhancing the formation of a desired bispecific antibody from a mixture of monomers is by providing a common variable light chain to interact with each of the heteromeric variable heavy chain regions of the bispecific antibody. Compositions and methods of producing bispecific antibodies with a common light chain as disclosed in, e.g., U.S. Pat. No. 7,183,076B2, US20110177073A1, EP2847231A1, WO2016079081A1, and EP3055329A1, the contents of each of which is incorporated by reference herein.

### CrosMab

Another option to reduce light chain mispairing is the CrossMab technology which avoids non-specific L chain mispairing by exchanging CH1 and CL domains in the Fab of one half of the bispecific antibody. Such crossover variants retain binding specificity and affinity, but make the two arms so different that L chain mispairing is prevented. The CrossMab technology (as reviewed in Klein et al. Supra) involves domain swapping between heavy and light chains so as to promote the formation of the correct pairings. Briefly, to construct a bispecific IgG-like CrossMab antibody that could bind to two antigens by using two distinct light chain-heavy chain pairs, a two-step modification process is applied. First, a dimerization interface is engineered into the C-terminus of each heavy chain using a heterodimerization approach, e.g., Knob-into-hole (KiH) technology, to ensure that only a heterodimer of two distinct heavy chains from one antibody (e.g., Antibody A) and a second antibody (e.g., Antibody B) is efficiently formed. Next, the constant heavy 1 (CH1) and constant light (CL) domains of one antibody are exchanged (Antibody A), keeping the variable heavy (VH) and variable light (VL) domains consistent. The exchange of the CH1 and CL domains ensured that the modified antibody (Antibody A) light chain would only efficiently dimerize with the modified antibody (antibody A) heavy chain, while the unmodified antibody (Antibody B) light chain would only efficiently dimerize with the unmodified antibody (Antibody B) heavy chain; and thus only the desired bispecific CrossMab would be efficiently formed (see e.g., Cain. C. SciBX 4(28); doi:10.1038/scibx.2011.783, the contents of which are incorporated by reference herein).

### Common Heavy Chain

An exemplary method of enhancing the formation of a desired bispecific antibody from a mixture of monomers is by providing a common variable heavy chain to interact with each of the heteromeric variable light chain regions of the bispecific antibody. Compositions and methods of producing bispecific antibodies with a common heavy chain are disclosed in, e.g., US20120184716, US20130317200, and US20160264685A1, the contents of each of which is incorporated by reference herein.

### Amino Acid Modifications

Alternative compositions and methods of producing multispecific antibodies with correct light chain pairing include various amino acid modifications. For example. Zymeworks describes heterodimers with one or more amino acid modifications in the CH1 and/or CL domains, one or more amino acid modifications in the VH and/or VL domains, or a combination thereof, which are part of the interface between the light chain and heavy chain and create preferential pairing between each heavy chain and a desired light chain such that when the two heavy chains and two light chains of the heterodimer pair are co-expressed in a cell, the heavy chain of the first heterodimer preferentially pairs with one of the light chains rather than the other (see e.g., WO2015181805). Other exemplary methods are described in WO2016026943 (Argen-X), US20150211001, US20140072581A1, US20160039947A1, and US20150368352.

### Lambda/Kappa Formats

Multispecific molecules (e.g., multispecific antibody molecules) that include the lambda light chain polypeptide and a kappa light chain polypeptides, can be used to allow for heterodimerization. Methods for generating bispecific antibody molecules comprising the lambda light chain polypeptide and a kappa light chain polypeptides are disclosed in PCT/US17/53053 filed on Sep. 22, 2017 and designated publication number WO 2018/057955, incorporated herein by reference in its entirety.

In some embodiments, the multispecific molecule includes a multispecific antibody molecule, e.g., an antibody molecule comprising two binding specificities, e.g., a bispecific antibody molecule. The multispecific antibody molecule includes:
a lambda light chain polypeptide 1 (LLCP1) specific for a first epitope;
a heavy chain polypeptide 1 (HCP1) specific for the first epitope;
a kappa light chain polypeptide 2 (KLCP2) specific for a second epitope; and
a heavy chain poly peptide 2 (HCP2) specific for the second epitope.

“Lambda light chain polypeptide 1 (LLCP1)”, as that term is used herein, refers to a polypeptide comprising sufficient light chain (LC) sequence, such that when combined with a cognate heavy chain variable region, can mediate specific binding to its epitope and complex with an HCP1. In some embodiments, it comprises all or a fragment of a CH1 region. In some embodiments, an LLCP1 comprises LC-CDR1, LC-CDR2, LC-CDR3, FR1, FR2, FR3, FR4, and CH1, or sufficient sequence therefrom to mediate specific binding of its epitope and complex with an HCP1, LLCP1, together with its HCP1, provide specificity for a first epitope (while KLCP2, together with its HCP2, provide specificity for a second epitope). As described elsewhere herein, LLCP1 has a higher affinity for HCP1 than for HCP2.

“Kappa light chain polypeptide 2 (KLCP2)”, as that term is used herein, refers to a polypeptide comprising sufficient light chain (LC) sequence, such that when combined with a cognate heavy chain variable region, can mediate specific binding to its epitope and complex with an HCP2. In some embodiments, it comprises all or a fragment of a CH1 region. In some embodiments, a KLCP2 comprises LC-CDR1, LC-CDR2, LC-CDR3, FR1, FR2, FR3, FR4, and CH1, or sufficient sequence therefrom to mediate specific binding of its epitope and complex with an HCP2, KLCP2, together with its HCP2, provide specificity for a second epitope (while LLCP1, together with its HCP1, provide specificity for a first epitope).

“Heavy chain polypeptide 1 (HCP1)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In some embodiments, it comprises all or a fragment of a CH1 region. In some embodiments, it comprises all or a fragment of a CH2 and/or CH3 region. In some embodiments, an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an LLCP1, (ii) to complex preferentially, as described herein to LLCP1 as opposed to KLCP2; and (iii) to complex preferentially, as described herein, to an HCP2, as opposed to another molecule of HCP1, HCP1, together with its LLCP1, provide specificity for a first epitope (while KLCP2, together with its HCP2, provide specificity for a second epitope).

“Heavy chain polypeptide 2 (HCP2)” as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In some embodiments, it comprises all or a fragment of a CH1 region. In some embodiments, it comprises all or a fragment of a CH2 and/or CH3 region. In some embodiments, an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an KLCP2, (ii) to complex preferentially, as described herein to KLCP2 as opposed to LLCP1; and (iii) to complex preferentially, as described herein, to an HCP1, as opposed to another molecule of HCP2, HCP2, together with its KLCP2, provide specificity for a second epitope (while LLCP1, together with its HCP1, provide specificity for a first epitope).

In some embodiments, in the multifunctional poly peptide molecule as described herein:
LLCP1 has a higher affinity for HCP1 than for HCP2; and/or
KLCP2 has a higher affinity for HCP2 than for HCP1.

in some embodiments, the affinity of LLCP1 for HCP1 is sufficiently greater than its affinity for HCP2, such that under preselected conditions, e.g., in aqueous buffer, e.g., at pH 7, in saline, e.g., at pH 7, or under physiological conditions, at least 75, 80, 90, 95.98, 99, 99.5, or 99.9% of the multispecific antibody molecule molecules have a LLCP1 complexed, or interfaced with, a HCP1.

In some embodiments, in the multifunctional polypeptide molecule as described herein:
the HCP1 has a greater affinity for HCP2, than for a second molecule of HCP1; and/or
the HCP2 has a greater affinity for HCP1, than for a second molecule of HCP2.

In some embodiments, the affinity of HCP1 for HCP2 is sufficiently greater than its affinity for a second molecule of HCP1, such that under preselected conditions, e.g., in aqueous buffer, e.g., at pH 7, in saline, e.g., at pH 7, or under physiological conditions, at least 75%, 80, 90, 95, 98, 99 99.5 or 99.9% of the multispecific antibody molecule molecules have a HCP1 complexed, or interfaced with, a HCP2.

In another aspect, described herein is a method for making, or producing, a multispecific antibody molecule. The method includes:
(i) providing a first heavy chain polypeptide (e.g. a heavy chain polypeptide comprising one, two, three or all of a first heavy chain variable region (first VH), a first CH1, a first heavy chain constant region (e.g., a first CH2, a first CH3, or both));
(ii) providing a second heavy chain polypeptide (e.g., a heavy chain polypeptide comprising one, two, three or all of a second heavy chain variable region (second VH), a second CH1, a second heavy chain constant region (e.g., a second CH2, a second CH3, or both));
(iii) providing a lambda chain polypeptide (e.g., a lambda light variable region (VLλ), a lambda light constant chain (VLλ), or both) that preferentially associates with the first heavy chain polypeptide (e.g., the first VH); and
(iv) providing a kappa chain polypeptide (e.g., a lambda light variable region (VLλ), a lambda light constant chain (VLλ), or both) that preferentially associates with the second heavy chain polypeptide (e.g., the second VH), under conditions where (i)-(iv) associate.

In some embodiments, the first and second heavy chain polypeptides form an Fc interface that enhances heterodimerization.

In some embodiments, (i)-(iv) (e.g., nucleic acid encoding (i)-(iv)) are introduced in a single cell. e.g., a single mammalian cell, e.g., a CHO cell. In some embodiments. (i)-(iv) are expressed in the cell. In some embodiments, (i)-(iv) (e.g., nucleic acid encoding (i)-(iv)) are introduced in different cells, e.g., different mammalian cells, e.g., two or more CHO cell. In some embodiments, (i)-(iv) are expressed in the cells.

In some embodiments, the method further comprises purifying a cell-expressed antibody molecule, e.g., using a lambda- and/or -kappa-specific purification, e.g., affinity chromatography.

In some embodiments, the method further comprises evaluating the cell-expressed multispecific antibody molecule. For example, the purified cell-expressed multispecific antibody molecule can be analyzed by techniques known in the art, include mass spectrometry. In some embodiments, the purified cell-expressed antibody molecule is cleaved, e.g., digested with papain to yield the Fab moieties and evaluated using mass spectrometry.

In some embodiments, the method produces correctly paired kappa/lambda multispecific, e.g., bispecific, antibody molecules in a high yield, e.g., at least 75%, 80, 90, 95, 98, 99, 99.5 or 99.9%.

In other embodiments, the multispecific, e.g., a bispecific, antibody molecule that includes:
(i) a first heavy chain polypeptide (HCP1) (e.g., a heavy chain polypeptide comprising one, two, three or all of a first heavy chain variable region (first VH), a first CH1, a first heavy chain constant region (e.g., a first CH2, a first CH3, or both)), e.g., wherein the HCP1 binds to a first epitope;
(ii) a second heavy chain polypeptide (HCP2) (e.g., a heavy chain polypeptide comprising one, two, three or all of a second heavy chain variable region (second VH), a second CH1, a second heavy chain constant region (e.g., a second CH2, a second CH3, or both)), e.g., wherein the HCP2 binds to a second epitope;
(iii) a lambda light chain polypeptide (LLCP1) (e.g., a lambda light variable region (VLλ), a lambda light constant chain (VLλ), or both) that preferentially associates with the first heavy chain polypeptide (e.g., the first VH), e.g., wherein the LLCP1 binds to a first epitope, and
(iv) a kappa light chain polypeptide (KLCP2) (e.g., a kappa light variable region (VLκ), a kappa light constant chain (VLκ), or both) that preferentially associates with the second heavy chain polypeptide (e.g., the second VH), e.g., wherein the KLCP2 binds to a second epitope.

In some embodiments, the first and second heavy chain polypeptides form an Fc interface that enhances heterodimerization. In some embodiments, the multispecific antibody molecule has a first binding specificity that includes a hybrid VLX-CL, heterodimerized to a first heavy chain variable region connected to the Fc constant, CH2-CH3 domain (having a knob modification) and a second binding specificity that includes a hybrid VLκ-CLκ heterodimerized to a second heavy chain variable region connected to the Fc constant, CH2-CH3 domain (having a hole modification).

### Multispecific or Multifunctional Antibody Molecules

Exemplary structures of multispecific and multifunctional molecules defined herein are described throughout. Exemplary structures are further described in: Weidle U et al. (2013) The Intriguing Options of Multispecific Antibody Formats for Treatment of Cancer. Cancer Genomics & Proteomics 10: 1-18 (2013); and Spiess C et al. (2015) Alternative molecular formats and therapeutic applications for bispecific antibodies. Molecular Immunology 67: 95-106; the full contents of each of which is incorporated by reference herein).

In some embodiments, multispecific antibody molecules can comprise more than one antigen-binding site, where different sites are specific for different antigens. In some embodiments, multispecific antibody molecules can bind more than one (e.g., two or more) epitopes on the same antigen. In some embodiments, multispecific antibody molecules comprise an antigen-binding site specific for a target cell (e.g., cancer cell) and a different antigen-binding site specific for an immune effector cell. In some embodiments, the multispecific antibody molecule is a bispecific antibody molecule. Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange. SEEDbody, triomab, LUZ-Y. Fcab, κλ-body, orthogonal Fab. See Spiess et al. Mol. Immunol. 67(2015):95-106. Exemplary BsIgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2. In some embodiments. BsIgG comprises heavy chains that are engineered for heterodimerization. For example, heavy chains can be engineered for heterodimerization using a “knobs-into-holes” strategy, a SEED platform, a common heavy chain (e.g., in κλ-bodies), and use of heterodimeric Fc regions. See Spiess et al. Mol. Immunol. 67(2015):95-106. Strategies that have been used to avoid heavy chain pairing of homodimers in BsIgG include knobs-in-holes, duobody, azymetric, charge pair, HA-TF, SEEDbody. and differential protein A affinity. See Id BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution.

IgG appended with an additional antigen-binding moiety is another format of bispecific antibody molecules. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). See Id. Examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. An example of an IgG-scFv is MM-141 (Merrimack Pharmaceuticals), which binds IGF-1R and HER3. Examples of DVD-Ig include ABT-981 (AbbVie), which binds IL-1α and IL-1β; and ABT-122 (AbbVie), which binds TNF and IL-17A.

Bispecific antibody fragments (BsAb) are a format of bispecific antibody molecules that lack some or all of the antibody constant domains. For example, some BsAb lack an Fc region. In some embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody. See Id. For example, the BiTE format comprises tandem scFvs, where the component scFys bind to CD3 on T cells and a surface antigen on cancer cells.

Bispecific fusion proteins include antibody fragments linked to other proteins, e.g., to add additional specificity and/or functionality. An example of a bispecific fusion protein is an immTAC, which comprises an anti-CD3 scFv linked to an affinity-matured T-cell receptor that recognizes HLA-presented peptides. In some embodiments, the dock-and-lock (DNL) method can be used to generate bispecific antibody molecules with higher valency. Also, fusions to albumin binding proteins or human serum albumin can be extend the serum half-life of antibody fragments. See Id.

In some embodiments, chemical conjugation, e.g., chemical conjugation of antibodies and/or antibody fragments, can be used to create BsAb molecules. See Id. An exemplary bispecific antibody conjugate includes the CovX-body format, in which a low molecular weight drug is conjugated site-specifically to a single reactive lysine in each Fab arm or an antibody or fragment thereof. In some embodiments, the conjugation improves the serum half-life of the low molecular weight drug. An exemplary CovX-body is CVX-241 (NCT01004822), which comprises an antibody conjugated to two short peptides inhibiting either VEGF or Ang2. See Id.

The antibody molecules can be produced by recombinant expression, e.g., of at least one or more component, in a host system. Exemplary host systems include eukaryotic cells (e.g., mammalian cells, e.g., CHO cells, or insect cells, e.g., SF9 or S2 cells) and prokaryotic cells (e.g., E. coli). Bispecific antibody molecules can be produced by separate expression of the components in different host cells and subsequent purification/assembly. Alternatively, the antibody molecules can be produced by expression of the components in a single host cell. Purification of bispecific antibody molecules can be performed by various methods such as affinity chromatography, e.g., using protein A and sequential pH elution. In other embodiments, affinity tags can be used for purification, e.g., histidine-containing tag, myc tag, or streptavidin tag.

### Exemplary Bispecific Molecules

In an aspect, a multispecific molecule as described herein comprises a sequence as described herein, e.g., a sequence chosen from SEQ ID NOs: 1004-1007, 3275-3277, 3286, or 3287, or a sequence with at least 85%, 90%, 955, 96%, 97%, 98%, 99%/0 or more identity thereto. In some embodiments, a multispecific molecule as described herein comprises a leader sequence comprising the amino acid sequence of SEQ ID NO: 3288. In some embodiments, a multispecific molecule as described herein does not comprise a leader sequence comprising the amino acid sequence of SEQ ID NO: 3288.

Molecule F: aCD19×aVb6.5: Molecule F comprises a heavy chain comprising the amino acid sequence of SEQ ID NO: 1004 and a light chain comprising the amino acid sequence of SEQ ID NO: 1005.

Molecule F.1 (heavy chain) (TCRVbeta6_5 scFv/anti-CD19 heavy chain)
SEQ ID NO: 1004
METDTLLLWVLLLWVPGSTGQVQLVQSGAEVKKPGSSVKVSCKASGYSFTTYYIHWVRQAPGQ
GLEWMGWFFPGSGNIKYNEKFKGRVTITADTSTSTAYMELSSLRSEDTAVYYCAGSYYSYDVLD
YWGQGTTVTVSSGGGGSGGGGSGGGGSGGGGSDIQMTQSPSFLSASVGDRVTITCKASQNVGIN
VVWHQQKPGKAPKALIYSSSHRYSGVPSRFSGSGSGTEFTLTISSLQPEDFATYFCQQFKSYPLTF
GQGTKLEIKGGGGSQVTLRESGPALVKPTQTLTLTCTFSGFSLSTSGMGVGWIRQPPGKALEWLA
HIWWDDDKRYNPALKSRLTISKDTSKNQVFLTMTNMDPVDTATYYCARMELWSYYFDYWGQG
TTVTVSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTKPREEQYASTYRVVSV
LTVLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEALH
NHYTQKSLSLSPGK
Molecule F.2 (light chain) (anti-CD19 light chain)
SEQ ID NO: 1005
METPAQLLFLLLLWLPDTTGENVLTQSPATLSLSPGERATLSCSASSSVSYMHWYQQKPGQAPRL
LIYDTSKLASGIPARFSGSGSGTDHTLTISSLEPEDFAVYYCFQGSVYPFTFGQGTKLEIKRTVAAP
SVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC

In an aspect, a multispecific molecule as described herein comprises SEQ ID NO: 1004 and/or SEQ ID NO: 1005 or a sequence with at least 85%, 90%, 955, 96%, 97%, 98%, 99% or more identity thereto.

Molecule G: aBCMA×aVb6.5: Molecule G comprises a heavy chain comprising the amino acid sequence of SEQ ID NO: 1006 and a light chain comprising the amino acid sequence of SEQ ID NO: 1007.

Molecule G.1 (heavy chain)
SEQ ID NO: 1006
METDTLLLWVLLLWVPGSTGQVQLVQSGAEVKKPGSSVKVSCKASGYSFTTYYIHWVRQAPGQ
GLEWMGWFFPGSGNIKYNEKFKGRVTITADTSTSTAYMELSSLRSEDTAVYYCAGSYYSYDVLD
YWGQGTTVTVSSGGGGSGGGGSGGGGSGGGGSDIQMTQSPSFLSASVGDRVTITCKASQNVGIN
VVWHQQKPGKAPKALIYSSSHRYSGVPSRFSGSGSGTEFTLTISSLQPEDFATYFCQQFKSYPLTF
GQGTKLEIKGGGGSQVQLVESGGGVVQPGRSLRLSCAASGIDFSRYWMSWVRQAPGKGLEWVG
EINPDSSTINYAPSLKDRFTISRDNSKNTLYLQMSSLRAEDTAVYYCASLYYDYGDAMDYWGQG
TTVTVSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTKPREEQYASTYRVVSV
LTVLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEALH
NRFTQKSLSLSPGK
Molecule G.2 (light chain)
SEQ ID NO: 1007
METDTLLLWVLLLWVPGSTGDIQMTQSPSSLSASVGDRVTITCKASQSVDSNVAWYQQKPEKAP
KALIFSASLRFSGVPSRFSGSGSGTDFTLTISSLQPEDFATYFCQQYNNYPLTFGQGTKLEIKRTVA
APSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC

In an aspect, a multispecific molecule as described herein comprises SEQ ID NO: 1006 and/or SEQ ID NO: 1007 or a sequence with at least 85%, 90%, 955, 96%, 97%, 98%, 99% or more identity thereto.

Molecule H: aBCMA×aTCRvbeta6_5: Molecule H comprises a first heavy chain comprising the amino acid sequence of SEQ ID NO: 3275, a light chain comprising the amino acid sequence of SEQ ID NO: 3277, and a second heavy chain comprising the amino acid sequence of SEQ ID NO: 3276.

Molecule H.1 (anti-BCMA heavy chain)
SEQ ID NO: 3275
METDTLLLWVLLLWVPGSTGQVQLVESGGGVVQPGRSLRLSCAASGIDFSRYWMSWVRQAPG
KGLEWVGEINPDSSTINYAPSLKDRFTISRDNSKNTLYLQMSSLRAEDTAVYYCASLYYDYGDA
MDYWGQGTTVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHTCPPCPAPE
LLGGPSVFLFPPKPKDTLMISRTPEVTCVVVDVSHEDPEVKFNWYVDGVEVHNAKTKPREEQYN
ATYRVVSVLTVLHQDWLNGKEYKCKVSNKALPAPIEKTISKAKGQPREPQVYTLPPCREEMTKN
QVSLWCLVKGFYPSDIAVEWESNGQPENNYKTTPPVLDSDGSFFLYSKLTVDKSRWQQGNVFSC
SVMHEALHNHYTQKSLSLSPGK
Molecule H.2 (TCRvbeta_6_5 scFv humanized)
SEQ ID NO: 3276
METDTLLLWVLLLWVPGSTGQVQLVQSGAEVKKPGSSVKVSCKASGYSFTTYYIHWVRQAPGQ
GLEWMGWFFPGSGNIKYNEKFKGRVTITADTSTSTAYMELSSLRSEDTAVYYCAGSYYSYDVLD
YWGQGTTVTVSSGGGGSGGGGSGGGGSGGGGSDIQMTQSPSFLSASVGDRVTITCKASQNVGIN
VVWHQQKPGKAPKALIYSSSHRYSGVPSRFSGSGSGTEFTLTISSLQPEDFATYFCQQFKSYPLTF
GQGTKLEIKGGGGSGGGGSDKTHTCPPCPAPELLGGPSVFLFPPKPKDTLMISRTPEVTCVVVDVS
HEDPEVKFNWYVDGVEVHNAKTKPREEQYASTYRVVSVLTVLHQDWLNGKEYKCKVSNKALP
APIEKTISKAKGQPREPQVCTLPPSREEMTKNQVSLSCAVKGFYPSDIAVEWESNGQPENNYKTTP
PVLDSDGSFFLVSKLTVDKSRWQQGNVFSCSVMHEALHNHYTQKSLSLSPGK
Molecule H.3 (anti-BCMA light chain)
SEQ ID NO: 3277
METDTLLLWVLLLWVPGSTGDIQMTQSPSSLSASVGDRVTITCKASQSVDSNVAWYQQKPEKAP
KALIFSASLRFSGVPSRFSGSGSGTDFTLTISSLQPEDFATYFCQQYNNYPLTFGQGTKLEIKRTVA
APSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC

In an aspect, a multispecific molecule as described herein comprises SEQ ID NO: 3275. SEQ ID NO: 3276, and/or SEQ ID NO: 3277 or a sequence with at least 85%, 90%, 955, 96%, 97%, 98%, 99% or more identity thereto.

Molecule I: half arm BCMA Fab with c-terminal scFv TCRvbeta: Molecule I comprises a first heavy chain comprising the amino acid sequence of SEQ ID NO: 3286, a light chain comprising the amino acid sequence of SEQ ID NO: 3277, and a second heavy chain comprising the amino acid sequence of SEQ ID NO: 3287.

Molecule I.1 (heavy chain 1)
SEQ ID NO: 3286
METDTLLLWVLLLWVPGSTGQVQLVESGGGVVQPGRSLRLSCAASGIDFSRYWMSWVRQAPG
KGLEWVGEINPDSSTINYAPSLKDRFTISRDNSKNTLYLQMSSLRAEDTAVYYCASLYYDYGDA
MDYWGQGTTVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHTCPPCPAPE
LLGGPSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPCREEMTKN
QVSLWCLVKGFYPSDIAVEWESNGQPENNYKTTPPVLDSDGSFFLYSKLTVDKSRWQQGNVFSC
SVMHEALHNHYTQKSLSLSPGKGGGGSGGGGSGGGGSEVQLVESGGGLVQPGGSLRLSCAASG
FTFSTYAMNWVRQAPGKGLEWVSRIRSKYNNYATYYADSVKDRFTISRDDSKNTLYLQMNSLK
TEDTAVYYCVRHGNFGNSYVSWFAYWGQGTLVTVSSGGGGSGGGGSGGGGGGGGSQAVVT
QEPSLTVSPGGTVTLTCRSSTGAVTTSNYANWVQQKPGQAPRGLIGGTNKRAPWTPARFSGSLL
GGKAALTLSGAQPEDEAEYYCALWYSNLWVFGGGTKLTVL
Molecule I.2 (light chain)
SEQ ID NO: 3277
METDTLLLWVLLLWVPGSTGDIQMTQSPSSLSASVGDRVTITCKASQSVDSNVAWYQQKPEKAP
KALIFSASLRFSGVPSRFSGSGSGTDFTLTISSLQPEDFATYFCQQYNNYPLTFGQGTKLEIKRTVA
APSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Molecule I.3 (heavy chain 2)
SEQ ID NO: 3287
METDTLLLWVLLLWVPGSTGDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKA
LPAPIEKTISKAKGQPREPQVCTLPPSREEMTKNQVSLSCAVKGFYPSDIAVEWESNGQPENNYKT
TPPVLDSDGSFFLVSKLTVDKSRWQQGNVFSCSVMHEALHNHYTQKSLSLSPGK

In an aspect, a multispecific molecule as described herein comprises SEQ ID NO: 3286, SEQ ID NO: 3277, and/or SEQ ID NO: 3287 or a sequence with at least 85%, 90%, 955, 9%, 97%, 98%, 99% or more identity thereto.

In some embodiments, the multispecific or multifunctional molecule as described herein binds to immune cells. In some embodiments, the multispecific or multifunctional molecule as described herein binds to subsets of immune cells. In some embodiments, the multispecific or multifunctional molecule as described herein binds to T cells. In some embodiments, the multispecific or multifunctional molecule as described herein binds to gamma/delta T cells. In some embodiments, the multispecific or multifunctional molecule as described herein binds to NKT cells. In some embodiments, the multispecific or multifunctional molecule as described herein binds to T cells via a binding moiety. Exemplary binding moieties include, but are not limited to, the binding moieties that bind to TRBC1, TRBC2, CD3, TRAC, TRAV subtypes, CD4, CD8, CD2, CD28, 41BB, PD1, CTLA4, OX40, TIM3, or LAG3. In some embodiments, the multispecific or multifunctional molecule as described herein binds to T cells via a binding moiety that binds to TRBC1. In some embodiments, the multispecific or multifunctional molecule as described herein binds to T cells via a binding moiety that binds to TRBC2. In some embodiments, the multispecific or multifunctional molecule as described herein comprises a binding moiety that binds to TRBC1. In some embodiments, the multispecific or multifunctional molecule as described herein comprises a binding moiety that binds to TRBC2.

### Linkers

The multispecific or multifunctional molecule as described herein can further include a linker, e.g., a linker between one or more of: the antigen binding domain and the cytokine molecule, the antigen binding domain and the immune cell engager, the antigen binding domain and the stromal modifying moiety, the cytokine molecule and the immune cell engager, the cytokine molecule and the stromal modifying moiety, the immune cell engager and the stromal modifying moiety, the antigen binding domain and the immunoglobulin chain constant region, the cytokine molecule and the immunoglobulin chain constant region, the immune cell engager and the immunoglobulin chain constant region, or the stromal modifying moiety and the immunoglobulin chain constant region. In some embodiments, the linker is chosen from: a cleavable linker, a non-cleavable linker, a peptide linker, a flexible linker, a rigid linker, a helical linker, or a non-helical linker, or a combination thereof.

In some embodiments, the multispecific molecule can include one, two, three or four linkers, e.g., a peptide linker. In some embodiments, the peptide linker includes Gly and Scr. In some embodiments, the peptide linker is selected from GGGGS (SEQ ID NO: 3307); GGGGSGGGGS (SEQ ID NO: 3308); GGGGSGGGGSGGGGS (SEQ ID NO: 3309); DVPSGPGGGGGSGGGGS (SEQ ID NO: 3310); and GGGGSGGGGSGGGGGS (SEQ ID NO: 3643). In some embodiments, the peptide linker is a A(EAAAK)nA (SEQ ID NO: 3437) family of linkers (e.g., as described in Protein Eng. (2001) 14 (8): 529-532). These are stiff helical linkers with n ranging from 2-5. In some embodiments, the peptide linker is selected from AEAAAKEAAAKAAA (SEQ ID NO: 3314); AEAAAKEAAAKEAAAKAAA (SEQ ID NO: 3315): AEAAAKEAAAKEAAAKEAAAKAAA (SEQ ID NO: 3316); and AEAAAKEAAAKEAAAKEAAAKEAAAKAAA (SEQ ID NO: 3317).

### Nucleic Acids

Described herein, in certain embodiments, is an isolated nucleic acid molecule comprising a nucleotide sequence having at least 75%, 80%, 85%, 90%, 95%, 99%, 99.5%, 99.9%, or 100% sequence identity to the nucleotide sequence encoding the multifunctional polypeptide molecule as described herein.

Nucleic acids encoding the aforementioned antibody molecules, e.g., anti-TCRβV antibody molecules, multispecific or multifunctional molecules are also disclosed.

In certain embodiments, the invention features nucleic acids comprising nucleotide sequences that encode heavy and light chain variable regions and CDRs or hypervariable loops of the antibody molecules, as described herein. For example, the invention features a first and second nucleic acid encoding heavy and light chain variable regions, respectively, of an antibody molecule chosen from one or more of the antibody molecules as described herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding at least one, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein).

In certain embodiments, the nucleic acid can comprise a nucleotide sequence encoding a cytokine molecule, an immune cell engager, or a stromal modifying moiety as described herein.

In another aspect, the application features host cells and vectors containing the nucleic acids described herein. The nucleic acids may be present in a single vector or separate vectors present in the same host cell or separate host cell, as described in more detail hereinbelow.

### Vectors

Described herein, in certain embodiments, is a vector comprising one or more of the nucleic acid molecules as described herein.

Further provided herein are vectors comprising the nucleotide sequences encoding antibody molecules, e.g., anti-TCRβV antibody molecules, or a multispecific or multifunctional molecule described herein. In some embodiments, the vectors comprise nucleic acid sequences encoding antibody molecules. e.g., anti-TCRβV antibody molecules, or multispecific or multifunctional molecule described herein. In some embodiments, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus. Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Host Cells

Described herein, in certain embodiments, is a cell comprising the nucleic acid as described herein or the vector as described herein.

In another aspect, described herein are host cells and vectors containing the nucleic acids. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

In some embodiments, described herein are host cells comprising a nucleic acid encoding an antibody molecule as described herein.

In some embodiments, described herein are the host cells genetically engineered to comprise nucleic acids encoding the antibody molecule.

In some embodiments, the host cells are genetically engineered by using an expression cassette. The phrase “expression cassette.” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

In some embodiments, described herein are host cells comprising the vectors described herein. The cell can be, but is not limited to, a eukaryotic cell, a bacterial cell, an insect cell, or a human cell. Suitable eukaryotic cells include, but are not limited to, Vero cells. HeLa cells. COS cells, CHO cells, HEK293 cells. BHK cells and MDCKII cells. Suitable insect cells include, but are not limited to, Sf9 cells.

Method of Expanding Cells with Anti-TCRVB Antibodies

Any of the compositions and methods described herein can be used to expand an immune cell population. An immune cell provided herein includes an immune cell derived from a hematopoietic stem cell or an immune cell derived from a non-hematopoietic stem cell, e.g., by differentiation or de-differentiation.

An immune cell includes a hematopoietic stem cell, progeny thereof and/or cells that have differentiated from said HSC, e.g., lymphoid cells or myeloid cells. An immune cell can be an adaptive immune cell or an innate immune cell. Examples of immune cells include T cells, B cells, Natural Killer cells, Natural Killer T cells, neutrophils, dendritic cells, monocytes, macrophages, and granulocytes.

In some embodiments, an immune cell is a T cell. In some embodiments, a T cell includes a CD4+ T cell, a CD8+ T cell, a TCR alpha-beta T cell, a TCR gamma-delta T cell. In some embodiments, a T cell comprises a memory T cell (e.g., a central memory T cell, or an effector memory T cell (e.g., a TEMRA) or an effector T cell. In some embodiments, a T cell comprises a tumor infiltrating lymphocyte (TIL).

In some embodiments, an immune cell is an NK cell.

In some embodiments, an immune cell is a TIL. TILs are immune cells (e.g., T cells, B cells or NK cells) that can be found in a tumor or around a tumor (e.g., in the stroma or tumor microenvironment of a tumor), e.g., a solid tumor, e.g., as described herein. TILs can be obtained from a sample from a subject having cancer, e.g., a biopsy or a surgical sample. In some embodiments, TILs can be expanded using a method as described herein. In some embodiments, a population of expanded TILs, e.g., expanded using a method as described herein, can be administered to a subject to treat a disease, e.g., a cancer.

In some embodiments, immune cells, e.g., T cells (e.g., TILs), can be obtained from a unit of blood collected from a subject using any number of techniques known to the skilled artisan, such as Ficoll™ separation. In one aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In some embodiments, the cells are washed with phosphate buffered saline (PBS). In an alternative embodiment, the wash solution lacks calcium and may lack magnesium or may lack many if not all divalent cations. The methods described herein can include more than one selection step, e.g., more than one depletion step.

In some embodiments, the methods of the application can utilize culture media conditions comprising DMEM, DMEM F12, RPMI 1640, and/or AIM V media. The media can be supplemented with glutamine, HEPES buffer (e.g., 10 mM), serum (e.g., heat-inactivated serum, e.g., 10%), and/or beta mercaptoethanol (e.g., 55 uM). IN some embodiments, the culture conditions as described herein comprise one or more supplements, cytokines, growth factors, or hormones. In some embodiments, the culture condition comprises one or more of IL-2, IL-15, or IL-7, or a combination thereof.

Immune effector cells such as T cells may be activated and expanded generally using methods as described, for example, in U.S. Pat. No. 6,352,694; 6,534,055; or 6,905,680. Generally, a population of immune cells, may be expanded by contact with an agent that stimulates a CD3/TCR complex associated signal and a ligand that stimulates a costimulatory molecule on the surface of the T cells; and/or by contact with a cytokine, e.g., IL-2, IL-15 or IL-7. T cell expansion protocols can also include stimulation, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besançon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 199)).

A TIL population can also be expanded by methods known in the art. For example, a population of TILs can be expanded as described in Hall et al., Journal for ImmunoTherapy of Cancer (2016) 4:61, the entire contents of which are hereby incorporated by reference. Briefly, TILs can be isolated from a sample by mechanical and/or physical digestion. The resultant TIL population can be stimulated with an anti-CD3 antibody in the presence of non-dividing feeder cells. In some embodiments, the TIL population can be cultured, e.g., expanded, in the presence of IL-2, e.g., human IL-2. In some embodiments, the TIL cells can be cultured, e.g., expanded for a period of at least 1-21 days, e.g., at least 1, 2, 3, 4, 5, 6, 7, 8, 9, 10, 11, 12, 13, 14, 15, 16, 17, 18, 19, 20 or 21 days.

As described herein, in some embodiments, an immune cell population (e.g., a T cell (e.g., a T_{EMRA} cell or a TIL population) can be expanded by contacting the immune cell population with an anti-TCRVB antibody, e.g., as described herein.

In some embodiments, the expansion occurs in vivo, e.g., in a subject. In some embodiments, a subject is administered the multispecific or multifunctional molecules comprising TCRβV-binding moieties as described herein resulting in expansion of immune cells in vivo.

In some embodiments, the expansion occurs ex vivo, e.g., in vitro. In some embodiments, cells from a subject, e.g., T cells, e.g., TIL cells, are expanded in vitro with the multispecific or multifunctional molecules as described herein. In some embodiments, the expanded TILs are administered to the subject to treat a disease or a symptom of a disease.

In some embodiments, a method of expansion as described herein results in an expansion of at least 1.1-10 fold, 10-20 fold, or 20-50 fold expansion. In some embodiments, the expansion is at least 1.1, 1.2, 1.3, 1.4, 1.5, 2, 3, 4, 5, 6, 7, 8, 9, 10, 15, 20, 25, 30, 35, 40, 45 or 50 fold expansion.

In some embodiments, a method of expansion as described herein comprises culturing, e.g., expanding, the cells for at least about 4 hours, 6 hours, 10 hours, 12 hours, 15 hours, 18 hours, 20 hours, or 22 hours. In some embodiments, a method of expansion as described herein comprises culturing, e.g., expanding, the cells for at least 1, 2, 3, 4, 5, 6, 7, 8, 9, 10, 11, 12, 13, 14, 15, 16, 17, 18, 19, 20 or 21 days. In some embodiments, a method of expansion as described herein comprises culturing, e.g., expanding, the cells for at least about 1 week, 2 weeks, 3 weeks, 4 weeks, 5 weeks, 6 weeks, 7 weeks or 8 weeks.

In some embodiments, a method of expansion as described herein is performed on immune cells obtained from a healthy subject.

In some embodiments, a method of expansion as described herein is performed on immune cells (e.g., TILs) obtained from a subject having a disease, e.g., a cancer, e.g., a solid tumor as described herein.

In some embodiments, a method of expansion as described herein further comprises contacting the population of cells with an agent, that promotes, e.g., increases, immune cell expansion. In some embodiments, the agent comprises an immune checkpoint inhibitor, e.g., a PD-1 inhibitor, a LAG-3 inhibitor, a CTLA4 inhibitor, or a TIM-3 inhibitor. In some embodiments, the agent comprises a 4-1BB agonist, e.g., an anti-4-1BB antibody.

Without wishing to be bound by theory, in some embodiments, the multispecific or multifunctional molecules as described herein can expand, e.g., selectively or preferentially expand. T cells expressing a T cell receptor (TCR) comprising a TCR alpha and/or TCR beta molecule, e.g., TCR alpha-beta T cells (αβ cells). In some embodiments, the multispecific or multifunctional molecules as described herein do not expand, or induce proliferation of T cells expressing a TCR comprising a TCR gamma and/or TCR delta molecule, e.g., TCR gamma-delta T cells (γδ T cells). In some embodiments, the multispecific or multifunctional molecules as described herein selectively or preferentially expand αβ T cells over γδ T cells.

Without wishing to be bound by theory, it is believed that, in some embodiments. γδ T cells are associated with cytokine release syndrome (CRS) and/or neurotoxicity (NT). In some embodiments, the multispecific or multifunctional molecules as described herein result in selective expansion of non-γδ T cells, e.g., expansion of αβ T cells, thus reducing CRS and/or NT.

In some embodiments, any of the compositions or methods as described herein result in an immune cell population having a reduction of, e.g., depletion of, γδ T cells. In some embodiments, the immune cell population is contacted with an agent that reduces, e.g., inhibits or depletes, γδ T cells, e.g., an anti-IL-17 antibody or an agent that binds to a TCR gamma and/or TCR delta molecule.

### CRS Grading

In some embodiments, CRS (Cytokine Release Syndrome) can be graded in severity from 1-5 as follows. Grades 1-3 are less than severe CRS. Grades 4-5 are severe CRS. For Grade 1 CRS, only symptomatic treatment is needed (e.g., nausea, fever, fatigue, myalgias, malaise, headache) and symptoms are not life threatening. For Grade 2 CRS, the symptoms require moderate intervention and generally respond to moderate intervention. Subjects having Grade 2 CRS develop hypotension that is responsive to either fluids or one low-dose vasopressor; or they develop grade 2 organ toxicity or mild respiratory symptoms that are responsive to low flow oxygen (<40% oxygen). In Grade 3 CRS subjects, hypotension generally cannot be reversed by fluid therapy or one low-dose vasopressor. These subjects generally require more than low flow oxygen and have grade 3 organ toxicity (e.g., renal or cardiac dysfunction or coagulopathy) and/or grade 4 transaminitis. Grade 3 CRS subjects require more aggressive intervention, e.g., oxygen of 40% or higher, high dose vasopressor(s), and/or multiple vasopressors. Grade 4 CRS subjects suffer from immediately life-threatening symptoms, including grade 4 organ toxicity or a need for mechanical ventilation. Grade 4 CRS subjects generally do not have transaminitis. In Grade 5 CRS subjects, the toxicity causes death. Sets of criteria for grading CRS are provided herein as Table 5. Table 6, and Table 7. Unless otherwise specified. CRS as used herein refers to CRS according to the criteria of Table 6.

In some embodiments, CRS is graded according to Table 5.

The term “cytokine profile” as used herein, refers to the level and/or activity of on one or more cytokines or chemokines, e.g., as described herein. In some embodiments, a cytokine profile comprises the level and/or activity of a naturally occurring cytokine, a fragment or a variant thereof. In some embodiments, a cytokine profile comprises the level and/or activity of one or more cytokines and/or one or more chemokines (e.g., as described herein). In some embodiments, a cytokine profile comprises the level and/or activity of a naturally occurring cytokine, a fragment or a variant thereof. In some embodiments, a cytokine profile comprises the level and/or activity of a naturally occurring chemokine, a fragment or a variant thereof. In some embodiments, a cytokine profile comprises the level and/or activity of one or more of: IL-2 (e.g., full length, a variant, or a fragment thereof); IL-Ibeta (e.g., full length, a variant, or a fragment thereof); IL-6 (e.g., full length, a variant, or a fragment thereof); TNFα (e.g., full length, a variant, or a fragment thereof); IFNgamma (e.g., full length, a variant, or a fragment thereof) IL-10 (e.g., full length, a variant, or a fragment thereof); IL-4 (e.g., full length, a variant, or a fragment thereof); TNF alpha (e.g., full length, a variant, or a fragment thereof); IL-12p70 (e.g., full length, a variant, or a fragment thereof); IL-13 (e.g., full length, a variant, or a fragment thereof): IL-8 (e.g., full length, a variant, or a fragment thereof); Eotaxin (e.g., full length, a variant, or a fragment thereof); Eotaxin-3 (e.g., full length, a variant, or a fragment thereof); IL-8 (HA) (e.g., full length, a variant, or a fragment thereof); IP-10 (e.g., full length, a variant, or a fragment thereof); MCP-1 (e.g., full length, a variant, or a fragment thereof); MCP-4 (e.g., full length, a variant, or a fragment thereof); MDC (e.g., full length, a variant, or a fragment thereof); MIP-1a (e.g., full length, a variant, or a fragment thereof); MIP-1b (e.g., full length, a variant, or a fragment thereof): TARC (e.g., full length, a variant, or a fragment thereof); GM-CSF (e.g., full length, a variant, or a fragment thereof); IL-12 23p40 (e.g., full length, a variant, or a fragment thereof); IL-15 (e.g., full length, a variant, or a fragment thereof); IL-16 (e.g., full length, a variant, or a fragment thereof): IL-17a (e.g., full length, a variant, or a fragment thereof): IL-1a (e.g., full length, a variant, or a fragment thereof); IL-5 (e.g., full length, a variant, or a fragment thereof); IL-7 (e.g., full length, a variant, or a fragment thereof); TNF-beta (e.g., full length, a variant, or a fragment thereof); or VEGF (e.g., full length, a variant, or a fragment thereof). In some embodiments, a cytokine profile includes secretion of one or more cytokines or chemokines. In some embodiments, a cytokine in a cytokine profile can be modulated, e.g., increased or decreased, by an anti-TCRBV antibody molecule described herein. In some embodiments, the cytokine profile includes cytokines associated with a cytokine storm or cytokine release syndrome (CRS), e.g., IL-6, IL-1beta, TNFalpha and IL-10.

### Pharmaceutical Compositions

Described herein, in certain embodiments, is a pharmaceutical composition comprising the multifunctional polypeptide molecule as described herein, the nucleic acid molecules as described herein, the vector as described herein, or the cell as described herein, and a pharmaceutically acceptable carrier, excipient, or diluent.

Pharmaceutical compositions or formulations comprising the agent, e.g., the multifunctional or multispecific molecules, of the described compositions and for use in any of the described methods can be prepared according to conventional techniques well known in the pharmaceutical industry and described in the published literature. In some embodiments, a pharmaceutical composition or formulation for treating a subject comprises an effective amount of any the multifunctional or multispecific molecules or the compositions as described herein, or a pharmaceutically acceptable salt, solvate, hydrate or ester thereof. The pharmaceutical formulation comprising the multifunctional or multispecific molecules as described herein may further comprise a pharmaceutically acceptable excipient, diluent or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose. The salts can be prepared in situ during the final isolation and purification of the compounds, or separately by reacting the free base form with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In some embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In some embodiments, a pharmaceutical formulation or composition as described herein includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In some embodiments, liposomes also include sterically stabilized liposomes, e.g., liposomes comprising one or more specialized lipids. These specialized lipids result in liposomes with enhanced circulation lifetimes. In some embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In some embodiments, the present disclosure employs a penetration enhancer to effect the efficient delivery of the multifunctional or multispecific molecules or the compositions as described herein, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple multifunctional or multispecific molecules as described herein. In some embodiments, the multifunctional or multispecific molecules or the compositions as described herein is administered in combination with another drug or therapeutic agent.

Described herein is a pharmaceutical composition comprising a population of immune cells, wherein at least 50% of the population of immune cells are central memory (CM) T cells, wherein the CM T cells are induced by binding to a molecule that binds to a T cell receptor beta variable region (TCRβV). In some embodiments, at least 50%, 55%, 60%, 65%, 70%, 75%, 80%, 85%, 90%, 95% or 99% of the population of immune cells are CM T cells. In some embodiments, the population of cells are proliferative or hyper proliferative. In some embodiments, the molecule further comprises a cytokine. In some embodiments, the cytokine is selected from the group consisting of interleukin-2 (IL-2), interleukin-7 (IL-7), interleukin-12 (IL-12), interleukin-15 (IL-15), interleukin-18 (IL-18), interleukin-21 (IL-21), interferon gamma and functional fragments or variants thereof.

In some embodiments, the population of immune cells are cultured in a growth medium ex vivo. In some embodiments, the growth medium may be X-VIVO culture media. In some embodiments, the growth medium comprises a cytokine. In some embodiments, the cytokine is interleukin-2 (IL-2), interleukin-7 (IL-7), interleukin-12 (IL-12), interleukin-15 (IL-15), interleukin-18 (IL-18), interleukin-21 (IL-21), interferon gamma and functional fragments or variants thereof.

In some embodiments, the molecule binds to the TCRβV. In some embodiments, the molecule binds to a germline encoded region of the TCRβV. In some embodiments, the molecule binds to a hypervariable region 4 (HV4) of the TCRβV. In some embodiments, the molecule binds to a complementarity-determining region 2 (CDR2) of the TCRβV.

In some embodiments, the CM T cells are TCRβV+. In some embodiments, at least 20% of the population of immune cells are TCRPV+. In some embodiments, the TCRβV is TCRβV1, TCRβV2, TCRβV3, TCRβV4, TCRβV5, TCRβV6, TCRβV7, TCRβV8, TCRβV9, TCRβV10, TCRβV11, TCRβV12, TCRβV13, TCRβV14, TCRβV15, TCRβV16, TCRβV17, TCRβV18. TCRβV19, TCRβV20, TCRβV21, TCRβV22, TCRβV23, TCRβV24, TCRβV25, TCRDV26, TCRQV27, TCRβV28, TCRβV29 or TCRβV30. In some embodiments, the TCRβV is TCRβV2, TCRβV3-1, TCRβV4-1, TCRβV4-2, TCRβV5-1, TCRβV5-4, TCRβV5-5, TCRβV5-6, TCRβV6-1, TCRβ6-5, TCRβV6-6, TCRβV7-3, TCRβV7-6, TCRβV7-8, TCRβV9, TCRβV11-2, TCRβV19, TCRβV20-1, TCRβV24-1, TCRβV27, TCRβV28, TCRβV29-1 or TCRβV30.

In some embodiments, the CM T cells are CCR7+. In some embodiments, at least 65% of the population of immune cells are CCR7+. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CCR7+. In some embodiments, at most 35% of the population of immune cells are CCR7−. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CCR7−.

In some embodiments, the CM T cells are CD45RA−. In some embodiments, at least 65% of the population of immune cells are CD45RA−. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD45RA−. In some embodiments, at most 35% of the population of immune cells are CD45RA+. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD45RA+.

In some embodiments, the CM T cells are CD95+. In some embodiments, at least 50% of the population of immune cells are CD95+. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD95+. In some embodiments, at most 50% of the population of immune cells are CD95−. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD95−.

In some embodiments, the CM T cells are CCR7+ and CD45RA−. In some embodiments, at least 50% of the population of immune cells are CCR7+ and CD45RA−. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CCR7+ and CD45RA−. In some embodiments, at most 7.7% of the population of immune cells are CCR7− and CD45RA+. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CCR7− and CD45RA+.

In some embodiments, wherein the CM T cells are CD95+ and CCR7+. In some embodiments, wherein at least 50% of the population of immune cells are CD95+ and CCR7+. In some embodiments, wherein at least 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD95+ and CCR7+. In some embodiments, at most 50% of the population of immune cells are CD95− and CCR7−. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD95− and CCR7−.

In some embodiments, the CM T cells are CD95+ and CD45RA−. In some embodiments, at least 50% of the population of immune cells are CD95+ and CD45RA−. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD95+ and CD45RA−. In some embodiments, wherein at most 50% of the population of immune cells are CD95− and CD45RA+. In some embodiments, wherein at most 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD95− and CD45RA+.

In some embodiments, the CM T cells are CD95+, CCR7+ and CD45RA−. In some embodiments, at least 50% of the population of immune cells are CD95+, CCR7+ and CD45RA−. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD95+, CCR7+ and CD45RA−. In some embodiments, at most 50% of the population of immune cells are CD95−, CCR7− and CD45RA+. In some embodiments, at most 5%, 10%, 15%, 20%, 25%, 30%, 35%, 40%, 45%, 50%, 55%, 61%, 65%, 70%, 75%, 80%, 85%, 90%, 95%, or 99% of the population of immune cells are CD95−, CCR7− and CD45RA+.

In some embodiments, the CM T cells are CD38+. In some embodiments, at least 65% of the population of immune cells are CD38+. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD38+. In some embodiments, at most 35% of the population of immune cells are CD38−. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD38−.

In some embodiments, the CM T cells of the population of immune cells are CD25+. In some embodiments, at least 70% of the population of immune cells are CD25+. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD25+. In some embodiments, at most 30% of the population of immune cells are CD25−. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD25−.

In some embodiments, the CM T cells of the population of immune cells are CD38+ and CD25+. In some embodiments, at least 65% of the population of immune cells are CD38+ and CD25+. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD38+ and CD25+. In some embodiments, at most 20% of the population of immune cells are CD38− and CD25−. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD38− and CD25−.

In some embodiments, the CM T cells of the population of immune cells are PD-1+. In some embodiments, at least 50% of the population of immune cells are PD-1+. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the population of immune cells are PD-1+. In some embodiments, at most 50% of the population of immune cells are PD-1−. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the population of immune cells are PD-1−.

In some embodiments, the CM T cells of the population of immune cells are TIM-3+. In some embodiments, at least 42% of the population of immune cells are TIM-3+. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the population of immune cells are TIM-3+. In some embodiments, at most 10% of the population of immune cells are TIM-3−. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the population of immune cells are TIM-3−.

In some embodiments, the CM T cells of the population of immune cells are PD-1+ and TIM-3+. In some embodiments, at least 50% of the population of immune cells are PD-1+ and TIM-3+. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the population of immune cells are PD-1+ and TIM-3+. In some embodiments, at most 7% of the population of immune cells are PD-1− and TIM-3−. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the population of immune cells are PD-1− and TIM-3−.

In some embodiments, the CM T cells of the population of immune cells are IFNγ+. In some embodiments, at least 35% of the population of immune cells are IFNγ+. In some embodiments, at least 5%, 10%, 15%, 20%, 25%, 30%, 35%, 40%, 45%, 50%, 55%, 60%, 65%, 70%, 75%, 80%, 85%, 90%, 95%, or 99%/o of the population of immune cells are IFNγ+. In some embodiments, at most 65% of the population of immune cells are IFNγ−. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the population of immune cells are IFNγ−.

In some embodiments, the CM T cells of the population of immune cells are TNFα+. In some embodiments, at least 14% of the population of immune cells are TNFα+. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the population of immune cells are TNFα+. In some embodiments, at most 86% of the population of immune cells are TNFα−. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the population of immune cells are TNFα−.

In some embodiments, the CM T cells of the population of immune cells are IFNγ+ and TNFα+. In some embodiments, at least 10% of the population of immune cells are IFNγ+ and TNFα+. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the population of immune cells are IFNγ+ and TNFα+. In some embodiments, at most 52% of the population of immune cells are IFNγ− and TNFα−. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the population of immune cells are IFNγ− and TNFα−.

In some embodiments, the CM T cells of the population of immune cells are CD62L+. In some embodiments, at least 91% of the population of immune cells are CD62L+. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD62L+. In some embodiments, at most 9% of the population of immune cells are CD62L−. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD62L−.

In some embodiments, the CM T cells of the population of immune cells are CD44+. In some embodiments, at least 88% of the population of immune cells are CD44+. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD44+. In some embodiments, at most 12% of the population of immune cells are CD44−. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD44−.

In some embodiments, the CM T cells of the population of immune cells are CD62L+ and CD44+. In some embodiments, at least 15% of the population of immune cells are CD62L+ and CD44+. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD62L+ and CD44+. In some embodiments, at most 4% of the population of immune cells are CD62L− and CD44−. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the population of immune cells are CD62L− and CD44−.

In some embodiments, at least 35% of the cells in the population of immune cells are CD8+. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the cells in the population of immune cells are CD8+. In some embodiments, at most 65% of the cells in the population of immune cells are CD8−. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the cells in the population of immune cells are CD8−. In some embodiments, at least 65% of the cells in the population of immune cells are CD4+. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the cells in the population of immune cells are CD4+. In some embodiments, wherein at most 35% of the cells in the population of immune cells are CD4−. In some embodiments, wherein at most 5%, 10%, 15%, 20%, 25%, 30%, 35%, 40%, 45%, 50%, 55%, 60%, 65%, 70%, 75%, 80%, 85%, 90%, 95%, or 99% of the cells in the population of immune cells are CD4−.

In some embodiments, the CM T cells comprise CD4+ and/or CD8+ T cells. In some embodiments, the CM T cells comprise CD4+ T cells. In some embodiments, the CM T cells comprise CD8+ T cells. In some embodiments, at least 30% of the CM T cells are CD8+. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the CM T cells are CD8+. In some embodiments, at most 70% of the CM T cells are CD8−. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the CM T cells are CD8−. In some embodiments, at least 50% of the CM T cells are CD4+. In some embodiments, at least 5%, 10%, 15%, 20%, 25%, 30%, 35%, 40%, 45%, 50%, 55%, 60%, 65%, 70%, 75%, 80%, 85%, 90%, 95%, or 99% of the CM T cells are CD4+. In some embodiments, at most 50% of the CM T cells are CD4−. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the CM T cells are CD4−.

In some embodiments, at most 35% of the cells in the population of immune cells are naive T cells. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the cells in the population of immune cells are naive T cells. In some embodiments, at most 25% of the cells in the population of immune cells are EM T cells. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the cells in the population of immune cells are EM T cells. In some embodiments, at most 15% of the cells in the population of immune cells are T_{EMRA} T cells. In some embodiments, at most 5%, 10%, 15%, 20%, 25%, 30%, 35%, 40%, 45%, 50%, 55%, 60%, 65%, 70%, 75%, 80%, 85%, 90%, 95%, or 99% of the cells in the population of immune cells are T_{EMRA} T cells.

In some embodiments, the population of immune cells is derived from a biological sample from a subject or a peripheral blood mononuclear cell (PBMC) sample from a subject. In some embodiments, the CM T cells in the population of immune cells are derived from memory T cells in a biological sample from a subject, effector memory (EM) T cells in a biological sample from a subject, effector memory cells re-expressing CD45RA+ (T_{EMRA}) T cells in a biological sample from a subject, or EM T cells and Tre. T cells in a biological sample from a subject.

In some embodiments, the population of cells is an expanded population of immune cells from a cell population expanded in the presence of a molecule that binds to a TCRβV. In some embodiments, the percentage of CM T cells in the population of cells is higher than the percentage of CM T cells in a population of immune cells from the cell population expanded in the presence of a molecule that binds to a CD3. In some embodiments, the percentage of EM T cells in the population of immune cells is lower than the percentage of EM T cells in a population of cells from the cell population expanded in the presence of a CD3 binder. In some embodiments, the population of immune cells is a TCRβV binder-expanded population of cells. In some embodiments, the percentage of CM T cells in the TCRβV binder-expanded population of cells is higher than the percentage of CM T cells in a CD3 binder-expanded population of cells. In some embodiments, the percentage of EM T cells in the TCRβV binder-expanded population of cells is lower than the percentage of EM T cells in a CD3 binder-expanded population of cells.

Described herein is a composition comprising: a population of cells, wherein at least 50% of the population of cells are CM T cells, and a molecule that binds to a T cell receptor beta variable region (TCRβV). Also described herein is a cell culture comprising: a population of cells, wherein at least 50% of the population of cells are CM T cells, and a molecule that binds to a T cell receptor beta variable region (TCRβV). In some embodiments, the composition further comprises culture media. In some embodiments, the composition further comprises a growth factor or a cytokine. In some embodiments, the composition is within a container. In some embodiments, the container is a flask, a dish, a tube, a bag or a well.

As described herein, central memory (CM) T cell are T cells that express CCR7 but not CD45RA. CM T cells may also express CD45RO and/or CD62L.

As described herein, effector memory (EM) T cells that express CD45RO but not CCR7. EM T cells may also express CD44.

As described herein, TEMRA T cells are EM T cells that re-expresses CD45RA.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In some embodiments, the individual is a human. In some embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In some embodiments, a fetus is treated in utero, e.g., by administering the multifunctional or multispecific molecules or the compositions as described herein to the fetus directly or indirectly (e.g., via the mother).

Suitable routes for administration of the multifunctional or multispecific molecules or the compositions as described herein may vary depending on cell type to which delivery of the multifunctional or multispecific molecules or the compositions is desired. The multifunctional or multispecific molecules or the compositions as described herein may be administered to patients parenterally, for example, by intrathecal injection, intracerebroventricular injection, intraperitoneal injection, intramuscular injection, subcutaneous injection, or intravenous injection.

In some embodiments, the multifunctional or multispecific molecules or the compositions as described herein are administered with one or more agents capable of promoting penetration of the subject the multifunctional or multispecific molecules or the compositions as described herein across the blood-brain barrier by any method known in the art. For example, delivery of agents by administration of an adenovirus vector to motor neurons in muscle tissue is described in U.S. Pat. No. 6,632,427. “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523. “Adenovirus vectors for the transfer of foreign genes into cells of the central nervous system particularly in brain.” incorporated herein by reference.

In some embodiments, the multifunctional or multispecific molecules or the compositions as described herein are linked or conjugated with agents that provide desirable pharmaceutical or pharmacodynamic properties. In some embodiments, the multifunctional or multispecific molecules or the compositions as described herein are coupled to a substance, known in the art to promote penetration or transport across the blood-brain barrier, e.g., an antibody to the transferrin receptor. In some embodiments, the multifunctional or multispecific molecules or the compositions as described herein are linked with a viral vector.

In some embodiments, subjects treated using the methods and compositions are evaluated for improvement in condition using any methods known and described in the art.

The terms “treat,” “treating”, and “treatment,” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly, a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The term “prophylaxis” is used herein to refer to a measure or measures taken for the prevention or partial prevention of a disease or condition. In some embodiments, the terms “condition.” “disease,” or “disorder,” as used herein, are interchangeable.

By “treating or preventing a disease or a disorder” is meant ameliorating any of the conditions or signs or symptoms associated with the disorder before or after it has occurred. As compared with an equivalent untreated control, such reduction or degree of prevention is at least 3%, 5%, 10%, 20%, 40%, 50%, 60%, 80%, 90%, 95%, or 100% as measured by any standard technique. A patient who is being treated for a disease or a disorder, is one who a medical practitioner has diagnosed as having such a condition. Diagnosis may be by any suitable means. Diagnosis and monitoring may involve, for example, detecting the presence of pathological cells in a biological sample (e.g., tissue biopsy, blood test, or urine test), detecting the level of a surrogate marker of the disorder in a biological sample, or detecting symptoms associated with the disorder. A patient in whom the development of a disorder is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

Described herein is a method of vaccinating a subject comprising administering to a subject a pharmaceutical composition described in the present application. In some embodiments, the subject has been previously administered an antigen or a polynucleotide encoding an antigen. In some embodiments, the subject has a disease or condition. In some embodiments, the T cells specific to an antigen associated with the disease or condition are elicited in the subject. In some embodiments, B cells specific to an antigen associated with the disease or condition are elicited in the subject. In some embodiments, antigen-presenting cells specific to an antigen associated with the disease or condition are elicited in the subject. In some embodiments, natural killer cells targeting an antigen associated with the disease or condition are elicited in the subject. In some embodiments, macrophages targeting an antigen associated with the disease or condition are elicited in the subject. In some embodiments, neutrophils targeting an antigen associated with the disease or condition are elicited in the subject. In some embodiments, further comprising administering to the subject an antigen or a polynucleotide encoding an antigen after administration of the pharmaceutical composition described in the present application.

### Methods of Cancer Treatment

Described herein, in certain embodiments, is a method of treating a condition or disease in a subject in need therefor comprising administering to the subject a therapeutically effective amount of the multifunctional polypeptide molecule as described herein, the nucleic acid molecules as described herein, the vector as described herein, the cell as described herein, the pharmaceutical composition as described herein, or a combination thereof, wherein the administering is effective to treat the condition or disease in the subject.

In some embodiments, the condition or disease is cancer. In some embodiments, the cancer is a solid tumor, a hematological cancer, a metastatic cancer, a soft tissue tumor, or a combination thereof. In some embodiments, the cancer is the solid tumor, and wherein the solid tumor is selected from the group consisting of melanoma, pancreatic cancer, breast cancer, colorectal cancer, lung cancer, skin cancer, ovarian cancer, liver cancer, and a combination thereof. In some embodiments, the cancer is the hematological cancer, and wherein the hematological cancer is selected from the group consisting of Hodgkin's lymphoma, Non-Hodgkin's lymphoma, acute myeloid leukemia (AML), chronic myeloid leukemia, myelodysplastic syndrome, multiple myeloma, T-cell lymphoma, acute lymphocytic leukemia, and a combination thereof. In some embodiments, the Non-Hodgkin's lymphoma is selected from the group consisting of B cell lymphoma, diffuse large B cell lymphoma (DLBCL), follicular lymphoma, chronic lymphocytic leukemia (B-CLL), mantle cell lymphoma, marginal zone B-cell lymphoma, Burkitt lymphoma, lymphoplasmacy tic lymphoma, hairy cell leukemia, and a combination thereof. In some embodiments, the T-cell lymphoma is peripheral T-cell lymphoma.

In some embodiments, the cancer is characterized by a cancer antigen present on the cancer. In some embodiments, the cancer antigen is a tumor antigen, a stromal antigen, or a hematological antigen. In some embodiments, the cancer antigen is selected from the group consisting of BCMA, CD19, CD20, CD22, FcRH5, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, a actinin-4, TRP1/gp75, TRP2, gp100, Melan-A/MART1, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, gpA33, GD3, GM2, VEGFR, Intergrins, carbohydrates, IGF1R, EPHA3, TRAILR1, TRAILR2, RANKL, FAP, TGF-beta, hyaluronic acid, collagen, tenascin C, and tenascin W.

Methods described herein include treating a cancer in a subject by using multispecific or multifunctional molecules as described herein, e.g., using a pharmaceutical composition described herein. Also provided are methods for reducing or ameliorating a symptom of a cancer in a subject, as well as methods for inhibiting the growth of a cancer and/or killing one or more cancer cells. In some embodiments, the methods described herein decrease the size of a tumor and/or decrease the number of cancer cells in a subject administered with a described herein or a pharmaceutical composition described herein.

Described herein are methods of treating a subject having a cancer comprising acquiring a status of one or more TCRBV molecules in a subject. In some embodiments, a higher, e.g., increased, level or activity of one or more TCRβV molecules in a subject, e.g., in a sample from a subject, is indicative of a bias, e.g., a preferential expansion, e.g., clonal expansion, of T cells expressing said one or more TCRβV molecules in the subject.

Without wishing to be bound by theory, it is believed that a biased T cell population, e.g., a T cell population expressing a TCRβV molecule, is antigen-specific for a disease antigen, e.g., a cancer antigen (Wang C Y. et al., Int J Oncol. (2016) 48(6):2247-56). In some embodiments, the cancer antigen comprises a cancer associated antigen or a neoantigen. In some embodiments, a subject having a cancer, e.g., as described herein, has a higher, e.g., increased, level or activity of one or more TCRβV molecules associated with the cancer. In some embodiments, the TCRβV molecule is associated with, e.g., recognizes, a cancer antigen, e.g., a cancer associated antigen or a neoantigen.

Accordingly, as described herein are methods of expanding an immune effector cell population obtained from a subject, comprising acquiring a status of one or more TCRβV molecules in a sample from the subject, comprising contacting said immune effector cell population with an anti-TCRβV antibody molecule as described herein, e.g., an anti-TCRβV antibody molecule that binds to the same TCRβV molecule that is higher, e.g., increased in the immune effector cell population in the sample from the subject. In some embodiments, contacting the population of immune effector cells (e.g., comprising T cells that express one or more TCRβV molecules) with an anti-TCRβV molecule results in expansion of the population of immune effector cells expressing one or more TCRβV molecules. In some embodiments, the expanded population, or a portion thereof, is administered to the subject (e.g., same subject from whom the immune effector cell population was obtained), to treat the cancer. In some embodiments, the expanded population, or a portion thereof, is administered to a different subject (e.g., not the same subject from whom the immune effector cell population was obtained), to treat the cancer.

Also described herein are methods of treating a subject having a cancer, comprising: acquiring a status of one or more TCRβV molecules in a sample from the subject, and determining whether the one or more TCRβV molecules is higher, e.g., increased, in a sample from the subject compared to a reference value, wherein responsive to said determination, administering to the subject an effective amount of an anti-TCRβV antibody molecule, e.g., an agonistic anti-TCRβV antibody molecule, e.g., as described herein.

In some embodiments, the subject has B-CLL. In some embodiments, a subject having B-CLL has a higher, e.g., increased, level or activity of one or more TCRβV molecules, e.g., one or more TCRβV molecules comprising: (i) TCRβ V6 subfamily comprising, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01; (ii) TCRβ V5 subfamily comprising TCRβ V5-6*01, TCRβ V54*01, or TCRβ V5-8*01; (iii) TCRβ V3 subfamily comprising TCRβ V3-1*01; (iv) TCRβ V2 subfamily comprising TCRβ V2*01; or (v) TCRβ V19 subfamily comprising TCRβ V19*01, or TCRβ V19*02.

In some embodiments, a subject having B-CLL has a higher, e.g., increased, level or activity of a TCRβ V6 subfamily comprising, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V6 subfamily. In some embodiments, administration of the multifunctional polypeptide molecule as described herein results in expansion of immune cells expressing one or more members of the TCRβ V6 subfamily.

In some embodiments, a subject having B-CLL has a higher, e.g., increased, level or activity of a TCRβ V5 subfamily comprising TCRβ V5-6*01, TCRβ V5-4*01, or TCRβ V5-8*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V5 subfamily. In some embodiments, administration of the multifunctional polypeptide molecule as described herein results in expansion of immune cells expressing one or more members of the TCRβ V5 subfamily.

In some embodiments, a subject having B-CLL has a higher, e.g., increased, level or activity of a TCRβ V3 subfamily comprising TCRβ V3-1*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V3 subfamily. In some embodiments, administration of the multifunctional polypeptide molecule as described herein results in expansion of immune cells expressing one or more members of the TCRβ V3 subfamily.

In some embodiments, a subject having B-CLL has a higher, e.g., increased, level or activity of a TCRβ V2 subfamily comprising TCRβ V2*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V2 subfamily. In some embodiments, administration of the multifunctional polypeptide molecule as described herein results in expansion of immune cells expressing one or more members of the TCRβ V2 subfamily.

In some embodiments, a subject having B-CLL has a higher, e.g., increased, level or activity of a TCRβ V19 subfamily comprising TCRβ V19*01, or TCRβ V19*02. In some embodiments, the subject is administered the multifunctional polypeptide molecule as described herein comprising an anti-TCRβV molecule (e.g., an agonistic anti-TCRBV molecule as described herein) that binds to one or more members of the TCRβ V19 subfamily. In some embodiments, administration of the multifunctional polypeptide molecule as described herein results in expansion of immune cells expressing one or more members of the TCRβ V19 subfamily.

In some embodiments, the subject has melanoma. In some embodiments, a subject having melanoma has a higher, e.g., increased, level or activity of one or more TCRβV molecules, e.g., one or more TCRβV molecules comprising the TCRβ V6 subfamily comprising, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V6 subfamily. In some embodiments, administration of the multifunctional polypeptide molecule as described herein results in expansion of immune cells expressing one or more members of the TCRβ V6 subfamily.

In some embodiments, the subject has DLBCL (diffuse large B-cell lymphoma). In some embodiments, a subject having DLBCL has a higher, e.g., increased, level or activity of one or more TCRβV molecules, e.g., one or more TCRβV molecules comprising: (i) TCRβ V13 subfamily comprising TCRβ V13*01; (ii) TCRβ V3 subfamily comprising TCRβ V3-1*01; or (iii) TCRβ V23 subfamily.

In some embodiments, a subject having DLBCL has a higher, e.g., increased, level or activity of a TCRβ V13 subfamily comprising TCRβ V13*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V13 subfamily. In some embodiments, administration of the multifunctional polypeptide molecule as described herein results in expansion of immune cells expressing one or more members of the TCRβ V13 subfamily.

In some embodiments, a subject having DLBCL has a higher, e.g., increased, level or activity of a TCRβ V3 subfamily comprising TCRβ V3-1*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V3 subfamily. In some embodiments, administration of the multifunctional polypeptide molecule as described herein results in expansion of immune cells expressing one or more members of the TCRβ V3 subfamily.

In some embodiments, a subject having DLBCL has a higher, e.g., increased, level or activity of a TCRβ V23 subfamily. In some embodiments, the subject is administered the multifunctional polypeptide molecule as described herein comprising an anti-TCRβV molecule (e.g., an agonistic anti-TCRβV molecule as described herein) that binds to one or more members of the TCRβ V23 subfamily. In some embodiments, administration of the multifunctional polypeptide molecule as described herein results in expansion of immune cells expressing one or more members of the TCRβ V23 subfamily.

In some embodiments, the subject has CRC (colorectal cancer). In some embodiments, a subject having CRC has a higher, e.g., increased, level or activity of one or more TCRβV molecules, e.g., one or more TCRβV molecules comprising: (i) TCRβ V19 subfamily comprising TCRβ V19*01, or TCRβ V19*02. (ii) TCRβ V12 subfamily comprising TCRβ V12-4*01, TCRβ V12-3*01, or TCRβ V12-5*01; (iii) TCRβ V16 subfamily comprising TCRβ V16*01: or (iv) TCRβ V21 subfamily.

In some embodiments, a subject having CRC has a higher, e.g., increased, level or activity of a TCRβ V19 subfamily comprising TCRβ V19*01, or TCRβ V19*02. In some embodiments, the subject is administered the multifunctional polypeptide molecule as described herein comprising an anti-TCRβV molecule (e.g., an agonistic anti-TCRβV molecule as described herein) that binds to one or more members of the TCRβ V19 subfamily. In some embodiments, administration of the multifunctional poly peptide molecule as described herein results in expansion of immune cells expressing one or more members of the TCRβ V19 subfamily.

In some embodiments, a subject having CRC has a higher, e.g., increased, level or activity of a TCRβ V12 subfamily comprising TCRβ V12-4*01, TCRβ V12-3*01, or TCRβ V12-5*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V12 subfamily. In some embodiments, administration of the multifunctional polypeptide molecule as described herein results in expansion of immune cells expressing one or more members of the TCRβ V12 subfamily.

In some embodiments, a subject having CRC has a higher, e.g., increased, level or activity of a TCRβ V16 subfamily comprising TCRβ V16*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V16 subfamily. In some embodiments, administration of the multifunctional polypeptide molecule as described herein results in expansion of immune cells expressing one or more members of the TCRβ V16 subfamily.

In some embodiments, a subject having CRC has a higher, e.g., increased, level or activity of a TCRβ V21 subfamily. In some embodiments, the subject is administered the multifunctional polypeptide molecule as described herein comprising an anti-TCRβV molecule (e.g., an agonistic anti-TCRβV molecule as described herein) that binds to one or more members of the TCRβ V21 subfamily. In some embodiments, administration of the multifunctional polypeptide molecule as described herein results in expansion of immune cells expressing one or more members of the TCRβ V21 subfamily.

In some embodiments, acquiring a value for the status, e.g., presence, level and/or activity, of one or more TCRβV molecules comprises acquiring a measure of the T cell receptor (TCR) repertoire of a sample. In some embodiments, the value comprises a measure of the clonotype of a population of T cells in the sample.

In some embodiments, a value for the status of one or more TCRβV molecules is obtained, e.g., measured, using an assay described in Wang C Y, et al., Int J Oncol. (2016) 48(6):2247-56, the entire contents of which are hereby incorporated by reference.

In some embodiments, a value for the status of one or more TCRβV molecules is obtained, e.g., measured, using flow cytometry.

### Combination Therapies

In some embodiments, the method as described herein further comprises administering a second therapeutic agent or therapy to the subject.

In some embodiments, the second therapeutic agent or therapy comprises a chemotherapeutic agent, a biologic agent, a hormonal therapy, radiation, or surgery.

In some embodiments, the second therapeutic agent or therapy is administered in combination with the multifunctional polypeptide molecule as described herein, the nucleic acid molecules as described herein, the vector as described herein, the cell as described herein, the pharmaceutical composition as described herein, sequentially, simultaneously, or concurrently.

The multispecific or multifunctional molecules as described herein can be used in combination with a second therapeutic agent or procedure.

In some embodiments, the multispecific or multifunctional molecules as described herein and the second therapeutic agent or procedure are administered/performed after a subject has been diagnosed with a cancer, e.g., before the cancer has been eliminated from the subject. In some embodiments, the multispecific or multifunctional molecules as described herein and the second therapeutic agent or procedure are administered/performed simultaneously or concurrently. For example, the delivery of one treatment is still occurring when the delivery of the second commences, e.g., there is an overlap in administration of the treatments. In other embodiments, the multispecific or multifunctional molecules as described herein and the second therapeutic agent or procedure are administered/performed sequentially. For example, the delivery of one treatment ceases before the delivery of the other treatment begins.

In some embodiments, combination therapy can lead to more effective treatment than monotherapy with either agent alone. In some embodiments, the combination of the first and second treatment is more effective (e.g., leads to a greater reduction in symptoms and/or cancer cells) than the first or second treatment alone. In some embodiments, the combination therapy permits use of a lower dose of the first or the second treatment compared to the dose of the first or second treatment normally required to achieve similar effects when administered as a monotherapy. In some embodiments, the combination therapy has a partially additive effect, wholly additive effect, or greater than additive effect.

In some embodiments, the anti-TCRBV antibody, multispecific or multifunctional molecule is administered in combination with a therapy, e.g., a cancer therapy (e.g., one or more of anti-cancer agents, immunotherapy, photodynamic therapy (PDT), surgery and/or radiation). The terms “chemotherapeutic,” “chemotherapeutic agent,” and “anti-cancer agent” are used interchangeably herein. The administration of the multispecific or multifunctional molecule and the therapy, e.g., the cancer therapy, can be sequential (with or without overlap) or simultaneous. Administration of the anti-TCRBV antibody, multispecific or multifunctional molecule can be continuous or intermittent during the course of therapy (e.g., cancer therapy). Certain therapies described herein can be used to treat cancers and non-cancerous diseases. For example, PDT efficacy can be enhanced in cancerous and non-cancerous conditions (e.g., tuberculosis) using the methods and compositions described herein (reviewed in, e.g., Agostinis, P. et al. (2011) CA Cancer J. Clin. 61:250-281).

Methods described herein include treating a cancer in a subject by using the multispecific or multifunctional molecules as described herein, e.g., using a pharmaceutical composition as described herein. Also provided are methods for reducing or ameliorating a symptom of a cancer in a subject, as well as methods for inhibiting the growth of a cancer and/or killing one or more cancer cells. In some embodiments, the methods described herein decrease the size of a tumor and/or decrease the number of cancer cells in a subject administered with a described herein or a pharmaceutical composition described herein.

In some embodiments, the cancer is a hematological cancer. In some embodiments, the hematological cancer is a leukemia or a lymphoma. As used herein, a “hematologic cancer” refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocy tic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm. Langerhans cell histiocytosis, multiple myeloma/plasma cell neoplasm, myelodysplastic syndrome, or myelodysplastic/myeloproliferative neoplasm.

In some embodiments, the cancer is a myeloproliferative neoplasm, e.g., primary or idiopathic myelofibrosis (MF), essential thrombocytosis (ET), polycythemia vera (PV), or chronic myelogenous leukemia (CML). In some embodiments, the cancer is myelofibrosis. In some embodiments, the subject has myelofibrosis. In some embodiments, the subject has a calreticulin mutation, e.g., a calreticulin mutation as described herein. In some embodiments, the subject does not have the JAK2-V617F mutation. In some embodiments, the subject has the JAK2-V617F mutation. In some embodiments, the subject has a MPL mutation. In some embodiments, the subject does not have a MPL mutation.

In some embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In some embodiments, the cancer is acute lymphoblastic leukemia, acute lymphocytic leukemia, acute myelogenous leukemia, aplastic anemia, chronic myelogenous leukemia, desmoplastic small round cell tumor, Ewing's sarcoma, Hodgkin's disease, multiple myeloma, myelodysplasia, Non-Hodgkin's lymphoma, paroxysmal nocturnal hemoglobinuria, radiation poisoning, chronic lymphocytic leukemia, AL amyloidosis, essential thrombocytosis, polycythemia vera, severe aplastic anemia, neuroblastoma, breast tumors, ovarian tumors, renal cell carcinoma, autoimmune disorders, such as systemic sclerosis, osteopetrosis, inherited metabolic disorders, juvenile chronic arthritis, adrenoleukodystrophy, amegakaryocytic thrombocytopenia, sickle cell disease, severe congenital immunodeficiency, Griscelli syndrome type II, Hurler syndrome, Kostmann syndrome, Krabbe disease, metachromatic leukodystrophy, thalassemia, hemophagocytic lymphohistiocytosis, and Wiskott-Aldrich syndrome, leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma. B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple. Phyllodes Tumors. Lobular Carcinoma. Ductal Carcinoma, cancer of the pancreatic stellate cells, cancer of the hepatic stellate cells, or prostate cancer. In some embodiments, the cancer is a solid tumor. In some embodiments, the cancer is hematological.

In some embodiments, the multispecific or multifunctional molecules as described herein (or pharmaceutical composition as described herein) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or multispecific or multifunctional molecules) to be administered can be determined by a physician with consideration of individual differences in tumor size, extent of infection or metastasis, age, weight, and condition of the subject. In some embodiments, the pharmaceutical composition described herein can be administered at a dosage of 10⁴ to 10⁹ cells/kg body weight, e.g., 10⁵ to 10⁶ cells/kg body weight, including all integer values within those ranges. In some embodiments, the pharmaceutical composition described herein can be administered multiple times at these dosages. In some embodiments, the pharmaceutical composition described herein can be administered using infusion techniques described in immunotherapy (see, e.g., Rosenberg et al., New Eng. J. of Med. 319:1676, 1988).

In some embodiments, the multispecific or multifunctional molecules as described herein or the pharmaceutical composition as described herein is administered to the subject parentally. In some embodiments, the cells are administered to the subject intravenously, subcutaneously, intratumorally, intranodally, intramuscularly, intradermally, or intraperitoneally. In some embodiments, the cells are administered, e.g., injected, directly into a tumor or lymph node. In some embodiments, the cells are administered as an infusion (e.g., as described in Rosenberg et al., New Eng. J. of Med. 319:1676, 1988) or an intravenous push. In some embodiments, the cells are administered as an injectable depot formulation.

In some embodiments, the subject is a mammal. In some embodiments, the subject is a human, monkey, pig, dog, cat, cow, sheep, goat, rabbit, rat, or mouse. In some embodiments, the subject is a human. In some embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In some embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Anti-Cancer Therapies

In other embodiments, the multispecific or multifunctional molecules as described herein is administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®), altretamine (hexamethylmelamine, HMM, HEXALEN®), amethopterin (methotrexate, methotrexate sodium, MTX, TREXALL™, RHEUMATREX®), amifostine (ETHYOL®), arabinosylcytosine (Ara-C, cytarabine, CYTOSAR-U®)), arsenic trioxide (TRISENOX®), asparaginase (Erwinia L-asparaginase, L-asparaginase, ELSPAR®, KIDROLASE®), BCNU (carmustine, BiCNU®), bendamustine (TREANDA®), bexarotene (TARGRETIN®), bleomycin (BLENOXANE®), busulfan (BUSULFEX®), MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant, GLIADEL®, wafer), CCI-779 (temsirolimus, TORISEL®), CCNU (lomustine, CeeNU), CDDP (cisplatin, PLATINOL®, PLATINOL-AQ®), chlorambucil (leukeran), cyclophosphamide (CYTOXAN®). NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomycin-C, MTC, MUTAMYCIN®), nelarabine (ARRANON®), oxaliplatin (ELOXATIN™), paclitaxel (TAXOL®, ONXALT™), pegaspargase (PEG-L-asparaginase, ONCOSPAR®). PEMETREXED (ALIMTA®), pentostatin (NIPENT®), procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN), vinorelbine (vinorelbine tartrate, NAVELBINE®), and vorinostat (ZOLINZA®).

In another embodiment, the multispecific or multifunctional molecules as described herein is administered in conjunction with a biologic. Biologics useful in the treatment of cancers are known in the art and a binding molecule as described herein may be administered, for example, in conjunction with such known biologics. For example, the FDA has approved the following biologics for the treatment of breast cancer: HERCEPTIN® (trastuzumab, Genentech Inc., South San Francisco, Calif.; a humanized monoclonal antibody that has anti-tumor activity in HER2-positive breast cancer); FASLODEX® (fulvestrant, AstraZeneca Pharmaceuticals. LP. Wilmington, Del.; an estrogen-receptor antagonist used to treat breast cancer): ARIMIDEX® (anastrozole, AstraZeneca Pharmaceuticals. LP: a nonsteroidal aromatase inhibitor which blocks aromatase, an enzyme needed to make estrogen); Aromasin® (exemestane, Pfizer Inc., New York, N.Y.: an irreversible, steroidal aromatase inactivator used in the treatment of breast cancer): FEMARA® (letrozole, Novartis Pharmaceuticals, East Hanover, N.J.: a nonsteroidal aromatase inhibitor approved by the FDA to treat breast cancer); and NOLVADEX® (tamoxifen, AstraZeneca Pharmaceuticals. LP; a nonsteroidal antiestrogen approved by the FDA to treat breast cancer). Other biologics with which the binding molecules as described herein may be combined include: AVASTIN® (bevacizumab. Genentech Inc.; the first FDA-approved therapy designed to inhibit angiogenesis); and ZEVALIN® (ibritumomab tiuxetan. Biogen Idec, Cambridge, Mass.; a radiolabeled monoclonal antibody currently approved for the treatment of B-cell lymphomas).

In addition, the FDA has approved the following biologics for the treatment of colorectal cancer: AVASTIN®; ERBITUX® (cetuximab, ImClone Systems Inc., New York, N.Y., and Bristol-Myers Squibb, New York, N.Y.; is a monoclonal antibody directed against the epidermal growth factor receptor (EGFR)); GLEEVEC® (imatinib mesylate: a protein kinase inhibitor); and ERGAMISOL® (levamisole hydrochloride. Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For the treatment of lung cancer, exemplary biologics include TARCEVA® (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include VELCADE® (bortezomib, Millennium Pharmaceuticals. Cambridge Mass.; a proteasome inhibitor). Additional biologics include THALIDOMID® (thalidomide, Clegene Corporation, Warren, N.J.; an immunomodulatory agent and appears to have multiple actions, including the ability to inhibit the growth and survival of myeloma cells and anti-angiogenesis).

Additional exemplary cancer therapeutic antibodies include, but are not limited to, 3F8, abagovomab, adecatumumab, afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), bavituximab, bectumomab (LYMPHOSCAN®), belimumab (BENLYSTA®, LYMPHOSTAT-B®), besilesomab (SCINTIMUN®), bevacizunab (AVASTIN®), bivatuzumab mertansine, blinatunomab, brentuximab vedotin, cantuzumab mertansine, capromab pendetide (PROSTASCINT®), catumaxomab (REMOVAB®), CC49, cetuximab (C225, ERBITUX®), citatuzumab bogatox, cixutumumab, clivatuzumab tetraxetan, conatumumab, dacetuzumab, denosumab (PROLIA®), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®), girentuximab, glembatumumab vedotin, ibritumomab (ibritumomab tiuxetan, ZEVALIN®), igovomab (INDIMACIS-125®), intetumumab, inotuzumab ozogamicin, ipilimumab, iratumumab, labetuzumab (CEA-CIDE®), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®), THERALOC®), nofetumomab merpentan (VERLUMA®), ofatumumab (ARZERRA), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®, RITUXAN®), robatumumab, satumomab pendetide, sibrotuzumab, siltuximab, sontuzumab, tacatuzumab tetraxetan (AFP-CIDE®), taplitumomab paptox, tenatumomab, TGN1412, ticilimumab (tremelimumab), tigatuzumab, TNX-650, tositumomab (BEXXAR®), trastuzumab (HERCEPTIN®), tremelimumab, tucotuzumab celmoleukin, veltuzumab, volociximab, votumumab (HUMASPECT®), zalutumumab (HUMAX-EGFR®), and zanolimumab (HUMAX-CD4®).

In some embodiments, the multispecific or multifunctional molecules as described herein are administered in combination with a viral cancer therapeutic agent. Exemplary viral cancer therapeutic agents include, but not limited to, vaccinia virus (vvDD-CDSR), carcinoembryonic antigen-expressing measles virus, recombinant vaccinia virus (TK-deletion plus GM-CSF), Seneca Valley virus-001, Newcastle virus, coxsackie virus A21, GL-ONC1, EBNA1 C-terminal/LMP2 chimeric protein-expressing recombinant modified vaccinia Ankara vaccine, carcinoembryonic antigen-expressing measles virus, G207 oncolytic virus, modified vaccinia virus Ankara vaccine expressing p53, OncoVEX GM-CSF modified herpes-simplex 1 virus, fowlpox virus vaccine vector, recombinant vaccinia prostate-specific antigen vaccine, human papillomavirus 16/18 L1 virus-like particle/ASO4 vaccine, MVA-EBNA1/LMP2 Inj, vaccine, quadrivalent HPV vaccine, quadrivalent human papillomavirus (types 6, 11, 16, 18) recombinant vaccine (GARDASIL®), recombinant fowlpox-CEA(6D)/TRICOM vaccine; recombinant vaccinia-CEA(6D)-TRICOM vaccine, recombinant modified vaccinia Ankara-5T4 vaccine, recombinant fowlpox-TRICOM vaccine, oncolytic herpes virus NV1020, HPV L1 VLP vaccine V504, human papillomavirus bivalent (types 16 and 18) vaccine (CERVARIX(@), herpes simplex virus HF10, Ad5CMV-p53 gene, recombinant vaccinia DF3/MUC1 vaccine, recombinant vaccinia-MUC-1 vaccine, recombinant vaccinia-TRICOM vaccine, ALVAC MART-1 vaccine, replication-defective herpes simplex virus type I (HSV-1) vector expressing human Preproenkephalin (NP2), wild-type reovirus, reovirus type 3 Dearing (REOLYSIN®), oncolytic virus HSV1716, recombinant modified vaccinia Ankara (MVA)-based vaccine encoding Epstein-Barr virus target antigens, recombinant fowlpox-prostate specific antigen vaccine, recombinant vaccinia prostate-specific antigen vaccine, recombinant vaccinia-B7.1 vaccine, rAd-p53 gene, Ad5-delta24RGD, HPV vaccine 580299, JX-594 (thymidine kinase-deleted vaccinia virus plus GM-CSF). HPV-16/18 L1/ASO4, fowlpox virus vaccine vector, vaccinia-tyrosinase vaccine, MEDI-517 HPV-16/18 VLP AS04 vaccine, adenoviral vector containing the thymidine kinase of herpes simplex virus TK99UN, HspE7, FP253/Fludarabine, ALVAC(2) melanoma multi-antigen therapeutic vaccine, ALVAC-hB7.1, canarypox-hIL-12 melanoma vaccine, Ad-REIC/Dkk-3, rAd-IFN SCH 721015, TIL-Ad-INFg, Ad-ISF35, and coxsackievirus A21 (CVA21, CAVATAK®).

In some embodiments, the multispecific or multifunctional molecules as described herein are administered in combination with a nanopharmaceutical. Exemplary cancer nanopharmaceuticals include, but not limited to, ABRAXANE® (paclitaxel bound albumin nanoparticles), CRLX101 (CPT conjugated to a linear cyclodextrin-based polymer), CRLX288 (conjugating docetaxel to the biodegradable polymer poly (lactic-co-glycolic acid)), cytarabine liposomal (liposomal Ara-C, DEPOCYT®), daunorubicin liposomal (DAUNOXOME®), doxorubicin liposomal (DOXIL®, CAELYX®), encapsulated-daunorubicin citrate liposome (DAUNOXOME®), and PEG anti-VEGF aptamer (MACUGEN®).

In some embodiments, the multispecific or multifunctional molecules as described herein are administered in combination with paclitaxel or a paclitaxel formulation, e.g., TAXOL®, protein-bound paclitaxel (e.g., ABRAXANE®). Exemplary paclitaxel formulations include, but are not limited to, nanoparticle albumin-bound paclitaxel (ABRAXANE®, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP). ANG105 (Angiopep-2 bound to three molecules of paclitaxel, marketed by ImmunoGen), paclitaxel-EC-1 (paclitaxel bound to the erbB2-recognizing peptide EC-1: see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620).

Exemplary RNAi and antisense RNA agents for treating cancer include, but not limited to, CALAA-01, siG12D LODER (Local Drug EluteR), and ALN-VSP02.

Other cancer therapeutic agents include, but not limited to, cytokines (e.g., aldesleukin (IL-2, Interleukin-2, PROLEUKIN®), alpha Interferon (IFN-alpha, Interferon alfa, INTRON® A (Interferon alfa-2b), ROFERON-A® (Interferon alfa-2a)), Epoetin alfa (PROCRIT®), filgrastim (G-CSF, Granulocyte—Colony Stimulating Factor, NEUPOGEN®), GM-CSF (Granulocyte Macrophage Colony Stimulating Factor, sargramostim, LEUKINE™), IL-11 (Interleukin-11, oprelvekin, NEUMEGA®), Interferon alfa-2b (PEG conjugate) (PEG interferon, PEG-INTRONT™), and pegfilgrastim (NEULASTA™)), hormone therapy agents (e.g., aminoglutethimide (CYTADREN®), anastrozole (ARIMIDEX®), bicalutamide (CASODEX®), exemestane (AROMASIN®), fluoxymesterone (HALOTESTIN®), flutamide (EULEXIN®), fulvestrant (FASLODEX®), goserelin (ZOLADEX®), letrozole (FEMARA®), leuprolide (ELIGARD®, LUPRON®, LUPRON DEPOT®, VIADUR™), megestrol (megestrol acetate, MEGACE®), nilutamide (ANANDRON®, NILANDRON®), octreotide (octreotide acetate, SANDOSTATIN®, SANDOSTATIN LAR®), raloxifene (EVISTA®)), romiplostim (NPLATE®), tamoxifen (NOVALDEX®), and toremifene (FARESTON®)), phospholipase A2 inhibitors (e.g., anagrelide (AGRYLIN®)), biologic response modifiers (e.g., BCG (THERACYS®, TICE®), and Darbepoetin alfa (ARANESP®)), target therapy agents (e.g., bortezomib (VELCADE®), dasatinib (SPRYCEL®), denileukin diftitox (ONTAK®), erlotinib (TARCEVA®), everolimus (AFINITOR®), gefitinib (IRESSA®), imatinib mesylate (STI-571, GLEEVEC™), lapatinib (TYKERB®), sorafenib (NEXAVAR®), and SU11248 (sunitinib, SUTENT®)), immunomodulatory and antiangiogenic agents (e.g., CC-5013 (lenalidomide, REVLIMID®), and thalidomide (THALOMID®)), glucocorticosteroids (e.g., cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PRELONE®), and prednisone (DELTASONE®, LIQUID PRED®, METICORTEN®, ORASONE®)), and bisphosphonates (e.g., pamidronate (AREDIA®), and zoledronic acid (ZOMETA®)).

In some embodiments, the multispecific or multifunctional molecules as described herein are used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., an antibody against VEGF, a VEGF trap, a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-B inhibitor)), a RAF-1 inhibitor, a KIT inhibitor and a RET inhibitor. In some embodiments, the anti-cancer agent used in combination with the AHCM agent is selected from the group consisting of: axitinib (AG013736), bosutinib (SKI-606), cediranib (RECENTIN™, AZD2171), dasatinib (SPRYCEL®, BMS-354825), erlotinib (TARCEVA®), gefitinib (IRESSA)), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951 (tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride, PD173074, Sorafenib Tosylate (Bay 43-9006), SU 5402, TSU-68 (SU6668), vatalanib, XL880 (GSK 1363089, EXEL-2880). Selected tyrosine kinase inhibitors are chosen from sunitinib, erlotinib, gefitinib, or sorafenib. In some embodiments, the tyrosine kinase inhibitor is sunitinib.

In some embodiments, the multispecific or multifunctional molecules as described herein are administered in combination with one of more of: an anti-angiogenic agent, or a vascular targeting agent or a vascular disrupting agent. Exemplary anti-angiogenic agents include, but are not limited to, VEGF inhibitors (e.g., anti-VEGF antibodies (e.g., bevacizumab): VEGF receptor inhibitors (e.g., itraconazole); inhibitors of cell proliferatin and/or migration of endothelial cells (e.g., carboxyamidotriazole, TNP-470); inhibitors of angiogenesis stimulators (e.g., suramin), among others. A vascular-targeting agent (VTA) or vascular disrupting agent (VDA) is designed to damage the vasculature (blood vessels) of cancer tumors causing central necrosis (reviewed in, e.g., Thorpe, P. E. (2004) Clin. Cancer Res. Vol. 10:415-427). VTAs can be small-molecule. Exemplary small-molecule VTAs include, but are not limited to, microtubule destabilizing drugs (e.g., combretastatin A-4 disodium phosphate (CA4P), ZD6126, AVE8062, Oxi 4503); and vadimezan (ASA404).

### Immune Checkpoint Inhibitors

In other embodiments, methods described herein comprise use of an immune checkpoint inhibitor in combination with the multispecific or multifunctional molecules as described herein. The methods can be used in a therapeutic protocol in vivo.

In some embodiments, an immune checkpoint inhibitor inhibits a checkpoint molecule. Exemplary checkpoint molecules include but are not limited to CTLA4. PD1, PD-L1, PD-L2. TIM3, LAG3, CD160, 2B4, CD80, CD86, B7-H3 (CD276), B7-H4 (VTCN1), HVEM (TNFRSF14 or CD270), BTLA, KIR, MHC class I, MHC class II, GAL9, VISTA, BTLA, TIGIT, LAIR1, and A2aR. See, e.g., Pardoll. Nat. Rev. Cancer 12.4(2012):252-64, incorporated herein by reference.

In some embodiments, the immune checkpoint inhibitor is a PD-1 inhibitor, e.g., an anti-PD-1 antibody such as Nivolumab, Pembrolizumab or Pidilizumab. Nivolumab (also called MDX-1106, MDX-1106-04. ONO-4538, or BMS-936558) is a fully human IgG4 monoclonal antibody that specifically inhibits PD1. See, e.g., U.S. Pat. No. 8,008,449 and WO2006/121168. Pembrolizumab (also called Lambrolizumab, MK-3475, MK03475, SCH-900475 or KEYTRUDA®; Merck) is a humanized IgG4 monoclonal antibody that binds to PD-1. See, e.g., Hamid, O. et al. (2013) New Engl and Journal of Medicine 369 (2): 134-44, U.S. Pat. No. 8,354,509 and WO2009/114335. Pidilizumab (also called CT-011 or Cure Tech) is a humanized IgG1k monoclonal antibody that binds to PD1. See, e.g., WO2009/101611. In some embodiments, the inhibitor of PD-1 is an antibody molecule having a sequence substantially identical or similar thereto, e.g., a sequence at least 85%, 90%, 95% identical or higher to the sequence of Nivolumab, Pembrolizumab or Pidilizumab. Additional anti-PD1 antibodies, e.g., AMP 514 (Amplimmune), are described, e.g., in U.S. Pat. No. 8,609,089. US 2010028330, and/or US 20120114649.

In some embodiments, the PD-1 inhibitor is an immunoadhesin, e.g., an immunoadhesin comprising an extracellular/PD-1 binding portion of a PD-1 ligand (e.g., PD-L1 or PD-L2) that is fused to a constant region (e.g., an Fc region of an immunoglobulin). In some embodiments, the PD-1 inhibitor is AMP-224 (B7-DCIg, e.g., described in WO2011/066342and WO2010/027827), a PD-L2 Fc fusion soluble receptor that blocks the interaction between B7-H1 and PD-1.

In some embodiments, the immune checkpoint inhibitor is a PD-L1 inhibitor, e.g., an antibody molecule. In some embodiments, the PD-L1 inhibitor is YW243.55.S70, MPDL3280A, MEDI-4736, MSB-0010718C, or MDX-1105. In some embodiments, the anti-PD-L1 antibody is MSB0010718C (also called A09-246-2; Merck Serono), which is a monoclonal antibody that binds to PD-L1. Exemplary humanized anti-PD-L1 antibodies are described, e.g., in WO2013/079174. In some embodiments, the PD-L1 inhibitor is an anti-PD-L1 antibody, e.g., YW243.55.S70. The YW243.55.S70 antibody is described. e.g., in WO 2010/077634. In some embodiments, the PD-L1 inhibitor is MDX-1105 (also called BMS-936559), which is described, e.g., in WO2007/005874. In some embodiments, the PD-L1 inhibitor is MDPL3280A (Genentech/Roche), which is a human Fc-optimized IgG1 monoclonal antibody against PD-L1. See, e.g., U.S. Pat. No. 7,943,743 and U.S. Publication No.: 20120039906. In some embodiments, the inhibitor of PD-L1 is an antibody molecule having a sequence substantially identical or similar thereto, e.g., a sequence at least 85%, 90%, 95% identical or higher to the sequence of YW243.55.S70, MPDL3280A, MEDI-4736, MSB-0010718C, or MDX-1105.

In some embodiments, the immune checkpoint inhibitor is a PD-L2 inhibitor, e.g., AMP-224 (which is a PD-L2 Fc fusion soluble receptor that blocks the interaction between PD1 and B7-H1. See, e.g., WO2010/027827 and WO2011/066342.

In some embodiments, the immune checkpoint inhibitor is a LAG-3 inhibitor, e.g., an anti LAG-3 antibody molecule. In some embodiments, the anti-LAG-3 antibody is BMS-986016 (also called BMS986016; Bristol-Myers Squibb). BMS-986016 and other humanized anti-LAG-3 antibodies are described, e.g., in US 2011/0150892, WO2010/019570, and WO2014/008218.

In some embodiments, the immune checkpoint inhibitor is a TIM-3 inhibitor, e.g., anti-TIM3 antibody molecule, e.g., described in U.S. Pat. No. 8,552,156. WO 2011/155607, EP 2581113 and U.S. Publication No.: 2014/044728.

In some embodiments, the immune checkpoint inhibitor is a CTLA-4 inhibitor, e.g., anti-CTLA-4 antibody molecule. Exemplary anti-CTLA4 antibodies include Tremelimumab (IgG2 monoclonal antibody from Pfizer, formerly known as ticilimumab, CP-675,206); and Ipilimumab (also called MDX-010. CAS No. 477202-00-9). Other exemplary anti-CTLA-4 antibodies are described, e.g., in U.S. Pat. No. 5,811,097.

### Method of Expanding Cells

Any of the compositions and methods described herein can be used to expand an immune cell population. An immune cell provided herein includes an immune cell derived from a hematopoietic stem cell or an immune cell derived from a non-hematopoietic stem cell, e.g., by differentiation or de-differentiation.

An immune cell includes a hematopoietic stem cell, progeny thereof and/or cells that have differentiated from said HSC, e.g., lymphoid cells or myeloid cells. An immune cell can be an adaptive immune cell or an innate immune cell. Examples of immune cells include T cells, B cells, Natural Killer cells. Natural Killer T cells, neutrophils, dendritic cells, monocytes, macrophages, and granulocytes.

In some embodiments, an immune cell is a T cell. In some embodiments, a T cell includes a CD4+ T cell, a CD8+ T cell, a TCR alpha-beta T cell, a TCR gamma-delta T cell. In some embodiments, a T cell comprises a memory T cell (e.g., a central memory T cell, or an effector memory T cell (e.g., a TEMRA) or an effector T cell. In some embodiments, a T cell comprises a tumor infiltrating lymphocyte (TIL).

Described herein is a method for producing CM T cells comprising contacting a population of T cells with a molecule that binds to TCRβV, wherein the population of T cells are induced into CM T cells. Also described herein is a method of making a population of cells comprising: contacting a population of T cells with a molecule that binds to TCRβV: culturing the population of T cells in the presence of the molecule that binds to TCRβV for a time sufficient to produce the population of cells, wherein at least 50% of the population of cells are CM T cells. In some embodiments, the contacting is conducted ex vivo. In some embodiments, the contacting is conducted in vivo. In some embodiments, the method differentiates EM T cells to CM T cells. In some embodiments, the method differentiates T_{EMRA} T cells to CM T cells. In some embodiments, the molecule is a multispecific molecule. In some embodiments, the multispecific molecule comprises a cytokine molecule. In some embodiments, the molecule that binds to TCRβV is attached to a solid surface. In some embodiments, the solid surface is a bead or a plate. In some embodiments, culturing comprises culturing the population of T cells in the presence of IL-2 and/or X-VIVO culture media.

Described herein is a composition comprising CM T cells, which are produced by a method described in the present application.

The CM T cells described herein may be TCRβ3V+, CCR7+, CCR7−, CD45RA−, CD45RA+, CD95+, CD95−, CCR7+ and CD45RA−, CCR7− and CD45RA+, CD95+ and CCR7+, CD95− and CCR7, CD95+ and CD45RA−, CD95− and CD45RA+, CD95+, CCR7+ and CD45RA−, CD95−, CCR7− and CD45RA+, CD38+, CD38, CD25+, CD25−, CD38+ and CD25+, CD38− and CD25−, PD-1+, PD-1−, TIM-3+, TIM-3+, PD-1+ and TIM-3+, PD-1− and TIM-3−, IFNγ+, IFNγ−, TNFα+, TNFα−, IFNγ+ and TNFα+, IFNγ− and TNFα−, CD62L+, CD62L−, CD44+, CD44−, CD62L+ and CD44+, or CD62L− and CD44−.

In some embodiments, an immune cell is an NK cell.

In some embodiments, an immune cell is a TIL. TILs are immune cells (e.g., T cells. B cells or NK cells) that can be found in a tumor or around a tumor (e.g., in the stroma or tumor microenvironment of a tumor), e.g., a solid tumor, e.g., as described herein. TILs can be obtained from a sample from a subject having cancer, e.g., a biopsy or a surgical sample. In some embodiments. TILs can be expanded using a method as described herein. In some embodiments, a population of expanded TILs, e.g., expanded using a method as described herein, can be administered to a subject to treat a disease, e.g., a cancer.

In some embodiments, immune cells, e.g., T cells (e.g., TILs), can be obtained from a unit of blood collected from a subject using any number of techniques known to the skilled artisan, such as Ficoll™ separation. In one aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations. The methods described herein can include more than one selection step, e.g., more than one depletion step.

In one embodiment, the methods as described herein can utilize culture media conditions comprising DMEM, DMEM F12, RPMI 1640, and/or AIM V media. The media can be supplemented with glutamine, HEPES buffer (e.g., 10 mM), serum (e.g., heat-inactivated serum, e.g., 10%), and/or beta mercaptoethanol (e.g., 55 uM). In some embodiments, the culture conditions as described herein comprise one or more supplements, cytokines, growth factors, or hormones. In some embodiments, the culture condition comprises one or more of IL-2, IL-15, or IL-7, or a combination thereof.

Immune effector cells such as T cells may be activated and expanded generally using methods as described, for example, in U.S. Pat. Nos. 6,352,694; 6,534,055; or 6,905,680. Generally, a population of immune cells, may be expanded by contact with an agent that stimulates a CD3/TCR complex associated signal and a ligand that stimulates a costimulatory molecule on the surface of the T cells; and/or by contact with a cytokine, e.g., IL-2. IL-15 or IL-7. T cell expansion protocols can also include stimulation, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3. B-T3, XR-CD28 (Diaclone, Besangon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, a TIL population can also be expanded by methods known in the art. For example, a population of TILs can be expanded as described in Hall et al., Journal for ImmunoTherapy of Cancer (2016) 4:61, the entire contents of which are hereby incorporated by reference. Briefly. TILs can be isolated from a sample by mechanical and/or physical digestion. The resultant TIL population can be stimulated with an anti-CD3 antibody in the presence of non-dividing feeder cells. In some embodiments, the TIL population can be cultured, e.g., expanded, in the presence of IL-2, e.g., human IL-2. In some embodiments, the TIL cells can be cultured, e.g., expanded for a period of at least 1-21 days, e.g., at least 1, 2, 3, 4, 5, 6, 7, 8, 9, 10, 11, 12, 13, 14, 15, 16, 17, 18, 19, 20 or 21 days.

As described herein, in some embodiments, an immune cell population (e.g., a T cell (e.g., a TEMRA cell or a TIL population) can be expanded by contacting the immune cell population with the multifunctional polypeptide molecule as described herein.

In some embodiments, the expansion occurs in vivo, e.g., in a subject. In some embodiments, a subject is administered the multifunctional polypeptide molecule as described herein resulting in expansion of immune cells in vivo.

In some embodiments, the expansion occurs ex vivo, e.g., in vitro. In some embodiments, cells from a subject, e.g., T cells, e.g., TIL cells, are expanded in vitro with the multifunctional polypeptide molecule as described herein. In some embodiments, the expanded TILs are administered to the subject to treat a disease or a symptom of a disease.

In some embodiments, a method of expansion as described herein results in an expansion of at least 1.1-10 fold, 10-20 fold, or 20-50 fold expansion. In some embodiments, the expansion is at least 1.1, 1.2, 1.3, 1.4, 1.5, 2, 3, 4, 5, 6, 7, 8, 9, 10, 15, 20, 25, 30, 35, 40, 45, 50, 55, 60, 65, 70, 75, 80, 85, 90, 100, 200, 300, 400, 500, 600, 700, 800, 900, 1000, 1500, 2000, 2500, 3000, 3500, 400, 4500, 5000, 5500, 6000, 6500, 7000, 7500, 8000, 8500, 9000, 9500, or 10000 fold expansion.

In some embodiments, a method of expansion as described herein comprises culturing, e.g., expanding, the cells for at least about 4 hours, 6 hours, 10 hours, 12 hours, 15 hours, 18 hours, 20 hours, or 22 hours. In some embodiments, a method of expansion as described herein comprises culturing, e.g., expanding, the cells for at least 1, 2, 3, 4, 5, 6, 7, 8, 9, 10, 11, 12, 13, 14, 15, 16, 17, 18, 19, 20 or 21 days. In some embodiments, a method of expansion as described herein comprises culturing, e.g., expanding, the cells for at least about 1 week, 2 weeks, 3 weeks, 4 weeks, 5 weeks, 6 weeks, 7 weeks or 8 weeks.

In some embodiments, a method of expansion as described herein is performed on immune cells obtained from a healthy subject.

In some embodiments, a method of expansion as described herein is performed on immune cells (e.g., TILs) obtained from a subject having a disease, e.g., a cancer, e.g., a solid tumor as described herein.

In some embodiments, a method of expansion as described herein further comprises contacting the population of cells with an agent, that promotes, e.g., increases, immune cell expansion. In some embodiments, the agent comprises an immune checkpoint inhibitor, e.g., a PD-1 inhibitor, a LAG-3 inhibitor, a CTLA4 inhibitor, or a TIM-3 inhibitor. In some embodiments, the agent comprises a 4-1BB agonist, e.g., an anti-4-1BB antibody.

Without wishing to be bound by theory, in some embodiments, the multifunctional polypeptide molecule as described herein can expand, e.g., selectively or preferentially expand. T cells expressing a T cell receptor (TCR) comprising a TCR alpha and/or TCR beta molecule, e.g., TCR alpha-beta T cells (αβ T cells). In some embodiments, the multifunctional polypeptide molecule as described herein does not expand, or induce proliferation of T cells expressing a TCR comprising a TCR gamma and/or TCR delta molecule, e.g., TCR gamma-delta T cells (γδ T cells). In some embodiments, the multifunctional polypeptide molecule as described herein selectively or preferentially expands as T cells over γδ T cells.

Without wishing to be bound by theory, in some embodiments, γδ T cells are associated with cytokine release syndrome (CRS) and/or neurotoxicity (NT). In some embodiments, the multispecific or multifunctional molecules as described herein result in selective expansion of non-γδ T cells, e.g., expansion of αβ T cells, thus reducing CRS and/or NT.

In some embodiments, any of the compositions or methods as described herein result in an immune cell population having a reduction of, e.g., depletion of, 7γδ T cells. In some embodiments, the immune cell population is contacted with an agent that reduces, e.g., inhibits or depletes, γδ T cells, e.g., an anti-IL-17 antibody or an agent that binds to a TCR gamma and/or TCR delta molecule.

In some embodiments, the multifunctional polypeptide molecule as described herein results in expansion of an immune cell, e.g., a T cell, a tumor infiltrating lymphocyte (TIL), an NK cell, or other immune cells (e.g., as described herein).

In some embodiments, binding of the multifunctional polypeptide molecule as described herein to a TCRβV region results in one, two, three or all of: (i) reduced T cell proliferation kinetics; (ii) cell killing, e.g., target cell killing, e.g. cancer cell killing, e.g., as measured by an assay of Example 4; (iii) increased Natural Killer (NK) cell proliferation, e.g., expansion; or (iv) expansion, e.g., at least about 1.1-10 expansion (e.g., at least about 1.1, 1.2, 1.3, 1.4, 1.5, 2, 3, 4, 5, 6, 7, 8, 9, or 10 fold expansion), of a population of T cells having a memory-like phenotype, e.g., as described herein, e.g., wherein (i)-(iv) are relative to the non-TCRβV-binding T cell engager.

In some embodiments, the method further comprises contacting the population of cells with an agent that promotes, e.g., increases, immune cell expansion. In some embodiments, the agent includes an immune checkpoint inhibitor, e.g., as described herein. In some embodiments, the agent includes a 4-1BB (CD127) agonist, e.g., an anti-4-1BB antibody.

In some embodiments, the method further comprises comprising contacting the population of cells with a non-dividing population of cells, e.g., feeder cells, e.g., irradiated allogenic human PBMCs.

In some embodiments, expansion of the population of immune cells, is compared to expansion of a similar population of cells with an antibody that binds to: a CD3 molecule, e.g., CD3 epsilon (CD3e) molecule; or a TCR alpha (TCRα) molecule.

In some embodiments, expansion of the population of immune cells, is compared to expansion of a similar population of cells not contacted with the anti-TCRβV antibody molecule or the multispecific or multifunctional molecules as described herein.

In some embodiments, expansion of the population of memory effector T cells, e.g., TEM cells. e.g., TEMRA cells, is compared to expansion of a similar population of cells with an antibody that binds to: a CD3 molecule, e.g., CD3 epsilon (CD3e) molecule; or a TCR alpha (TCRα) molecule.

In some embodiments, the method results in expansion of, e.g., selective or preferential expansion of, T cells expressing a T cell receptor (TCR) comprising a TCR alpha and/or TCR beta molecule, e.g., TCR alpha-beta T cells (up T cells).

In some embodiments, the method results in expansion of αβT cells over expansion of T cells expressing a TCR comprising a TCR gamma and/or TCR delta molecule, e.g., TCR gamma-delta T cells (γδ T cells). In some embodiments, expansion of αβT cells over γδ T cells results in reduced production of cytokines associated with CRS. In some embodiments, expansion of αβT cells over γδ T cells results in immune cells that have reduced capacity to, e.g., are less prone to, induce CRS upon administration into a subject.

In some embodiments, an immune cell population (e.g., T cells (e.g., TEMRA cells or TILs) or NK cells) cultured in the presence of, e.g., expanded with, the multifunctional polypeptide molecule as described herein does not induce CRS and/or NT when administered into a subject, e.g., a subject having a disease or condition as described herein.

In some embodiments, provided herein is a multifunctional polypeptide molecule as described herein comprising a non-murine, e.g., a human-like antibody molecule (e.g., a human or humanized antibody molecule), which binds, e.g., specifically binds, to a T cell receptor beta variable (TCRβV) region. In some embodiments, binding of the multifunctional polypeptide molecule as described herein results in expansion, e.g., at least about 1.1-50 fold expansion (e.g., at least about 1.540 fold, 2-35 fold, 3-30 fold, 5-25 fold, 8-20 fold, or 10-15 fold expansion), of a population of T cells, e.g., a population of T cells having a memory-like phenotype, e.g., CD45RA+CCR7− T cells. In some embodiments, the population of T cells having a memory-like phenotype comprises CD4+ and/or CD8+ T cells. In some embodiments, the population of T cells having a memory-like phenotype comprises a population of memory T cells, e.g., T effector memory (TEM) cells, e.g., TEM cells expressing CD45RA (TEMRA) cells, e.g., CD4+ or CD8+ TEMRA cells. In some embodiments, the population of T cells having a memory-like phenotype does not express a senescent marker, e.g., CD57. In some embodiments, the population of T cells having a memory-like phenotype does not express an inhibitory receptor, e.g., OX40, 4-1BB, and/or ICOS.

In some embodiments, the population of T cells having a memory-like phenotype is a population of T cells with CD45RA+CCR7− CD57−. In some embodiments, the population of T cells having a memory-like phenotype does not express an inhibitory receptor, e.g., OX40, 4-1BB, and/or ICOS.

In some embodiments, the population of T cells having a memory-like phenotype, e.g., as described herein, has increased proliferative capacity, e.g., as compared to a reference cell population, e.g., an otherwise similar population of cells that has not been contacted with an anti-TCRβV antibody or the multispecific or multifunctional molecules as described herein.

In some embodiments, the expansion is at least about 1.1-10 fold expansion (e.g., at least about 1.1, 1.2, 1.3, 1.4, 1.5, 2, 3, 4, 5, 6, 7, 8, 9, or 10 fold expansion).

In some embodiments, expansion of the population of T cells having a memory-like phenotype. e.g., memory effector T cells, e.g., TEM cells, e.g., TEMRA cells, e.g., CD4+ or CD8+ TEMRA cells, is compared to expansion of a similar population of cells with an antibody that binds to: a CD3 molecule. e.g., CD3 epsilon (CD3e) molecule; or a TCR alpha (TCRa) molecule.

In some embodiments, the population of expanded T cells having a memory-like phenotype, e.g., T effector memory cells, comprises cells T cells, e.g., CD3+, CD8+ or CD4+ T cells. In some embodiments, the population of expanded T cells having a memory-like phenotype. T effector memory cells, comprises CD3+ and CD8+ T cells. In some embodiments, the population of expanded T cells having a memory-like phenotype, e.g., T effector memory cells comprises CD3+ and CD4+ T cells.

In some embodiments, the population of expanded T cells having a memory-like phenotype, T effector memory (TEM) cells, comprises cells T cells, e.g., CD3+, CD8+ or CD4+ T cells, which express or re-express, CD45RA, e.g., CD45RA+. In some embodiments, the population comprises TEM cells expressing CD45RA, e.g., TEMRA cells. In some embodiments, expression of CD45RA on TEMRA cells, e.g., CD4+ or CD8+ TEMRA cells, can be detected by a method as described herein, e.g., flow cytometry.

In some embodiments, the population of T cells having a memory-like phenotype, e.g., TEMRA cells have low or no expression of CCR7, e.g., CCR7− or CCR7 low. In some embodiments, expression of CCR7 on TEMRA cells cannot be detected by a method as described herein, e.g., flow cytometry.

In some embodiments, the population of T cells having a memory-like phenotype, e.g., TEMRA cells express CD95, e.g., CD95+. In some embodiments, expression of CD95 on TEMRA cells can be detected by a method as described herein, e.g., flow cytometry.

In some embodiments, the population of T cells having a memory-like phenotype, e.g., TEMRA cells express CD45RA, e.g., CD45RA+, have low or no expression of CCR7, e.g., CCR7− or CCR7 low, and express CD95, e.g., CD95+. In some embodiments, the population of T cells having a memory-like phenotype, e.g., TEMRA cells can be identified as CD45RA+, CCR7− and CD95+cells. In some embodiments, the population of T cells having a memory-like phenotype, e.g., TEMRA cells comprise CD3+, CD4+ or CD8+ T cells (e.g., CD3+ T cells, CD3+CD8+ T cells, or CD3+CD4+ T cells).

In some embodiments, the population of T cells having a memory-like phenotype does not express a senescent marker, e.g., CD57.

In some embodiments, the population of T cells having a memory-like phenotype does not express an inhibitory receptor, e.g., OX40, 4-1BB, and/or ICOS.

In some embodiments, binding of the multifunctional polypeptide molecule as described herein results in expansion, e.g., at least about 1.1-50 fold expansion (e.g., at least about 1.540 fold, 2-35 fold, 3-30 fold, 5-25 fold, 8-20 fold, or 10-15 fold expansion), of a subpopulation of T cells. In some embodiments, the multifunctional polypeptide molecule as described herein-activated (e.g., expanded) subpopulation of T cells resemble TEMRA cells in high expression of CD45RA and/or low expression of CCR7. In some embodiments, the multifunctional polypeptide molecule as described herein-activated (e.g., expanded) subpopulation of T cells do not display upregulation of the senescence markers CD57 and/or KLRG1. In some embodiments, the multifunctional polypeptide molecule as described herein-activated (e.g., expanded) subpopulation of T cells do not display upregulation of co-stimulatory molecules CD27 and/or CD28. In some embodiments, the multifunctional polypeptide molecule as described herein-activated (e.g., expanded) subpopulation of T cells are highly proliferative. In some embodiments, the multifunctional polypeptide molecule as described herein-activated (e.g., expanded) subpopulation of T cells secrete IL-2. In some embodiments, expression of surface markers on T cells can be detected by a method as described herein, e.g., flow cytometry. In some embodiments, the proliferative capability of T cells can be detected by a method as described herein, e.g., a method described in Example 4. In some embodiments, cytokine expression of T cells can be detected by a method as described herein, e.g., a method described in Examples 10 and 35. In some embodiments, the expansion is at least about 1.1-10 fold expansion (e.g., at least about 1.1, 1.2, 1.3, 1.4, 1.5, 2.3, 4, 5, 6, 7.8.9, or 10 fold expansion). In some embodiments, the expansion is compared to expansion of a similar population of cells with an antibody that binds to a CD3 molecule, e.g., CD3 epsilon (CD3e) molecule: or a TCR alpha (TCRa) molecule.

In some embodiments, binding of the multifunctional polypeptide molecule as described herein results in proliferation, e.g., expansion, e.g., at least about 1.1-50 fold expansion (e.g., at least about 1.5-40 fold, 2-35 fold, 3-30 fold, 5-25 fold, 8-20 fold, or 10-15 fold expansion), of a population of Natural Killer (NK) cells. In some embodiments, the expansion of NK cells is at least about 1.1-30 fold expansion (e.g., at least about 1.1, 1.2, 1.3, 1.4, 1.5, 2, 3, 4, 5, 6, 7, 8, 9, 10, 15, 20, 25, or at least about 1.1-5, 5-10, 10-15, 15-20, 20-25, or 25-30 fold expansion). In some embodiments, the expansion of NK cells is measure by an assay of Example 4. In some embodiments, the expansion of NK cells by, e.g., binding of, the multifunctional polypeptide molecule as described herein is compared to expansion of an otherwise similar population not contacted with the multifunctional polypeptide molecule as described herein.

In some embodiments, binding of the multifunctional polypeptide molecule as described herein results in cell killing, e.g., target cell killing, e.g. cancer cell killing. In some embodiments, the cancer cell is a hematological cancer cell or a solid tumor cell. In some embodiments, the cancer cell is a multiple myeloma cell. In some embodiments, binding of the multifunctional polypeptide molecule as described herein results in cell killing in vitro or in vivo. In some embodiments, cell killing is measured by an assay of Example 4.

In some embodiments, binding of the multifunctional polypeptide molecule as described herein to a TCRβV region results in an increase or decrease of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) of any of the activities described herein compared the activity of 16G8 or TM23 murine antibody, or a humanized version thereof as described in U.S. Pat. No. 5,861,155.

In some embodiments, the method comprises expanding, e.g., increasing the number of, an immune cell population in the subject. In some embodiments, provided herein is a method of expanding, e.g., increasing the number of, an immune cell population comprising, contacting the immune cell population with an effective amount of the multifunctional polypeptide molecule as described herein. In some embodiments, the expansion occurs in vivo or ex vivo (e.g., in vitro).

In some embodiments, provided herein is a method of expanding, e.g., increasing the number of, an immune cell population comprising, contacting the immune cell population with a multifunctional polypeptide molecule as described herein comprising an antibody molecule, e.g., humanized antibody molecule, which binds, e.g., specifically binds, to a T cell receptor beta variable chain (TCRβV) region (e.g., anti-TCRβV antibody molecule), thereby expanding the immune cell population. In some embodiments, the expansion occurs in vivo or ex vivo (e.g., in vitro).

In some embodiments, provided herein is a method of expanding a population of immune effector cells from a subject having a cancer, the method comprising: (i) isolating a biological sample comprising a population of immune effector cells from the subject; e.g., a peripheral blood sample, biopsy sample, or bone marrow sample; (ii) acquiring a value of the status of one or more TCRβV molecules for the subject, e.g., in the biological sample from the subject, wherein said value comprises a measure of the presence of, e.g., level or activity of, a TCRβV molecule in a sample from the subject compared to a reference value, e.g., a sample from a health subject, wherein a value that is higher, e.g., increased, in the subject relative to the reference, e.g., healthy subject, is indicative of the presence of cancer in the subject, and (iii) contacting the biological sample comprising a population of immune effector cells with the multifunctional poly peptide molecule as described herein.

In some embodiments, the method further comprises administering the population of immune effector cells contacted with the multifunctional polypeptide molecule as described herein to the subject.

In some embodiments, a higher, e.g., increased, level or activity of one or more TCRβV molecules in a subject, e.g., in a sample from a subject, is indicative of a bias, e.g., a preferential expansion, e.g., clonal expansion, of T cells expressing said one or more TCRβV molecules in the subject.

In some embodiments, the anti-TCRvβ bifunctional or bispecific antibody molecule induces TCR signalling and IL-2R pathway activation in human T cells that precedes expansion of Vβ6/Vβ10 T cell subsets to 80-90% of the T cell compartment. In some embodiments, the anti-TCRvβ bifunctional or bispecific antibody molecule induces TCR signalling and IL-2R pathway activation in human T cells that precedes expansion of Vβ6/β10 T cell subsets to at least 5%, 10%, 15%, 20%, 25%, 30%, 35%, 40%, 45%, 50%, 55%, 60%, 65%, 70%, 75%, 80%, 85%, 90%, 95%, 99%, or 99.5% of the T cell compartment. In some embodiments, the anti-TCRvβ bifunctional or bispecific antibody molecule induces TCR signalling and IL-2R pathway activation in human T cells that precedes expansion of Vβ6/Vβ10 T cell subsets to at most 5%, 10%, 15%, 20%, 25%, 30%, 35%, 40%, 45%, 50%, 55%, 60%, 65%, 70%, 75%, 80%, 85%, 90%, 95%, 99%, or 99.5% of the T cell compartment. In some embodiments, the anti-TCRvβ bifunctional or bispecific antibody molecule induces TCR signalling and IL-2R pathway activation in human T cells that precedes expansion of Vβ6/Vβ10 T cell subsets to 5-15%, 5-20%, 5-25%, 5-30%, 5-35%, 5-40%, 5-45%, 5-50%, 5-55%, 5-65%, 5-70%, 5-75%, 5-80%, 5-85%, 5-90%, 5-95%, 5-99%, or 5-99.5% of the T cell compartment. In some embodiments, the anti-TCRvβ bifunctional or bispecific antibody molecule induces TCR signalling and IL-2R pathway activation in human T cells that precedes expansion of Vβ6/Vβ10 T cell subsets to 10-99.5%, 15-99.5%, 20-99.5%, 25-99.5%, 30-99.5%, 35-99.5%, 40-99.5%, 45-99.5%, 50-99.5%, 55-99.5%, 60-99.5%, 65-99.5%, 70-99.5%, 75-99.5%, 80-99.5%, 85-99.5%, 90-99.5%, or 95-99.5% of the T cell compartment. In some embodiments, the anti-TCRvβ bifunctional or bispecific antibody molecule induces TCR signalling and IL-2R pathway activation in human T cells that precedes expansion of Vβ6/Vβ10 T cell subsets to 5-15%, 10-20/o, 15-25%, 20-30%, 25-35%, 30-40%, 35-45%, 40-50%, 45-55%, 50-60%, 55-65%, 60-70%, 65-75%, 70-80%, 75-85%, 80-90%, 85-95%, or 90-99.5% of the T cell compartment.

In some embodiments, the anti-TCRvβ bifunctional or bispecific antibody molecule induces expansion of Vβ6/Vβ10 T cell subsets to 80-90% of the T cell compartment. In some embodiments, the anti-TCRvβ bifunctional or bispecific antibody molecule induces expansion of Vβ6/Vβ10 T cell subsets to at least 5%, 10%, 15%, 20%, 25%, 30%, 35%, 40%, 45%, 50%, 55%, 60%, 65%, 70%, 75%, 80%, 85%, 90%, 95%, 99%, or 99.5% of the T cell compartment. In some embodiments, the anti-TCRvβ bifunctional or bispecific antibody molecule induces expansion of Vβ6/Vβ10 T cell subsets to at most 5%, 10%, 15%, 20%, 25%, 30%, 35%, 40%, 45%, 50%, 55%, 60%, 65%, 70%, 75%, 80%, 85%, 90%, 95%, 99%, or 99.5% of the T cell compartment. In some embodiments, the anti-TCRvβ bifunctional or bispecific antibody molecule induces expansion of Vβ6/Vβ10 T cell subsets to 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 5-99%, or 5-99.5% of the T cell compartment. In some embodiments, the anti-TCRvβ bifunctional or bispecific antibody molecule induces expansion of Vβ6/Vβ10 T cell subsets to 10-99.5%, 15-99.5%, 20-99.5%, 25-99.5%, 30-99.5%, 35-99.5%, 40-99.5%, 45-99.5%, 50-99.5%, 55-99.5%, 60-99.5%, 65-99.5%, 70-99.5%, 75-99.5%, 80-99.5%, 85-99.5%, 90-99.5%, or 95-99.5% of the T cell compartment. In some embodiments, the anti-TCRvD bifunctional or bispecific antibody molecule induces expansion of Vβ6/Vβ10 T cell subsets to 5-15%, 10-20%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95%, or 90-99.5% of the T cell compartment.

In some embodiments, 80-90% of the T cells stimulated with the anti-TCRvβ bifunctional or bispecific antibody molecule adopts a novel, activated central memory (TCM)-like phenotype. In some embodiments, at least 5%, 10%, 15%, 20%, 25%, 30%, 35%, 40%, 45%, 50%, 55%, 60%, 65%, 70%, 75%, 80%, 85%, 90%, 95%, 99%, or 99.5% of the T cells stimulated with the anti-TCRvβ bifunctional or bispecific antibody molecule adopts a novel, activated central memory (TCM)-like phenotype. In some embodiments, at most 5%, 10%, 15%, 20/o, 25%, 30%, 35%, 40%, 45%, 50%, 55%, 60%, 65%, 70%, 75%, 80%, 85%, 90%, 95%, 99%, or 99.5% of the T cells stimulated with the anti-TCRvβ bifunctional or bispecific antibody molecule adopts a novel, activated central memory (TCM)-like phenotype. In some embodiments. 5-15%, 5-20%, 5-25%, 5-30%, 5-35%, 5-40%, 545%, 5-50%, 5-55%, 5-60%, 5-65%, 5-70%, 5-75%, 5-80%, 5-85%, 5-90%, 5-95%, 5-99%, or 5-99.5% of the T cells stimulated with the anti-TCRvβ bifunctional or bispecific antibody molecule adopts a novel, activated central memory (TCM)-like phenotype. In some embodiments, 10-99.5%, 15-99.5%, 20-99.5%, 25-99.5%, 30-99.5%, 35-99.5%, 40-99.5%, 45-99.5%, 50-99.5%, 55-99.5%, 60-99.5%, 65-99.5%, 70-99.5%, 75-99.5%, 80-99.5%, 85-99.5%, 90-99.5%, or 95-99.5% of the T cells stimulated with the anti-TCRvβ bifunctional or bispecific antibody molecule adopts a novel, activated central memory (TCM)-like phenotype. In some embodiments. 5-15%, 10-20%, 15-25%, 20-30%, 25-35%, 30-40%, 3545%, 40-50%, 45-55%, 50-60%, 55-65%, 60-70%, 65-75%, 70-80%, 75-85%, 80-90%, 85-95%, or 90-99.5% of the T cells stimulated with the anti-TCRvβ bifunctional or bispecific antibody molecule adopts a novel, activated central memory (TCM)-like phenotype.

In some embodiments, the anti-TCRvβ bifunctional or bispecific antibody molecule induces 60-70% tumor growth inhibition. In some embodiments, the anti-TCRvβ bifunctional or bispecific antibody molecule induces at least 5%, 10%, 15%, 20%, 25%, 30%, 35%, 40%, 45%, 50%, 55%, 60%, 65%, 70%, 75%, 80%, 85%, 90%, 95%, 99%, 99.5%, or 100% tumor growth inhibition. In some embodiments, the anti-TCRvβ bifunctional or bispecific antibody molecule induces at most 5%, 10%, 15%, 20%, 25%, 30%, 35%, 40%, 45%, 50%, 55%, 60%, 65%, 70%, 75%, 80%, 85%, 90%, 95%, 99%, 99.5%, or 100% tumor growth inhibition. In some embodiments, the anti-TCRvβ bifunctional or bispecific antibody molecule induces 5-15%, 5-20%, 5-25%, 5-30%, 5-35%, 5-40%, 545%, 5-50%, 5-55%, 5-60%, 5-65%, 5-70%, 5-75%, 5-80%, 5-85%, 5-90%, 5-95%, 5-99%, 5-99.5%, or 5-100% tumor growth inhibition. In some embodiments, the anti-TCRvβ bifunctional or bispecific antibody molecule induces 10-99.5%, 15-99.5%, 20-99.5%, 25-99.5%, 30-99.5%, 35-99.5%, 40-99.5%, 45-99.5%, 50-99.5%, 55-99.5%, 60-99.5%, 65-99.5%, 70-99.5%, 75-99.5%, 80-99.5%, 85-99.5%, 90-99.5%, or 95-99.5% tumor growth inhibition. In some embodiments, the anti-TCRvβ bifunctional or bispecific antibody molecule induces 10-100%, 15-100%, 20-100%, 25-100%, 30-100/o, 35-100%, 40-100%, 45-100%, 50-100%, 55-100%, 60-100%, 65-100%, 70-100%, 75-100%, 80-100%, 85-100%, 90-100%, or 95-100% tumor growth inhibition. In some embodiments, the anti-TCRvβ bifunctional or bispecific antibody molecule induces 5-15%, 10-20%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95%, 90-99.5%, or 90-100% tumor growth inhibition.

In some embodiments, the anti-TCRvβ bifunctional or bispecific antibody molecule induces 60-70% tumor growth inhibition with long-term protection from tumor rechallenge. In some embodiments, the anti-TCRvβ bifunctional or bispecific antibody molecule induces at least 5%, 10%, 15%, 20%, 25%, 30%, 35%, 40%, 45%, 50%, 55%, 60%, 65%, 70%, 75%, 80%, 85%, 90/o, 95%, 99%, 99.5%, or 100% tumor growth inhibition with long-term protection from tumor rechallenge. In some embodiments, the anti-TCRvβ bifunctional or bispecific antibody molecule induces at most 5%, 10%, 15%, 20%, 25%, 30%, 35%, 40%, 45%, 50%, 55%, 60%, 65%, 70%, 75%, 80%, 85%, 90%, 95%, 99%, 99.5%, or 100% tumor growth inhibition with long-term protection from tumor rechallenge. In some embodiments, the anti-TCRvβ bifunctional or bispecific antibody molecule induces 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 5-99%, 5-99.5%, or 5-100% tumor growth inhibition with long-term protection from tumor rechallenge. In some embodiments, the anti-TCRvβ bifunctional or bispecific antibody molecule induces 10-99.5%, 15-99.5%, 20-99.5%, 25-99.5%, 30-99.5%, 35-99.5%, 40-99.5%, 45-99.5%, 50-99.5%, 55-99.5%, 60-99.5%, 65-99.5%, 70-99.5%, 75-99.5%, 80-99.5%, 85-99.5%, 90-99.5%, or 95-99.5% tumor growth inhibition with long-term protection from tumor rechallenge. In some embodiments, the anti-TCRvβ bifunctional or bispecific antibody molecule induces 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, or 95-100% tumor growth inhibition with long-term protection from tumor rechallenge. In some embodiments, the anti-TCRvβ bifunctional or bispecific antibody molecule induces 5-15%, 10-20%, 15-25%, 20-30%, 25-35%, 30-40%, 3545%, 40-50%, 45-55%, 50-60%, 55-65%, 60-7(0%, 65-75%, 70-80%, 75-85%, 80-90%, 85-95%, 90-99.5%, or 90-100% tumor growth inhibition with long-term protection from tumor rechallenge.

In some embodiments, the Vβ T cells targeted by the anti-TCRvβ bifunctional or bispecific antibody molecule is present as 10-12% of TILs. In some embodiments, the Vβ T cells targeted by the anti-TCRvβ bifunctional or bispecific antibody molecule is present as at least 1%, 2%, 3%, 4%, 5%, 6%, 7%, 8%, 9%, 10%, 11%, 12%, 13%, 14%, 15%, 16%, 17%, 18%, 19%, 20%, 22%, 24%, 26%, 28%, 30%, 32%, 34%, 36%, 38%, 40%, 42%, 44%, 46%, 48%, 50%, 55%, 60%, 65%, 70%, 75%, 80%, 85%, 90%, or 95% of TILs. In some embodiments, the Vβ T cells targeted by the anti-TCRvβ bifunctional or bispecific antibody molecule is present as at most 1%, 2%, 3%, 4%, 5%, 6%, 7%, 8%, 9%, 10%, 11%, 12%, 13%, 14%, 15%, 16%, 17%, 18%, 19%, 20%, 22%, 24%, 26%, 28%, 30%, 32%, 34%, 36%, 38%, 40%, 42%, 44%, 46%, 48%, 50%, 55%, 60%, 65%, 70%, 75%, 80%, 85%, 90%, or 95% of TILs. In some embodiments, the Vβ T cells targeted by the anti-TCRvβ bifunctional or bispecific antibody molecule is present as 1-2%, 1-3%, 1-4%, 1-5%, 1-6%, 1-7%, 1-8%, 1-9%, 1-10%, 1-11%, 1-12%, 1-13%, 1-14%, 1-15%, 1-16%, 1-17%, 1-18%, 1-19%, 1-20%, 1-22%, 1-24%, 1-26%, 1-28%, 1-30%, 1-32%, 1-34%, 1-36%, 1-38%, 1-40%, 1-42%, 1-44%, 1-46%, 1-48%, 1-50%, 1-55%, 1-60%, 1-65%, 1-70%, 1-75%, 1-80%, 1-85%, 1-90%, or 1-95% of TILs. In some embodiments, the Vβ T cells targeted by the anti-TCRvβ bifunctional or bispecific antibody molecule is present as 1-95%, 2-95%, 3-95%, 4-95%, 5-95%, 6-95%, 7-95%, 8-95%, 9-95%, 10-95%, 11-95%, 12-95%, 13-95%, 14-95%, 15-95%, 16-95%, 17-95%, 18-95%, 19-95%, 20-95%, 22-95%, 24-95%, 26-95%, 28-95%, 30-95%, 32-95%, 34-95%, 36-95%, 38-95%, 40- 95%, 42-95%, 44-95%, 46-95%, 48-95%, 50-95%, 55-95%, 60-95%, 65-95%, 70-95%, 75-95%, 80-95%, 85-95%, or 90-95% of TILs. In some embodiments, the Vβ T cells targeted by the anti-TCRvβ bifunctional or bispecific antibody molecule is present as 1-3%, 2-4%, 3-5%, 4-6%, 5-7%, 6-8%, 7-9%, 8-10%, 9-11%, 10-12%, 11-13%, 12-14%, 13-15%, 14-16%, 15-17%, 16-18%, 17-19%, 18-20%, 19-21%, 20-22%, 22-24%, 24-26%, 26-28%, 28-30%, 30-32%, 32-34%, 34-36%, 36-38%, 38-40%, 40-42%, 42- 44%, 44-46%, 46-48%, 48-50%, 50-55%, 55-60%, 60-65%, 65-70%, 70-75%, 75-80%, 80-85%, 85-90%, or 90-95% of TILs.

In some embodiments, stimulation of T cells with the anti-TCRvβ bifunctional or bispecific antibody molecule results in potent expansion with approximately 80% adopting a novel memory phenotype. In some embodiments, stimulation of T cells with the anti-TCRvβ bifunctional or bispecific antibody molecule results in potent expansion with approximately at least 5%, 10%, 15%, 20%, 25%, 30%, 35%, 40%, 45%, 50%, 55%, 60%, 65%, 70%, 75%, 80%, 85%, 90%, 95%, 99%, 99.5%, or 100% adopting a novel memory phenotype. In some embodiments, stimulation of T cells with the anti-TCRvβ bifunctional or bispecific antibody molecule results in potent expansion with approximately at most 5%, 10%, 15%, 20%, 25%, 30%, 35%, 40%, 45%, 50%, 55%, 60%, 65%, 70%, 75%, 80%, 85%, 90%, 95%, 99%, 99.5%, or 100% adopting a novel memory phenotype. In some embodiments, stimulation of T cells with the anti-TCRvβ bifunctional or bispecific antibody molecule results in potent expansion with approximately 5%, 10%, 15%, 20%, 25%, 30%, 35%, 40%, 45%, 50%, 55%, 60%, 65%, 70%, 75%, 80%, 85%, 90%, 95%, 99%, 99.5%, or 100% adopting a novel memory phenotype.

In some embodiments, the binding of T cells to a molecule that binds to a TCRβV (e.g., an anti-TCRvβ antibody) induces expansion of Vβ T cell subsets (e.g., Vβ6/Vβ10 T cell subsets) preferentially in CD8⁺ T cells over CD4⁺ T cells. In some embodiments, a molecule that binds to a TCRβV (e.g., an anti-TCRvβ antibody) induces an activated phenotype with markers of both effector and central memory T cells (T_{EM}/T_{CM}), compared with controls and anti-CD3 mAbs. In some embodiments, a molecule that binds to a TCRβV (e.g., an anti-TCRvβ antibody) boosts the ex vivo expansion of antigen-specific T cells obtained from individuals (e.g., HPV⁺ individuals). In some embodiments, a molecule that binds to a TCRβV (e.g., an anti-TCRvβ antibody) eradicates tumors, leads to substantial tumor regressions, or a combination thereof. In some embodiments, a subject treated using a molecule that binds to a TCRβV (e.g., an anti-TCRvβ antibody), a population of immune cells in which at least 50% of the cells are central memory (CM) T cells induced by binding to a molecule that binds to a TCRβV, or a pharmaceutical composition comprising such a population of immune cells shows long-term protection from tumor re-challenge. In some embodiments, the anti-tumor activity using a molecule that binds to a TCRβV (e.g., an anti-TCRvβ antibody), a population of immune cells in which at least 50% of the cells are central memory (CM) T cells induced by binding to a molecule that binds to a TCRβV, or a pharmaceutical composition comprising such population of immune cells is dependent on the accumulation of Vβ T cell subsets (e.g., Vβ6/Vβ10 T cells). In some embodiments, expanded Vβ T cell subsets (e.g., Vβ6/Vβ10 T cells) are almost exclusively polyclonal T_{EM} or T_{CM} cells, with almost no exhausted or regulatory T cells. In some embodiments, expanded Vβ T cell subsets (e.g., Vβ6/Vβ10 T cells) are exclusively polyclonal T_{EM} or T_{CM} cells, with almost no exhausted or regulatory T cells. In some embodiments, expanded Vβ T cell subsets (e.g., Vβ6/Vβ10 T cells) are almost exclusively poly clonal T_{EM} or T_{CM} cells. In some embodiments, expanded Vβ T cell subsets (e.g., Vβ6/Vβ10 T cells) are exclusively polyclonal T_{EM} or T_{CM} cells. In some embodiments, expanded Vβ T cell subsets (e.g., Vβ6/Vβ10 T cells) show almost no exhausted or regulatory T cells. In some embodiments, expanded Vβ T cell subsets (e.g., Vβ6/Vβ10 T cells) show no exhausted or regulatory T cells.

In some embodiments, an exemplary T cell-activating antibody molecule that selectively targets the TCRβ chain promotes antitumor activity through activation and expansion of a polyclonal effector memory T cell subset. In some embodiments, a multispecific molecule containing a molecule that binds to a TCRβV (e.g., an anti-TCRvβ antibody) and an cytokine (e.g., an IL-2 domain) selectively activates and expands a subset of human αβ T cells expressing the germline-encoded specific TCRs that are enriched in tumor infiltrating lymphocytes. In some embodiments, a multispecific molecule containing a molecule that binds to a TCRβV (e.g., an anti-TCRvβ antibody) and an cytokine (e.g., an IL-2 domain) simultaneously engages a non-clonal mode of TCR activation with cytokine co-stimulation. In some embodiments, In some embodiments, a multispecific molecule containing a molecule that binds to a TCRβV (e.g., an anti-TCRvβ antibody) and an cytokine (e.g., an IL-2 domain) induces TCR signalling and the cytokine pathway activation (e.g., IL-2R pathway activation) in human T cells that preceded expansion of Vβ T cell subsets (e.g., Vβ6/Vβ10 T cell subsets). In some embodiments, human T cells stimulated by a multispecific molecule containing a molecule that binds to a TCRβV (e.g., an anti-TCRvβ antibody) and an cytokine (e.g., an IL-2 domain) adopts an activated central memory (TCM)-like phenotype. In some embodiments, a multispecific molecule containing a molecule that binds to a TCRβV (e.g., an anti-TCRvβ antibody) and an cytokine (e.g., an IL-2 domain) eradicates tumors, led to substantial regressions of tumor growth with long-term protection from tumor rechallenge, or a combination thereof. In some embodiments, the anti-tumor activity of a multispecific molecule containing a molecule that binds to a TCRβV (e.g., an anti-TCRvβ antibody) and an cytokine (e.g., an IL-2 domain) is dependent on the accumulation of Vβ T cell subsets. In some embodiments, expanded Vβ T cells using a multispecific molecule containing a molecule that binds to a TCRβV (e.g., an anti-TCRvβ antibody) and an cytokine (e.g., an IL-2 domain) are almost exclusively polyclonal effector memory T cells (TEM) or TCM cells with minimal exhausted T cells or Tregs. In some embodiments, expanded Vβ T cells using a multispecific molecule containing a molecule that binds to a TCRβV (e.g., an anti-TCRvβ antibody) and an cytokine (e.g., an IL-2 domain) are exclusively polyclonal effector memory T cells (TEM) or TCM cells with minimal exhausted T cells or Tregs. In some embodiments, expanded Vβ T cells using a multispecific molecule containing a molecule that binds to a TCRβV (e.g., an anti-TCRvβ antibody) and an cytokine (e.g., an IL-2 domain) are associated with a novel gene signature comprising upregulation of memory and effector programs, and downregulation of exhaustion pathways.

In some embodiments, a multispecific molecule containing a molecule that binds to a TCRβV (e.g., an anti-TCRvβ antibody) and an cytokine (e.g., an IL-2 domain) selectively binds and activates subsets of the germline TCR repertoire. In some embodiments, a multispecific molecule containing a molecule that binds to a TCRβV (e.g., an anti-TCRvβ antibody) and an cytokine (e.g., an IL-2 domain) promotes a novel T cell phenotype with hallmarks of both effector and central memory cells. In some embodiments, a multispecific molecule containing a molecule that binds to a TCRβV (e.g., an anti-TCRvy antibody) and an cytokine (e.g., an IL-2 domain) provides potent and durable single-agent anti-tumor activity in solid tumor models that is dependent on expanded Vβ T cells. In some embodiments, a multispecific molecule containing a molecule that binds to a TCRβV (e.g., an anti-TCRvβ antibody) and an cytokine (e.g., an IL-2 domain) provides potent and durable single-agent anti-tumor activity in solid tumor models. In some embodiments, a multispecific molecule containing a molecule that binds to a TCRβV (e.g., an anti-TCRvβ antibody) and an cytokine (e.g., an IL-2 domain) provides potent and durable single-agent anti-tumor activity in tumor models that is dependent on expanded Vβ T cells. In some embodiments, a multispecific molecule containing a molecule that binds to a TCRβV (e.g., an anti-TCRvβ antibody) and an cytokine (e.g., an IL-2 domain) provides potent and durable single-agent anti-tumor activity in tumor models. In some embodiments, a multispecific molecule containing a molecule that binds to a TCRβV (e.g., an anti-TCRvβ antibody) and an cytokine (e.g., an IL-2 domain) induces modulation of the tumor microenvironment (TME), striking increase in TCR diversity, functional immune memory, or a combination thereof. In some embodiments, a multispecific molecule containing a molecule that binds to a TCRβV (e.g., an anti-TCRvβ antibody) and an cytokine (e.g., an IL-2 domain) remodels the adaptive immune response to solid tumors that are refractory to checkpoint inhibitor therapy. In some embodiments, a multispecific molecule containing a molecule that binds to a TCRβV (e.g., an anti-TCRvβ antibody) and an cytokine (e.g., an IL-2 domain) remodels the adaptive immune response to tumors that are refractory to checkpoint inhibitor therapy. In some embodiments, a multispecific molecule containing a molecule that binds to a TCRβV (e.g., an anti-TCRvβ antibody) and an cytokine (e.g., an IL-2 domain) remodels the adaptive immune response to solid tumors. In some embodiments, a multispecific molecule containing a molecule that binds to a TCRβV (e.g., an anti-TCRvβ antibody) and an cytokine (e.g., an IL-2 domain) remodels the adaptive immune response to tumors.

In some embodiments, stimulation of T cells with a multispecific molecule containing a molecule that binds to a TCRβV (e.g., an anti-TCRvβ antibody) and an cytokine (e.g., an IL-2 domain) results in potent expansion with adopting a novel memory phenotype, and significant boosting of antigen-specific T cells. In some embodiments, stimulation of T cells with a multispecific molecule containing a molecule that binds to a TCRβV (e.g., an anti-TCRvβ antibody) and an cytokine (e.g., an IL-2 domain) results in potent expansion with adopting a novel memory phenotype. In some embodiments, stimulation of T cells with a multispecific molecule containing a molecule that binds to a TCRβV (e.g., an anti-TCRvβ antibody) and an cytokine (e.g., an IL-2 domain) results in potent expansion with significant boosting of antigen-specific T cells. In some embodiments, a multispecific molecule containing a molecule that binds to a TCRβV (e.g., an anti-TCRvβ antibody) and an cytokine (e.g., an IL-2 domain) induces potent expansion of TILs and killing of tumors. In some embodiments, a multispecific molecule containing a molecule that binds to a TCRβV (e.g., an anti-TCRvβ antibody) and an cytokine (e.g., an IL-2 domain) induces potent expansion of TILs and killing of PD1 refractory tumors. In some embodiments, dose-related anti-tumor activity of a multispecific molecule containing a molecule that binds to a TCRβV (e.g., an anti-TCRvβ antibody) and an cytokine (e.g., an IL-2 domain) correlates with expansion of memory Vβ CD8+ T cells. In some embodiments, administration of a multispecific molecule containing a molecule that binds to a TCRβV (e.g., an anti-TCRvβ antibody) and an cytokine (e.g., an IL-2 domain) induces robust expansion of Vβ CD8+ T cells in blood, with limited cytokine release or expansion of Treg. In some embodiments, administration of a multispecific molecule containing a molecule that binds to a TCRβV (e.g., an anti-TCRvβ antibody) and an cytokine (e.g., an IL-2 domain) induces robust expansion of Vβ CD8+ T cells in blood. In some embodiments, a multispecific molecule containing a molecule that binds to a TCRβV (e.g., an anti-TCRvβ antibody) and an cytokine (e.g., an IL-2 domain) potently expands both naive and antigen-specific human T cells. In some embodiments, a multispecific molecule containing a molecule that binds to a TCRβV (e.g., an anti-TCRvβ antibody) and an cytokine (e.g., an IL-2 domain) induces potent anti-tumor activity as monotherapy, mediated by selective expansion of Vβ CD8+ memory T cells. In some embodiments, a multispecific molecule containing a molecule that binds to a TCRβV (e.g., an anti-TCRvβ antibody) and an cytokine (e.g., an IL-2 domain) provides potent anti-tumor activity for PD-1 refractory solid tumors. In some embodiments, a multispecific molecule containing a molecule that binds to a TCRβV (e.g., an anti-TCRvβ antibody) and an cytokine (e.g., an IL-2 domain) provides potent anti-tumor activity for PD-1 refractory tumors. In some embodiments, a multispecific molecule containing a molecule that binds to a TCRβV (e.g., an anti-TCRvβ antibody) and an cytokine (e.g., an IL-2 domain) provides potent anti-tumor activity for solid tumors. In some embodiments, a multispecific molecule containing a molecule that binds to a TCRβV (e.g., an anti-TCRvβ antibody) and an cytokine (e.g., an IL-2 domain) provides potent anti-tumor activity for tumors.

In some embodiments, a multispecific molecule containing a molecule that binds to a TCRβV (e.g., an anti-TCRvβ antibody) and an cytokine (e.g., an IL-2 domain) induces an atypical central-memory like phenotype in human T cells. In some embodiments, the atypical central memory (TCM)-like phenotype induced by a multispecific molecule containing a molecule that binds to a TCRβV (e.g., an anti-TCRvβ antibody) and an cytokine (e.g., an IL-2 domain) is distinct from those induced by anti-CD3s stimulation. In some embodiments, the cells stimulated by a multispecific molecule containing a molecule that binds to a TCRβV (e.g., an anti-TCRvβ antibody) and an cytokine (e.g., an IL-2 domain) show myriad surface markers associated with chronic stimulation, and are not exhausted but are highly proliferative with strong cytolytic/Tc1 effector profiles. In some embodiments, the cells stimulated by a multispecific molecule containing a molecule that binds to a TCRβV (e.g., an anti-TCRvβ antibody) and an cytokine (e.g., an IL-2 domain) show myriad surface markers associated with chronic stimulation. In some embodiments, the cells stimulated by a multispecific molecule containing a molecule that binds to a TCRβV (e.g., an anti-TCRvβ antibody) and an cytokine (e.g., an IL-2 domain) are not exhausted but are highly proliferative with strong cytolytic/Tc1 effector profiles. In some embodiments, the exemplary cytolytic/Tc1 effector profiles comprises, but are not limited to, expression of T-bet and IRF4. In some embodiments, the cells previously driven toward effector memory (T_{EM}) and effector memory T cells re-expresses CD45RA (TEMRA) states can show the phenotype of not being exhausted but being highly proliferative with strong cytolytic/Tc1 effector profiles via stimulation by a multispecific molecule containing a molecule that binds to a TCRβV (e.g., an anti-TCRvβ antibody) and an cytokine (e.g., an IL-2 domain).

Accordingly, provided herein are, inter alia, multispecific or multifunctional molecules comprising TCRβV-binding moieties as described herein (e.g., multispecific or multifunctional antibody molecules) that comprise anti-TCRβV antibody molecules, nucleic acids encoding the same, methods of producing the aforesaid molecules, pharmaceutical compositions comprising aforesaid molecules, and methods of treating a disease or disorder, e.g., cancer, using the aforesaid molecules. The antibody molecules and pharmaceutical compositions as described herein can be used (alone or in combination with other agents or therapeutic modalities) to treat, prevent and/or diagnose disorders and conditions, e.g., cancer, e.g., as described herein.

### Sequence Tables

TABLE 1
Amino acid and nucleotide sequences for murine, chimeric and humanized antibody molecules
which bind to TCRVB 6, e.g., TCRVB 6-5. The antibody molecules include murine mAb Antibody A,
and humanized mAb Antibody A-H Clones A-H.1 to A-H.85. The amino acid the heavy and light
chain CDRs, and the amino acid and nucleotide sequences of the heavy and light chain variable
regions, and the heavy and light chains are shown.
Antibody A (murine), also referred to as H131, TCRVB 6-5 binder

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 45 | HC CDR1 (Kabat) | TYYIH |
| SEQ ID NO: 46 | HC CDR2 (Kabat) | WFFPGSGNIK YNEKFKG |
| SEQ ID NO: 47 | HC CDR3 (Kabat) | SYYSYDVLDY |
| SEQ ID NO: 48 | HC CDR1 (Chothia) | GYSFTTY |
| SEQ ID NO: 49 | HC CDR2 (Chothia) | FPGSGN |
| SEQ ID NO: 50 | HC CDR3 (Chothia) | SYYSYDVLDY |
| SEQ ID NO: 1 | VH | QVQLQQSGPELVKPGTSVKISCKASGYSFTTYYIHWVKQRPG |
| | | QGLEWIGWFFPGSGNIKYNEKFKGKATLTADTSSSTAYMQL |
| | | SSLTSEESAVYFCAGSYYSYDVLDYWGHGTTLTVSS |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 51 | LC CDR1 (Kabat) | KASQNVGINVV |
| SEQ ID NO: 52 | LC CDR2 (Kabat) | SSSHRYS |
| SEQ ID NO: 53 | LC CDR3 (Kabat) | QQFKSYPLT |
| SEQ ID NO: 54 | LC CDR1 (Chothia) | KASQNVGINVV |
| SEQ ID NO: 55 | LC CDR2 (Chothia) | SSSHRYS |
| SEQ ID NO: 56 | LC CDR3 (Chothia) | QQFKSYPLT |
| SEQ ID NO: 2 | VL | DILMTQSQKFMSTSLGDRVSVSCKASQNVGINVVWHQQKPG |
| | | QSPKALIYSSSHRYSGVPDRFTGSGSGTDFTLTINNVQSEDLA |
| | | EYFCQQFKSYPLTFGAGTKLELK |

Antibody A humanized (A-H antibody), TCRVB 6-5 binder
A-H.1 antibody (also referred to as BEIM1709)

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 9 | VH | QVQLVQSGAEVKKPGSSVKVSCKASGYSFTTYYIHWVRQAP |
| | | GQGLEWMGWFFPGSGNIKYNEKFKGRVTITADTSTSTAYME |
| | | LSSLRSEDTAVYYCAGSYYSYDVLDYWGQGTTVTVSS |
| SEQ ID NO: 12 | DNA VH | CAGGTGCAGCTGGTTCAGTCTGGCGCCGAAGTGAAGAAA |
| | | CCTGGCTCCTCCGTGAAGGTGTCCTGCAAGGCTTCCGGCT |
| | | ACTCCTTCACCACCTACTACATCCACTGGGTCCGACAGGC |
| | | CCCTGGACAAGGATTGGAATGGATGGGCTGGTTCTTCCCC |
| | | GGCTCCGGCAACATCAAGTACAACGAGAAGTTCAAGGGC |
| | | CGCGTGACCATCACCGCCGACACCTCTACCTCTACCGCCT |
| | | ACATGGAACTGTCCAGCCTGAGATCTGAGGACACCGCCGT |
| | | GTACTACTGCGCCGGCTCCTACTACTCTTACGACGTGCTG |
| | | GATTACTGGGGCCAGGGCACCACAGTGACAGTGTCCTCT |
| SEQ ID NO: 69 | VH-IgM constant delta | METDTLLLWVLLLWVPGSTGQVQLVQSGAEVKKPGSSVKV |
| | CDC | SCKASGYSFTTYYIHWVRQAPGQGLEWMGWFFPGSGNIKY |
| | | NEKFKGRVTITADTSTSTAYMELSSLRSEDTAVYYCAGSYYS |
| | | YDVLDYWGQGTTVTVSSGSASAPTLFPLVSCENSPSDTSSVA |
| | | VGCLAQDFLPDSITFSWKYKNNSDISSTRGFPSVLRGGKYAA |
| | | TSQVLLPSKDVMQGTDEHVVCKVQHPNGNKEKNVPLPVIAE |
| | | LPPKVSVFVPPRDGFFGNPRKSKLICQATGFSPRQIQVSWLRE |
| | | GKQVGSGVTTDQVQAEAKESGPTTYKVTSTLTIKESDWLGQ |
| | | SMFTCRVDHRGLTFQQNASSMCVPDQDTAIRVFAIPPSFASIF |
| | | LTKSTKLTCLVTDLTTYDSVTISWTRQNGEAVKTHTNISESH |
| | | PNATFSAVGEASICEDDWNSGERFTCTVTHTDLASSLKQTIS |
| | | RPKGVALHRPDVYLLPPAREQLNLRESATITCLVTGFSPADV |
| | | FVQWMQRGQPLSPEKYVTSAPMPEPQAPGRYFAHSILTVSEE |
| | | EWNTGETYTCVVAHEALPNRVTERTVDKSTGKPTLYNVSLV |
| | | MSDTAGTCY |
| SEQ ID NO: 70 | VH-IgGA1 | METDTLLLWVLLLWVPGSTGQVQLVQSGAEVKKPGSSVKV |
| | | SCKASGYSFTTYYIHWVRQAPGQGLEWMGWFFPGSGNIKY |
| | | NEKFKGRVTITADTSTSTAYMELSSLRSEDTAVYYCAGSYYS |
| | | YDVLDYWGQGTTVTVSSASPTSPKVFPLSLCSTQPDGNVVIA |
| | | CLVQGFFPQEPLSVTWSESGQGVTARNFPPSQDASGDLYTTS |
| | | SQLTLPATQCLAGKSVTCHVKHYTNPSQDVTVPCPVPSTPPT |
| | | PSPSTPPTPSPSCCHPRLSLHRPALEDLLLGSEANLTCTLTGLR |
| | | DASGVTFTWTPSSGKSAVQGPPERDLCGCYSVSSVLPGCAEP |
| | | WNHGKTFTCTAAYPESKTPLTATLSKSGNTFRPEVHLLPPPS |
| | | EELALNELVILTCLARGFSPKDVLVRWLQGSQELPREKYLT |
| | | WASRQEPSQGTTTFAVTSILRVAAEDWKKGDTFSCMVGHEA |
| | | LPLAFTQKTIDRLAGKPTHVNVSVVMAEVDGTCY |
| SEQ ID NO: 71 | VH-IgGA2 | METDTLLLWVLLLWVPGSTGQVQLVQSGAEVKKPGSSVKV |
| | | SCKASGYSFTTYYIHWVRQAPGQGLEWMGWFFPGSGNIKY |
| | | NEKFKGRVTITADTSTSTAYMELSSLRSEDTAVYYCAGSYYS |
| | | YDVLDYWGQGTTVTVSSASPTSPKVFPLSLDSTPQDGNVVV |
| | | ACLVQGFFPQEPLSVTWSESGQNVTARNFPPSQDASGDLYTT |
| | | SSQLTLPATQCPDGKSVTCHVKHYTNSSQDVTVPCRVPPPPP |
| | | CCHPRLSLHRPALEDLLLGSEANLTCTLTGLRDASGATFTWT |
| | | PSSGKSAVQGPPERDLCGCYSVSSVLPGCAQPWNHGETFTCT |
| | | AAHPELKTPLTANITKSGNTFRPEVHLLPPPSEELALNELVTL |
| | | TCLARGFSPKDVLVRWLQGSQELPREKYLTWASRQEPSQGT |
| | | TTYAVTSILRVAAEDWKKGETFSCMVGHEALPLAFTQKTID |
| | | RMAGKPTHINVSVVMAEADGTCY |
| SEQ ID NO: | Heavy chain | METDTLLLWVLLLWVPGSTGQVQLVQSGAEVKKPGSSVKV |
| 3278 | | SCKASGYSFTTYYIHWVRQAPGQGLEWMGWFFPGSGNIKY |
| | | NEKFKGRVTITADTSTSTAYMELSSLRSEDTAVYYCAGSYYS |
| | | YDVLDYWGQGTTVTVSSASTKGPSVFPLAPSSKSTSGGTAA |
| | | LGCLVKDYFPEPVTVSWNSGALTSGVHTFPAVLQSSGLYSLS |
| | | SVVTVPSSSLGTQTYICNVNHKPSNTKVDKRVEPKSCDKTHT |
| | | CPPCPAPELLGGPSVFLFPPKPKDTLMISRTPEVTCVVVDVSH |
| | | EDPEVKFNWYVDGVEVHNAKTKPREEQYNSTYRVVSVLTV |
| | | LHQDWLNGKEYKCKVSNKALPAPIEKTISKAKGQPREPQVY |
| | | TLPPSREEMTKNQVSLTCLVKGFYPSDIAVEWESNGQPENNY |
| | | KTTPPVLDSDGSFFLYSKLTVDKSRWQQGNVFSCSVMHEAL |
| | | HNHYTQKSLSLSPGK |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 10 | VL | DIQMTQSPSFLSASVGDRVTITCKASQNVGINVVWHQQKPG |
| | | KAPKALIYSSSHRYSGVPSRFSGSGSGTEFTLTISSLQPEDFAT |
| | | YFCQQFKSYPLTFGQGTKLEIK |
| SEQ ID NO: 13 | DNA VL | GACATCCAGATGACCCAGTCTCCATCCTTCCTGTCCGCCTC |
| | | TGTGGGCGACAGAGTGACCATCACATGCAAGGCCTCTCAG |
| | | AACGTGGGCATCAACGTCGTGTGGCACCAGCAGAAGCCT |
| | | GGCAAGGCTCCTAAGGCTCTGATCTACTCCTCCAGCCACC |
| | | GGTACTCTGGCGTGCCCTCTAGATTTTCCGGCTCTGGCTCT |
| | | GGCACCGAGTTTACCCTGACAATCTCCAGCCTGCAGCCTG |
| | | AGGACTTCGCCACCTACTTTTGCCAGCAGTTCAAGAGCTA |
| | | CCCTCTGACCTTTGGCCAGGGCACCAAGCTGGAAATCAAG |
| SEQ ID NO: 72 | VL and kappa constant | METDTLLLWVLLLWVPGSTGDIQMTQSPSFLSASVGDRVTIT |
| | region/light chain | CKASQNVGINVVWHQQKPGKAPKALIYSSSHRYSGVPSRFS |
| | | GSGSGTEFTLTISSLQPEDFATYFCQQFKSYPLTFGQGTKLEIK |
| | | RTVAAPSVFIFPPSDEQLKSGTASVVCLLNNFYPREAKVQWK |
| | | VDNALQSGNSQESVTEQDSKDSTYSLSSTLTLSKADYEKHK |
| | | VYACEVTHQGLSSPVTKSFNRGEC |

A-H.2 antibody (also referred to as BHM1710)

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 9 | VH | QVQLVQSGAEVKKPGSSVKVSCKASGYSFTTYYIHWVRQAP |
| | | GQGLEWMGWFFPGSGNIKYNEKFKGRVTITADTSTSTAYME |
| | | LSSLRSEDTAVYYCAGSYYSYDVLDYWGQGTTVTVSS |
| SEQ ID NO: 12 | DNA VH | CAGGTGCAGCTGGTTCAGTCTGGCGCCGAAGTGAAGAAA |
| | | CCTGGCTCCTCCGTGAAGGTGTCCTGCAAGGCTTCCGGCT |
| | | ACTCCTTCACCACCTACTACATCCACTGGGTCCGACAGGC |
| | | CCCTGGACAAGGATTGGAATGGATGGGCTGGTTCTTCCCC |
| | | GGCTCCGGCAACATCAAGTACAACGAGAAGTTCAAGGGC |
| | | CGCGTGACCATCACCGCCGACACCTCTACCTCTACCGCCT |
| | | ACATGGAACTGTCCAGCCTGAGATCTGAGGACACCGCCGT |
| | | GTACTACTGCGCCGGCTCCTACTACTCTTACGACGTGCTG |
| | | GATTACTGGGGCCAGGGCACCACAGTGACAGTGTCCTCT |
| SEQ ID NO: | Heavy chain | METDTLLLWVLLLWVPGSTGQVQLVQSGAEVKKPGSSVKV |
| 3278 | | SCKASGYSFTTYYIHWVRQAPGQGLEWMGWFFPGSGNIKY |
| | | NEKFKGRVTITADTSTSTAYMELSSLRSEDTAVYYCAGSYYS |
| | | YDVLDYWGQGTTVTVSSASTKGPSVFPLAPSSKSTSGGTAA |
| | | LGCLVKDYFPEPVTVSWNSGALTSGVHTFPAVLQSSGLYSLS |
| | | SVVTVPSSSLGTQTYICNVNHKPSNTKVDKRVEPKSCDKTHT |
| | | CPPCPAPELLGGPSVFLFPPKPKDTLMISRTPEVTCVVVDVSH |
| | | EDPEVKFNWYVDGVEVHNAKTKPREEQYNSTYRVVSVLTV |
| | | LHQDWLNGKEYKCKVSNKALPAPIEKTISKAKGQPREPQVY |
| | | TLPPSREEMTKNQVSLTCLVKGFYPSDIAVEWESNGQPENNY |
| | | KTTPPVLDSDGSFFLYSKLTVDKSRWQQGNVFSCSVMHEAL |
| | | HNHYTQKSLSLSPGK |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 11 | VL | DIQMTQSPSSLSASVGDRVTITCKASQNVGINVVWHQQKPG |
| | | KVPKALIYSSSHRYSGVPSRFSGSGSGTDFTLTISSLQPEDVAT |
| | | YFCQQFKSYPLTFGQGTKLEIK |
| SEQ ID NO: 14 | DNA VL | GACATCCAGATGACCCAGTCTCCATCCTCTCTGTCCGCCTC |
| | | TGTGGGCGACAGAGTGACCATCACATGCAAGGCCTCTCAG |
| | | AACGTGGGCATCAACGTCGTGTGGCACCAGCAGAAACCT |
| | | GGCAAGGTGCCCAAGGCTCTGATCTACTCCTCCAGCCACA |
| | | GATACTCCGGCGTGCCCTCTAGATTCTCCGGCTCTGGCTCT |
| | | GGCACCGACTTTACCCTGACAATCTCCAGCCTGCAGCCTG |
| | | AGGACGTGGCCACCTACTTTTGCCAGCAGTTCAAGAGCTA |
| | | CCCTCTGACCTTTGGCCAGGGCACCAAGCTGGAAATCAAG |
| SEQ ID NO: | Light chain | METDTLLLWVLLLWVPGSTGDIQMTQSPSSLSASVGDRVTIT |
| 3279 | | CKASQNVGINVVWHQQKPGKVPKALIYSSSHRYSGVPSRFS |
| | | GSGSGTDFTLTISSLQPEDVATYFCQQFKSYPLTFGQGTKLEI |
| | | KRTVAAPSVFIFPPSDEQLKSGTASVVCLLNNFYPREAKVQW |
| | | KVDNALQSGNSQESVTEQDSKDSTYSLSSTLTLSKADYEKH |
| | | KVYACEVTHQGLSSPVTKSFNRGEC |

A-H.3 antibody

| | | |
|---|---|---|
| SEQ ID NO: 80 | VH + VL | QVQLVQSGAEVKKPGSSVKVSCKASGTDFKLTYIHWVRQAP |
| | | GQGLEWMGRVSPGSGNTKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVGDRVTITCK |
| | | ASQNVEDRVAWYQQKPGKAPKALIYSSSHRYKGVPSRFSGS |
| | | GSGTEFTLTISSLQPEDFATYFCQQFKSYPLTFGQGTKLEIK |
| SEQ ID NO: 81 | VL | DIQMTQSPSFLSASVGDRVTITCKASQNVEDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 82 | VH | QVQLVQSGAEVKKPGSSVKVSCKASGTDFKLTYIHWVRQAP |
| | | GQGLEWMGRVSPGSGNTKYNEKFKGRVTITADTSTSTAYM |
| | | ELSSLRSEDTAVYYCAGSYYSYDVLDYWGQGTTVTVSS |

A-H.4

| | | |
|---|---|---|
| SEQ ID NO: 83 | VH + VL | QVQLVQSGAEVKKPGSSVKVSCKASGTDFDKIYIHWVRQAP |
| | | GQGLEWMGRISAGSGNVKYNEKFKGRVTITADTSTSTAYME |
| | | LSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVEDRVAWYQQKPGKAPKALIYSSSHRYKGVPSRFSGSG |
| | | SGTEFTLTISSLQPEDFATYFCQQFKSYPLTFGQGTKLEIK |
| SEQ ID NO: 84 | VL | DIQMTQSPSFLSASVQDRVTITCKASQNVEDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 85 | VH | QVQLVQSGAEVKKPGSSVKVSCKASGTDFDKIYIHWVRQAP |
| | | GQGLEWMGRISAGSGNVKYNEKFKGRVTITADTSTSTAYME |
| | | LSSLRSEDTAVYYCAGSYYSYDVLDYWGQGTTVTVSS |

A-H.5

| | | |
|---|---|---|
| SEQ ID NO: 86 | VH + VL | QVQLVQSGAEVKKPGSSVKVSCKASGHDFRDFYIHWVRQA |
| | | PGQGLEWMGRVYPGSGSYRYNEKFKGRVTITADTSTSTAY |
| | | MELSSLRSEDTAVYYCAGSYYSYDVLDYWGQGTTVTVSSG |
| | | GGGSGGGGSGGGGSGGGGSDIQMTQSPSFLSASVQDRVTITC |
| | | KASQNVDDRVAWYQQKPGKAPKALIYSSSHRYKGVPSRFSG |
| | | SGSGTEFTLTISSLQPEDFATYFCQQFKSYPLTFGQGTKLEIK |
| SEQ ID NO: 87 | VL | DIQMTQSPSFLSASVGDRVTITCKASQNVDDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 88 | VH | QVQLVQSGAEVKKPGSSVKVSCKASGHDFRDFYIHWVRQA |
| | | PGQGLEWMGRVYPGSGSYRYNEKFKGRVTITADTSTSTAY |
| | | MELSSLRSEDTAVYYCAGSYYSYDVLDYWGQGTTVTVSS |

A-H.6

| | | |
|---|---|---|
| SEQ ID NO: 89 | VH + VL | QVQLVQSGAEVKKPGSSVKVSCKASGHDFKLTYIHWVRQA |
| | | PGQGLEWMGRISAGSGNVKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVGDRVTTTCK |
| | | ASQNVDNRVAWYQQKPGKAPKALIYSSSHRYKGVPSRFSGS |
| | | GSGTEFTLTISSLQPEDFATYFCQQFKSYPLTFGQGTKLEIK |
| SEQ ID NO: 90 | VL | DIQMTQSPSFLSASVGDRVTITCKASQNVDNRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 91 | VH | QVQLVQSGAEVKKPGSSVKVSCKASGHDFKLTYIHWVRQA |
| | | PGQGLEWMGRISAGSGNVKYNEKFKGRVTITADTSTSTAYM |
| | | ELSSLRSEDTAVYYCAGSYYSYDVLDYWGQGTTVTVSS |

A-H.7

| | | |
|---|---|---|
| SEQ ID NO: 92 | VH + VL | QVQLVQSGAEVKKPGSSVKVSCKASGTDFKLTYIHWVRQAP |
| | | GQGLEWMGRIFPGSGNVKYNEKFKGRVTITADTSTSTAYME |
| | | LSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVENKVAWHQQKPGKAPKALIYSSSHRYKGVPSRFSGSG |
| | | SGTEFTLTISSLQPEDFATYFCQQFKSYPLTFGQGTKLEIK |
| SEQ ID NO: 93 | VL | DIQMTQSPSFLSASVGDRVTITCKASQNVENKVAWHQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 94 | VH | QVQLVQSGAEVKKPGSSVKVSCKASGTDFKLTYIHWVRQAP |
| | | GQGLEWMGRIFPGSGNVKYNEKFKGRVTITADTSTSTAYME |
| | | LSSLRSEDTAVYYCAGSYYSYDVLDYWGQGTTVTVSS |

A-H.8

| | | |
|---|---|---|
| SEQ ID NO: 95 | VH + VL | QVQLVQSGAEVKKPGSSVKVSCKASGTDFDKIYIHWVRQAP |
| | | GQGLEWMGRIFAGSGNTKYNEKFKGRVTITADTSTSTAYME |
| | | LSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVDDRVAWYQQKPGKAPKALIYSSSHRYKGVPSRFSGSG |
| | | SGTEFTLTISSLQPEDFATYFCQQFKSYPLTFGQGTKLEIK |
| SEQ ID NO: 96 | VL | DIQMTQSPSFLSASVGDRVTITCKASQNVDDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 97 | VH | QVQLVQSGAEVKKPGSSVKVSCKASGTDFDKIYIHWVRQAP |
| | | GQGLEWMGRIFAGSGNTKYNEKFKGRVTITADTSTSTAYME |
| | | LSSLRSEDTAVYYCAGSYYSYDVLDYWGQGTTVTVSS |

A-H.9

| | | |
|---|---|---|
| SEQ ID NO: 98 | VH + VL | QVQLVQSGAEVKKPGSSVKVSCKASGHDFDKFYIHWVRQA |
| | | PGQGLEWMGRVSAGSGNVKYNEKFKGRVTITADTSTSTAY |
| | | MELSSLRSEDTAVYYCAGSYYSYDVLDYWGQGTTVTVSSG |
| | | GGGSGGGGSGGGGSGGGGSDIQMTQSPSFLSASVGDRVTITC |
| | | KASQNVGNRVAWYQQKPGKAPKALIYSSSHRYSGVPSRFSG |
| | | SGSGTEFTLTISSLQPEDFATYFCQQFKSYPLTFGQGTKLEIK |
| SEQ ID NO: 99 | VL | DIQMTQSPSFLSASVGDRVTITCKASQNVGNRVAWYQQKPG |
| | | KAPKALIYSSSHRYSGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQA |
| 100 | | PGQGLEWMGRVSAGSGNVKYNEKFKGRVTITADTSTSTAY |
| | | MELSSLRSEDTAVYYCAGSYYSYDVLDYWGQGTTVTVSS |

A-H.10

| | | |
|---|---|---|
| SEQ ID NO: | VH + VL | QVQLVQSGAEVKKPGSSVKVSCKASGHDFDKFYIHWVRQA |
| 101 | | PGQGLEWMGRIFAGSGNVKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVGDRVTITCK |
| | | ASQNVGDRVAWYQQKPGKAPKALIYSSSHRYKGVPSRFSGS |
| | | GSGTEFTLTISSLQPEDFATYFCQQFKSYPLTFGQGTKLEIKs |
| SEQ ID NO: | VL | DIQMTQSPSFLSASVGDRVTITCKASQNVGDRVAWYQQKPG |
| 102 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQA |
| 103 | | PGQGLEWMGRIFAGSGNVKYNEKFKGRVTITADTSTSTAYM |
| | | ELSSLRSEDTAVYYCAGSYYSYDVLDYWGQGTTVTVSS |

A-H.11

| | | |
|---|---|---|
| SEQ ID NO: | VH + VL | QVQLVQSGAEVKKPGSSVKVSCKASGTDFKLTYIHWVRQAP |
| 104 | | GQGLEWMGRVSPGSGNVKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVGDRVTITCK |
| | | ASQNVGDRVAWYQQKPGKAPKALIYSSSHRYKGVPSRFSGS |
| | | GSGTEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 105 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 106 | | GQGLEWMGRVSPGSGNVKYNEKFKGRVTITADTSTSTAYM |
| | | ELSSLRSEDTAVYYCAGSYYSYDVLDYWGQGTTVTVSS |

A-H.12

| | | |
|---|---|---|
| SEQ ID NO: | VH + VL | QVQLVQSGAEVKKPGSSVKVSCKASGTDFDKIYIHWVRQAP |
| 107 | | GQGLEWMGRVSAGSGNTKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVGDRVTITCK |
| | | ASQNVGNRVAWYQQKPGKAPKALIYSSSHRYKGVPSRFSGS |
| | | GSGTEFTLTISSLQPEDFATYFCQQFKSYPLTFGQGTKLEIK |
| SEQ ID NO: | VI | DIQMTQSPSELSASVQDRVTITCKASQNVGNRVAWYQQKPG |
| 108 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 109 | | GQGLEWMGRVSAGSGNTKYNEKFKGRVTITADTSTSTAYM |
| | | ELSSLRSEDTAVYYCAGSYYSYDVLDYWGQGTTVTVSS |

A-H.13, also referred to as A-H.69

| | | |
|---|---|---|
| SEQ ID NO: | VH + VL | QVQLVQSGAEVKKPGSSVKVSCKASGTDFKLTYIHWVRQAP |
| 110 | | GQGLEWMGRIFPGSGNVKYNEKFKGRVTITADTSTSTAYME |
| | | LSSLRSEDTAVYYCAGSYYSYDVLDYWGQGTTVTVSSGGG |
| | | GSGGGGSGGGGSQGGGSDIQMTQSPSFLSASVGDRVTITCKA |
| | | SQNVDNRVAWYQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVDNRVAWYQQKPG |
| 111 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 112 | | GQGLEWMGRIFPGSGNVKYNEKFKGRVTITADTSTSTAYME |
| | | LSSLRSEDTAVYYCAGSYYSYDVLDYWGQGTTVTVSS |

A-H.14

| | | |
|---|---|---|
| SEQ ID NO: | VH + VL | QVQLVQSGAEVKKPGSSVKVSCKASGTDFDKIYIHWVRQAP |
| 113 | | GQGLEWMGRISAGSGNTKYNEKFKGRVTITADTSTSTAYME |
| | | LSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVDDRVAWYQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 114 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 115 | | GQGLEWMGRISAGSGNTKYNEKFKGRVTITADTSTSTAYME |
| | | LSSLRSEDTAVYYCAGSYYSYDVLDYWGQGTTVTVSS |

A-H.15

| | | |
|---|---|---|
| SEQ ID NO: | VH + VL | QVQLVQSGAEVKKPGSSVKVSCKASGTDFRLTYIHWVRQAP |
| 116 | | GQGLEWMGRVSPGSGNTKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVQDRVTITQK |
| | | ASQNVDNKVAWHQQKPGKAPKALIYSSSHRYKGVPSRFSGS |
| | | GSGTEFTLTISSLQPEDFATYFCQQFKSYPLTFGQGTKLEIK |
| SEQ ID NO: | VL | DIQMTQSPSFLSASVGDRVTITCKASQNVDNKVAWHQQKPG |
| 117 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFRLTYIHWVRQAP |
| 118 | | GQGLEWMGRVSPGSGNTKYNEKFKGRVTITADTSTSTAYM |
| | | ELSSLRSEDTAVYYCAGSYYSYDVLDYWGQGTTVTVSS |

A-H.16

| | | |
|---|---|---|
| SEQ ID NO: | VH + VL | QVQLVQSGAEVKKPGSSVKVSCKASGGTFRLTYIHWVRQAP |
| 119 | | GQGLEWMGRVYPGSQNTKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVGDRVTITCK |
| | | ASQNVDDRVAWYQQKPGKAPKALIYSSSHRYKGVPSRFSGS |
| | | GSGTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 120 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFRLTYIHWVRQAP |
| 121 | | GQGLEWMGRVYPGSGNTKYNEKFKGRVTITADTSTSTAYM |
| | | ELSSLRSEDTAVYYCAGSYYSYDVLDYWGQGTTVTVSS |

A-H.17

| | | |
|---|---|---|
| SEQ ID NO: | VH + VL | QVQLVQSGAEVKKPGSSVKVSCKASGTDFRLTYIHWVRQAP |
| 122 | | GQGLEWMGRIFPGSGNTKYNEKFKGRVTITADTSTSTAYME |
| | | LSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVDDRVAWYQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 123 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFRLTYIHWVRQAP |
| 124 | | GQGLEWMGRIFPGSGNTKYNEKFKGRVTITADTSTSTAYME |
| | | LSSLRSEDTAVYYCAGSYYSYDVLDYWGQGTTVTVSS |

A-H.18

| | | |
|---|---|---|
| SEQ ID NO: | VH + VL | QVQLVQSGAEVKKPGSSVKVSCKASGTDFKLTYIHWVRQAP |
| 125 | | GQGLEWMGRIFPGSGNVKYNEKFKGRVTITADTSTSTAYME |
| | | LSSLRSEDTAVYYCAGSYYSYDVLDYWGQGTTVTVSSQGG |
| | | GSGGGGSGGGGSGGGGSDIQMTQSPSFLSASVGDRVTITCKA |
| | | SQNVEDRVAWYQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVEDRVAWYQQKPG |
| 126 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 127 | | GQGLEWMGRIFPGSGNVKYNEKFKGRVTITADTSTSTAYME |
| | | LSSLRSEDTAVYYCAGSYYSYDVLDYWGQGTTVTVSS |

A-H.19

| | | |
|---|---|---|
| SEQ ID NO: | VH + VL | QVQLVQSGAEVKKPGSSVKVSCKASGGTFRLTYIHWVRQAP |
| 128 | | GQGLEWMGRISAGSGNVKYNEKFKGRVTITADTSTSTAYME |
| | | LSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVGDRVAWYQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 129 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFRLTYIHWVRQAP |
| 130 | | GQGLEWMGRISAGSGNVKYNEKFKGRVTITADTSTSTAYME |
| | | LSSLRSEDTAVYYCAGSYYSYDVLDYWGQGTTVTVSS |

A-H.20

| | | |
|---|---|---|
| SEQ ID NO: | VH + VL | QVQLVQSGAEVKKPGSSVKVSCKASGGTFDKTYIHWVRQA |
| 131 | | PGQGLEWMGRISAGSGNTKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVGDRVTITCK |
| | | ASQNVDDRVAWYQQKPGKAPKALIYSSSHRYKGVPSRFSGS |
| | | GSGTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 132 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFDKTYIHWVRQA |
| 133 | | PGQGLEWMGRISAGSGNTKYNEKFKGRVTITADTSTSTAYM |
| | | ELSSLRSEDTAVYYCAGSYYSYDVLDYWGQGTTVTVSS |

A-H.21

| | | |
|---|---|---|
| SEQ ID NO: | VH + VL | QVQLVQSGAEVKKPGSSVKVSCKASGHDFDKFYIHWVRQA |
| 134 | | PGQGLEWMGRISAGSGNTKYNEKFKGRVTITADTSTSTAYM |
| | | ELSSLRSEDTAVYYCAGSYYSYDVLDYWGQGTTVTVSSGG |
| | | GGSGGGGGGGGSGGGGSDIQMTQSPSFLSASVGDRVTITCK |
| | | ASQNVDDRVAWYQQKPGKAPKALIYSSSHRYKGVPSRFSGS |
| | | GSGTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 135 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQA |
| 136 | | PGQGLEWMGRISAGSGNTKYNEKFKGRVTITADTSTSTAYM |
| | | ELSSLRSEDTAVYYCAGSYYSYDVLDYWGQGTTVTVSS |

A-H.22

| | | |
|---|---|---|
| SEQ ID NO: | VH + VL | QVQLVQSGAEVKKPGSSVKVSCKASGTDFKLTYIHWVRQAP |
| 137 | | GQGLEWMGRISAGSGNVKYNEKFKGRVTITADTSTSTAYME |
| | | LSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVDNKVAWHQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVDNKVAWHQQKPG |
| 138 | | KAPKALIYSSSHRYKGVPSRFSGSGSQTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 139 | | GQGLEWMGRISAGSGNVKYNEKFKGRVTITADTSTSTAYME |
| | | LSSLRSEDTAVYYCAGSYYSYDVLDYWGQGTTVTVSS |

A-H.23

| | | |
|---|---|---|
| SEQ ID NO: | VH + VL | QVQLVQSGAEVKKPGSSVKVSCKASGHDFRLTYIHWVRQAP |
| 140 | | GQGLEWMGRISAGSGNVKYNEKFKGRVTITADTSTSTAYME |
| | | LSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVADRVAWYQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVADRVAWYQQKPG |
| 141 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFRLTYIHWVRQAP |
| 142 | | GQGLEWMGRISAGSGNVKYNEKFKGRVTITADTSTSTAYME |
| | | LSSLRSEDTAVYYCAGSYYSYDVLDYWGQGTTVTVSS |

A-H.24

| | | |
|---|---|---|
| SEQ ID NO: | VH + VL | QVQLVQSGAEVKKPGSSVKVSCKASGHDFHLWYIHWVRQA |
| 143 | | PGQGLEWMGRVSAGSGNVKYNEKFKGRVTITADTSTSTAY |
| | | MELSSLRSEDTAVYYCAGSYYSYDVLDYWGQGTTVTVSSG |
| | | GGGSGGGGSGGGGSGGGGSDIQMTQSPSFLSASVGDRVTITC |
| | | KASQNVDNKVAWHQQKPGKAPKALIYSSSHRYKGVPSRFS |
| | | GSGSGTEFTLTISSLQPEDFATYFCQQFKSYPLTFGQGTKLEIK |
| SEQ ID NO: | VL | DIQMTQSPSFLSASVGDRVTITCKASQNVDNKVAWHQQKPG |
| 144 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFHLWYIHWVRQA |
| 145 | | PGQGLEWMGRVSAGSGNVKYNEKFKGRVTITADTSTSTAY |
| | | MELSSLRSEDTAVYYCAGSYYSYDVLDYWGQGTTVTVSS |

A-H.25

| | | |
|---|---|---|
| SEQ ID NO: | VH + VL | QVQLVQSGAEVKKPGSSVKVSCKASGHDFHLWYIHWVRQA |
| 146 | | PGQGLEWMGRVFAGSGNTKYNEKFKGRVTITADTSTSTAY |
| | | MELSSLRSEDTAVYYCAGSYYSYDVLDYWGQGTTVTVSSG |
| | | GGGSGGGGSGGGGSGGGGSDIQMTQSPSFLSASVQDRVTITC |
| | | KASQNVEDKVAWYQQKPGKAPKALIYSSSHRYKGVPSRFSG |
| | | SGSGTEFTLTISSLQPEDFATYFCQQFKSYPLTFGQGTKLEIK |
| SEQ ID NO: | VL | DIQMTQSPSFLSASVGDRVTITCKASQNVEDKVAWYQQKPG |
| 147 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFHLWYIHWVRQA |
| 148 | | PGQGLEWMGRVFAGSGNTKYNEKFKGRVTITADTSTSTAY |
| | | MELSSLRSEDTAVYYCAGSYYSYDVLDYWGQGTTVTVSS |

A-H.26

| | | |
|---|---|---|
| SEQ ID NO: | VH + VL | QVQLVQSGAEVKKPGSSVKVSCKASGTDFKLTYIHWVRQAP |
| 149 | | GQGLEWMGRIFPGSGNTKYNEKFKGRVTITADTSTSTAYME |
| | | LSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVDDRVAWYQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 150 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 151 | | GQGLEWMGRIFPGSGNTKYNEKFKGRVTITADTSTSTAYME |
| | | LSSLRSEDTAVYYCAGSYYSYDVLDYWGQGTTVTVSS |

A-H.27

| | | |
|---|---|---|
| SEQ ID NO: | VH + VL | QVQLVQSGAEVKKPGSSVKVSCKASGTDFKLTYIHWVRQAP |
| 153 | | GQGLEWMGRVSAGSGNVKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVGDRVTITCK |
| | | ASQNVGNRVAWYQQKPGKAPKALIYSSSHRYKGVPSRFSGS |
| | | GSGTEFTLTISSLQPEDFATYFCQQFKSYPLTFGQGTKLEIK |
| SEQ ID NO: | VL | DIQMTQSPSFLSASVGDRVTITCKASQNVGNRVAWYQQKPG |
| 154 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 155 | | GQGLEWMGRVSAGSGNVKYNEKFKGRVTITADTSTSTAYM |
| | | ELSSLRSEDTAVYYCAGSYYSYDVLDYWGQGTTVTVSS |

A-H.28

| | | |
|---|---|---|
| SEQ ID NO: | VH + VL | QVQLVQSGAEVKKPGSSVKVSCKASGTDFKLTYIHWVRQAP |
| 156 | | GQGLEWMGRISPGSGNTKYNEKFKGRVTITADTSTSTAYME |
| | | LSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVGDRVAWYQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 157 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 158 | | GQGLEWMGRISPGSGNTKYNEKFKGRVTITADTSTSTAYME |
| | | LSSLRSEDTAVYYCAGSYYSYDVLDYWGQGTTVTVSS |

A-H.29

| | | |
|---|---|---|
| SEQ ID NO: | VH + VL | QVQLVQSGAEVKKPGSSVKVSCKASGHDFHLWYIHWVRQA |
| 159 | | PGQGLEWMGRISPGSGNVKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVGDRVTITCK |
| | | ASQNVGDRVAWHQQKPGKAPKALIYSSSHRYKGVPSRFSGS |
| | | GSGTEFTLTISSLQPEDFATYFCQQFKSYPLTFGQGTKLEIK |
| SEQ ID NO: | VL | DIQMTQSPSFLSASVGDRVTITCKASQNVGDRVAWHQQKPG |
| 160 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFHLWYIHWVRQA |
| 161 | | PGQGLEWMGRISPGSGNVKYNEKFKGRVTITADTSTSTAYM |
| | | ELSSLRSEDTAVYYCAGSYYSYDVLDYWGQGTTVTVSS |

A-H.31

| | | |
|---|---|---|
| SEQ ID NO: | VH + VL | QVQLVQSGAEVKKPGSSVKVSCKASGHDFKLTYIHWVRQA |
| 162 | | PGQGLEWMGRISAGSGNVKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVGDRVTITCK |
| | | ASQNVDDRVAWYQQKPGKAPKALIYSSSHRYKGVPSRFSGS |
| | | GSGTEFTLTISSLQPEDFATYFCQQFKSYPLTFGQGTKLEIK |
| SEQ ID NO: | VL | DIQMTQSPSELSASVGDRVTITCKASQNVDDRVAWYQQKPG |
| 163 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFKLTYIHWVRQA |
| 164 | | PGQGLEWMGRISAGSGNVKYNEKFKGRVTITADTSTSTAYM |
| | | ELSSLRSEDTAVYYCAGSYYSYDVLDYWGQGTTVTVSS |

A-H.31

| | | |
|---|---|---|
| SEQ ID NO: | VH + VL | QVQLVQSGAEVKKPGSSVKVSCKASGTDFHLWYIHWVRQA |
| 165 | | PGQGLEWMGRVFAGSGSYRYNEKFKGRVTITADTSTSTAY |
| | | MELSSLRSEDTAVYYCAGSYYSYDVLDYWGQGTTVTVSSG |
| | | GGGSGGGQSGGGGSGGGGSDIQMTQSPSFLSASVQDRVTITC |
| | | KASQNVDDRVAWYQQKPGKAPKALIYSSSHRYKGVPSRFSG |
| | | SGSGTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 166 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFHLWYIHWVRQA |
| 167 | | PGQGLEWMGRVFAGSGSYRYNEKFKGRVTITADTSTSTAY |
| | | MELSSLRSEDTAVYYCAGSYYSYDVLDYWGQGTTVTVSS |

A-H.32

| | | |
|---|---|---|
| SEQ ID NO: | VH + VL | QVQLVQSGAEVKKPGSSVKVSCKASGTDFDKIYIHWVRQAP |
| 168 | | GQGLEWMGRISAGSGNTKYNEKFKGRVTITADTSTSTAYME |
| | | LSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVADRVAWYQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVADRVAWYQQKPG |
| 169 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 170 | | GQGLEWMGRISAGSGNTKYNEKFKGRVTITADTSTSTAYME |
| | | LSSLRSEDTAVYYCAGSYYSYDVLDYWGQGTTVTVSS |

A-H.33

| | | |
|---|---|---|
| SEQ ID NO: | VH + VL | QVQLVQSGAEVKKPGSSVKVSCKASGTDFKLTYIHWVRQAP |
| 171 | | GQGLEWMGRISAGSGNTKYNEKFKGRVTITADTSTSTAYME |
| | | LSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVEDRVAWYQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVEDRVAWYQQKPG |
| 172 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 173 | | GQGLEWMGRISAGSGNTKYNEKFKGRVTITADTSTSTAYME |
| | | LSSLRSEDTAVYYCAGSYYSYDVLDYWGQGTTVTVSS |

A-H.34

| | | |
|---|---|---|
| SEQ ID NO: | VH + VL | QVQLVQSGAEVKKPGSSVKVSCKASGTDFRLTYIHWVRQAP |
| 174 | | GQGLEWMGRISPGSGNTKYNEKFKGRVTITADTSTSTAYME |
| | | LSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVGNRVAWYQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVGNRVAWYQQKPG |
| 175 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFRLTYIHWVRQAP |
| 176 | | GQGLEWMGRISPGSGNTKYNEKFKGRVTITADTSTSTAYME |
| | | LSSLRSEDTAVYYCAGSYYSYDVLDYWGQGTTVTVSS |

A-H.35

| | | |
|---|---|---|
| SEQ ID NO: | VH + VL | QVQLVQSGAEVKKPGSSVKVSCKASGHDFDKTYIHWVRQA |
| 177 | | PGQGLEWMGRVSAGSGNVKYNEKFKGRVTITADTSTSTAY |
| | | MELSSLRSEDTAVYYCAGSYYSYDVLDYWGQGTTVTVSSG |
| | | GGGSGGGGSGGGGSGGGGSDIQMTQSPSFLSASVGDRVTITC |
| | | KASQNVEDRVAWYQQKPGKAPKALIYSSSHRYKGVPSRFSG |
| | | SGSGTEFTLTISSLQPEDFATYFCQQFKSYPLTFGQGTKLEIK |
| SEQ ID NO: | VL | DIQMTQSPSELSASVGDRVTITCKASQNVEDRVAWYQQKPG |
| 178 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKTYIHWVRQA |
| 179 | | PGQGLEWMGRVSAGSGNVKYNEKFKGRVTITADTSTSTAY |
| | | MELSSLRSEDTAVYYCAGSYYSYDVLDYWGQGTTVTVSS |

A-H.36

| | | |
|---|---|---|
| SEQ ID NO: | VH + VL | QVQLVQSGAEVKKPGSSVKVSCKASGHDFKLTYIHWVRQA |
| 180 | | PGQGLEWMGRVSPGSGNTKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVGDRVTITCK |
| | | ASQNVEDRVAWHQQKPGKAPKALIYSSSHRYKGVPSRFSGS |
| | | GSGTEFTLTISSLQPEDFATYFCQQFKSYPLTFGQGTKLEIK |
| SEQ ID NO: | VL | DIQMTQSPSFLSASVGDRVTITCKASQNVEDRVAWHQQKPG |
| 181 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFKLTYIHWVRQA |
| 182 | | PGQGLEWMGRVSPGSGNTKYNEKFKGRVTITADTSTSTAYM |
| | | ELSSLRSEDTAVYYCAGSYYSYDVLDYWGQGTTVTVSS |

A-H.37

| | | |
|---|---|---|
| SEQ ID NO: | VH + VL | QVQLVQSGAEVKKPGSSVKVSCKASGHDFDKTYIHWVRQA |
| 183 | | PGQGLEWMGRIYPGSGNVKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVGDRVTITCK |
| | | ASQNVADRVAWYQQKPGKAPKALIYSSSHRYKGVPSRFSGS |
| | | GSGTEFTLTISSLQPEDFATYFCQQFKSYPLTFGQGTKLEIK |
| SEQ ID NO: | VL | DIQMTQSPSFLSASVGDRVTITCKASQNVADRVAWYQQKPG |
| 184 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKTYIHWVRQA |
| 185 | | PGQGLEWMGRIYPGSGNVKYNEKFKGRVTITADTSTSTAYM |
| | | ELSSLRSEDTAVYYCAGSYYSYDVLDYWGQGTTVTVSS |

A-H.38

| | | |
|---|---|---|
| SEQ ID NO: | VH + VL | QVQLVQSGAEVKKPGSSVKVSCKASGTDFDKTYIHWVRQA |
| 186 | | PGQGLEWMGRISAGSGNVKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVQDRVTITQK |
| | | ASQNVDDRVAWYQQKPGKAPKALIYSSSHRYKGVPSRFSGS |
| | | GSGTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 187 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDEDKTYIHWVRQA |
| 188 | | PGQGLEWMGRISAGSGNVKYNEKFKGRVTITADTSTSTAYM |
| | | ELSSLRSEDTAVYYCAGSYYSYDVLDYWGQGTTVTVSS |

A-H.39

| | | |
|---|---|---|
| SEQ ID NO: | VH + VL | QVQLVQSGAEVKKPGSSVKVSCKASGTDFDKIYIHWVRQAP |
| 189 | | GQGLEWMGRISAGSGNIKYNEKFKGRVTITADTSTSTAYME |
| | | LSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVDDRVAWYQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 190 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 191 | | GQGLEWMGRISAGSGNIKYNEKFKGRVTITADTSTSTAYME |
| | | LSSLRSEDTAVYYCAGSYYSYDVLDYWGQGTTVTVSS |

A-H.40

| | | |
|---|---|---|
| SEQ ID NO: | VH + VL | QVQLVQSGAEVKKPGSSVKVSCKASGTDFDKIYIHWVRQAP |
| 192 | | GQGLEWMGRISAGSGNVKYNEKFKGRVTITADTSTSTAYME |
| | | LSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVGDRVAWYQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 193 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 194 | | GQGLEWMGRISAGSGNVKYNEKFKGRVTITADTSTSTAYME |
| | | LSSLRSEDTAVYYCAGSYYSYDVLDYWGQGTTVTVSS |

A-H.41

| | | |
|---|---|---|
| SEQ ID NO: | VH + VL | QVQLVQSGAEVKKPGSSVKVSCKASGGTFKLTYIHWVRQAP |
| 195 | | GQGLEWMGRVSAGSGNVKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVGDRVTITCK |
| | | ASQNVDDRVAWYQQKPGKAPKALIYSSSHRYKGVPSRFSGS |
| | | GSGTEFTLTISSLQPEDFATYFCQQFKSYPLTFGQGTKLEIK |
| SEQ ID NO: | VL | DIQMTQSPSELSASVGDRVTITCKASQNVDDRVAWYQQKPG |
| 196 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFKLTYIHWVRQAP |
| 197 | | GQGLEWMGRVSAGSGNVKYNEKFKGRVTITADTSTSTAYM |
| | | ELSSLRSEDTAVYYCAGSYYSYDVLDYWGQGTTVTVSS |

A-H.42

| | | |
|---|---|---|
| SEQ ID NO: | VH + VL | QVQLVQSGAEVKKPGSSVKVSCKASGTDFKLTYIHWVRQAP |
| 198 | | GQGLEWMGRISPGSGNVKYNEKFKGRVTITADTSTSTAYME |
| | | LSSLRSEDTAVYYCAGSYYSYDVLDYWGQGTTVTVSSGGQ |
| | | GSGGGGSGGGGSGGGGSDIQMTQSPSFLSASVGDRVTITCKA |
| | | SQNVDNRVAWHQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVDNRVAWHQQKPG |
| 199 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 200 | | GQGLEWMGRISPGSGNVKYNEKFKGRVTITADTSTSTAYME |
| | | LSSLRSEDTAVYYCAGSYYSYDVLDYWGQGTTVTVSS |

A-H.43

| | | |
|---|---|---|
| SEQ ID NO: | VH + VL | QVQLVQSGAEVKKPGSSVKVSCKASGHDFDKFYIHWVRQA |
| 201 | | PGQGLEWMGRVSAGSGNTKYNEKFKGRVTITADTSTSTAY |
| | | MELSSLRSEDTAVYYCAGSYYSYDVLDYWGQGTTVTVSSG |
| | | GGGSGGGGSGGGGSGGGGSDIQMTQSPSFLSASVGDRVTITC |
| | | KASQNVDNRVAWYQQKPGKAPKALIYSSSHRYKGVPSRFSG |
| | | SGSGTEFTLTISSLQPEDFATYFCQQFKSYPLTFGQGTKLEIK |
| SEQ ID NO: | VL | DIQMTQSPSFLSASVGDRVTITCKASQNVDNRVAWYQQKPG |
| 202 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQA |
| 203 | | PGQGLEWMGRVSAGSGNTKYNEKFKGRVTITADTSTSTAY |
| | | MELSSLRSEDTAVYYCAGSYYSYDVLDYWGQGTTVTVSS |

A-H.44

| | | |
|---|---|---|
| SEQ ID NO: | VH + VL | QVQLVQSGAEVKKPGSSVKVSCKASGTDFDKFYIHWVRQAP |
| 204 | | GQGLEWMGRVSAGSGNVKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVGDRVTITCK |
| | | ASQNVGDRVVWYQQKPGKAPKALIYSSSHRYKGVPSRFSGS |
| | | GSGTEFTLTISSLQPEDFATYFCQQFKSYPLTFGQGTKLEIK |
| SEQ ID NO: | VH | QVQLVQSGAEVKKPGSSVKVSCKASGTDFDKFYIHWVRQAP |
| 205 | | GQGLEWMGRVSAGSGNVKYNEKFKGRVTITADTSTSTAYM |
| | | ELSSLRSEDTAVYYCAGSYYSYDVLDYWGQGTTVTVSS |

A-H.45

| | | |
|---|---|---|
| SEQ ID NO: | VH + VL | QVQLVQSGAEVKKPGSSVKVSCKASGYSFTTYYIHWVRQAP |
| 206 | | GQGLEWMGWFSAGSGNTKYNEKFKGRVTITADTSTSTAYM |
| | | ELSSLRSEDTAVYYCAVSYYSYDVLDYWGQGTTVTVSSGG |
| | | GGSGGGGSGGGGSGGGGSDIQMTQSPSFLSASVGDRVTITCK |
| | | ASQNVGINVVWHQQKPGKAPKALIYSSSHRYSGVPSRFSGS |
| | | GSGTEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 207 | | GQGLEWMGWFSAGSGNTKYNEKFKGRVTITADTSTSTAYM |
| | | ELSSLRSEDTAVYYCAVSYYSYDVLDYWGQGTTVTVSS |

A-H.46

| | | |
|---|---|---|
| SEQ ID NO: | VH + VL | QVQLVQSGAEVKKPGSSVKVSCKASGYSFTTYYIHWVRQAP |
| 208 | | GQGLEWMGWFSAGSGNTKYNEKFKQRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVGDRVTITCK |
| | | ASQNVGINVVWHQQKPGKAPKALIYSSSHRYSGVPSRFSGS |
| | | GSGTEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 209 | | GQGLEWMGWFSAGSGNTKYNEKFKQRVTITADTSTSTAYM |
| | | ELSSLRSEDTAVYYCAGSYYSYDVLDYWGQGTTVTVSS |

A-H.47

| | | |
|---|---|---|
| SEQ ID NO: | VH + VL | QVQLVQSGAEVKKPGSSVKVSCKASGYSFTTYYIHWVRQAP |
| 210 | | GQGLEWMGWFFPGSGNTKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVGDRVTITCK |
| | | ASQNVGINVVWHQQKPGKAPKALIYSSSHRYSGVPSRFSGS |
| | | GSGTEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 211 | | GQGLEWMGWFFPGSGNTKYNEKFKGRVTITADTSTSTAYM |
| | | ELSSLRSEDTAVYYCAGSYYSYDVLDYWGQGTTVTVSS |

A-H.48

| | | |
|---|---|---|
| SEQ ID NO: | VH + VL | QVQLVQSGAEVKKPGSSVKVSCKASGYSFTTYYIHWVRQAP |
| 212 | | GQGLEWMGWFSPGSGNTKYNEKFKGRVTITADTSTSTAYM |
| | | ELSSLRSEDTAVYYCAVSYYSYDVLDYWGQGTTVTVSSGG |
| | | GGSGGGGSGGGGSGGGGSDIQMTQSPSFLSASVGDRVTITCK |
| | | ASQNVGINVVWHQQKPGKAPKALIYSSSHRYSGVPSRFSGS |
| | | GSGTEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 213 | | GQGLEWMGWFSPGSGNTKYNEKFKGRVTITADTSTSTAYM |
| | | ELSSLRSEDTAVYYCAVSYYSYDVLDYWGQGTTVTVSS |

A-H.49

| | | |
|---|---|---|
| SEQ ID NO: | VH + VL | QVQLVQSGAEVKKPGSSVKVSCKASGYSFTTYYIHWVRQAP |
| 214 | | GQGLEWMGWFSPGSGNTKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVGDRVTITCK |
| | | ASQNVGINVVWHQQKPGKAPKALIYSSSHRYSGVPSRFSGS |
| | | GSGTEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 215 | | GQGLEWMGWFSPGSGNTKYNEKFKGRVTITADTSTSTAYM. |
| | | ELSSLRSEDTAVYYCAGSYYSYDVLDYWGQGTTVTVSS |

A-H.50

| | | |
|---|---|---|
| SEQ ID NO: | VH + VL | QVQLVQSGAEVKKPGSSVKVSCKASGYSFTTYYIHWVRQAP |
| 216 | | GQGLEWMGRIFPQSGNIKYNEKFKGRVTITADTSTSTAYMEL |
| | | SSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGS |
| | | GTEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 217 | | GQGLEWMGRIFPQSGNIKYNEKFKGRVTITADTSTSTAYMEL |
| | | SSLRSEDTAVYYCAGSYYSYDVLDYWGQGTTVTVSS |

A-H.51

| | | |
|---|---|---|
| SEQ ID NO: | VH + VL | QVQLVQSGAEVKKPGSSVKVSCKASGYSFTTYYIHWVRQAP |
| 218 | | GQGLEWMGWFFPGSGNIKYNEKFKGRVTITADTSTSTAYME |
| | | LSSLRSEDTAVYYCAGSIYSAGVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGS |
| | | GTEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 219 | | GQGLEWMGWFFPGSGNIKYNEKFKGRVTITADTSTSTAYME |
| | | LSSLRSEDTAVYYCAGSIYSAGVLDYWGQGTTVTVSS |

A-H.52

| | | |
|---|---|---|
| SEQ ID NO: | VH + VL | QVQLVQSGAEVKKPGSSVKVSCKASGYSFTLGYIHWVRQAP |
| 220 | | GQGLEWMGWFFPGSGNIKYNEKFKGRVTITADTSTSTAYME |
| | | LSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVGINVVWHQQKPGKAPKALIYSSSHRYSGVPSRFSGSGS |
| | | GTEFTLTISSLQPEDFATYFCQQFKSYPLTFGQGTKLEIK |
| SEQ ID NO: | VH | QVQLVQSGAEVKKPGSSVKVSCKASGYSFTLGYIHWVRQAP |
| 221 | | GQGLEWMGWFFPGSGNIKYNEKFKGRVTITADTSTSTAYME |
| | | LSSLRSEDTAVYYCAGSYYSYDVLDYWGQGTTVTVSS |

A-H.53

| | | |
|---|---|---|
| SEQ ID NO: | VH + VL | QVQLVQSGAEVKKPGSSVKVSCKASGYSFRLTYIHWVRQAP |
| 222 | | GQGLEWMGWFFPGSGNIKYNEKFKGRVTITADTSTSTAYME |
| | | LSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVGINVVWHQQKPGKAPKALIYSSSHRYSGVPSRFSGSGS |
| | | GTEFTLTISSLQPEDFATYFCQQFKSYPLTFGQGTKLEIK |
| SEQ ID NO: | VH | QVQLVQSGAEVKKPGSSVKVSCKASGYSFRLTYIHWVRQAP |
| 223 | | GQGLEWMGWFFPGSGNIKYNEKFKGRVTITADTSTSTAYME |
| | | LSSLRSEDTAVYYCAGSYYSYDVLDYWGQGTTVTVSS |

A-H.54

| | | |
|---|---|---|
| SEQ ID NO: | VH + VL | QVQLVQSGAEVKKPGSSVKVSCKASGYSFHNWYIHWVRQA |
| 224 | | PGQGLEWMGWFFPGSGNIKYNEKFKGRVTITADTSTSTAYM |
| | | ELSSLRSEDTAVYYCAGSYYSYDVLDYWGQGTTVTVSSGG |
| | | GGSGGGGSGGGGSGGGGSDIQMTQSPSFLSASVGDRVTITCK |
| | | ASQNVGINVVWHQQKPGKAPKALIYSSSHRYSGVPSRFSGS |
| | | GSGTEFTLTISSLQPEDFATYFCQQFKSYPLTFGQGTKLEIK |
| SEQ ID NO: | VH | QVQLVQSGAEVKKPGSSVKVSCKASGYSFHNWYIHWVRQA |
| 225 | | PGQGLEWMGWFFPGSGNIKYNEKFKGRVTITADTSTSTAYM |
| | | ELSSLRSEDTAVYYCAGSYYSYDVLDYWGQGTTVTVSS |

A-H.55 antibody

| | | |
|---|---|---|
| SEQ ID NO: 3 | HC CDR1 (Combined) | GYSFTTYYIH |
| SEQ ID NO: 4 | HC CDR2 (Combined) | WFFPGSGNIK YNEKFKG |
| SEQ ID NO: 5 | HC CDR3 (Combined) | SYYSYDVLDY |
| SEQ ID NO: 45 | HC CDR1 (Kabat) | TYYIH |
| SEQ ID NO: 46 | HC CDR2 (Kabat) | WFFPGSGNIKYNEKFKG |
| SEQ ID NO: 47 | HC CDR3 (Kabat) | SYYSYDVLDY |
| SEQ ID NO: 48 | HC CDR1 (Chothia) | GYSFTTY |
| SEQ ID NO: 49 | HC CDR2 (Chothia) | FPGSGN |
| SEQ ID NO: 50 | HC CDR3 (Chothia) | SYYSYDVLDY |
| SEQ ID NO: | VH | QVQLVQSGAEVKKPGSSVKVSCKASGYSFTTYYIHWVRQAP |
| 1100 | | GQGLEWMGWFFPGSGNIKYNEKFKGRVTITADTSTSTAYME |
| | | LSSLRSEDTAVYYCAGSYYSYDVLDYWGQGTTVTVSS |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 51 | LC CDR1 (Kabat) | KASQNVGINVV |
| SEQ ID NO: 52 | LC CDR2 (Kabat) | SSSHRYS |
| SEQ ID NO: 53 | LC CDR3 (Kabat) | QQFKSYPLT |
| SEQ ID NO: 54 | LC CDR1 (Chothia) | KASQNVGINVV |
| SEQ ID NO: 55 | LC CDR2 (chothia) | SSSHRYS |
| SEQ ID NO: 56 | LC CDR3 (chothia) | QQFKSYPLT |
| SEQ ID NO: | VL | QSVLTQPPSVSEAPRQRVTISCKASQNVGINVVWHQQLPGK |
| 1101 | | APKALIYSSSHRYSGVSDRFSGSGSGTSFSLAISGLQSEDEAD |
| | | YFCQQFKSYPLTFGTGTKVTVL |

A-H.56

| | | |
|---|---|---|
| SEQ ID NO: | VH+ VL (ScFv) | QVQLVQSGAEVKKPGSSVKVSCKASGHDEDKFYIHWVRQA |
| 1309 | | PGQGLEWMGRVSAGSGNVKYNEKFKGRVTITADTSTSTAY |
| | | MELSSLRSEDTAVYYCAGSYYSYDVLDYWGQGTTVTVSSG |
| | | GGGSGGGGSGGGGSGGGGSDIQMTQSPSFLSASVGDRVTITC |
| | | KASQNVGNRVAWYQQKPGKAPKALIYSSSHRYKGVPSRFSG |
| | | SGSGTEFTLTISSLQPEDFATYFCQQFKSYPLTFGQGTKLEIK |

A-H.57

| | | |
|---|---|---|
| SEQ ID NO: | VH+ VL (ScFv) | QVQLVQSGAEVKKPGSSVKVSCKASGHDFRLTYIHWVRQAP |
| 1326 | | GQGLEWMGRVSAGSQNTKYNEKFKGRVTITADTSTSTAYM |
| | | ELSSLRSEDTAVYYCAVSYYSYDVLDYWGQGTTVTVSSGG |
| | | GGSGGGGSGGGGSGGGGSDIQMTQSPSFLSASVGDRVTITCK |
| | | ASQNVGDRVVWHQQKPGKAPKALIYSSSHRYKGVPSRFSGS |
| | | GSGTEFTLTISSLQPEDFATYFCQQFKSYPLTFGQGTKLEIK |

A-H.58

| | | |
|---|---|---|
| SEQ ID NO: | VH+ VL (ScFv) | QVQLVQSGAEVKKPGSSVKVSCKASGHDFRLTYIHWVRQAP |
| 1327 | | GQGLEWMGRVSAGSGNVKYNEKFKQRVTITADTSTSTAYM |
| | | ELSSLRSEDTAVYYCAVSYYSYDVLDYWGQGTTVTVSSGG |
| | | GGSGGGGSGGGGSGGGGSDIQMTQSPSFLSASVGDRVTITCK |
| | | ASQNVGNRVVWHQQKPGKAPKALIYSSSHRYKGVPSRFSGS |
| | | GSGTEFTLTISSLQPEDFATYFCQQFKSYPLTFGQGTKLEIK |

A-H.59

| | | |
|---|---|---|
| SEQ ID NO: | VH+ VL (ScFv) | QVQLVQSGAEVKKPGSSVKVSCKASGHDFRLTYIHWVRQAP |
| 1328 | | GQGLEWMGRIYAGSGNVKYNEKFKGRVTITADTSTSTAYM |
| | | ELSSLRSEDTAVYYCAVSYYSYDVLDYWGQGTTVTVSSGG |
| | | GGSGGGGSGGGGSGGGGSDIQMTQSPSFLSASVGDRVTITCK |
| | | ASQNVADRVVWHQQKPGKAPKALIYSSSHRYKGVPSRFSGS |
| | | GSGTEFTLTISSLQPEDFATYFCQQFKSYPLTFGQGTKLEIK |

A-H.60

| | | |
|---|---|---|
| SEQ ID NO: | VH+ VL (ScFv) | QVQLVQSGAEVKKPGSSVKVSCKASGHDFKLTYIHWVRQA |
| 1329 | | PGQGLEWMGRVSAGSGNTKYNEKFKGRVTITADTSTSTAY |
| | | MELSSLRSEDTAVYYCAVSYYSYDVLDYWGQGTTVTVSSG |
| | | GGGSGGGGSGGGGGGGGSDIQMTQSPSFLSASVGDRVTITC |
| | | KASQNVGDRVAWHQQKPGKAPKALIYSSSHRYKGVPSRFSG |
| | | SGSGTEFTLTISSLQPEDFATYFCQQFKSYPLTFGQGTKLEIK |

A-H.61

| | | |
|---|---|---|
| SEQ ID NO: | VH+ VL (ScFv) | QVQLVQSGAEVKKPGSSVKVSCKASGHDFKLTYIHWVRQA |
| 1330 | | PGQGLEWMGRVSAGSGNTKYNEKFKGRVTITADTSTSTAY |
| | | MELSSLRSEDTAVYYCAVSYYSYDVLDYWGQGTTVTVSSG |
| | | GGGSGGGGSGGGGSGGGGSDIQMTQSPSFLSASVGDRVTITC |
| | | KASQNVDNRVAWHQQKPGKAPKALIYSSSHRYKGVPSRFSG |
| | | SGSGTEFTLTISSLQPEDFATYFCQQFKSYPLTFGQGTKLEIK |

A-H.62

| | | |
|---|---|---|
| SEQ ID NO: | VH+ VL (ScFv) | QVQLVQSGAEVKKPGSSVKVSCKASGHDFRLTYIHWVRQAP |
| 1331 | | GQGLEWMGRVSAGSGNVKYNEKFKGRVTITADTSTSTAYM |
| | | ELSSLRSEDTAVYYCAVSYYSYDVLDYWGQGTTVTVSSGG |
| | | GGSGGGGGGGGSGGGGSDIQMTQSPSFLSASVGDRVTITCK |
| | | ASQNVADRVVWHQQKPGKAPKALIYSSSHRYKGVPSRFSGS |
| | | GSGTEFTLTISSLQPEDFATYFCQQFKSYPLTFGQGTKLEIK |

A-H.63

| | | |
|---|---|---|
| SEQ ID NO: | VH+ VL (ScFv) | QVQLVQSGAEVKKPGSSVKVSCKASGHDFRLTYIHWVRQAP |
| 1332 | | GQGLEWMGRVYAGSGNTKYNEKFKGRVTITADTSTSTAYM |
| | | ELSSLRSEDTAVYYCAVSYYSYDVLDYWGQGTTVTVSSGG |
| | | GGSGGGGSGGGGSGGGGSDIQMTQSPSFLSASVGDRVTITCK |
| | | ASQNVEDRVVWHQQKPGKAPKALIYSSSHRYKGVPSRFSGS |
| | | GSGTEFTLTISSLQPEDFATYFCQQFKSYPLTFGQGTKLEIK |

A-H.64

| | | |
|---|---|---|
| SEQ ID NO: | VH+ VL (SeFv) | QVQLVQSGAEVKKPGSSVKVSCKASGHDFKLTYIHWVRQA |
| 1333 | | PGQGLEWMGRVSAGSGNTKYNEKFKGRVTITADTSTSTAY |
| | | MELSSLRSEDTAVYYCAVSYYSYDVLDYWGQGTTVTVSSG |
| | | GGGSGGGGSGGGGSGGGGSDIQMTQSPSFLSASVGDRVTITC |
| | | KASQNVADRVVWHQQKPGKAPKALIYSSSHRYKGVPSRFSG |
| | | SGSGTEFTLTISSLQPEDFATYFCQQFKSYPLTFGQGTKLEIK |

A-H.65

| | | |
|---|---|---|
| SEQ ID NO: | VH+ VL (ScFv) | QVQLVQSGAEVKKPGSSVKVSCKASGHDFKLTYIHWVRQA |
| 1334 | | PGQGLEWMGRISAGSGNTKYNEKFKGRVTITADTSTSTAYM |
| | | ELSSLRSEDTAVYYCAVSYYSYDVLDYWGQGTTVTVSSGG |
| | | GGSGGGGSGGGGSGGGGSDIQMTQSPSFLSASVGDRVTITCK |
| | | ASQNVGDRVVWHQQKPGKAPKALIYSSSHRYKGVPSRESGS |
| | | GSGTEFTLTISSLQPEDFATYFCQQFKSYPLTFGQGTKLEIK |

A-H.66

| | | |
|---|---|---|
| SEQ ID NO: | VH+ VL (ScFv) | QVQLVQSGAEVKKPGSSVKVSCKASGHDFKLTYIHWVRQA |
| 1335 | | PGQGLEWMGRIYAGSGNTKYNEKFKGRVTITADTSTSTAYM |
| | | ELSSLRSEDTAVYYCAVSYYSYDVLDYWGQGTTVTVSSGG |
| | | GGSGGGGSGGGGSGGGGSDIQMTQSPSFLSASVGDRVTITCK |
| | | ASQNVGDRVVWHQQKPGKAPKALIYSSSHRYKGVPSRFSGS |
| | | GSGTEFTLTISSLQPEDFATYFCQQFKSYPLTFGQGTKLEIK |

A-H.67

| | | |
|---|---|---|
| SEQ ID NO: | VH+ VL (ScFv) | QVQLVQSGAEVKKPGSSVKVSCKASGTDFKLTYIHWVRQAP |
| 1336 | | GQGLEWMGRIFPGSGNVKYNEKFKGRVTITADTSTSTAYME |
| | | LSSLRSEDTAVYYCAVSYYSYDVLDYWGQGTTVTVSSGGG |
| | | GSGGGGSGGGGSGGGGSDIQMTQSPSFLSASVGDRVTITCKA |
| | | SQNVDNRVAWYQQKPGKAPKALIYSSSHRYKGVPSRFSGSG |
| | | SGTEFTLTISSLQPEDFATYFCQQFKSYPLTFGQGTKLEIK |

A-H.68

| | | |
|---|---|---|
| SEQ ID NO: | VH+ VL (ScFv) | QVQLVQSGAEVKKPGSSVKVSCKASGHDFRLTYIHWVRQAP |
| 1337 | | GQGLEWMGRISAGSGNVKYNEKFKGRVTITADTSTSTAYME |
| | | LSSLRSEDTAVYYCAVSYYSYDVLDYWGQGTTVTVSSGGG |
| | | GSGGGGSGGGGSGGGGSDIQMTQSPSFLSASVGDRVTITCKA |
| | | SQNVADRVAWYQQKPGKAPKALIYSSSHRYKGVPSRFSGSG |
| | | SGTEFTLTISSLQPEDFATYFCQQFKSYPLTFGQGTKLEIK |

A-H.69 (also referred to as A-H.13)

| | | |
|---|---|---|
| SEQ ID NO: | VH+ VL (ScFv) | QVQLVQSGAEVKKPGSSVKVSCKASGTDFKLTYIHWVRQAP |
| 110 | | GQGLEWMGRIFPGSGNVKYNEKFKGRVTITADTSTSTAYME |
| | | LSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVDNRVAWYQQKPGKAPKALIYSSSHRYKGVPSRFSGSG |
| | | SGTEFTLTISSLQPEDFATYFCQQFKSYPLTFGQGTKLEIK |

A-H humanized-matured VH

| | | |
|---|---|---|
| SEQ ID NO: | VH-humanized | QVQLVQSGAEVKKPGSSVKVSCKASGTDFKLTYIHWVRQAP |
| 1310 | matured 1 | GQGLEWMGRIFPGSGNVKYNEKFKGRVTITADTSTSTAYME |
| | | LSSLRSEDTAVYYCAGSYYSYDVLDYWGQGTTVTVSS |
| SEQ ID NO: | VH-humanized | QVQLVQSGAEVKKPGSSVKVSCKASGTDFKLTYIHWVRQAP |
| 1311 | matured 2 | GQGLEWMGRIFPGSGNVKYNEKFKGRVTITADTSTSTAYME |
| | | LSSLRSEDTAVYYCAVSYYSYDVLDYWGQGTTVTVSS |
| SEQ ID NO: | VH-humanized | QVQLVQSGAEVKKPGSSVKVSCKASGHDFRLTYIHWVRQAP |
| 1312 | matured 3 | GQGLEWMGRISAGSGNVKYNEKFKGRVTITADTSTSTAYME |
| | | LSSLRSEDTAVYYCAVSYYSYDVLDYWGQGTTVTVSS |

A-H humanized-matured VL

| | | |
|---|---|---|
| SEQ ID NO: | VL-humanized | DIQMTQSPSFLSASVGDRVTITCKASQNVDNRVAWYQQKPG |
| 1313 | matured 1 | KAPKALIYSSSHRYKGVPSRFSGSGSGTEFTLTISSLQPEDFAT |
| | | YFCQQFKSYPLTFGQGTKLEIK |
| SEQ ID NO: | VL-humanized | DIQMTQSPSFLSASVGDRVTITCKASQNVADRVAWYQQKPG |
| 1314 | matured 2 | KAPKALIYSSSHRYKGVPSRFSGSGSGTEFTLTISSLQPEDFAT |
| | | YFCQQFKSYPLTFGQGTKLEIK |

A-H.70

| | | |
|---|---|---|
| SEQ ID NO: | HC CDR1 | GHDFRLTYIH |
| 3650 | | |
| SEQ ID NO: | HC CDR2 | RVSAGSGNVKYNEKFKG |
| 3651 | | |
| SEQ ID NO: 5 | HC CDR3 | SYYSYDVLDY |
| SEQ ID NO: | LC CDR1 | KASQNVGNRVV |
| 652 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1346 | (CDRs underlined) | GQGLEWMGRVSAGSQNVKYNEKFKGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVGNRVVWHQQKPG |
| 1347 | (CDRs underlined) | KAPKALIYSSSHRYKGVPSRFSGSGSGTEFTLTISSLQPEDFAT |
| | | YFCQQFKSYPLTFGQGTKLEIK |

A-H.71

| | | |
|---|---|---|
| SEQ ID NO: | HC CDR1 | GHDFRLTYIH |
| 3650 | | |
| SEQ ID NO: | HC CDR2 | RIYAGSGNVKYNEKFKG |
| 3654 | | |
| SEQ ID NO: 5 | HC CDR3 | SYYSYDVLDY |
| SEQ ID NO: | LC CDR1 | KASQNVADRVV |
| 3655 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1348 | (CDRs underlined) | GQGLEWMGRIYAGSGNVKYNEKFKGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVADRVVWHQQKPG |
| 1349 | (CDRs underlined) | KAPKALIYSSSHRYKGVPSRFSGSGSGTEFTLTISSLQPEDFAT |
| | | YFCQQEKSYPLTFGQGTKLEIK |

A-H.72

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVGDRVA |
| 3658 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQA |
| 1350 | (CDRs underlined) | PGQGLEWMGRVSAGSGNTKYNEKFKGRVTITADTSTSTAY |
| | | MELSSLRSEDTAVYYCAVSYYSYDVLDYWGQGTTVTVSS |
| SEQ ID NO: | VL | DIQMTQSPSFLSASVGDRVTITCKASQNVGDRVAWHQQKPG |
| 1351 | (CDRs underlined) | KAPKALIYSSSHRYKGVPSRFSGSGSGTEFTLTISSLQPEDFAT |
| | | YFCQQFKSYPLTFGQGTKLEIK |

A-H.73

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQA |
| 1350 | (CDRs underlined) | PGQGLEWMGRVSAGSGNTKYNEKFKGRVTITADTSTSTAY |
| | | MELSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1353 | (CDRs underlined) | KAPKALIYSSSHRYKGVPSRFSGSGSGTEFTLTISSLQPEDFAT |
| | | YFCQQEKSYPLTFGQGTKLEIK |

A-H.74 (BKM0259)

| | | |
|---|---|---|
| SEQ ID NO: | HC CDR1 | GHDFRLTYIH |
| 3650 | | |
| SEQ ID NO: | HC CDR2 | RVSAGSGNVKYNEKFKG |
| 3651 | | |
| SEQ ID NO: 5 | HC CDR3 | SYYSYDVLDY |
| SEQ ID NO: | LC CDR1 | KASQNVADRVV |
| 3655 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1346 | (CDRs underlined) | GQGLEWMGRVSAGSGNVKYNEKFKGRVTITADISTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVADRVVWHQQKPG |
| 1349 | (CDRs underlined) | KAPKALIYSSSHRYKGVPSRFSGSGSGTEFTLTISSLQPEDFAT |
| | | YFCQQFKSYPLTFGQGTKLEIK |

A-H.75

| | | |
|---|---|---|
| SEQ ID NO: | HC CDR1 | GHDFRLTYIH |
| 3650 | | |
| SEQ ID NO: | HC CDR2 | RVYAGSGNTKYNEKFKG |
| 3659 | | |
| SEQ ID NO: 5 | HC CDR3 | SYYSYDVLDY |
| SEQ ID NO: | LC CDR1 | KASQNVEDRVV |
| 3660 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1356 | (CDRs underlined) | GQGLEWMGRVYAGSGNTKYNEKFKGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVEDRVVWHQQKPG |
| 1357 | (CDRs underlined) | KAPKALIYSSSHRYKGVPSRFSGSGSGTEFTLTISSLQPEDFAT |
| | | YFCQQFKSYPLTFGQGTKLEIK |

A-H.76

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVADRVV |
| 3655 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQA |
| 1350 | (CDRs underlined) | PGQGLEWMGRVSAGSGNTKYNEKFKGRVTITADTSTSTAY |
| | | MELSSLRSEDTAVYYCAVSYYSYDVLDYWGQGTTVTVSS |
| SEQ ID NO: | VL | DIQMTQSPSFLSASVGDRVTITCKASQNVADRVVWHQQKPG |
| 1349 | (CDRs underlined) | KAPKALIYSSSHRYKGVPSRFSGSGSGTEFTLTISSLQPEDFAT |
| | | YFCQQFKSYPLTFGQGTKLEIK |

A-H.77

| | | |
|---|---|---|
| SEQ ID NO: | HC CDR1 | GHDFKLTYIH |
| 3656 | | |
| SEQ ID NO: | HC CDR2 | RISAGSGNTKYNEKFKG |
| 3661 | | |
| SEQ ID NO: 5 | HC CDR3 | SYYSYDVLDY |
| SEQ ID NO: | LC CDR1 | KASQNVGDRVV |
| 3662 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQA |
| 1360 | (CDRs underlined) | PGQGLEWMGRISAGSGNTKYNEKFKGRVTITADTSTSTAYM |
| | | ELSSLRSEDTAVYYCAVSYYSYDVLDYWGQGTTVTVSS |
| SEQ ID NO: | VL | DIQMTQSPSFLSASVQDRVTITCKASQNVGDRVVWHQQKPG |
| 1361 | (CDRs underlined) | KAPKALIYSSSHRYKGVPSRFSGSGSGTEFTLTISSLQPEDFAT |
| | | YFCQQFKSYPLTFGQGTKLEIK |

A-H.78

| | | |
|---|---|---|
| SEQ ID NO: | HC CDR1 | GHDFKLTYIH |
| 3656 | | |
| SEQ ID NO: | HC CDR2 | RIYAGSGNTKYNEKFKG |
| 3663 | | |
| SEQ ID NO: 5 | HC CDR3 | SYYSYDVLDY |
| SEQ ID NO: | LC CDR1 | KASQNVGDRVV |
| 3662 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQA |
| 1362 | (CDRs underlined) | PGQGLEWMGRIYAGSGNTKYNEKFKGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVGDRVVWHQQKPG |
| 1361 | (CDRs underlined) | KAPKALIYSSSHRYKGVPSRFSGSGSGTEFTLTISSLQPEDFAT |
| | | YFCQQFKSYPLTFGQGTKLEIK |

A-H.79

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | RASQNVDNRLG |
| 3666 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQA |
| 1350 | (CDRs underlined) | PGQGLEWMGRVSAGSGNTKYNEKFKGRVTITADTSTSTAY |
| | | MELSSLRSEDTAVYYCAVSYYSYDVLDYWGQGTTVTVSS |
| SEQ ID NO: | VL | DIQMTQSPSFLSASVGDRVTITCRASQNVDNRLGWHQQKPG |
| 1365 | (CDRs underlined) | KAPKALIYSSSHRYKGVPSRFSGSGSGTEFTLTISSLQPEDFAT |
| | | YFCQQFKSYPLTFGQGTKLEIK |

A-H.80

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | AASSLQK |
| 3668 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQA |
| 1350 | (CDRs underlined) | PGQGLEWMGRVSAGSGNTKYNEKFKGRVTITADTSTSTAY |
| | | MELSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1367 | (CDRs underlined) | KAPKALIYAASSLQKGVPSRFSGSGSGTEFTLTISSLQPEDFAT |
| | | YFCQQEKSYPLTFGQGTKLEIK |

A-H.81

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | SSSHRYK |
| 3653 | | |
| SEQ ID NO: | LC CDR3 | LQHNSYPLT |
| 3664 | | |
| SEQ ID NO: | VH | QVQLVQSGAEVKKPGSSVKVSCKASGHDFKLTYIHWVRQA |
| 1350 | (CDRs underlined) | PGQGLEWMGRVSAQSGNTKYNEKFKGRVTITADTSTSTAY |
| | | MELSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1369 | (CDRs underlined) | KAPKALIYSSSHRYKGVPSRFSGSGSGTEFTLTISSLQPEDFAT |
| | | YFCLQHNSYPLTFGQGTKLEIK |

A-H.82

| | | |
|---|---|---|
| SEQ ID NO: | HC CDR1 | GHDFKLTYIH |
| 3656 | | |
| SEQ ID NO: | HC CDR2 | RVSAGSGNVNYAQKFQG |
| 3665 | | |
| SEQ ID NO: 5 | HC CDR3 | SYYSYDVLDY |
| SEQ ID NO: | LC CDR1 | RASQNVDNRLG |
| 3666 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQA |
| 1370 | (CDRs underlined) | PGQGLEWMGRVSAGSGNVNYAQKFQGRVTITADTSTSTAY |
| | | MELSSLRSEDTAVYYCAVSYYSYDVLDYWGQGTTVTVSS |
| SEQ ID NO: | VL | DIQMTQSPSFLSASVGDRVTITCRASQNVDNRLGWHQQKPG |
| 1365 | (CDRs underlined) | KAPKALIYSSSHRYKGVPSRFSGSGSGTEFTLTISSLQPEDFAT |
| | | YFCQQFKSYPLTFGQGTKLEIK |

A-H.83

| | | |
|---|---|---|
| SEQ ID NO: | HC CDR1 | GHDFKLTYIH |
| 3656 | | |
| SEQ ID NO: | HC CDR2 | RVSAGSGNVNYAQKFQG |
| 3665 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | AASSLQK |
| 3668 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQA |
| 1370 | (CDRs underlined) | PGQGLEWMGRVSAGSGNVNYAQKFQGRVTITADTSTSTAY |
| | | MELSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1367 | (CDRs underlined) | KAPKALIYAASSLQKGVPSRFSGSGSGTEFTLTISSLQPEDFAT |
| | | YFCQQFKSYPLTFGQGTKLEIK |

A-H.84

| | | |
|---|---|---|
| SEQ ID NO: | HC CDR1 | GHDFKLTYIH |
| 3656 | | |
| SEQ ID NO: | HC CDR2 | RVSAGSGNVNYAQKFQG |
| 3665 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | SSSHRYK |
| 3653 | | |
| SEQ ID NO: | LC CDR3 | LQHNSYPLT |
| 3664 | | |
| SEQ ID NO: | VH | QVQLVQSGAEVKKPGSSVKVSCKASGHDFKLTYIHWVRQA |
| 1370 | (CDRs underlined) | PGQGLEWMGRVSAGSGNVNYAQKFQGRVTITADTSTSTAY |
| | | MELSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1369 | (CDRs underlined) | KAPKALIYSSSHRYKGVPSRFSGSGSGTEFTLTISSLQPEDFAT |
| | | YFCLQHNSYPLTFGQGTKLEIK |

A-H.85

| | | |
|---|---|---|
| SEQ ID NO: | HC CDR1 | GHDFRLTYIH |
| 3650 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVGDRVV |
| 3662 | | |
| SEQ ID NO: | LC CDR2 | SSSHRYK |
| 3653 | | |
| SEQ ID NO: 8 | LC CDR3 | QQFKSYPLT |
| SEQ ID NO: | VH (CDRs underlined) | QVQLVQSGAEVKKPGSSVKVSCKASGHDFRLTYIHWVRQAP |
| 1344 | | GQGLEWMGRVSAGSGNTKYNEKFKGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVGDRVVWHQQKPG |
| 1361 | (CDRs underlined) | KAPKALIYSSSHRYKGVPSRFSGSGSGTEFTLTISSLQPEDFAT |
| | | YFCQQFKSYPLTFGQGTKLEIK |

BKM0210

| | | |
|---|---|---|
| SEQ ID NO: | VH (CDRs underlined) | QVQLVQSGAEVKKPGSSVKVSCKASGHDFKLTYIHWVRQA |
| 1360 | | PGQGLEWMGRISAGSGNTKYNEKFKGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1353 | (CDRs underlined) | KAPKALIYSSSHRYKGVPSRFSGSGSGTEFTLTISSLQPEDFAT |
| | | YFCQQFKSYPLTFGQGTKLEIK |

BJM0816

| | | |
|---|---|---|
| SEQ ID NO: 9 | VH (CDRs underlined) | QVQLVQSGAEVKKPGSSVKVSCKASGYSFTTYYIHWVRQAP |
| | | GQGLEWMGWFFPGSGNIKYNEKFKGRVTITADTSTSTAYME |
| | | LSSLRSEDTAVYYCAGSYYSYDVLDYWGQGTTVTVSS |
| SEQ ID NO: 10 | VL | DIQMTQSPSFLSASVGDRVTITCKASQNVGINVVWHQQKPG |
| | (CDRs underlined) | KAPKALIYSSSHRYSGVPSRFSGSGSGTEFTLTISSLQPEDFAT |
| | | YFCQQFKSYPLTFGQGTKLEIK |

TABLE 2
Amino acid and nucleotide sequences for murine and humanized
antibody molecules which bind to TCRVB 12, e.g., TCRVB 12-3 or TCRVB 12-4.
The antibody molecules include murine mAb Antibody B and humanized mAb
Antibody B-H.1 to B-H.6. The amino acid the heavy and light chain
CDRs, and the amino acid and nucleotide sequences of the heavy and light chain variable
regions, and the heavy and light chains are shown.
Antibody B (murine), also referred to as 16G8

| | | |
|---|---|---|
| SEQ ID NO: | HC CDR1 | GFTFSNFGMH |
| 17 | (Combined) | |
| SEQ ID NO: | HC CDR2 | YISSGSSTIYYADTLKG |
| 18 | (Combined) | |
| SEQ ID NO: | HC CDR3 | RGEGAMDY |
| 19 | (Combined) | |
| SEQ ID NO: | HC CDR1 (Kabat) | NFGMH |
| 57 | | |
| SEQ ID NO: | HC CDR2 (Kabat) | YISSGSSTIYYADTLKG |
| 58 | | |
| SEQ ID NO: | HC CDR3 (Kabat) | RGEGAMDY |
| 59 | | |
| SEQ ID NO: | HC CDR1 (Chothia) | GFTFSNF |
| 60 | | |
| SEQ ID NO: | HC CDR2 (Chothia) | SSGSST |
| 61 | | |
| SEQ ID NO: | HC CDR3 (Chothia) | RGEGAMDY |
| 62 | | |
| SEQ ID NO: | VH | DVQLVESGGGLVQPGGSRKLSCAASGFTFSNFGMHWVRQAPD |
| 15 | | KGLEWVAYISSGSSTIYYADTLKGRFTISRDNPKNTLFLQMTSL |
| | | RSEDTAMYYCARRGEGAMDYWGQGTSVTVSS |
| SEQ ID NO: | LC CDR1 | RASSSVNYIY |
| 20 | (Combined) | |
| SEQ ID NO: | LC CDR2 | YTSNLAP |
| 21 | (Combined) | |
| SEQ ID NO: | LC CDR3 | QQFTSSPFT |
| 22 | (Combined) | |
| SEQ ID NO: | LC CDR1 (Kabat) | RASSSVNYIY |
| 63 | | |
| SEQ ID NO: | LC CDR2 (Kabat) | YTSNLAP |
| 64 | | |
| SEQ ID NO: | LC CDR3 (Kabat) | QQFTSSPFT |
| 65 | | |
| SEQ ID NO: | LC CDR1 (Chothia) | RASSSVNYIY |
| 66 | | |
| SEQ ID NO: | LC CDR2 (Chothia) | YTSNLAP |
| 67 | | |
| SEQ ID NO: | LC CDR3 (Chothia) | QQFTSSPFT |
| 68 | | |
| SEQ ID NO: | VL | ENVLTQSPAIMSASLGEKVTMSCRASSSVNYIYWYQQKSDASP |
| 16 | | KLWIYYTSNLAPGVPTRESGSGSGNSYSLTISSMEGEDAATYY |
| | | CQQFTSSPFTFGSGTKLEIK |

Antibody B humanized (B-H)
Antibody B-H.1A HC-1

| | | |
|---|---|---|
| SEQ ID NO: | HC CDR1 | GFTFSNFGMH |
| 17 | (Combined) | |
| SEQ ID NO: | HC CDR2 | YISSGSSTIYYADTLKG |
| 18 | (Combined) | |
| SEQ ID NO: | HC CDR3 | RGEGAMDY |
| 19 | (Combined) | |
| SEQ ID NO: | VH | EVQLVESGGGLVQPGGSLRLSCAASGFTFSNFGMHWVRQAPG |
| 3438 | | KGLEWVSYISSGSSTIYYADTLKGRFTISRDNAKNSLYLQMNS |
| | | LRAEDTAVYYCARRGEGAMDYWGQGTTVTVSS |
| SEQ ID NO: | DNA VH | GAGGTGCAGCTGGTTGAATCTGGCGGAGGATTGGTTCAGCC |
| 31 | | TGGCGGCTCTCTGAGACTGTCTTGTGCCGCTTCTGGCTTCAC |
| | | CTTCTCCAACTTCGGCATGCACTGGGTCCGACAGGCCCCTG |
| | | GAAAAGGACTGGAATGGGTGTCCTACATCTCCTCCGGCTCC |
| | | TCCACCATCTACTACGCTGACACCCTGAAGGGCAGATTCAC |
| | | CATCTCTCGGGACAACGCCAAGAACTCCCTGTACCTGCAGA |
| | | TGAACAGCCTGAGAGCCGAGGACACCGCCGTGTACTACTGT |
| | | GCTAGAAGAGGCGAGGGCGCCATGGATTATTGGGGCCAGG |
| | | GAACCACAGTGACCGTGTCTAGC |

Antibody B-H.1B HC-2

| | | |
|---|---|---|
| SEQ ID NO: | HC CDR1 | GFTFSNFGMH |
| 17 | (Combined) | |
| SEQ ID NO: | HC CDR2 | YISSGSSTIYYADTLKG |
| 18 | (Combined) | |
| SEQ ID NO: | HC CDR3 | RGEGAMDY |
| 19 | (Combined) | |
| SEQ ID NO: | VH | EVQLVESGGGLVQPGGSLRLSCAASGFTFSNFGMHWVRQAPG |
| 25 | | KGLEWVSYISSGSSTIYYADTLKGRFTISRDNSKNTLYLQMNSL |
| | | RAEDTAVYYCARRGEGAMDYWGQGTTVTVSS |
| SEQ ID NO: | DNA VH | GAGGTGCAGCTGGTTGAATCTGGCGGAGGATTGGTTCAGCC |
| 32 | | TGGCGGCTCTCTGAGACTGTCTTGTGCCGCTTCTGGCTTCAC |
| | | CTTCTCCAACTTCGGCATGCACTGGGTCCGACAGGCCCCTG |
| | | GAAAAGGACTGGAATGGGTGTCCTACATCTCCTCCGGCTCC |
| | | TCCACCATCTACTACGCTGACACCCTGAAGGGCAGATTCAC |
| | | CATCAGCCGGGACAACTCCAAGAACACCCTGTACCTGCAGA |
| | | TGAACTCCCTGAGAGCCGAGGACACCGCCGTGTACTACTGT |
| | | GCTAGAAGAGGCGAGGGCGCCATGGATTATTGGGGCCAGG |
| | | GAACCACAGTGACCGTGTCTAGC |

Antibody B-H.1C HC-3

| | | |
|---|---|---|
| SEQ ID NO: | HC CDR1 | GFTFSNFGMH |
| 17 | (Combined) | |
| SEQ ID NO: | HC CDR2 | YISSGSSTIYYADTLKG |
| 18 | (Combined) | |
| SEQ ID NO: | HC CDR3 | RGEGAMDY |
| 19 | (Combined) | |
| SEQ ID NO: | VH | QVQLVESGGGVVQPGRSLRLSCAASGFTFSNFGMHWVRQAPG |
| 23 | | KGLEWVAYISSGSSTIYYADTLKGRFTISRDNSKNTLYLQMNS |
| | | LRAEDTAVYYCARRGEGAMDYWGQGTTVTVSS |
| SEQ ID NO: | DNA VH | CAGGTGCAGCTGGTGGAATCTGGTGGCGGAGTTGTGCAGCC |
| 33 | | TGGCAGATCCCTGAGACTGTCTTGTGCCGCCTCTGGCTTCAC |
| | | CTTCTCCAACTTCGGCATGCACTGGGTCCGACAGGCCCCTG |
| | | GAAAAGGATTGGAGTGGGTCGCCTACATCTCCTCCGGCTCC |
| | | TCCACCATCTACTACGCTGACACCCTGAAGGGCAGATTCAC |
| | | CATCAGCCGGGACAACTCCAAGAACACCCTCTACCTGCAGA |
| | | TGAACTCCCTGAGAGCCGAGGACACCGCCGTGTACTACTGT |
| | | GCTAGAAGAGGCGAGGGCGCCATGGATTATTGGGGCCAGG |
| | | GAACCACAGTGACCGTGTCTAGC |

Antibody B-H.1D LC-1

| | | |
|---|---|---|
| SEQ ID NO: | LC CDR1 | RASSSVNYIY |
| 20 | (Combined) | |
| SEQ ID NO: | LC CDR2 | YTSNLAP |
| 21 | (Combined) | |
| SEQ ID NO: | LC CDR3 | QQFTSSPFT |
| 22 | (Combined) | |
| SEQ ID NO: | VL | DNQLTQSPSFLSASVGDRVTITCRASSSVNYIYWYQQKPGKAP |
| 26 | | KLLIYYTSNLAPGVPSRFSGSGSGNEYTLTISSLQPEDFATYYC |
| | | QQFTSSPFTFGQGTKLEIK |
| SEQ ID NO: | DNA VL | GATAACCAGCTGACCCAGTCTCCTAGCTTCCTGTCTGCCTCT |
| 34 | | GTGGGCGACAGAGTGACAATTACCTGCCGGGCCTCCTCCTC |
| | | CGTGAACTACATCTACTGGTATCAGCAGAAGCCCGGCAAGG |
| | | CCCCTAAGCTGCTGATCTACTACACCTCCAATCTGGCCCCTG |
| | | GCGTGCCCTCTAGATTTTCCGGATCTGGCTCCGCCAACGAGT |
| | | ATACCCTGACAATCTCCAGCCTGCAGCCTGAGGACTTCGCC |
| | | ACCTACTACTGCCAGCAGTTCACCTCCTCTCCATTCACCTTT |
| | | GGCCAGGGCACCAAGCTGGAAATCAAA |

Antibody B-H.1E LC-2

| | | |
|---|---|---|
| SEQ ID NO: | LC CDR1 | RASSSVNYIY |
| 20 | (Combined) | |
| SEQ ID NO: | LC CDR2 | YTSNLAP |
| 21 | (Combined) | |
| SEQ ID NO: | LC CDR3 | QQFTSSPFT |
| 22 | (Combined) | |
| SEQ ID NO: | VL | DNQLTQSPSSLSASVGDRVTITCRASSSVNYIYWYQQKPGKAP |
| 27 | | KLLIYYTSNLAPGVPSRFSGSGSGNDYTLTISSLQPEDFATYYC |
| | | QQFTSSPFTFGQGTKLEIK |
| SEQ ID NO: | DNA VL | ATAACCAGCTGACCCAGTCTCCTTCCAGCCTGTCTGCTTCTG |
| 35 | | TGGGCGACAGAGTGACAATTACCTGCCGGGCCTCCTCCTCC |
| | | GTGAACTACATCTACTGGTATCAGCAGAAGCCCGGCAAGGC |
| | | CCCTAAGCTGCTGATCTACTACACCTCCAATCTGGCCCCTGG |
| | | CGTGCCCTCTAGATTTTCCGGATCTGGCTCCGGCAACGACTA |
| | | TACCCTGACAATCTCCAGCCTGCAGCCTGAGGACTTCGCCA |
| | | CCTACTACTGCCAGCAGTTCACCTCCTCTCCATTCACCTTTG |
| | | GCCAGGGCACCAAGCTGGAAATCAAA |

Antibody B-H.1F LC-3

| | | |
|---|---|---|
| SEQ ID NO: | LC CDR1 | RASSSVNYIY |
| 20 | (Combined) | |
| SEQ ID NO: | LC CDR2 | YTSNLAP |
| 21 | (Combined) | |
| SEQ ID NO: | LC CDR3 | QQFTSSPFT |
| 22 | (Combined) | |
| SEQ ID NO: | VL | ENVLTQSPATLSVSPGERATLSCRASSSVNYIYWYQQKPGQAP |
| 28 | | RLLIYYTSNLAPGIPARFSGSGSGNEYTLTISSLQSEDFAVYYCQ |
| | | QFTSSPFTFGQGTKLEIK |
| SEQ ID NO: | DNA VL | GAGAATGTGCTGACCCAGTCTCCTGCCACACTGTCTGTTAGC |
| 36 | | CCTGGCGAGAGAGCTACCCTGAGCTGCAGAGCCTCTTCCTC |
| | | CGTGAACTACATCTACTGGTATCAGCAGAAGCCCGGCCAGG |
| | | CTCCTAGACTGCTGATCTACTACACCTCCAATCTGGCCCCTG |
| | | GCATCCCTGCCAGATTTTCCGGATCTGGCTCCGGCAACGAG |
| | | TATACCCTGACCATCTCCAGCCTGCAGTCCGAGGACTTTGCT |
| | | GTGTACTATTGCCAGCAGTTCACAAGCAGCCCTTTCACCTTT |
| | | GGCCAGGGCACCAAGCTGGAAATCAAA |

Antibody B-H.1G LC-4

| | | |
|---|---|---|
| SEQ ID NO: | LC CDR1 | RASSSVNYIY |
| 20 | (Combined) | |
| SEQ ID NO: | LC CDR2 | YTSNLAP |
| 21 | (Combined) | |
| SEQ ID NO: | LC CDR3 | QQFTSSPFT |
| 22 | (Combined) | |
| SEQ ID NO: | VL | QNVLTQPPSASGTPGQRVTISCRASSSVNYIYWYQQLPGTAPK |
| 29 | | LLIYYTSNLAPGVPDRFSGSGSGNSYSLAISGLRSEDEADYYCQ |
| | | QFTSSPFTFGTGTKVTVL |
| SEQ ID NO: | DNA VL | CAGAATGTGCTGACCCAACCTCCTTCCGCCTCTGGCACACCT |
| 37 | | GGACAGAGAGTGACAATCTCCTGCCGGGCCTCCTCCTCCGT |
| | | GAACTACATCTACTGGTATCAGCAGCTGCCCGGCACCGCTC |
| | | CTAAACTGCTGATCTACTACACCTCCAATCTGGCCCCTGGCG |
| | | TGCCCGATAGATTTTCCGGATCTGGCTCCGGCAACTCCTACA |
| | | GCCTGGCTATCTCTGGCCTGAGATCTGAGGACGAGGCCGAC |
| | | TACTACTGCCAGCAGTTCACCTCCTCTCCATTCACCTTTGGC |
| | | ACCGGCACCAAAGTGACAGTTCTT |

Antibody B-H.1H LC-5

| | | |
|---|---|---|
| SEQ ID NO: | LC CDR1 | RASSSVNYIY |
| 20 | (Combined) | |
| SEQ ID NO: | LC CDR2 | YTSNLAP |
| 21 | (Combined) | |
| SEQ ID NO: | LC CDR3 | QQFTSSPFT |
| 22 | (Combined) | |
| SEQ ID NO: | VL | SNELTQPPSVSVSPGQTARITCRASSSVNYIYWYQQKSGQAPV |
| 30 | | LVIYYTSNLAPGIPERFSGSGSGNMYTLTISGAQVEDEADYYCQ |
| | | QFTSSPFTFGTGTKVTVL |
| SEQ ID NO: | DNA VL | TCTAATGAGCTGACCCAGCCTCCTTCCGTGTCCGTGTCTCCT |
| 38 | | GGACAGACCGCCAGAATTACCTGCCGGGCCTCCTCCTCCGT |
| | | GAACTACATCTACTGGTATCAGCAGAAGTCCGGCCAGGCTC |
| | | CTGTGCTCGTGATCTACTACACCTCCAATCTGGCCCCTGGCA |
| | | TCCCTGAGAGATTCTCCGGATCTGGCTCCGGCAACATGTAC |
| | | ACCCTGACCATCTCTGGCGCCCAGGTGGAAGATGAGGCCGA |
| | | CTACTACTGCCAGCAGTTCACCTCCTCTCCATTCACCTTTGG |
| | | CACCGGCACCAAAGTGACAGTTCTT |

Antibody B-H.1

| | | |
|---|---|---|
| SEQ ID NO: | Chain1: Fc only | METDTLLLWVLLLWVPGSTGDKTHTCPPCPAPELLGGPSVFLF |
| 3280 | | PPKPKDTLMISRTPEVTCVVVDVSHEDPEVKFNWYVDGVEVH |
| | | NAKTKPREEQYNSTYRVVSVLTVLHQDWLNGKEYKCKVSNK |
| | | ALPAPIEKTISKAKGQPREPQVYTLPPCREEMTKNQVSLWCLV |
| | | KGFYPSDIAVEWESNGQPENNYKTTPPVLDSDGSFFLYSKLTV |
| | | DKSRWQQGNVFSCSVMHEALHNRFTQKSLSLSPGK |
| SEQ ID NO: | Chain2: humanized | METDTLLLWVLLLWVPGSTGEVQLVESGGGLVQPGGSLRLSC |
| 3281 | B-H scFv | AASGFTFSNFGMHWVRQAPGKGLEWVSYISSGSSTIYYADTLK |
| | | GRFTISRDNSKNTLYLQMNSLRAEDTAVYYCARRGEGAMDY |
| | | WGQGTTVTVSSGGGGSGGGGSGGGGSGGGGSDNQLTQSPSFL |
| | | SASVGDRVTITCRASSSVNYIYWYQQKPGKAPKLLIYYTSNLA |
| | | PGVPSRFSGSGSGNEYTLTISSLQPEDFATYYCQQFTSSPFTFGQ |
| | | GTKLEIKGGGGSDKTHTCPPCPAPELLGGPSVFLFPPKPKDTLM |
| | | ISRTPEVTCVVVDVSHEDPEVKFNWYVDGVEVHNAKTKPREE |
| | | QYNSTYRVVSVLTVLHQDWLNGKEYKCKVSNKALPAPIEKTIS |
| | | KAKGQPREPQVCTLPPSREEMTKNQVSLSCAVKGFYPSDIAVE |
| | | WESNGQPENNYKTTPPVLDSDGSFFLVSKLTVDKSRWQQGNV |
| | | FSCSVMHEALHNHYTQKSLSLSPGKGGGGSGGGGSGLNDIFEA |
| | | QKIEWHE |
| SEQ ID NO: | scFv | EVQLVESGGGLVQPGGSLRLSCAASGFTFSNFGMHWVRQAPG |
| 3642 | | KGLEWVSYISSGSSTIYYADTLKGRFTISRDNSKNTLYLQMNSL |
| | | RAEDTAVYYCARRGEGAMDYWGQGTTVTVSSGGGGSGGGG |
| | | SGGGGSGGGGSDNQLTQSPSFLSASVGDRVTITCRASSSVNYIY |
| | | WYQQKPGKAPKLLIYYTSNLAPGVPSRFSGSGSGNEYTLTISSL |
| | | QPEDFATYYCQQFTSSPFTFGQGTKLEIK |

Antibody B-H.2

| | | |
|---|---|---|
| SEQ ID NO: | scFv | EVQLVESGGGLVQPGGSLRLSCAASGFTFSNFGMHWVRQAPG |
| 1338 | | KGLEWVSYISSGSSTIYYADTLKGRFTISRDNSKNTLYLQMNSL |
| | | RAEDTAVYYCARRGEGAMDYWGQGTTVTVSSGGGGSGGGG |
| | | SGGGGSGGGGSDNQLTQSPSSLSASVGDRVTITCRASSSVNYIY |
| | | WYQQKPGKAPKLLIYYTSNLAPGVPSRFSGSGSGNDYTLTISSL |
| | | QPEDFATYYCQQFTSSPFTFGQGTKLEIK |

Antibody B-H.3

| | | |
|---|---|---|
| SEQ ID NO: | scFv | EVQLVESGGGLVQPGGSLRLSCAASGFTFSNFGMHWVRQAPG |
| 1339 | | KGLEWVSYISSGSSTIYYADTLKGRFTISRDNSKNTLYLQMNSL |
| | | RAEDTAVYYCARRGEGAMDYWGQGTTVTVSSGGGGSGGGG |
| | | SGGGGSGGGGSSNELTQPPSVSVSPGQTARITCRASSSVNYIYW |
| | | YQQKSGQAPVLVIYYTSNLAPGIPERFSGSGSGNMYTLTISGAQ |
| | | VEDEADYYCQQFTSSPFTFGTGTKVTVL |

Antibody B-H.4 (BJM1112)

| | | |
|---|---|---|
| SEQ ID NO: | scFv | QVQLVESGGGVVQPGRSLRLSCAASGFTFSNFGMHWVRQ |
| 1340 | (bolded section is | APGKGLEWVAYISSGSSTIYYADTLKGRFTISRDNSKNTLYL |
| | VH, italicized section | QMNSLRAEDTAVYYCARRGEGAMDYWGQGTTVTVSSGGG |
| | is VL, CDRs are | GSGGGGSGGGGSGGGGSDNQLTQSPSFLSASVGDRVTITCRASSS |
| | underlined) | VNYIYWYQQKPGKAPKLLIYYTSNLAPGVPSRFSGSGSGNEYTLTISS |
| | | LQPEDFATYYCQQFTSSPFTFGQGTKLEIK |

Antibody B-H.5

| | | |
|---|---|---|
| SEQ ID NO: | scFv | QVQLVESGGGVVQPGRSLRLSCAASGFTFSNFGMHWVRQAPG |
| 1341 | | KGLEWVAYISSGSSTIYYADTLKGRFTISRDNSKNTLYLQMNS |
| | | LRAEDTAVYYCARRGEGAMDYWGQGTTVTVSSGGGGSGGG |
| | | GSGGGGSGGGGSDNQLTQSPSSLSASVGDRVTITCRASSSVNYI |
| | | YWYQQKPGKAPKLLIYYTSNLAPGVPSRFSGSGSGNDYTLTIS |
| | | SLQPEDFATYYCQQFTSSPFTFGQGTKLEIK |

Antibody B-H.6

| | | |
|---|---|---|
| SEQ ID NO: | scFv | QVQLVESGGGVVQPGRSLRLSCAASGFTFSNFGMHWVRQAPG |
| 1342 | | KGLEWVAYISSGSSTIYYADTLKGRFTISRDNSKNTLYLQMNS |
| | | LRAEDTAVYYCARRGEGAMDYWGQGTTVTVSSGGGGSGGG |
| | | GSGGGGSGGGGSSNELTQPPSVSVSPGQTARITCRASSSVNYIY |
| | | WYQQKSGQAPVLVIYYTSNLAPGIPERFSGSGSGNMYTLTISG |
| | | AQVEDEADYYCQQFTSSPFTFGTGTKVTVL |

TABLE 3
Constant region amino acid sequences of
human IgG heavy chains and human kappa light chain

| | | |
|---|---|---|
| Human kappa | LC | RTVAAPSVFI FPPSDEQLKS GTASVVCLLN NFYPREAKVQ WKVDNALQSG |
| constant region | | NSQESVTEQD SKDSTYSLSS TLILSKADYE KHKVYACEVT HQGLSSPVTK |
| SEQ ID NO: 39 | | SFNRGEC |
| Human | LC | RTVAAPSVFIFPPSDEQLKSGTASVVCLLNNFYPREAKVQWKVDNALQSGNSQ |
| Immunoglobulin | | ESVTEQDSKDSTYSLSSTLTLSKADYEKHKVYACEVTHQGLSSPVTKSFNRGE |
| kappa constant C | | C |
| region | | |
| SEQ ID NO: 3644 | | |
| IgG4 (S228P) | HC | ASTKGPSVFPLAPCSRSTSESTAALGCLVKDYFPEPVTVSWNSGALTSGVHTFP |
| mutant constant | | AVLQSSGLYSLSSVVTVPSSSLGTKTYTCNVDHKPSNTKVDKRVESKYGPPCPP |
| region (EU | | CPAPEFLGGPSVFLFPPKPKDTLMISRTPEVTCVVVDVSQEDPEVQFNWYVDG |
| Numbering) | | VEVHNAKTKPREEQFNSTYRVVSVLTVLHQDWLNGKEYKCKVSNKGLPSSIE |
| SEQ ID NO: 40 | | KTISKAKGQPREPQVYTLPPSQEEMTKNQVSLTCLVKGFYPSDIAVEWESNGQ |
| | | PENNYKTTPPVLDSDGSFFLYSRLTVDKSRWQEGNVFSCSVMHEALHNHYTQ |
| | | KSLSLSLG |
| IgG1 wild type | HC | ASTKGPSVFPLAPSSKSTSGGTAALGCLVKDYFPEPVTVSWNSGALTSGVHTFP |
| SEQ ID NO: 41 | | AVLQSSGLYSLSSVVTVPSSSLGTQTYICNVNHKPSNTKVDKRVEPKSCDKTHT |
| | | CPPCPAPELLGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALHNHY |
| | | TQKSLSLSPGK |
| IgG1 (N297A) | HC | ASTKGPSVFPLAPSSKSTSGGTAALGCLVKDYFPEPVTVSWNSGALTSGVHTFP |
| mutant constant | | AVLQSSGLYSLSSVVTVPSSSLGTQTYICNVNHKPSNTKVDKRVEPKSCDKTHT |
| region (EU | | CPPCPAPELLGGPSVFLFPPKPKDTLMISRTPEVTCVVVDVSHEDPEVKFNWYV |
| Numbering) | | DGVEVHNAKTKPREEQYASTYRVVSVLTVLHQDWLNGKEYKCKVSNKALPA |
| SEQ ID NO: 42 | | PIEKTISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALHNHY |
| | | TQKSLSLSPGK |
| IgM constant | HC | GSASAPTLFPLVSCENSPSDTSSVAVGCLAQDFLPDSITFSWKYKNNSDISSTRG |
| delta CDC | | FPSVLRGGKYAATSQVLLPSKDVMQGTDEHVVCKVQHPNGNKEKNVPLPVIA |
| (P311A, P313S) | | ELPPKVSVFVPPRDGFFGNPRKSKLICQATGFSPRQIQVSWLREGKQVGSGVTT |
| SEQ ID NO: 73 | | DQVQAEAKESGPTTYKVTSTLTIKESDWLGQSMFTCRVDHRGLTFQQNASSM |
| | | CVPDQDTAIRVFAIPPSFASIFLTKSTKLTCLVTDLTTYDSVTISWTRQNGEAVK |
| | | THTNISESHPNATFSAVGEASICEDDWNSGERFTCTVTHTDLASSLKQTISRPKG |
| | | VALHRPDVYLLPPAREQLNLRESATITCLVTGFSPADVFVQWMQRGQPLSPEK |
| | | YVTSAPMPEPQAPGRYFAHSILTVSEEEWNTGETYTCVVAHEALPNRVTERTV |
| | | DKSTGKPTLYNVSLVMSDTAGTCY |
| IgGA1 | HC | ASPTSPKVFPLSLCSTQPDGNVVIACLVQGFFPQEPLSVTWSESGQGVTARNFPP |
| SEQ ID NO: 74 | | SQDASGDLYTTSSQLTLPATQCLAGKSVTCHVKHYTNPSQDVTVPCPVPSTPPT |
| | | PSPSTPPTPSPSCCHPRLSLHRPALEDLLLGSEANLTCTLTGLRDASGVTFTWTP |
| | | SSGKSAVQGPPERDLCGCYSVSSVLPGCAEPWNHGKTFTCTAAYPESKTPLTA |
| | | TLSKSGNTFRPEVHLLPPPSEELALNELVTLTCLARGFSPKDVLVRWLQGSQEL |
| | | PREKYLTWASRQEPSQGTTTFAVTSILRVAAEDWKKGDTFSCMVGHEALPLAF |
| | | TQKTIDRLAGKPTHVNVSVVMAEVDGTCY |
| IgGA2 | HC | ASPTSPKVFPLSLDSTPQDGNVVVACLVQGFFPQEPLSVTWSESGQNVTARNFP |
| SEQ ID NO: 75 | | PSQDASGDLYTTSSQLTLPATQCPDGKSVTCHVKHYTNSSQDVTVPCRVPPPPP |
| | | CCHPRLSLHRPALEDLLLGSEANLTCTLTGLRDASGATFTWTPSSGKSAVQGPP |
| | | ERDLCGCYSVSSVLPGCAQPWNHGETFTCTAAHPELKTPLTANITKSGNTFRPE |
| | | VHLLPPPSEELALNELVTLTCLARGFSPKDVLVRWLQGSQELPREKYLTWASR |
| | | QEPSQGTTTYAVTSILRVAAEDWKKGETFSCMVGHEALPLAFTQKTIDRMAG |
| | | KPTHINVSVVMAEADGTCY |
| Human Ig_J chain | HC | MKNHLLFWGVLAVFIKAVHVKAQEDERIVLVDNKCKCARITSRIIRSSEDPNED |
| SEQ ID NO: 76 | | IVERNIRIIVPLNNRENISDPTSPLRTRFVYHLSDLCKKCDPTEVELDNQIVTATQ |
| | | SNICDEDSATETCYTYDRNKCYTAVVPLVYGGETKMVETALTPDACYPD |
| Human | HC | ASTKGPSVFPLAPSSKSTSGGTAALGCLVKDYFPEPVTVSWNSGALTSGVHTFP |
| Immunoglobulin | | AVLQSSGLYSLSSVVTVPSSSLGTQTYICNVNHKPSNTKVDKKVEPKSCDKTHT |
| heavy chain | | CPPCPAPELLGGPSVFLFPPKPKDTLMISRTPEVTCVVVDVSHEDPEVKFNWYV |
| SEQ ID NO: 3645 | | DGVEVHNAKTKPREEQYASTYRVVSVLTVLHQDWLNGKEYKCKVSNKALPA |
| | | PIEKTISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALHNHY |
| | | TQKSLSLSPGK |
| Human | HC | ASTKGPSVFPLAPSSKSTSGGTAALGCLVKDYFPEPVTVSWNSGALTSGVHTFP |
| Immunoglobulin | | AVLQSSGLYSLSSVVTVPSSSLGTQTYICNVNHKPSNTKVDKKVEPKSCDKTHT |
| heavy chain | | CPPCPAPELLGGPSVFLFPPKPKDTLMISRTPEVTCVVVDVSHEDPEVKENWYV |
| SEQ ID NO: 3646 | | DGVEVHNAKTKPREEQYASTYRVVSVLTVLHQDWLNGKEYKCKVSNKALPA |
| | | PIEKTISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALHNHY |
| | | TQKSLSLSPGKEPEA |
| Human | HC | ASTKGPSVFPLAPSSKSTSGGTAALGCLVKDYFPEPVTVSWNSGALTSGVHTFP |
| Immunoglobulin | | AVLQSSGLYSLSSVVTVPSSSLGTQTYICNVNHKPSNTKVDKRVEPKSCDKTHT |
| heavy chain | | CPPCPAPELLGGPSVFLFPPKPKDTLMISRTPEVTCVVVDVSHEDPEVKFNWYV |
| SEQ ID NO: 3647 | | DGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALHNHY |
| | | TQKSLSLSPGK |
| Human Fc Knob | HC | DKTHTCPPCPAPELLGGPSVFLFPPKPKDTLMISRTPEVTCVVVDVSHEDPEVK |
| cys N297A | | FNWYVDGVEVHNAKTKPREEQYASTYRVVSVLTVLHQDWLNGKEYKCKVSNK |
| SEQ ID NO: 3648 | | ALPAPIEKTISKAKGQPREPQVYTLPPCREEMTKNQVSLWCLVKGFYPSDIAVE |
| | | WESNGQPENNYKTTPPVLDSDGSFFLYSKLTVDKSRWQQGNVFSCSVMHEAL |
| | | HNHYTQKSLSLSPGK |
| Human IgG1 hole | HC | ASTKGPSVFPLAPSSKSTSGGTAALGCLVKDYFPEPVTVSWNSGALTSGVHTFP |
| cys N297A | | AVLQSSGLYSLSSVVTVPSSSLGTQTYICNVNHKPSNTKVDKRVEPKSCDKTHT |
| SEQ ID NO: 3649 | | CPPCPAPELLGGPSVFLFPPKPKDTLMISRTPEVTCVVVDVSHEDPEVKFNWYV |
| | | DGVEVHNAKTKPREEQYASTYRVVSVLTVLHQDWLNGKEYKCKVSNKALPA |
| | | PIEKTISKAKGQPREPQVCTLPPSREEMTKNQVSLSCAVKGFYPSDIAVEWESN |
| | | GQPENNYKTTPPVLDSDGSFFLVSKLTVDKSRWQQGNVFSCSVMHEALHNHY |
| | | TQKSLSLSPGK |

TABLE 4
Exemplary Fc KiH mutations and optional Cysteine mutations

| Position | Knob Mutation | Hole Mutation |
|---|---|---|
| T366 | T366W | T366S |
| L368 | — | L368A |
| Y407 | — | Y407V |

Additional Cysteine Mutations to
form a stabilizing disulfide bridge

| Position | Knob CH3 | Hole CH3 |
|---|---|---|
| S354 | S354C | — |
| Y349 | — | Y349C |

TABLE 5
CRS grading

| | |
|---|---|
| Gr1 | Supportive care only |
| Gr2 | IV therapies +/− hospitalization. |
| Gr3 | Hypotension requiring IV fluids or low-dose vasoactives or |
| | hypoxemia requiring oxygen, CPAP, or BIPAP. |
| Gr4 | Hypotension requiring high-dose vasoactives or hypoxemia |
| | requiring mechanical ventilation. |
| Gr 5 | Death |

TABLE 6
CTCAE v 4.0 CRS grading scale

| CRS grade | Characteristics |
|---|---|
| Grade 1 | Mild; No infusion interruption; No intervention |
| Grade 2 | Infusion interruption indicated but responds promptly to |
| | symptomatic treatment (e.g., antihistamines, NSAIDS, |
| | narcotics, IV fluids); prophylactic medications |
| | indicated for <= 24 hrs |
| Grade 3 | Prolonged (e.g., not rapidly responsive to symptomatic |
| | medications and/or brief interruption of infusion); |
| | recurrence of symptoms following initial improvement; |
| | hospitalization indicated for clinical sequelae (e.g., |
| | renal impairment, pulmonary infiltrates) |
| Grade 4 | Life threatening consequences; |
| | pressor or ventilator support |

TABLE 7
NCI CRS grading scale

| CRS grade | Characteristics |
|---|---|
| Grade 1 | Symptoms are not life threatening and require |
| | symptomatic treatment only; e.g., fever, nausea, fatigue, |
| | headache, myalgias, malaise |
| Grade 2 | Symptoms require and respond to moderate intervention; |
| | Oxygen requirement <40% of hypotension responsive to |
| | fluids or low dose pressors or Grade 2 organ toxicity |
| Grade 3 | Symptoms require and respond to aggressive intervention; |
| | Oxygen requirement >=40% or Hypotension requiring |
| | high dose or multiple pressors or grade 3 organ toxicity |
| | or grade 4 transaminitis |
| Grade 4 | Life threatening symptoms Requirement for ventilator |
| | support or Grade 4; organ toxicity |
| | (excluding transaminitis) |

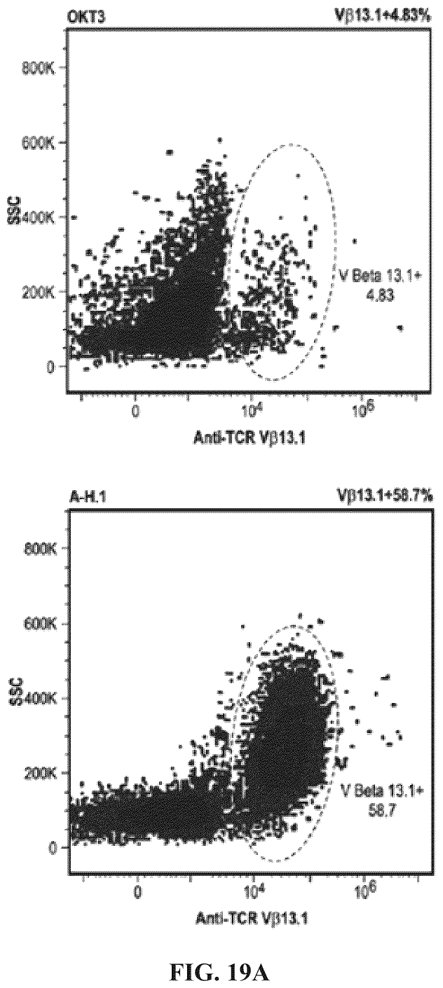

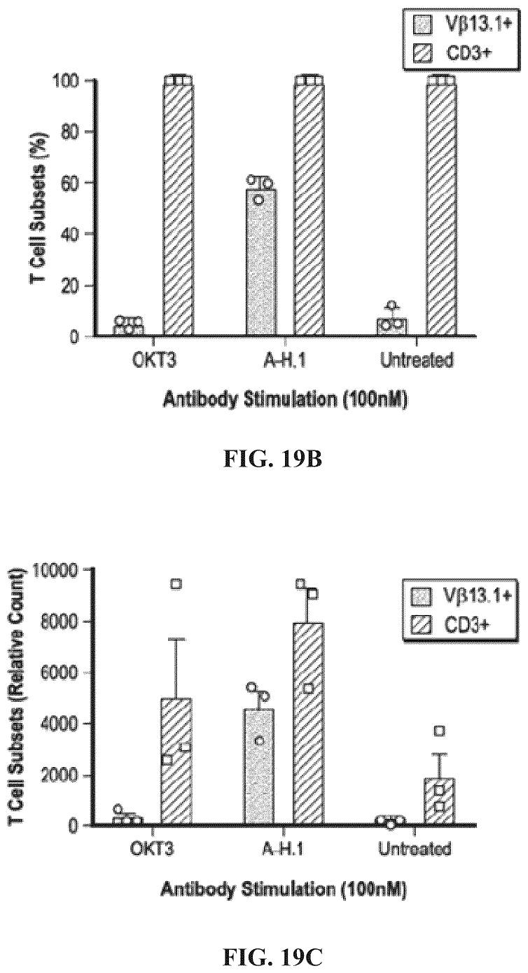

TABLE 8A
List of TCRβV subfamilies and subfamily members

| Reference | | |
|---|---|---|
| in FIG. 19 | Subfamily | Subfamily members |
| A | TCRβ V6 | TCRβ V6-4*01, TCRβ V6-4*02, |
| | Also referred | TCRβ V6-9*01, TCRβ V6-8*01, |
| | to as: TCR | TCRβ V6-5*01, TCRβ V6-6*02, |
| | VB 13.1 | TCRβ V6-6*01, TCRβ V6-2*01, |
| | | TCRβ V6-3*01 or TCRβ V6-1*01. |
| B | TCRβ V10 | TCRβ V10-1*01, TCRβ V10-1*02, |
| | Also referred | TCRβ V10-3*01 or TCRβ V10-2*01 |
| | to as: TCRβ | |
| | V12 | |
| C | TCRβ V12 | TCRβ V12-4*01, TCRβ V12-3*01, or |
| | Also referred | TCRβ V12-5*01 |
| | to as: TCRβ | |
| | V8.1 | |
| D | TCRβ V5 | TCRβ V5-5*01, TCRβ V5-6*01, TCRβ |
| | | V5-4*01, TCRβ V5-8*01, TCRβ V5-1*01 |
| E | TCRβ V7 | TCRβ V7-7*01, TCRβ V7-6*01, |
| | | TCRβ V7 -8*02, TCRβ V7 -4*01, |
| | | TCRβ V7-2*02, TCRβ V7-2*03, |
| | | TCRβ V7-2*01, TCRβ V7-3*01, |
| | | TCRβ V7-9*03, or TCRβ V7-9*01 |
| F | TCRβ V11 | TCRβ V11-1*01, TCRβ V11-2*01 or |
| | | TCRβ V11-3*01 |
| G | TCRβ V14 | TCRβ V14*01 |
| H | TCRβ V16 | TCRβ V16*01 |
| I | TCRβ V18 | TCRβ V18*01 |
| J | TCRβ V9 | TCRβ V9*01 or TCRβ V9*02 |
| K | TCRβ V13 | TCRβ V13*01 |
| L | TCRβ V4 | TCRβ V4-2*01, TCRβ V4-3*01, or |
| | | TCRβ V4-1*01 |
| M | TCRβ V3 | TCRβ V3-1*01 |
| N | TCRβ V2 | TCRβ V2*01 |
| O | TCRβ V15 | TCRβ V15*01 |
| P | TCRβ V30 | TCRβ V30*01, or TCRβ V30*02 |
| Q | TCRβ V19 | TCRβ V19*01, or TCRβ V19*02 |
| R | TCRβ V27 | TCRβ V27*01. |
| S | TCRβ V28 | TCRβ V28*01. |
| T | TCRβ V24 | TCRβ V24-1*01 |
| U | TCRβ V20 | TCRβ V20-1*01, or TCRβ V20-1*02 |
| V | TCRβ V25 | TCRβ V25-1*01 |
| W | TCRβ V29 | TCRβ V29-1*01 |

TABLE 8B
Additional TCRβV subfamilies
Subfamily

TCRβ V1
TCRβ V17
TCRβ V21
TCRβ V23
TCRβ V26

TABLE 10A
Exemplary anti-TCRβV antibody molecules

| TCRvβ | TCRvβ | Reagents monoclonal antibodies | | |
|---|---|---|---|---|
| gene name | allele name | Clone name and Specificity | Company product | Isotype |
| TCRvβ2 | TCRvβ2*01 | IsMMU 546 (TCRvβV2) | Serotec V BETA 22 | Mouse IgG1 |
| | TCRvβ2*02 | | Coulter Vbeta22 | |
| | TCRvβ2*03 | | | |
| TCRvβ3-1 | TCRvβ3-1*01 | FIN9 (TCRvβ3-1) | Serotec Vbeta9 | Mouse IgG2a |
| | | | Coulter Vbeta9 | |
| | TCRvβ3-1*02 | AMKB1-2 (TCRvβ3-1) | BD Biosciences Vbeta9 | Mouse IgG1 |
| TCRvβ4-1 | TCRvβ4-1*01 | ZOE (TCRvβ4-1, | Serotec V BETA 7 | Mouse IgG2a |
| | | TCRvβ4-2, TCRvβ4-3) | Coulter Vbeta7 | |
| | TCRvβ4-1*02 | 3G5 (TCRvβ4-1) | Pierce EndogenV beta 7.1 | Mouse IgG2b |
| TCRvβ4-2 | TCRvβ4-2*01 | ZOE (TCRvβ4-1, | Serotec V BETA 7 | Mouse IgG2a |
| | TCRvβ4-2*02 | TCRvβ4-2, TCRvβ4-3) | Coulter Vbeta7 | |
| TCRvβ4-3 | TCRvβ4-3*01 | ZOE (TCRvβ4-1, | Serotec V BETA 7 | Mouse IgG2a |
| | TCRvβ4-3*02 | TCRvβ4-2, TCRvβ-3) | Coulter Vbeta 7 | |
| | TCRvβ4-3*03 | | | |
| | TCRvβ4-3*04 | ZIZOU4 (TCRvβ4-3) | Coulter Vbeta7.2 | Mouse IgG2a |
| TCRvβ5-1 | TCRvβ5-1*01 | IMMU157 (TCRvβ5-1) | Serotec Vbeta5.1 | Mouse IgG2a |
| | | | Coulter Vbeta5.1 | |
| | TCRvβ5-1*02 | LC4 (TCRvβ5-1) | Pierce Endogen V beta 5(c) | Mouse IgG1 |
| | | | BD Biosciences Vbeta5(c) | |
| TCRvβ5-4 | TCRvβ5-4*01 | | | |
| | TCRvβ5-4*02 | | | |
| | TCRvβ5-4*03 | | | |
| | TCRvβ5-4*04 | | | |
| TCRvβ5-5 | TCRvβ5-5*01 | 3D11 (TCRvβ5-5) | Serotec VBETA5.3 | Mouse IgG1 |
| | | | Coulter Vbeta5.3 | |
| | TCRvβ5-5*02 | 1C1 (TCRvβ5-5, TCRvβ5-6) | Pierce Endogen V beta 5(a) | Mouse IgG1 |
| | | | BD Biosciences Vbeta5(a) | |
| | TCRvβ5-5*03 | W112 (TCRvβ5-5) | Pierce Endogen V beta 5(b) | Mouse IgG1 |
| | | | Serotec V beta 5.2/5.3 | |
| | | | BD Biosciences Vbeta5(b) | |
| | | MH3-2 (TCRvβ5-5, | BD Biosciences Vbeta5 | Mouse IgG2a |
| | | TCRvβ5-6) | | |
| | | 4H11 (TM27) as disclosed | | |
| | | in U.S. Pat. No. 5,861,155 | | |
| TCRvβ5-6 | TCRvβ5-6*01 | 36213 (TCRvβ5-6) | Serotec Vbeta5.2 | Mouse IgG1 |
| | | 1C1 (TCRvβ5-5, | BD Biosciences Vbeta5(a) | Mouse IgG1 |
| | | TCRvβ5-6) | BD Biosciences Vbeta5 | Mouse IgG2a |
| | | MH3-2 (TCRvβ5-5, | | |
| | | TCRvβ5-6) | | |
| TCRvβ5-8 | TCRvβ5-8*01 | | | |
| | TCRvβ5-8*02 | | | |
| TCRvβ6-1 | TCRvβ6-1*01 | BAM13 (TCRvβ6-1, | Pierce Endogen V beta 13 | Mouse IgG1 |
| | | TCRvβ6-5) | BD Biosciences Vbeta13.1, | |
| | | | 13.3 | |
| TCRvβ6-2 | TCRvβ6-2*01 | H132 | Coulter Vbeta13.2 | Mouse IgG1 |
| TCRvβ6-3 | TCRvβ6-3*01 | | | |
| TCRvβ6-4 | TCRvβ6-4*01 | | | |
| | TCRvβ6-4*02 | | | |
| TCRvβ6-5 | TCRvβ6-5*01 | IMMU 222 (TCRvβ6-5, | Serotec V BETA 13.1 | Mouse IgG2b |
| | | TCRvβ6-6 and TCRvβ6-9) | Coulter Vbeta13.1 | |
| | | BAM13 (TCRvβ6-1, | Pierce Endogen V beta 13 | Mouse IgG1 |
| | | TCRvβ6-5) | BD Biosciences Vbeta13.1, | |
| | | | 13.3 | |
| TCRvβ6-6 | TCRvβ6-6*01 | JU-74 (TCRvβ6-6) | Serotec Vbeta13.6 | Mouse IgG1 |
| | TCRvβ6-6*02 | JU74.3 (TCRvβ6-6) | Coulter Vbeta13.6 | |
| | TCRvβ6-6*03 | IMMU 222 (TCRvβ6-5, | | |
| | TCRvβ6-6*04 | TCRvβ6-6 and TCRvβ6-9) | Serotec V BETA 13.1 | Mouse IgG2b |
| | TCRvβ6-6*05 | | Coulter Vbeta13.1 | |
| TCRvβ6-8 | TCRvβ6-8*01 | | | |
| TCRvβ6-9 | TCRvβ6-9*01 | IMMU 222 (TCRvβ6-5, | Serotec V BETA 13.1 | Mouse IgG2b |
| | | TCRvβ6-6 and TCRvβ6-9) | Coulter Vbeta13.1 | |
| TCRvβ7-2 | TCRvβ7-2*01 | OT145 (TCRvβ7-2) | Pierce Endogen V beta 6.7 | Mouse IgG1 |
| | TCRvβ7-2*02 | | BD Biosciences Vbeta6.7 | |
| | TCRvβ7-2*03 | | | |
| | TCRvβ7-2*04 | | | |
| TCRvβ7-3 | TCRvβ7-3*01 | | | |
| | TCRvβ7-3*04 | | | |
| | TCRvβ7-3*05 | | | |
| TCRvβ7-4 | TCRvβ7-4*01 | | | |
| TCRvβ7-6 | TCRvβ7-6*01 | | | |
| | TCRvβ7-6*02 | | | |
| TCRvβ7-7 | TCRvβ7-7*01 | | | |
| | TCRvβ7-7*02 | | | |
| TCRvβ7-8 | TCRvβ7-8*01 | | | |
| | TCRvβ7-8*02 | | | |
| | TCRvβ7-8*03 | | | |
| TCRvβ7-9 | TCRvβ7-9*01 | | | |
| | TCRvβ7-9*02 | | | |
| | TCRvβ7-9*03 | | | |
| | TCRvβ7-9*04 | | | |
| | TCRvβ7-9*05 | | | |
| | TCRvβ7-9*06 | | | |
| | TCRvβ7-9*07 | | | |
| TCRvβ9 | TCRvβ9*01 | BL37.2 (TCRvβ9) | Serotec Vbeta1 | Rat IgG1 |
| | TCRvβ9*02 | | Coulter Vbeta1 | |
| | TCRvβ9*03 | | | |
| TCRvβ10-1 | TCRvβ10-1*01 | S511 (TCRvβ10-1, | Pierce Endogen V beta 12 | Mouse IgG2b |
| | TCRvβ10-1*02 | TCRvβ10-2, TCRvβ10-3) | BD Biosciences Vbeta12 | |
| TCRvβ10-2 | TCRvβ10-2*01 | | | |
| | TCRvβ10-2*02 | | | |
| TCRvβ10-3 | TCRvβ10-3*01 | VER2.32.1 (TCRvβ10-3) | Serotec Vbeta12 | Mouse IgG2a |
| | | | Coulter Vbeta12 | |
| | TCRvβ10-3*02 | S511 (TCRvβ10-1, | | |
| | TCRvβ10-3*03 | TCRvβ10-2, TCRvβ10-3) | Pierce Endogen V beta 12 | Mouse IgG2b |
| | | | BD Biosciences Vbeta 12 | |
| | TCRvβ10-3*04 | | | |
| TCRvβ11-1 | TCRvβ11-1*01 | | | |
| TCRvβ11-2 | TCRvβ11-2*01 | IG125 (TCRvβ11-2) | Serotec Vbeta21.3 | Mouse IgG2a |
| | TCRvβ11-2*02 | | Coulter Vbeta21.3 | |
| | TCRvβ11-2*03 | | | |
| TCRvβ11-3 | TCRvβ11-3*01 | | | |
| | TCRvβ11-3*02 | | | |
| | TCRvβ11-3*03 | | | |
| | TCRvβ11-3*04 | | | |
| TCRvβ12-3 | TCRvβ12-3*01 | 56C5 (TCRvβ12-3, | Serotec Vbeta8.1/8.2 | Mouse IgG2a |
| | | TCRvβ12-4) | Coulter Vbeta8 | |
| TCRvβ12-4 | TCRvβ12-4*01 | 56C5.2 (TCRvβ12-3, | Pierce Endogen V beta 8(a) | Mouse IgG2b |
| | | TCRvβ12-4) | BD Biosciences Vbeta8 | |
| | TCRvβ12-4*02 | 16G8 (TCRvβ12-3, | Pierce Endogen V beta 8(b) | Mouse IgG2a |
| | | TCRvβ12-4) | BD Biosciences Vbeta8 | Mouse IgG2b |
| | | MX-6 (TCRvβ12-3, | | |
| | | TCRvβ12-4) | | |
| | | JR2 (TCRvβ12-3, | | |
| | | TCRvβ12-4, TCRvβ12-5) | | |
| TCRvβ12-5 | TCRvβ12-5*01 | JR2 (TCRvβ12-3, | BD Biosciences Vbeta8 | Mouse IgG2b |
| | | TCRvβ12-4, TCRvβ12-5) | | |
| TCRvβ13 | TCRvβ13*01 | AF-23 (TCRvβ13) | Serotec Vbeta23 | Mouse IgG1 |
| | TCRvβ13*02 | AF23 (TCRvβ13) | Coulter Vbeta23 | |
| | | AHUT7 (Vbeta23) | BD Biosciences Vbeta23 | |
| TCRvβ14 | TCRvβ14*01 | TAMAYA1.2 (TCRvβ14) | Serotec Vbeta16 | Mouse IgG1 |
| | TCRvβ14*02 | | Coulter Vbeta16 | |
| TCRvβ15 | TCRvβ15*01 | | | |
| | TCRvβ15*02 | | | |
| | TCRvβ15*03 | | | |
| TCRvβ16 | TCRvβ16*01 | | | |
| | TCRvβ16*03 | | | |
| TCRvβ18 | TCRvβ18*01 | BA62 (TCRvβ18) | Serotec V BETA 18 | Mouse IgG1 |
| | | BA62.6 (TCRvβ18) | Coulter Vbeta18 | |
| TCRvβ19 | TCRvβ19*01 | C1 (TCRvβ19) | Pierce Endogen V beta 17 | Mouse IgG1 |
| | | E17.5F3 (TCRvβ19) | BD Biosciences Vbeta17 | |
| | TCRvβ19*02 | E17.5F3.15.13 (TCRvβ19) | Serotec Vbeta17 | Mouse IgG1 |
| | TCRvβ19*03 | | Coulter Vbeta17 | |
| TCRvβ20-1 | TCRvβ20-1*01 | MPB2D5 (TCRvβ20-1) | Serotec VBETA2 | Mouse IgG1 |
| | TCRvβ20-1*02 | | Coulter Vbeta2 | |
| | TCRvβ20-1*03 | | | |
| | TCRvβ20-1*04 | | | |
| | TCRvβ20-1*05 | | | |
| | TCRvβ20-1*06 | | | |
| | TCRvβ20-1*07 | | | |
| TCRvβ24-1 | TCRvβ24-1*01 | | | |
| TCRvβ25-1 | TCRvβ25-1*01 | C21 (TCRvβ25-1) | Serotec V BETA 11 | Mouse IgG2a |
| | | | Coulter Vbeta11 | |
| TCRvβ27 | TCRvβ27*01 | CAS1.1.3 (TCRvβ27) | Serotec Vbeta14 | Mouse IgG1 |
| | | | Coulter Vbeta14 | |
| TCRvβ28 | TCRvβ28*01 | CH92 (TCRvβ28) | Serotec Vbeta3 | Mouse IgM |
| | | 8F10 (TCRvβ28) | Coulter Vbeta3 | |
| | | JOVI-3 (TCRvβ28) | Pierce Endogen V beta 3.1 | Mouse IgG1 |
| | | | BD Biosciences Vbeta3 | Mouse IgG2a |
| TCRvβ29-1 | TCRvβ29-1*01 | WJF24 | Coulter Vbeta4 | Rat IgM |
| | TCRvβ29-1*02 | | | |
| | TCRvβ29-1*03 | | | |
| TCRvβ30 | TCRvβ30*01 | ELL1.4 (TCRvβ30) | Serotec Vbeta20 | Mouse IgG1 |
| | TCRvβ30*02 | | Coulter Vbeta20 | |
| | TCRvβ30*04 | | | |
| | TCRvβ30*05 | | | |

TABLE 10B
Amino acid sequences for anti TCRβ V5 antibodies. Amino acid and
nucleotide sequences for murine and humanized antibody molecules
which bind to TCRVB 5 (e.g., TCRVB 5-5 or TCRVB 5-6). The amino
acid the heavy and light chain CDRs, and the amino acid and nucleotide
sequences of the heavy and light chain variable regions, and the
heavy and light chains are shown.
Murine antibody C, also referred to as 4H11

| | | |
|---|---|---|
| SEQ ID NO: | HC CDR1 (Kabat) | AYGVN |
| 1315 | | |
| SEQ ID NO: | HC CDR2 (Kabat) | MIWGDGNTDYNSALKS |
| 1316 | | |
| SEQ ID NO: | HC CDR3 (Kabat) | DRVTATLYAMDY |
| 1317 | | |
| SEQ ID NO: | HC CDR1 (Chothia) | GFSLTAY |
| 1318 | | |
| SEQ ID NO: | HC CDR2 (Chothia) | WGDGN |
| 1319 | | |
| SEQ ID NO: | HC CDR3 (Chothia) | DRVTATLYAMDY |
| 1317 | | |
| SEQ ID NO: | HC CDR1 (Combined) | GFSLTAYGVN |
| 1320 | | |
| SEQ ID NO: | HC CDR2 (Combined) | MIWGDGNTDYNSALKS |
| 1316 | | |
| SEQ ID NO: | HC CDR3 (Combined) | DRVTATLYAMDY |
| 1317 | | |
| SEQ ID NO: | LC CDR1 (Kabat) | SASQGISNYLN |
| 1321 | | |
| SEQ ID NO: | LC CDR2 (Kabat) | YTSSLHS |
| 1322 | | |
| SEQ ID NO: | LC CDR3 (Kabat) | QQYSKLPRT |
| 1323 | | |
| SEQ ID NO: | LC CDR1 (Chothia) | SASQGISNYLN |
| 1321 | | |
| SEQ ID NO: | LC CDR2 (Chothia) | YTSSLHS |
| 1322 | | |
| SEQ ID NO: | LC CDR3 (Chothia) | QQYSKLPRT |
| 1323 | | |
| SEQ ID NO: | LC CDR1 (Combined) | SASQGISNYLN |
| 1321 | | |
| SEQ ID NO: | LC CDR2 (Combined) | YTSSLHS |
| 1322 | | |
| SEQ ID NO: | LC CDR3 (Combined) | QQYSKLPRT |
| 1323 | | |
| SEQ ID NO: | VH | DIQMTQTTSSLSASLGDRVTISCSASQGISNYLNWYQQKPD |
| 232 | | GTVKLLIYYTSSLHSGVPSRFSGSGSGTDYSLTISNLEPEDIA |
| | | TYYCQQYSKLPRTFGGGTKVEIK |
| SEQ ID NO: | VL | QVQLKESGPGLVAPSQSLSITCTVSGFSLTAYGVNWVRQPP |
| 233 | | GKGLEWLGMIWGDGNTDYNSALKSRLSISKDNSKSQVFLK |
| | | MNSLQTDDTARYYCARDRVTATLYAMDYWGQGTSVTVSS |

Humanized antibody C C-H-1 antibody

| | | |
|---|---|---|
| SEQ ID NO: | HC CDR1 (Kabat) | AYGVN |
| 1315 | | |
| SEQ ID NO: | HC CDR2 (Kabat) | MIWGDGNTDYNSALKS |
| 1316 | | |
| SEQ ID NO: | HC CDR3 (Kabat) | DRVTATLYAMDY |
| 1317 | | |
| SEQ ID NO: | HC CDR1 (Chothia) | GFSLTAY |
| 1318 | | |
| SEQ ID NO: | HC CDR2 (Chothia) | WGDGN |
| 1319 | | |
| SEQ ID NO: | HC CDR3 (Chothia) | DRVTATLYAMDY |
| 1317 | | |
| SEQ ID NO: | HC CDR1 (Combined) | GFSLTAYGVN |
| 1320 | | |
| SEQ ID NO: | HC CDR2 (Combined) | MIWGDGNTDYNSALKS |
| 1316 | | |
| SEQ ID NO: | HC CDR3 (Combined) | DRVTATLYAMDY |
| 1317 | | |
| SEQ ID NO: | LC CDR1 (Kabat) | SASQGISNYLN |
| 1321 | | |
| SEQ ID NO: | LC CDR2 (Kabat) | YTSSLHS |
| 1322 | | |
| SEQ ID NO: | LC CDR3 (Kabat) | QQYSKLPRT |
| 1323 | | |
| SEQ ID NO: | LC CDR1 (Chothia) | SASQGISNYLN |
| 1321 | | |
| SEQ ID NO: | LC CDR2 (Chothia) | YTSSLHS |
| 1322 | | |
| SEQ ID NO: | LC CDR3 (Chothia) | QQYSKLPRT |
| 1323 | | |
| SEQ ID NO: | LC CDR1 (Combined) | SASQGISNYLN |
| 1321 | | |
| SEQ ID NO: | LC CDR2 (Combined) | YTSSLHS |
| 1322 | | |
| SEQ ID NO: | LC CDR3 (Combined) | QQYSKLPRT |
| 1323 | | |
| SEQ ID NO: | VL | DIQMTQSPSSLSASVGDRVTITCSASQGISNYLNWYQQTPGK |
| 1324 | | APKLLIYYTSSLHSGVPSRFSGSGSGTDYTFTISSLQPEDIATY |
| | | YCQQYSKLPRTFGQGTKLQIT |
| SEQ ID NO: | VH | QVQLQESGPGLVRPSQTLSLTCTVSGFSLTAYGVNWVRQPP |
| 1325 | | GRGLEWLGMIWGDGNTDYNSALKSRVTMLKDTSKNQFSL |
| | | RLSSVTAADTAVYYCARDRVTATLYAMDYW |
| | | GQGSLVTVSS |

Humanized antibody C Variable light chain (VL)

| | | | |
|---|---|---|---|
| SEQ ID | VL | C-H-VL.1 | DIQMTQSPSFLSASVGDRVTITCSASQGISNYLNWYQQKPGKAVKLLIY |
| NO: 3000 | | | YTSSLHSGVPSRFSGSGSGTEYTLTISSLQPEDFATYYCQQYSKLPRTFGG |
| | | | GTKVEIK |
| SEQ ID | VL | C-H-VL.2 | DIQMTQSPSSLSASVGDRVTITCSASQGISNYLNWYQQKPGKAVKLLIY |
| NO: 3001 | | | YTSSLHSGVPSRFSGSGSGTDYTLTISSLQPEDFATYYCQQYSKLPRTFG |
| | | | GGTKVEIK |
| SEQ ID | VL | C-H-VL.3 | DIQMTQSPSSLSASVGDRVTITCSASQGISNYLNWYQQKPGKVVKLLIY |
| NO: 3002 | | | YTSSLHSGVPSRFSGSGSGTDYTLTISSLQPEDVATYYCQQYSKLPRTFG |
| | | | GGTKVEIK |
| SEQ ID | VL | C-H-VL.4 | DIQMTQSPSSLSASVGDRVTITCSASQGISNYLNWYQQKPGQAVKLLIY |
| NO: 3003 | | | YTSSLHSGVPSRFSGSGSGTDYTLTISSLQPEDVATYYCQQYSKLPRTFG |
| | | | GGTKVEIK |
| SEQ ID | VL | C-H-VL.5 | DIQMTQSPSSLSASVGDRVTITCSASQGISNYLNWYQQKPGKAVKLLIY |
| NO: 3004 | | | YTSSLHSGVPSRFSGSGSGTDYTFTISSLQPEDIATYYCQQYSKLPRTFGG |
| | | | GTKVEIK |
| SEQ ID | VL | C-H-VL.6 | DIQMTQSPSSLSASVGDRVTITCSASQGISNYLNWYQQKPGKTVKLLIYY |
| NO: 3005 | | | TSSLHSGIPSRFSGSGSGTDYTLTIRSLQPEDFATYYCQQYSKLPRTFGGG |
| | | | TKVEIK |
| SEQ ID | VL | C-H-VL.7 | AIQMTQSPSSLSASVGDRVTITCSASQGISNYLNWYQQKPGKAVKLLIY |
| NO: 3006 | | | YTSSLHSGVPSRFSGSGSGTDYTLTISSLQPEDFATYYCQQYSKLPRTFG |
| | | | GGTKVEIK |
| SEQ ID | VL | C-H-VL.8 | DIQMTQSPSSVSASVGDRVTITCSASQGISNYLNWYQQKPGKAVKLLIY |
| NO: 3007 | | | YTSSLHSGVPSRFSGSGSGTDYTLTISSLQPEDFATYYCQQYSKLPRTFG |
| | | | GGTKVEIK |
| SEQ ID | VL | C-H-VL.9 | DIQMTQSPSSLSASVGDRVTITCSASQGISNYLNWYQQKPGKAVKRLIY |
| NO: 3008 | | | YTSSLHSGVPSRFSGSGSGTEYTLTISNLQPEDFATYYCQQYSKLPRTFG |
| | | | GGTKVEIK |
| SEQ ID | VL | C-H- | AIRMTQSPFSLSASVGDRVTITCSASQGISNYLNWYQQKPAKAVKLFIYY |
| NO: 3009 | | VL.10 | TSSLHSGVPSRFSGSGSGTDYTLTISSLQPEDFATYYCQQYSKLPRTFGG |
| | | | GTKVEIK |
| SEQ ID | VL | C-H- | DIQMTQSPSSLSASVGDRVTITCSASQGISNYLNWYQQKPGKAVKRLIY |
| NO: 3010 | | VL.11 | YTSSLHSGVPSRFSGSGSGTEYTLTISSLQPEDFATYYCQQYSKLPRTFGG |
| | | | GTKVEIK |
| SEQ ID | VL | C-H- | DIQMTQSPSTLSASVGDRVTITCSASQGISNYLNWYQQKPGKAVKLLIY |
| NO: 3011 | | VL.12 | YTSSLHSGVPSRFSGSGSGTEYTLTISSLQPDDFATYYCQQYSKLPRTFG |
| | | | GGTKVEIK |
| SEQ ID | VL | C-H- | DIQMTQSPSSLSASVGDRVTITCSASQGISNYLNWYQQKPGKAVKSLIY |
| NO: 3012 | | VL.13 | YTSSLHSGVPSRFSGSGSGTDYTLTISSLQPEDFATYYCQQYSKLPRTFG |
| | | | GGTKVEIK |
| SEQ ID | VL | C-H- | DIQMTQSPSSLSASVGDRVTITCSASQGISNYLNWYQQKPGKAVKSLIY |
| NO: 3013 | | VL.14 | YTSSLHSGVPSKFSGSGSGTDYTLTISSLQPEDFATYYCQQYSKLPRTFG |
| | | | GGTKVEIK |
| SEQ ID | VL | C-H- | DIQMTQSPSSLSASVGDRVTITCSASQGISNYLNWYQQKPEKAVKSLIYY |
| NO: 3014 | | VL.15 | TSSLHSGVPSRFSGSGSGTDYTLTISSLQPEDFATYYCQQYSKLPRTFGG |
| | | | GTKVEIK |
| SEQ ID | VL | C-H- | DIQMTQSPSAMSASVGDRVTITCSASQGISNYLNWYQQKPGKVVKRLIY |
| NO: 3015 | | VL.16 | YTSSLHSGVPSRFSGSGSGTEYTLTISSLQPEDFATYYCQQYSKLPRTFGG |
| | | | GTKVEIK |
| SEQ ID | VL | C-H- | DIVMTQSPDSLAVSLGERATINCSASQGISNYLNWYQQKPGQPVKLLIY |
| NO: 3016 | | VL.17 | YTSSLHSGVPDRFSGSGSGTDYTLTISSLQAEDVAVYYCQQYSKLPRTFG |
| | | | GGTKVEIK |
| SEQ ID | VL | C-H- | EIVMTQSPGTLSLSPGERATLSCSASQGISNYLNWYQQKPGQAVKLLIY |
| NO: 3017 | | VL.18 | YTSSLHSGIPDRFSGSGSGTDYTLTISRLEPEDFAVYYCQQYSKLPRTFGG |
| | | | GTKVEIK |
| SEQ ID | VL | C-H- | EIVMTQSPPTLSLSPGERVTLSCSASQGISNYLNWYQQKPGQAVKLLIYY |
| NO: 3018 | | VL.19 | TSSLHSGIPARFSGSGSGTDYTLTISSLQPEDFAVYYCQQYSKLPRTFGGG |
| | | | TKVEIK |
| SEQ ID | VL | C-H- | EIVMTQSPPTLSLSPGERVTLSCSASQGISNYLNWYQQKPGQAVKLLIYY |
| NO: 3019 | | VL.20 | TSSLHSSIPARFSGSGSGTDYTLTISSLQPEDFAVYYCQQYSKLPRTFGGG |
| | | | TKVEIK |
| SEQ ID | VL | C-H- | EIVMTQSPATLSLSPGERATLSCSASQGISNYLNWYQQKPGQAVKLLIY |
| NO: 3020 | | VL.21 | YTSSLHSGIPARFSGSGSGTDYTLTISSLEPEDFAVYYCQQYSKLPRTFGG |
| | | | GTKVEIK |
| SEQ ID | VL | C-H- | EIVMTQSPATLSLSPGERATLSCSASQGISNYLNWYQQKPGQAVKLLIY |
| NO: 3021 | | VL.22 | YTSSLHSGIPARFSGSGSGTDYTLTISRLEPEDFAVYYCQQYSKLPRTFGG |
| | | | GTKVEIK |
| SEQ ID | VL | C-H- | EIVMTQSPATLSLSPGERATLSCSASQGISNYLNWYQQKPGQAVKLLIY |
| NO: 3022 | | VL.23 | YTSSLHSGIPDRFSGSGSGTDYTLTISRLEPEDFAVYYCQQYSKLPRTFGG |
| | | | GTKVEIK |
| SEQ ID | VL | C-H- | EIVMTQSPATLSLSPGERATLSCSASQGISNYLNWYQQKPGLAVKLLIYY |
| NO: 3023 | | VL.24 | TSSLHSGIPDRFSGSGSGTDYTLTISRLEPEDFAVYYCQQYSKLPRTFGGG |
| | | | TKVEIK |
| SEQ ID | VL | C-H- | DIQMIQSPSFLSASVGDRVSIICSASQGISNYLNWYLQKPGKSVKLFIYYT |
| NO: 3024 | | VL.25 | SSLHSGVSSRFSGRGSGTDYTLTIISLKPEDFAAYYCQQYSKLPRTFGGG |
| | | | TKVEIK |
| SEQ ID | VL | C-H- | EIVMTQSPATLSLSPGERATLSCSASQGISNYLNWYQQKPGQAVKLLIY |
| NO: 3025 | | VL.26 | YTSSLHSGIPARFSGSGSGTDYTLTISSLQPEDFAVYYCQQYSKLPRTFGG |
| | | | GTKVEIK |
| SEQ ID | VL | C-H- | EIVMTQSPATLSLSPGERATLSCSASQGISNYLNWYQQKPGQAVKLLIY |
| NO: 3026 | | VL.27 | YTSSLHSGIPARFSGSGPGTDYTLTISSLEPEDFAVYYCQQYSKLPRTFGG |
| | | | GTKVEIK |
| SEQ ID | VL | C-H- | DIVMTQTPLSLSVTPGQPASISCSASQGISNYLNWYLQKPGQSVKLLIYY |
| NO: 3027 | | VL.28 | TSSLHSGVPDRESGSGSGTDYTLKISRVEAEDVGVYYCQQYSKLPRTFG |
| | | | GGTKVEIK |
| SEQ ID | VL | C-H- | DIVMTQTPLSLSVTPGQPASISCSASQGISNYLNWYLQKPGQPVKLLIYY |
| NO: 3028 | | VL.29 | TSSLHSGVPDRFSGSGSGTDYTLKISRVEAEDVGVYYCQQYSKLPRTFG |
| | | | GGTKVEIK |
| SEQ ID | VL | C-H- | DIVMTQSPAFLSVTPGEKVTITCSASQGISNYLNWYQQKPDQAVKLLIY |
| NO: 3029 | | VL.30 | YTSSLHSGVPSRFSGSGSGTDYTFTISSLEAEDAATYYCQQYSKLPRTFG |
| | | | GGTKVEIK |
| SEQ ID | VL | C-H- | DIVMTQSPLSLPVTPGEPASISCSASQGISNYLNWYLQKPGQSVKLLIYY |
| NO: 3030 | | VL.31 | TSSLHSGVPDRFSGSGSGTDYTLKISRVEAEDVGVYYCQQYSKLPRTFG |
| | | | GGTKVEIK |
| SEQ ID | VL | C-H- | DIVMTQTPLSLPVTPGEPASISCSASQGISNYLNWYLQKPGQSVKLLIYY |
| NO: 3031 | | VL.32 | TSSLHSGVPDRFSGSGSGTDYTLKISRVEAEDVGVYYCQQYSKLPRTFG |
| | | | GGTKVEIK |
| SEQ ID | VL | C-H- | EIVMTQSPATLSVSPGERATLSCSASQGISNYLNWYQQKPGQAVKLLIY |
| NO: 3032 | | VL.33 | YTSSLHSGIPARFSGSGSGTEYTLTISILQSEDFAVYYCQQYSKLPRTFGG |
| | | | GTKVEIK |
| SEQ ID | VL | C-H- | EIVMTQSPATLSVSPGERATLSCSASQGISNYLNWYQQKPGQAVKLLIY |
| NO: 3033 | | VL.34 | YTSSLHSGIPARFSGSGSGTEYTLTISSLQSEDFAVYYCQQYSKLPRTFGG |
| | | | GTKVEIK |
| SEQ ID | VL | C-H- | DIVMTQSPLSLPVTLGQPASISCSASQGISNYLNWYQQRPGQSVKRLIYY |
| NO: 3034 | | VL.35 | TSSLHSGVPDRFSGSGSGTDYTLKISRVEAEDVGVYYCQQYSKLPRTFG |
| | | | GGTKVEIK |
| SEQ ID | VL | C-H- | EITMTQSPAFMSATPGDKVNISCSASQGISNYLNWYQQKPGEAVKFUIYY |
| NO: 3035 | | VL.36 | TSSLHSGIPPRESGSGYGTDYTLTINNIESEDAAYYYCQQYSKLPRTFGG |
| | | | GTKVEIK |
| SEQ ID | VL | C-H- | DIVMTQTPLSSPVTLGQPASISCSASQGISNYLNWYQQRPGQPVKLLIYY |
| NO: 3036 | | VL.37 | TSSLHSGVPDRFSGSGAGTDYTLKISRVEAEDVGVYYCQQYSKLPRTFG |
| | | | GGTKVEIK |
| SEQ ID | VL | C-H- | EIVMTQSPDFQSVTPKEKVTITCSASQGISNYLNWYQQKPDQSVKLLIYY |
| NO: 3037 | | VL.38 | TSSLHSGVPSRFSGSGSGTDYTLTINSLEAEDAATYYCQQYSKLPRTFGG |
| | | | GTKVEIK |
| SEQ ID | VL | C-H- | EIVMTQTPLSLSITPGEQASISCSASQGISNYLNWYLQKARPVVKLLIYYT |
| NO: 3038 | | VL.39 | SSLHSGVPDRFSGSGSGTDYTLKISRVEAEDFGVYYCQQYSKLPRTFGG |
| | | | GTKVEIK |
| SEQ ID | VL | C-H- | EIVMTQTPLSLSITPGEQASMSCSASQGISNYLNWYLQKARPVVKLLIYY |
| NO: 3039 | | VL.40 | TSSLHSGVPDRFSGSGSGTDYTLKISRVEAEDFGVYYCQQYSKLPRTFG |
| | | | GGTKVEIK |

Humanized antibody C Variable HEAVY chain (VH)

| | | | |
|---|---|---|---|
| SEQ ID | VH | C-H-VH.1 | QVTLKESGPVLVKPTETLTLTCTVSGFSLTAYGVNWVRQPPGKALEWL |
| NO: 3040 | | | GMIWGDGNTDYNSALKSRLTISKDNSKSQVVLTMTNMDPVDTATYYC |
| | | | ARDRVTATLYAMDYWGQGTLVTVSS |
| SEQ ID | VH | C-H-VH.2 | QVTLKESGPALVKPTETLTLTCTVSGFSLTAYGVNWVRQPPGKALEWL |
| NO: 3041 | | | GMIWGDGNTDYNSALKSRLIISKDNSKSQVVLTMTNMDPVDTATYYCA |
| | | | RDRVTATLYAMDYWGQGTLVTVSS |
| SEQ ID | VH | C-H-VH.3 | QVTLKESGPALVKPTQTLTLTCTVSGFSLTAYGVNWVRQPPGKALEWL |
| NO: 3042 | | | GMIWGDGNTDYNSALKSRLTISKDNSKSQVVLTMINMDPVDTATYYC |
| | | | ARDRVTATLYAMDYWGQGTLVTVSS |
| SEQ ID | VH | C-H-VH.4 | QVQLQESGPGLVKPSGTLSLTCAVSGFSLTAYGVNWVRQPPGKGLEWL |
| NO: 3043 | | | GMIWGDGNTDYNSALKSRLTISKDNSKSQVSLKLSSVTAADTAVYYCA |
| | | | RDRVTATLYAMDYWGQGTLVTVSS |
| SEQ ID | VH | C-H-VH.5 | QVTLKESGPTLVKPTQTLTLTCTVSGFSLTAYGVNWVRQPPGKALEWL |
| NO: 3044 | | | GMIWGDGNTDYNSALKSRLTITKDNSKSQVVLTMTNMDPVDTATYYC |
| | | | ARDRVTATLYAMDYWGQGTLVTVSS |
| SEQ ID | VH | C-H-VH.6 | QVTLKESGPALVKPTQTLTLTCTVSGFSLTAYGVNWVRQPPGKALEWL |
| NO: 3045 | | | GMIWGDGNTDYNSALKSRLTITKDNSKSQVVLTMTNMDPVDTATYYC |
| | | | ARDRVTATLYAMDYWGQGTLVTVSS |
| SEQ ID | VH | C-H-VH.7 | QVQLQESGPGLVKPSQTLSLTCTVSGFSLTAYGVNWVRQPPGKGLEWL |
| NO: 3046 | | | GMIWGDGNTDYNSALKSRLTISKDNSKSQVSLKLSSVTAADTAVYYCA |
| | | | RDRVTATLYAMDYWGQGTLVTVSS |
| SEQ ID | VH | C-H-VH.8 | QVQLQESGPGLVKPSETLSLTCTVSGFSLTAYGVNWVRQPPGKGLEWL |
| NO: 3047 | | | GMIWGDGNTDYNSALKSRLTISKDNSKSQVSLKLSSVTAADTAVYYCA |
| | | | RDRVTATLYAMDYWGQGTLVTVSS |
| SEQ ID | VH | C-H-VH.9 | QVQLQESGPGLVKPSQTLSLTCAVSGFSLTAYGVNWVRQPPGKGLEWL |
| NO: 3048 | | | GMIWGDGNTDYNSALKSRLTISKDNSKSQVSLKLSSVTAADTAVYYCA |
| | | | RDRVTATLYAMDYWGQGTLVTVSS |
| SEQ ID | VH | C-H- | QVQLQESGPGLVKPSDTLSLTCTVSGFSLTAYGVNWVRQPPGKGLEWL |
| NO: 3049 | | VH.10 | GMIWGDGNTDYNSALKSRLTISKDNSKSQVSLKLSSVTAADTAVYYCA |
| | | | RDRVTATLYAMDYWGQGTLVTVSS |
| SEQ ID | VH | C-H- | QVQLQESGPGLVKPSQTLSLTCTVSGFSLTAYGVNWVRQHPGKGLEWL |
| NO: 3050 | | VH.11 | GMIWGDGNTDYNSALKSRLTISKDNSKSQVSLKLSSVTAADTAVYYCA |
| | | | RDRVTATLYAMDYWGQGTLVTVSS |
| SEQ ID | VH | C-H- | QVQLQESGPGLVKPSQTLSLTCTVSGFSLTAYGVNWVRQPAGKGLEWL |
| NO: 3051 | | VH.12 | GMIWGDGNTDYNSALKSRLTISKDNSKSQVSLKLSSVTAADTAVYYCA |
| | | | RDRVTATLYAMDYWGQGTLVTVSS |
| SEQ ID | VH | C-H- | QVQLQESGPGLVKPSQTLSLTCAVSGFSLTAYGVNWVRQPPGKGLEWL |
| NO: 3052 | | VH.13 | GMIWGDGNTDYNSALKSRLTISKDNSKSQVSLKLSSVTAVDTAVYYCA |
| | | | RDRVTATLYAMDYWGQGTLVTVSS |
| SEQ ID | VH | C-H- | QVQLQESGPGLVKPSETLSLTCTVSGFSLTAYGVNWVRQPPGKGLEWL |
| NO: 3053 | | VH.14 | GMIWGDGNTDYNSALKSRLTISKDNSKSHVSLKLSSVTAADTAVYYCA |
| | | | RDRVTATLYAMDYWGQGTLVTVSS |
| SEQ ID | VH | C-H- | QVQLQESGPGLVKPSETLSLTCAVSGFSLTAYGVNWVRQPPGKGLEWL |
| NO: 3054 | | VH.15 | GMIWGDGNTDYNSALKSRLTISKDNSKSQVSLKLSSVTAADTAVYYCA |
| | | | RDRVTATLYAMDYWGQGTLVTVSS |
| SEQ ID | VH | C-H- | QVQLQESGPGLVKPSQTLSLTCAVYGFSLTAYGVNWVRQPPGKGLEWL |
| NO: 3055 | | VH.16 | GMIWGDGNTDYNSALKSRLTISKDNSKSQVSLKLSSVTAADTAVYYCA |
| | | | RDRVTATLYAMDYWGQGTLVTVSS |
| SEQ ID | VH | C-H- | RVQLQESGPGLVKPSETLSLTCTVSGFSLTAYGVNWVRQPPGKGLEWL |
| NO: 3056 | | VH.17 | GMIWGDGNTDYNSALKSRLTISKDNSKSQVPLKLSSVTAADTAVYYCA |
| | | | RDRVTATLYAMDYWGQGTLVTVSS |
| SEQ ID | VH | C-H- | QVQLQESGPGLVKPSQTLSLTCTVSGFSLTAYGVNWVRQHPGKGLEWL |
| NO: 3057 | | VH.18 | GMIWGDGNTDYNSALKSLLTISKDNSKSQVSLKLSSVTAADTAVYYCA |
| | | | RDRVTATLYAMDYWGQGTLVTVSS |
| SEQ ID | VH | C-H- | QVQLQESGPGLVKPSDTLSLTCAVSGFSLTAYGVNWVRQPPGKGLEWL |
| NO: 3058 | | VH.19 | GMIWGDGNTDYNSALKSRLTISKDNSKSQVSLKLSSVTALDTAVYYCA |
| | | | RDRVTATLYAMDYWGQGTLVTVSS |
| SEQ ID | VH | C-H- | QVQLQESGPGLVKPSDTLSLTCAVSGFSLTAYGVNWVRQPPGKGLEWL |
| NO: 3059 | | VH.20 | GMIWGDGNTDYNSALKSRLTISKDNSKSQVSLKLSSVTAVDTAVYYCA |
| | | | RDRVTATLYAMDYWGQGTLVTVSS |
| SEQ ID | VH | C-H- | QVQLQESGSGLVKPSQTLSLTCAVSGFSLTAYGVNWVRQPPGKGLEWL |
| NO: 3060 | | VH.21 | GMIWGDGNTDYNSALKSRLTISKDNSKSQVSLKLSSVTAADTAVYYCA |
| | | | RDRVTATLYAMDYWGQGTLVTVSS |
| SEQ ID | VH | C-H- | EVQLVESGGGLVQPGRSLRLSCTVSGFSLTAYGVNWVRQAPGKGLEWL |
| NO: 3061 | | VH.22 | GMIWGDGNTDYNSALKSRLTISKDNSKSIVYLQMNSLKTEDTAVYYCA |
| | | | RDRVTATLYAMDYWGQGTLVTVSS |
| SEQ ID | VH | C-H- | EVQLVESGGGLVQPGPSLRLSCTVSGFSLTAYGVNWVRQAPGKGLEWL |
| NO: 3062 | | VH.23 | GMIWGDGNTDYNSALKSRLTISKDNSKSIVYLQMNSLKTEDTAVYYCA |
| | | | RDRVTATLYAMDYWGQGTLVTVSS |
| SEQ ID | VH | C-H- | QVQLQESGSGLVKPSQTLSLTCAVSGFSLTAYGVNWVRQSPGKGLEWL |
| NO: 3063 | | VH.24 | GMIWGDGNTDYNSALKSRLTISKDNSKSQVSLKLSSVTAADTAVYYCA |
| | | | RDRVTATLYAMDYWGQGTLVTVSS |
| SEQ ID | VH | C-H- | QVQLQESGPGLVKPSETLSLTCTVSGFSLTAYGVNWVRQPAGKGLEWL |
| NO: 3064 | | VH.25 | GMIWGDGNTDYNSALKSRLTISKDNSKSQVSLKLSSVTAADTAVYYCA |
| | | | RDRVTATLYAMDYWGQGTLVTVSS |
| SEQ ID | VH | C-H- | EVQLVESGGGLVKPGRSLRLSCTVSGFSLTAYGVNWVRQAPGKGLEWL |
| NO: 3065 | | VH.26 | GMIWGDGNTDYNSALKSRLTISKDNSKSIVYLQMNSLKTEDTAVYYCA |
| | | | RDRVTATLYAMDYWGQGTLVTVSS |
| SEQ ID | VH | C-H- | QVQLQESGPGLVKPSETLSLTCAVYGFSLTAYGVNWVRQPPGKGLEWL |
| NO: 3066 | | VH.27 | GMIWGDGNTDYNSALKSRLTISKDNSKSQVYLKLSSVTAADTAVYYCA |
| | | | RDRVTATLYAMDYWGQGTLVTVSS |
| SEQ ID | VH | C-H- | QVQLQESGPGLVKPSDTLSLTCAVSGFSLTAYGVNWVRQPPGKGLEWL |
| NO: 3067 | | VH.28 | GMIWGDGNTDYNSALKSRLTISKDNSKSQVSLKLSSVTAVDTGVYYCA |
| | | | RDRVTATLYAMDYWGQGTLVTVSS |
| SEQ ID | VH | C-H- | EVQLVESGGGLVQPGGSLRLSCAVSGFSLTAYGVNWVRQAPGKGLEW |
| NO: 3068 | | VH.29 | LGMIWGDGNTDYNSALKSRLTISKDNSKSSVYLQMNSLKTEDTAVYYC |
| | | | ARDRVTATLYAMDYWGQGTLVTVSS |
| SEQ ID | VH | C-H- | EVQLVESGGGLVKPGGSLRLSCAVSGFSLTAYGVNWVRQAPGKGLEW |
| NO: 3069 | | VH.30 | LGMIWGDGNTDYNSALKSRLTISKDNSKSTVYLQMNSLKTEDTAVYYC |
| | | | ARDRVTATLYAMDYWGQGTLVTVSS |
| SEQ ID | VH | C-H- | QVQLQQSGPGLVKPSQTLSLTCAVSGFSLTAYGVNWVRQSPSRGLEWL |
| NO: 3070 | | VH.31 | GMIWGDGNTDYNSALKSRLTINKDNSKSQVSLQLNSVTPEDTAVYYCA |
| | | | RDRVTATLYAMDYWGQGTLVTVSS |
| SEQ ID | VH | C-H- | QVQLVESGGGLVQPGGSLRLSCSVSGFSLTAYGVNWVRQAPGKGLEW |
| NO: 3071 | | VH.32 | LGMIWGDGNTDYNSALKSRLTISKDNSKSTVYLQMNSLRAEDTAVYYC |
| | | | ARDRVTATLYAMDYWGQGTLVTVSS |
| SEQ ID | VH | C-H- | QVQLQQWGAGLLKPSETLSLTCAVYGFSLTAYGVNWVRQPPGKGLEW |
| NO: 3072 | | VH.33 | LGMIWGDGNTDYNSALKSRLTISKDNSKSQVSLKLSSVTAADTAVYYC |
| | | | ARDRVTATLYAMDYWGQGTLVTVSS |
| SEQ ID | VH | C-H- | QVQLVESGGGVVQPGRSLRLSCAVSGFSLTAYGVNWVRQAPGKGLEW |
| NO: 3073 | | VH.34 | LGMIWGDGNTDYNSALKSRLTISKDNSTSTVFLQMNSLRAEDTAVYYC |
| | | | ARDRVTATLYAMDYWGQGTLVTVSS |
| SEQ ID | VH | C-H- | EVQLVESGGGLVQPGGSLRLSCAVSGFSLTAYGVNWVRQAPGKGLEW |
| NO: 3074 | | VH.35 | LGMIWGDGNTDYNSALKSRLTISKDNSKSTVYLQMNSLRAEDTAVYYC |
| | | | ARDRVTATLYAMDYWGQGTLVTVSS |
| SEQ ID | VH | C-H- | EVQLVESGGGLVQPGGSLRLSCAVSGFSLTAYGVNWVRQAPGKGLEW |
| NO: 3075 | | VH.36 | LGMIWGDGNTDYNSALKSRLTISKDNAKSSVYLQMNSLRDEDTAVYYC |
| | | | ARDRVTATLYAMDYWGQGTLVTVSS |
| SEQ ID | VH | C-H- | EVQLLESGGGLVQPGGSLRLSCAVSGFSLTAYGVNWVRQAPGKGLEWL |
| NO: 3076 | | VH.37 | GMIWGDGNTDYNSALKSRLTISKDNSKSTVYLQMNSLRAEDTAVYYCA |
| | | | RDRVTATLYAMDYWGQGTLVTVSS |
| SEQ ID | VH | C-H- | QVQLVESGGGLVKPGGSLRLSCAVSGFSLTAYGVNWVRQAPGKGLEW |
| NO: 3077 | | VH.38 | LGMIWGDGNTDYNSALKSRLTISKDNAKSSVYLQMNSLRAEDTAVYYC |
| | | | ARDRVTATLYAMDYWGQGTLVTVSS |
| SEQ ID | VH | C-H- | EVQLVESGGGLVQPGGSLKLSCAVSGFSLTAYGVNWVRQASGKGLEW |
| NO: 3078 | | VH.39 | LGMIWGDGNTDYNSALKSRLTISKDNSKSTVYLQMNSLKTEDTAVYYC |
| | | | ARDRVTATLYAMDYWGQGTLVTVSS |
| SEQ ID | VH | C-H- | QVQLLESGGGLVKPGGSLRLSCAVSGFSLTAYGVNWVRQAPGKGLEW |
| NO: 3079 | | VH.40 | LGMIWGDGNTDYNSALKSRLTISKDNAKSSVYLQMNSLRAEDTAVYYC |
| | | | ARDRVTATLYAMDYWGQGTLVTVSS |
| SEQ ID | VH | C-H- | QVQLVESGGGVVQPGRSLRLSCAVSGFSLTAYGVNWVRQAPGKGLEW |
| NO: 3080 | | VH.41 | LGMIWGDGNTDYNSALKSRLTISKDNSKSTVYLQMNSLRAEDTAVYYC |
| | | | ARDRVTATLYAMDYWGQGTLVTVSS |
| SEQ ID | VH | C-H- | QVQLVESGGGVVQPGRSLRLSCAVSGFSLTAYGVNWVRQAPGKGLEW |
| NO: 3081 | | VH.42 | LGMIWGDGNTDYNSALKSRLTISKDNSKSRVYLQMNSLRAEDTAVYYC |
| | | | ARDRVTATLYAMDYWGQGTLVTVSS |
| SEQ ID | VH | C-H- | QVQLVESGGGVVQPGRSLRLSCAVSGFSLTAYGVNWVRQAPGKGLEW |
| NO: 3082 | | VH.43 | LGMIWGDGNTDYNSALKSRLAISKDNSKSTVYLQMNSLRAEDTAVYYC |
| | | | ARDRVTATLYAMDYWGQGTLVTVSS |
| SEQ ID | VH | C-H- | QVQLVESGGGVVQPGGSLRLSCAVSGFSLTAYGVNWVRQAPGKGLEW |
| NO: 3083 | | VH.44 | LGMIWGDGNTDYNSALKSRLTISKDNSKSTVYLQMNSLRAEDTAVYYC |
| | | | ARDRVTATLYAMDYWGQGTLVTVSS |
| SEQ ID | VH | C-H- | EVQLVESGGGLVQPGGSLRLSCAVSGFSLTAYGVNWVRQAPGKGLEW |
| NO: 3084 | | VH.45 | LGMIWGDGNTDYNSALKSRLTISKDNAKSTVYLQMNSLRAEDTAVYY |
| | | | CARDRVTATLYAMDYWGQGTLVTVSS |
| SEQ ID | VH | C-H- | EVQLVESGGGLVQPGGSLRLSCAVSGFSLTAYGVNWVRQAPGKGLEW |
| NO: 3085 | | VH.46 | LGMIWGDGNTDYNSALKSRLTISKDNAKSSVYLQMNSLRAEDTAVYYC |
| | | | ARDRVTATLYAMDYWGQGTLVTVSS |
| SEQ ID | VH | C-H- | EVQLVESGGVVVQPGGSLRLSCAVSGFSLTAYGVNWVRQAPGKGLEW |
| NO: 3086 | | VH.47 | LGMIWGDGNTDYNSALKSRLTISKDNSKSSVYLQMNSLRTEDTALYYC |
| | | | ARDRVTATLYAMDYWGQGTLVTVSS |
| SEQ ID | VH | C-H- | EVQLVESGGGLVQPGGSLRLSCAVSGFSLTAYGVNWVRQAPGKGLEW |
| NO: 3087 | | VH.48 | LGMIWGDGNTDYNSALKSRLTISKHNSKSTVYLQMNSLRAEDTAVYYC |
| | | | ARDRVTATLYAMDYWGQGTLVTVSS |
| SEQ ID | VH | C-H- | EVQLVESGGGLVKPGGSLRLSCAVSGFSLTAYGVNWVRQAPGKGLEW |
| NO: 3088 | | VH.49 | LGMIWGDGNTDYNSALKSRLTISKDNAKSSVYLQMNSLRAEDTAVYYC |
| | | | ARDRVTATLYAMDYWGQGTLVTVSS |
| SEQ ID | VH | C-H- | EVQLVESGGGLIQPGGSLRLSCAVSGFSLTAYGVNWVRQPPGKGLEWL |
| NO: 3089 | | VH.50 | GMIWGDGNTDYNSALKSRLTISKDNSKSTVYLQMNSLRAEDTAVYYCA |
| | | | RDRVTATLYAMDYWGQGTLVTVSS |

TABLE 11
Amino acid sequences for anti TCRβ V5 antibodies. Amino acid and nucleotide
sequences for murine and humanized antibody molecules which bind to TCRVB 5
(e.g., TCRVB 5-5 or TCRVB 5-6). The amino acid the heavy and light chain CDRs,
and the amino acid and nucleotide sequences of the heavy and light chain variable
regions, and the heavy and light chains are shown.
Murine antibody E, also referred to as MH3-2

| | | |
|---|---|---|
| SEQ ID NO: | HC CDR1 (Kabat) | SSWMN |
| 1298 | | |
| SEQ ID NO: | HC CDR2 (Kabat) | RIYPGDGDTKYNGKFKG |
| 1299 | | |
| SEQ ID NO: | HC CDR3 (Kabat) | RGTGGWYFDV |
| 1300 | | |
| SEQ ID NO: | HC CDR1 (Chothia) | GYAFSSS |
| 1302 | | |
| SEQ ID NO: | HC CDR2 (Chothia) | YPGDGD |
| 1303 | | |
| SEQ ID NO: | HC CDR3 (Chothia) | RGTGGWYFDV |
| 1301 | | |
| SEQ ID NO: | HC CDR1 (Combined) | GYAFSSSWMN |
| 1304 | | |
| SEQ ID NO: | HC CDR2 (Combined) | RIYPGDGDTKYNGKFKG |
| 1299 | | |
| SEQ ID NO: | HC CDR3 (Combined) | RGTGGWYFDV |
| 1301 | | |
| SEQ ID NO: | LC CDR1 (Kabat) | RASESVDSSGNSFMH |
| 1305 | | |
| SEQ ID NO: | LC CDR2 (Kabat) | RASNLES |
| 1306 | | |
| SEQ ID NO: | LC CDR3 (Kabat) | QQSFDDPFT |
| 1307 | | |
| SEQ ID NO: | LC CDR1 (Chothia) | SESVDSSGNSF |
| 1308 | | |
| SEQ ID NO: | LC CDR2 (Chothia) | RASNLES |
| 1306 | | |
| SEQ ID NO: | LC CDR3 (Chothia) | QQSFDDPFT |
| 1307 | | |
| SEQ ID NO: | LC CDR1 (Combined) | RASESVDSSGNSFMH |
| 1305 | | |
| SEQ ID NO: | LC CDR2 (Combined) | RASNLES |
| 1306 | | |
| SEQ ID NO: | LC CDR3 (Combined) | QQSFDDPFT |
| 1307 | | |
| SEQ ID NO: | VH | QVQLQQSGPELVKPGASVKISCKASGYAFSSSWMNWVKQR |
| 3091 | | PGQGLEWIGRIYPGDGDTKYNGKFKGKATLTADKSSSTAY |
| | | MHLSSLTSVDSAVYFCARRGTGGWYFDVWGAGTTVTVSS |
| SEQ ID NO: | Heavy chain | METDTLLLWVLLLWVPGSTGQVQLQQSGPELVKPGASVKI |
| 3284 | | SCKASGYAFSSSWMNWVKQRPGQGLEWIGRIYPGDGDTK |
| | | YNGKFKGKATLTADKSSSTAYMHLSSLTSVDSAVYFCARR |
| | | GTGGWYFDVWGAGTTVTVSSAKTTAPSVYPLAPVCGDTT |
| | | GSSVTLGCLVKGYFPEPVTLTWNSGSLSSGVHTFPAVLQSD |
| | | LYTLSSSVTVTSSTWPSQSITCNVAHPASSTKVDKKIEPRGP |
| | | TIKPCPPCKCPAPNLLGGPSVFIFPPKIKDVLMISLSPIVTCVV |
| | | VDVSEDDPDVQISWFVNNVEVHTAQTQTHREDYNSTLRVV |
| | | SALPIQHQDWMSGKEFKCKVNNKDLPAPIERTISKPKGSVR |
| | | APQVYVLPPPEEEMTKKQVTLTCMVTDFMPEDIYVEWTNN |
| | | GKTELNYKNTEPVLDSDGSYFMYSKLRVEKKNWVERNSYS |
| | | CSVVHEGLHNHHTTKSFSRTPGK |
| SEQ ID NO: | VL | DIVLTQSPASLAVSLGQRATISCRASESVDSSGNSFMHWYQ |
| 3092 | | QKPGQPPQLLIYRASNLESGIPARFSGSGSRTDFTLTINPVEA |
| | | DDVATFYCQQSFDDPFTFGSGTKLEIK |
| SEQ ID NO: | Light chain | METDTLLLWVLLLWVPGSTGDIVLTQSPASLAVSLGQRATI |
| 3285 | | SCRASESVDSSGNSFMHWYQQKPGQPPQLLIYRASNLESGIP |
| | | ARFSGSGSRTDFTLTINPVEADDVATFYCQQSFDDPFTFGSG |
| | | TKLEIKRADAAPTVSIFPPSSEQLTSGGASVVCFLNNFYPKDI |
| | | NVKWKIDGSERQNGVLNSWTDQDSKDSTYSMSSTLTLTKD |
| | | EYERHNSYTCEATHKTSTSPIVKSFNRNEC |

Humanized antibody E (E-H antibody)
Variable light chain (VL)

| | | | |
|---|---|---|---|
| SEQ ID | VL | E-H.1 | DIVLTQSPDSLAVSLGERATINCRASESVDSSGNSFMHWYQQKPGQPPQLLI |
| NO: 3093 | | | YRASNLESGVPDRFSGSGSRTDFTLTISSLQAEDVAVYYCQQSFDDPFTFGQ |
| | | | GTKLEIK |
| SEQ ID | VL | E-H.2 | EIVLTQSPATLSLSPGERATLSCRASESVDSSGNSFMHWYQQKPGQAPQLLI |
| NO: 3094 | | | YRASNLESGIPARFSGSGSRTDFTLTISSLEPEDFAVYYCQQSFDDPFTFGQGT |
| | | | KLEIK |
| SEQ ID | VL | E-H.3 | EIVLTQSPATLSLSPGERATLSCRASESVDSSGNSFMHWYQQKPGQAPQLLI |
| NO: 3095 | | | YRASNLESGIPARFSGSGSRTDFTLTISRLEPEDFAVYYCQQSFDDPFTFGQG |
| | | | TKLEIK |
| SEQ ID | VL | E-H.4 | EIVLTQSPATLSLSPGERATLSCRASESVDSSGNSFMHWYQQKPGQAPQLLI |
| NO: 3096 | | | YRASNLESGIPARFSGSGSRTDFTLTISSLQPEDFAVYYCQQSFDDPFTFGQG |
| | | | TKLEIK |
| SEQ ID | VL | E-H.5 | DIQLTQSPSSLSASVGDRVTITCRASESVDSSGNSFMHWYQQKPGQAPQLLI |
| NO: 3097 | | | YRASNLESGVPSRFSGSGSRTDFILTISSLQPEDVATYYCQQSFDDPFTFGQG |
| | | | TKLEIK |
| SEQ ID | VL | E-H.6 | EIVLTQSPATLSLSPGERATLSCRASESVDSSGNSFMHWYQQKPGQAPQLLI |
| NO: 3098 | | | YRASNLESGIPARFSGSGPRTDFTLTISSLEPEDFAVYYCQQSFDDPFTFGQGT |
| | | | KLEIK |
| SEQ ID | VL | E-H.7 | EIVLTQSPATLSLSPGERATLSCRASESVDSSGNSFMHWYQQKPGQAPQLLI |
| NO: 3099 | | | YRASNLESGIPDRFSGSGSRTDFTLTISRLEPEDFAVYYCQQSFDDPFTFGQG |
| | | | TKLEIK |
| SEQ ID | VL | E-H.8 | DIQLTQSPSSLSASVGDRVTITCRASESVDSSGNSFMHWYQQKPGKVPQLLI |
| NO: 3100 | | | YRASNLESGVPSRFSGSGSRTDFTLTISSLQPEDVATYYCQQSFDDPFTFGQG |
| | | | TKLEIK |
| SEQ ID | VL | E-H.9 | DIQLTQSPSSLSASVGDRVTITCRASESVDSSGNSFMHWYQQKPGKTPQLLIY |
| NO: 3101 | | | RASNLESGIPSRFSGSGSRTDFTLTIRSLQPEDFATYYCQQSFDDPFIFGQGTK |
| | | | LEIK |
| SEQ ID | VL | E- | EIVLTQSPGTLSLSPGERATLSCRASESVDSSGNSFMHWYQQKPGQAPQLLI |
| NO: 3102 | | H.10 | YRASNLESGIPDRFSGSGSRTDFTLTISRLEPEDFAVYYCQQSFDDPFTFGQG |
| | | | TKLEIK |
| SEQ ID | VL | E- | EIVLTQSPATLSLSPGERATLSCRASESVDSSGNSFMHWYQQKPGLAPQLLIY |
| NO: 3103 | | H.11 | RASNLESGIPDRFSGSGSRTDFTLTISRLEPEDFAVYYCQQSFDDPFTFGQGT |
| | | | KLEIK |
| SEQ ID | VL | E- | DIQLTQSPSSLSASVGDRVTITCRASESVDSSGNSFMHWYQQKPGKAPQLLI |
| NO: 3104 | | H.12 | YRASNLESGVPSRFSGSGSRTDFTLTISSLQPEDFATYYCQQSFDDPFTFGQG |
| | | | TKLEIK |
| SEQ ID | VL | E- | DIQLTQSPSSVSASVGDRVTITCRASESVDSSGNSFMHWYQQKPGKAPQLLI |
| NO: 3105 | | H.13 | YRASNLESGVPSRFSGSGSRTDFTLTISSLQPEDFATYYCQQSFDDPFTFGQG |
| | | | TKLEIK |
| SEQ ID | VL | E- | AIQLTQSPSSLSASVGDRVTITCRASESVDSSGNSFMHWYQQKPGKAPQLLI |
| NO: 3106 | | H.14 | YRASNLESGVPSRFSGSGSRTDFTLTISSLQPEDFATYYCQQSFDDPFTFGQG |
| | | | TKLEIK |
| SEQ ID | VL | E- | DIQLTQSPSFLSASVGDRVTITCRASESVDSSGNSFMHWYQQKPGKAPQLLI |
| NO: 3107 | | H.15 | YRASNLESGVPSRFSGSGSRTEFTLTISSLQPEDFATYYCQQSFDDPFTFGQG |
| | | | TKLEIK |
| SEQ ID | VL | E- | DIQLTQSPSSLSASVGDRVTITCRASESVDSSGNSFMHWYQQKPGKAPQLLI |
| NO: 3108 | | H.16 | YRASNLESGVPSRFSGSGSRTDFTFTISSLQPEDIATYYCQQSFDDPFTFGQGT |
| | | | KLEIK |
| SEQ ID | VL | E- | EIVLTQSPATLSVSPGERATLSCRASESVDSSGNSFMHWYQQKPGQAPQLLI |
| NO: 3109 | | H.17 | YRASNLESGIPARFSGSGSRTEFTLTISILQSEDFAVYYCQQSFDDPFTFGQGT |
| | | | KLEIK |
| SEQ ID | VL | E- | EIVLTQSPATLSVSPGERATLSCRASESVDSSGNSFMHWYQQKPGQAPQLLI |
| NO: 3110 | | H.18 | YRASNLESGIPARFSGSGSRTEFTLTISSLQSEDFAVYYCQQSFDDPFTFGQGT |
| | | | KLEIK |
| SEQ ID | VL | E- | AIRLTQSPFSLSASVGDRVTITCRASESVDSSGNSFMHWYQQKPAKAPQLFIY |
| NO: 3111 | | H.19 | RASNLESGVPSRFSGSGSRTDFTLTISSLQPEDFATYYCQQSFDDPFIFGQGT |
| | | | KLEIK |
| SEQ ID | VL | E- | DIQLTQSPSSLSASVGDRVTITCRASESVDSSGNSFMHWYQQKPGKAPQSLI |
| NO: 3112 | | H.20 | YRASNLESGVPSRFSGSGSRTDFTLTISSLQPEDFATYYCQQSFDDPFTFGQG |
| | | | TKLEIK |
| SEQ ID | VL | E- | DIQLTQSPSSLSASVGDRVTITCRASESVDSSGNSFMHWYQQKPGKAPQRLI |
| NO: 3113 | | H.21 | YRASNLESGVPSRFSGSGSRTEFTLTISNLQPEDFATYYCQQSFDDPFTFGQG |
| | | | TKLEIK |
| SEQ ID | VL | E- | DIQLTQSPSTLSASVGDRVTITCRASESVDSSGNSFMHWYQQKPGKAPQLLI |
| NO: 3114 | | H.22 | YRASNLESGVPSRFSGSGSRTEFTLTISSLQPDDFATYYCQQSFDDPFTFGQG |
| | | | TKLEIK |
| SEQ ID | VL | E- | EIVLTQSPDFQSVTPKEKVTITCRASESVDSSGNSFMHWYQQKPDQSPQLLIY |
| NO: 3115 | | H.23 | RASNLESGVPSRFSGSGSRTDFTLTINSLEAEDAATYYCQQSFDDPFTFGQGT |
| | | | KLEIK |
| SEQ ID | VL | E- | DIQLTQSPSSLSASVGDRVTITCRASESVDSSGNSFMHWYQQKPGKAPQSLI |
| NO: 3116 | | H.24 | YRASNLESGVPSKFSGSGSRTDFTLTISSLQPEDFATYYCQQSFDDPFTFGQG |
| | | | TKLEIK |
| SEQ ID | VL | E- | DIQLTQSPSSLSASVGDRVTITCRASESVDSSGNSFMHWYQQKPGKAPQRLI |
| NO: 3117 | | H.25 | YRASNLESGVPSRFSGSGSRTEFTLTISSLQPEDFATYYCQQSFDDPFTFGQG |
| | | | TKLEIK |
| SEQ ID | VL | E- | DIVLTQTPLSLSVTPGQPASISCRASESVDSSGNSFMHWYLQKPGQPPQLLIY |
| NO: 3118 | | H.26 | RASNLESGVPDRESGSGSRTDFTLKISRVEAEDVGVYYCQQSFDDPFTFGQG |
| | | | TKLEIK |
| SEQ ID | VL | E- | DIQLTQSPSSLSASVGDRVTITCRASESVDSSGNSFMHWYQQKPEKAPQSLIY |
| NO: 3119 | | H.27 | RASNLESGVPSRFSGSGSRTDFTLTISSLQPEDFATYYCQQSFDDPFTFGQGT |
| | | | KLEIK |
| SEQ ID | VL | E- | EIVLTQSPPTLSLSPGERVTLSCRASESVDSSGNSFMHWYQQKPGQAPQLLIY |
| NO: 3120 | | H.28 | RASNLESGIPARFSGSGSRTDFTLTISSLQPEDFAVYYCQQSFDDPFTFGQGT |
| | | | KLEIK |
| SEQ ID | VL | E- | DIQLTQSPSAMSASVGDRVTITCRASESVDSSGNSFMHWYQQKPGKVPQRLI |
| NO: 3121 | | H.29 | YRASNLESGVPSRFSGSGSRTEFTLTISSLQPEDFATYYCQQSFDDPFTFGQG |
| | | | TKLEIK |
| SEQ ID | VL | E- | DIVLTQSPLSLPVTPGEPASISCRASESVDSSGNSFMHWYLQKPGQSPQLLIY |
| NO: 3122 | | H.30 | RASNLESGVPDRFSGSGSRTDFTLKISRVEAEDVGVYYCQQSFDDPFTFGQG |
| | | | TKLEIK |
| SEQ ID | VL | E- | DIVLTQTPLSLPVTPGEPASISCRASESVDSSGNSFMHWYLQKPGQSPQLLIY |
| NO: 3123 | | H.31 | RASNLESGVPDRFSGSGSRTDFTLKISRVEAEDVGVYYCQQSFDDPFTFGQG |
| | | | TKLEIK |
| SEQ ID | VL | E- | DIVLTQTPLSLSVTPGQPASISCRASESVDSSGNSFMHWYLQKPGQSPQLLIY |
| NO: 3124 | | H.32 | RASNLESGVPDRFSGSGSRTDFTLKISRVEAEDVGVYYCQQSFDDPFTFGQG |
| | | | TKLEIK |
| SEQ ID | VL | E- | EIVLTQSPPTLSLSPGERVTLSCRASESVDSSGNSFMHWYQQKPGQAPQLLIY |
| NO: 3125 | | H.33 | RASNLESSIPARFSGSGSRTDFTLTISSLQPEDFAVYYCQQSFDDPFTFGQGTK |
| | | | LEIK |
| SEQ ID | VL | E- | DIVLTQSPLSLPVTLGQPASISCRASESVDSSGNSFMHWYQQRPGQSPQRLIY |
| NO: 3126 | | H.34 | RASNLESGVPDRFSGSGSRTDFTLKISRVEAEDVGVYYCQQSFDDPFTFGQG |
| | | | TKLEIK |
| SEQ ID | VL | E- | DIVLTQTPLSSPVTLGQPASISCRASESVDSSGNSFMHWYQQRPGQPPQLLIY |
| NO: 3127 | | H.35 | RASNLESGVPDRFSGSGARTDFTLKISRVEAEDVGVYYCQQSFDDPFTFGQG |
| | | | TKLEIK |
| SEQ ID | VL | E- | DIVLTQSPAFLSVTPGEKVTITCRASESVDSSGNSFMHWYQQKPDQAPQLLI |
| NO: 3128 | | H.36 | YRASNLESGVPSRFSGSGSRTDFTFTISSLEAEDAATYYCQQSFDDPFTFGQG |
| | | | TKLEIK |
| SEQ ID | VL | H.37 | DIQLIQSPSFLSASVGDRVSIICRASESVDSSGNSFMHWYLQKPGKSPQLFIYR |
| NO: 3129 | | | ASNLESGVSSRFSGRGSRTDFTLTIISLKPEDFAAYYCQQSFDDPFTFGQGTK |
| | | | LEIK |
| SEQ ID | VL | E- | EIVLTQTPLSLSITPGEQASISCRASESVDSSGNSFMHWYLQKARPVPQLLIYR |
| NO: 3130 | | H.38 | ASNLESGVPDRFSGSGSRTDFTLKISRVEAEDFGVYYCQQSFDDPFTFGQGT |
| | | | KLEIK |
| SEQ ID | VL | E- | EIVLTQTPLSLSITPGEQASMSCRASESVDSSGNSFMHWYLQKARPVPQLLIY |
| NO: 3131 | | H.39 | RASNLESGVPDRFSGSGSRTDFTLKISRVEAEDFGVYYCQQSFDDPFTFGQG |
| | | | TKLEIK |
| SEQ ID | VL | E- | EITLTQSPAFMSATPGDKVNISCRASESVDSSGNSFMHWYQQKPGEAPQFLY |
| NO: 3132 | | H.40 | RASNLESGIPPRFSGSGYRTDFTLTINNIESEDAAYYYCQQSFDDPFTFGQGT |
| | | | KLEIK |

Variable HEAVY chain (VH)

| | | | |
|---|---|---|---|
| SEQ ID | VH | E-H.1 | QVQLVQSGAEVKKPGASVKVSCKASGYAFSSSWMNWVRQAPGQGLEWIG |
| NO: 3133 | | | RIYPGDGDTKYNGKFKGRATLTADKSTSTAYMELSSLRSEDTAVYYCARRG |
| | | | TGGWYFDVWGQGTTVTVSS |
| SEQ ID | VH | E-H.2 | QVQLVQSGAEVKKPGSSVKVSCKASGYAFSSSWMNWVRQAPGQGLEWIG |
| NO: 3134 | | | RIYPGDGDTKYNGKFKGRATLTADKSTSTAYMELSSLRSEDTAVYYCARRG |
| | | | TGGWYFDVWGQGTTVTVSS |
| SEQ ID | VH | E-H.3 | QVQLVQSGAEVKKPGASVKVSCKASGYAFSSSWMNWVRQAPGKGLEWIG |
| NO: 3135 | | | RIYPGDGDTKYNGKFKGRATLTADKSTSTAYMELSSLRSEDTAVYYCARRG |
| | | | TGGWYFDVWGQGTTVTVSS |
| SEQ ID | VH | E-H.4 | QVQLVQSGAEVKKPGASVKVSCKASGYAFSSSWMNWVRQAPGQELEWIG |
| NO: 3136 | | | RIYPGDGDIKYNGKFKGRATLTADKSISTAYMELSSLRSEDTATYYCARRG |
| | | | TGGWYFDVWGQGTTVTVSS |
| SEQ ID | VH | E-H.5 | EVQLVQSGAEVKKPGATVKISCKASGYAFSSSWMNWVQQAPGKGLEWIGR |
| NO: 3137 | | | TYPGDGDTKYNGKFKGRATLTADKSTSTAYMELSSLRSEDTAVYYCARRGT |
| | | | GGWYFDVWGQGTTVTVSS |
| SEQ ID | VH | E-H.6 | QVQLVQSGAEVKKTGSSVKVSCKASGYAFSSSWMNWVRQAPGQALEWIG |
| NO: 3138 | | | RIYPGDGDTKYNGKFKGRATLTADKSMSTAYMELSSLRSEDTAMYYCARR |
| | | | GTGGWYFDVWGQGTTVTVSS |
| SEQ ID | VH | E-H.7 | QVQLVQSGAEVKKPGASVKVSCKASGYAFSSSWMNWVRQAPGQRLEWIG |
| NO: 3139 | | | RIYPGDGDTKYNGKFKGRATLTADKSASTAYMELSSLRSEDMAVYYCARR |
| | | | GTGGWYFDVWGQGTTVTVSS |
| SEQ ID | VH | E-H.8 | QVQLVQSGAEVKKPGASVKVSCKASGYAFSSSWMNWVRQAPGQGLEWIG |
| NO: 3140 | | | RIYPGDGDTKYNGKFKGRATLTADKSTSTAYMELRSLRSDDMAVYYCARR |
| | | | GTGGWYFDVWGQGTTVTVSS |
| SEQ ID | VH | E-H.9 | QVQLVQSGAEVKKPGASVKVSCKASGYAFSSSWMNWVRQAPGQRLEWIG |
| NO: 3141 | | | RIYPGDGDTKYNGKFKGRATLTADKSASTAYMELSSLRSEDTAVYYCARRG |
| | | | TGGWYFDVWGQGTTVTVSS |
| SEQ ID | VH | E- | QVQLVQSGAEVKKPGASVKVSCKASGYAFSSSWMNWVRQAPGQGLEWIG |
| NO: 3142 | | H.10 | RIYPGDGDTKYNGKFKGRATLTADKSTSTAYMELRSLRSDDTAVYYCARR |
| | | | GTGGWYFDVWGQGTTVTVSS |
| SEQ ID | VH | E- | QVQLVQSGAEVKKPGASVKVSCKASGYAFSSSWMNWVRQAPGQGLEWIG |
| NO: 3143 | | H.11 | RIYPGDGDTKYNGKFKGRATLTADKSISTAYMELSRLRSDDTAVYYCARRG |
| | | | TGGWYFDVWGQGTTVTVSS |
| SEQ ID | VH | E- | QVQLVQSGAEVKKPGASVKVSCKASGYAFSSSWMNWVRQAPGQGLEWIG |
| NO: 3144 | | H.12 | RIYPGDGDTKYNGKFKGRATLTADKSISTAYMELSRLRSDDTVVYYCARRG |
| | | | TGGWYFDVWGQGTTVTVSS |
| SEQ ID | VH | E- | QVQLVQSGAEVKKPGASVKVSCKASGYAFSSSWMNWVRQAPGQGLEWIG |
| NO: 3145 | | H.13 | RIYPGDGDTKYNGKFKGWATLTADKSISTAYMELSRLRSDDTAVYYCARR |
| | | | GTGGWYFDVWGQGTTVTVSS |
| SEQ ID | VH | E- | QVQLVQSGAEVKKPGASVKVSCKASGYAFSSSWMNWVRQATGQGLEWIG |
| NO: 3146 | | H.14 | RIYPGDGDTKYNGKFKGRATLTANKSISTAYMELSSLRSEDTAVYYCARRG |
| | | | TGGWYFDVWGQGTTVTVSS |
| SEQ ID | VH | E- | QVQLVQSGSELKKPGASVKVSCKASGYAFSSSWMNWVRQAPGQGLEWIGR |
| NO: 3147 | | H.15 | IYPGDGDTKYNGKFKGRAVLSADKSVSTAYLQISSLKAEDTAVYYCARRGT |
| | | | GGWYFDVWGQGTTVTVSS |
| SEQ ID | VH | E- | QVQLVQSGPEVKKPGTSVKVSCKASGYAFSSSWMNWVRQARGQRLEWIG |
| NO: 3148 | | H.16 | RIYPGDGDTKYNGKFKGRATLTADKSTSTAYMELSSLRSEDTAVYYCARRG |
| | | | TGGWYFDVWGQGTTVTVSS |
| SEQ ID | VH | E- | EVQLVQSGAEVKKPGESLKISCKASGYAFSSSWMNWVRQMPGKGLEWIGR |
| NO: 3149 | | H.17 | IYPGDGDTKYNGKFKGQATLSADKSISTAYLQWSSLKASDTAMYYCARRG |
| | | | TGGWYFDVWGQGTTVTVSS |
| SEQ ID | VH | E- | QVQLVQSGSELKKPGASVKVSCKASGYAFSSSWMNWVRQAPGQGLEWIGR |
| NO: 3150 | | H.18 | IYPGDGDTKYNGKFKGRAVLSADKSVSMAYLQISSLKAEDTAVYYCARRG |
| | | | TGGWYFDVWGQGTTVTVSS |
| SEQ ID | VH | E- | QVQLVQSGHEVKQPGASVKVSCKASGYAFSSSWMNWVPQAPGQGLEWIG |
| NO: 3151 | | H.19 | RIYPGDGDTKYNGKFKGRAVLSADKSASTAYLQISSLKAEDMAMYYCARR |
| | | | GTGGWYFDVWGQGTTVTVSS |
| SEQ ID | VH | E- | EVQLVQSGAEVKKPGESLKISCKASGYAFSSSWMNWVRQMPGKGLEWIGR |
| NO: 3152 | | H.20 | IYPGDGDTKYNGKFKGQATLSADKPISTAYLQWSSLKASDTAMYYCARRG |
| | | | TGGWYFDVWGQGTTVTVSS |
| SEQ ID | VH | E- | EVQLVQSGAEVKKPGESLRISCKASGYAFSSSWMNWVRQMPGKGLEWIGRI |
| NO: 3153 | | H.21 | YPGDGDTKYNGKFKGQATLSADKSISTAYLQWSSLKASDTAMYYCARRGT |
| | | | GGWYFDVWGQGTTVTVSS |
| SEQ ID | VH | E- | EVQLVQSGAEVKKPGESLRISCKASGYAFSSSWMNWVRQMPGKGLEWIGRI |
| NO: 3154 | | H.22 | YPGDGDTKYNGKFKGHATLSADKSISTAYLQWSSLKASDTAMYYCARRGT |
| | | | GGWYFDVWGQGTTVTVSS |
| SEQ ID | VH | E- | QVQLVQSGAEVKKTGSSVKVSCKASGYAFSSSWMNWVRQAPRQALEWIG |
| NO: 3155 | | H.23 | RIYPGDGDTKYNGKFKGRATLTADKSMSTAYMELSSLRSEDTAMYYCARR |
| | | | GTGGWYFDVWGQGTTVTVSS |
| SEQ ID | VH | E- | EVQLVESGGGLVQPGRSLRLSCTASGYAFSSSWMNWVRQAPGKGLEWIGRI |
| NO: 3156 | | H.24 | YPGDGDTKYNGKFKGRATLSADKSKSIAYLQMNSLKTEDTAVYYCARRGT |
| | | | GGWYFDVWGQGTTVTVSS |
| SEQ ID | VH | E- | EVQLVESGGGLVQPGPSLRLSCTASGYAFSSSWMNWVRQAPGKGLEWIGRI |
| NO: 3157 | | H.25 | YPGDGDTKYNGKFKGRATLSADKSKSIAYLQMNSLKTEDTAVYYCARRGT |
| | | | GGWYFDVWGQGTTVTVSS |
| SEQ ID | VH | E- | QVQLQESGPGLVKPSQTLSLTCTASGYAFSSSWMNWVRQPPGKGLEWIGRI |
| NO: 3158 | | H.26 | YPGDGDTKYNGKFKGRATLSADKSKSQASLKLSSVTAADTAVYYCARRGT |
| | | | GGWYFDVWGQGTTVTVSS |
| SEQ ID | VH | E- | QVQLQESGPGLVKPSGTLSLTCAASGYAFSSSWMNWVRQPPGKGLEWIGRI |
| NO: 3159 | | H.27 | YPGDGDTKYNGKFKGRATLSADKSKSQASLKLSSVTAADTAVYYCARRGT |
| | | | GGWYFDVWGQGTTVTVSS |
| SEQ ID | VH | E- | EVQLVESGGGLVKPGRSLRLSCTASGYAFSSSWMNWVRQAPGKGLEWIGRI |
| NO: 3160 | | H.28 | YPGDGDTKYNGKFKGRATISADKSKSIAYLQMNSLKTEDTAVYYCARRGT |
| | | | GGWYFDVWGQGTTVTVSS |
| SEQ ID | VH | E- | EVQLVESGGGLVQPGGSLKLSCAASGYAFSSSWMNWVRQASGKGLEWIGR |
| NO: 3161 | | H.29 | IYPGDGDTKYNGKFKGRAILSADKSKSTAYLQMNSLKTEDTAVYYCARRG |
| | | | TGGWYFDVWGQGTTVTVSS |
| SEQ ID | VH | E- | QVQLQESGPGLVKPSQTLSLTCAASGYAFSSSWMNWVRQPPGKGLEWIGRI |
| NO: 3162 | | H.30 | YPGDGDTKYNGKFKGRATLSADKSKSQASLKLSSVTAADTAVYYCARRGT |
| | | | GGWYFDVWGQGTTVTVSS |
| SEQ ID | VH | E- | EVQLVESGGGLVKPGGSLRLSCAASGYAFSSSWMNWVRQAPGKGLEWIGR |
| NO: 3163 | | H.31 | IYPGDGDTKYNGKFKGRATLSADKSKSTAYLQMNSLKTEDTAVYYCARRG |
| | | | TGGWYFDVWGQGTTVTVSS |
| SEQ ID | VH | E- | EVQLVESGGALVKPGGSLRLSCAASGYAFSSSWMNWVRQAPGKGLEWIGR |
| NO: 3164 | | H.32 | TYPGDGDTKYNGKFKGRATLSADKSKSTAYLQMNSLKTEDTAVYYCARRG |
| | | | TGGWYFDVWGQGTTVTVSS |
| SEQ ID | VH | E- | QVQLQESGPGLVKPSQTLSLTCAAYGYAFSSSWMNWVRQPPGKGLEWIGRI |
| NO: 3165 | | H.33 | YPGDGDTKYNGKFKGRATLSADKSKSQASLKLSSVTAADTAVYYCARRGT |
| | | | GGWYFDVWGQGTTVTVSS |
| SEQ ID | VH | E- | QVQLQESGSGLVKPSQTLSLTCAASGYAFSSSWMNWVRQPPGKGLEWIGRI |
| NO: 3166 | | H.34 | YPGDGDTKYNGKFKGRATLSADKSKSQASLKLSSVTAADTAVYYCARRGT |
| | | | GGWYFDVWGQGTTVTVSS |
| SEQ ID | VH | E- | EVQLVESGGGLVQPGGSLRLSCAASGYAFSSSWMNWVRQAPGKGLEWIGR |
| NO: 3167 | | H.35 | IYPGDGDTKYNGKFKGRATLSADKSKSSAYLQMNSLKTEDTAVYYCARRG |
| | | | TGGWYFDVWGQGTTVTVSS |
| SEQ ID | VH | E- | QVQLQESGPGLVKPSDTLSLTCTASGYAFSSSWMNWVRQPPGKGLEWIGRI |
| NO: 3168 | | H.36 | YPGDGDTKYNGKFKGRATLSADKSKSQASLKLSSVTAADTAVYYCARRGT |
| | | | GGWYFDVWGQGTTVTVSS |
| SEQ ID | VH | E- | QVQLQESGPGLVKPSQTLSLTCTASGYAFSSSWMNWVRQHPGKGLEWIGRI |
| NO: 3169 | | H.37 | YPGDGDTKYNGKFKGRATLSADKSKSQASLKLSSVTAADTAVYYCARRGT |
| | | | GGWYFDVWGQGTTVTVSS |
| SEQ ID | VH | E- | QVQLQESGPGLVKPSQTLSLTCTASGYAFSSSWMNWVRQHPGKGLEWIGRI |
| NO: 3170 | | H.38 | YPGDGDTKYNGKFKGLATLSADKSKSQASLKLSSVTAADTAVYYCARRGT |
| | | | GGWYFDVWGQGTTVTVSS |
| SEQ ID | VH | E- | QVQLVESGGGVVQPGRSLRLSCAASGYAFSSSWMNWVRQAPGKGLEWIGR |
| NO: 3171 | | H.39 | IYPGDGDTKYNGKFKGRATLSADKSKSTAYLQMSSLRAEDTAVYYCARRG |
| | | | TGGWYFDVWGQGTTVTVSS |
| SBQ ID | VH | E- | QVQLVESGGGLVKPGGSLRLSCAASGYAFSSSWMNWVRQAPGKGLEWIGR |
| NO: 3172 | | H.40 | IYPGDGDTKYNGKFKGRATLSADKAKSSAYLQMNSLRAEDTAVYYCARRG |
| | | | TGGWYFDVWGQGTTVTVSS |
| SEQ ID | VH | E- | QVQLVESGGGLVQPGGSLRLSCSASGYAFSSSWMNWVRQAPGKGLEWIGR |
| NO: 3173 | | H.41 | IYPGDGDTKYNGKFKGRATLSADKSKSTAYLQMNSLRAEDTAVYYCARRG |
| | | | TGGWYFDVWGQGTTVTVSS |
| SEQ ID | VH | E- | QVQLLESGGGLVKPGGSLRLSCAASGYAFSSSWMNWVRQAPGKGLEWIGR |
| NO: 3174 | | H.42 | IYPGDGDTKYNGKFKGRATLSADKAKSSAYLQMNSLRAEDTAVYYCARRG |
| | | | TGGWYFDVWGQGTTVTVSS |
| SEQ ID | VH | E- | EVQLVESGGGLVQPGGSLRLSCSASGYAFSSSWMNWVRQAPGKGLEWIGRI |
| NO: 3175 | | H.43 | YPGDGDTKYNGKFKGRATLSADKSKSTAYLQMSSLRAEDTAVYYCARRGT |
| | | | GGWYFDVWGQGTTVTVSS |
| SEQ ID | VH | E- | QVQLQESGPGLVKPSDTLSLTCAASGYAFSSSWMNWVRQPPGKGLEWIGRI |
| NO: 3176 | | H.44 | YPGDGDTKYNGKFKGRATLSADKSKSQASLKLSSVTAVDTAVYYCARRGT |
| | | | GGWYFDVWGQGTTVTVSS |
| SEQ ID | VH | E- | QVQLQESGPGLVKPSQTLSLTCAASGYAFSSSWMNWVRQPPGKGLEWIGRI |
| NO: 3177 | | H.45 | YPGDGDTKYNGKFKGRATLSADKSKSQASLKLSSVTAVDTAVYYCARRGT |
| | | | GGWYFDVWGQGTTVTVSS |
| SEQ ID | VH | E- | EVQLVESGGGLVQPGGSLRLSCSASGYAFSSSWMNWVRQAPGKGLEWIGRI |
| NO: 3178 | | H.46 | YPGDGDTKYNGKFKGRATLSADKSKSTAYVQMSSLRAEDTAVYYCARRGT |
| | | | GGWYFDVWGQGTTVTVSS |
| SEQ ID | VH | E- | QVQLVDSGGGVVQPGRSLRLSCAASGYAFSSSWMNWVRQAPGKGLEWIG |
| NO: 3179 | | H.47 | RIYPGDGDTKYNGKFKGRATLSADKSKSTAYLQMNSLRAEDTAVYYCARR |
| | | | GTGGWYFDVWGQGTTVTVSS |
| SEQ ID | VH | E- | QVQLVESGGGVVQPGRSLRLSCAASGYAFSSSWMNWVRQAPGKGLEWIGR |
| NO: 3180 | | H.48 | IYPGDGDTKYNGKFKGRATLSADKSKSTAYLQMNSLRAEGTAVYYCARRG |
| | | | TGGWYFDVWGQGTTVTVSS |
| SEQ ID | VH | E- | QVQLVESGGGVVQPGRSLRLSCAASGYAFSSSWMNWVRQAPGKGLEWIGR |
| NO: 3181 | | H.49 | IYPGDGDTKYNGKFKGRAILSADKSKSTAYLQMNSLRAEDTAVYYCARRG |
| | | | TGGWYFDVWGQGTTVTVSS |
| SEQ ID | VH | E- | EVQLVESGGGLVQPGGSLRLSCAASGYAFSSSWMNWVRQAPGKGLEWIGR |
| NO: 3182 | | H.50 | IYPGDGDTKYNGKFKGRATLSADKSKSTAYLQMNSLRAEDTAVYYCARRG |
| | | | TGGWYFDVWGQGTTVTVSS |

TABLE 12
Amino acid sequences for anti TCRβ V10 antibodies.
Amino acid and nucleotide sequences for murine and humanized
antibody molecules which bind to TCRBV 10 (e.g., TCRBV 10-1,
TCRBV 10-2 or TCRBV 10-3). The amino acid the heavy and
light chain CDRs, and the amino acid and nucleotide
sequences of the heavy and light chain variable regions,
and the heavy and light chains are shown.
Murine antibody D, also referred to as S511 antibody

| | | |
|---|---|---|
| SEQ ID NO: | HC CDR1 (Kabat) | SYGMS |
| 1288 | | |
| SEQ ID NO: | HC CDR2 (Kabat) | LISSGGSYTYYTDSVKG |
| 1289 | | |
| SEQ ID NO: | HC CDR3 (Kabat) | HGGNFFDY |
| 1290 | | |
| SEQ ID NO: | HC CDR1 (Chothia) | GFTFRSY |
| 1291 | | |
| SEQ ID NO: | HC CDR2 (Chothia) | SSGGSY |
| 1292 | | |
| SEQ ID NO: | HC CDR3 (Chothia) | HGGNFFDY |
| 1290 | | |
| SEQ ID NO: | HC CDR1 (Combined) | GFTFRSYGMS |
| 1293 | | |
| SEQ ID NO: | HC CDR2 (Combined) | LISSGGSYTYYTDSVKG |
| 1289 | | |
| SEQ ID NO: | HC CDR3 (Combined) | HGGNFFDY |
| 1290 | | |
| SEQ ID NO: | LC CDR1 (Kabat) | SVSSSVSYMH |
| 1294 | | |
| SEQ ID NO: | LC CDR2 (Kabat) | DTSKLAS |
| 1295 | | |
| SEQ ID NO: | LC CDR3 (Kabat) | QQWSSNPQYT |
| 1296 | | |
| SEQ ID NO: | LC CDR1 (Chothia) | SSSVSY |
| 1297 | | |
| SEQ ID NO: | LC CDR2 (Chothia) | DTSKLAS |
| 1295 | | |
| SEQ ID NO: | LC CDR3 (Chothia) | QQWSSNPQYT |
| 1296 | | |
| SEQ ID NO: | LC CDR1 (Combined) | SVSSSVSYMH |
| 1294 | | |
| SEQ ID NO: | LC CDR2 (Combined) | DTSKLAS |
| 1295 | | |
| SEQ ID NO: | LC CDR3 (Combined) | QQWSSNPQYT |
| 1296 | | |
| SEQ ID NO: | VH | EVQLVESGGDLVKPGGSLKLSCAVSGFTFRSYGMSWVRQT |
| 3183 | | PDKRLEWVALISSGGSYTYYTDSVKGRFTISRDNAKNTLYL |
| | | QMSSLKSEDTAIYYCSRHGGNFFDYWGQGTTLTVSS |
| SEQ ID NO: | VL | QIVLTQSPSIMSASPGEKVTMTCSVSSSVSYMHWYQQKSGT |
| 3184 | | SPKRWIYDTSKLASGVPARFSGSGSGTSYSLTISSMEAEDAA |
| | | TYYCQQWSSNPQYTFGGGTKLEIK |

Humanized antibody D (D-H antibody)
Variable light chain (VL)

| | | | |
|---|---|---|---|
| SEQ ID | VL | D-H.1 | DIVLTQSPAFLSVTPGEKVTITCSVSSSVSYMHWYQQKPDQAPKLLIYDTSK |
| NO: 3185 | | | LASGVPSRFSGSGSGTDYTFTISSLEAEDAATYYCQQWSSNPQYTFGQGTK |
| | | | LEIK |
| SEQ ID | VL | D-H.2 | AIQLTQSPSSLSASVGDRVTITCSVSSSVSYMHWYQQKPGKAPKLLIYDTSK |
| NO: 3186 | | | LASGVPSRFSGSGSGTDYTLTISSLQPEDFATYYCQQWSSNPQYTFGQGTKL |
| | | | EIK |
| SEQ ID | VL | D-H.3 | DIQLTQSPSFLSASVGDRVTITCSVSSSVSYMHWYQQKPGKAPKLLIYDTSK |
| NO: 3187 | | | LASGVPSRFSGSGSGTEYTLTISSLQPEDFATYYCQQWSSNPQYTFGQGTKL |
| | | | EIK |
| SEQ ID | VL | D-H.4 | DIQLTQSPSSLSASVGDRVTITCSVSSSVSYMHWYQQKPGKAPKLLIYDTSK |
| NO: 3188 | | | LASGVPSRFSGSGSGTDYTLTISSLQPEDFATYYCQQWSSNPQYTFGQGTKL |
| | | | EIK |
| SEQ ID | VL | D-H.5 | DIQLTQSPSSVSASVGDRVTITCSVSSSVSYMHWYQQKPGKAPKLLIYDTSK |
| NO: 3189 | | | LASGVPSRFSGSGSGTDYTLTISSLQPEDFATYYCQQWSSNPQYTFGQGTKL |
| | | | EIK |
| SEQ ID | VL | D-H.6 | DIQLTQSPSSLSASVGDRVTITCSVSSSVSYMHWYQQKPGKVPKLLIYDTSK |
| NO: 3190 | | | LASGVPSRFSGSGSGTDYTLTISSLQPEDVATYYCQQWSSNPQYTFGQGTK |
| | | | LEIK |
| SEQ ID | VL | D-H.7 | DIQLTQSPSSLSASVGDRVTITCSVSSSVSYMHWYQQKPGQAPKLLIYDTSK |
| NO: 3191 | | | LASGVPSRFSGSGSGTDYTLTISSLQPEDVATYYCQQWSSNPQYTFGQGTK |
| | | | LEIK |
| SEQ ID | VL | D-H.8 | EIVLTQSPDFQSVTPKEKVTITCSVSSSVSYMHWYQQKPDQSPKLLIYDTSK |
| NO: 3192 | | | LASGVPSRFSGSGSGTDYTLTINSLEAEDAATYYCQQWSSNPQYTFGQGTK |
| | | | LEIK |
| SEQ ID | VL | D-H.9 | AIRLTQSPFSLSASVQDRVTITCSVSSSVSYMHWYQQKPAKAPKLFIYDTSK |
| NO: 3193 | | | LASGVPSRFSGSGSGTDYTLTISSLQPEDFATYYCQQWSSNPQYTFGQGTKL |
| | | | EIK |
| SEQ ID | VL | D-H.10 | DIQLTQSPSSLSASVGDRVTITCSVSSSVSYMHWYQQKPGKAPKLLIYDTSK |
| NO: 3194 | | | LASGVPSRFSGSGSGTDYTFTISSLQPEDIATYYCQQWSSNPQYTFGQGTKL |
| | | | EIK |
| SEQ ID | VL | D-H.11 | EIVLTQSPATLSLSPGERATLSCSVSSSVSYMHWYQQKPGQAPKLLIYDTSK |
| NO: 3195 | | | LASGIPARFSGSGSGTDYTLTISSLEPEDFAVYYCQQWSSNPQYTFGQGTKL |
| | | | EIK |
| SEQ ID | VL | D-H.12 | DIQLTQSPSTLSASVGDRVTITCSVSSSVSYMHWYQQKPGKAPKLLIYDTSK |
| NO: 3196 | | | LASGVPSRFSGSGSGTEYTLTISSLQPDDFATYYCQQWSSNPQYTFGQQTKL |
| | | | EIK |
| SEQ ID | VL | D-H.13 | DIQLTQSPSSLSASVGDRVTITCSVSSSVSYMHWYQQKPGKTPKLLIYDTSK |
| NO: 3197 | | | LASGIPSRFSGSGSGTDYTLTIRSLQPEDFATYYCQQWSSNPQYTFGQGTKL |
| | | | EIK |
| SEQ ID | VL | D-H.14 | EIVLTQSPPTLSLSPGERVTLSCSVSSSVSYMHWYQQKPGQAPKLLIYDTSK |
| NO: 3198 | | | LASGIPARFSGSGSGTDYTLTISSLQPEDFAVYYCQQWSSNPQYTFGQGTKL |
| | | | EIK |
| SEQ ID | VL | D-H.15 | DIQLTQSPSSLSASVGDRVTITCSVSSSVSYMHWYQQKPGKAPKRLIYDTSK |
| NO: 3199 | | | LASGVPSRFSGSGSGTEYTLTISSLQPEDFATYYCQQWSSNPQYTFGQGTKL |
| | | | EIK |
| SEQ ID | VL | D-H.16 | EIVLTQSPATLSLSPGERATLSCSVSSSVSYMHWYQQKPGQAPKLLIYDTSK |
| NO: 3200 | | | LASGIPARFSGSGPGTDYTLTISSLEPEDFAVYYCQQWSSNPQYTFGQGTKL |
| | | | EIK |
| SEQ ID | VL | D-H.17 | EIVLTQSPATLSLSPGERATLSCSVSSSVSYMHWYQQKPGQAPKLLIYDTSK |
| NO: 3201 | | | LASGIPARFSGSGSGTDYTLTISRLEPEDFAVYYCQQWSSNPQYTFGQGTKL |
| | | | EIK |
| SEQ ID | VL | D-H.18 | EIVLTQSPATLSLSPGERATLSCSVSSSVSYMHWYQQKPGQAPKLLIYDTSK |
| NO: 3202 | | | LASGIPARFSGSGSGTDYTLTISSLQPEDFAVYYCQQWSSNPQYTFGQGTKL |
| | | | EIK |
| SEQ ID | VL | D-H.19 | EIVLTQSPATLSVSPGERATLSCSVSSSVSYMHWYQQKPGQAPKLLIYDTSK |
| NO: 3203 | | | LASGIPARFSGSGSGTEYTLTISSLQSEDFAVYYCQQWSSNPQYTFGQGTKL |
| | | | EIK |
| SEQ ID | VL | D-H.20 | EIVLTQSPATLSVSPGERATLSCSVSSSVSYMHWYQQKPGQAPKLLIYDTSK |
| NO: 3204 | | | LASGIPARFSGSGSGTEYTLTISILQSEDFAVYYCQQWSSNPQYTFGQGTKL |
| | | | EIK |
| SEQ ID | VL | D-H.21 | EIVLTQSPPTLSLSPGERVTLSCSVSSSVSYMHWYQQKPGQAPKLLIYDTSK |
| NO: 3205 | | | LASSIPARFSGSGSGTDYTLTISSLQPEDFAVYYCQQWSSNPQYTFGQGTKL |
| | | | EIK |
| SEQ ID | VL | D-H.22 | DIQLTQSPSSLSASVGDRVTITCSVSSSVSYMHWYQQKPGKAPKSLIYDTSK |
| NO: 3206 | | | LASGVPSRFSGSGSGTDYTLTISSLQPEDFATYYCQQWSSNPQYTFGQGTKL |
| | | | EIK |
| SEQ ID | VL | D-H.23 | DIQLTQSPSSLSASVGDRVTITCSVSSSVSYMHWYQQKPGKAPKRLIYDTSK |
| NO: 3207 | | | LASGVPSRFSGSGSGTEYTLTISNLQPEDFATYYCQQWSSNPQYTFGQGTKL |
| | | | EIK |
| SEQ ID | VL | D-H.24 | DIQLTQSPSAMSASVGDRVTITCSVSSSVSYMHWYQQKPGKVPKRLIYDTS |
| NO: 3208 | | | KLASGVPSRFSGSGSGTEYTLTISSLQPEDFATYYCQQWSSNPQYTFGQGTK |
| | | | LEIK |
| SEQ ID | VL | D-H.25 | EIVLTQSPATLSLSPGERATLSCSVSSSVSYMHWYQQKPGQAPKLLIYDTSK |
| NO: 3209 | | | LASGIPDRFSGSGSGTDYTLTISRLEPEDFAVYYCQQWSSNPQYTFGQGTKL |
| | | | EIK |
| SEQ ID | VL | D-H.26 | EIVLTQSPATLSLSPGERATLSCSVSSSVSYMHWYQQKPGLAPKLLIYDTSK |
| NO: 3210 | | | LASGIPDRFSQSGSGTDYTLTISRLEPEDFAVYYCQQWSSNPQYTFGQGTKL |
| | | | EIK |
| SEQ ID | VL | D-H.27 | EIVLTQSPGTLSLSPGERATLSCSVSSSVSYMHWYQQKPGQAPKLLIYDTSK |
| NO: 3211 | | | LASGIPDRFSGSGSGTDYTLTISRLEPEDFAVYYCQQWSSNPQYTFGQGTKL |
| | | | EIK |
| SEQ ID | VL | D-H.28 | DIQLTQSPSSLSASVGDRVTITCSVSSSVSYMHWYQQKPGKAPKSLIYDTSK |
| NO: 3212 | | | LASGVPSKFSGSGSGTDYTLTISSLQPEDFATYYCQQWSSNPQYTFGQGTKL |
| | | | EIK |
| SEQ ID | VL | D-H.29 | DIQLTQSPSSLSASVGDRVTITCSVSSSVSYMHWYQQKPEKAPKSLIYDTSK |
| NO: 3213 | | | LASGVPSRFSGSGSGTDYTLTISSLQPEDFATYYCQQWSSNPQYTFGQGTKL |
| | | | EIK |
| SEQ ID | VL | D-H.30 | DIVLTQSPDSLAVSLGERATINCSVSSSVSYMHWYQQKPGQPPKLLIYDTSK |
| NO: 3214 | | | LASGVPDRFSGSGSGTDYTLTISSLQAEDVAVYYCQQWSSNPQYTFGQGTK |
| | | | LEIK |
| SEQ ID | VL | D-H.31 | EIVLTQTPLSLSITPGEQASMSCSVSSSVSYMHWYLQKARPVPKLLIYDTSK |
| NO: 3215 | | | LASGVPDRFSGSGSGTDYTLKISRVEAEDFGVYYCQQWSSNPQYTFGQGTK |
| | | | LEIK |
| SEQ ID | VL | D-H.32 | EIVLTQTPLSLSITPGEQASISCSVSSSVSYMHWYLQKARPVPKLLIYDTSKL |
| NO: 3216 | | | ASGVPDRFSGSGSGTDYTLKISRVEAEDFGVYYCQQWSSNPQYTFGQGTKL |
| | | | EIK |
| SEQ ID | VL | D-H.33 | DIVLTQSPLSLPVTPGEPASISCSVSSSVSYMHWYLQKPGQSPKLLIYDTSKL |
| NO: 3217 | | | ASGVPDRFSGSGSGTDYTLKISRVEAEDVGVYYCQQWSSNPQYTFGQGTK |
| | | | LEIK |
| SEQ ID | VL | D-H.34 | DIVLTQSPLSLPVTLGQPASISCSVSSSVSYMHWYQQRPGQSPKRLIYDTSK |
| NO: 3218 | | | LASGVPDRFSGSGSGTDYTLKISRVEAEDVGVYYCQQWSSNPQYTFGQGT |
| | | | KLEIK |
| SEQ ID | VL | D-H.35 | DIVLTQTPLSLPVTPGEPASISCSVSSSVSYMHWYLQKPGQSPKLLIYDTSKL |
| NO: 3219 | | | ASGVPDRFSGSGSGTDYTLKISRVEAEDVGVYYCQQWSSNPQYTFGQGTK |
| | | | LEIK |
| SEQ ID | VL | D-H.36 | DIVLTQTPLSLSVTPGQPASISCSVSSSVSYMHWYLQKPGQSPKLLIYDTSKL |
| NO: 3220 | | | ASGVPDRFSGSGSGTDYTLKISRVEAEDVGVYYCQQWSSNPQYTFGQGTK |
| | | | LEIK |
| SEQ ID | VL | D-H.37 | DIVLTQTPLSLSVTPGQPASISCSVSSSVSYMHWYLQKPGQPPKLLIYDTSKL |
| NO: 3221 | | | ASGVPDRFSGSGSGTDYTLKISRVEAEDVGVYYCQQWSSNPQYTFGQGTK |
| | | | LEIK |
| SEQ ID | VL | D-H.38 | DIQLIQSPSFLSASVGDRVSIICSVSSSVSYMHWYLQKPGKSPKLFIYDTSKL |
| NO: 3222 | | | ASGVSSRFSGRGSGTDYTLTIISLKPEDFAAYYCQQWSSNPQYTFGQGTKLE |
| | | | IK |
| SEQ ID | VL | D-H.39 | DIVLTQTPLSSPVTLGQPASISCSVSSSVSYMHWYQQRPGQPPKLLIYDTSKL |
| NO: 3223 | | | ASGVPDRFSGSGAGTDYTLKISRVEAEDVGVYYCQQWSSNPQYTFGQGTK |
| | | | LEIK |
| SEQ ID | VL | D-H.40 | EITLTQSPAFMSATPGDKVNISCSVSSSVSYMHWYQQKPGEAPKFHYDTSK |
| NO: 3224 | | | LASGIPPRFSGSGYGTDYTLTINNIESEDAAYYYCQQWSSNPQYTFGQGTKL |
| | | | EIK |

Variable HEAVY chain (VH)

| | | | |
|---|---|---|---|
| SEQ ID | VH | D-H.1 | EVQLVESGGGLVKPGGSLRLSCAVSGFTFRSYGMSWVRQAPGKGLEWVA |
| NO: 3225 | | | LISSGGSYTYYTDSVKGRFTISRDNSKNTLYLQMNSLKTEDTAVYYCSRHG |
| | | | GNFFDYWGQGTTVTVSS |
| SEQ ID | VH | D-H.2 | EVQLVESGGALVKPGGSLRLSCAVSGFTFRSYGMSWVRQAPGKGLEWVA |
| NO: 3226 | | | LISSGGSYTYYTDSVKGRFTISRDNSKNTLYLQMNSLKTEDTAVYYCSRHG |
| | | | GNFFDYWGQQTTVTVSS |
| SEQ ID | VH | D-H.3 | EVQLVESGGGLVQPGGSLRLSCAVSGFTFRSYGMSWVRQAPGKGLEWVA |
| NO: 3227 | | | LISSGGSYTYYTDSVKQRFTISRDNAKNTLYLQMNSLRAEDTAVYYCSRHG |
| | | | GNFFDYWGQGTTVTVSS |
| SEQ ID | VH | D-H.4 | EVQLVESGGGLVQPGGSLRLSCAVSGFTFRSYGMSWVRQAPGKGLEWVA |
| NO: 3228 | | | LISSGGSYTYYTDSVKGRFTISRDNAKNSLYLQMNSLRAEDTAVYYCSRHG |
| | | | GNFFDYWGQGTTVTVSS |
| SEQ ID | VH | D-H.5 | EVQLVESGGGLVQPGGSLRLSCAVSGFTFRSYGMSWVRQAPGKGLEWVA |
| NO: 3229 | | | LISSGGSYTYYTDSVKGRFTISRDNSKNSLYLQMNSLKTEDTAVYYCSRHG |
| | | | GNFFDYWGQGTTVTVSS |
| SEQ ID | VH | D-H.6 | EVQLVESGGGLVQPGGSLRLSCAVSGFTFRSYGMSWVRQAPGKGLEWVA |
| NO: 3230 | | | LISSGGSYTYYTDSVKGRFTISRDNAKNSLYLQMNSLRAEDMAVYYCSRH |
| | | | GGNFFDYWGQGTTVTVSS |
| SEQ ID | VH | D-H.7 | EVQLVESGGGLVQPGGSLRLSCAVSGFTFRSYGMSWVRQAPGKGLEWVA |
| NO: 3231 | | | LISSGGSYTYYTDSVKGQFTISRDNAKNTLYLQMNSLRAEDMAVYYCSRH |
| | | | GGNFFDYWGQGTTVTVSS |
| SEQ ID | VH | D-H.8 | EVQLVESGGGLVKPGRSLRLSCTVSGFTFRSYGMSWVRQAPGKGLEWVAL |
| NO: 3232 | | | ISSGGSYTYYTDSVKGRFTISRDNSKNILYLQMNSLKTEDTAVYYCSRHGG |
| | | | NFFDYWGQGTTVTVSS |
| SEQ ID | VH | D-H.9 | EVQLVESGGGLVKPGGSLRLSCAVSGFTFRSYGMSWVRQAPGKGLEWVA |
| NO: 3233 | | | LISSGGSYTYYTDSVKGRFTISRDNAKNSLYLQMNSLRAEDTAVYYCSRHG |
| | | | GNFFDYWGQGTTVTVSS |
| SEQ ID | VH | D-H.10 | EVQLVESGGGLVQPGGSLKLSCAVSGFTFRSYGMSWVRQASGKGLEWVA |
| NO: 3234 | | | LISSGGSYTYYTDSVKGRFTISRDNSKNTLYLQMNSLKTEDTAVYYCSRHG |
| | | | GNFFDYWGQGTTVTVSS |
| SEQ ID | VH | D-H.11 | QVQLVESGGGVVQPGGSLRLSCAVSGFTFRSYGMSWVRQAPGKGLEWVA |
| NO: 3235 | | | LISSGGSYTYYTDSVKGRFTISRDNSKNTLYLQMNSLRAEDTAVYYCSRHG |
| | | | GNFFDYWGQGTTVTVSS |
| SEQ ID | VH | D-H.12 | QVQLVESGGGVVQPGRSLRLSCAVSGFTFRSYGMSWVRQAPGKGLEWVA |
| NO: 3236 | | | LISSGGSYTYYTDSVKGRFTISRDNSKNTLYLQMSSLRAEDTAVYYCSRHG |
| | | | GNFFDYWGQGTTVTVSS |
| SEQ ID | VH | D-H.13 | EVQLVESGGGLVQPGGSLRLSCPVSGFTFRSYGMSWVRQAPGKGLEWVAL |
| NO: 3237 | | | ISSGGSYTYYTDSVKGRFTISRDNANNSLYLQMNSLRAEDTAVYYCSRHGG |
| | | | NFFDYWGQGTTVTVSS |
| SEQ ID | VH | D-H.14 | EVQLVESGGGLVQPGRSLRLSCTVSGFTFRSYGMSWVRQAPGKGLEWVAL |
| NO: 3238 | | | ISSGGSYTYYTDSVKGRFTISRDNSKNILYLQMNSLKTEDTAVYYCSRHGG |
| | | | NFFDYWGQGTTVTVSS |
| SEQ ID | VH | D-H.15 | EVQLVESGGGLVQPGPSLRLSCTVSGFTFRSYGMSWVRQAPGKGLEWVAL |
| NO: 3239 | | | ISSGGSYTYYTDSVKGRFTISRDNSKNILYLQMNSLKTEDTAVYYCSRHGG |
| | | | NFFDYWGQGTTVTVSS |
| SEQ ID | VH | D-H.16 | EVQLVESGGGLVQPGGSLRLSCAVSGFTFRSYGMSWVRQAPGKGLEWVA |
| NO: 3240 | | | LISSGGSYTYYTDSVKGRFTISRDNSKNTLYLQMNSLRAEDTAVYYCSRHG |
| | | | GNFFDYWGQGTTVTVSS |
| SEQ ID | VH | D-H.17 | EVQLVESGGGLVQPGGSLRLSCAVSGFTFRSYGMSWVRQAPGKGLEWVA |
| NO: 3241 | | | LISSGGSYTYYTDSVKGRFTISRDNAKNSLYLQMNSLRDEDTAVYYCSRHG |
| | | | GNFFDYWGQGTTVTVSS |
| SEQ ID | VH | D-H.18 | QVQLVESGGGLVKPGGSLRLSCAVSGFTFRSYGMSWVRQAPGKGLEWVA |
| NO: 3242 | | | LISSGGSYTYYTDSVKGRFTISRDNAKNSLYLQMNSLRAEDTAVYYCSRHG |
| | | | GNFFDYWGQGTTVTVSS |
| SEQ ID | VH | D-H.19 | QVQLVESGGGVVQPGRSLRLSCAVSGFTFRSYGMSWVRQAPGKGLEWVA |
| NO: 3243 | | | LISSGGSYTYYTDSVKGRFTISRDNSKNTLYLQMNSLRAEDTAVYYCSRHG |
| | | | GNFFDYWGQGTTVTVSS |
| SEQ ID | VH | D-H.20 | EVQLLESGGGLVQPGGSLRLSCAVSGFTFRSYGMSWVRQAPGKGLEWVAL |
| NO: 3244 | | | ISSGGSYTYYTDSVKGRFTISRDNSKNTLYLQMNSLRAEDTAVYYCSRHGG |
| | | | NFFDYWGQGTTVTVSS |
| SEQ ID | VH | D-H.21 | EVQLVESGGGLVQPGGSLRLSCAVSGFTFRSYGMSWVRQAPGKGLEWVA |
| NO: 3245 | | | LISSGGSYTYYTDSVKGRFTISRHNSKNTLYLQMNSLRAEDTAVYYCSRHG |
| | | | GNFFDYWGQGTTVTVSS |
| SEQ ID | VH | D-H.22 | EVQLVESGGGLIQPGGSLRLSCAVSGFTFRSYGMSWVRQPPGKGLEWVALI |
| NO: 3246 | | | SSGGSYTYYTDSVKGRFTISRDNSKNTLYLQMNSLRAEDTAVYYCSRHGG |
| | | | NFFDYWGQGTTVTVSS |
| SEQ ID | VH | D-H.23 | EVQLVESGGGLIQPGGSLRLSCAVSGFTFRSYGMSWVRQAPGKGLEWVAL |
| NO: 3247 | | | ISSGGSYTYYTDSVKGRFTISRDNSKNTLYLQMNSLRAEDTAVYYCSRHGG |
| | | | NFFDYWGQGTTVTVSS |
| SEQ ID | VH | D-H.24 | EVQLVESGGGLVQPGRSLRLSCAVSGFTFRSYGMSWVRQAPGKGLEWVA |
| NO: 3248 | | | LISSGGSYTYYTDSVKGRFTISRDNAKNSLYLQMNSLRAEDTALYYCSRHG |
| | | | GNFFDYWGQGTTVTVSS |
| SEQ ID | VH | D-H.25 | QVQLVESGGGVVQPGRSLRLSCAVSGFTFRSYGMSWVRQAPGKGLEWVA |
| NO: 3249 | | | LISSGGSYTYYTDSVKGRFTISRDNSKNRLYLQMNSLRAEDTAVYYCSRHG |
| | | | GNFFDYWGQGTTVTVSS |
| SEQ ID | VH | D-H.26 | QVQLVESGGGVVQPGRSLRLSCAVSGFTFRSYGMSWVRQAPGKGLEWVA |
| NO: 3250 | | | LISSGGSYTYYTDSVKGRFTISRDNSKNTLYLQMNSLRAEGTAVYYCSRHG |
| | | | GNFFDYWGQGTTVTVSS |
| SEQ ID | VH | D-H.27 | QVQLVESGGGVVQPGRSLRLSCAVSGFTFRSYGMSWVRQAPGKGLEWVA |
| NO: 3251 | | | LISSGGSYTYYTDSVKGRFAISRDNSKNTLYLQMNSLRAEDTAVYYCSRHG |
| | | | GNFFDYWGQGTTVTVSS |
| SEQ ID | VH | D-H.28 | QVQLVDSGGGVVQPGRSLRLSCAVSGFTFRSYGMSWVRQAPGKGLEWVA |
| NO: 3252 | | | LISSGGSYTYYTDSVKGRFTISRDNSKNTLYLQMNSLRAEDTAVYYCSRHG |
| | | | GNFFDYWGQGTTVTVSS |
| SEQ ID | VH | D-H.29 | EVQLVESGGGVVRPGGSLRLSCAVSGFTFRSYGMSWVRQAPGKGLEWVA |
| NO: 3253 | | | LISSGGSYTYYTDSVKGRFTISRDNAKNSLYLQMNSLRAEDTALYHCSRHG |
| | | | GNFFDYWGQGTTVTVSS |
| SEQ ID | VH | D-H.30 | EVQLVESGGVVVQPGGSLRLSCAVSGFTFRSYGMSWVRQAPGKGLEWVA |
| NO: 3254 | | | LISSGGSYTYYTDSVKGRFTISRDNSKNSLYLQMNSLRAEDTALYYCSRHG |
| | | | GNFFDYWGQGTTVTVSS |
| SEQ ID | VH | D-H.31 | EVQLVESGGGVVQPGGSLRLSCAVSGFTFRSYGMSWVRQAPGKGLEWVA |
| NO: 3255 | | | LISSGGSYTYYTDSVKGRFTISRDNSKNSLYLQMNSLRTEDTALYYCSRHG |
| | | | GNFFDYWGQGTTVTVSS |
| SEQ ID | VH | D-H.32 | EVQLVESGGVVVQPGGSLRLSCAVSGFTFRSYGMSWVRQAPGKGLEWVA |
| NO: 3256 | | | LISSGGSYTYYTDSVKGRFTISRDNSKNSLYLQMNSLRTEDTALYYCSRHG |
| | | | GNFFDYWGQGTTVTVSS |
| SEQ ID | VH | D-H.33 | EVQLVETGGGLIQPGGSLRLSCAVSGFTFRSYGMSWVRQAPGKGLEWVAL |
| NO: 3257 | | | ISSGGSYTYYTDSVKGRFTISRDNSKNTLYLQMNSLRAEDTAVYYCSRHGG |
| | | | NFFDYWGQGTTVTVSS |
| SEQ ID | VH | D-H.34 | EVQLVESGGGLVQPGGSLRLSCAVSGFTFRSYGMSWVRQATGKGLEWVA |
| NO: 3258 | | | LISSGGSYTYYTDSVKGRFTISRENAKNSLYLQMNSLRAGDTAVYYCSRHG |
| | | | GNFFDYWGQGTTVTVSS |
| SEQ ID | VH | D-H.35 | EVQLVESRGVLVQPGGSLRLSCAVSGFTFRSYGMSWVRQAPGKGLEWVA |
| NO: 3259 | | | LISSGGSYTYYTDSVKGRFTISRDNSKNTLHLQMNSLRAEDTAVYYCSRHG |
| | | | GNFFDYWGQGTTVTVSS |
| SEQ ID | VH | D-H.36 | EVQLVESGGGLVQPGRSLRLSCAVSGFTFRSYGMSWVRQAPGKGLEWVA |
| NO: 3260 | | | LISSGGSYTYYTDSVKGRFTISRDNAKNSLYLQMNSLRAEDMALYYCSRHG |
| | | | GNFFDYWGQGTTVTVSS |
| SEQ ID | VH | D-H.37 | QVQLVESGQGLVQPGGSLRLSCSVSQFTFRSYGMSWVRQAPGKGLEWVA |
| NO: 3261 | | | LISSGGSYTYYTDSVKGRFTISRDNSKNTLYLQMNSLRAEDTAVYYCSRHG |
| | | | GNFFDYWGQGTTVTVSS |
| SEQ ID | VH | D-H.38 | EVQLVESGGGLVQPGGSLRLSCSVSGFTFRSYGMSWVRQAPGKGLEWVAL |
| NO: 3262 | | | ISSGGSYTYYTDSVKGRFTISRDNSKNTLYLQMSSLRAEDTAVYYCSRHGG |
| | | | NFFDYWGQGTTVTVSS |
| SEQ ID | VH | D-H.39 | QVQLVESGGGVVQPGRSLRLSCAVSGFTFRSYGMSWVRQAPGKGLEWVA |
| NO: 3263 | | | LISSGGSYTYYTDSVKGRFTISRDNSTNTLFLQMNSLRAEDTAVYYCSRHG |
| | | | GNFFDYWGQGTTVTVSS |
| SEQ ID | VH | D-H.40 | QVQLLESGGGLVKPGGSLRLSCAVSGFTFRSYGMSWVRQAPGKGLEWVA |
| NO: 3264 | | | LISSGGSYTYYTDSVKGRFTISRDNAKNSLYLQMNSLRAEDTAVYYCSRHG |
| | | | GNFFDYWGQGTTVTVSS |
| SEQ ID | VH | D-H.41 | EVQLVESGEGLVQPGGSLRLSCAVSGFTFRSYGMSWVRQAPGKGLEWVAL |
| NO: 3265 | | | ISSGGSYTYYTDSVKGRFTISRDNSKNTLYLQMGSLRAEDMAVYYCSRHG |
| | | | GNFFDYWGQGTTVTVSS |
| SEQ ID | VH | D-H.42 | EVQLVESGGGLVQPGGSLRLSCAVSGFTFRSYGMSWVRQAPGKGLEWVA |
| NO: 3266 | | | LISSGGSYTYYTDSVKQRFTISRDNSKNTLYLQMGSLRAEDMAVYYCSRHG |
| | | | GNFFDYWGQGTTVTVSS |
| SEQ ID | VH | D-H.43 | EVQLVESGGGLVQPGGSLRLSCSVSGFTFRSYGMSWVRQAPGKGLEWVAL |
| NO: 3267 | | | ISSGGSYTYYTDSVKGRFTISRDNSKNTLYVQMSSLRAEDTAVYYCSRHGG |
| | | | NFFDYWGQGTTVTVSS |
| SEQ ID | VH | D-H.44 | EVQLVESGGGLVQPGGSLRLSCAVSGFTFRSYGMSWVRQAPGKGLEWVA |
| NO: 3268 | | | LISSGGSYTYYTDSVKGRFIISRDNSRNSLYLQKNRRRAEDMAVYYCSRHG |
| | | | GNFFDYWGQGTTVTVSS |
| SEQ ID | VH | D-H.45 | EVQLVESGGGLVQPGGSLRLSCAVSGFTFRSYGMSWVHQAPGKGLEWVA |
| NO: 3269 | | | LISSGGSYTYYTDSVKGRFIISRDNSRNTLYLQTNSLRAEDTAVYYCSRHGG |
| | | | NFFDYWGQGTTVTVSS |
| SEQ ID | VH | D-H.46 | EVHLVESGGGLVQPGGALRLSCAVSGFTFRSYGMSWVRQATGKGLEWVA |
| NO: 3270 | | | LISSGGSYTYYTDSVKGRFTISRENAKNSLYLQMNSLRAGDTAVYYCSRHG |
| | | | GNFFDYWGQGTTVTVSS |
| SEQ ID | VH | D-H.47 | EVQLVESGGGLVQPRGSLRLSCAVSGFTFRSYGMSWVRQAPGKGLEWVA |
| NO: 3271 | | | LISSGGSYTYYTDSVKGRFTISRDNSKNTLYLQMNNLRAEGTAVYYCSRHG |
| | | | GNFFDYWGQGTTVTVSS |
| SEQ ID | VH | D-H.48 | EVQLVESGGGLVQPRGSLRLSCAVSGFTFRSYGMSWVRQAPGKGLEWVA |
| NO: 3272 | | | LISSGGSYTYYTDSVKGRFTISRDNSKNTLYLQMNNLRAEGTAAYYCSRHG |
| | | | GNFFDYWGQGTTVTVSS |
| SEQ ID | VH | D-H.49 | QVQLVQSGAEVKKPGASVKVSCKVSGFTFRSYGMSWVRQAPGKGLEWVA |
| NO: 3273 | | | LISSGGSYTYYTDSVKGRFTITRDNSTNTLYMELSSLRSEDTAVYYCSRHGG |
| | | | NFFDYWGQGTTVTVSS |
| SEQ ID | VH | D-H.50 | QVQLVQSGSELKKPGASVKVSCKVSGFTFRSYGMSWVRQAPGQGLEWVA |
| NO: 3274 | | | LISSGGSYTYYTDSVKGRFVISRDNSVNTLYLQISSLKAEDTAVYYCSRHGG |
| | | | NFFDYWGQGTTVTVSS |

TABLE 13
Amino acid sequences for additional anti-TCRβ V antibodies.
Amino acid and nucleotide sequences for murine and humanized
antibody molecules which bind to various TCRVB families are
disclosed. The amino acid the heavy and light chain CDRs,
and the amino acid and nucleotide sequences of the heavy and
light chain variable regions, and the heavy and light chains
are shown. Antibodies disclosed in the table include, MPB2D5,
CAS1.1.3, IMMU222, REA1062, and JOVI-3. MPB2D5 binds human
TCRβV 20-1 (TCRβV2 per old nomenclature). CAS1.1.3 binds human
TCRβV 27 (TCRβV14 per old nomenclature). IMMU222 binds human
TCRβV 6-5, TCRβV 6-6, or TCRβV 6-9 (TCRβV13.1 per old nomenclature).
REA1062 binds human TCRβV 5-1). JOVI-3 binds human TCRβV 28
(TCRβV3.1 per old nomenclature). IMMU546 binds human TCRβV 2.
MPB2DS (murine), also referred to here as BJM1104, BJ1188,
BJ1190 and REA654; or Antibody G Binds to human TCRVβ 20-1

| | | |
|---|---|---|
| SEQ ID NO: | HC CDR1 (Kabat) | SAYMH |
| 1102 | | |
| SEQ ID NO: | HC CDR2 (Kabat) | RIDPATGKTKYAPKFQA |
| 1103 | | |
| SEQ ID NO: | HC CDR3 (Kabat) | SLNWDYGLDY |
| 1104 | | |
| SEQ ID NO: | HC CDR1 (Chothia) | GFNIKSA |
| 1105 | | |
| SEQ ID NO: | HC CDR2 (Chothia) | DPATGK |
| 1106 | | |
| SEQ ID NO: | HC CDR3 (Chothia) | SLNWDYGLDY |
| 1104 | | |
| SEQ ID NO: | HC CDR1 (Combined) | GFNIKSAYMH |
| 3640 | | |
| SEQ ID NO: | HC CDR2 (Combined) | RIDPATGKTKYAPKFQA |
| 1103 | | |
| SEQ ID NO: | HC CDR3 (Combined) | SLNWDYGLDY |
| 1104 | | |
| SEQ ID NO: | LC CDR1 (Kabat) | RASKSVSILGTHLIH |
| 1107 | | |
| SEQ ID NO: | LC CDR2 (Kabat) | AASNLES |
| 1108 | | |
| SEQ ID NO: | LC CDR3 (Kabat) | QQSIEDPWT |
| 1109 | | |
| SEQ ID NO: | LC CDR1 (Chothia) | SKSVSILGTHL |
| 1110 | | |
| SEQ ID NO: | LC CDR2 (Chothia) | AASNLES |
| 1108 | | |
| SEQ ID NO: | LC CDR3 (Chothia) | QQSIEDPWT |
| 1109 | | |
| SEQ ID NO: | LC CDR1 (Combined) | RASKSVSILGTHLIH |
| 1107 | | |
| SEQ ID NO: | LC CDR2 (Combined) | AASNLES |
| 1108 | | |
| SEQ ID NO: | LC CDR3 (Combined) | QQSIEDPWT |
| 1109 | | |
| SEQ ID NO: | VL | DIVLTQSPASLAVSLGQRATISCRASKSVSILGTHLIHWYQQKP |
| 1111 | | GQPPKLLIYAASNLESGVPARFSGSGSETVFTLNIHPVEEEDAA |
| | | TYFCQQSIEDPWTFGGGTKLGIK |
| SEQ ID NO: | VH | EVQLQQSVADLVRPGASLKLSCTASGFNIKSAYMHWVIQRPD |
| 1112 | | QGPECLGRIDPATGKTKYAPKFQAKATITADTSSNTAYLQLSSL |
| | | TSEDTAIYYCTRSLNWDYGLDYWGQGTSVTVSS |

VH for MPB2D5 (humanized) also referred to as Antibody G-H (humanized)
Binds to human TCRVβ 20-1

| | | |
|---|---|---|
| SEQ ID NO: | VH-1 | QVQLVQSGAEVKKPGASVKVSCKASGFNIKSAYMHWVRQAP |
| 1113 | | GQGLEWMGRIDPATGKTKYAPKFQARVTMTADTSTNTAYME |
| | | LSSLRSEDTAVYYCARSLNWDYGLDYWGQGTLVTVSS |
| SEQ ID NO: | VH-2 | QVQLVQSGAEVKKPGASVKVSCKASGFNIKSAYMHWVRQAP |
| 1114 | | GQEPGCMGRIDPATGKTKYAPKFQARVTMTADTSINTAYTELS |
| | | SLRSEDTATYYCARSLNWDYGLDYWGQGTLVTVSS |
| SEQ ID NO: | VH-3 | QVQLVQSGAEVKKPGSSVKVSCKASGFNIKSAYMHWVRQAP |
| 1115 | | GQGLEWMGRIDPATGKTKYAPKFQARVTITADTSTNTAYMEL |
| | | SSLRSEDTAVYYCARSLNWDYGLDYWGQGTLVTVSS |
| SEQ ID NO: | VH-4 | QVQLVQSGAEVKKPGASVKVSCKASGFNIKSAYMHWVRQAP |
| 1116 | | GQRLEWMGRIDPATGKTKYAPKFQARVTITADTSANTAYMEL |
| | | SSLRSEDTAVYYCARSLNWDYGLDYWGQGTLVTVSS |

VL for MPB2D5 (humanized) also referred to as Antibody G-H (humanized)
Binds to human TCRVβ 20-1

| | | |
|---|---|---|
| SEQ ID NO: | VL-1 | EIVLTQSPATLSLSPGERATLSCRASKSVSILGTHLIHWYQQKPG |
| 1117 | | QAPRLLIYAASNLESGIPARFSGSGSETDFTLTISSLEPEDFAVYF |
| | | CQQSIEDPFGGGTKVEIK |
| SEQ ID NO: | VL-2 | EIVLTQSPATLSLSPGERATLSCRASKSVSILGTHLIHWYQQKPG |
| 1118 | | LAPRLLIYAASNLESGIPDRFSGSGSETDFTLTISRLEPEDFAVYF |
| | | CQQSIEDPFGGGTKVEIK |
| SEQ ID NO: | VL-3 | EIVLTQSPGTLSLSPGERATLSCRASKSVSILGTHLIHWYQQKPG |
| 1119 | | QAPRLLIYAASNLESGIPDRFSGSGSETDFTLTISRLEPEDFAVYF |
| | | CQQSIEDPFGGGTKVEIK |

CAS1.1.3 (murine) also referred to herein as BJ1460; or Antibody H
Binds to human TCRVβ 27

| | | |
|---|---|---|
| SEQ ID NO: | HC CDR1 (Kabat) | DTYMY |
| 1120 | | |
| SEQ ID NO: | HC CDR2 (Kabat) | RIDPANGNTKYDPKFQD |
| 1121 | | |
| SEQ ID NO: | HC CDR3 (Kabat) | GSYYYAMDY |
| 1122 | | |
| SEQ ID NO: | HC CDR1 (Chothia) | GFKTEDT |
| 1123 | | |
| SEQ ID NO: | HC CDR2 (Chothia) | DPANGN |
| 1124 | | |
| SEQ ID NO: | HC CDR3 (Chothia) | GSYYYAMDY |
| 1122 | | |
| SEQ ID NO: | HC CDR1 (Combined) | GFKTEDTYMY |
| 1125 | | |
| SEQ ID NO: | HC CDR2 (Combined) | RIDPANGNTKYDPKFQD |
| 1121 | | |
| SEQ ID NO: | HC CDR3 (Combined) | GSYYYAMDY |
| 1122 | | |
| SEQ ID NO: | LC CDR1 (Kabat) | RASESVDSYGNSFMH |
| 1126 | | |
| SEQ ID NO: | LC CDR2 (Kabat) | RASNLES |
| 1127 | | |
| SEQ ID NO: | LC CDR3 (Kabat) | QQSNEDPYT |
| 1128 | | |
| SEQ ID NO: | LC CDR1 (Chothia) | SESVDSYGNSF |
| 3641 | | |
| SEQ ID NO: | LC CDR2 (Chothia) | RASNLES |
| 1127 | | |
| SEQ ID NO: | LC CDR3 (Chothia) | QQSNEDPYT |
| 1128 | | |
| SEQ ID NO: | LC CDR1 (Combined) | RASESVDSYGNSFMH |
| 1126 | | |
| SEQ ID NO: | LC CDR2 (Combined) | RASNLES |
| 1127 | | |
| SEQ ID NO | LC CDR3 (Combined) | QQSNEDPYT |
| 1128 | | |
| SEQ ID NO: | VL | DIVLTQSPASLAVSLGQRATISCRASESVDSYGNSFMHWYQQK |
| 1129 | | PGQPPKLLIYRASNLESGIPARFSGSGSRTDFTLTINPVEADDVA |
| | | TYYCQQSNEDPYTFGGGTKLEIK |
| SEQ ID NO: | VH | EVQLQQSGAELVKPGASVKLSCTASGFKTEDTYMYWVKQRPE |
| 1130 | | QGLEWIGRIDPANGNTKYDPKFQDKATITADSSSNTAYLQLSS |
| | | LPSEDTAVYYCARGSYYYAMDYWGQGTSVTVSS |

VH for CAS1.1.3 (humanized) also referred to as Antibody H-H (humanized)
Binds to human TCRVβ 27

| | | |
|---|---|---|
| SEQ ID NO: | VH-1 | QVQLVQSGAEVKKPGSSVKVSCKASGFKTEDTYMYWVRQAP |
| 1131 | | GQGLEWIGRIDPANGNTKYDPKFQDRATITADSSTNTAYMELS |
| | | SLRSEDTAVYYCARGSYYYAMDYWGQGTLVTVSS |
| SEQ ID NO: | VH-2 | QVQLVQSGAEVKKPGASVKVSCKASGFKTEDTYMYWVRQAP |
| 1132 | | GQRLEWIGRIDPANGNTKYDPKFQDRATITADSSANTAYMELS |
| | | SLRSEDTAVYYCARGSYYYAMDYWGQGTLVTVSS |
| SEQ ID NO: | VH-3 | EVQLVESGGGLVQPGGSLKLSCAASGFKTEDTYMYWVRQAS |
| 1133 | | GKGLEWIGRIDPANGNTKYDPKFQDRATISADSSKNTAYLQM |
| | | INSLKTEDTAVYYCARGSYYYAMDYWGQGTLVTVSS |
| SEQ ID NO: | VH-4 | EVQLVQSGAEVKKPGESLRISCKASGFKTEDTYMYWVRQMPG |
| 1134 | | KGLEWIGRIDPANGNTKYDPKFQDQATISADSSINTAYLQWSS |
| | | ILKASDTAMYYCARGSYYYAMDYWGQGTLVTVSS |
| SEQ ID NO: | VH-5 | QVQLVQSGSELKKPGASVKVSCKASGFKTEDTYMYWVRQAP |
| 1135 | | GQGLEWIGRIDPANGNTKYDPKFQDRAVISADSSVNTAYLQIS |
| | | SLKAEDTAVYYCARGSYYYAMDYWGQGTLVTVSS |

VL for CAS1.1.3 (humanized) also referred to as Antibody H-H (humanized)
Binds to human TCRVβ 27

| | | |
|---|---|---|
| SEQ ID NO: | VL-1 | DIVLTQSPDSLAVSLGERATINCRASESVDSYGNSFMHWYQQK |
| 1136 | | PGQPPKLLIYRASNLESGVPDRFSGSGSRTDFTLTISSLQAEDVA |
| | | VYYCQQSNEDPYTFGQGTKLEIK |
| SEQ ID NO: | VL-2 | EIVLTQSPATLSLSPGERATLSCRASESVDSYGNSFMHWYQQK |
| 1137 | | PGQAPKLLIYRASNLESGIPARFSGSGSRTDFTLTISRLEPEDFA |
| | | VYYCQQSNEDPYTFGQGTKLEIK |
| SEQ ID NO: | VL-3 | DIQLTQSPSSLSASVGDRVTITCRASESVDSYGNSFMHWYQQK |
| 1138 | | PGQAPKLLIYRASNLESGVPSRFSGSGSRTDFTLTISSLQPEDVA |
| | | TYYCQQSNEDPYTFGQGTKLEIK |
| SEQ ID NO: | VL-4 | AIQLTQSPSSLSASVGDRVTITCRASESVDSYGNSFMHWYQQK |
| 1139 | | PGKAPKLLIYRASNLESGVPSRFSGSGSRTDFTLTISSLQPEDFA |
| | | TYYCQQSNEDPYTFGQGTKLEIK |
| SEQ ID NO: | VL-5 | EIVLTQSPDFQSVTPKEKVTITCRASESVDSYGNSFMHWYQQK |
| 1140 | | PDQSPKLLIYRASNLESGVPSRFSGSGSRTDFTLTINSLEAEDAA |
| | | TYYCQQSNEDPYTFGQGTKLEIK |

IMMU222 (murine) also referred to as BJ1461; or Antibody I
Binds to human TCRVβ 6-5,6-6,6-9

| | | |
|---|---|---|
| SEQ ID NO: | HC CDR1 (Kabat) | SYAMS |
| 1141 | | |
| SEQ ID NO: | HC CDR2 (Kabat) | HISNGGDYIYYADTVKG |
| 1142 | | |
| SEQ ID NO: | HC CDR3 (Kabat) | PSYYSDPWFFDV |
| 1143 | | |
| SEQ ID NO: | HC CDR1 (Chothia) | GFTFRSY |
| 1144 | | |
| SEQ ID NO: | HC CDR2 (Chothia) | SNGGDY |
| 1145 | | |
| SEQ ID NO: | HC CDR3 (Chothia) | PSYYSDPWFFDV |
| 1143 | | |
| SEQ ID NO: | HC CDR1 (Combined) | GFTFRSYAMS |
| 1146 | | |
| SEQ ID NO: | HC CDR2 (Combined) | HISNGGDYIYYADTVKG |
| 1142 | | |
| SEQ ID NO: | HC CDR3 (Combined) | PSYYSDPWFFDV |
| 1143 | | |
| SEQ ID NO: | LC CDR1 (Kabat) | SAGSSVSFMH |
| 1147 | | |
| SEQ ID NO: | LC CDR2 (Kabat) | DTSKLAS |
| 1148 | | |
| SEQ ID NO: | LC CDR3 (Kabat) | LQGSGFPLT |
| 1149 | | |
| SEQ ID NO: | LC CDR1 (Chothia) | GSSVSF |
| 1150 | | |
| SEQ ID NO: | LC CDR2 (Chothia) | DTSKLAS |
| 1148 | | |
| SEQ ID NO: | LC CDR3 (Chothia) | LQGSGFPLT |
| 1149 | | |
| SEQ ID NO: | LC CDR1 (Combined) | SAGSSVSFMH |
| 1147 | | |
| SEQ ID NO: | LC CDR2 (Combined) | DTSKLAS |
| 1148 | | |
| SEQ ID NO: | LC CDR3 (Combined) | LQGSGFPLT |
| 1149 | | |
| SEQ ID NO: | VL | ENVLTQSPAIMSASPGEKVTMTCSAGSSVSFMHWYQQKSSTSP |
| 1151 | | KLWIYDTSKLASGVPGRFSGSGSGNSFSLTISSMEAEDVAIYYC |
| | | LQGSGFPLTFGSGTKLEIK |
| SEQ ID NO: | VH | DVKLVESGEGLVKPGGSLKLSCAASGFTFRSYAMSWVRQTPE |
| 1152 | | KRLEWVAHISNGGDYIYYADTVKGRFTISRDNARNTLYLQMS |
| | | SLKSEDTAMYYCTRPSYYSDPWFFDVWGTGTTVTVSS |

VH for IMMU222 (humanized) also referred to as Antibody I-H
Binds to human TCRVβ 6-5,6-6,6-9

| | | |
|---|---|---|
| SEQ ID NO: | VH-1 | EVQLVESGGGLVQPGGSLRLSCAASGFTFRSYAMSWVRQAPG |
| 1153 | | KGLEWVAHISNGGDYIYYADTVKGRFTISRDNAKNSLYLQMN |
| | | SLRAEDTAVYYCTRPSYYSDPWFFDVWGQGTTVTVSS |
| SEQ ID NO: | VH-2 | QVQLVESGGGVVQPQRSLRLSCAASGFTFRSYAMSWVRQAPG |
| 1154 | | KGLEWVAHISNGGDYIYYADTVKGRFTISRDNSKNTLYLQMSS |
| | | LRAEDTAVYYCTRPSYYSDPWFFDVWGQGTTVTVSS |
| SEQ ID NO: | VH-3 | EVQLVESGGGLVQPGGSLRLSCAASGFTFRSYAMSWVRQAPG |
| 1155 | | KGLEWVAHISNGGDYIYYADTVKGRFTISRDNSKNTLYLQMN |
| | | SLRAEDTAVYYCTRPSYYSDPWFFDVWGQGTTVTVSS |
| SEQ ID NO: | VH-4 | QVQLVQSGSELKKPGASVKVSCKASGFTFRSYAMSWVRQAPG |
| 1156 | | QGLEWVAHISNGGDYIYYADTVKGRFVISRDNSVNTLYLQISS |
| | | LKAEDTAVYYCTRPSYYSDPWFFDVWGQGTTVTVSS |
| SEQ ID NO: | VH-5 | QVQLVQSGAEVKKPGASVKVSCKASGFTFRSYAMSWVRQAP |
| 1157 | | GQRLEWVAHISNGGDYIYYADTVKGRFTITRDNSANTLYMEL |
| | | SSLRSEDTAVYYCTRPSYYSDPWFFDVWGQGTTVTVSS |

VL for IMMU222 (humanized)) also referred to as Antibody I-H
Binds to human TCRVβ 6-5,6-6,6-9

| | | |
|---|---|---|
| SEQ ID NO: | VL-1 | ENVLTQSPATLSLSPGERATLSCSAGSSVSFMHWYQQKPGQAP |
| 1158 | | KLLIYDTSKLASGIPARFSGSGSGNDFTLTISSLEPEDFAVYYCL |
| | | QGSGFPLTFGQGTKLEIK |
| SEQ ID NO: | VL-2 | ENVLTQSPDFQSVTPKEKVTITCSAGSSVSFMHWYQQKPDQSP |
| 1159 | | KLLIYDTSKLASGVPSRFSGSGSGNDFTLTINSLEAEDAATYYC |
| | | LQGSGFPLTFGQGTKLEIK |
| SEQ ID NO: | VL-3 | DNQLTQSPSSLSASVGDRVTITCSAGSSVSFMHWYQQKPGKVP |
| 1160 | | KLLIYDTSKLASGVPSRFSGSGSGNDFTLTISSLQPEDVATYYCL |
| | | QGSGFPLTFGQGTKLEIK |
| SEQ ID NO: | VL-4 | ANQLTQSPSSLSASVGDRVTITCSAGSSVSFMHWYQQKPGKAP |
| 1161 | | KLLIYDTSKLASGVPSRFSGSGSGNDFTLTISSLQPEDFATYYCL |
| | | QGSGFPLTFGQGTKLEIK |
| SEQ ID NO: | VL-5 | DNVLTQSPDSLAVSLGERATINCSAGSSVSFMHWYQQKPGQPP |
| 1162 | | KLLIYDTSKLASGVPDRFSGSGSGNDFTLTISSLQAEDVAVYYC |
| | | LQGSQFPLTFGQGTKLEIK |

REA1062 (murine), also referred to as BJM1108, BJ1189 or as Antibody J
Binds to human TCRVβ 5-1

| | | |
|---|---|---|
| SEQ ID NO: | HC CDR1 (Kabat) | DYNIH |
| 1163 | | |
| SEQ ID NO: | HC CDR2 (Kabat) | YINPYNGRTGYNQKFKA |
| 1164 | | |
| SEQ ID NO: | HC CDR3 (Kabat) | WDGSSYFDY |
| 1165 | | |
| SEQ ID NO: | HC CDR1 (Chothia) | GYTFTDYNIH |
| 1166 | | |
| SEQ ID NO: | HC CDR2 (Chothia) | NPYNGR |
| 1167 | | |
| SEQ ID NO: | HC CDR3 (Chothia) | WDGSSYFDY |
| 1165 | | |
| SEQ ID NO: | HC CDR1 (Combined) | GYTFTDYNIH |
| 1166 | | |
| SEQ ID NO: | HC CDR2 (Combined) | YINPYNGRTGYNQKFKA |
| 1164 | | |
| SEQ ID NO: | HC CDR3 (Combined) | WDGSSYFDY |
| 1165 | | |
| SEQ ID NO: | LC CDR1 (Kabat) | SASSSVSYMH |
| 1168 | | |
| SEQ ID NO: | LC CDR2 (Kabat) | EISKLAS |
| 1169 | | |
| SEQ ID NO: | LC CDR3 (Kabat) | QQWNYPLLT |
| 1170 | | |
| SEQ ID NO: | LC CDR1 (Chothia) | SSSVSY |
| 1297 | | |
| SEQ ID NO: | LC CDR2 (Chothia) | EISKLAS |
| 1169 | | |
| SEQ ID NO: | LC CDR3 (Chothia) | QQWNYPLLT |
| 1170 | | |
| SEQ ID NO: | LC CDR1 (Combined) | SASSSVSYMH |
| 1168 | | |
| SEQ ID NO: | LC CDR2 (Combined) | EISKLAS |
| 1169 | | |
| SEQ ID NO: | LC CDR3 (Combined) | QQWNYPLLT |
| 1170 | | |
| SEQ ID NO: | VL | EIVLTQSPAITAASLGQKVTITCSASSSVSYMHWYQQKSGTSPK |
| 1171 | | PWIYEISKLASGVPARFSGSGSGTSYSLTISSMEAEDAAIYYCQ |
| | | QWNYPLLTFGAGTKLELK |
| SEQ ID NO: | VH | EVQLQQSGPVLVKPGASVRMSCKASGYTFTDYNIHWVKQSHG |
| 1172 | | RSLEWVGYINPYNGRTGYNQKFKAKATLTVDKSSSTAYMDLR |
| | | SLTSEDSAVYYCARWDGSSYFDYWGQGTTLTVSS |

VH for REA1062 (humanized) also referred to as Antibody J-H
Binds to human TCRVβ 5-1

| | | |
|---|---|---|
| SEQ ID NO: | VH-1 | QVQLVQSGAEVKKPGSSVKVSCKASGYTFTDYNIHWVRQAPG |
| 1173 | | QGLEWVGYINPYNGRTGYNQKFKARATLTVDKSTSTAYMELS |
| | | SLRSEDTAVYYCARWDGSSYFDYWGQGTTVTVSS |
| SEQ ID NO: | VH-2 | QVQLVQSGAEVKKPGASVKVSCKASGYTFTDYNIHWVRQAP |
| 1174 | | GQGLEWVGYINPYNGRTGYNQKFKARATLTVDKSTSTAYME |
| | | LRSLRSDDMAVYYCARWDQSSYFDYWGQGTTVTVSS |
| SEQ ID NO: | VH-3 | QVQLVQSGAEVKKPGASVKVSCKASGYTFTDYNIHWVRQAT |
| 1175 | | GQGLEWVGYINPYNGRTGYNQKFKARATLTVNKSISTAYMEL |
| | | SSLRSEDTAVYYCARWDGSSYFDYWGQGTTVTVSS |
| SEQ ID NO: | VH-4 | EVQLVESGGGLVQPGRSLRLSCTASGYTFTDYNIHWVRQAPG |
| 1176 | | KGLEWVGYINPYNGRTGYNQKFKARATLSVDKSKSIAYLQMN |
| | | SLKTEDTAVYYCARWDGSSYFDYWGQGTTVTVSS |
| SEQ ID NO: | VH-5 | QVQLVQSGSELKKPGASVKVSCKASGYTFTDYNIHWVRQAPG |
| 1177 | | QGLEWVGYINPYNGRTGYNQKFKARAVLSVDKSVSTAYLQIS |
| | | SLKAEDTAVYYCARWDGSSYFDYWGQGTTVTVSS |

VL for REA1062 (humanized) also referred to as Antibody J-H
Binds to human TCRVβ 5-1

| | | |
|---|---|---|
| SEQ ID NO: | VL-1 | EIVLTQSPATLSLSPGERATLSCSASSSVSYMHWYQQKPGQAP |
| 1178 | | KLLIYEISKLASGIPARFSGSGSGTDYTLTISSLEPEDFAVYYCQ |
| | | QWNYPLLTFGQGTKLEIK |
| SEQ ID NO: | VL-2 | EIVLTQSPATLSLSPGERATLSCSASSSVSYMHWYQQKPGQAP |
| 1179 | | KLLIYEISKLASGIPARFSGSGSGTDYTLTISRLEPEDFAVYYCQ |
| | | QWNYPLLTFGQGTKLEIK |
| SEQ ID NO: | VL-3 | EIVLTQSPDFQSVTPKEKVTITCSASSSVSYMHWYQQKPDQSPK |
| 1180 | | LLIYEISKLASGVPSRFSGSGSGTDYTLTINSLEAEDAATYYCQQ |
| | | WNYPLLTFGQGTKLEIK |
| SEQ ID NO: | VL-4 | DIQLTQSPSFLSASVGDRVTITCSASSSVSYMHWYQQKPGKAP |
| 1181 | | KLLIYEISKLASGVPSRFSGSGSGTEYTLTISSLQPEDFATYYCQ |
| | | QWNYPLLTFGQGTKLEIK |
| SEQ ID NO: | VL-5 | AIQLTQSPSSLSASVGDRVTITCSASSSVSYMHWYQQKPGKAP |
| 1182 | | KLLIYEISKLASGVPSRFSGSGSGTDYTLTISSLQPEDFATYYCQ |
| | | QWNYPLLTFGQGTKLEIK |
| SEQ ID NO: | VL-6 | AIRLTQSPFSLSASVGDRVTITCSASSSVSYMHWYQQKPAKAP |
| 1183 | | KLFIYEISKLASGVPSRFSGSGSGTDYTLTISSLQPEDFATYYCQ |
| | | QWNYPLLTFGQGTKLEIK |
| SEQ ID NO: | VL-7 | DIVLTQSPDSLAVSLGERATINCSASSSVSYMHWYQQKPGQPP |
| 1184 | | KLLIYEISKLASGVPDRFSGSGSGTDYTLTISSLQAEDVAVYYC |
| | | QQWNYPLLTFGQGTKLEIK |

JOVI-3 (murine), also referred to as BJ1187 or Antibody K
Binds to human TCRVβ 28

| | | |
|---|---|---|
| SEQ ID NO: | HC CDR1 (Kabat) | GSWMN |
| 1185 | | |
| SEQ ID NO: | HC CDR2 (Kabat) | RIYPGDGDTDYSGKFKG |
| 1186 | | |
| SEQ ID NO: | HC CDR3 (Kabat) | SGYFNYVPVFDY |
| 1187 | | |
| SEQ ID NO: | HC CDR1 (Chothia) | GYTFSGS |
| 1188 | | |
| SEQ ID NO: | HC CDR2 (Chothia) | YPGDGD |
| 1189 | | |
| SEQ ID NO: | HC CDR3 (Chothia) | SGYFNYVPVFDY |
| 1187 | | |
| SEQ ID NO: | HC CDR1 (Combined) | GYTFSGSWMN |
| 1190 | | |
| SEQ ID NO: | HC CDR2 (Combined) | RIYPGDGDTDYSGKFKG |
| 1186 | | |
| SEQ ID NO: | HC CDR3 (Combined) | SGYFNYVPVFDY |
| 1187 | | |
| SEQ ID NO: | LC CDR1 (Kabat) | SANSTVGYIH |
| 1191 | | |
| SEQ ID NO: | LC CDR2 (Kabat) | TTSNLAS |
| 1192 | | |
| SEQ ID NO: | LC CDR3 (Kabat) | HQWSFYPT |
| 1193 | | |
| SEQ ID NO: | LC CDR1 (Chothia) | NSTVGY |
| 1194 | | |
| SEQ ID NO: | LC CDR2 (Chothia) | TTSNLAS |
| 1192 | | |
| SEQ ID NO: | LC CDR3 (Chothia) | HQWSFYPT |
| 1193 | | |
| SEQ ID NO: | LC CDR1 (Combined) | SANSTVGYIH |
| 1191 | | |
| SEQ ID NO: | LC CDR2 (Combined) | TTSNLAS |
| 1192 | | |
| SEQ ID NO: | LC CDR3 (Combined) | HQWSFYPT |
| 1193 | | |
| SEQ ID NO: | VL | QIVLTQSPAIMSASLGEEIALTCSANSTVGYIHWYQQKSGTSPK |
| 1195 | | LLIYTTSNLASGVPSRFSGSGSGTFYSLTISSVEAEDAADYFCHQ |
| | | WSFYPTFGGGTKLEIK |
| SEQ ID NO: | VH | QIQLQQSGPEVVKPGASVQISCKASGYTFSGSWMNWVKQRPG |
| 1196 | | KGLEWIGRIYPGDGDTDYSGKFKGRATLTADKSSSTAYMRLSS |
| | | LTSEDSAVYFCARSGYFNYVPVFDYWGQGTTLSVSS |

VH for JOVI-3 (humanized) also referred to as Antibody K-H
Binds to human TCRVβ 28

| | | |
|---|---|---|
| SEQ ID NO: | VH-1 | QIQLVQSGAEVKKPGASVKVSCKASGYTFSGSWMNWVRQAP |
| 1197 | | GQGLEWIGRIYPGDGDTDYSGKFKGRATLTADKSTSTAYMEL |
| | | SSLRSEDTAVYYCARSGYFNYVPVFDYWGQGTTVTVSS |
| SEQ ID NO: | VH-2 | QIQLVQSGAEVKKPGSSVKVSCKASGYTFSGSWMNWVRQAP |
| 1198 | | GQGLEWIGRIYPGDGDTDYSGKFKGRATLTADKSTSTAYMEL |
| | | SSLRSEDTAVYYCARSGYFNYVPVFDYWGQGTTVTVSS |
| SEQ ID NO: | VH-3 | EIQLVQSGAEVKKPGESLKISCKASGYTFSGSWMNWVRQMPG |
| 1199 | | KGLEWIGRIYPGDGDTDYSGKFKGQATLSADKSISTAYLQWSS |
| | | LKASDTAMYYCARSGYFNYVPVFDYWGQGTTVTVSS |
| SEQ ID NO: | VH-4 | QIQLVQSGSELKKPGASVKVSCKASGYTFSGSWMNWVRQAPG |
| 1200 | | QGLEWIGRIYPGDGDTDYSGKFKGRAVLSADKSVSTAYLQISS |
| | | LKAEDTAVYYCARSGYFNYVPVFDYWGQGTTVTVSS |
| SEQ ID NO: | VH-5 | QIQLVQSGSELKKPGASVKVSCKASGYTFSGSWMNWVRQAPG |
| 1201 | | QGLEWIGRIYPGDGDTDYSGKFKGRAVLSADKSVSMAYLQISS |
| | | LKAEDTAVYYCARSGYFNYVPVFDYWGQGTTVTVSS |
| SEQ ID NO: | VH-6 | EIQLVESGGGLVQPQRSLRLSCTASGYTFSGSWMNWVRQAPG |
| 1202 | | KGLEWIGRIYPGDGDTDYSGKFKGRATLSADKSKSIAYLQMNS |
| | | ILKTEDTAVYYCARSGYFNYVPVFDYWGQGTTVTVSS |

VL for JOVI-3 (humanized) also referred to as Antibody K-H
Binds to human TCRVβ 28

| | | |
|---|---|---|
| SEQ ID NO: | VL-1 | EIVLTQSPATLSLSPGERATLSCSANSTVGYIHWYQQKPGQAPK |
| 1203 | | LLIYTTSNLASGIPARFSGSGSGTDYTLTISSLEPEDFAVYFCHQ |
| | | WSFYPTFGQGTKLEIK |
| SEQ ID NO: | VL-2 | DIQLTQSPSFLSASVGDRVTITCSANSTVGYIHWYQQKPGKAPK |
| 1204 | | LLIYTTSNLASGVPSRFSGSGSGTEYTLTISSLQPEDFATYFCHQ |
| | | WSFYPTFGQGTKLEIK |
| SEQ ID NO: | VL-3 | EIVLTQSPATLSLSPGERATLSCSANSTVGYIHWYQQKPGQAPK |
| 1205 | | LLIYTTSNLASGIPARFSGSGPGTDYTLTISSLEPEDFAVYFCHQ |
| | | WSFYPTFGQGTKLEIK |
| SEQ ID NO: | VL-4 | DIVLTQSPDSLAVSLGERATINCSANSTVGYIHWYQQKPGQPP |
| 1206 | | KLLIYTTSNLASGVPDRFSGSGSGTDYTLTISSLQAEDVAVYFC |
| | | HQWSFYPTFGQGTKLEIK |
| SEQ ID NO: | VL-5 | EIVLTQSPDFQSVTPKEKVTITCSANSTVGYIHWYQQKPDQSPK |
| 1207 | | LLIYTTSNLASGVPSRFSGSGSGTDYTLTINSLEAEDAATYFCH |
| | | QWSFYPTFGQGTKLEIK |

ZOE (murine), also referred to as BJ1538 or as Antibody L
Binds to human TCRVβ 4-1,4-2,4-3

| | | |
|---|---|---|
| SEQ ID NO: | HC CDR1 (Kabat) | DYYMY |
| 1208 | | |
| SEQ ID NO: | HC CDR2 (Kabat) | TISGGGSYTYSPDSVKG |
| 1209 | | |
| SEQ ID NO: | HC CDR3 (Kabat) | ERDIYYGNFNAMVY |
| 1210 | | |
| SEQ ID NO: | HC CDR1 (Chothia) | GFTFSDY |
| 1211 | | |
| SEQ ID NO: | HC CDR2 (Chothia) | SGGQSY |
| 1212 | | |
| SEQ ID NO: | HC CDR3 (Chothia) | ERDIYYGNFNAMVY |
| 1210 | | |
| SEQ ID NO: | HC CDR1 (Combined) | GFTFSDYYMY |
| 1213 | | |
| SEQ ID NO: | HC CDR2 (Combined) | TISGGGSYTYSPDSVKG |
| 1209 | | |
| SEQ ID NO: | HC CDR3 (Combined) | ERDIYYGNFNAMVY |
| 1210 | | |
| SEQ ID NO: | LC CDR1 (Kabat) | RASKSVSTSGYSYMH |
| 1214 | | |
| SEQ ID NO: | LC CDR2 (Kabat) | LASNLES |
| 1215 | | |
| SEQ ID NO: | LC CDR3 (Kabat) | QHSRDLPWT |
| 1216 | | |
| SEQ ID NO: | LC CDR1 (Chothia) | SKSVSTSGYSY |
| 1217 | | |
| SEQ ID NO: | LC CDR2 (Chothia) | LASNLES |
| 1215 | | |
| SEQ ID NO: | LC CDR3 (Chothia) | QHSRDLPWT |
| 1216 | | |
| SEQ ID NO: | LC CDR1 (Combined) | RASKSVSTSGYSYMH |
| 1214 | | |
| SEQ ID NO: | LC CDR2 (Combined) | LASNLES |
| 1215 | | |
| SEQ ID NO: | LC CDR3 (Combined) | QHSRDLPWT |
| 1216 | | |
| SEQ ID NO: | VL | DIVLTQSPVSLTVSLGQRATISCRASKSVSTSGYSYMHWYQQK |
| 1218 | | PGQPPKLLIYLASNLESGVPARFSGSGSGTDFTLNIHPVEEEDA |
| | | ATYYCQHSRDLPWTFGGGTKLEIK |
| SEQ ID NO: | VH | EVQLVESGGGLVKPGGSLKLSCAASGFTFSDYYMYWVRQTPE |
| 1219 | | KRLEWVATISGGGSYTYSPDSVKGRFTISRDNAKNNLYLQMSS |
| | | LRSEDTAMYFCARERDIYYGNFNAMVYWGRGTSVTVSS |

VH for ZOE (humanized) also referred to as Antibody L-H
Binds to human TCRVβ 4-1,4-2,4-3

| | | |
|---|---|---|
| SEQ ID NO: | VH-1 | EVQLLESGGGLVQPGGSLRLSCAASGFTFSDYYMYWVRQAPG |
| 1220 | | KGLEWVATISGGGSYTYSPDSVKGRFTISRDNSKNTLYLQMNS |
| | | LRAEDTAVYYCARERDIYYGNFNAMVYWGRQTLVTVSS |
| SEQ ID NO: | VH-2 | EVQLVESGGGLVQPGGSLRLSCAASGFTFSDYYMYWVRQAPG |
| 1221 | | KGLEWVATISGGGSYTYSPDSVKQRFTISRDNAKNSLYLQMNS |
| | | LRAEDTAVYYCARERDIYYGNFNAMVYWGRGTLVTVSS |
| SEQ ID NO: | VH-3 | QVQLVESGGGVVQPGRSLRLSCAASGFTFSDYYMYWVRQAP |
| 1222 | | GKGLEWVATISGGGSYTYSPDSVKGRFTISRDNSKNTLYLQM |
| | | NSLRAEDTAVYYCARERDIYYGNFNAMVYWGRGTLVTVSS |
| SEQ ID NO: | VH-4 | QVQLVESGGGLVKPGGSLRLSCAASGFTFSDYYMYWIRQAPG |
| 1223 | | KGLEWVATISGGGSYTYSPDSVKGRFTISRDNAKNSLYLQMNS |
| | | ILRAEDTAVYYCARERDIYYGNFNAMVYWGRGTLVTVSS |

VL for ZOE (humanized) also referred to as Antibody L-H
Binds to human TCRVβ 4-1,4-2,4-3

| | | |
|---|---|---|
| SEQ ID NO: | VL-1 | EIVLTQSPGTLSLSPGERATLSCRASKSVSTSGYSYMHWYQQK |
| 1224 | | PGQAPRLLIYLASNLESGIPDRFSGSGSGTDFTLTISRLEPEDFAV |
| | | YYCQHSRDLPWTFGGGTKVEIK |
| SEQ ID NO: | VL-2 | EIVLTQSPATLSLSPGERATLSCRASKSVSTSGYSYMHWYQQK |
| 1225 | | PGQAPRLLIYLASNLESGIPARFSGSGSGTDFTLTISSLEPEDFAV |
| | | YYCQHSRDLPWTFGGGTKVEIK |
| SEQ ID NO: | VL-3 | DIQLTQSPSTLSASVGDRVTITCRASKSVSTSGYSYMHWYQQK |
| 1226 | | PGKAPKLLIYLASNLESGVPSRFSGSGSGTEFTLTISSLQPDDFA |
| | | TYYCQHSRDLPWTFGGGTKVEIK |
| SEQ ID NO: | VL-4 | AIQLTQSPSSLSASVGDRVTITCRASKSVSTSGYSYMHWYQQK |
| 1227 | | PGKAPKLLIYLASNLESGVPSRFSGSGSGTDFTLTISSLQPEDFA |
| | | TYYCQHSRDLPWTFGGGTKVEIK |

Anti-TCRvb19 (murine), also referred to as BJ1465; or Antibody M
Binds to human TCRVβ 19

| | | |
|---|---|---|
| SEQ ID NO: | HC CDR1 (Kabat) | GYFWN |
| 1229 | | |
| SEQ ID NO: | HC CDR2 (Kabat) | YISYDGSNNYNPSLKN |
| 1230 | | |
| SEQ ID NO: | HC CDR3 (Kabat) | PSPGTGYAVDY |
| 1231 | | |
| SEQ ID NO: | HC CDR1 (Chothia) | GYSITSGY |
| 1232 | | |
| SEQ ID NO: | HC CDR2 (Chothia) | SYDGSN |
| 1233 | | |
| SEQ ID NO: | HC CDR3 (Chothia) | PSPGTGYAVDY |
| 1231 | | |
| SEQ ID NO: | HC CDR1 (Combined) | GYSITSGYFWN |
| 1234 | | |
| SEQ ID NO: | HC CDR2 (Combined) | YISYDGSNNYNPSLKN |
| 1230 | | |
| SEQ ID NO: | HC CDR3 (Combined) | PSPGTGYAVDY |
| 1231 | | |
| SEQ ID NO: | LC CDR1 (Kabat) | RSSQSLVHSNQNTYLH |
| 1235 | | |
| SEQ ID NO: | LC CDR2 (Kabat) | KVSNRFS |
| 1236 | | |
| SEQ ID NO: | LC CDR3 (Kabat) | SQSTHVPFT |
| 1237 | | |
| SEQ ID NO: | LC CDR1 (Chothia) | SQSLVHSNGNTY |
| 1238 | | |
| SEQ ID NO: | LC CDR2 (Chothia) | KVSNRFS |
| 1236 | | |
| SEQ ID NO: | LC CDR3 (Chothia) | SQSTHVPFT |
| 1237 | | |
| SEQ ID NO: | LC CDR1 (Combined) | RSSQSLVHSNGNTYLH |
| 1235 | | |
| SEQ ID NO: | LC CDR2 (Combined) | KVSNRFS |
| 1236 | | |
| SEQ ID NO: | LC CDR3 (Combined) | SQSTHVPFT |
| 1237 | | |
| SEQ ID NO: | VL | NVVMTQTPLSLPVSLGDQASISCRSSQSLVHSNGNTYLHWYLQ |
| 1239 | | KPGQSPKFLIYKVSNRFSGVPDRFSGGGSGTEFTLKISRVEAED |
| | | LGVYFCSQSTHVPFTFGSGTKLEIK |
| SEQ ID NO: | VH | NVQLQESGPGLVKPSQSLSLTCSVAGYSITSGYFWNWIRQFPG |
| 1240 | | NKLEWMGYISYDGSNNYNPSLKNRISITRDTSKNQFFLKLNSV |
| | | TTEDTATYYCASPSPGTGYAVDYWGQGTSVTVSS |

VH for Anti-TCRvb19 (humanized) also referred to as Antibody M-H
Binds to human TCRVβ 19

| | | |
|---|---|---|
| SEQ ID NO: | VH-1 | QVQLQESGPGLVKPSETLSLTCTVSGYSITSGYFWNWIRQPPGK |
| 1241 | | GLEWIGYISYDGSNNYNPSLKNRVTISRDTSKNQFSLKLSSVTA |
| | | ADTAVYYCASPSPGTGYAVDYWGQGTLVTVSS |
| SEQ ID NO: | VH-2 | QVQLQESGPGLVKPSETLSLTCTVSGYSITSGYFWNWIRQPPGK |
| 1242 | | GLEWIGYISYDGSNNYNPSLKNRVTISRDTSKNQFSLKLSSVTA |
| | | ADTAVYYCASPSPGTGYAVDYWGQGTLVTVSS |
| SEQ ID NO: | VH-3 | QVQLVESGGGLVQPGGSLRLSCSVSGYSITSGYFWNWVRQAP |
| 1243 | | GKGLEWVGYISYDGSNNYNPSLKNRFTISRDTSKNTFYLQMNS |
| | | LRAEDTAVYYCASPSPGTGYAVDYWGQGTLVTVSS |

VL for Anti-TCRvb19 (humanized) also referred to as Antibody M-H
Binds to human TCRVβ 19

| | | |
|---|---|---|
| SEQ ID NO: | VL-1 | VVMTQSPGTLSLSPGERATLSCRSSQSLVHSNQNTYLHWYQQ |
| 1244 | | KPGQAPRFLIYKVSNRFSGIPDRFSGSGSGTDFTLTISRLEPEDF |
| | | AVYFCSQSTHVPFTFGQGTKLEIK |
| SEQ ID NO: | VL-2 | EVVMTQSPATLSLSPGERATLSCRSSQSLVHSNGNTYLHWYQQ |
| 1245 | | KPGQAPRFLIYKVSNRFSGIPARFSGSGSGTDFTLTISSLEPEDFA |
| | | VYFCSQSTHVPFTFGQGTKLEIK |
| SEQ ID NO: | VL-3 | EVVMTQSPATLSVSPGERATLSCRSSQSLVHSNGNTYLHWYQ |
| 1246 | | QKPGQAPRFLIYKVSNRFSGIPARFSGSGSGTEFTLTISSLQSEDF |
| | | AVYFCSQSTHVPFTFGQGTKLEIK |
| SEQ ID NO: | VL-4 | DVQMTQSPSSLSASVGDRVTITCRSSQSLVHSNGNTYLHWYQ |
| 1247 | | QKPGKAPKFLIYKVSNRFSGVPSRFSGSGSGTDFTFTISSLQPED |
| | | IATYFCSQSTHVPFTFGQGTKLEIK |

BL37.2 (murine), also referred to as BJ1539 or Antibody N
Binds to human TCRVβ 9

| | | |
|---|---|---|
| SEQ ID NO: | HC CDR1 (Kabat) | DYIVH |
| 1248 | | |
| SEQ ID NO: | HC CDR2 (Kabat) | WINTYTGTPTYADDFEG |
| 1249 | | |
| SEQ ID NO: | HC CDR3 (Kabat) | SWRRGIRGIGFDY |
| 1250 | | |
| SEQ ID NO: | HC CDR1 (Chothia) | GYTFTDY |
| 1251 | | |
| SEQ ID NO: | HC CDR2 (Chothia) | NTYTGT |
| 1252 | | |
| SEQ ID NO: | HC CDR3 (Chothia) | SWRRGIRGIGFDY |
| 1250 | | |
| SEQ ID NO: | HC CDR1 (Combined) | GYTFTDYIVH |
| 1253 | | |
| SEQ ID NO: | HC CDR2 (Combined) | WINTYTGTPTYADDFEG |
| 1249 | | |
| SEQ ID NO: | HC CDR3 (Combined) | SWRRGIRGIGFDY |
| 1250 | | |
| SEQ ID NO: | LC CDR1 (Kabat) | KASKSINKYLA |
| 2254 | | |
| SEQ ID NO: | LC CDR2 (Kabat) | DGSTLQS |
| 1255 | | |
| SEQ ID NO: | LC CDR3 (Kabat) | QQHNEYPPT |
| 1256 | | |
| SEQ ID NO: | LC CDR1 (Chothia) | SKSINKY |
| 1257 | | |
| SEQ ID NO: | LC CDR2 (Chothia) | DGSTLQS |
| 1255 | | |
| SEQ ID NO: | LC CDR3 (Chothia) | QQHNEYPPT |
| 1256 | | |
| SEQ ID NO: | LC CDR1 (Combined) | KASKSINKYLA |
| 1254 | | |
| SEQ ID NO: | LC CDR2 (Combined) | DGSTLQS |
| 1255 | | |
| SEQ ID NO: | LC CDR3 (Combined) | QQHNEYPPT |
| 1256 | | |
| SEQ ID NO: | VL | DVQMTQSPYNLAASPGESVSINCKASKSINKYLAWYQQKPGK |
| 1258 | | PNKLLIYDGSTLQSGIPSRFSGSGSGTDFTLTIRGLEPEDFGLYY |
| | | CQQHNEYPPTFGAGTKLELK |
| SEQ ID NO: | VH | QLQLVQSGPELREPGESVKISCKASGYTFTDYIVHWVKQAPGK |
| 1259 | | GLKWMGWINTYTGTPTYADDFEGRFVFSLEASASTANLQISNL |
| | | KNEDTATYFCARSWRRGIRGIGFDYWGQGVMVTVSS |

VH for BL37.2 (humanized) also referred to as Antibody N-H
Binds to human TCRVβ 9

| | | |
|---|---|---|
| SEQ ID NO: | VH-1 | QLQLVQSGAEVKKPGASVKVSCKASGYTFTDYIVHWVRQAPG |
| 1260 | | QGLEWMGWINTYTGTPTYADDFEGWVTMTLDASISTAYMEL |
| | | SRLRSDDTAVYYCARSWRRGIRGIGFDYWGQGTMVTVSS |
| SEQ ID NO: | VH-2 | QLQLVQSGAEVKKPGASVKVSCKASGYTFTDYIVHWVRQAPG |
| 1261 | | QGLEWMGWINTYTGTPTYADDFEGRVTMTLDASTSTAYMEL |
| | | SSLRSEDTAVYYCARSWRRGIRGIGFDYWGQGTMVTVSS |
| SEQ ID NO: | VH-3 | QLQLVQSGAEVKKPGASVKVSCKASGYTFTDYIVHWVRQAPG |
| 1262 | | QRLEWMGWINTYTGTPTYADDFEGRVTITLDASASTAYMELS |
| | | SLRSEDMAVYYCARSWRRGIRGIGFDYWGQGTMVTVSS |
| SEQ ID NO: | VH-4 | QLQLVQSGAEVKKPGASVKVSCKASGYTFTDYIVHWVRQATG |
| 1263 | | QGLEWMGWINTYTGTPTYADDFEGRVTMTLNASISTAYMELS |
| | | SLRSEDTAVYYCARSWRRGIRGIGFDYWGQGTMVTVSS |

VL for BL37.2 (humanized) also referred to as Antibody N-H
Binds to human TCRVβ 9

| | | |
|---|---|---|
| SEQ ID NO: | VL-1 | EVVMTQSPGTLSLSPGERATLSCKASKSINKYLAWYQQKPGQ |
| 1264 | | APRLLIYDGSTLQSGIPDRFSGSGSGTDFTLTISRLEPEDFAVYY |
| | | CQQHNEYPPTFGQGTKLEIK |
| SEQ ID NO: | VL-2 | EVVMTQSPATLSLSPGERATLSCKASKSINKYLAWYQQKPGQ |
| 1265 | | APRLLIYDGSTLQSGIPARFSGSGSGTDFTLTISSLEPEDFAVYY |
| | | CQQHNEYPPTFGQGTKLEIK |
| SEQ ID NO: | VL-3 | DVQMTQSPSSLSASVGDRVTITCKASKSINKYLAWYQQKPGK |
| 1266 | | APKLLIYDGSTLQSGVPSRFSGSGSGTDFTLTISSLQPEDFATYY |
| | | CQQHNEYPPTFGQGTKLEIK |
| SEQ ID NO: | VL-4 | AVRMTQSPSSFSASTGDRVTITCKASKSINKYLAWYQQKPGKA |
| 1267 | | PKLLIYDGSTLQSGVPSRFSGSGSGTDFTLTISCLQSEDFATYYC |
| | | QQHNEYPPTFGQGTKLEIK |

IG125 (murine) binds to TRVβ 11-2; also referred to as Antibody O

| | | |
|---|---|---|
| SEQ ID NO: | HC CDR1 (Kabat) | NYGVH |
| 1268 | | |
| SEQ ID NO: | HC CDR2 (Kabat) | VIWSDGSTDYDTAFIS |
| 1269 | | |
| SEQ ID NO: | HC CDR3 (Kabat) | RAVVADFDY |
| 1270 | | |
| SEQ ID NO: | HC CDR1 (Chothia) | GFSLTN |
| 1271 | | |
| SEQ ID NO: | HC CDR2 (Chothia) | VIWSDGSTD |
| 1272 | | |
| SEQ ID NO: | HC CDR3 (Chothia) | RAVVADFDY |
| 1270 | | |
| SEQ ID NO: | HC CDR1 (combined) | GFSLTNYGVH |
| 1273 | | |
| SEQ ID NO: | HC CDR2 (combined) | VIWSDGSTDYDTAFIS |
| 1269 | | |
| SEQ ID NO: | HC CDR3 (combined) | RAVVADFDY |
| 1270 | | |
| SEQ ID NO: | VH | QVQLKQSGPGLLQPSQSLSITCTVSGFSLINYGVHWVRQSPGK |
| 1274 | | GLEWLGVIWSDGSTDYDTAFISRLSISKDNSKSQVFFKLNSLQA |
| | | DDTAIYYCARRAVVADFDYWGQGTTLTVSS |
| SEQ ID NO: | LC CDR1 (Kabat) | KASKEVTIFGSISALH |
| 1275 | | |
| SEQ ID NO: | LC CDR2 (Kabat) | NGAKLES |
| 1276 | | |
| SEQ ID NO: | LC CDR3 (Kabat) | LQNKEVPFT |
| 1277 | | |
| SEQ ID NO: | LC CDR1 (Chothia) | KASKEVTIFGSISALH |
| 1275 | | |
| SEQ ID NO: | LC CDR2 (Chothia) | NGAKLES |
| 1276 | | |
| SEQ ID NO: | LC CDR3 (Chothia) | LQNKEVPFT |
| 1277 | | |
| SEQ ID NO: | LC CDR1 (combined) | KASKEVTIFGSISALH |
| 1275 | | |
| SEQ ID NO: | LC CDR2 (combined) | NGAKLES |
| 1276 | | |
| SEQ ID NO: | LC CDR3 (combined) | LQNKEVPFT |
| 1277 | | |
| SEQ ID NO: | VL | DIVLTQSPASLAVSLGQKATISCKASKEVTIFGSISALHWYQQ |
| 1278 | | KPGQPPKLIYNGAKLESGVSARFSDSGSQNRSPFGNQLSFTLTI |
| | | APVEADDAATYYCLQNKEVPFTFQSGTKLEIK |

VL for IG125 (humanized) also referred to as Antibody O-H
binds to TRVβ 11-2

| | | |
|---|---|---|
| SEQ ID NO: | VL-1 | DIVLTQSPDSLAVSLGERATINCKASKEVTIFGSISALHWYQQ |
| 1279 | | KPGQPPKLLYNGAKLESGVSARFGVPDRFSRSGSGLDFTLTIS |
| | | SLQAEDVAVYYCLQNKEVPFTFGQGTKLEIK |
| SEQ ID NO: | VL-2 | EIVLTQSPDFQSVTPKEKVTITCKASKEVTIFQSISALHWYQQK |
| 1280 | | PDQSPKLLYNGAKLESGVSARFGVPSRFSRSGSGLDFTLTINSL |
| | | EAEDAATYYCLQNKEVPFTFGQGTKLEIK |
| SEQ ID NO: | VL-3 | AIQLTQSPSSLSASVGDRVTITCKASKEVTIFGSISALHWYQQK |
| 1281 | | PGKAPKLLYNGAKLESGVSARFGVPSRFSRSGSGLDFTLTISSL |
| | | QPEDFATYYCLQNKEVPFTFGQGTKLEIK |
| SEQ ID NO: | VL-4 | DIVLTQTPLSLSVTPGQPASISCKASKEVTIFGSISALHWYLQK |
| 1282 | | PGQPPKLLYNGAKLESGVSARFGVPDRFSRSGSGLDFTLKISR |
| | | VEAEDVGVYYCLQNKEVPFTFGQGTKLEIK |

VH for IG125 (humanized) also referred to as Antibody O-H
binds to TRVβ 11-2

| | | |
|---|---|---|
| SEQ ID NO: | VH-1 | QVTLKESGPVLVKPTETLTLTCTVSGFSLTNYGVHWVRQPPG |
| 1283 | | KALEWLGVIWSDGSTDYDTAFISRLTISKDNSKSQVVLTMTN |
| | | MDPVDTATYYCARRAVVADFDYWGQGTTVTVSS |
| SEQ ID NO: | VH-2 | QVQLQESGPGLVKPSGTLSLTCAVSGFSLTNYGVHWVRQPPG |
| 1284 | | KGLEWLGVIWSDGSTDYDTAFISRLTISKDNSKSQVSLKLSSV |
| | | TAADTAVYYCARRAVVADFDYWGQGTTVTVSS |
| SEQ ID NO: | VH-3 | QVQLQQSGPGLVKPSQTLSLTCAVSGFSLTNYGVHWVRQSPS |
| 1285 | | RGLEWLGVIWSDGSTDYDTAFISRLTINKDNSKSQVSLQLNSV |
| | | TPEDTAVYYCARRAVVADFDYWGQGTTVTVSS |
| SEQ ID NO: | VH-4 | EVQLVESGGGLVQPGPSLRLSCTVSGFSLTNYGVHWVRQAPG |
| 1286 | | KQLEWLGVIWSDGSTDYDTAFISRLTISKDNSKSIVYLQMNSL |
| | | KTEDTAVYYCARRAVVADFDYWGQGTTVTVSS |
| SEQ ID NO: | VH-5 | EVQLVQSGAEVKKPGESLRISCKVSGFSLTNYGVHWVRQMP |
| 1287 | | GKGLEWLGVIWSDGSTDYDTAFISQLTISKDNSISTVYLQWSS |
| | | LKASDTAMYYCARRAVVADFDYWGQGTTVTVSS |

MR5-2 (murine), Binds to human TCRVβ 13-2

| | | |
|---|---|---|
| SEQ ID NO: | SCFV (VH + VL) | QVQLQQSGTELMKPGASVKISCKASGYTFSNYWIEWIKQRPG |
| 1376 | | HGLEWVGEILPGAGPTNYNEKFKGKATFTADSSSNTAYMQLS |
| | | SLTSEDSAVYYCARTDYDYDWFAYWGQGTLVTVSAGGGGS |
| | | GGGGSGGGGSGGGGSDIVMSQSPSSLAVSVGEKVTMSCKSSQ |
| | | SLLYSGNQKNYLAWYQQKPGQSPKLLIYWASTRESGVPDRFT |
| | | GSGSGTDFTLTINSVKAEDLTVYYCQQYYGYPRTFGGGTKVE |
| | | IK |

TABLE 14
Exemplary Fc modifications

| Modification or mutation | Altered effector function |
|---|---|
| Leu235Glu | ADCC; |
| Leu234Ala/Leu235Ala (LALA) | ADCC; ADCP; CDC |
| Ser228Pro/Leu235Glu | |
| Leu234Ala/Leu235Ala/Pro329Gly | ADCP |
| Pro331Ser/Leu234Glu/Leu235Phe | CDC |
| Asp265Ala | ADCC, ADCP |
| Gly237Ala | ADCP |
| Glu318Ala | ADCP |
| Glu233Pro | |
| Gly236Arg/Leu328Arg | ADCC |
| His268Gln/Val309Leu/Ala330Ser/Pro331Ser | ADCC; ADCP; CDC |
| Val234Ala/Gly237Ala/Pro238Ser/ | ADCC; ADCP; CDC |
| His268Ala/Val309Leu/Ala330Ser/Pro331Ser | |
| Leu234Ala/L235Ala/Gly237Ala/P238Ser/ | ADCC; CDC |
| His268Ala/Ala330Ser/Pro331Ser | |
| Ala330Leu | CDC |
| Asp270Ala | CDC |
| Lys322Ala | CDC |
| Pro329Ala | CDC |
| Pro331Ala | CDC |
| Val264Ala | CDC |
| High mannose glycosylation | CDC |
| Phe241Ala | CDC |
| Asn297Ala or Gly or Gln | ADCC; ADCP; CDC |
| S228P/Phe234Ala/Leu235Ala | ADCC; CDC |

TABLE 15
Amino acid sequences of exemplary
variable regions of anti-BCMA antibodies.

| SEQ | | |
|---|---|---|
| ID NO | Description | Sequence |
| 3439 | 83A10 VH | EVQLLESGGGLVQPGGSLRLSCAASGFTFSSY |
| | | AMSWVRQAPGKGLEWVSAISGSGGSTYYADSV |
| | | KGRFTISRDNSKNTLYLQMNSLRAEDTAVYYC |
| | | AKVLGWFDYWGQGTLVTVSS |
| 3440 | 83A10 VL | EIVLTQSPGTLSLSPGERATLSCRASQSVSSS |
| | | YLAWYQQKPGQAPRLLIYGASSRATGIPDRFS |
| | | GSGSGTDFTLTISRLEPEDFAVYYCQQYGYPP |
| | | DFTFGQGTKVEIK |
| 3441 | 17A5 VH | EVQLLESGGGLVQPGGSLRLSCAASGFTFSSY |
| | | AMSWVRQAPGKGLEWVSAISGSGGSTYYADSV |
| | | KGRFTISRDNSKNTLYLQMNSLRAEDTAVYYC |
| | | AKVAPYFAPFDYWGQGTLVTVSS |
| 3442 | 17A5 VL | EIVLTQSPGTLSLSPGERATLSCRASQSVSSS |
| | | YLAWYQQKPGQAPRLLIYGASSRATGIPDRFS |
| | | GSGSGTDFTLTISRLEPEDFAVYYCQQYGNPP |
| | | LYTFGQGTKVEIK |
| 3443 | 13A4 VH | EVQLVQSGAEVKKPGESLKISCKGSGYSFTSY |
| | | WIGWVRQMPGKGLEWMGIIYPGDSDTRYSPSF |
| | | QGQVTISADKSISTAYLQWSSLKASDTAMYYC |
| | | ARNGYLGDYWGQGTLVTVSS |
| 3444 | 13A4 VL | DIVMTQSPLSLPVTPGEPASISCRSSQSLLHS |
| | | NGYNYLDWYLQKPGQSPQLLIYLGSNRASGVP |
| | | DRESGSGSGTDFTLKISRVEAEDVGVYYCMQA |
| | | MQIPTFGQGTKVEIK |
| 3445 | J22.9-xi | QVQLQQSGGGLVQPGGSLKLSCAASGIDFSRY |
| | VH | WMSWVRRAPGKGLEWIGEINPDSSTINYAPSL |
| | | KDKFIISRDNAKNTLYLQMSKVRSEDTALYYC |
| | | ASLYYDYGDAMDYWGQGTSVTVSS |
| 3446 | J22.9-xi | DIVMTQSQRFMTTSVGDRVSVTCKASQSVDSN |
| | VL | VAWYQQKPRQSPKALIFSASLRFSGVPARFTG |
| | | SGSGTDFTLTISNLQSEDLAEYFCQQYNNYPL |
| | | TFGAGTKLELKR |
| 3447 | 2A1 VH | EVQLVESGGGLVKPGGSLRLSCAASGFTFGDY |
| | | ALSWFRQAPGKGLEWVGVSRSKAYGGTTDYAA |
| | | SVKGRFTISRDDSKSTAYLQMNSLKTEDTAVY |
| | | YCASSGYSSGWTPFDYWGQGTLVTVSS |
| 3448 | 2A1 VL | QSVLTQPPSASGTPGQRVTISCSGSSSNIGSN |
| | | TVNWYQQLPGTAPKLLIFNYHQRPSGVPDRFS |
| | | GSKSGSSASLAISGLQSEDEADYYCAAWDDSL |
| | | NGWVFGGGTKLTVLG |

TABLE 16
Amino acid sequences of
exemplary anti-CD3ε molecule BJM1210

| SEQ | | |
|---|---|---|
| ID NO | Description | Sequence |
| 3400 | VH (CDR | QVQLQQPGTELVKPGASVKLSCKASGYTFT |
| | underlined) | TYWMHWVKQRPGHGLEWIGNFNPNNGDINY |
| | | NEKFKTKATLTVDKSSSTAYMQLSSLTSED |
| | | SAVYYCARDDYGRYYFDYWGQGTTLTVSS |
| 3401 | VL (CDR | DIVMSQSPSSLAVSAGEKVTMSCKSSQSLL |
| | underlined | NSRTRKNYLAWYQQKPGQAPKLLIYWAFTR |
| | | ESGVPDRFTGSGSGTDFTLTISSVQAEDLA |
| | | IYYCKQSFILRTFGGGTKLEIK |

### EXAMPLES

The present disclosure will be more specifically illustrated by the following Examples. However, it should be understood that the present disclosure is not limited by these examples in any manner.

### Example #1: Determination of Location TCRvβ-Specific Antibody Binding to TCRβ Through Epitope Mapping

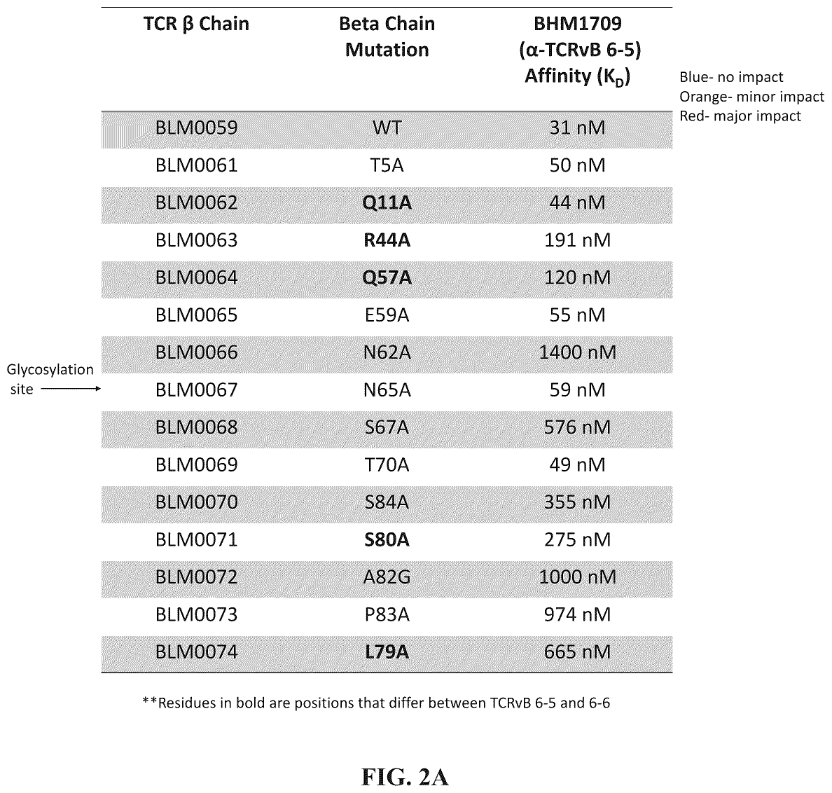

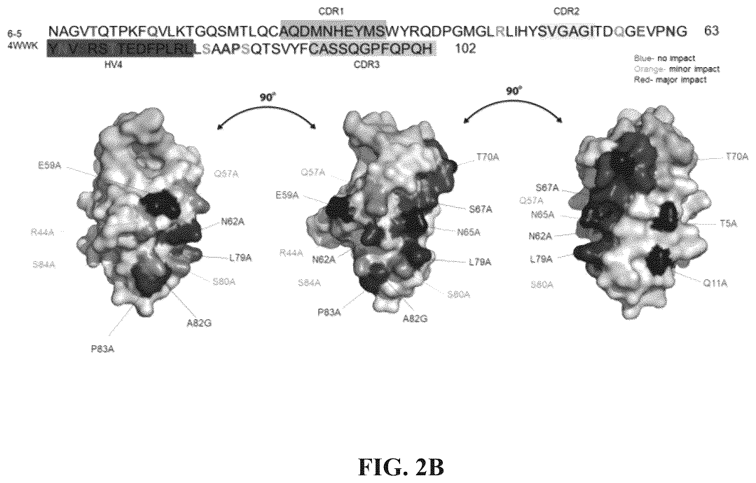

To narrow down where on the TCR β chain the TCRvβ-specific antibodies were binding, an alanine walk experiment was performed. Briefly, TCR β chains were mutated at various amino acids throughout the variable region. More specifically, amino acids were changed to alanine at various locations (except for the BLM0072 TCR, which mutated an alanine to a glycine) (FIG. 2A). The TCR alanine scanning variants were expressed recombinantly and then tested as purified proteins for binding to TCRvB6-5 and TCRvB6-6 by surface plasmon resonance to quantify binding as a K_{D}. The affinity between the mutated TCRs and the TCRvβ antibody, α-TCRvB6-5, were then determined. If the K_{D} value was colored blue, then the mutation at that location had no impact on a-TCRvB6-5 binding. If the K_{D} value was colored orange, then the mutation at that location had a minor impact on α-TCRvB6-5 binding. If the K_{D} value was colored red, then the mutation at that location had a major impact on α-TCRvB6-5 binding. Overall, the N62A, S67A, A82G, P83A, and the L79A mutations had the greatest impact on α-TCRvB6-5 binding to the TCR β chain. In order to better visualize the results from the alanine walk, a 3D structural model was generated that showed the location of the amino acid mutations on the actual TCR β chain structure (FIG. 2B). As seen on the model, most of the vital mutations were located on the bottom half of the β chain. Additionally, several of the vital amino acid mutations (S67A and L79A) were located near the HV4 region.

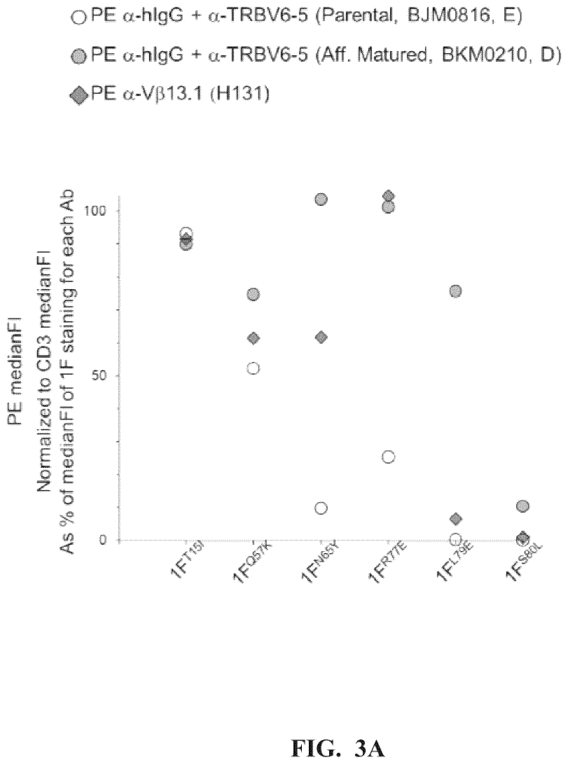

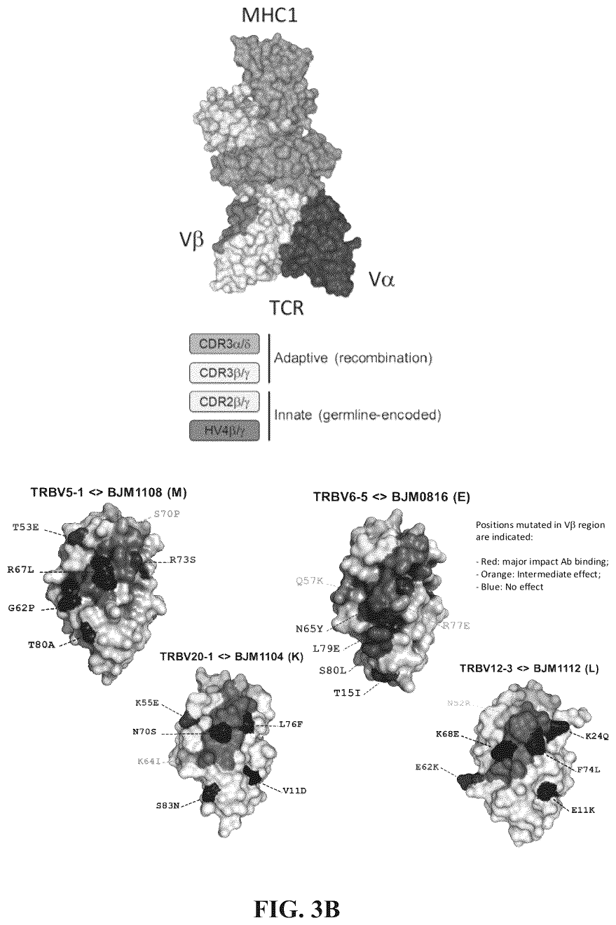

To further elucidate the binding location of α-TCRvβ6-5 on the TCR, six different lentiviral vectors were generated that each contained a different amino acid change (T15I, Q57K, N65Y, R77E, L79E, S80L); the mutations were chosen in order to compare closely related TCR clonotypes. J76 Jurkat cells (TCRα^{-/-}, TCRβ^{-/-}) were transduced with the lentiviral vectors expressing TCRα and variant TCRβ chains. Upon successful transduction, the six variant J76 cell lines were incubated with the anti-TCRvβ antibodies (primary) followed by PE-α-hIgG1 for FACS detection. The binding of three TCRv3 antibodies were tested in this experiment: PE a-hIgG+α-TCRvβ6-5 (parental version of antibody, BJM0816). PE α-hIgG+α-TCRvβ6-5 (affinity matured version of antibody, BKM0210), and PE α-Vβ13.1. These three antibodies were added to the transduced J76 cells and incubated overnight. The cells were rinsed with PBS after adding the secondary detection antibody (a-PE) and incubated protected from light. After incubation, the cells were washed and then mounted in fluorescent antifade mounting media and then imaged to determine median fluorescence intensity (medianFI). The PE medianFI was normalized using the medianFI of α-CD3 binding, meaning he ability of the TCRvβ antibodies to bind to these different variant TCRs was normalized to the ability of α-CD3 antibody to bind to the same variant TCRs. This experiment and analysis was repeated for all TCRvβ variant antibodies disclosed. In FIG. 3A, the medianFI of α-TCRvβ6-5 (BJM0816) (white dots), α-TCRvβ6-5 (BKM0210) (gray dots), and α-Vβ13.1 (blue diamonds) were plotted. All three antibodies bound to the T15I variant J76 cell variant, bound the Q57K J76 variant at above 50%, and were unable to bind efficiently to the S80L J76 variant. α-TCRvβ6-5 (BKM0210) was able to bind to the N65Y and R77E variants at close to 100% and the L79E variant at around 75%. α-TCRvβ6-5 (BJM0816) was unable to bind efficiently to the N65Y, R77E, or the L79 variants. α-Vβ13.1 was able to bind to the N65Y variant at around 60/o and the R77E variant at close to 100%: however it was unable to bind efficiently to the L79E variant.

The cumulative results from the epitope mapping experiments were developed into 3D structural models for the TCRs TCRvβ5-1 (BJM1108), TCRvβ6-5 (BJM0816), TCRvβ20-1 (BJM1104), and TCRvβ312-3 (BJM1112) (FIG. 3B). The single amino acid mutations were shown with dotted lines and the positions mutated in the Vβ region were indicated by the color of their text. If a mutation was labeled blue, such as the T15I mutation in TCRvβ6-5 structural model, then that amino acid mutation had no effect on the TCRvβ antibody binding at that location; if a mutation was labeled orange, such as the K641 mutation in the TCRvβ20-1 structural model, then that amino acid mutation had an intermediate effect on the TCRvβ antibody binding at that location: if a mutation was labeled red, such as the R73S mutation in the TCRvβ5-1 structural model, then that amino acid mutation had a major impact on TCRvβ antibody binding at that location. For the TCRvβ5-1 TCR, the R67L, G67P, and T80A mutations had no effect on TCRvβ-specific antibody binding, the S70P mutation had an intermediate effect on binding, and the T53E and R73S mutations had major impact on binding. For the TCRvβ6-5 TCR, the T15I mutation had no effect on binding, the Q57K and R77E mutations had an intermediate effect on binding, and the N65Y. L79E, and S80L mutations had major impacts on binding. For the TCRvβ20-1 TCR, the L76F, N70S, and S83N mutations had no effect on binding, the K641 mutation had an intermediate effect on binding, and the K55E mutation had a major impact on binding. For the TCRvβ12-3 TCR, the K24Q, F74L, and E11K mutations had no effect on binding, the N52R mutation had an intermediate effect on binding, and the E62K mutation had a major effect on binding.

### Example #2: TCRvb Drives Expansion and Proliferation of vB-Specific Cells

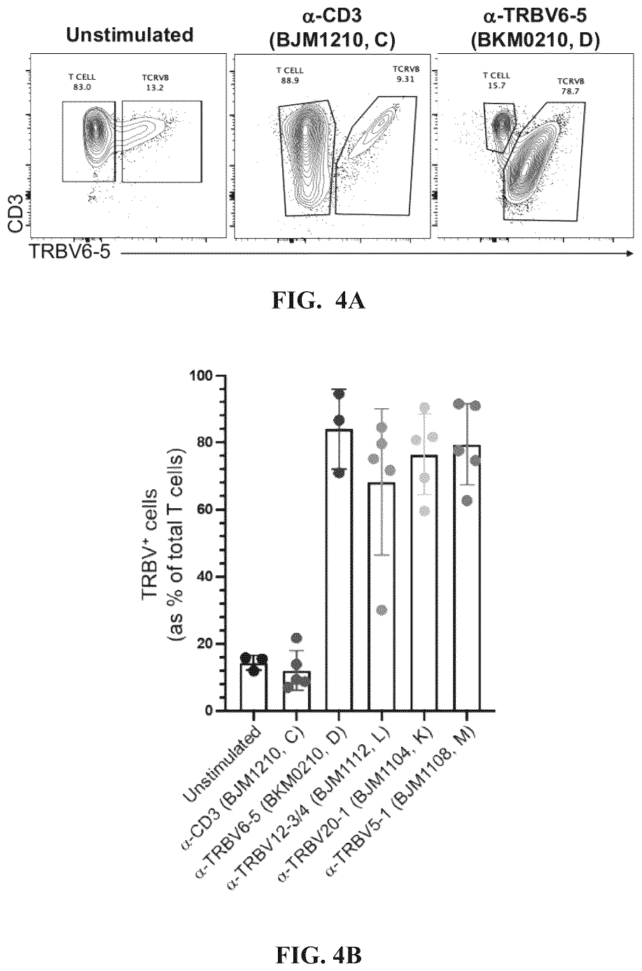

To test the effect of TCRvβ antibodies on T cell expansion. T cell were cultured for 7 days in one of the following conditions: no antibody, cultured with α-CD3 antibodies, or cultured with a α-TCRvβ antibody. After 7 days, the cells were stimulated with PMA/IONO/BFA and the cells were harvested and incubated with conjugated primary antibodies, α-CD3 and α-TCRvβ. After washing off any unbound antibody, the cells were centrifuged and then resuspended in Flow Cytometry Staining Buffer for the final flow cytometric analysis. FIG. 4A shows an example of the results obtained by flow cytometric analysis. The cells were gated on either CD3 only expression or TCRVB expression. The T cells that were stimulated with α-CD3 antibodies had over 88% of cells expressing only CD3. On the contrary, the T cells that were stimulated with α-TCRv6-5 had only about 15% of T cells that only express CD3; over 78% of TCRvβ6-5⁺ T cells. This showed that the addition of α-TCRvβ antibodies in culture increased the expansion of TCRvβ⁺ T cells compared to cultures with just α-CD3 antibody. T cell expansion assays were performed for each TCRvβ antibody variants and compared to unstimulated cells and cells stimulated with α-CD3 (FIG. 4B). All four TCRvβ antibodies tested had a higher number of TCRvβ⁺ positive cells compared to cells that were unstimulated or stimulated with α-CD3.

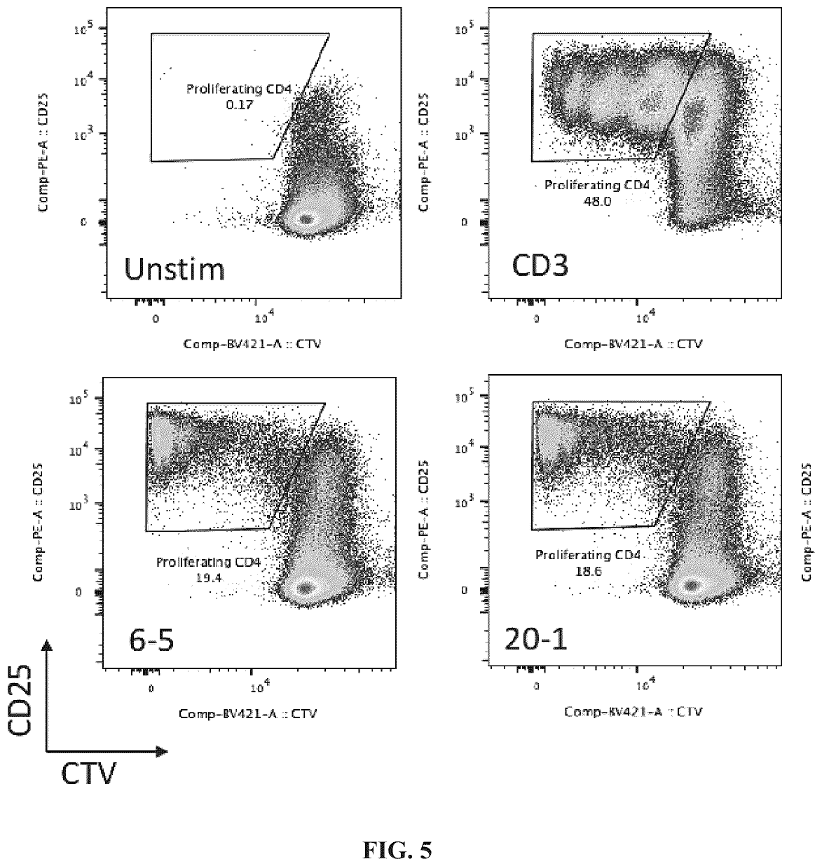

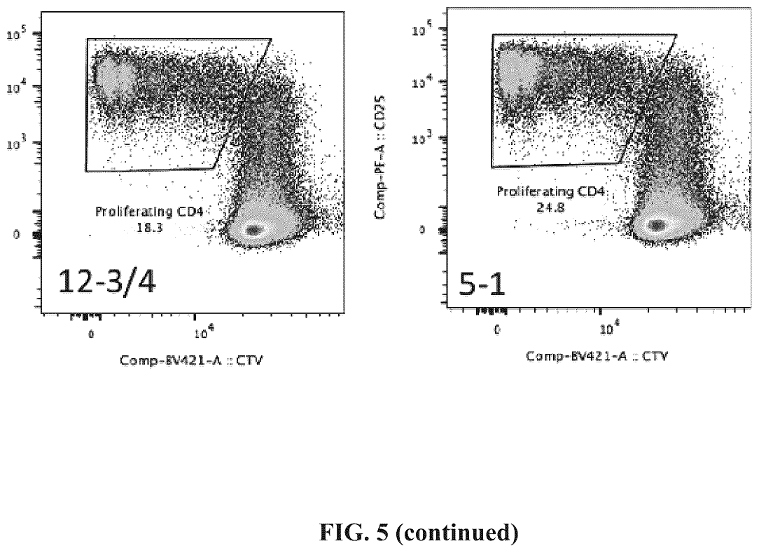

TCRvβ antibodies were also tested for their ability to drive T cell proliferation. T cells were incubated with CellTrace Violet (CTV) before being stimulated for 5 days with α-CD3 antibody or a α-TCRvβ antibody. After the stimulation, the cells were harvested and stained with α-CD25 and α-CTV antibodies. The α-CTV antibody was used to measure cell proliferation as dividing cells lose CTV at every division. The cells were gated on CD25⁺ and CTV⁻, which showed proliferating CD4⁺ T cells (FIG. 5). The unstimulated cells showed no proliferation, while cells cultured with α-CD3 had 48% of all T cells as proliferating CD4⁺ T cells. The cells that were incubated with the α-TCRvβ antibodies had lower percentage of proliferating CD4⁺ T cells compared to the α-CD3 control, which showed that the cells cultured with α-TCRvβ antibodies had proliferated at a higher rate.

### Example #3: TCRvB Stimulation Results in Hyper Activated State

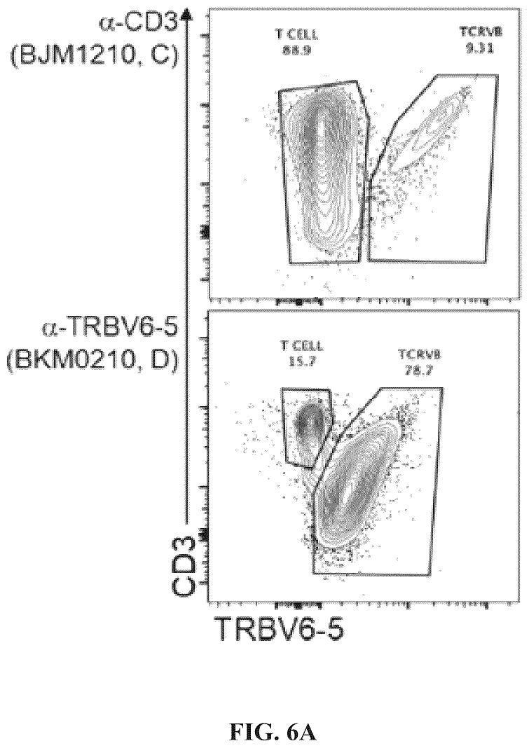

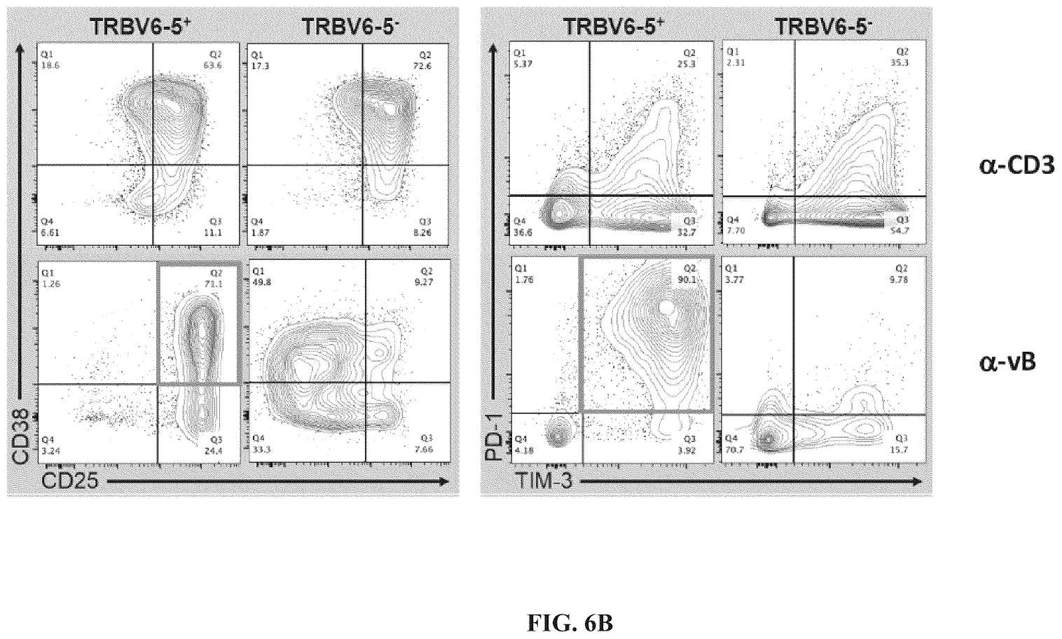

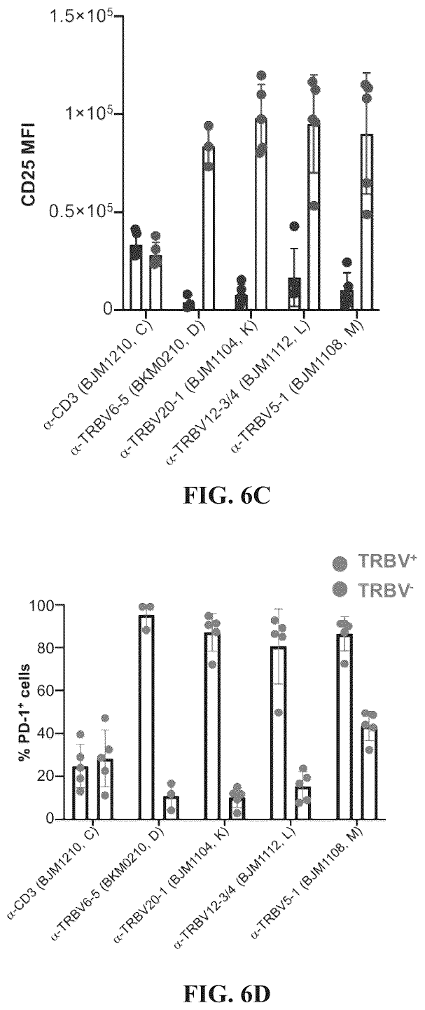

To determine the phenotype of the T cells cultured with TCRvβ antibodies, T cells were either cultured with α-CD3 or an α-TCRvβ antibody bound to the culture plate. After 7 days, the cells were stimulated with PMA/IONO/BFA and stained with α-CD3 and α-TCRvβ antibodies. These stained cells were FACS sorted into TCRvβ⁺ or TCRvβ⁻ cells from the two different conditions (FIG. 6A). The sorted cells were stained with α-CD38, α-CD25, a-PD-1, and a-TIM-3 antibodies to determine the activation state of the T cells. This experiment was repeated for all the TCRvβ variant antibodies. An example of the results for one of the TCRvβ antibodies, TCRvβ6-5, was shown in FIG. 6B, TCRvβ⁻ T cells that developed from stimulation from TCRvβ6-5 culture had higher levels of surface level expression of CD38, CD25, PD-1, and TIM-3 compared to TCRvβ⁻ T cells that developed from the α-TCRvβ culture, TCRvβ⁻ T cells that developed from α-CD3 culture, or TCRvβ⁺ T cells that developed from α-CD3 culture. In addition, TCRvβ⁺ cell cultured with a α-TCRvβ antibody had higher median fluorescence intensity of CD25 (FIG. 6C) and a higher percentage of PD-1⁺ cells (FIG. 6D) compared to TCRvβ⁻ T cells that developed from the α-TCRvβ6-5 culture, TCRvβ⁺ T cells that developed from the α-CD3 culture, or TCRvβ⁺ T cells that developed from the α-CD3 culture. This finding showed that TCRvβ was able to induce a hyper activated phenotype compared to α-CD3. In addition, these experiments highlighted CD25 as an activation marker for TCRvβ T cells.

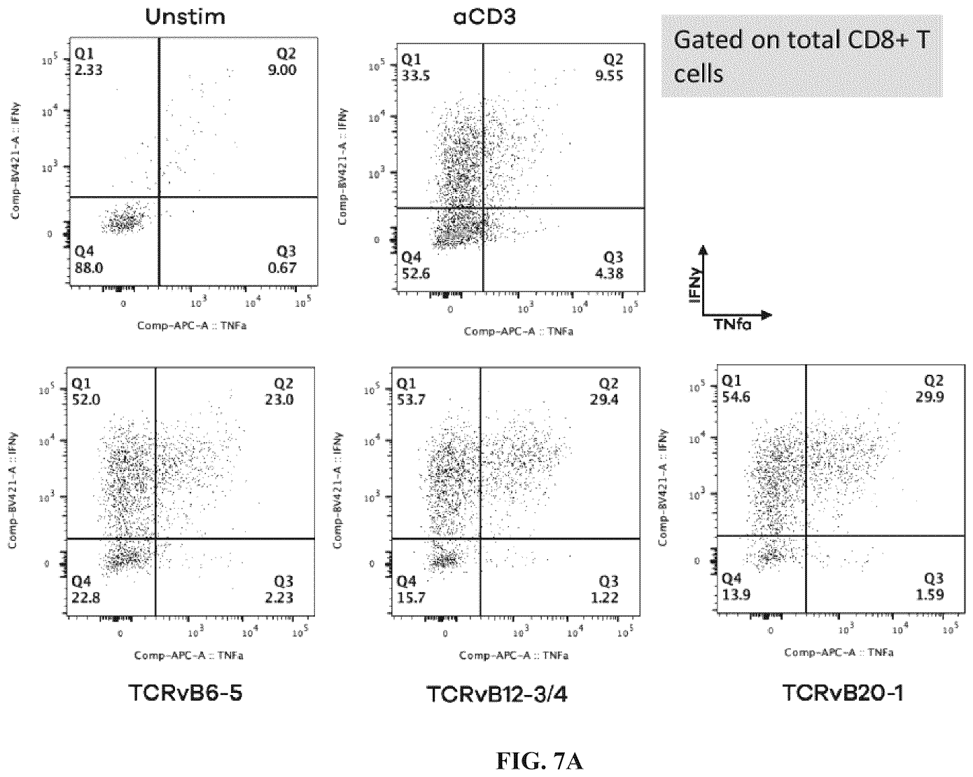

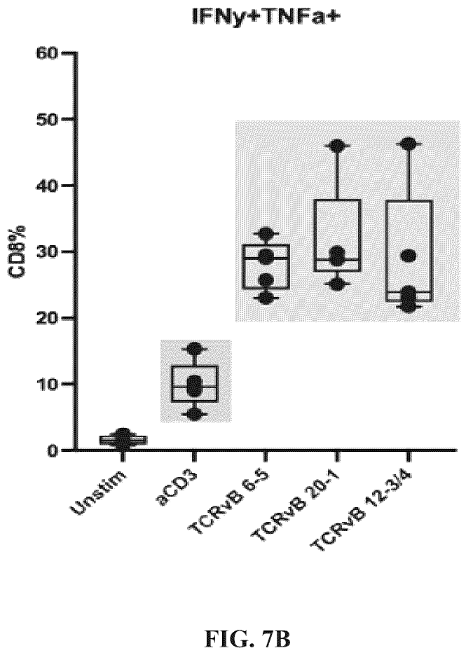

The functional effect on T cells of TCRvβ was determined by growing T cells for 7 days in one of the following conditions: with no antibodies, with α-CD3 antibodies, or with a α-TCRvβ antibody. For growth stage, the antibodies were bound to the plate. The cells were then either not stimulated or stimulated with PMA/IONO/BFA. After staining for cell surface markers, cells were resuspended in Intracellular Staining Perm Wash Buffer and centrifuged. The cells were then stained with α-IFNγ, α-TNFα, α-IL-2, α-IL-17, and α-IL-4. Before analysis, the cells are washed with Intracellular Staining Perm Wash Buffer, centrifuged, and resuspended. To determine the functionality of cytolytic T cells, the cells were gated on CD8⁺ T cells. Three TCRvβ antibodies were tested in this experiment: TCRvβ6-5, TCRvβ20-1, and TCRvβ12-3/4. For the unstimulated and α-CD3 cultured cells, the majority of the CD8⁺ T cells were IFNγ ⁻TNFα⁻ (FIG. 7A). However, the cells cultured with α-TCRvβ resulted in over 50% of CD8⁺ T cells being IFNγ⁺ TNFα⁻ and over 20% of CD8⁺ T cells being IFNγ⁺ TNFα⁺ (FIGS. 7A and 7B). No difference was observed in IL-2, IL-4, or IL-17 producing cell with α-TCRvβ or α-CD3 cultures. Overall, TCRvβ produced a higher number of CD8⁺ T cells that produced IFNγ and TNFα compared to α-CD3.

### Example 4: TCRvβ Drives a Central Memory-Like Phenotype

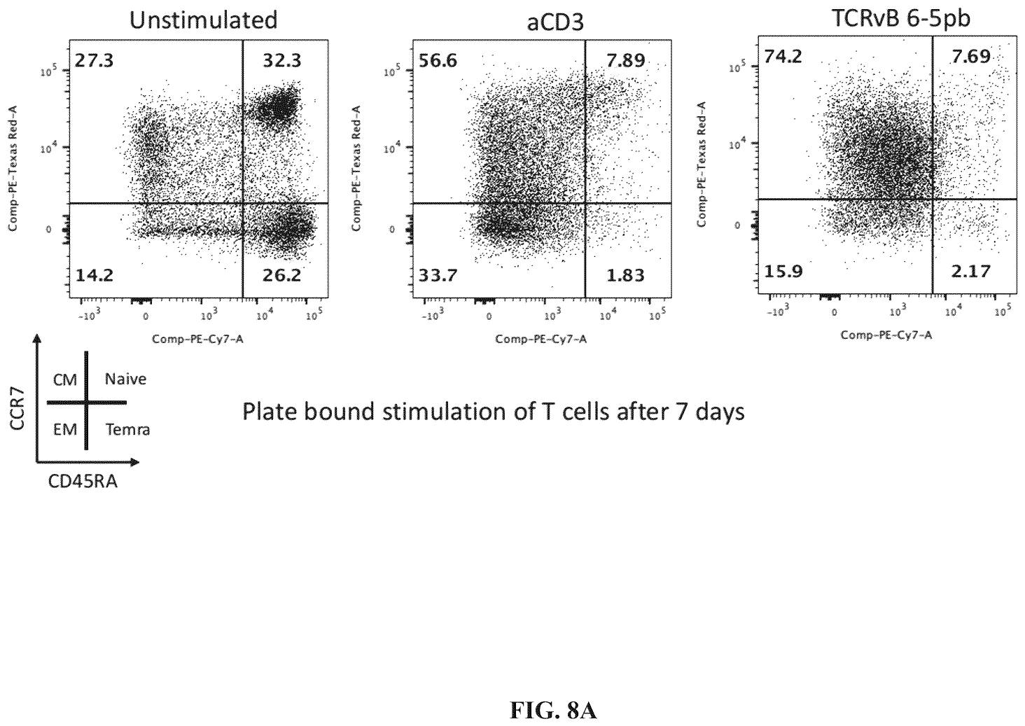

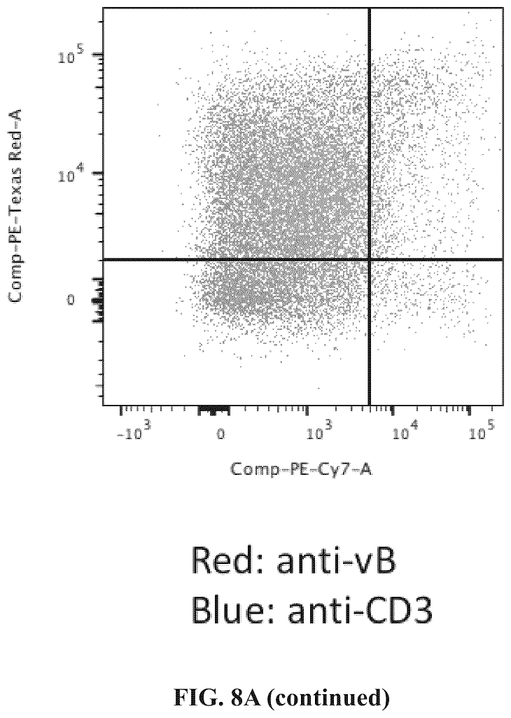

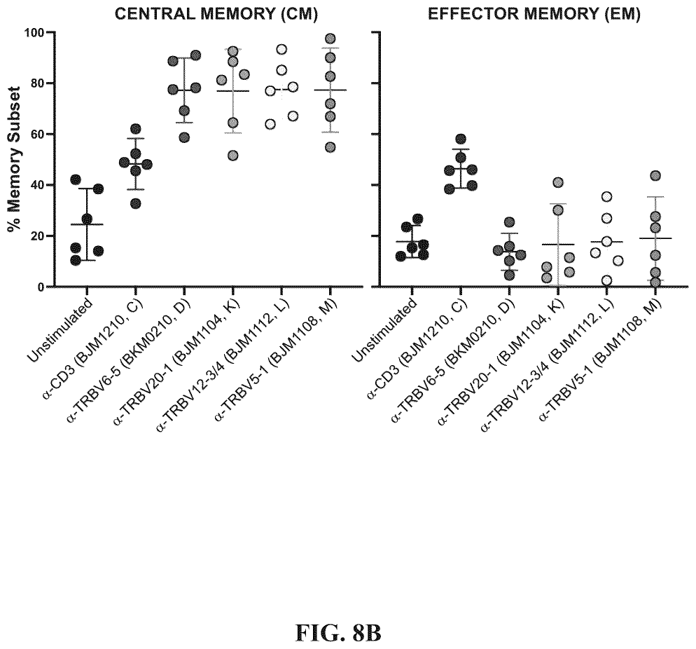

T cells were cultured for 7 days in one of the following conditions: with no antibody, with α-CD3, or with α-TCRvβ. For the growth period, the antibodies were bound to the plate. After 7 days, the cells were stimulated with PMA/IONO/BFA and then stained with α-CCR7 and α-CD45RA to determine which T memory cell phenotype the TCRvβ-cultured cells most closely resemble. For the unstimulated cells, the majority of cells were CCR7⁺ CD45RA⁺ or CCR7⁻ CD45RA⁺ meaning they had naïve T cells or terminally differentiated effector memory (TEMRA) T cells phenotypes, respectively. For the α-CD3 cells, over 56% of the cells were CCR7⁺ CD45RA⁻ or had a central memory T cell phenotype, and over 33% were CCR7⁻ CD45RA⁻ or had an effector memory T cell phenotype. For the α-TCRvβ⁺ cells, over 74% of the cell were CCR7⁺ CD45RA⁻ or had a central memory T cell phenotype (FIG. 8A). In addition, when the cells from the α-TCRvβ culture were further stained with a-VB (red) and α-CD3 antibodies (blue), the majority of the cells that were CCR7⁺ CD45RA⁻ were TCRvβ⁺ cells rather than TCRvβ⁻ cells (FIG. 8A, bottom graph). As shown in FIG. 8B, cultures with α-TCRvβ antibodies generated almost 80% cells with a central memory phenotype from all memory subset T cells (compared to only around 20% generation of T cells with an effector memory-like phenotype). Unstimulated cells generated only around 20% central memory T cells and α-CD3 cultures only generated around 50%. This result showed that TCRvβ drove the activated T cells to have a central memory T cell phenotype.

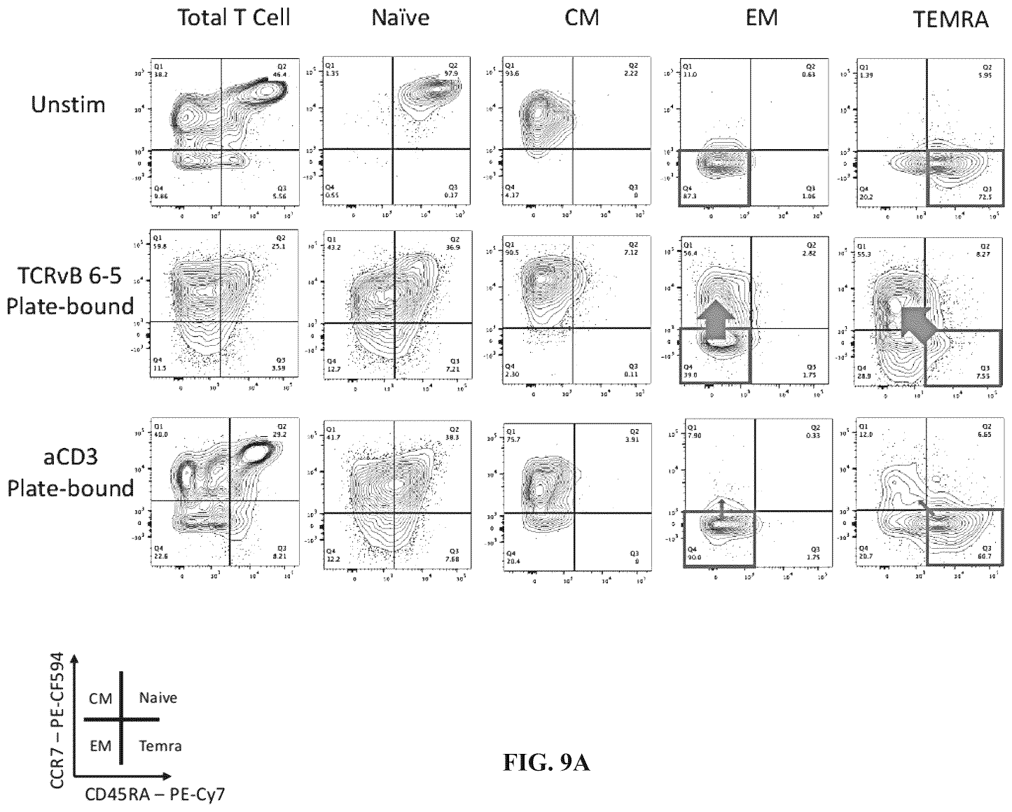

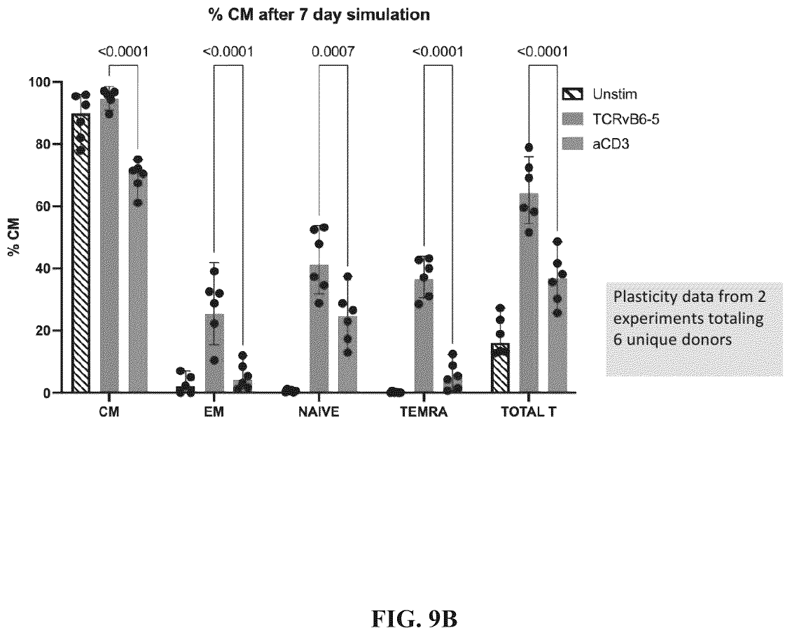

To further elucidate the phenotype of the TCRvβ stimulated cells, total, naïve, central memory (CM), effector memory (EM), and TEMRA T cells were FACS sorted and then cultured for 7 days. The sorted T cells were cultured with one of the following conditions: no antibodies, α-CD3, and α-TCRvB6-5. For the growth period, the antibodies were bound to the plate. After 7 days, the days were stimulated with PMA/IONO/BFA. For total T cell, naïve T cell, and central memory T cell populations, there were no major differences in T cell phenotype for cells stimulated with either α-CD3 or α-TCRvB6-5 antibodies. However, differences were seen when effector memory T cells and TEMRA T cells were stimulated. For effector memory T cells, when the T cells were unstimulated or cultured with α-CD3, the majority of the EM T were CCR7⁻ CD45RA⁻. Contrary to the unstimulated and α-CD3 cells, TCRvB6-5 culture resulted in most EM T cells becoming CCR7⁺ CD45RA⁺. While for the TEMRA T cells, the majority of unstimulated or α-CD3 T cells were CCR7⁺ CD45RA⁺. However, the majority of TCRvB6-5 T cells were CCR7⁺ CD45RA⁻, which was the phenotype for central memory T cells (FIG. 9A). In addition, α-TCRvB6-5 resulted in a significant increase in the percentage of central memory T cells induced from each subset of memory T cells (FIG. 9B). These findings showed that TCRvB6-5 induced effector memory T cells and TEMRA T cells to develop a central memory T cell-like phenotype, but that TCRvB6-5 also increased the percentage of central memory T cell induced from all T cell memory states or induced plasticity in the different T cell memory states.

### Example #5: Transcriptomic Analysis of TRCvβ T Cells

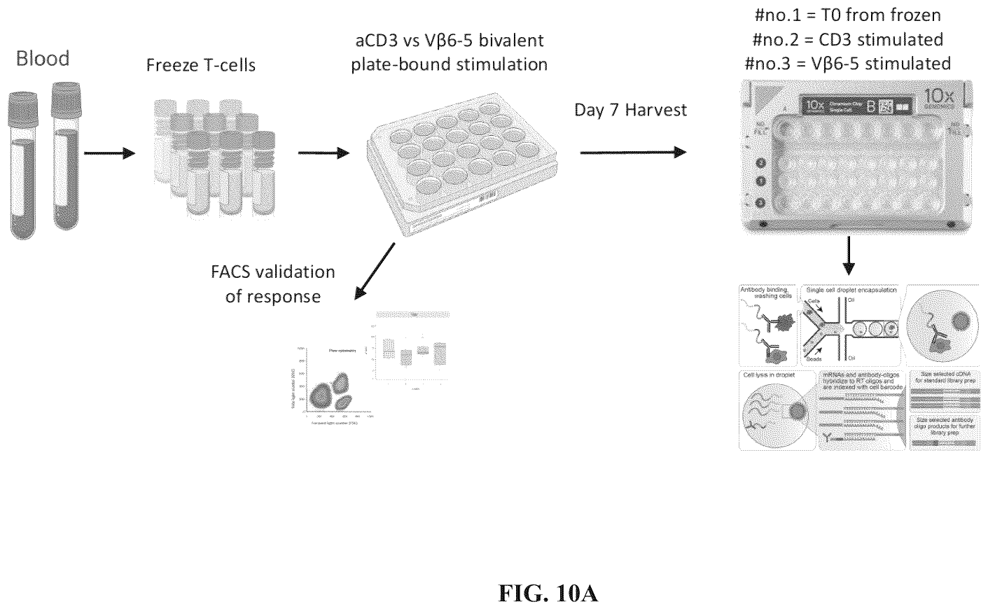

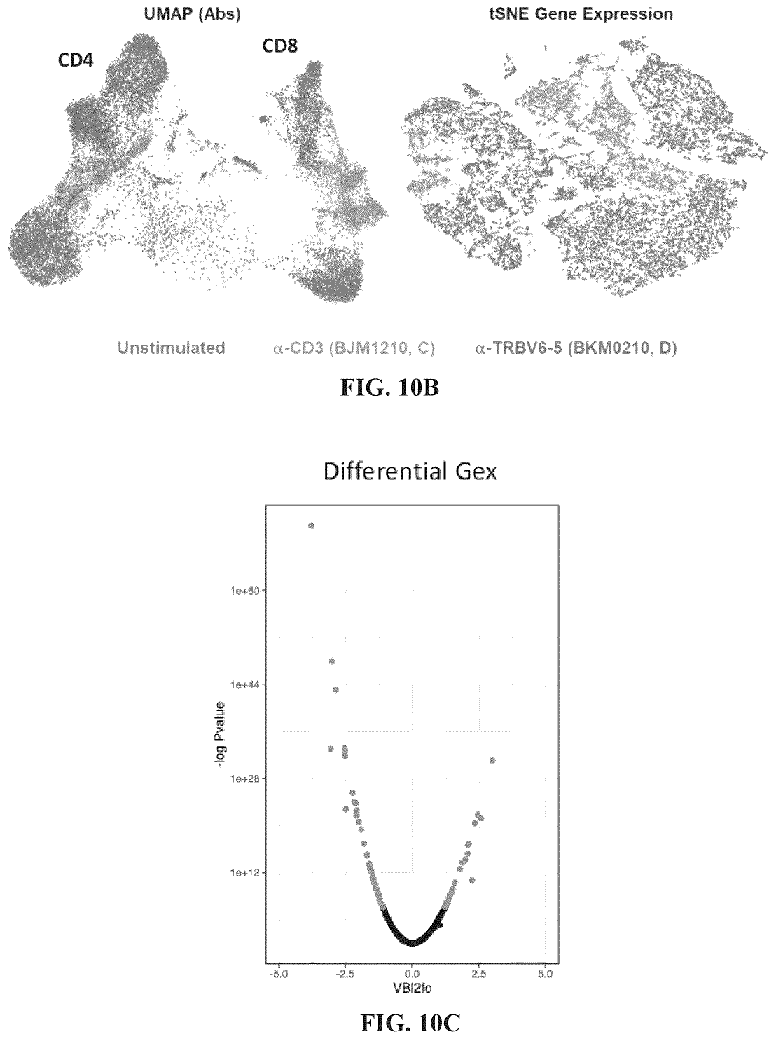

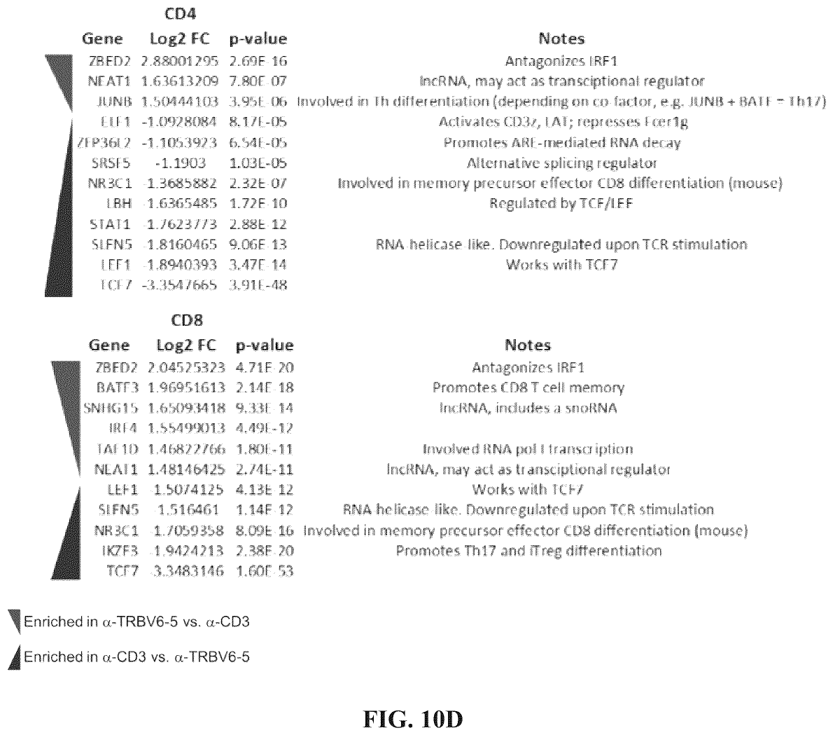

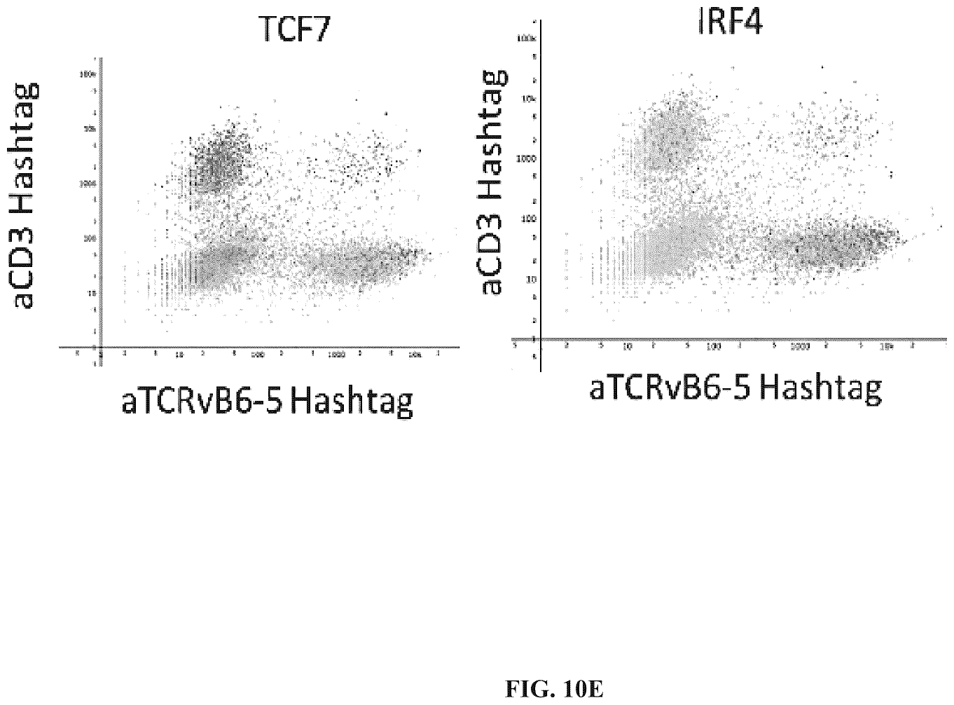

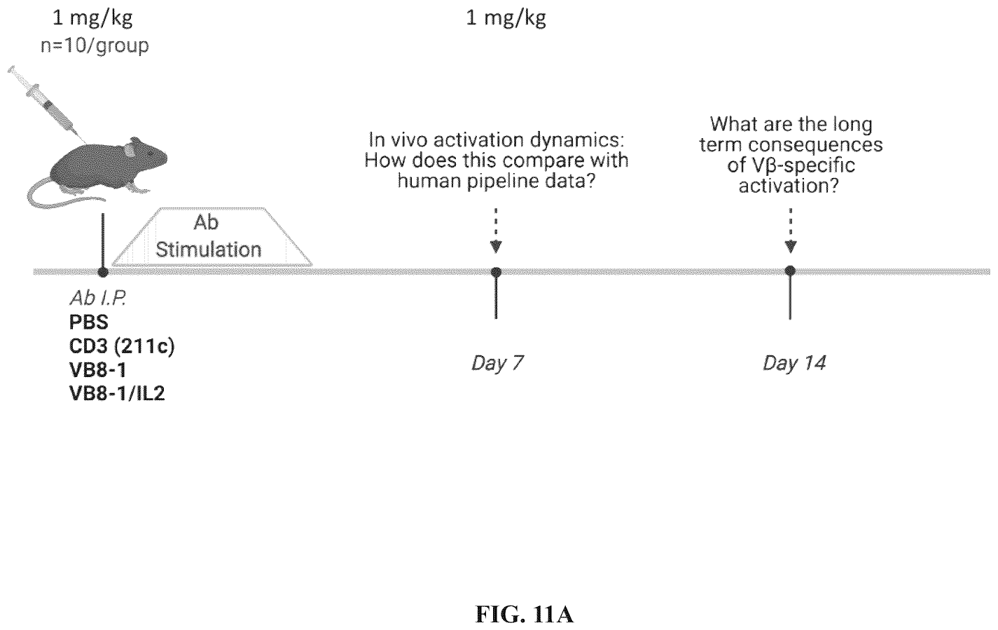

T cells were isolated from blood samples and then frozen. Frozen T cells were thawed and then either cultured with α-CD3 antibodies or α-Vβ bivalent plate-bound antibodies. The cells were cultured for 7 days, stimulated with PMA/IONO/BFA, and then used either for FACS validation of α-Vβ6-5 stimulation response (like the experiments presented below) or single-cell transcriptomics. Transcriptomic experiments were performed on α-CD3 T cells, α-V6-5β T cells, or freshly thawed unstimulated T cells. The single cells were then compartmentalized into single droplets which contained a single T cell, lysis buffer, and a microbead covered with barcoded primers. Following compartmentalization, the cells in the droplets were lysed and the released mRNA hybridized to the primers on the microbead. Following hybridization of mRNA to the microbead, all droplets were pooled and broken to release the beads. After the microbeads were isolated, the mRNA were reverse-transcribed; this generated the first cDNA strand with a PCR primer sequence, cDNAs underwent two rounds of size selection: first based on standard library preparation, followed by a second size-selection that used an antibody. Once the library preparation was completed, the samples were sequenced (FIG. 10A).

Initial transcriptomic multiplex analyses were performed and UMAP and tSNE models were generated, as well as a volcano plot (FIGS. 10B and 10C). The UMAP and tSNE models labeled unstimulated T cell genes shown in blue. α-CD3 T cell genes shown in orange, and α-TCRvβ6-5 T cells shown in green. Both the UMAP and tSNE plots showed that gene expression from all three different stimulation conditions clustered separately (FIG. 10B). The UMAP plot additionally showed that genes from the different culture conditions isolated from CD4′ and CD8⁺ T cells clustered separately from each other. Overall, both plots showed that cells cultured with α-vβ6-5 induced a distinct gene expression profile from α-CD3 or unstimulated T cells. The volcano plot showed differential gene expression in the α-vβ6-5 T cells compared to α-CD3 T cells (FIG. 10C). The red dots showed the most statistically significant downregulated (left) and upregulated (right) genes.

The upregulated and downregulated genes in α-vβ6-5 CD4⁺ and CD8⁺ T cells were then researched to determine their functions (FIG. 10D). Several of the listed genes were involved in immune response, TCR signaling, or RNA transcription. Scatterplots for two specific genes, TCF7 and IRF4, and their relative expression levels in α-CD3 T cells vs α-vβ6-5 T cells were separately plotted. As shown in both CD4⁺ and CD8⁺ T cells. TCF7 expression was higher in α-CD3 cells compared to α-TCRvβ6-5 cells (FIG. 10E, left graph). IRF4 expression was higher in α-TCRvβ6-5 CD8⁺ T cells compared to α-CD3 CD8⁺ T cells (FIG. 10E, right graph). These initial transcriptomics results showed that TCRvβ T cells had a distinct gene expression profile, with multiple genes involved in immune responses, TCR signaling, and RNA transcription.

### Example #6: Validation of In Vitro Human Findings Through In Vivo Mouse Experiments

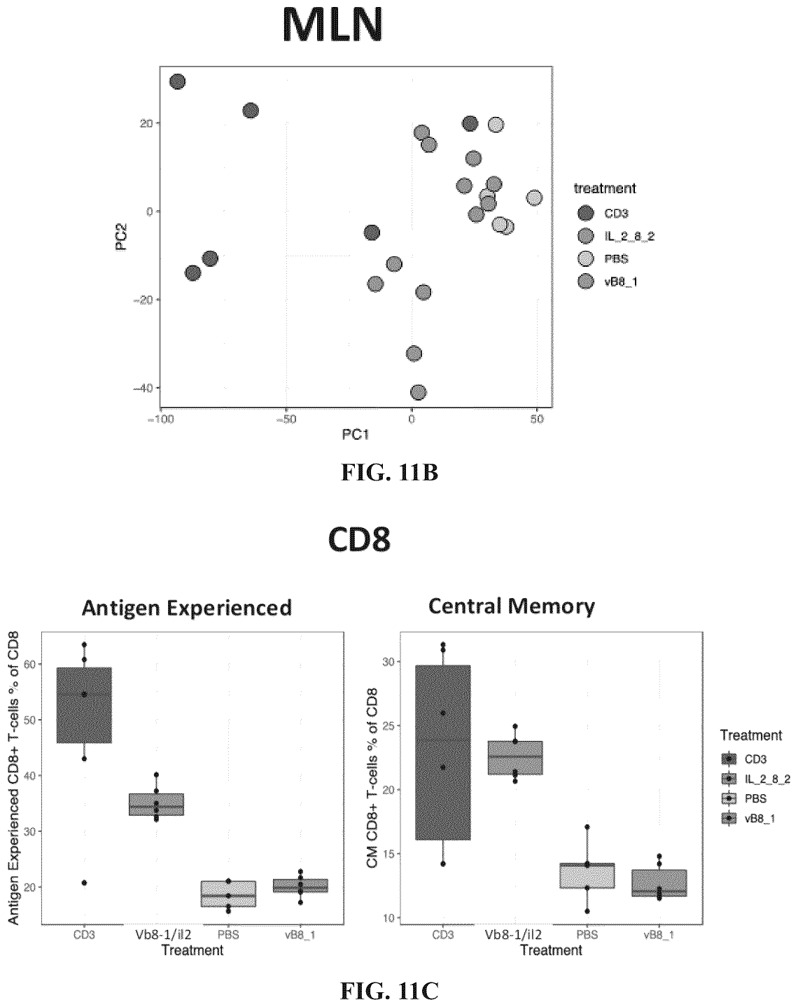

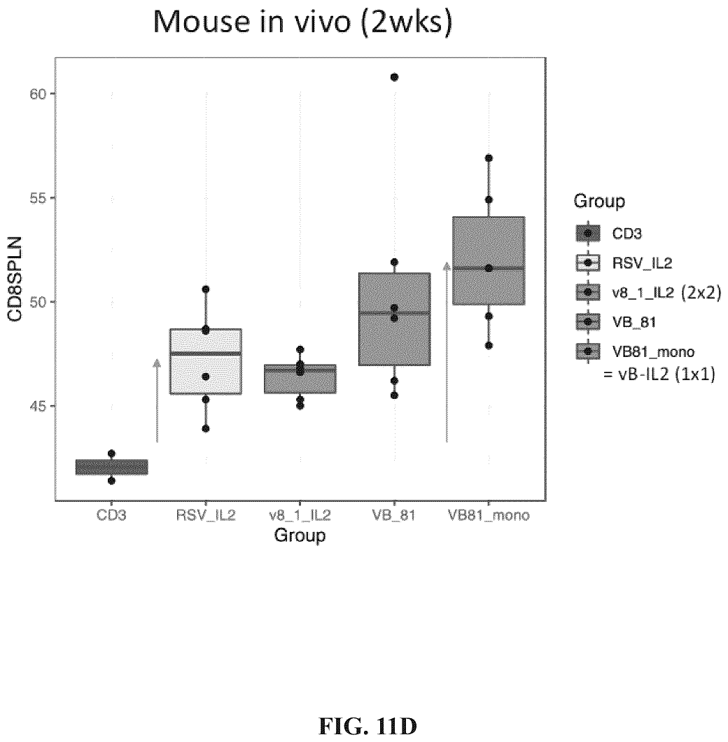

To determine the phenotype of TCRvβ stimulated T cells in vivo, groups of 10 mice were administered one of the following treatments intraperitoneally at 1 mg/kg: PBS, α-CD3 antibody, α-VB8-1 antibody, or α-VB8-1/IL-2 fusion molecule (FIG. 11A). 7 days after treatment, blood was collected (1 mg/kg) to evaluate the in vivo activation dynamics, and 14 days after treatment, biological samples were collected to determine the long-term consequences of Vβ-specific activation. PCA analysis of lymph nodes collected 14 days after injection were performed (FIG. 11B). Responses to PBS and α-vB8-1 treatments clustered together, while α-vB8-1/IL-2 treatment response clustered separately from the α-CD3 treatment and the other α-vB8-1 treatment. Using the 7-day post treatment blood samples, the percentage of antigen experienced CD8⁺ T cells and percentage of central memory CD8⁺ T cells were determined (FIG. 11C). For antigen experienced CD8⁺ T cells, treatment with α-CD3 generated the highest percentage of antigen experienced CD8⁺ T cells of all the treatments: neither the PBS or α-vB8-1 treatments generated many antigen experienced CD8⁺ T cells. The combination treatment of α-vB8-1/IL-2 generated a greater percentage of antigen experienced CD8⁺ T cells compared to PBS or α-vB8-1 alone treatments, although not as high as α-CD3 treatment (left graph of FIG. 11C).

Similar results were observed when the percentage of central memory CD8⁺ T cells out of all CD8⁺ T cells were calculated (right graph of FIG. 11C). α-CD3 treatment resulted in the highest percentage, α-vB8-1/IL-2 treatment resulted in the second highest percentage, and PBS and α-vB8-1 alone treatments resulted in lowest percentages. α-vB8-1/IL-2 treatment did not result in widespread activation of CD8⁺ T cells like α-CD3 treatment. These results highlighted the specificity and magnitude of response of α-vB8-1/IL-2 treatment in mice, especially since it was only targeting a relatively small subset of T cells.

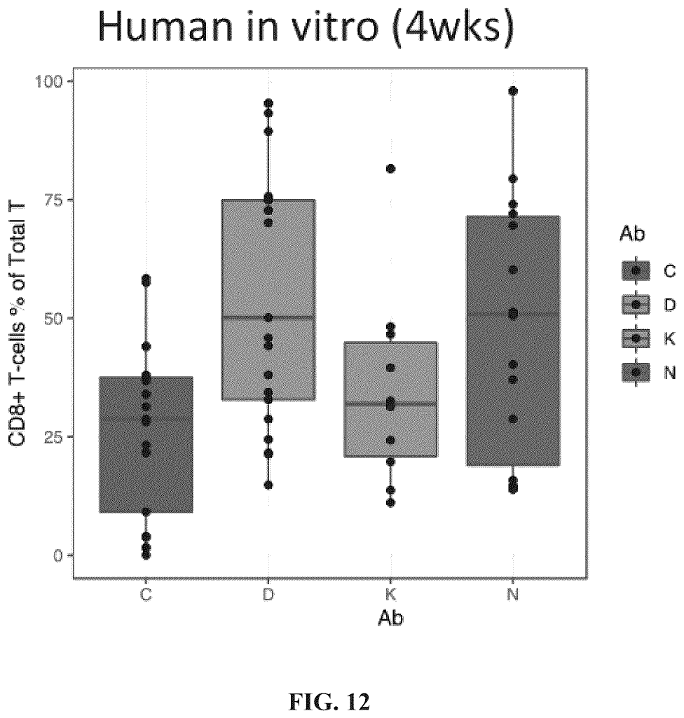

To validate the CD8⁺ T cell skewing observed in vitro, mice were administered one of the following treatments: α-CD3, α-RSV/IL-2, α-vB8-1, α-vB8-1/IL-2 fusion molecule (one Fc arm contained a vβ region and the other contained a fused IL-2 molecule, 1×1 bispecific), or α-vB8-1/IL-2 (both Fc arms contained a vβ region and a fused IL-2 molecule, 2×2 bispecific). The RSV/IL2 treatment group was dosed again at day 7 at 1 mg/kg. Splenic lymph nodes (SPLN) were collected from the mice 14 days after injection and the number of CD8⁺ T cells were calculated. Mice treated with α-CD3 had the lowest level of CD8⁺ T cells in the SPLNs and the mice treated with α-vB8-1 or α-vB8-1/IL-2 fusion molecule (1×1 bispecific) had the highest level of CD8 T cells in the SPLN (FIG. 11D). These results were compared to the percentage of CD8⁺ T cell out of total T cells determined by stimulating human T cells isolated from PBMCs for 4 weeks in vitro. The human T cells were cultured using one of the following: α-CD3, α-vB6-5, α-vB20-1, or α-vB6/hIL2 fusion (2×2 bispecific). The cells were then stimulated with PMA/IONO/BFA. T cells cultured with α-CD3 had the lowest percentage of CD8⁺ T cells, while T cells cultured with u-vB6-5 or α-vB6/hIL2 fusion had the highest percentages of CD8⁺ T cells (FIG. 12). Findings from both the in vivo and in vitro studies confirmed that stimulation with TCRvβ antibodies resulted in CD8⁺ T cell skewing.

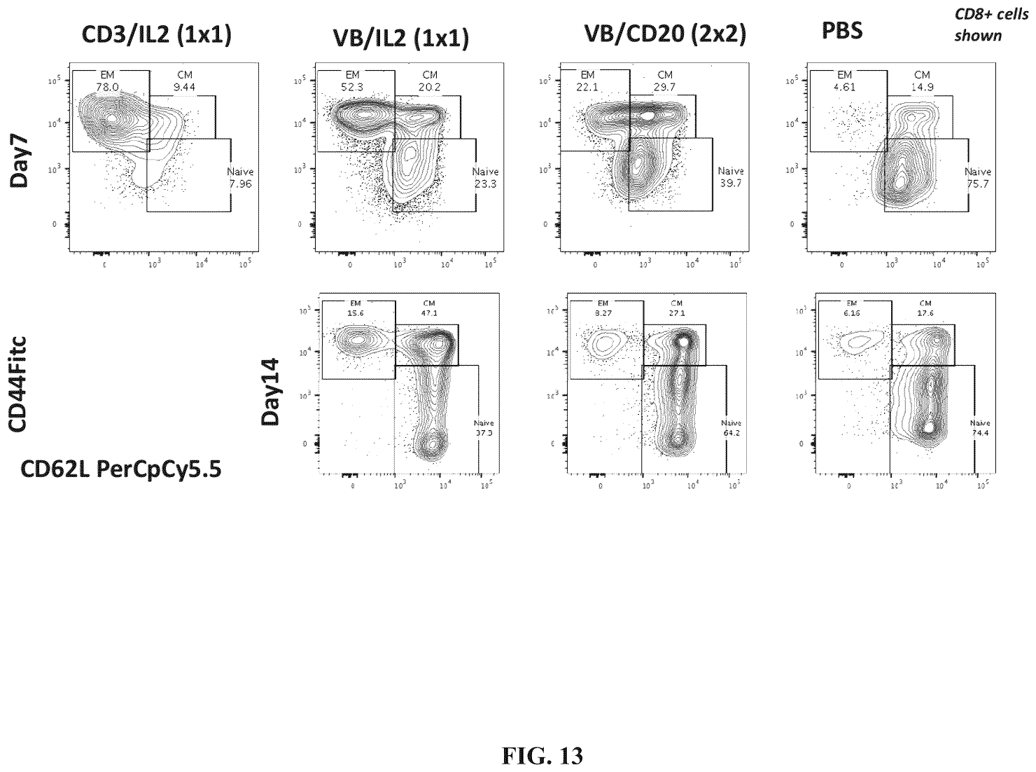

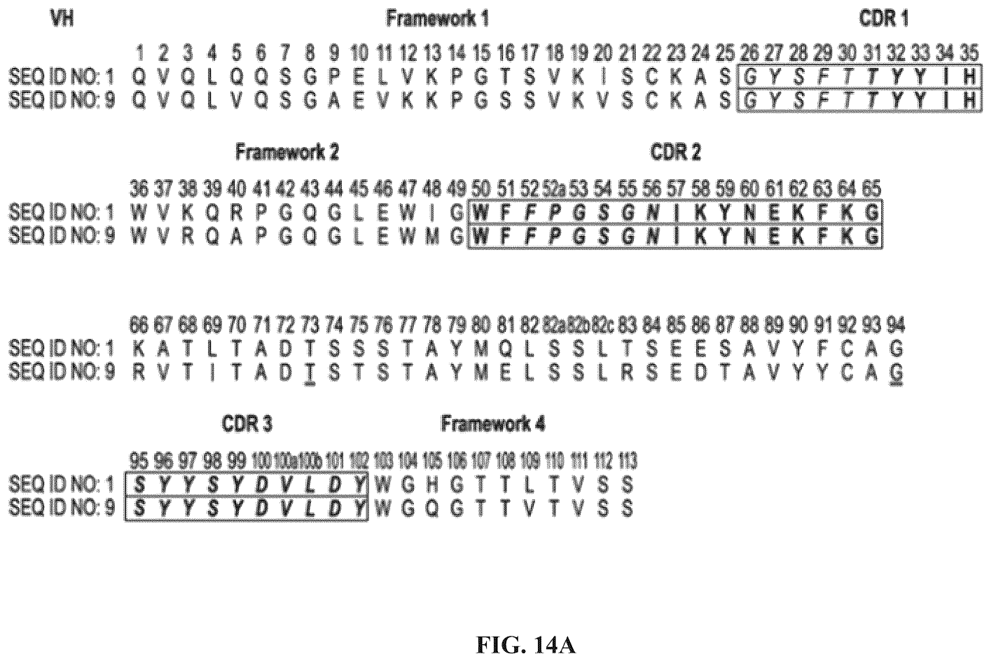

To determine the phenotype of the TCRvβ stimulated CD8− T cells, groups of mice were administered on the following treatments: PBS, α-CD3/IL-2 (one Fc arm contained a CD3 region and the other Fc arm contained a fused IL-2 molecule, 1×1 bispecific), α-vB/IL-2 fusion molecule (1×1 bispecific), or α-vB/CD20 (both Fc arms contained a vβ domain and a CD20 domain, 2×2 bispecific). Blood samples from 7 days and 14 days post injection were collected from the mice. The CD8⁺ T cells from each treatment group were then stained with α-CD44 and α-CD62L. The cells were then gated based on if they had an effector memory T cell phenotype (CD44′ CD62L⁻), a central memory T cell phenotype (CD44⁺ CD62L⁺), or a naïve T cell phenotype (CD44⁻ CD62L⁺) (FIG. 13). In the PBS treatment group, the majority of all CD8⁺ T cells had a naïve phenotype at both 7- and 14-days post-injection. For the α-CD3/IL-2 treatment group, only the 7-day post injection samples were analyzed. At 7 days post injection, the majority of CD8⁺ T cells had an effector memory T cell phenotype. For the α-vB/CD20 treatment group, the majority of CD8⁺ T cells had a naïve phenotype at both 7- and 14-day post injection time points. For the α-vB/IL2 treatment group at 7 days post injection, the majority of CD8⁺ T cells had an effector memory T cell phenotype; however, at 14 days post injection, the majority of CD8⁺ T cells had shifted to having a central memory T cell phenotype. This result confirmed what was seen in vitro with TCRvβ stimulation inducing a CM T cell-like phenotype.

### Example #7: Anti-TCRvβ Antibody Expansion of Polyclonal Effector Memory T Cell Subset

The effects of the anti-TCRvβ antibody on healthy human and cancer patient T cells was assessed in vitro by flow cytometry and NanoString. A anti-TCRvβ antibody murine surrogate was tested as a monotherapy in multiple murine syngeneic tumor models (including PD1-insensitive and refractory), with tumor re-challenge and cellular depletion studies to assess long-term protection and cell-specific activities, respectively. EMT6 tumors were excised for IHC staining and phenotyping of tumor-infiltrating lymphocytes (TILs) using flow cytometry and scRNAseq/TCRseq.

The anti-TCRvβ antibody induced expansion of Vβ6/Vβ10 T cell subsets preferentially in CD8⁺ T cells over CD4⁺ T cells. Compared to controls and anti-CD3 mAbs, anti-TCRvβ antibody-stimulated T cells adopted a novel, highly activated phenotype with markers of both effector and central memory T cells (T_{EM}/T_{CM}). The anti-TCRvβ antibody also boosted the ex vivo expansion of antigen-specific T cells from HPV⁺ individuals (both healthy donor and cancer patient). Consistently across multiple syngeneic murine tumor models, anti-TCRβ antibody murine surrogate monotherapy either eradicated tumors, or led to substantial tumor regressions, anti-TCRvβ antibody murine surrogate-cured mice also demonstrated long-term protection from tumor rechallenge. In vivo anti-tumor activity was shown to be dependent on the accumulation of Vβ6/Vβ10 T cells, and analysis of murine TILs showed expanded Vβ6/Vβ10 T cells were almost exclusively polyclonal T_{EM} or T_{CM} cells, with almost no exhausted or regulatory T cells.

### Example #8: An Exemplary T Cell-Activating Antibody Molecule that Selectively Targets the TCRβ Chain Promotes Antitumor Activity Through Activation and Expansion of a Polyclonal Effector Memory T Cell Subset

Limitations with agents that enhance endogenous T cell responses to cancer, particularly in solid tumors, supports the study of alternative approaches. Directly targeting the variable (V) regions of the T cell receptor (TCR) is an approach to inducing T cell activation. An exemplary anti-TCRvβ antibody molecule, a multispecific molecule containing an anti-TCRVβ6 binding domain and an IL-2 domain (Compound 1), is a bispecific antibody-fusion molecule that selectively activates and expands a subset of human αβ T cells expressing the germline-encoded Vβ6 and Vβ10 TCRs that are enriched in tumor infiltrating lymphocytes. Compound 1 simultaneously engages a non-clonal mode of TCR activation with cytokine co-stimulation.

The effects of Compound 1 on activation and expansion of primary human T cells was assessed in vitro by flow cytometry, homogeneous time-resolved fluorescence, TCRseq. and NanoString. A murine surrogate bispecific antibody (BsAb) of Compound 1 (mCompound 1) was tested in murine syngeneic tumor models with tumor re-challenge and cellular depletion studies to assess potential for long-term protection and cell-specific activities, respectively. EMT6 tumors were excised for IHC staining and phenotyping of tumor-infiltrating lymphocytes (TILs) using flow cytometry and scRNAseq/TCRseq.

In vitro, Compound 1 induced TCR signalling and IL-2R pathway activation in human T cells that preceded expansion of Vβ6/Vβ10 T cell subsets to 80-90% of the T cell compartment. Compared to controls, 80-90% of Compound 1-stimulated human T cells adopted an activated central memory (TCM)-like phenotype. In multiple syngeneic murine tumor models, mCompound 1 monotherapy either eradicated tumors, or led to substantial regressions (60-70r tumor growth inhibition) with long-term protection from tumor rechallenge. In vivo anti-tumor activity was dependent on the accumulation of Vβ T cell subsets, and analysis of TILs showed expanded Vβ T cells were almost exclusively polyclonal effector memory T cells (TEM) or TCM cells with minimal exhausted T cells or Tregs and were associated with a novel gene signature comprising upregulation of memory and effector programs, and downregulation of exhaustion pathways.

Compound 1 is an exemplary anti-TCRvβ bi-specific fusion molecule that selectively binds and activates subsets of the germline TCR repertoire. In vitro. Compound 1 promotes a novel T cell phenotype with hallmarks of both effector and central memory cells, and in vivo mCompound 1 demonstrates potent and durable single-agent anti-tumor activity in several solid tumor models that is dependent on expanded Vβ T cells. The modulation of the tumor microenvironment (TME), striking increase in TCR diversity, and functional immune memory observed in murine models suggests that Compound 1 could remodel the adaptive immune response to solid tumors that are refractory to checkpoint inhibitor therapy, and thus represents a novel therapeutic strategy for patients.

### Example #9: Preclinical Evaluation of an Exemplary Anti-TCR Vβ Targeted Bispecific Antibody Molecule with Potent Anti-Tumor Activity for PD-1 Refractory Solid Tumors

Despite recent advancements with immune checkpoint inhibitors (e.g., anti-PD1 inhibitors) many cancer patients develop treatment resistance, which supports the study of alternative approaches to induce potent and safe anti-tumor T cell responses. Compound 1 is an exemplary anti-TCRvβ bifunctional antibody fusion molecule that selectively activates and expands a sub set of human αβ T cells expressing variable (V) β6 and β10 regions of the T cell receptor (TCR). Compound 1 simultaneously engages a non-clonal mode of TCR activation with cytokine co-stimulation.

The prevalence of Compound 1-targeted Vβ T cells in tumor infiltrating lymphocytes (TILs) from human tumor tissues was investigated by flow cytometry and by interrogating TIL TCRseq data from a large cancer database. The effects of Compound 1 on T cells from healthy donors and cancer patients were assessed in vitro by flow cytometry and NanoString. Using high tumor mutational burden (TMB) and anti-PD1-insensitive murine and human models, anti-tumor activity, mechanism of action, and an enrichment strategy for patient trials were investigated. Further, the pharmacokinetics (PK) and pharmacodynamics (PD) of IV Compound 1 were investigated in Cynomolgus monkeys.

Presence of Compound 1-targeted Vβ T cells were confirmed in tissue from a range of human tumors, and present as 10-12% of TILs. Stimulation of T cells with Compound 1 resulted in potent expansion with approximately 80% adopting a novel memory phenotype, and significant boosting of antigen-specific T cells. In human autologous tumor organoid models. Compound 1 induced potent expansion of TILs and killing of tumors, including several PD1 refractory tumors. Dose-related anti-tumor activity (100% cure rate with a murine surrogate (mCompound 1)) in EMT6-bearing mice correlated with expansion of memory Vβ CD8+ T cells. In Cynomolgus monkeys, IV Compound 1 induced robust expansion of Vβ CD8+ T cells in blood, with limited cytokine release or expansion of Treg. Based on these data, a PK/PD model to simulate human pharmacology is built and a first-in-human trial with an enriched patient population is designed.

Compound 1 is a T cell activator that targets subsets of the germline TCR repertoire that are enriched in TILs. Compound 1 potently expands both naive and antigen-specific human T cells. In PD1 refractory human and murine tumor models with a high TMB, Compound 1 and mCompound 1 induce potent anti-tumor activity as monotherapy, mediated by selective expansion of Vβ CD8+ memory T cells. This pharmacology was translated into monkeys with IV dosed Compound 1.

### Example #10: An Atypical Central-Memory Like Phenotype can be Induced in Human T Cells by Innate TCRαβ Engagement

The αβ T cell receptor (TCR) has immense diversity, primarily for peptide-MHC (pMHC) complexes that clonally interact with complementarity-determining region (CDR)3 motifs assembled by quasi-random somatic gene rearrangement of TCRα and TCRβ gene segments. This adaptive biology is shown to an even greater extent by TCRγδ with its ligands being qualitatively more diverse than pMHC. However, TCRγδ can also function as an innate receptor whereby germline-encoded variable γ residues are sufficient to engage butyrophilin (BTN) or BTN-like (BTNL) that drive a variety of phenotypic outcomes distinct from clonal CDR3-mediated interactions. This broadened perspective on TCR biology has not hitherto been systematically extended to αβ T cells.

A panel of human TCR variable β (TCRVβ) chain targeting antibodies was assembled, and their binding motifs were determined. Using multi-color flow cytometry and single-cell RNA sequencing, the impact of these antibodies on peripheral blood T cells was assessed by comparison with the impacts of anti-CD3E reagents, for example, OKT3.

Antibodies engaging germline-encoded regions of human TCRVβ chains consistently activated primary human T cells towards an atypical central memory (TCM)-like phenotype distinct from those induced by anti-CD3ε stimulation. Although the cells show myriad surface markers associated with chronic stimulation, they are not exhausted but are highly proliferative with strong cytolytic/Tc1 effector profiles, which include, for example, expression of T-bet and IRF4. Strikingly, this phenotype can be induced in cells previously driven toward effector memory (T_{EM}) and effector memory T cells re-expresses CD45RA (T_{EMRA}) states.

In sum, the use of TCRαβ as an innate receptor offers new insight into T cell biology and an approach in which antibody mediated TCR agonism may be relevant to distinct clinical settings.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. Those skilled in the art will recognize, or be able to ascertain using no more than routine experimentation, many equivalents to the specific embodiments of the invention described herein. Such equivalents are intended to be encompassed by the following claims.

What is claimed is:

1.-138. (canceled)

139. A method for inducing expansion of a population of immune cells into an expanded population of immune cells comprising at least 50% central memory (CM) T cells, wherein the method comprises:
contacting a multifunctional molecule to the population of immune cells, wherein the multifunctional molecule comprises a binding domain comprising a T cell receptor beta variable chain (TCRβV) binding moiety; and
wherein the contacting induces expansion of the population of immune cells into the expanded population of immune cells comprising at least 50% CM T cells.

140. The method of claim 139, wherein at least 70% of the expanded population of immune cells are CM T cells.

141. The method of claim 139, wherein the contacting occurs in vitro.

142. The method of claim 141, wherein the multispecific molecule is attached to a solid surface.

143. The method of claim 139, wherein the multifunctional molecule comprises one or more cytokine molecules.

144. The method of claim 143, wherein the one or more cytokine molecules is selected from the group consisting of interleukin-2 (IL-2), interleukin-7 (IL-7), interleukin-12 (IL-12), interleukin-15 (IL-15), interleukin-18 (IL-18), interleukin-21 (IL-21), interferon gamma, and functional fragments or variants thereof.

145. The method of claim 144, wherein binding of the binding domain to the TCRβV and binding of the one or more cytokine molecule promote killing of cancer cells.

146. The method of claim 139, wherein the TCRβV-binding moiety binds to one or more of TCRvβ2, TCRvβ3-1, TCRvβ4-1, TCRvβ4-2, TCRvβ4-3, TCRvβ5-1, TCRvβ5-4, TCRvβ5-5, TCRvβ5-6, TCRvβ5-8, TCRvβ6-1, TCRvβ6-2, TCRvβ6-3, TCRvβ6-4, TCRvβ6-5, TCRvβ6-6, TCRvβ6-8, TCRvβ6-9, TCRvβ7-2, TCRvβ7-3, TCRvβ7-4, TCRvβ7-6, TCRvβ7-7, TCRvβ7-8, TCRvβ7-9, TCRvβ9, TCRvβ10-1, TCRvβ10-2, TCRvβ10-3, TCRvβ11-1, TCRvβ11-2, TCRvβ11-3, TCRvβ12-3, TCRvβ12-4, TCRvβ12-5, TCRvβ13, TCRvβ14, TCRvβ15, TCRvβ16, TCRvβ18, TCRvβ19, TCRvβ20-1, TCRvβ24-1, TCRvβ25-1, TCRvβ27, TCRvβ28, TCRvβ29-1, or TCRvβ30.

147. The method of claim 146, wherein the TCRβV-binding moiety binds to TCRvβ6-5, TCRvβ20-1, TCRvβ12-3/4, TCRvβ5-1, or any combination thereof.

148. The method of claim 139, wherein the at least 50% of the CM T cells are CD95+, CCR7+, CD45RA−, or any combination thereof.

149. The method of claim 139, wherein at least 10% of the CM T cells are IFNγ+ and TNFα+.

150. The method of claim 139, wherein the multifunctional molecule further comprises a linker.

151. The method of claim 139, wherein the TCRβV-binding moiety comprises a Fab, F(ab′)2, Fv, a single chain Fv (scFv), a single domain antibody, a diabody (dAb), or a camelid antibody.

152. The method of claim 143, wherein the multifunctional molecule comprises a first polypeptide and a second polypeptide; wherein the first polypeptide and the second polypeptide are non-contiguous; wherein: (i) the first polypeptide comprises the TCRβV-binding moiety and a first dimerization module linked to the C-terminus of the TCRβV-binding moiety, wherein the TCRβV-binding moiety comprises a first VL and a first VH; and (ii) the second polypeptide comprises a second dimerization module; and wherein the one or more cytokine molecule is covalently linked to the first polypeptide, the second polypeptide, or a combination thereof.

153. The method of claim 139, wherein the contacting comprises administering the multifunctional molecule to a subject in need thereof.

154. The method of claim 153, wherein the subject has a solid tumor that is refractory to a checkpoint inhibitor therapy.

155. The method of claim 154, wherein the checkpoint inhibitor therapy comprises a PD-1 inhibitor therapy, a LAG-3 inhibitor therapy, a CTLA4 inhibitor therapy, or a TIM-3 inhibitor therapy.

156. A method for determining whether an agent is capable of inducing expansion of a population of immune cells into an expanded population of immune cells, wherein the method comprises:
(a) incubating the population of immune cells with the agent;
(b) producing the expanded population of immune cells; and
(c) measuring one or more immune cell markers of the expanded population of immune cells to determine whether at least 50% of the expanded population of immune cells are central memory (CM) T cells.

157. The method of claim 156, wherein the incubating occurs in vitro or ex vivo.

158. The method of claim 156, wherein the one or more immune cell markers comprises TCRβV+, CCR7+, CCR7−, CD45RA−, CD45RA+, CD95+, CD95−, CCR7+ and CD45RA−, CCR7− and CD45RA+, CD95+ and CCR7+, CD95− and CCR7, CD95+ and CD45RA−, CD95− and CD45RA+, CD95+, CCR7+ and CD45RA−, CD95−, CCR7− and CD45RA+, CD38+, CD38, CD25+, CD25−, CD38+ and CD25+, CD38− and CD25−, PD-1+, PD-1−, TIM-3+, TIM-3+, PD-1+ and TIM-3+, PD-1− and TIM-3−, IFNγ+, IFNγ−, TNFα+, TNFα−, IFNγ+ and TNFα+, IFNγ− and TNFα−, CD62L+, CD62L−, CD44+, CD44−, CD62L+ and CD44+, or CD62L− and CD44−.


SEQUENCE LISTING (3669 sequences)

SEQ ID NO: 1 - protein, 119 aa, synthetic construct
QVQLQQSGPELVKPGTSVKISCKASGYSFTTYYIHWVKQRPGQGLEWIGWFFPGSGNIKYNEKFKGKATLTADTSSSTAYMQLSSLTSEESAVYFCAGSYYSYDVLDYWGHGTTLTVSS

SEQ ID NO: 2 - protein, 107 aa, synthetic construct
DILMTQSQKFMSTSLGDRVSVSCKASQNVGINVVWHQQKPGQSPKALIYSSSHRYSGVPDRFTGSGSGTDFTLTINNVQSEDLAEYFCQQFKSYPLTFGAGTKLELK

SEQ ID NO: 3 - protein, 10 aa, synthetic construct
GYSFTTYYIH

SEQ ID NO: 4 - protein, 17 aa, synthetic construct
WFFPGSGNIKYNEKFKG

SEQ ID NO: 5 - protein, 10 aa, synthetic construct
SYYSYDVLDY

SEQ ID NO: 6 - protein, 11 aa, synthetic construct
KASQNVGINVV

SEQ ID NO: 7 - protein, 7 aa, synthetic construct
SSSHRYS

SEQ ID NO: 8 - protein, 9 aa, synthetic construct
QQFKSYPLT

SEQ ID NO: 9 - protein, 119 aa, synthetic construct
QVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSS

SEQ ID NO: 10 - protein, 107 aa, synthetic construct
DIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 11 - protein, 107 aa, synthetic construct
DIQMTQSPSSLSASVGDRVTITCKASQNVGINVVWHQQKPGKVPKALIYSSSHRYSGVPSRFSGSGSGTDFTLTISSLQPEDVATYFCQQFKSYPLTFGQGTKLEIK

SEQ ID NO: 12 - DNA, 357 bp, synthetic construct
  source 1..357: mol_type = other DNA
caggtgcagctggttcagtctggcgccgaagtgaagaaacctggctcctccgtgaaggtgtcctgcaaggcttccggctactccttcaccacctactacatccactgggtccgacaggcccctggacaaggattggaatggatgggctggttcttccccggctccggcaacatcaagtacaacgagaagttcaagggccgcgtgaccatcaccgccgacacctctacctctaccgcctacatggaactgtccagcctgagatctgaggacaccgccgtgtactactgcgccggctcctactactcttacgacgtgctggattactggggccagggcaccacagtgacagtgtcctct

SEQ ID NO: 13 - DNA, 321 bp, synthetic construct
  source 1..321: mol_type = other DNA
gacatccagatgacccagtctccatccttcctgtccgcctctgtgggcgacagagtgaccatcacatgcaaggcctctcagaacgtgggcatcaacgtcgtgtggcaccagcagaagcctggcaaggctcctaaggctctgatctactcctccagccaccggtactctggcgtgccctctagattttccggctctggctctggcaccgagtttaccctgacaatctccagcctgcagcctgaggacttcgccacctacttttgccagcagttcaagagctaccctctgacctttggccagggcaccaagctggaaatcaag

SEQ ID NO: 14 - DNA, 321 bp, synthetic construct
  source 1..321: mol_type = other DNA
gacatccagatgacccagtctccatcctctctgtccgcctctgtgggcgacagagtgaccatcacatgcaaggcctctcagaacgtgggcatcaacgtcgtgtggcaccagcagaaacctggcaaggtgcccaaggctctgatctactcctccagccacagatactccggcgtgccctctagattctccggctctggctctggcaccgactttaccctgacaatctccagcctgcagcctgaggacgtggccacctacttttgccagcagttcaagagctaccctctgacctttggccagggcaccaagctggaaatcaag

SEQ ID NO: 15 - protein, 117 aa, synthetic construct
DVQLVESGGGLVQPGGSRKLSCAASGFTFSNFGMHWVRQAPDKGLEWVAYISSGSSTIYYADTLKGRFTISRDNPKNTLFLQMTSLRSEDTAMYYCARRGEGAMDYWGQGTSVTVSS

SEQ ID NO: 16 - protein, 106 aa, synthetic construct
ENVLTQSPAIMSASLGEKVTMSCRASSSVNYIYWYQQKSDASPKLWIYYTSNLAPGVPTRFSGSGSGNSYSLTISSMEGEDAATYYCQQFTSSPFTFGSGTKLEIK

SEQ ID NO: 17 - protein, 10 aa, synthetic construct
GFTFSNFGMH

SEQ ID NO: 18 - protein, 17 aa, synthetic construct
YISSGSSTIYYADTLKG

SEQ ID NO: 19 - protein, 8 aa, synthetic construct
RGEGAMDY

SEQ ID NO: 20 - protein, 10 aa, synthetic construct
RASSSVNYIY

SEQ ID NO: 21 - protein, 7 aa, synthetic construct
YTSNLAP

SEQ ID NO: 22 - protein, 9 aa, synthetic construct
QQFTSSPFT

SEQ ID NO: 23 - protein, 117 aa, synthetic construct
QVQLVESGGGVVQPGRSLRLSCAASGFTFSNFGMHWVRQAPGKGLEWVAYISSGSSTIYYADTLKGRFTISRDNSKNTLYLQMNSLRAEDTAVYYCARRGEGAMDYWGQGTTVTVSS

SEQ ID NO: 24 - protein, 117 aa, synthetic construct
EVQLVESGGGLVQPGGSLRLSCAASGFTFSNFGMHWVRQAPGKGLEWVSYISSGSSTIYYADTLKGRFTISRDNAKNSLYLQMNSLRAEDTAVYYCARRGEGAMDYWGQGTTVTVSS

SEQ ID NO: 25 - protein, 117 aa, synthetic construct
EVQLVESGGGLVQPGGSLRLSCAASGFTFSNFGMHWVRQAPGKGLEWVSYISSGSSTIYYADTLKGRFTISRDNSKNTLYLQMNSLRAEDTAVYYCARRGEGAMDYWGQGTTVTVSS

SEQ ID NO: 26 - protein, 106 aa, synthetic construct
DNQLTQSPSFLSASVGDRVTITCRASSSVNYIYWYQQKPGKAPKLLIYYTSNLAPGVPSRFSGSGSGNEYTLTISSLQPEDFATYYCQQFTSSPFTFGQGTKLEIK

SEQ ID NO: 27 - protein, 106 aa, synthetic construct
DNQLTQSPSSLSASVGDRVTITCRASSSVNYIYWYQQKPGKAPKLLIYYTSNLAPGVPSRFSGSGSGNDYTLTISSLQPEDFATYYCQQFTSSPFTFGQGTKLEIK

SEQ ID NO: 28 - protein, 106 aa, synthetic construct
ENVLTQSPATLSVSPGERATLSCRASSSVNYIYWYQQKPGQAPRLLIYYTSNLAPGIPARFSGSGSGNEYTLTISSLQSEDFAVYYCQQFTSSPFTFGQGTKLEIK

SEQ ID NO: 29 - protein, 105 aa, synthetic construct
QNVLTQPPSASGTPGQRVTISCRASSSVNYIYWYQQLPGTAPKLLIYYTSNLAPGVPDRFSGSGSGNSYSLAISGLRSEDEADYYCQQFTSSPFTFGTGTKVTVL

SEQ ID NO: 30 - protein, 105 aa, synthetic construct
SNELTQPPSVSVSPGQTARITCRASSSVNYIYWYQQKSGQAPVLVIYYTSNLAPGIPERFSGSGSGNMYTLTISGAQVEDEADYYCQQFTSSPFTFGTGTKVTVL

SEQ ID NO: 31 - DNA, 351 bp, synthetic construct
  source 1..351: mol_type = other DNA
gaggtgcagctggttgaatctggcggaggattggttcagcctggcggctctctgagactgtcttgtgccgcttctggcttcaccttctccaacttcggcatgcactgggtccgacaggcccctggaaaaggactggaatgggtgtcctacatctcctccggctcctccaccatctactacgctgacaccctgaagggcagattcaccatctctcgggacaacgccaagaactccctgtacctgcagatgaacagcctgagagccgaggacaccgccgtgtactactgtgctagaagaggcgagggcgccatggattattggggccagggaaccacagtgaccgtgtctagc

SEQ ID NO: 32 - DNA, 351 bp, synthetic construct
  source 1..351: mol_type = other DNA
gaggtgcagctggttgaatctggcggaggattggttcagcctggcggctctctgagactgtcttgtgccgcttctggcttcaccttctccaacttcggcatgcactgggtccgacaggcccctggaaaaggactggaatgggtgtcctacatctcctccggctcctccaccatctactacgctgacaccctgaagggcagattcaccatcagccgggacaactccaagaacaccctgtacctgcagatgaactccctgagagccgaggacaccgccgtgtactactgtgctagaagaggcgagggcgccatggattattggggccagggaaccacagtgaccgtgtctagc

SEQ ID NO: 33 - DNA, 351 bp, synthetic construct
  source 1..351: mol_type = other DNA
caggtgcagctggtggaatctggtggcggagttgtgcagcctggcagatccctgagactgtcttgtgccgcctctggcttcaccttctccaacttcggcatgcactgggtccgacaggcccctggaaaaggattggagtgggtcgcctacatctcctccggctcctccaccatctactacgctgacaccctgaagggcagattcaccatcagccgggacaactccaagaacaccctgtacctgcagatgaactccctgagagccgaggacaccgccgtgtactactgtgctagaagaggcgagggcgccatggattattggggccagggaaccacagtgaccgtgtctagc

SEQ ID NO: 34 - DNA, 318 bp, synthetic construct
  source 1..318: mol_type = other DNA
gataaccagctgacccagtctcctagcttcctgtctgcctctgtgggcgacagagtgacaattacctgccgggcctcctcctccgtgaactacatctactggtatcagcagaagcccggcaaggcccctaagctgctgatctactacacctccaatctggcccctggcgtgccctctagattttccggatctggctccggcaacgagtataccctgacaatctccagcctgcagcctgaggacttcgccacctactactgccagcagttcacctcctctccattcacctttggccagggcaccaagctggaaatcaaa

SEQ ID NO: 35 - DNA, 317 bp, synthetic construct
  source 1..317: mol_type = other DNA
ataaccagctgacccagtctccttccagcctgtctgcttctgtgggcgacagagtgacaattacctgccgggcctcctcctccgtgaactacatctactggtatcagcagaagcccggcaaggcccctaagctgctgatctactacacctccaatctggcccctggcgtgccctctagattttccggatctggctccggcaacgactataccctgacaatctccagcctgcagcctgaggacttcgccacctactactgccagcagttcacctcctctccattcacctttggccagggcaccaagctggaaatcaaa

SEQ ID NO: 36 - DNA, 318 bp, synthetic construct
  source 1..318: mol_type = other DNA
gagaatgtgctgacccagtctcctgccacactgtctgttagccctggcgagagagctaccctgagctgcagagcctcttcctccgtgaactacatctactggtatcagcagaagcccggccaggctcctagactgctgatctactacacctccaatctggcccctggcatccctgccagattttccggatctggctccggcaacgagtataccctgaccatctccagcctgcagtccgaggactttgctgtgtactattgccagcagttcacaagcagccctttcacctttggccagggcaccaagctggaaatcaaa

SEQ ID NO: 37 - DNA, 315 bp, synthetic construct
  source 1..315: mol_type = other DNA
cagaatgtgctgacccaacctccttccgcctctggcacacctggacagagagtgacaatctcctgccgggcctcctcctccgtgaactacatctactggtatcagcagctgcccggcaccgctcctaaactgctgatctactacacctccaatctggcccctggcgtgcccgatagattttccggatctggctccggcaactcctacagcctggctatctctggcctgagatctgaggacgaggccgactactactgccagcagttcacctcctctccattcacctttggcaccggcaccaaagtgacagttctt

SEQ ID NO: 38 - DNA, 315 bp, synthetic construct
  source 1..315: mol_type = other DNA
tctaatgagctgacccagcctccttccgtgtccgtgtctcctggacagaccgccagaattacctgccgggcctcctcctccgtgaactacatctactggtatcagcagaagtccggccaggctcctgtgctcgtgatctactacacctccaatctggcccctggcatccctgagagattctccggatctggctccggcaacatgtacaccctgaccatctctggcgcccaggtggaagatgaggccgactactactgccagcagttcacctcctctccattcacctttggcaccggcaccaaagtgacagttctt

SEQ ID NO: 39 - protein, 107 aa, synthetic construct [sequence omitted]

SEQ ID NO: 40 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 41 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 344 bp, Homo sapiens
  source 1..344: mol_type = genomic DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttactc

SEQ ID NO: 44 - protein, 114 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSY

SEQ ID NO: 45 - protein, 5 aa, synthetic construct
TYYIH

SEQ ID NO: 46 - protein, 17 aa, synthetic construct
WFFPGSGNIKYNEKFKG

SEQ ID NO: 47 - protein, 10 aa, synthetic construct
SYYSYDVLDY

SEQ ID NO: 48 - protein, 7 aa, synthetic construct
GYSFTTY

SEQ ID NO: 49 - protein, 6 aa, synthetic construct
FPGSGN

SEQ ID NO: 50 - protein, 10 aa, synthetic construct
SYYSYDVLDY

SEQ ID NO: 51 - protein, 11 aa, synthetic construct
KASQNVGINVV

SEQ ID NO: 52 - protein, 7 aa, synthetic construct
SSSHRYS

SEQ ID NO: 53 - protein, 9 aa, synthetic construct
QQFKSYPLT

SEQ ID NO: 54 - protein, 11 aa, synthetic construct
KASQNVGINVV

SEQ ID NO: 55 - protein, 7 aa, synthetic construct
SSSHRYS

SEQ ID NO: 56 - protein, 9 aa, synthetic construct
QQFKSYPLT

SEQ ID NO: 57 - protein, 5 aa, synthetic construct
NFGMH

SEQ ID NO: 58 - protein, 17 aa, synthetic construct
YISSGSSTIYYADTLKG

SEQ ID NO: 59 - protein, 8 aa, synthetic construct
RGEGAMDY

SEQ ID NO: 60 - protein, 7 aa, synthetic construct
GFTFSNF

SEQ ID NO: 61 - protein, 6 aa, synthetic construct
SSGSST

SEQ ID NO: 62 - protein, 8 aa, synthetic construct
RGEGAMDY

SEQ ID NO: 63 - protein, 10 aa, synthetic construct
RASSSVNYIY

SEQ ID NO: 64 - protein, 7 aa, synthetic construct
YTSNLAP

SEQ ID NO: 65 - protein, 9 aa, synthetic construct
QQFTSSPFT

SEQ ID NO: 66 - protein, 10 aa, synthetic construct
RASSSVNYIY

SEQ ID NO: 67 - protein, 7 aa, synthetic construct
YTSNLAP

SEQ ID NO: 68 - protein, 9 aa, synthetic construct
QQFTSSPFT

SEQ ID NO: 69 - protein, 592 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 70 - protein, 492 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 71 - protein, 479 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 72 - protein, 234 aa, synthetic construct
METDTLLLWVLLLWVPGSTGDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 453 aa, synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 74 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 75 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 76 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 77-79 - skipped (3 entries)

SEQ ID NO: 80 - protein, 246 aa, synthetic construct
QVQLVQSGAEVKKPGSSVKVSCKASGTDFKLTYIHWVRQAPGQGLEWMGRVSPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 81 - protein, 107 aa, synthetic construct
DIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 82 - protein, 119 aa, synthetic construct
QVQLVQSGAEVKKPGSSVKVSCKASGTDFKLTYIHWVRQAPGQGLEWMGRVSPGSGNTKYNEKFKGRVTITADTSTSTAYMELSSLRSEDTAVYYCAGSYYSYDVLDYWGQGTTVTVSS

SEQ ID NO: 83 - protein, 246 aa, synthetic construct
QVQLVQSGAEVKKPGSSVKVSCKASGTDFDKIYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 84 - protein, 107 aa, synthetic construct; identical to SEQ ID NO: 81

SEQ ID NO: 85 - protein, 119 aa, synthetic construct
QVQLVQSGAEVKKPGSSVKVSCKASGTDFDKIYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 86 - protein, 246 aa, synthetic construct
QVQLVQSGAEVKKPGSSVKVSCKASGHDFRDFYIHWVRQAPGQGLEWMGRVYPGSGSYRYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 87 - protein, 107 aa, synthetic construct
DIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 88 - protein, 119 aa, synthetic construct
QVQLVQSGAEVKKPGSSVKVSCKASGHDFRDFYIHWVRQAPGQGLEWMGRVYPGSGSYRYNEKFKGRVTITADTSTSTAYMELSSLRSEDTAVYYCAGSYYSYDVLDYWGQGTTVTVSS

SEQ ID NO: 89 - protein, 246 aa, synthetic construct
QVQLVQSGAEVKKPGSSVKVSCKASGHDFKLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 90 - protein, 107 aa, synthetic construct
DIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 91 - protein, 119 aa, synthetic construct
QVQLVQSGAEVKKPGSSVKVSCKASGHDFKLTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 92 - protein, 246 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVENKVAWHQQKPGKAPKALIYSSSHRYKGVPSRFSGSGSGTEFTLTISSLQPEDFATYFCQQFKSYPLTFGQGTKLEIK

SEQ ID NO: 93 - protein, 107 aa, synthetic construct
DIQMTQSPSFLSASVGDRVTITCKASQNVENKVAWHQQKPGKAPKALIYSSSHRYKGVPSRFSGSGSGTEFTLTISSLQPEDFATYFCQQFKSYPLTFGQGTKLEIK

SEQ ID NO: 94 - protein, 119 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSS

SEQ ID NO: 95 - protein, 246 aa, synthetic construct
QVQLVQSGAEVKKPGSSVKVSCKASGTDFDKIYIHWVRQAPGQGLEWMGRIFAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 96 - protein, 107 aa, synthetic construct; identical to SEQ ID NO: 87

SEQ ID NO: 97 - protein, 119 aa, synthetic construct
QVQLVQSGAEVKKPGSSVKVSCKASGTDFDKIYIHWVRQAPGQGLEWMGRIFAGSGNTKYNEKFKGRVTITADTSTSTAYMELSSLRSEDTAVYYCAGSYYSYDVLDYWGQGTTVTVSS

SEQ ID NO: 98 - protein, 246 aa, synthetic construct
QVQLVQSGAEVKKPGSSVKVSCKASGHDFDKFYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYSGVPSRFSGSGSGTEFTLTISSLQPEDFATYFCQQFKSYPLTFGQGTKLEIK

SEQ ID NO: 99 - protein, 107 aa, synthetic construct
DIQMTQSPSFLSASVGDRVTITCKASQNVGNRVAWYQQKPGKAPKALIYSSSHRYSGVPSRFSGSGSGTEFTLTISSLQPEDFATYFCQQFKSYPLTFGQGTKLEIK

SEQ ID NO: 100 - protein, 119 aa, synthetic construct
QVQLVQSGAEVKKPGSSVKVSCKASGHDFDKFYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 101 - protein, 247 aa, synthetic construct
QVQLVQSGAEVKKPGSSVKVSCKASGHDFDKFYIHWVRQAPGQGLEWMGRIFAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIKS

SEQ ID NO: 102 - protein, 107 aa, synthetic construct
DIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 103 - protein, 119 aa, synthetic construct
QVQLVQSGAEVKKPGSSVKVSCKASGHDFDKFYIHWVRQAPGQGLEWMGRIFAGSGNVKYNEKFKGRVTITADTSTSTAYMELSSLRSEDTAVYYCAGSYYSYDVLDYWGQGTTVTVSS

SEQ ID NO: 104 - protein, 246 aa, synthetic construct
QVQLVQSGAEVKKPGSSVKVSCKASGTDFKLTYIHWVRQAPGQGLEWMGRVSPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 105 - protein, 107 aa, synthetic construct; identical to SEQ ID NO: 102

SEQ ID NO: 106 - protein, 119 aa, synthetic construct
QVQLVQSGAEVKKPGSSVKVSCKASGTDFKLTYIHWVRQAPGQGLEWMGRVSPGSGNVKYNEKFKGRVTITADTSTSTAYMELSSLRSEDTAVYYCAGSYYSYDVLDYWGQGTTVTVSS

SEQ ID NO: 107 - protein, 246 aa, synthetic construct
QVQLVQSGAEVKKPGSSVKVSCKASGTDFDKIYIHWVRQAPGQGLEWMGRVSAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 108 - protein, 107 aa, synthetic construct
DIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 109 - protein, 119 aa, synthetic construct
QVQLVQSGAEVKKPGSSVKVSCKASGTDFDKIYIHWVRQAPGQGLEWMGRVSAGSGNTKYNEKFKGRVTITADTSTSTAYMELSSLRSEDTAVYYCAGSYYSYDVLDYWGQGTTVTVSS

SEQ ID NO: 110 - protein, 246 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 111 - protein, 107 aa, synthetic construct; identical to SEQ ID NO: 90

SEQ ID NO: 112 - protein, 119 aa, synthetic construct; identical to SEQ ID NO: 94

SEQ ID NO: 113 - protein, 246 aa, synthetic construct
QVQLVQSGAEVKKPGSSVKVSCKASGTDFDKIYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 114 - protein, 107 aa, synthetic construct; identical to SEQ ID NO: 87

SEQ ID NO: 115 - protein, 119 aa, synthetic construct
QVQLVQSGAEVKKPGSSVKVSCKASGTDFDKIYIHWVRQAPGQGLEWMGRISAGSGNTKYNEKFKGRVTITADTSTSTAYMELSSLRSEDTAVYYCAGSYYSYDVLDYWGQGTTVTVSS

SEQ ID NO: 116 - protein, 246 aa, synthetic construct
QVQLVQSGAEVKKPGSSVKVSCKASGTDFRLTYIHWVRQAPGQGLEWMGRVSPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNKVAWHQQKPGKAPKALIYSSSHRYKGVPSRFSGSGSGTEFTLTISSLQPEDFATYFCQQFKSYPLTFGQGTKLEIK

SEQ ID NO: 117 - protein, 107 aa, synthetic construct
DIQMTQSPSFLSASVGDRVTITCKASQNVDNKVAWHQQKPGKAPKALIYSSSHRYKGVPSRFSGSGSGTEFTLTISSLQPEDFATYFCQQFKSYPLTFGQGTKLEIK

SEQ ID NO: 118 - protein, 119 aa, synthetic construct
QVQLVQSGAEVKKPGSSVKVSCKASGTDFRLTYIHWVRQAPGQGLEWMGRVSPGSGNTKYNEKFKGRVTITADTSTSTAYMELSSLRSEDTAVYYCAGSYYSYDVLDYWGQGTTVTVSS

SEQ ID NO: 119 - protein, 246 aa, synthetic construct
QVQLVQSGAEVKKPGSSVKVSCKASGGTFRLTYIHWVRQAPGQGLEWMGRVYPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 120 - protein, 107 aa, synthetic construct; identical to SEQ ID NO: 87

SEQ ID NO: 121 - protein, 119 aa, synthetic construct
QVQLVQSGAEVKKPGSSVKVSCKASGGTFRLTYIHWVRQAPGQGLEWMGRVYPGSGNTKYNEKFKGRVTITADTSTSTAYMELSSLRSEDTAVYYCAGSYYSYDVLDYWGQGTTVTVSS

SEQ ID NO: 122 - protein, 246 aa, synthetic construct
QVQLVQSGAEVKKPGSSVKVSCKASGTDFRLTYIHWVRQAPGQGLEWMGRIFPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 123 - protein, 107 aa, synthetic construct; identical to SEQ ID NO: 87

SEQ ID NO: 124 - protein, 119 aa, synthetic construct
QVQLVQSGAEVKKPGSSVKVSCKASGTDFRLTYIHWVRQAPGQGLEWMGRIFPGSGNTKYNEKFKGRVTITADTSTSTAYMELSSLRSEDTAVYYCAGSYYSYDVLDYWGQGTTVTVSS

SEQ ID NO: 125 - protein, 246 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 126 - protein, 107 aa, synthetic construct; identical to SEQ ID NO: 81

SEQ ID NO: 127 - protein, 119 aa, synthetic construct; identical to SEQ ID NO: 94

SEQ ID NO: 128 - protein, 246 aa, synthetic construct
QVQLVQSGAEVKKPGSSVKVSCKASGGTFRLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 129 - protein, 107 aa, synthetic construct; identical to SEQ ID NO: 102

SEQ ID NO: 130 - protein, 119 aa, synthetic construct
QVQLVQSGAEVKKPGSSVKVSCKASGGTFRLTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 131 - protein, 246 aa, synthetic construct
QVQLVQSGAEVKKPGSSVKVSCKASGGTFDKTYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 132 - protein, 107 aa, synthetic construct; identical to SEQ ID NO: 87

SEQ ID NO: 133 - protein, 119 aa, synthetic construct
QVQLVQSGAEVKKPGSSVKVSCKASGGTFDKTYIHWVRQAPGQGLEWMGRISAGSGNTKYNEKFKGRVTITADTSTSTAYMELSSLRSEDTAVYYCAGSYYSYDVLDYWGQGTTVTVSS

SEQ ID NO: 134 - protein, 246 aa, synthetic construct
QVQLVQSGAEVKKPGSSVKVSCKASGHDFDKFYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 135 - protein, 107 aa, synthetic construct; identical to SEQ ID NO: 87

SEQ ID NO: 136 - protein, 119 aa, synthetic construct
QVQLVQSGAEVKKPGSSVKVSCKASGHDFDKFYIHWVRQAPGQGLEWMGRISAGSGNTKYNEKFKGRVTITADTSTSTAYMELSSLRSEDTAVYYCAGSYYSYDVLDYWGQGTTVTVSS

SEQ ID NO: 137 - protein, 246 aa, synthetic construct
QVQLVQSGAEVKKPGSSVKVSCKASGTDFKLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNKVAWHQQKPGKAPKALIYSSSHRYKGVPSRFSGSGSGTEFTLTISSLQPEDFATYFCQQFKSYPLTFGQGTKLEIK

SEQ ID NO: 138 - protein, 107 aa, synthetic construct; identical to SEQ ID NO: 117

SEQ ID NO: 139 - protein, 119 aa, synthetic construct
QVQLVQSGAEVKKPGSSVKVSCKASGTDFKLTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 140 - protein, 246 aa, synthetic construct
QVQLVQSGAEVKKPGSSVKVSCKASGHDFRLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 141 - protein, 107 aa, synthetic construct
DIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 142 - protein, 119 aa, synthetic construct
QVQLVQSGAEVKKPGSSVKVSCKASGHDFRLTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 143 - protein, 246 aa, synthetic construct
QVQLVQSGAEVKKPGSSVKVSCKASGHDFHLWYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNKVAWHQQKPGKAPKALIYSSSHRYKGVPSRFSGSGSGTEFTLTISSLQPEDFATYFCQQFKSYPLTFGQGTKLEIK

SEQ ID NO: 144 - protein, 107 aa, synthetic construct; identical to SEQ ID NO: 117

SEQ ID NO: 145 - protein, 119 aa, synthetic construct
QVQLVQSGAEVKKPGSSVKVSCKASGHDFHLWYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 146 - protein, 246 aa, synthetic construct
QVQLVQSGAEVKKPGSSVKVSCKASGHDFHLWYIHWVRQAPGQGLEWMGRVFAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDKVAWYQQKPGKAPKALIYSSSHRYKGVPSRFSGSGSGTEFTLTISSLQPEDFATYFCQQFKSYPLTFGQGTKLEIK

SEQ ID NO: 147 - protein, 107 aa, synthetic construct
DIQMTQSPSFLSASVGDRVTITCKASQNVEDKVAWYQQKPGKAPKALIYSSSHRYKGVPSRFSGSGSGTEFTLTISSLQPEDFATYFCQQFKSYPLTFGQGTKLEIK

SEQ ID NO: 148 - protein, 119 aa, synthetic construct
QVQLVQSGAEVKKPGSSVKVSCKASGHDFHLWYIHWVRQAPGQGLEWMGRVFAGSGNTKYNEKFKGRVTITADTSTSTAYMELSSLRSEDTAVYYCAGSYYSYDVLDYWGQGTTVTVSS

SEQ ID NO: 149 - protein, 246 aa, synthetic construct
QVQLVQSGAEVKKPGSSVKVSCKASGTDFKLTYIHWVRQAPGQGLEWMGRIFPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 150 - protein, 107 aa, synthetic construct; identical to SEQ ID NO: 87

SEQ ID NO: 151 - protein, 119 aa, synthetic construct
QVQLVQSGAEVKKPGSSVKVSCKASGTDFKLTYIHWVRQAPGQGLEWMGRIFPGSGNTKYNEKFKGRVTITADTSTSTAYMELSSLRSEDTAVYYCAGSYYSYDVLDYWGQGTTVTVSS

SEQ ID NO: 152 - skipped

SEQ ID NO: 153 - protein, 246 aa, synthetic construct
QVQLVQSGAEVKKPGSSVKVSCKASGTDFKLTYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 154 - protein, 107 aa, synthetic construct; identical to SEQ ID NO: 108

SEQ ID NO: 155 - protein, 119 aa, synthetic construct
QVQLVQSGAEVKKPGSSVKVSCKASGTDFKLTYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 156 - protein, 246 aa, synthetic construct
QVQLVQSGAEVKKPGSSVKVSCKASGTDFKLTYIHWVRQAPGQGLEWMGRISPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 157 - protein, 107 aa, synthetic construct; identical to SEQ ID NO: 102

SEQ ID NO: 158 - protein, 119 aa, synthetic construct
QVQLVQSGAEVKKPGSSVKVSCKASGTDFKLTYIHWVRQAPGQGLEWMGRISPGSGNTKYNEKFKGRVTITADTSTSTAYMELSSLRSEDTAVYYCAGSYYSYDVLDYWGQGTTVTVSS

SEQ ID NO: 159 - protein, 246 aa, synthetic construct
QVQLVQSGAEVKKPGSSVKVSCKASGHDFHLWYIHWVRQAPGQGLEWMGRISPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWHQQKPGKAPKALIYSSSHRYKGVPSRFSGSGSGTEFTLTISSLQPEDFATYFCQQFKSYPLTFGQGTKLEIK

SEQ ID NO: 160 - protein, 107 aa, synthetic construct
DIQMTQSPSFLSASVGDRVTITCKASQNVGDRVAWHQQKPGKAPKALIYSSSHRYKGVPSRFSGSGSGTEFTLTISSLQPEDFATYFCQQFKSYPLTFGQGTKLEIK

SEQ ID NO: 161 - protein, 119 aa, synthetic construct
QVQLVQSGAEVKKPGSSVKVSCKASGHDFHLWYIHWVRQAPGQGLEWMGRISPGSGNVKYNEKFKGRVTITADTSTSTAYMELSSLRSEDTAVYYCAGSYYSYDVLDYWGQGTTVTVSS

SEQ ID NO: 162 - protein, 246 aa, synthetic construct
QVQLVQSGAEVKKPGSSVKVSCKASGHDFKLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 163 - protein, 107 aa, synthetic construct; identical to SEQ ID NO: 87

SEQ ID NO: 164 - protein, 119 aa, synthetic construct; identical to SEQ ID NO: 91

SEQ ID NO: 165 - protein, 246 aa, synthetic construct
QVQLVQSGAEVKKPGSSVKVSCKASGTDFHLWYIHWVRQAPGQGLEWMGRVFAGSGSYRYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 166 - protein, 107 aa, synthetic construct; identical to SEQ ID NO: 87

SEQ ID NO: 167 - protein, 119 aa, synthetic construct
QVQLVQSGAEVKKPGSSVKVSCKASGTDFHLWYIHWVRQAPGQGLEWMGRVFAGSGSYRYNEKFKGRVTITADTSTSTAYMELSSLRSEDTAVYYCAGSYYSYDVLDYWGQGTTVTVSS

SEQ ID NO: 168 - protein, 246 aa, synthetic construct
QVQLVQSGAEVKKPGSSVKVSCKASGTDFDKIYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 169 - protein, 107 aa, synthetic construct; identical to SEQ ID NO: 141

SEQ ID NO: 170 - protein, 119 aa, synthetic construct; identical to SEQ ID NO: 115

SEQ ID NO: 171 - protein, 246 aa, synthetic construct
QVQLVQSGAEVKKPGSSVKVSCKASGTDFKLTYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 172 - protein, 107 aa, synthetic construct; identical to SEQ ID NO: 81

SEQ ID NO: 173 - protein, 119 aa, synthetic construct
QVQLVQSGAEVKKPGSSVKVSCKASGTDFKLTYIHWVRQAPGQGLEWMGRISAGSGNTKYNEKFKGRVTITADTSTSTAYMELSSLRSEDTAVYYCAGSYYSYDVLDYWGQGTTVTVSS

SEQ ID NO: 174 - protein, 246 aa, synthetic construct
QVQLVQSGAEVKKPGSSVKVSCKASGTDFRLTYIHWVRQAPGQGLEWMGRISPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 175 - protein, 107 aa, synthetic construct; identical to SEQ ID NO: 108

SEQ ID NO: 176 - protein, 119 aa, synthetic construct
QVQLVQSGAEVKKPGSSVKVSCKASGTDFRLTYIHWVRQAPGQGLEWMGRISPGSGNTKYNEKFKGRVTITADTSTSTAYMELSSLRSEDTAVYYCAGSYYSYDVLDYWGQGTTVTVSS

SEQ ID NO: 177 - protein, 246 aa, synthetic construct
QVQLVQSGAEVKKPGSSVKVSCKASGHDFDKTYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 178 - protein, 107 aa, synthetic construct; identical to SEQ ID NO: 81

SEQ ID NO: 179 - protein, 119 aa, synthetic construct
QVQLVQSGAEVKKPGSSVKVSCKASGHDFDKTYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 180 - protein, 246 aa, synthetic construct
QVQLVQSGAEVKKPGSSVKVSCKASGHDFKLTYIHWVRQAPGQGLEWMGRVSPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWHQQKPGKAPKALIYSSSHRYKGVPSRFSGSGSGTEFTLTISSLQPEDFATYFCQQFKSYPLTFGQGTKLEIK

SEQ ID NO: 181 - protein, 107 aa, synthetic construct
DIQMTQSPSFLSASVGDRVTITCKASQNVEDRVAWHQQKPGKAPKALIYSSSHRYKGVPSRFSGSGSGTEFTLTISSLQPEDFATYFCQQFKSYPLTFGQGTKLEIK

SEQ ID NO: 182 - protein, 119 aa, synthetic construct
QVQLVQSGAEVKKPGSSVKVSCKASGHDFKLTYIHWVRQAPGQGLEWMGRVSPGSGNTKYNEKFKGRVTITADTSTSTAYMELSSLRSEDTAVYYCAGSYYSYDVLDYWGQGTTVTVSS

SEQ ID NO: 183 - protein, 246 aa, synthetic construct
QVQLVQSGAEVKKPGSSVKVSCKASGHDFDKTYIHWVRQAPGQGLEWMGRIYPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 184 - protein, 107 aa, synthetic construct; identical to SEQ ID NO: 141

SEQ ID NO: 185 - protein, 119 aa, synthetic construct
QVQLVQSGAEVKKPGSSVKVSCKASGHDFDKTYIHWVRQAPGQGLEWMGRIYPGSGNVKYNEKFKGRVTITADTSTSTAYMELSSLRSEDTAVYYCAGSYYSYDVLDYWGQGTTVTVSS

SEQ ID NO: 186 - protein, 246 aa, synthetic construct
QVQLVQSGAEVKKPGSSVKVSCKASGTDFDKTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 187 - protein, 107 aa, synthetic construct; identical to SEQ ID NO: 87

SEQ ID NO: 188 - protein, 119 aa, synthetic construct
QVQLVQSGAEVKKPGSSVKVSCKASGTDFDKTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 189 - protein, 246 aa, synthetic construct
QVQLVQSGAEVKKPGSSVKVSCKASGTDFDKIYIHWVRQAPGQGLEWMGRISAGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 190 - protein, 107 aa, synthetic construct; identical to SEQ ID NO: 87

SEQ ID NO: 191 - protein, 119 aa, synthetic construct
QVQLVQSGAEVKKPGSSVKVSCKASGTDFDKIYIHWVRQAPGQGLEWMGRISAGSGNIKYNEKFKGRVTITADTSTSTAYMELSSLRSEDTAVYYCAGSYYSYDVLDYWGQGTTVTVSS

SEQ ID NO: 192 - protein, 246 aa, synthetic construct
QVQLVQSGAEVKKPGSSVKVSCKASGTDFDKIYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 193 - protein, 107 aa, synthetic construct; identical to SEQ ID NO: 102

SEQ ID NO: 194 - protein, 119 aa, synthetic construct; identical to SEQ ID NO: 85

SEQ ID NO: 195 - protein, 246 aa, synthetic construct
QVQLVQSGAEVKKPGSSVKVSCKASGGTFKLTYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 196 - protein, 107 aa, synthetic construct; identical to SEQ ID NO: 87

SEQ ID NO: 197 - protein, 119 aa, synthetic construct
QVQLVQSGAEVKKPGSSVKVSCKASGGTFKLTYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 198 - protein, 246 aa, synthetic construct
QVQLVQSGAEVKKPGSSVKVSCKASGTDFKLTYIHWVRQAPGQGLEWMGRISPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWHQQKPGKAPKALIYSSSHRYKGVPSRFSGSGSGTEFTLTISSLQPEDFATYFCQQFKSYPLTFGQGTKLEIK

SEQ ID NO: 199 - protein, 107 aa, synthetic construct
DIQMTQSPSFLSASVGDRVTITCKASQNVDNRVAWHQQKPGKAPKALIYSSSHRYKGVPSRFSGSGSGTEFTLTISSLQPEDFATYFCQQFKSYPLTFGQGTKLEIK

SEQ ID NO: 200 - protein, 119 aa, synthetic construct
QVQLVQSGAEVKKPGSSVKVSCKASGTDFKLTYIHWVRQAPGQGLEWMGRISPGSGNVKYNEKFKGRVTITADTSTSTAYMELSSLRSEDTAVYYCAGSYYSYDVLDYWGQGTTVTVSS

SEQ ID NO: 201 - protein, 246 aa, synthetic construct
QVQLVQSGAEVKKPGSSVKVSCKASGHDFDKFYIHWVRQAPGQGLEWMGRVSAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 202 - protein, 107 aa, synthetic construct; identical to SEQ ID NO: 90

SEQ ID NO: 203 - protein, 119 aa, synthetic construct
QVQLVQSGAEVKKPGSSVKVSCKASGHDFDKFYIHWVRQAPGQGLEWMGRVSAGSGNTKYNEKFKGRVTITADTSTSTAYMELSSLRSEDTAVYYCAGSYYSYDVLDYWGQGTTVTVSS

SEQ ID NO: 204 - protein, 246 aa, synthetic construct
QVQLVQSGAEVKKPGSSVKVSCKASGTDFDKFYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVVWYQQKPGKAPKALIYSSSHRYKGVPSRFSGSGSGTEFTLTISSLQPEDFATYFCQQFKSYPLTFGQGTKLEIK

SEQ ID NO: 205 - protein, 119 aa, synthetic construct
QVQLVQSGAEVKKPGSSVKVSCKASGTDFDKFYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 206 - protein, 246 aa, synthetic construct
QVQLVQSGAEVKKPGSSVKVSCKASGYSFTTYYIHWVRQAPGQGLEWMGWFSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 207 - protein, 119 aa, synthetic construct
QVQLVQSGAEVKKPGSSVKVSCKASGYSFTTYYIHWVRQAPGQGLEWMGWFSAGSGNTKYNEKFKGRVTITADTSTSTAYMELSSLRSEDTAVYYCAVSYYSYDVLDYWGQGTTVTVSS

SEQ ID NO: 208 - protein, 246 aa, synthetic construct
QVQLVQSGAEVKKPGSSVKVSCKASGYSFTTYYIHWVRQAPGQGLEWMGWFSAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 209 - protein, 119 aa, synthetic construct
QVQLVQSGAEVKKPGSSVKVSCKASGYSFTTYYIHWVRQAPGQGLEWMGWFSAGSGNTKYNEKFKGRVTITADTSTSTAYMELSSLRSEDTAVYYCAGSYYSYDVLDYWGQGTTVTVSS

SEQ ID NO: 210 - protein, 246 aa, synthetic construct
QVQLVQSGAEVKKPGSSVKVSCKASGYSFTTYYIHWVRQAPGQGLEWMGWFFPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 211 - protein, 119 aa, synthetic construct
QVQLVQSGAEVKKPGSSVKVSCKASGYSFTTYYIHWVRQAPGQGLEWMGWFFPGSGNTKYNEKFKGRVTITADTSTSTAYMELSSLRSEDTAVYYCAGSYYSYDVLDYWGQGTTVTVSS

SEQ ID NO: 212 - protein, 246 aa, synthetic construct
QVQLVQSGAEVKKPGSSVKVSCKASGYSFTTYYIHWVRQAPGQGLEWMGWFSPGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 213 - protein, 119 aa, synthetic construct
QVQLVQSGAEVKKPGSSVKVSCKASGYSFTTYYIHWVRQAPGQGLEWMGWFSPGSGNTKYNEKFKGRVTITADTSTSTAYMELSSLRSEDTAVYYCAVSYYSYDVLDYWGQGTTVTVSS

SEQ ID NO: 214 - protein, 246 aa, synthetic construct
QVQLVQSGAEVKKPGSSVKVSCKASGYSFTTYYIHWVRQAPGQGLEWMGWFSPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 215 - protein, 119 aa, synthetic construct
QVQLVQSGAEVKKPGSSVKVSCKASGYSFTTYYIHWVRQAPGQGLEWMGWFSPGSGNTKYNEKFKGRVTITADTSTSTAYMELSSLRSEDTAVYYCAGSYYSYDVLDYWGQGTTVTVSS

SEQ ID NO: 216 - protein, 246 aa, synthetic construct
QVQLVQSGAEVKKPGSSVKVSCKASGYSFTTYYIHWVRQAPGQGLEWMGRIFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 217 - protein, 119 aa, synthetic construct
QVQLVQSGAEVKKPGSSVKVSCKASGYSFTTYYIHWVRQAPGQGLEWMGRIFPGSGNIKYNEKFKGRVTITADTSTSTAYMELSSLRSEDTAVYYCAGSYYSYDVLDYWGQGTTVTVSS

SEQ ID NO: 218 - protein, 246 aa, synthetic construct
QVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSIYSAGVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 219 - protein, 119 aa, synthetic construct
QVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSIYSAGVLDYWGQGTTVTVSS

SEQ ID NO: 220 - protein, 246 aa, synthetic construct
QVQLVQSGAEVKKPGSSVKVSCKASGYSFTLGYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 221 - protein, 119 aa, synthetic construct
QVQLVQSGAEVKKPGSSVKVSCKASGYSFTLGYIHWVRQAPGQGLEWMGWFFPGSGNIKYNEKFKGRVTITADTSTSTAYMELSSLRSEDTAVYYCAGSYYSYDVLDYWGQGTTVTVSS

SEQ ID NO: 222 - protein, 246 aa, synthetic construct
QVQLVQSGAEVKKPGSSVKVSCKASGYSFRLTYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 223 - protein, 119 aa, synthetic construct
QVQLVQSGAEVKKPGSSVKVSCKASGYSFRLTYIHWVRQAPGQGLEWMGWFFPGSGNIKYNEKFKGRVTITADTSTSTAYMELSSLRSEDTAVYYCAGSYYSYDVLDYWGQGTTVTVSS

SEQ ID NO: 224 - protein, 246 aa, synthetic construct
QVQLVQSGAEVKKPGSSVKVSCKASGYSFHNWYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 225 - protein, 119 aa, synthetic construct
QVQLVQSGAEVKKPGSSVKVSCKASGYSFHNWYIHWVRQAPGQGLEWMGWFFPGSGNIKYNEKFKGRVTITADTSTSTAYMELSSLRSEDTAVYYCAGSYYSYDVLDYWGQGTTVTVSS

SEQ ID NO: 226-229 - skipped (4 entries)

SEQ ID NO: 230 - protein, 107 aa, synthetic construct
  VARIANT 30: note = G, E, A or D
  VARIANT 31: note = N or D
  VARIANT 32: note = R or K
  VARIANT 36: note = Y or H
  VARIANT 56: note = K or S
DIQMTQSPSFLSASVGDRVTITCKASQNVXXXVAWXQQKPGKAPKALIYSSSHRYXGVPSRFSGSGSGTEFTLTISSLQPEDFATYFCQQFKSYPLTFGQGTKLEIK

SEQ ID NO: 231 - protein, 119 aa, synthetic construct
  VARIANT 27: note = H, T, G or Y
  VARIANT 28: note = D, T or S
  VARIANT 30: note = H, R, D, K or T
  VARIANT 31: note = L, D, K, T or N
  VARIANT 32: note = W, F, T, I, Y or G
  VARIANT 50: note = R or W
  VARIANT 51: note = V, I or F
  VARIANT 52: note = F, S or Y
  VARIANT 53: note = A or P
  VARIANT 57: note = N or S
  VARIANT 58: note = T, V, Y or I
  VARIANT 59: note = K or R
  VARIANT 98: note = G or V
  VARIANT 100: note = Y or I
  VARIANT 103: note = Y or A
  VARIANT 104: note = D or G
QVQLVQSGAEVKKPGSSVKVSCKASGXXFXXXYIHWVRQAPGQGLEWMGXXXXGSGXXXYNEKFKGRVTITADTSTSTAYMELSSLRSEDTAVYYCAXSXYSXXVLDYWGQGTTVTVSS

SEQ ID NO: 232 - protein, 107 aa, synthetic construct
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPRTFGGGTKVEIK

SEQ ID NO: 233 - protein, 120 aa, synthetic construct
QVQLKESGPGLVAPSQSLSITCTVSGFSLTAYGVNWVRQPPGKGLEWLGMIWGDGNTDYNSALKSRLSISKDNSKSQVFLKMNSLQTDDTARYYCARDRVTATLYAMDYWGQGTSVTVSS

SEQ ID NO: 234-1003 - skipped (770 entries)

SEQ ID NO: 1004 - protein, 721 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIKGGGGSQVTLRESGPALVKPTQTLTLTCTFSGFSLSTSGMGVGWIRQPPGKALEWLAHIWWDDDKRYNPALKSRLTISKDTSKNQVFLTMTNMDPVDTATYYCARMELWSYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1005 - protein, 233 aa, synthetic construct
METPAQLLFLLLLWLPDTTGENVLTQSPATLSLSPGERATLSCSASSSVSYMHWYQQKPGQAPRLLIYDTSKLASGIPARFSGSGSGTDHTLTISSLEPEDFAVYYCFQGSVYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1006 - protein, 721 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIKGGGGSQVQLVESGGGVVQPGRSLRLSCAASGIDFSRYWMSWVRQAPGKGLEWVGEINPDSSTINYAPSLKDRFTISRDNSKNTLYLQMSSLRAEDTAVYYCASLYYDYGDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 1007 - protein, 234 aa, synthetic construct
METDTLLLWVLLLWVPGSTGDIQMTQSPSSLSASVGDRVTITCKASQSVDSNVAWYQQKPEKAPKALIFSASLRFSGVPSRFSGSGSGTDFTLTISSLQPEDFATYFCQQYNNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1008-1099 - skipped (92 entries)

SEQ ID NO: 1100 - protein, 119 aa, synthetic construct; identical to SEQ ID NO: 9

SEQ ID NO: 1101 - protein, 106 aa, synthetic construct
QSVLTQPPSVSEAPRQRVTISCKASQNVGINVVWHQQLPGKAPKALIYSSSHRYSGVSDRFSGSGSGTSFSLAISGLQSEDEADYFCQQFKSYPLTFGTGTKVTVL

SEQ ID NO: 1102 - protein, 5 aa, synthetic construct
SAYMH

SEQ ID NO: 1103 - protein, 17 aa, synthetic construct
RIDPATGKTKYAPKFQA

SEQ ID NO: 1104 - protein, 10 aa, synthetic construct
SLNWDYGLDY

SEQ ID NO: 1105 - protein, 7 aa, synthetic construct
GFNIKSA

SEQ ID NO: 1106 - protein, 6 aa, synthetic construct
DPATGK

SEQ ID NO: 1107 - protein, 15 aa, synthetic construct
RASKSVSILGTHLIH

SEQ ID NO: 1108 - protein, 7 aa, synthetic construct
AASNLES

SEQ ID NO: 1109 - protein, 9 aa, synthetic construct
QQSIEDPWT

SEQ ID NO: 1110 - protein, 11 aa, synthetic construct
SKSVSILGTHL

SEQ ID NO: 1111 - protein, 111 aa, synthetic construct
DIVLTQSPASLAVSLGQRATISCRASKSVSILGTHLIHWYQQKPGQPPKLLIYAASNLESGVPARFSGSGSETVFTLNIHPVEEEDAATYFCQQSIEDPWTFGGGTKLGIK

SEQ ID NO: 1112 - protein, 119 aa, synthetic construct
EVQLQQSVADLVRPGASLKLSCTASGFNIKSAYMHWVIQRPDQGPECLGRIDPATGKTKYAPKFQAKATITADTSSNTAYLQLSSLTSEDTAIYYCTRSLNWDYGLDYWGQGTSVTVSS

SEQ ID NO: 1113 - protein, 119 aa, synthetic construct
QVQLVQSGAEVKKPGASVKVSCKASGFNIKSAYMHWVRQAPGQGLEWMGRIDPATGKTKYAPKFQARVTMTADTSTNTAYMELSSLRSEDTAVYYCARSLNWDYGLDYWGQGTLVTVSS

SEQ ID NO: 1114 - protein, 119 aa, synthetic construct
QVQLVQSGAEVKKPGASVKVSCKASGFNIKSAYMHWVRQAPGQEPGCMGRIDPATGKTKYAPKFQARVTMTADTSINTAYTELSSLRSEDTATYYCARSLNWDYGLDYWGQGTLVTVSS

SEQ ID NO: 1115 - protein, 119 aa, synthetic construct
QVQLVQSGAEVKKPGSSVKVSCKASGFNIKSAYMHWVRQAPGQGLEWMGRIDPATGKTKYAPKFQARVTITADTSTNTAYMELSSLRSEDTAVYYCARSLNWDYGLDYWGQGTLVTVSS

SEQ ID NO: 1116 - protein, 119 aa, synthetic construct
QVQLVQSGAEVKKPGASVKVSCKASGFNIKSAYMHWVRQAPGQRLEWMGRIDPATGKTKYAPKFQARVTITADTSANTAYMELSSLRSEDTAVYYCARSLNWDYGLDYWGQGTLVTVSS

SEQ ID NO: 1117 - protein, 109 aa, synthetic construct
EIVLTQSPATLSLSPGERATLSCRASKSVSILGTHLIHWYQQKPGQAPRLLIYAASNLESGIPARFSGSGSETDFTLTISSLEPEDFAVYFCQQSIEDPFGGGTKVEIK

SEQ ID NO: 1118 - protein, 109 aa, synthetic construct
EIVLTQSPATLSLSPGERATLSCRASKSVSILGTHLIHWYQQKPGLAPRLLIYAASNLESGIPDRFSGSGSETDFTLTISRLEPEDFAVYFCQQSIEDPFGGGTKVEIK

SEQ ID NO: 1119 - protein, 109 aa, synthetic construct
EIVLTQSPGTLSLSPGERATLSCRASKSVSILGTHLIHWYQQKPGQAPRLLIYAASNLESGIPDRFSGSGSETDFTLTISRLEPEDFAVYFCQQSIEDPFGGGTKVEIK

SEQ ID NO: 1120 - protein, 5 aa, synthetic construct
DTYMY

SEQ ID NO: 1121 - protein, 17 aa, synthetic construct
RIDPANGNTKYDPKFQD

SEQ ID NO: 1122 - protein, 9 aa, synthetic construct
GSYYYAMDY

SEQ ID NO: 1123 - protein, 7 aa, synthetic construct
GFKTEDT

SEQ ID NO: 1124 - protein, 6 aa, synthetic construct
DPANGN

SEQ ID NO: 1125 - protein, 10 aa, synthetic construct
GFKTEDTYMY

SEQ ID NO: 1126 - protein, 15 aa, synthetic construct
RASESVDSYGNSFMH

SEQ ID NO: 1127 - protein, 7 aa, synthetic construct
RASNLES

SEQ ID NO: 1128 - protein, 9 aa, synthetic construct
QQSNEDPYT

SEQ ID NO: 1129 - protein, 111 aa, synthetic construct
DIVLTQSPASLAVSLGQRATISCRASESVDSYGNSFMHWYQQKPGQPPKLLIYRASNLESGIPARFSGSGSRTDFTLTINPVEADDVATYYCQQSNEDPYTFGGGTKLEIK

SEQ ID NO: 1130 - protein, 118 aa, synthetic construct
EVQLQQSGAELVKPGASVKLSCTASGFKTEDTYMYWVKQRPEQGLEWIGRIDPANGNTKYDPKFQDKATITADSSSNTAYLQLSSLPSEDTAVYYCARGSYYYAMDYWGQGTSVTVSS

SEQ ID NO: 1131 - protein, 118 aa, synthetic construct
QVQLVQSGAEVKKPGSSVKVSCKASGFKTEDTYMYWVRQAPGQGLEWIGRIDPANGNTKYDPKFQDRATITADSSTNTAYMELSSLRSEDTAVYYCARGSYYYAMDYWGQGTLVTVSS

SEQ ID NO: 1132 - protein, 118 aa, synthetic construct
QVQLVQSGAEVKKPGASVKVSCKASGFKTEDTYMYWVRQAPGQRLEWIGRIDPANGNTKYDPKFQDRATITADSSANTAYMELSSLRSEDTAVYYCARGSYYYAMDYWGQGTLVTVSS

SEQ ID NO: 1133 - protein, 118 aa, synthetic construct
EVQLVESGGGLVQPGGSLKLSCAASGFKTEDTYMYWVRQASGKGLEWIGRIDPANGNTKYDPKFQDRATISADSSKNTAYLQMNSLKTEDTAVYYCARGSYYYAMDYWGQGTLVTVSS

SEQ ID NO: 1134 - protein, 118 aa, synthetic construct
EVQLVQSGAEVKKPGESLRISCKASGFKTEDTYMYWVRQMPGKGLEWIGRIDPANGNTKYDPKFQDQATISADSSINTAYLQWSSLKASDTAMYYCARGSYYYAMDYWGQGTLVTVSS

SEQ ID NO: 1135 - protein, 118 aa, synthetic construct
QVQLVQSGSELKKPGASVKVSCKASGFKTEDTYMYWVRQAPGQGLEWIGRIDPANGNTKYDPKFQDRAVISADSSVNTAYLQISSLKAEDTAVYYCARGSYYYAMDYWGQGTLVTVSS

SEQ ID NO: 1136 - protein, 111 aa, synthetic construct
DIVLTQSPDSLAVSLGERATINCRASESVDSYGNSFMHWYQQKPGQPPKLLIYRASNLESGVPDRFSGSGSRTDFTLTISSLQAEDVAVYYCQQSNEDPYTFGQGTKLEIK

SEQ ID NO: 1137 - protein, 111 aa, synthetic construct
EIVLTQSPATLSLSPGERATLSCRASESVDSYGNSFMHWYQQKPGQAPKLLIYRASNLESGIPARFSGSGSRTDFTLTISRLEPEDFAVYYCQQSNEDPYTFGQGTKLEIK

SEQ ID NO: 1138 - protein, 111 aa, synthetic construct
DIQLTQSPSSLSASVGDRVTITCRASESVDSYGNSFMHWYQQKPGQAPKLLIYRASNLESGVPSRFSGSGSRTDFTLTISSLQPEDVATYYCQQSNEDPYTFGQGTKLEIK

SEQ ID NO: 1139 - protein, 111 aa, synthetic construct
AIQLTQSPSSLSASVGDRVTITCRASESVDSYGNSFMHWYQQKPGKAPKLLIYRASNLESGVPSRFSGSGSRTDFTLTISSLQPEDFATYYCQQSNEDPYTFGQGTKLEIK

SEQ ID NO: 1140 - protein, 111 aa, synthetic construct
EIVLTQSPDFQSVTPKEKVTITCRASESVDSYGNSFMHWYQQKPDQSPKLLIYRASNLESGVPSRFSGSGSRTDFTLTINSLEAEDAATYYCQQSNEDPYTFGQGTKLEIK

SEQ ID NO: 1141 - protein, 5 aa, synthetic construct
SYAMS

SEQ ID NO: 1142 - protein, 17 aa, synthetic construct
HISNGGDYIYYADTVKG

SEQ ID NO: 1143 - protein, 12 aa, synthetic construct
PSYYSDPWFFDV

SEQ ID NO: 1144 - protein, 7 aa, synthetic construct
GFTFRSY

SEQ ID NO: 1145 - protein, 6 aa, synthetic construct
SNGGDY

SEQ ID NO: 1146 - protein, 10 aa, synthetic construct
GFTFRSYAMS

SEQ ID NO: 1147 - protein, 10 aa, synthetic construct
SAGSSVSFMH

SEQ ID NO: 1148 - protein, 7 aa, synthetic construct
DTSKLAS

SEQ ID NO: 1149 - protein, 9 aa, synthetic construct
LQGSGFPLT

SEQ ID NO: 1150 - protein, 6 aa, synthetic construct
GSSVSF

SEQ ID NO: 1151 - protein, 106 aa, synthetic construct
ENVLTQSPAIMSASPGEKVTMTCSAGSSVSFMHWYQQKSSTSPKLWIYDTSKLASGVPGRFSGSGSGNSFSLTISSMEAEDVAIYYCLQGSGFPLTFGSGTKLEIK

SEQ ID NO: 1152 - protein, 121 aa, synthetic construct
DVKLVESGEGLVKPGGSLKLSCAASGFTFRSYAMSWVRQTPEKRLEWVAHISNGGDYIYYADTVKGRFTISRDNARNTLYLQMSSLKSEDTAMYYCTRPSYYSDPWFFDVWGTGTTVTVSS

SEQ ID NO: 1153 - protein, 121 aa, synthetic construct
EVQLVESGGGLVQPGGSLRLSCAASGFTFRSYAMSWVRQAPGKGLEWVAHISNGGDYIYYADTVKGRFTISRDNAKNSLYLQMNSLRAEDTAVYYCTRPSYYSDPWFFDVWGQGTTVTVSS

SEQ ID NO: 1154 - protein, 121 aa, synthetic construct
QVQLVESGGGVVQPGRSLRLSCAASGFTFRSYAMSWVRQAPGKGLEWVAHISNGGDYIYYADTVKGRFTISRDNSKNTLYLQMSSLRAEDTAVYYCTRPSYYSDPWFFDVWGQGTTVTVSS

SEQ ID NO: 1155 - protein, 121 aa, synthetic construct
EVQLVESGGGLVQPGGSLRLSCAASGFTFRSYAMSWVRQAPGKGLEWVAHISNGGDYIYYADTVKGRFTISRDNSKNTLYLQMNSLRAEDTAVYYCTRPSYYSDPWFFDVWGQGTTVTVSS

SEQ ID NO: 1156 - protein, 121 aa, synthetic construct
QVQLVQSGSELKKPGASVKVSCKASGFTFRSYAMSWVRQAPGQGLEWVAHISNGGDYIYYADTVKGRFVISRDNSVNTLYLQISSLKAEDTAVYYCTRPSYYSDPWFFDVWGQGTTVTVSS

SEQ ID NO: 1157 - protein, 121 aa, synthetic construct
QVQLVQSGAEVKKPGASVKVSCKASGFTFRSYAMSWVRQAPGQRLEWVAHISNGGDYIYYADTVKGRFTITRDNSANTLYMELSSLRSEDTAVYYCTRPSYYSDPWFFDVWGQGTTVTVSS

SEQ ID NO: 1158 - protein, 106 aa, synthetic construct
ENVLTQSPATLSLSPGERATLSCSAGSSVSFMHWYQQKPGQAPKLLIYDTSKLASGIPARFSGSGSGNDFTLTISSLEPEDFAVYYCLQGSGFPLTFGQGTKLEIK

SEQ ID NO: 1159 - protein, 106 aa, synthetic construct
ENVLTQSPDFQSVTPKEKVTITCSAGSSVSFMHWYQQKPDQSPKLLIYDTSKLASGVPSRFSGSGSGNDFTLTINSLEAEDAATYYCLQGSGFPLTFGQGTKLEIK

SEQ ID NO: 1160 - protein, 106 aa, synthetic construct
DNQLTQSPSSLSASVGDRVTITCSAGSSVSFMHWYQQKPGKVPKLLIYDTSKLASGVPSRFSGSGSGNDFTLTISSLQPEDVATYYCLQGSGFPLTFGQGTKLEIK

SEQ ID NO: 1161 - protein, 106 aa, synthetic construct
ANQLTQSPSSLSASVGDRVTITCSAGSSVSFMHWYQQKPGKAPKLLIYDTSKLASGVPSRFSGSGSGNDFTLTISSLQPEDFATYYCLQGSGFPLTFGQGTKLEIK

SEQ ID NO: 1162 - protein, 106 aa, synthetic construct
DNVLTQSPDSLAVSLGERATINCSAGSSVSFMHWYQQKPGQPPKLLIYDTSKLASGVPDRFSGSGSGNDFTLTISSLQAEDVAVYYCLQGSGFPLTFGQGTKLEIK

SEQ ID NO: 1163 - protein, 5 aa, synthetic construct
DYNIH

SEQ ID NO: 1164 - protein, 17 aa, synthetic construct
YINPYNGRTGYNQKFKA

SEQ ID NO: 1165 - protein, 9 aa, synthetic construct
WDGSSYFDY

SEQ ID NO: 1166 - protein, 10 aa, synthetic construct
GYTFTDYNIH

SEQ ID NO: 1167 - protein, 6 aa, synthetic construct
NPYNGR

SEQ ID NO: 1168 - protein, 10 aa, synthetic construct
SASSSVSYMH

SEQ ID NO: 1169 - protein, 7 aa, synthetic construct
EISKLAS

SEQ ID NO: 1170 - protein, 9 aa, synthetic construct
QQWNYPLLT

SEQ ID NO: 1171 - protein, 106 aa, synthetic construct
EIVLTQSPAITAASLGQKVTITCSASSSVSYMHWYQQKSGTSPKPWIYEISKLASGVPARFSGSGSGTSYSLTISSMEAEDAAIYYCQQWNYPLLTFGAGTKLELK

SEQ ID NO: 1172 - protein, 118 aa, synthetic construct
EVQLQQSGPVLVKPGASVRMSCKASGYTFTDYNIHWVKQSHGRSLEWVGYINPYNGRTGYNQKFKAKATLTVDKSSSTAYMDLRSLTSEDSAVYYCARWDGSSYFDYWGQGTTLTVSS

SEQ ID NO: 1173 - protein, 118 aa, synthetic construct
QVQLVQSGAEVKKPGSSVKVSCKASGYTFTDYNIHWVRQAPGQGLEWVGYINPYNGRTGYNQKFKARATLTVDKSTSTAYMELSSLRSEDTAVYYCARWDGSSYFDYWGQGTTVTVSS

SEQ ID NO: 1174 - protein, 118 aa, synthetic construct
QVQLVQSGAEVKKPGASVKVSCKASGYTFTDYNIHWVRQAPGQGLEWVGYINPYNGRTGYNQKFKARATLTVDKSTSTAYMELRSLRSDDMAVYYCARWDGSSYFDYWGQGTTVTVSS

SEQ ID NO: 1175 - protein, 118 aa, synthetic construct
QVQLVQSGAEVKKPGASVKVSCKASGYTFTDYNIHWVRQATGQGLEWVGYINPYNGRTGYNQKFKARATLTVNKSISTAYMELSSLRSEDTAVYYCARWDGSSYFDYWGQGTTVTVSS

SEQ ID NO: 1176 - protein, 118 aa, synthetic construct
EVQLVESGGGLVQPGRSLRLSCTASGYTFTDYNIHWVRQAPGKGLEWVGYINPYNGRTGYNQKFKARATLSVDKSKSIAYLQMNSLKTEDTAVYYCARWDGSSYFDYWGQGTTVTVSS

SEQ ID NO: 1177 - protein, 118 aa, synthetic construct
QVQLVQSGSELKKPGASVKVSCKASGYTFTDYNIHWVRQAPGQGLEWVGYINPYNGRTGYNQKFKARAVLSVDKSVSTAYLQISSLKAEDTAVYYCARWDGSSYFDYWGQGTTVTVSS

SEQ ID NO: 1178 - protein, 106 aa, synthetic construct
EIVLTQSPATLSLSPGERATLSCSASSSVSYMHWYQQKPGQAPKLLIYEISKLASGIPARFSGSGSGTDYTLTISSLEPEDFAVYYCQQWNYPLLTFGQGTKLEIK

SEQ ID NO: 1179 - protein, 106 aa, synthetic construct
EIVLTQSPATLSLSPGERATLSCSASSSVSYMHWYQQKPGQAPKLLIYEISKLASGIPARFSGSGSGTDYTLTISRLEPEDFAVYYCQQWNYPLLTFGQGTKLEIK

SEQ ID NO: 1180 - protein, 106 aa, synthetic construct
EIVLTQSPDFQSVTPKEKVTITCSASSSVSYMHWYQQKPDQSPKLLIYEISKLASGVPSRFSGSGSGTDYTLTINSLEAEDAATYYCQQWNYPLLTFGQGTKLEIK

SEQ ID NO: 1181 - protein, 106 aa, synthetic construct
DIQLTQSPSFLSASVGDRVTITCSASSSVSYMHWYQQKPGKAPKLLIYEISKLASGVPSRFSGSGSGTEYTLTISSLQPEDFATYYCQQWNYPLLTFGQGTKLEIK

SEQ ID NO: 1182 - protein, 106 aa, synthetic construct
AIQLTQSPSSLSASVGDRVTITCSASSSVSYMHWYQQKPGKAPKLLIYEISKLASGVPSRFSGSGSGTDYTLTISSLQPEDFATYYCQQWNYPLLTFGQGTKLEIK

SEQ ID NO: 1183 - protein, 106 aa, synthetic construct
AIRLTQSPFSLSASVGDRVTITCSASSSVSYMHWYQQKPAKAPKLFIYEISKLASGVPSRFSGSGSGTDYTLTISSLQPEDFATYYCQQWNYPLLTFGQGTKLEIK

SEQ ID NO: 1184 - protein, 106 aa, synthetic construct
DIVLTQSPDSLAVSLGERATINCSASSSVSYMHWYQQKPGQPPKLLIYEISKLASGVPDRFSGSGSGTDYTLTISSLQAEDVAVYYCQQWNYPLLTFGQGTKLEIK

SEQ ID NO: 1185 - protein, 5 aa, synthetic construct
GSWMN

SEQ ID NO: 1186 - protein, 17 aa, synthetic construct
RIYPGDGDTDYSGKFKG

SEQ ID NO: 1187 - protein, 12 aa, synthetic construct
SGYFNYVPVFDY

SEQ ID NO: 1188 - protein, 7 aa, synthetic construct
GYTFSGS

SEQ ID NO: 1189 - protein, 6 aa, synthetic construct
YPGDGD

SEQ ID NO: 1190 - protein, 10 aa, synthetic construct
GYTFSGSWMN

SEQ ID NO: 1191 - protein, 10 aa, synthetic construct
SANSTVGYIH

SEQ ID NO: 1192 - protein, 7 aa, synthetic construct
TTSNLAS

SEQ ID NO: 1193 - protein, 8 aa, synthetic construct
HQWSFYPT

SEQ ID NO: 1194 - protein, 6 aa, synthetic construct
NSTVGY

SEQ ID NO: 1195 - protein, 105 aa, synthetic construct
QIVLTQSPAIMSASLGEEIALTCSANSTVGYIHWYQQKSGTSPKLLIYTTSNLASGVPSRFSGSGSGTFYSLTISSVEAEDAADYFCHQWSFYPTFGGGTKLEIK

SEQ ID NO: 1196 - protein, 121 aa, synthetic construct
QIQLQQSGPEVVKPGASVQISCKASGYTFSGSWMNWVKQRPGKGLEWIGRIYPGDGDTDYSGKFKGRATLTADKSSSTAYMRLSSLTSEDSAVYFCARSGYFNYVPVFDYWGQGTTLSVSS

SEQ ID NO: 1197 - protein, 121 aa, synthetic construct
QIQLVQSGAEVKKPGASVKVSCKASGYTFSGSWMNWVRQAPGQGLEWIGRIYPGDGDTDYSGKFKGRATLTADKSTSTAYMELSSLRSEDTAVYYCARSGYFNYVPVFDYWGQGTTVTVSS

SEQ ID NO: 1198 - protein, 121 aa, synthetic construct
QIQLVQSGAEVKKPGSSVKVSCKASGYTFSGSWMNWVRQAPGQGLEWIGRIYPGDGDTDYSGKFKGRATLTADKSTSTAYMELSSLRSEDTAVYYCARSGYFNYVPVFDYWGQGTTVTVSS

SEQ ID NO: 1199 - protein, 121 aa, synthetic construct
EIQLVQSGAEVKKPGESLKISCKASGYTFSGSWMNWVRQMPGKGLEWIGRIYPGDGDTDYSGKFKGQATLSADKSISTAYLQWSSLKASDTAMYYCARSGYFNYVPVFDYWGQGTTVTVSS

SEQ ID NO: 1200 - protein, 121 aa, synthetic construct
QIQLVQSGSELKKPGASVKVSCKASGYTFSGSWMNWVRQAPGQGLEWIGRIYPGDGDTDYSGKFKGRAVLSADKSVSTAYLQISSLKAEDTAVYYCARSGYFNYVPVFDYWGQGTTVTVSS

SEQ ID NO: 1201 - protein, 121 aa, synthetic construct
QIQLVQSGSELKKPGASVKVSCKASGYTFSGSWMNWVRQAPGQGLEWIGRIYPGDGDTDYSGKFKGRAVLSADKSVSMAYLQISSLKAEDTAVYYCARSGYFNYVPVFDYWGQGTTVTVSS

SEQ ID NO: 1202 - protein, 121 aa, synthetic construct
EIQLVESGGGLVQPGRSLRLSCTASGYTFSGSWMNWVRQAPGKGLEWIGRIYPGDGDTDYSGKFKGRATLSADKSKSIAYLQMNSLKTEDTAVYYCARSGYFNYVPVFDYWGQGTTVTVSS

SEQ ID NO: 1203 - protein, 105 aa, synthetic construct
EIVLTQSPATLSLSPGERATLSCSANSTVGYIHWYQQKPGQAPKLLIYTTSNLASGIPARFSGSGSGTDYTLTISSLEPEDFAVYFCHQWSFYPTFGQGTKLEIK

SEQ ID NO: 1204 - protein, 105 aa, synthetic construct
DIQLTQSPSFLSASVGDRVTITCSANSTVGYIHWYQQKPGKAPKLLIYTTSNLASGVPSRFSGSGSGTEYTLTISSLQPEDFATYFCHQWSFYPTFGQGTKLEIK

SEQ ID NO: 1205 - protein, 105 aa, synthetic construct
EIVLTQSPATLSLSPGERATLSCSANSTVGYIHWYQQKPGQAPKLLIYTTSNLASGIPARFSGSGPGTDYTLTISSLEPEDFAVYFCHQWSFYPTFGQGTKLEIK

SEQ ID NO: 1206 - protein, 105 aa, synthetic construct
DIVLTQSPDSLAVSLGERATINCSANSTVGYIHWYQQKPGQPPKLLIYTTSNLASGVPDRFSGSGSGTDYTLTISSLQAEDVAVYFCHQWSFYPTFGQGTKLEIK

SEQ ID NO: 1207 - protein, 105 aa, synthetic construct
EIVLTQSPDFQSVTPKEKVTITCSANSTVGYIHWYQQKPDQSPKLLIYTTSNLASGVPSRFSGSGSGTDYTLTINSLEAEDAATYFCHQWSFYPTFGQGTKLEIK

SEQ ID NO: 1208 - protein, 5 aa, synthetic construct
DYYMY

SEQ ID NO: 1209 - protein, 17 aa, synthetic construct
TISGGGSYTYSPDSVKG

SEQ ID NO: 1210 - protein, 14 aa, synthetic construct
ERDIYYGNFNAMVY

SEQ ID NO: 1211 - protein, 7 aa, synthetic construct
GFTFSDY

SEQ ID NO: 1212 - protein, 6 aa, synthetic construct
SGGGSY

SEQ ID NO: 1213 - protein, 10 aa, synthetic construct
GFTFSDYYMY

SEQ ID NO: 1214 - protein, 15 aa, synthetic construct
RASKSVSTSGYSYMH

SEQ ID NO: 1215 - protein, 7 aa, synthetic construct
LASNLES

SEQ ID NO: 1216 - protein, 9 aa, synthetic construct
QHSRDLPWT

SEQ ID NO: 1217 - protein, 11 aa, synthetic construct
SKSVSTSGYSY

SEQ ID NO: 1218 - protein, 111 aa, synthetic construct
DIVLTQSPVSLTVSLGQRATISCRASKSVSTSGYSYMHWYQQKPGQPPKLLIYLASNLESGVPARFSGSGSGTDFTLNIHPVEEEDAATYYCQHSRDLPWTFGGGTKLEIK

SEQ ID NO: 1219 - protein, 123 aa, synthetic construct
EVQLVESGGGLVKPGGSLKLSCAASGFTFSDYYMYWVRQTPEKRLEWVATISGGGSYTYSPDSVKGRFTISRDNAKNNLYLQMSSLRSEDTAMYFCARERDIYYGNFNAMVYWGRGTSVTVSS

SEQ ID NO: 1220 - protein, 123 aa, synthetic construct
EVQLLESGGGLVQPGGSLRLSCAASGFTFSDYYMYWVRQAPGKGLEWVATISGGGSYTYSPDSVKGRFTISRDNSKNTLYLQMNSLRAEDTAVYYCARERDIYYGNFNAMVYWGRGTLVTVSS

SEQ ID NO: 1221 - protein, 123 aa, synthetic construct
EVQLVESGGGLVQPGGSLRLSCAASGFTFSDYYMYWVRQAPGKGLEWVATISGGGSYTYSPDSVKGRFTISRDNAKNSLYLQMNSLRAEDTAVYYCARERDIYYGNFNAMVYWGRGTLVTVSS

SEQ ID NO: 1222 - protein, 123 aa, synthetic construct
QVQLVESGGGVVQPGRSLRLSCAASGFTFSDYYMYWVRQAPGKGLEWVATISGGGSYTYSPDSVKGRFTISRDNSKNTLYLQMNSLRAEDTAVYYCARERDIYYGNFNAMVYWGRGTLVTVSS

SEQ ID NO: 1223 - protein, 123 aa, synthetic construct
QVQLVESGGGLVKPGGSLRLSCAASGFTFSDYYMYWIRQAPGKGLEWVATISGGGSYTYSPDSVKGRFTISRDNAKNSLYLQMNSLRAEDTAVYYCARERDIYYGNFNAMVYWGRGTLVTVSS

SEQ ID NO: 1224 - protein, 111 aa, synthetic construct
EIVLTQSPGTLSLSPGERATLSCRASKSVSTSGYSYMHWYQQKPGQAPRLLIYLASNLESGIPDRFSGSGSGTDFTLTISRLEPEDFAVYYCQHSRDLPWTFGGGTKVEIK

SEQ ID NO: 1225 - protein, 111 aa, synthetic construct
EIVLTQSPATLSLSPGERATLSCRASKSVSTSGYSYMHWYQQKPGQAPRLLIYLASNLESGIPARFSGSGSGTDFTLTISSLEPEDFAVYYCQHSRDLPWTFGGGTKVEIK

SEQ ID NO: 1226 - protein, 111 aa, synthetic construct
DIQLTQSPSTLSASVGDRVTITCRASKSVSTSGYSYMHWYQQKPGKAPKLLIYLASNLESGVPSRFSGSGSGTEFTLTISSLQPDDFATYYCQHSRDLPWTFGGGTKVEIK

SEQ ID NO: 1227 - protein, 111 aa, synthetic construct
AIQLTQSPSSLSASVGDRVTITCRASKSVSTSGYSYMHWYQQKPGKAPKLLIYLASNLESGVPSRFSGSGSGTDFTLTISSLQPEDFATYYCQHSRDLPWTFGGGTKVEIK

SEQ ID NO: 1228 - skipped

SEQ ID NO: 1229 - protein, 5 aa, synthetic construct
GYFWN

SEQ ID NO: 1230 - protein, 16 aa, synthetic construct
YISYDGSNNYNPSLKN

SEQ ID NO: 1231 - protein, 11 aa, synthetic construct
PSPGTGYAVDY

SEQ ID NO: 1232 - protein, 8 aa, synthetic construct
GYSITSGY

SEQ ID NO: 1233 - protein, 6 aa, synthetic construct
SYDGSN

SEQ ID NO: 1234 - protein, 11 aa, synthetic construct
GYSITSGYFWN

SEQ ID NO: 1235 - protein, 16 aa, synthetic construct
RSSQSLVHSNGNTYLH

SEQ ID NO: 1236 - protein, 7 aa, synthetic construct
KVSNRFS

SEQ ID NO: 1237 - protein, 9 aa, synthetic construct
SQSTHVPFT

SEQ ID NO: 1238 - protein, 12 aa, synthetic construct
SQSLVHSNGNTY

SEQ ID NO: 1239 - protein, 112 aa, synthetic construct
NVVMTQTPLSLPVSLGDQASISCRSSQSLVHSNGNTYLHWYLQKPGQSPKFLIYKVSNRFSGVPDRFSGGGSGTEFTLKISRVEAEDLGVYFCSQSTHVPFTFGSGTKLEIK

SEQ ID NO: 1240 - protein, 120 aa, synthetic construct
NVQLQESGPGLVKPSQSLSLTCSVAGYSITSGYFWNWIRQFPGNKLEWMGYISYDGSNNYNPSLKNRISITRDTSKNQFFLKLNSVTTEDTATYYCASPSPGTGYAVDYWGQGTSVTVSS

SEQ ID NO: 1241 - protein, 120 aa, synthetic construct
QVQLQESGPGLVKPSETLSLTCTVSGYSITSGYFWNWIRQPPGKGLEWIGYISYDGSNNYNPSLKNRVTISRDTSKNQFSLKLSSVTAADTAVYYCASPSPGTGYAVDYWGQGTLVTVSS

SEQ ID NO: 1242 - protein, 120 aa, synthetic construct; identical to SEQ ID NO: 1241

SEQ ID NO: 1243 - protein, 120 aa, synthetic construct
QVQLVESGGGLVQPGGSLRLSCSVSGYSITSGYFWNWVRQAPGKGLEWVGYISYDGSNNYNPSLKNRFTISRDTSKNTFYLQMNSLRAEDTAVYYCASPSPGTGYAVDYWGQGTLVTVSS

SEQ ID NO: 1244 - protein, 111 aa, synthetic construct
VVMTQSPGTLSLSPGERATLSCRSSQSLVHSNGNTYLHWYQQKPGQAPRFLIYKVSNRFSGIPDRFSGSGSGTDFTLTISRLEPEDFAVYFCSQSTHVPFTFGQGTKLEIK

SEQ ID NO: 1245 - protein, 112 aa, synthetic construct
EVVMTQSPATLSLSPGERATLSCRSSQSLVHSNGNTYLHWYQQKPGQAPRFLIYKVSNRFSGIPARFSGSGSGTDFTLTISSLEPEDFAVYFCSQSTHVPFTFGQGTKLEIK

SEQ ID NO: 1246 - protein, 112 aa, synthetic construct
EVVMTQSPATLSVSPGERATLSCRSSQSLVHSNGNTYLHWYQQKPGQAPRFLIYKVSNRFSGIPARFSGSGSGTEFTLTISSLQSEDFAVYFCSQSTHVPFTFGQGTKLEIK

SEQ ID NO: 1247 - protein, 112 aa, synthetic construct
DVQMTQSPSSLSASVGDRVTITCRSSQSLVHSNGNTYLHWYQQKPGKAPKFLIYKVSNRFSGVPSRFSGSGSGTDFTFTISSLQPEDIATYFCSQSTHVPFTFGQGTKLEIK

SEQ ID NO: 1248 - protein, 5 aa, synthetic construct
DYIVH

SEQ ID NO: 1249 - protein, 17 aa, synthetic construct
WINTYTGTPTYADDFEG

SEQ ID NO: 1250 - protein, 13 aa, synthetic construct
SWRRGIRGIGFDY

SEQ ID NO: 1251 - protein, 7 aa, synthetic construct
GYTFTDY

SEQ ID NO: 1252 - protein, 6 aa, synthetic construct
NTYTGT

SEQ ID NO: 1253 - protein, 10 aa, synthetic construct
GYTFTDYIVH

SEQ ID NO: 1254 - protein, 11 aa, synthetic construct
KASKSINKYLA

SEQ ID NO: 1255 - protein, 7 aa, synthetic construct
DGSTLQS

SEQ ID NO: 1256 - protein, 9 aa, synthetic construct
QQHNEYPPT

SEQ ID NO: 1257 - protein, 7 aa, synthetic construct
SKSINKY

SEQ ID NO: 1258 - protein, 107 aa, synthetic construct
DVQMTQSPYNLAASPGESVSINCKASKSINKYLAWYQQKPGKPNKLLIYDGSTLQSGIPSRFSGSGSGTDFTLTIRGLEPEDFGLYYCQQHNEYPPTFGAGTKLELK

SEQ ID NO: 1259 - protein, 122 aa, synthetic construct
QLQLVQSGPELREPGESVKISCKASGYTFTDYIVHWVKQAPGKGLKWMGWINTYTGTPTYADDFEGRFVFSLEASASTANLQISNLKNEDTATYFCARSWRRGIRGIGFDYWGQGVMVTVSS

SEQ ID NO: 1260 - protein, 122 aa, synthetic construct
QLQLVQSGAEVKKPGASVKVSCKASGYTFTDYIVHWVRQAPGQGLEWMGWINTYTGTPTYADDFEGWVTMTLDASISTAYMELSRLRSDDTAVYYCARSWRRGIRGIGFDYWGQGTMVTVSS

SEQ ID NO: 1261 - protein, 122 aa, synthetic construct
QLQLVQSGAEVKKPGASVKVSCKASGYTFTDYIVHWVRQAPGQGLEWMGWINTYTGTPTYADDFEGRVTMTLDASTSTAYMELSSLRSEDTAVYYCARSWRRGIRGIGFDYWGQGTMVTVSS

SEQ ID NO: 1262 - protein, 122 aa, synthetic construct
QLQLVQSGAEVKKPGASVKVSCKASGYTFTDYIVHWVRQAPGQRLEWMGWINTYTGTPTYADDFEGRVTITLDASASTAYMELSSLRSEDMAVYYCARSWRRGIRGIGFDYWGQGTMVTVSS

SEQ ID NO: 1263 - protein, 122 aa, synthetic construct
QLQLVQSGAEVKKPGASVKVSCKASGYTFTDYIVHWVRQATGQGLEWMGWINTYTGTPTYADDFEGRVTMTLNASISTAYMELSSLRSEDTAVYYCARSWRRGIRGIGFDYWGQGTMVTVSS

SEQ ID NO: 1264 - protein, 107 aa, synthetic construct
EVVMTQSPGTLSLSPGERATLSCKASKSINKYLAWYQQKPGQAPRLLIYDGSTLQSGIPDRFSGSGSGTDFTLTISRLEPEDFAVYYCQQHNEYPPTFGQGTKLEIK

SEQ ID NO: 1265 - protein, 107 aa, synthetic construct
EVVMTQSPATLSLSPGERATLSCKASKSINKYLAWYQQKPGQAPRLLIYDGSTLQSGIPARFSGSGSGTDFTLTISSLEPEDFAVYYCQQHNEYPPTFGQGTKLEIK

SEQ ID NO: 1266 - protein, 107 aa, synthetic construct
DVQMTQSPSSLSASVGDRVTITCKASKSINKYLAWYQQKPGKAPKLLIYDGSTLQSGVPSRFSGSGSGTDFTLTISSLQPEDFATYYCQQHNEYPPTFGQGTKLEIK

SEQ ID NO: 1267 - protein, 107 aa, synthetic construct
AVRMTQSPSSFSASTGDRVTITCKASKSINKYLAWYQQKPGKAPKLLIYDGSTLQSGVPSRFSGSGSGTDFTLTISCLQSEDFATYYCQQHNEYPPTFGQGTKLEIK

SEQ ID NO: 1268 - protein, 5 aa, synthetic construct
NYGVH

SEQ ID NO: 1269 - protein, 16 aa, synthetic construct
VIWSDGSTDYDTAFIS

SEQ ID NO: 1270 - protein, 9 aa, synthetic construct
RAVVADFDY

SEQ ID NO: 1271 - protein, 6 aa, synthetic construct
GFSLTN

SEQ ID NO: 1272 - protein, 9 aa, synthetic construct
VIWSDGSTD

SEQ ID NO: 1273 - protein, 10 aa, synthetic construct
GFSLTNYGVH

SEQ ID NO: 1274 - protein, 117 aa, synthetic construct
QVQLKQSGPGLLQPSQSLSITCTVSGFSLTNYGVHWVRQSPGKGLEWLGVIWSDGSTDYDTAFISRLSISKDNSKSQVFFKLNSLQADDTAIYYCARRAVVADFDYWGQGTTLTVSS

SEQ ID NO: 1275 - protein, 16 aa, synthetic construct
KASKEVTIFGSISALH

SEQ ID NO: 1276 - protein, 7 aa, synthetic construct
NGAKLES

SEQ ID NO: 1277 - protein, 9 aa, synthetic construct
LQNKEVPFT

SEQ ID NO: 1278 - protein, 119 aa, synthetic construct
DIVLTQSPASLAVSLGQKATISCKASKEVTIFGSISALHWYQQKPGQPPKLIYNGAKLESGVSARFSDSGSQNRSPFGNQLSFTLTIAPVEADDAATYYCLQNKEVPFTFGSGTKLEIK

SEQ ID NO: 1279 - protein, 117 aa, synthetic construct
DIVLTQSPDSLAVSLGERATINCKASKEVTIFGSISALHWYQQKPGQPPKLLYNGAKLESGVSARFGVPDRFSRSGSGLDFTLTISSLQAEDVAVYYCLQNKEVPFTFGQGTKLEIK

SEQ ID NO: 1280 - protein, 117 aa, synthetic construct
EIVLTQSPDFQSVTPKEKVTITCKASKEVTIFGSISALHWYQQKPDQSPKLLYNGAKLESGVSARFGVPSRFSRSGSGLDFTLTINSLEAEDAATYYCLQNKEVPFTFGQGTKLEIK

SEQ ID NO: 1281 - protein, 117 aa, synthetic construct
AIQLTQSPSSLSASVGDRVTITCKASKEVTIFGSISALHWYQQKPGKAPKLLYNGAKLESGVSARFGVPSRFSRSGSGLDFTLTISSLQPEDFATYYCLQNKEVPFTFGQGTKLEIK

SEQ ID NO: 1282 - protein, 117 aa, synthetic construct
DIVLTQTPLSLSVTPGQPASISCKASKEVTIFGSISALHWYLQKPGQPPKLLYNGAKLESGVSARFGVPDRFSRSGSGLDFTLKISRVEAEDVGVYYCLQNKEVPFTFGQGTKLEIK

SEQ ID NO: 1283 - protein, 117 aa, synthetic construct
QVTLKESGPVLVKPTETLTLTCTVSGFSLTNYGVHWVRQPPGKALEWLGVIWSDGSTDYDTAFISRLTISKDNSKSQVVLTMTNMDPVDTATYYCARRAVVADFDYWGQGTTVTVSS

SEQ ID NO: 1284 - protein, 117 aa, synthetic construct
QVQLQESGPGLVKPSGTLSLTCAVSGFSLTNYGVHWVRQPPGKGLEWLGVIWSDGSTDYDTAFISRLTISKDNSKSQVSLKLSSVTAADTAVYYCARRAVVADFDYWGQGTTVTVSS

SEQ ID NO: 1285 - protein, 117 aa, synthetic construct
QVQLQQSGPGLVKPSQTLSLTCAVSGFSLTNYGVHWVRQSPSRGLEWLGVIWSDGSTDYDTAFISRLTINKDNSKSQVSLQLNSVTPEDTAVYYCARRAVVADFDYWGQGTTVTVSS

SEQ ID NO: 1286 - protein, 117 aa, synthetic construct
EVQLVESGGGLVQPGPSLRLSCTVSGFSLTNYGVHWVRQAPGKGLEWLGVIWSDGSTDYDTAFISRLTISKDNSKSIVYLQMNSLKTEDTAVYYCARRAVVADFDYWGQGTTVTVSS

SEQ ID NO: 1287 - protein, 117 aa, synthetic construct
EVQLVQSGAEVKKPGESLRISCKVSGFSLTNYGVHWVRQMPGKGLEWLGVIWSDGSTDYDTAFISQLTISKDNSISTVYLQWSSLKASDTAMYYCARRAVVADFDYWGQGTTVTVSS

SEQ ID NO: 1288 - protein, 5 aa, synthetic construct
SYGMS

SEQ ID NO: 1289 - protein, 17 aa, synthetic construct
LISSGGSYTYYTDSVKG

SEQ ID NO: 1290 - protein, 8 aa, synthetic construct
HGGNFFDY

SEQ ID NO: 1291 - protein, 7 aa, synthetic construct
GFTFRSY

SEQ ID NO: 1292 - protein, 6 aa, synthetic construct
SSGGSY

SEQ ID NO: 1293 - protein, 10 aa, synthetic construct
GFTFRSYGMS

SEQ ID NO: 1294 - protein, 10 aa, synthetic construct
SVSSSVSYMH

SEQ ID NO: 1295 - protein, 7 aa, synthetic construct
DTSKLAS

SEQ ID NO: 1296 - protein, 10 aa, synthetic construct
QQWSSNPQYT

SEQ ID NO: 1297 - protein, 6 aa, synthetic construct
SSSVSY

SEQ ID NO: 1298 - protein, 5 aa, synthetic construct
SSWMN

SEQ ID NO: 1299 - protein, 17 aa, synthetic construct
RIYPGDGDTKYNGKFKG

SEQ ID NO: 1300 - protein, 10 aa, synthetic construct
RGTGGWYFDV

SEQ ID NO: 1301 - protein, 10 aa, synthetic construct
RGTGGWYFDV

SEQ ID NO: 1302 - protein, 7 aa, synthetic construct
GYAFSSS

SEQ ID NO: 1303 - protein, 6 aa, synthetic construct
YPGDGD

SEQ ID NO: 1304 - protein, 10 aa, synthetic construct
GYAFSSSWMN

SEQ ID NO: 1305 - protein, 15 aa, synthetic construct
RASESVDSSGNSFMH

SEQ ID NO: 1306 - protein, 7 aa, synthetic construct
RASNLES

SEQ ID NO: 1307 - protein, 9 aa, synthetic construct
QQSFDDPFT

SEQ ID NO: 1308 - protein, 11 aa, synthetic construct
SESVDSSGNSF

SEQ ID NO: 1309 - protein, 246 aa, synthetic construct
QVQLVQSGAEVKKPGSSVKVSCKASGHDFDKFYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 1310 - protein, 119 aa, synthetic construct; identical to SEQ ID NO: 94

SEQ ID NO: 1311 - protein, 119 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAVSYYSYDVLDYWGQGTTVTVSS

SEQ ID NO: 1312 - protein, 119 aa, synthetic construct
QVQLVQSGAEVKKPGSSVKVSCKASGHDFRLTYIHWVRQAPGQGLEWMGRISAGSGNVKYNEKFKGRVTITADTSTSTAYMELSSLRSEDTAVYYCAVSYYSYDVLDYWGQGTTVTVSS

SEQ ID NO: 1313 - protein, 107 aa, synthetic construct; identical to SEQ ID NO: 90

SEQ ID NO: 1314 - protein, 107 aa, synthetic construct; identical to SEQ ID NO: 141

SEQ ID NO: 1315 - protein, 5 aa, synthetic construct
AYGVN

SEQ ID NO: 1316 - protein, 16 aa, synthetic construct
MIWGDGNTDYNSALKS

SEQ ID NO: 1317 - protein, 12 aa, synthetic construct
DRVTATLYAMDY

SEQ ID NO: 1318 - protein, 7 aa, synthetic construct
GFSLTAY

SEQ ID NO: 1319 - protein, 5 aa, synthetic construct
WGDGN

SEQ ID NO: 1320 - protein, 10 aa, synthetic construct
GFSLTAYGVN

SEQ ID NO: 1321 - protein, 11 aa, synthetic construct
SASQGISNYLN

SEQ ID NO: 1322 - protein, 7 aa, synthetic construct
YTSSLHS

SEQ ID NO: 1323 - protein, 9 aa, synthetic construct
QQYSKLPRT

SEQ ID NO: 1324 - protein, 107 aa, synthetic construct
DIQMTQSPSSLSASVGDRVTITCSASQGISNYLNWYQQTPGKAPKLLIYYTSSLHSGVPSRFSGSGSGTDYTFTISSLQPEDIATYYCQQYSKLPRTFGQGTKLQIT

SEQ ID NO: 1325 - protein, 120 aa, synthetic construct
QVQLQESGPGLVRPSQTLSLTCTVSGFSLTAYGVNWVRQPPGRGLEWLGMIWGDGNTDYNSALKSRVTMLKDTSKNQFSLRLSSVTAADTAVYYCARDRVTATLYAMDYWGQGSLVTVSS

SEQ ID NO: 1326 - protein, 246 aa, synthetic construct
QVQLVQSGAEVKKPGSSVKVSCKASGHDFRLTYIHWVRQAPGQGLEWMGRVSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGDRVVWHQQKPGKAPKALIYSSSHRYKGVPSRFSGSGSGTEFTLTISSLQPEDFATYFCQQFKSYPLTFGQGTKLEIK

SEQ ID NO: 1327 - protein, 246 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGNRVVWHQQKPGKAPKALIYSSSHRYKGVPSRFSGSGSGTEFTLTISSLQPEDFATYFCQQFKSYPLTFGQGTKLEIK

SEQ ID NO: 1328 - protein, 246 aa, synthetic construct
QVQLVQSGAEVKKPGSSVKVSCKASGHDFRLTYIHWVRQAPGQGLEWMGRIYAGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIK

SEQ ID NO: 1329 - protein, 246 aa, synthetic construct
QVQLVQSGAEVKKPGSSVKVSCKASGHDFKLTYIHWVRQAPGQGLEWMGRVSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGDRVAWHQQKPGKAPKALIYSSSHRYKGVPSRFSGSGSGTEFTLTISSLQPEDFATYFCQQFKSYPLTFGQGTKLEIK

SEQ ID NO: 1330 - protein, 246 aa, synthetic construct
QVQLVQSGAEVKKPGSSVKVSCKASGHDFKLTYIHWVRQAPGQGLEWMGRVSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVDNRVAWHQQKPGKAPKALIYSSSHRYKGVPSRFSGSGSGTEFTLTISSLQPEDFATYFCQQFKSYPLTFGQGTKLEIK

SEQ ID NO: 1331 - protein, 246 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIK

SEQ ID NO: 1332 - protein, 246 aa, synthetic construct
QVQLVQSGAEVKKPGSSVKVSCKASGHDFRLTYIHWVRQAPGQGLEWMGRVYAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVEDRVVWHQQKPGKAPKALIYSSSHRYKGVPSRFSGSGSGTEFTLTISSLQPEDFATYFCQQFKSYPLTFGQGTKLEIK

SEQ ID NO: 1333 - protein, 246 aa, synthetic construct
QVQLVQSGAEVKKPGSSVKVSCKASGHDFKLTYIHWVRQAPGQGLEWMGRVSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIK

SEQ ID NO: 1334 - protein, 246 aa, synthetic construct
QVQLVQSGAEVKKPGSSVKVSCKASGHDFKLTYIHWVRQAPGQGLEWMGRISAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGDRVVWHQQKPGKAPKALIYSSSHRYKGVPSRFSGSGSGTEFTLTISSLQPEDFATYFCQQFKSYPLTFGQGTKLEIK

SEQ ID NO: 1335 - protein, 246 aa, synthetic construct
QVQLVQSGAEVKKPGSSVKVSCKASGHDFKLTYIHWVRQAPGQGLEWMGRIYAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGDRVVWHQQKPGKAPKALIYSSSHRYKGVPSRFSGSGSGTEFTLTISSLQPEDFATYFCQQFKSYPLTFGQGTKLEIK

SEQ ID NO: 1336 - protein, 246 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 1337 - protein, 246 aa, synthetic construct
QVQLVQSGAEVKKPGSSVKVSCKASGHDFRLTYIHWVRQAPGQGLEWMGRISAGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 1338 - protein, 243 aa, synthetic construct
EVQLVESGGGLVQPGGSLRLSCAASGFTFSNFGMHWVRQAPGKGLEWVSYISSGSSTIYYADTLKGRFTISRDNSKNTLYLQMNSLRAEDTAVYYCARRGEGAMDYWGQGTTVTVSSGGGGSGGGGSGGGGSGGGGSDNQLTQSPSSLSASVGDRVTITCRASSSVNYIYWYQQKPGKAPKLLIYYTSNLAPGVPSRFSGSGSGNDYTLTISSLQPEDFATYYCQQFTSSPFTFGQGTKLEIK

SEQ ID NO: 1339 - protein, 242 aa, synthetic construct
EVQLVESGGGLVQPGGSLRLSCAASGFTFSNFGMHWVRQAPGKGLEWVSYISSGSSTIYYADTLKGRFTISRDNSKNTLYLQMNSLRAEDTAVYYCARRGEGAMDYWGQGTTVTVSSGGGGSGGGGSGGGGSGGGGSSNELTQPPSVSVSPGQTARITCRASSSVNYIYWYQQKSGQAPVLVIYYTSNLAPGIPERFSGSGSGNMYTLTISGAQVEDEADYYCQQFTSSPFTFGTGTKVTVL

SEQ ID NO: 1340 - protein, 243 aa, synthetic construct
QVQLVESGGGVVQPGRSLRLSCAASGFTFSNFGMHWVRQAPGKGLEWVAYISSGSSTIYYADTLKGRFTISRDNSKNTLYLQMNSLRAEDTAVYYCARRGEGAMDYWGQGTTVTVSSGGGGSGGGGSGGGGSGGGGSDNQLTQSPSFLSASVGDRVTITCRASSSVNYIYWYQQKPGKAPKLLIYYTSNLAPGVPSRFSGSGSGNEYTLTISSLQPEDFATYYCQQFTSSPFTFGQGTKLEIK

SEQ ID NO: 1341 - protein, 243 aa, synthetic construct
QVQLVESGGGVVQPGRSLRLSCAASGFTFSNFGMHWVRQAPGKGLEWVAYISSGSSTIYYADTLKGRFTISRDNSKNTLYLQMNSLRAEDTAVYYCARRGEGAMDYWGQGTTVTVSSGGGGSGGGGSGGGGSGGGGSDNQLTQSPSSLSASVGDRVTITCRASSSVNYIYWYQQKPGKAPKLLIYYTSNLAPGVPSRFSGSGSGNDYTLTISSLQPEDFATYYCQQFTSSPFTFGQGTKLEIK

SEQ ID NO: 1342 - protein, 242 aa, synthetic construct
QVQLVESGGGVVQPGRSLRLSCAASGFTFSNFGMHWVRQAPGKGLEWVAYISSGSSTIYYADTLKGRFTISRDNSKNTLYLQMNSLRAEDTAVYYCARRGEGAMDYWGQGTTVTVSSGGGGSGGGGSGGGGSGGGGSSNELTQPPSVSVSPGQTARITCRASSSVNYIYWYQQKSGQAPVLVIYYTSNLAPGIPERFSGSGSGNMYTLTISGAQVEDEADYYCQQFTSSPFTFGTGTKVTVL

SEQ ID NO: 1343 - skipped

SEQ ID NO: 1344 - protein, 119 aa, synthetic construct
QVQLVQSGAEVKKPGSSVKVSCKASGHDFRLTYIHWVRQAPGQGLEWMGRVSAGSGNTKYNEKFKGRVTITADTSTSTAYMELSSLRSEDTAVYYCAVSYYSYDVLDYWGQGTTVTVSS

SEQ ID NO: 1345 - skipped

SEQ ID NO: 1346 - protein, 119 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSS

SEQ ID NO: 1347 - protein, 107 aa, synthetic construct
DIQMTQSPSFLSASVGDRVTITCKASQNVGNRVVWHQQKPGKAPKALIYSSSHRYKGVPSRFSGSGSGTEFTLTISSLQPEDFATYFCQQFKSYPLTFGQGTKLEIK

SEQ ID NO: 1348 - protein, 119 aa, synthetic construct
QVQLVQSGAEVKKPGSSVKVSCKASGHDFRLTYIHWVRQAPGQGLEWMGRIYAGSGNVKYNEKFKGRVTITADTSTSTAYMELSSLRSEDTAVYYCAVSYYSYDVLDYWGQGTTVTVSS

SEQ ID NO: 1349 - protein, 107 aa, synthetic construct
DIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIK

SEQ ID NO: 1350 - protein, 119 aa, synthetic construct
QVQLVQSGAEVKKPGSSVKVSCKASGHDFKLTYIHWVRQAPGQGLEWMGRVSAGSGNTKYNEKFKGRVTITADTSTSTAYMELSSLRSEDTAVYYCAVSYYSYDVLDYWGQGTTVTVSS

SEQ ID NO: 1351 - protein, 107 aa, synthetic construct; identical to SEQ ID NO: 160

SEQ ID NO: 1352 - skipped

SEQ ID NO: 1353 - protein, 107 aa, synthetic construct; identical to SEQ ID NO: 199

SEQ ID NO: 1354-1355 - skipped (2 entries)

SEQ ID NO: 1356 - protein, 119 aa, synthetic construct
QVQLVQSGAEVKKPGSSVKVSCKASGHDFRLTYIHWVRQAPGQGLEWMGRVYAGSGNTKYNEKFKGRVTITADTSTSTAYMELSSLRSEDTAVYYCAVSYYSYDVLDYWGQGTTVTVSS

SEQ ID NO: 1357 - protein, 107 aa, synthetic construct
DIQMTQSPSFLSASVGDRVTITCKASQNVEDRVVWHQQKPGKAPKALIYSSSHRYKGVPSRFSGSGSGTEFTLTISSLQPEDFATYFCQQFKSYPLTFGQGTKLEIK

SEQ ID NO: 1358-1359 - skipped (2 entries)

SEQ ID NO: 1360 - protein, 119 aa, synthetic construct
QVQLVQSGAEVKKPGSSVKVSCKASGHDFKLTYIHWVRQAPGQGLEWMGRISAGSGNTKYNEKFKGRVTITADTSTSTAYMELSSLRSEDTAVYYCAVSYYSYDVLDYWGQGTTVTVSS

SEQ ID NO: 1361 - protein, 107 aa, synthetic construct
DIQMTQSPSFLSASVGDRVTITCKASQNVGDRVVWHQQKPGKAPKALIYSSSHRYKGVPSRFSGSGSGTEFTLTISSLQPEDFATYFCQQFKSYPLTFGQGTKLEIK

SEQ ID NO: 1362 - protein, 119 aa, synthetic construct
QVQLVQSGAEVKKPGSSVKVSCKASGHDFKLTYIHWVRQAPGQGLEWMGRIYAGSGNTKYNEKFKGRVTITADTSTSTAYMELSSLRSEDTAVYYCAVSYYSYDVLDYWGQGTTVTVSS

SEQ ID NO: 1363-1364 - skipped (2 entries)

SEQ ID NO: 1365 - protein, 107 aa, synthetic construct
DIQMTQSPSFLSASVGDRVTITCRASQNVDNRLGWHQQKPGKAPKALIYSSSHRYKGVPSRFSGSGSGTEFTLTISSLQPEDFATYFCQQFKSYPLTFGQGTKLEIK

SEQ ID NO: 1366 - skipped

SEQ ID NO: 1367 - protein, 107 aa, synthetic construct
DIQMTQSPSFLSASVGDRVTITCKASQNVDNRVAWHQQKPGKAPKALIYAASSLQKGVPSRFSGSGSGTEFTLTISSLQPEDFATYFCQQFKSYPLTFGQGTKLEIK

SEQ ID NO: 1368 - skipped

SEQ ID NO: 1369 - protein, 107 aa, synthetic construct
DIQMTQSPSFLSASVGDRVTITCKASQNVDNRVAWHQQKPGKAPKALIYSSSHRYKGVPSRFSGSGSGTEFTLTISSLQPEDFATYFCLQHNSYPLTFGQGTKLEIK

SEQ ID NO: 1370 - protein, 119 aa, synthetic construct
QVQLVQSGAEVKKPGSSVKVSCKASGHDFKLTYIHWVRQAPGQGLEWMGRVSAGSGNVNYAQKFQGRVTITADTSTSTAYMELSSLRSEDTAVYYCAVSYYSYDVLDYWGQGTTVTVSS

SEQ ID NO: 1371-1375 - skipped (5 entries)

SEQ ID NO: 1376 - protein, 252 aa, synthetic construct
QVQLQQSGTELMKPGASVKISCKASGYTFSNYWIEWIKQRPGHGLEWVGEILPGAGPTNYNEKFKGKATFTADSSSNTAYMQLSSLTSEDSAVYYCARTDYDYDWFAYWGQGTLVTVSAGGGGSGGGGSGGGGSGGGGSDIVMSQSPSSLAVSVGEKVTMSCKSSQSLLYSGNQKNYLAWYQQKPGQSPKLLIYWASTRESGVPDRFTGSGSGTDFTLTINSVKAEDLTVYYCQQYYGYPRTFGGGTKVEIK

SEQ ID NO: 1377-2169 - skipped (793 entries)

SEQ ID NO: 2170 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2171-2179 - skipped (9 entries)

SEQ ID NO: 2180 - protein, 77 aa, synthetic construct
MAPRRARGCRTLGLPALLLLLLLRPPATRGITCPPPMSVEHADIWVKSYSLYSRERYICNSGFKRKAGTSSLTECVL

SEQ ID NO: 2181-2189 - skipped (9 entries)

SEQ ID NO: 2190 - protein, 20 aa, synthetic construct
SGGSGGGGSGGGSGGGGSLQ

SEQ ID NO: 2191 - protein, 133 aa, Homo sapiens [sequence omitted]

SEQ ID NO: 2192 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2193 - protein, 133 aa, Homo sapiens [sequence omitted]

SEQ ID NO: 2194 - protein, 138 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRG

SEQ ID NO: 2195-2269 - skipped (75 entries)

SEQ ID NO: 2270 - protein, 133 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 2271-2279 - skipped (9 entries)

SEQ ID NO: 2280 - protein, 133 aa, Homo sapiens
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 2281-2289 - skipped (9 entries)

SEQ ID NO: 2290 - protein, 518 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2291-2299 - skipped (9 entries)

SEQ ID NO: 2300 - protein, 340 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2301-2309 - skipped (9 entries)

SEQ ID NO: 2310 - protein, 166 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 2311-2319 - skipped (9 entries)

SEQ ID NO: 2320 - protein, 114 aa, Homo sapiens
NWVNVISDLKKIEDLIQSMHIDATLYTESDVHPSCKVTAMKCFLLELQVISLASGDASIHDTVENLIILANNSLSSNGAVTESGCKECEELEEKNIKEFLQSFVHIVQMFINTS

SEQ ID NO: 2321-2999 - skipped (679 entries)

SEQ ID NO: 3000 - protein, 107 aa, synthetic construct
DIQMTQSPSFLSASVGDRVTITCSASQGISNYLNWYQQKPGKAVKLLIYYTSSLHSGVPSRFSGSGSGTEYTLTISSLQPEDFATYYCQQYSKLPRTFGGGTKVEIK

SEQ ID NO: 3001 - protein, 107 aa, synthetic construct
DIQMTQSPSSLSASVGDRVTITCSASQGISNYLNWYQQKPGKAVKLLIYYTSSLHSGVPSRFSGSGSGTDYTLTISSLQPEDFATYYCQQYSKLPRTFGGGTKVEIK

SEQ ID NO: 3002 - protein, 107 aa, synthetic construct
DIQMTQSPSSLSASVGDRVTITCSASQGISNYLNWYQQKPGKVVKLLIYYTSSLHSGVPSRFSGSGSGTDYTLTISSLQPEDVATYYCQQYSKLPRTFGGGTKVEIK

SEQ ID NO: 3003 - protein, 107 aa, synthetic construct
DIQMTQSPSSLSASVGDRVTITCSASQGISNYLNWYQQKPGQAVKLLIYYTSSLHSGVPSRFSGSGSGTDYTLTISSLQPEDVATYYCQQYSKLPRTFGGGTKVEIK

SEQ ID NO: 3004 - protein, 107 aa, synthetic construct
DIQMTQSPSSLSASVGDRVTITCSASQGISNYLNWYQQKPGKAVKLLIYYTSSLHSGVPSRFSGSGSGTDYTFTISSLQPEDIATYYCQQYSKLPRTFGGGTKVEIK

SEQ ID NO: 3005 - protein, 107 aa, synthetic construct
DIQMTQSPSSLSASVGDRVTITCSASQGISNYLNWYQQKPGKTVKLLIYYTSSLHSGIPSRFSGSGSGTDYTLTIRSLQPEDFATYYCQQYSKLPRTFGGGTKVEIK

SEQ ID NO: 3006 - protein, 107 aa, synthetic construct
AIQMTQSPSSLSASVGDRVTITCSASQGISNYLNWYQQKPGKAVKLLIYYTSSLHSGVPSRFSGSGSGTDYTLTISSLQPEDFATYYCQQYSKLPRTFGGGTKVEIK

SEQ ID NO: 3007 - protein, 107 aa, synthetic construct
DIQMTQSPSSVSASVGDRVTITCSASQGISNYLNWYQQKPGKAVKLLIYYTSSLHSGVPSRFSGSGSGTDYTLTISSLQPEDFATYYCQQYSKLPRTFGGGTKVEIK

SEQ ID NO: 3008 - protein, 107 aa, synthetic construct
DIQMTQSPSSLSASVGDRVTITCSASQGISNYLNWYQQKPGKAVKRLIYYTSSLHSGVPSRFSGSGSGTEYTLTISNLQPEDFATYYCQQYSKLPRTFGGGTKVEIK

SEQ ID NO: 3009 - protein, 107 aa, synthetic construct
AIRMTQSPFSLSASVGDRVTITCSASQGISNYLNWYQQKPAKAVKLFIYYTSSLHSGVPSRFSGSGSGTDYTLTISSLQPEDFATYYCQQYSKLPRTFGGGTKVEIK

SEQ ID NO: 3010 - protein, 107 aa, synthetic construct
DIQMTQSPSSLSASVGDRVTITCSASQGISNYLNWYQQKPGKAVKRLIYYTSSLHSGVPSRFSGSGSGTEYTLTISSLQPEDFATYYCQQYSKLPRTFGGGTKVEIK

SEQ ID NO: 3011 - protein, 107 aa, synthetic construct
DIQMTQSPSTLSASVGDRVTITCSASQGISNYLNWYQQKPGKAVKLLIYYTSSLHSGVPSRFSGSGSGTEYTLTISSLQPDDFATYYCQQYSKLPRTFGGGTKVEIK

SEQ ID NO: 3012 - protein, 107 aa, synthetic construct
DIQMTQSPSSLSASVGDRVTITCSASQGISNYLNWYQQKPGKAVKSLIYYTSSLHSGVPSRFSGSGSGTDYTLTISSLQPEDFATYYCQQYSKLPRTFGGGTKVEIK

SEQ ID NO: 3013 - protein, 107 aa, synthetic construct
DIQMTQSPSSLSASVGDRVTITCSASQGISNYLNWYQQKPGKAVKSLIYYTSSLHSGVPSKFSGSGSGTDYTLTISSLQPEDFATYYCQQYSKLPRTFGGGTKVEIK

SEQ ID NO: 3014 - protein, 107 aa, synthetic construct
DIQMTQSPSSLSASVGDRVTITCSASQGISNYLNWYQQKPEKAVKSLIYYTSSLHSGVPSRFSGSGSGTDYTLTISSLQPEDFATYYCQQYSKLPRTFGGGTKVEIK

SEQ ID NO: 3015 - protein, 107 aa, synthetic construct
DIQMTQSPSAMSASVGDRVTITCSASQGISNYLNWYQQKPGKVVKRLIYYTSSLHSGVPSRFSGSGSGTEYTLTISSLQPEDFATYYCQQYSKLPRTFGGGTKVEIK

SEQ ID NO: 3016 - protein, 107 aa, synthetic construct
DIVMTQSPDSLAVSLGERATINCSASQGISNYLNWYQQKPGQPVKLLIYYTSSLHSGVPDRFSGSGSGTDYTLTISSLQAEDVAVYYCQQYSKLPRTFGGGTKVEIK

SEQ ID NO: 3017 - protein, 107 aa, synthetic construct
EIVMTQSPGTLSLSPGERATLSCSASQGISNYLNWYQQKPGQAVKLLIYYTSSLHSGIPDRFSGSGSGTDYTLTISRLEPEDFAVYYCQQYSKLPRTFGGGTKVEIK

SEQ ID NO: 3018 - protein, 107 aa, synthetic construct
EIVMTQSPPTLSLSPGERVTLSCSASQGISNYLNWYQQKPGQAVKLLIYYTSSLHSGIPARFSGSGSGTDYTLTISSLQPEDFAVYYCQQYSKLPRTFGGGTKVEIK

SEQ ID NO: 3019 - protein, 107 aa, synthetic construct
EIVMTQSPPTLSLSPGERVTLSCSASQGISNYLNWYQQKPGQAVKLLIYYTSSLHSSIPARFSGSGSGTDYTLTISSLQPEDFAVYYCQQYSKLPRTFGGGTKVEIK

SEQ ID NO: 3020 - protein, 107 aa, synthetic construct
EIVMTQSPATLSLSPGERATLSCSASQGISNYLNWYQQKPGQAVKLLIYYTSSLHSGIPARFSGSGSGTDYTLTISSLEPEDFAVYYCQQYSKLPRTFGGGTKVEIK

SEQ ID NO: 3021 - protein, 107 aa, synthetic construct
EIVMTQSPATLSLSPGERATLSCSASQGISNYLNWYQQKPGQAVKLLIYYTSSLHSGIPARFSGSGSGTDYTLTISRLEPEDFAVYYCQQYSKLPRTFGGGTKVEIK

SEQ ID NO: 3022 - protein, 107 aa, synthetic construct
EIVMTQSPATLSLSPGERATLSCSASQGISNYLNWYQQKPGQAVKLLIYYTSSLHSGIPDRFSGSGSGTDYTLTISRLEPEDFAVYYCQQYSKLPRTFGGGTKVEIK

SEQ ID NO: 3023 - protein, 107 aa, synthetic construct
EIVMTQSPATLSLSPGERATLSCSASQGISNYLNWYQQKPGLAVKLLIYYTSSLHSGIPDRFSGSGSGTDYTLTISRLEPEDFAVYYCQQYSKLPRTFGGGTKVEIK

SEQ ID NO: 3024 - protein, 107 aa, synthetic construct
DIQMIQSPSFLSASVGDRVSIICSASQGISNYLNWYLQKPGKSVKLFIYYTSSLHSGVSSRFSGRGSGTDYTLTIISLKPEDFAAYYCQQYSKLPRTFGGGTKVEIK

SEQ ID NO: 3025 - protein, 107 aa, synthetic construct
EIVMTQSPATLSLSPGERATLSCSASQGISNYLNWYQQKPGQAVKLLIYYTSSLHSGIPARFSGSGSGTDYTLTISSLQPEDFAVYYCQQYSKLPRTFGGGTKVEIK

SEQ ID NO: 3026 - protein, 107 aa, synthetic construct
EIVMTQSPATLSLSPGERATLSCSASQGISNYLNWYQQKPGQAVKLLIYYTSSLHSGIPARFSGSGPGTDYTLTISSLEPEDFAVYYCQQYSKLPRTFGGGTKVEIK

SEQ ID NO: 3027 - protein, 107 aa, synthetic construct
DIVMTQTPLSLSVTPGQPASISCSASQGISNYLNWYLQKPGQSVKLLIYYTSSLHSGVPDRFSGSGSGTDYTLKISRVEAEDVGVYYCQQYSKLPRTFGGGTKVEIK

SEQ ID NO: 3028 - protein, 107 aa, synthetic construct
DIVMTQTPLSLSVTPGQPASISCSASQGISNYLNWYLQKPGQPVKLLIYYTSSLHSGVPDRFSGSGSGTDYTLKISRVEAEDVGVYYCQQYSKLPRTFGGGTKVEIK

SEQ ID NO: 3029 - protein, 107 aa, synthetic construct
DIVMTQSPAFLSVTPGEKVTITCSASQGISNYLNWYQQKPDQAVKLLIYYTSSLHSGVPSRFSGSGSGTDYTFTISSLEAEDAATYYCQQYSKLPRTFGGGTKVEIK

SEQ ID NO: 3030 - protein, 107 aa, synthetic construct
DIVMTQSPLSLPVTPGEPASISCSASQGISNYLNWYLQKPGQSVKLLIYYTSSLHSGVPDRFSGSGSGTDYTLKISRVEAEDVGVYYCQQYSKLPRTFGGGTKVEIK

SEQ ID NO: 3031 - protein, 107 aa, synthetic construct
DIVMTQTPLSLPVTPGEPASISCSASQGISNYLNWYLQKPGQSVKLLIYYTSSLHSGVPDRFSGSGSGTDYTLKISRVEAEDVGVYYCQQYSKLPRTFGGGTKVEIK

SEQ ID NO: 3032 - protein, 107 aa, synthetic construct
EIVMTQSPATLSVSPGERATLSCSASQGISNYLNWYQQKPGQAVKLLIYYTSSLHSGIPARFSGSGSGTEYTLTISILQSEDFAVYYCQQYSKLPRTFGGGTKVEIK

SEQ ID NO: 3033 - protein, 107 aa, synthetic construct
EIVMTQSPATLSVSPGERATLSCSASQGISNYLNWYQQKPGQAVKLLIYYTSSLHSGIPARFSGSGSGTEYTLTISSLQSEDFAVYYCQQYSKLPRTFGGGTKVEIK

SEQ ID NO: 3034 - protein, 107 aa, synthetic construct
DIVMTQSPLSLPVTLGQPASISCSASQGISNYLNWYQQRPGQSVKRLIYYTSSLHSGVPDRFSGSGSGTDYTLKISRVEAEDVGVYYCQQYSKLPRTFGGGTKVEIK

SEQ ID NO: 3035 - protein, 107 aa, synthetic construct
EITMTQSPAFMSATPGDKVNISCSASQGISNYLNWYQQKPGEAVKFIIYYTSSLHSGIPPRFSGSGYGTDYTLTINNIESEDAAYYYCQQYSKLPRTFGGGTKVEIK

SEQ ID NO: 3036 - protein, 107 aa, synthetic construct
DIVMTQTPLSSPVTLGQPASISCSASQGISNYLNWYQQRPGQPVKLLIYYTSSLHSGVPDRFSGSGAGTDYTLKISRVEAEDVGVYYCQQYSKLPRTFGGGTKVEIK

SEQ ID NO: 3037 - protein, 107 aa, synthetic construct
EIVMTQSPDFQSVTPKEKVTITCSASQGISNYLNWYQQKPDQSVKLLIYYTSSLHSGVPSRFSGSGSGTDYTLTINSLEAEDAATYYCQQYSKLPRTFGGGTKVEIK

SEQ ID NO: 3038 - protein, 107 aa, synthetic construct
EIVMTQTPLSLSITPGEQASISCSASQGISNYLNWYLQKARPVVKLLIYYTSSLHSGVPDRFSGSGSGTDYTLKISRVEAEDFGVYYCQQYSKLPRTFGGGTKVEIK

SEQ ID NO: 3039 - protein, 107 aa, synthetic construct
EIVMTQTPLSLSITPGEQASMSCSASQGISNYLNWYLQKARPVVKLLIYYTSSLHSGVPDRFSGSGSGTDYTLKISRVEAEDFGVYYCQQYSKLPRTFGGGTKVEIK

SEQ ID NO: 3040 - protein, 120 aa, synthetic construct
QVTLKESGPVLVKPTETLTLTCTVSGFSLTAYGVNWVRQPPGKALEWLGMIWGDGNTDYNSALKSRLTISKDNSKSQVVLTMTNMDPVDTATYYCARDRVTATLYAMDYWGQGTLVTVSS

SEQ ID NO: 3041 - protein, 120 aa, synthetic construct
QVTLKESGPALVKPTETLTLTCTVSGFSLTAYGVNWVRQPPGKALEWLGMIWGDGNTDYNSALKSRLIISKDNSKSQVVLTMTNMDPVDTATYYCARDRVTATLYAMDYWGQGTLVTVSS

SEQ ID NO: 3042 - protein, 120 aa, synthetic construct
QVTLKESGPALVKPTQTLTLTCTVSGFSLTAYGVNWVRQPPGKALEWLGMIWGDGNTDYNSALKSRLTISKDNSKSQVVLTMTNMDPVDTATYYCARDRVTATLYAMDYWGQGTLVTVSS

SEQ ID NO: 3043 - protein, 120 aa, synthetic construct
QVQLQESGPGLVKPSGTLSLTCAVSGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 3044 - protein, 120 aa, synthetic construct
QVTLKESGPTLVKPTQTLTLTCTVSGFSLTAYGVNWVRQPPGKALEWLGMIWGDGNTDYNSALKSRLTITKDNSKSQVVLTMTNMDPVDTATYYCARDRVTATLYAMDYWGQGTLVTVSS

SEQ ID NO: 3045 - protein, 120 aa, synthetic construct
QVTLKESGPALVKPTQTLTLTCTVSGFSLTAYGVNWVRQPPGKALEWLGMIWGDGNTDYNSALKSRLTITKDNSKSQVVLTMTNMDPVDTATYYCARDRVTATLYAMDYWGQGTLVTVSS

SEQ ID NO: 3046 - protein, 120 aa, synthetic construct
QVQLQESGPGLVKPSQTLSLTCTVSGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 3047 - protein, 120 aa, synthetic construct
QVQLQESGPGLVKPSETLSLTCTVSGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 3048 - protein, 120 aa, synthetic construct
QVQLQESGPGLVKPSQTLSLTCAVSGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 3049 - protein, 120 aa, synthetic construct
QVQLQESGPGLVKPSDTLSLTCTVSGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 3050 - protein, 120 aa, synthetic construct
QVQLQESGPGLVKPSQTLSLTCTVSGFSLTAYGVNWVRQHPGKGLEWLGMIWGDGNTDYNSALKSRLTISKDNSKSQVSLKLSSVTAADTAVYYCARDRVTATLYAMDYWGQGTLVTVSS

SEQ ID NO: 3051 - protein, 120 aa, synthetic construct
QVQLQESGPGLVKPSQTLSLTCTVSGFSLTAYGVNWVRQPAGKGLEWLGMIWGDGNTDYNSALKSRLTISKDNSKSQVSLKLSSVTAADTAVYYCARDRVTATLYAMDYWGQGTLVTVSS

SEQ ID NO: 3052 - protein, 120 aa, synthetic construct
QVQLQESGPGLVKPSQTLSLTCAVSGFSLTAYGVNWVRQPPGKGLEWLGMIWGDGNTDYNSALKSRLTISKDNSKSQVSLKLSSVTAVDTAVYYCARDRVTATLYAMDYWGQGTLVTVSS

SEQ ID NO: 3053 - protein, 120 aa, synthetic construct
QVQLQESGPGLVKPSETLSLTCTVSGFSLTAYGVNWVRQPPGKGLEWLGMIWGDGNTDYNSALKSRLTISKDNSKSHVSLKLSSVTAADTAVYYCARDRVTATLYAMDYWGQGTLVTVSS

SEQ ID NO: 3054 - protein, 120 aa, synthetic construct
QVQLQESGPGLVKPSETLSLTCAVSGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 3055 - protein, 120 aa, synthetic construct
QVQLQESGPGLVKPSQTLSLTCAVYGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 3056 - protein, 120 aa, synthetic construct
RVQLQESGPGLVKPSETLSLTCTVSGFSLTAYGVNWVRQPPGKGLEWLGMIWGDGNTDYNSALKSRLTISKDNSKSQVPLKLSSVTAADTAVYYCARDRVTATLYAMDYWGQGTLVTVSS

SEQ ID NO: 3057 - protein, 120 aa, synthetic construct
QVQLQESGPGLVKPSQTLSLTCTVSGFSLTAYGVNWVRQHPGKGLEWLGMIWGDGNTDYNSALKSLLTISKDNSKSQVSLKLSSVTAADTAVYYCARDRVTATLYAMDYWGQGTLVTVSS

SEQ ID NO: 3058 - protein, 120 aa, synthetic construct
QVQLQESGPGLVKPSDTLSLTCAVSGFSLTAYGVNWVRQPPGKGLEWLGMIWGDGNTDYNSALKSRLTISKDNSKSQVSLKLSSVTALDTAVYYCARDRVTATLYAMDYWGQGTLVTVSS

SEQ ID NO: 3059 - protein, 120 aa, synthetic construct
QVQLQESGPGLVKPSDTLSLTCAVSGFSLTAYGVNWVRQPPGKGLEWLGMIWGDGNTDYNSALKSRLTISKDNSKSQVSLKLSSVTAVDTAVYYCARDRVTATLYAMDYWGQGTLVTVSS

SEQ ID NO: 3060 - protein, 120 aa, synthetic construct
QVQLQESGSGLVKPSQTLSLTCAVSGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 3061 - protein, 120 aa, synthetic construct
EVQLVESGGGLVQPGRSLRLSCTVSGFSLTAYGVNWVRQAPGKGLEWLGMIWGDGNTDYNSALKSRLTISKDNSKSIVYLQMNSLKTEDTAVYYCARDRVTATLYAMDYWGQGTLVTVSS

SEQ ID NO: 3062 - protein, 120 aa, synthetic construct
EVQLVESGGGLVQPGPSLRLSCTVSGFSLTAYGVNWVRQAPGKGLEWLGMIWGDGNTDYNSALKSRLTISKDNSKSIVYLQMNSLKTEDTAVYYCARDRVTATLYAMDYWGQGTLVTVSS

SEQ ID NO: 3063 - protein, 120 aa, synthetic construct
QVQLQESGSGLVKPSQTLSLTCAVSGFSLTAYGVNWVRQSPGKGLEWLGMIWGDGNTDYNSALKSRLTISKDNSKSQVSLKLSSVTAADTAVYYCARDRVTATLYAMDYWGQGTLVTVSS

SEQ ID NO: 3064 - protein, 120 aa, synthetic construct
QVQLQESGPGLVKPSETLSLTCTVSGFSLTAYGVNWVRQPAGKGLEWLGMIWGDGNTDYNSALKSRLTISKDNSKSQVSLKLSSVTAADTAVYYCARDRVTATLYAMDYWGQGTLVTVSS

SEQ ID NO: 3065 - protein, 120 aa, synthetic construct
EVQLVESGGGLVKPGRSLRLSCTVSGFSLTAYGVNWVRQAPGKGLEWLGMIWGDGNTDYNSALKSRLTISKDNSKSIVYLQMNSLKTEDTAVYYCARDRVTATLYAMDYWGQGTLVTVSS

SEQ ID NO: 3066 - protein, 120 aa, synthetic construct
QVQLQESGPGLVKPSETLSLTCAVYGFSLTAYGVNWVRQPPGKGLEWLGMIWGDGNTDYNSALKSRLTISKDNSKSQVYLKLSSVTAADTAVYYCARDRVTATLYAMDYWGQGTLVTVSS

SEQ ID NO: 3067 - protein, 120 aa, synthetic construct
QVQLQESGPGLVKPSDTLSLTCAVSGFSLTAYGVNWVRQPPGKGLEWLGMIWGDGNTDYNSALKSRLTISKDNSKSQVSLKLSSVTAVDTGVYYCARDRVTATLYAMDYWGQGTLVTVSS

SEQ ID NO: 3068 - protein, 120 aa, synthetic construct
EVQLVESGGGLVQPGGSLRLSCAVSGFSLTAYGVNWVRQAPGKGLEWLGMIWGDGNTDYNSALKSRLTISKDNSKSSVYLQMNSLKTEDTAVYYCARDRVTATLYAMDYWGQGTLVTVSS

SEQ ID NO: 3069 - protein, 120 aa, synthetic construct
EVQLVESGGGLVKPGGSLRLSCAVSGFSLTAYGVNWVRQAPGKGLEWLGMIWGDGNTDYNSALKSRLTISKDNSKSTVYLQMNSLKTEDTAVYYCARDRVTATLYAMDYWGQGTLVTVSS

SEQ ID NO: 3070 - protein, 120 aa, synthetic construct
QVQLQQSGPGLVKPSQTLSLTCAVSGFSLTAYGVNWVRQSPSRGLEWLGMIWGDGNTDYNSALKSRLTINKDNSKSQVSLQLNSVTPEDTAVYYCARDRVTATLYAMDYWGQGTLVTVSS

SEQ ID NO: 3071 - protein, 120 aa, synthetic construct
QVQLVESGGGLVQPGGSLRLSCSVSGFSLTAYGVNWVRQAPGKGLEWLGMIWGDGNTDYNSALKSRLTISKDNSKSTVYLQMNSLRAEDTAVYYCARDRVTATLYAMDYWGQGTLVTVSS

SEQ ID NO: 3072 - protein, 120 aa, synthetic construct
QVQLQQWGAGLLKPSETLSLTCAVYGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 3073 - protein, 120 aa, synthetic construct
QVQLVESGGGVVQPGRSLRLSCAVSGFSLTAYGVNWVRQAPGKGLEWLGMIWGDGNTDYNSALKSRLTISKDNSTSTVFLQMNSLRAEDTAVYYCARDRVTATLYAMDYWGQGTLVTVSS

SEQ ID NO: 3074 - protein, 120 aa, synthetic construct
EVQLVESGGGLVQPGGSLRLSCAVSGFSLTAYGVNWVRQAPGKGLEWLGMIWGDGNTDYNSALKSRLTISKDNSKSTVYLQMNSLRAEDTAVYYCARDRVTATLYAMDYWGQGTLVTVSS

SEQ ID NO: 3075 - protein, 120 aa, synthetic construct
EVQLVESGGGLVQPGGSLRLSCAVSGFSLTAYGVNWVRQAPGKGLEWLGMIWGDGNTDYNSALKSRLTISKDNAKSSVYLQMNSLRDEDTAVYYCARDRVTATLYAMDYWGQGTLVTVSS

SEQ ID NO: 3076 - protein, 120 aa, synthetic construct
EVQLLESGGGLVQPGGSLRLSCAVSGFSLTAYGVNWVRQAPGKGLEWLGMIWGDGNTDYNSALKSRLTISKDNSKSTVYLQMNSLRAEDTAVYYCARDRVTATLYAMDYWGQGTLVTVSS

SEQ ID NO: 3077 - protein, 120 aa, synthetic construct
QVQLVESGGGLVKPGGSLRLSCAVSGFSLTAYGVNWVRQAPGKGLEWLGMIWGDGNTDYNSALKSRLTISKDNAKSSVYLQMNSLRAEDTAVYYCARDRVTATLYAMDYWGQGTLVTVSS

SEQ ID NO: 3078 - protein, 120 aa, synthetic construct
EVQLVESGGGLVQPGGSLKLSCAVSGFSLTAYGVNWVRQASGKGLEWLGMIWGDGNTDYNSALKSRLTISKDNSKSTVYLQMNSLKTEDTAVYYCARDRVTATLYAMDYWGQGTLVTVSS

SEQ ID NO: 3079 - protein, 120 aa, synthetic construct
QVQLLESGGGLVKPGGSLRLSCAVSGFSLTAYGVNWVRQAPGKGLEWLGMIWGDGNTDYNSALKSRLTISKDNAKSSVYLQMNSLRAEDTAVYYCARDRVTATLYAMDYWGQGTLVTVSS

SEQ ID NO: 3080 - protein, 120 aa, synthetic construct
QVQLVESGGGVVQPGRSLRLSCAVSGFSLTAYGVNWVRQAPGKGLEWLGMIWGDGNTDYNSALKSRLTISKDNSKSTVYLQMNSLRAEDTAVYYCARDRVTATLYAMDYWGQGTLVTVSS

SEQ ID NO: 3081 - protein, 120 aa, synthetic construct
QVQLVESGGGVVQPGRSLRLSCAVSGFSLTAYGVNWVRQAPGKGLEWLGMIWGDGNTDYNSALKSRLTISKDNSKSRVYLQMNSLRAEDTAVYYCARDRVTATLYAMDYWGQGTLVTVSS

SEQ ID NO: 3082 - protein, 120 aa, synthetic construct
QVQLVESGGGVVQPGRSLRLSCAVSGFSLTAYGVNWVRQAPGKGLEWLGMIWGDGNTDYNSALKSRLAISKDNSKSTVYLQMNSLRAEDTAVYYCARDRVTATLYAMDYWGQGTLVTVSS

SEQ ID NO: 3083 - protein, 120 aa, synthetic construct
QVQLVESGGGVVQPGGSLRLSCAVSGFSLTAYGVNWVRQAPGKGLEWLGMIWGDGNTDYNSALKSRLTISKDNSKSTVYLQMNSLRAEDTAVYYCARDRVTATLYAMDYWGQGTLVTVSS

SEQ ID NO: 3084 - protein, 120 aa, synthetic construct
EVQLVESGGGLVQPGGSLRLSCAVSGFSLTAYGVNWVRQAPGKGLEWLGMIWGDGNTDYNSALKSRLTISKDNAKSTVYLQMNSLRAEDTAVYYCARDRVTATLYAMDYWGQGTLVTVSS

SEQ ID NO: 3085 - protein, 120 aa, synthetic construct
EVQLVESGGGLVQPGGSLRLSCAVSGFSLTAYGVNWVRQAPGKGLEWLGMIWGDGNTDYNSALKSRLTISKDNAKSSVYLQMNSLRAEDTAVYYCARDRVTATLYAMDYWGQGTLVTVSS

SEQ ID NO: 3086 - protein, 120 aa, synthetic construct
EVQLVESGGVVVQPGGSLRLSCAVSGFSLTAYGVNWVRQAPGKGLEWLGMIWGDGNTDYNSALKSRLTISKDNSKSSVYLQMNSLRTEDTALYYCARDRVTATLYAMDYWGQGTLVTVSS

SEQ ID NO: 3087 - protein, 120 aa, synthetic construct
EVQLVESGGGLVQPGGSLRLSCAVSGFSLTAYGVNWVRQAPGKGLEWLGMIWGDGNTDYNSALKSRLTISKHNSKSTVYLQMNSLRAEDTAVYYCARDRVTATLYAMDYWGQGTLVTVSS

SEQ ID NO: 3088 - protein, 120 aa, synthetic construct
EVQLVESGGGLVKPGGSLRLSCAVSGFSLTAYGVNWVRQAPGKGLEWLGMIWGDGNTDYNSALKSRLTISKDNAKSSVYLQMNSLRAEDTAVYYCARDRVTATLYAMDYWGQGTLVTVSS

SEQ ID NO: 3089 - protein, 120 aa, synthetic construct
EVQLVESGGGLIQPGGSLRLSCAVSGFSLTAYGVNWVRQPPGKGLEWLGMIWGDGNTDYNSALKSRLTISKDNSKSTVYLQMNSLRAEDTAVYYCARDRVTATLYAMDYWGQGTLVTVSS

SEQ ID NO: 3090 - skipped

SEQ ID NO: 3091 - protein, 119 aa, synthetic construct
QVQLQQSGPELVKPGASVKISCKASGYAFSSSWMNWVKQRPGQGLEWIGRIYPGDGDTKYNGKFKGKATLTADKSSSTAYMHLSSLTSVDSAVYFCARRGTGGWYFDVWGAGTTVTVSS

SEQ ID NO: 3092 - protein, 111 aa, synthetic construct
DIVLTQSPASLAVSLGQRATISCRASESVDSSGNSFMHWYQQKPGQPPQLLIYRASNLESGIPARFSGSGSRTDFTLTINPVEADDVATFYCQQSFDDPFTFGSGTKLEIK

SEQ ID NO: 3093 - protein, 111 aa, synthetic construct
DIVLTQSPDSLAVSLGERATINCRASESVDSSGNSFMHWYQQKPGQPPQLLIYRASNLESGVPDRFSGSGSRTDFTLTISSLQAEDVAVYYCQQSFDDPFTFGQGTKLEIK

SEQ ID NO: 3094 - protein, 111 aa, synthetic construct
EIVLTQSPATLSLSPGERATLSCRASESVDSSGNSFMHWYQQKPGQAPQLLIYRASNLESGIPARFSGSGSRTDFTLTISSLEPEDFAVYYCQQSFDDPFTFGQGTKLEIK

SEQ ID NO: 3095 - protein, 111 aa, synthetic construct
EIVLTQSPATLSLSPGERATLSCRASESVDSSGNSFMHWYQQKPGQAPQLLIYRASNLESGIPARFSGSGSRTDFTLTISRLEPEDFAVYYCQQSFDDPFTFGQGTKLEIK

SEQ ID NO: 3096 - protein, 111 aa, synthetic construct
EIVLTQSPATLSLSPGERATLSCRASESVDSSGNSFMHWYQQKPGQAPQLLIYRASNLESGIPARFSGSGSRTDFTLTISSLQPEDFAVYYCQQSFDDPFTFGQGTKLEIK

SEQ ID NO: 3097 - protein, 111 aa, synthetic construct
DIQLTQSPSSLSASVGDRVTITCRASESVDSSGNSFMHWYQQKPGQAPQLLIYRASNLESGVPSRFSGSGSRTDFTLTISSLQPEDVATYYCQQSFDDPFTFGQGTKLEIK

SEQ ID NO: 3098 - protein, 111 aa, synthetic construct
EIVLTQSPATLSLSPGERATLSCRASESVDSSGNSFMHWYQQKPGQAPQLLIYRASNLESGIPARFSGSGPRTDFTLTISSLEPEDFAVYYCQQSFDDPFTFGQGTKLEIK

SEQ ID NO: 3099 - protein, 111 aa, synthetic construct
EIVLTQSPATLSLSPGERATLSCRASESVDSSGNSFMHWYQQKPGQAPQLLIYRASNLESGIPDRFSGSGSRTDFTLTISRLEPEDFAVYYCQQSFDDPFTFGQGTKLEIK

SEQ ID NO: 3100 - protein, 111 aa, synthetic construct
DIQLTQSPSSLSASVGDRVTITCRASESVDSSGNSFMHWYQQKPGKVPQLLIYRASNLESGVPSRFSGSGSRTDFTLTISSLQPEDVATYYCQQSFDDPFTFGQGTKLEIK

SEQ ID NO: 3101 - protein, 111 aa, synthetic construct
DIQLTQSPSSLSASVGDRVTITCRASESVDSSGNSFMHWYQQKPGKTPQLLIYRASNLESGIPSRFSGSGSRTDFTLTIRSLQPEDFATYYCQQSFDDPFTFGQGTKLEIK

SEQ ID NO: 3102 - protein, 111 aa, synthetic construct
EIVLTQSPGTLSLSPGERATLSCRASESVDSSGNSFMHWYQQKPGQAPQLLIYRASNLESGIPDRFSGSGSRTDFTLTISRLEPEDFAVYYCQQSFDDPFTFGQGTKLEIK

SEQ ID NO: 3103 - protein, 111 aa, synthetic construct
EIVLTQSPATLSLSPGERATLSCRASESVDSSGNSFMHWYQQKPGLAPQLLIYRASNLESGIPDRFSGSGSRTDFTLTISRLEPEDFAVYYCQQSFDDPFTFGQGTKLEIK

SEQ ID NO: 3104 - protein, 111 aa, synthetic construct
DIQLTQSPSSLSASVGDRVTITCRASESVDSSGNSFMHWYQQKPGKAPQLLIYRASNLESGVPSRFSGSGSRTDFTLTISSLQPEDFATYYCQQSFDDPFTFGQGTKLEIK

SEQ ID NO: 3105 - protein, 111 aa, synthetic construct
DIQLTQSPSSVSASVGDRVTITCRASESVDSSGNSFMHWYQQKPGKAPQLLIYRASNLESGVPSRFSGSGSRTDFTLTISSLQPEDFATYYCQQSFDDPFTFGQGTKLEIK

SEQ ID NO: 3106 - protein, 111 aa, synthetic construct
AIQLTQSPSSLSASVGDRVTITCRASESVDSSGNSFMHWYQQKPGKAPQLLIYRASNLESGVPSRFSGSGSRTDFTLTISSLQPEDFATYYCQQSFDDPFTFGQGTKLEIK

SEQ ID NO: 3107 - protein, 111 aa, synthetic construct
DIQLTQSPSFLSASVGDRVTITCRASESVDSSGNSFMHWYQQKPGKAPQLLIYRASNLESGVPSRFSGSGSRTEFTLTISSLQPEDFATYYCQQSFDDPFTFGQGTKLEIK

SEQ ID NO: 3108 - protein, 111 aa, synthetic construct
DIQLTQSPSSLSASVGDRVTITCRASESVDSSGNSFMHWYQQKPGKAPQLLIYRASNLESGVPSRFSGSGSRTDFTFTISSLQPEDIATYYCQQSFDDPFTFGQGTKLEIK

SEQ ID NO: 3109 - protein, 111 aa, synthetic construct
EIVLTQSPATLSVSPGERATLSCRASESVDSSGNSFMHWYQQKPGQAPQLLIYRASNLESGIPARFSGSGSRTEFTLTISILQSEDFAVYYCQQSFDDPFTFGQGTKLEIK

SEQ ID NO: 3110 - protein, 111 aa, synthetic construct
EIVLTQSPATLSVSPGERATLSCRASESVDSSGNSFMHWYQQKPGQAPQLLIYRASNLESGIPARFSGSGSRTEFTLTISSLQSEDFAVYYCQQSFDDPFTFGQGTKLEIK

SEQ ID NO: 3111 - protein, 111 aa, synthetic construct
AIRLTQSPFSLSASVGDRVTITCRASESVDSSGNSFMHWYQQKPAKAPQLFIYRASNLESGVPSRFSGSGSRTDFTLTISSLQPEDFATYYCQQSFDDPFTFGQGTKLEIK

SEQ ID NO: 3112 - protein, 111 aa, synthetic construct
DIQLTQSPSSLSASVGDRVTITCRASESVDSSGNSFMHWYQQKPGKAPQSLIYRASNLESGVPSRFSGSGSRTDFTLTISSLQPEDFATYYCQQSFDDPFTFGQGTKLEIK

SEQ ID NO: 3113 - protein, 111 aa, synthetic construct
DIQLTQSPSSLSASVGDRVTITCRASESVDSSGNSFMHWYQQKPGKAPQRLIYRASNLESGVPSRFSGSGSRTEFTLTISNLQPEDFATYYCQQSFDDPFTFGQGTKLEIK

SEQ ID NO: 3114 - protein, 111 aa, synthetic construct
DIQLTQSPSTLSASVGDRVTITCRASESVDSSGNSFMHWYQQKPGKAPQLLIYRASNLESGVPSRFSGSGSRTEFTLTISSLQPDDFATYYCQQSFDDPFTFGQGTKLEIK

SEQ ID NO: 3115 - protein, 111 aa, synthetic construct
EIVLTQSPDFQSVTPKEKVTITCRASESVDSSGNSFMHWYQQKPDQSPQLLIYRASNLESGVPSRFSGSGSRTDFTLTINSLEAEDAATYYCQQSFDDPFTFGQGTKLEIK

SEQ ID NO: 3116 - protein, 111 aa, synthetic construct
DIQLTQSPSSLSASVGDRVTITCRASESVDSSGNSFMHWYQQKPGKAPQSLIYRASNLESGVPSKFSGSGSRTDFTLTISSLQPEDFATYYCQQSFDDPFTFGQGTKLEIK

SEQ ID NO: 3117 - protein, 111 aa, synthetic construct
DIQLTQSPSSLSASVGDRVTITCRASESVDSSGNSFMHWYQQKPGKAPQRLIYRASNLESGVPSRFSGSGSRTEFTLTISSLQPEDFATYYCQQSFDDPFTFGQGTKLEIK

SEQ ID NO: 3118 - protein, 111 aa, synthetic construct
DIVLTQTPLSLSVTPGQPASISCRASESVDSSGNSFMHWYLQKPGQPPQLLIYRASNLESGVPDRFSGSGSRTDFTLKISRVEAEDVGVYYCQQSFDDPFTFGQGTKLEIK

SEQ ID NO: 3119 - protein, 111 aa, synthetic construct
DIQLTQSPSSLSASVGDRVTITCRASESVDSSGNSFMHWYQQKPEKAPQSLIYRASNLESGVPSRFSGSGSRTDFTLTISSLQPEDFATYYCQQSFDDPFTFGQGTKLEIK

SEQ ID NO: 3120 - protein, 111 aa, synthetic construct
EIVLTQSPPTLSLSPGERVTLSCRASESVDSSGNSFMHWYQQKPGQAPQLLIYRASNLESGIPARFSGSGSRTDFTLTISSLQPEDFAVYYCQQSFDDPFTFGQGTKLEIK

SEQ ID NO: 3121 - protein, 111 aa, synthetic construct
DIQLTQSPSAMSASVGDRVTITCRASESVDSSGNSFMHWYQQKPGKVPQRLIYRASNLESGVPSRFSGSGSRTEFTLTISSLQPEDFATYYCQQSFDDPFTFGQGTKLEIK

SEQ ID NO: 3122 - protein, 111 aa, synthetic construct
DIVLTQSPLSLPVTPGEPASISCRASESVDSSGNSFMHWYLQKPGQSPQLLIYRASNLESGVPDRFSGSGSRTDFTLKISRVEAEDVGVYYCQQSFDDPFTFGQGTKLEIK

SEQ ID NO: 3123 - protein, 111 aa, synthetic construct
DIVLTQTPLSLPVTPGEPASISCRASESVDSSGNSFMHWYLQKPGQSPQLLIYRASNLESGVPDRFSGSGSRTDFTLKISRVEAEDVGVYYCQQSFDDPFTFGQGTKLEIK

SEQ ID NO: 3124 - protein, 111 aa, synthetic construct
DIVLTQTPLSLSVTPGQPASISCRASESVDSSGNSFMHWYLQKPGQSPQLLIYRASNLESGVPDRFSGSGSRTDFTLKISRVEAEDVGVYYCQQSFDDPFTFGQGTKLEIK

SEQ ID NO: 3125 - protein, 111 aa, synthetic construct
EIVLTQSPPTLSLSPGERVTLSCRASESVDSSGNSFMHWYQQKPGQAPQLLIYRASNLESSIPARFSGSGSRTDFTLTISSLQPEDFAVYYCQQSFDDPFTFGQGTKLEIK

SEQ ID NO: 3126 - protein, 111 aa, synthetic construct
DIVLTQSPLSLPVTLGQPASISCRASESVDSSGNSFMHWYQQRPGQSPQRLIYRASNLESGVPDRFSGSGSRTDFTLKISRVEAEDVGVYYCQQSFDDPFTFGQGTKLEIK

SEQ ID NO: 3127 - protein, 111 aa, synthetic construct
DIVLTQTPLSSPVTLGQPASISCRASESVDSSGNSFMHWYQQRPGQPPQLLIYRASNLESGVPDRFSGSGARTDFTLKISRVEAEDVGVYYCQQSFDDPFTFGQGTKLEIK

SEQ ID NO: 3128 - protein, 111 aa, synthetic construct
DIVLTQSPAFLSVTPGEKVTITCRASESVDSSGNSFMHWYQQKPDQAPQLLIYRASNLESGVPSRFSGSGSRTDFTFTISSLEAEDAATYYCQQSFDDPFTFGQGTKLEIK

SEQ ID NO: 3129 - protein, 111 aa, synthetic construct
DIQLIQSPSFLSASVGDRVSIICRASESVDSSGNSFMHWYLQKPGKSPQLFIYRASNLESGVSSRFSGRGSRTDFTLTIISLKPEDFAAYYCQQSFDDPFTFGQGTKLEIK

SEQ ID NO: 3130 - protein, 111 aa, synthetic construct
EIVLTQTPLSLSITPGEQASISCRASESVDSSGNSFMHWYLQKARPVPQLLIYRASNLESGVPDRFSGSGSRTDFTLKISRVEAEDFGVYYCQQSFDDPFTFGQGTKLEIK

SEQ ID NO: 3131 - protein, 111 aa, synthetic construct
EIVLTQTPLSLSITPGEQASMSCRASESVDSSGNSFMHWYLQKARPVPQLLIYRASNLESGVPDRFSGSGSRTDFTLKISRVEAEDFGVYYCQQSFDDPFTFGQGTKLEIK

SEQ ID NO: 3132 - protein, 111 aa, synthetic construct
EITLTQSPAFMSATPGDKVNISCRASESVDSSGNSFMHWYQQKPGEAPQFIIYRASNLESGIPPRFSGSGYRTDFTLTINNIESEDAAYYYCQQSFDDPFTFGQGTKLEIK

SEQ ID NO: 3133 - protein, 119 aa, synthetic construct
QVQLVQSGAEVKKPGASVKVSCKASGYAFSSSWMNWVRQAPGQGLEWIGRIYPGDGDTKYNGKFKGRATLTADKSTSTAYMELSSLRSEDTAVYYCARRGTGGWYFDVWGQGTTVTVSS

SEQ ID NO: 3134 - protein, 119 aa, synthetic construct
QVQLVQSGAEVKKPGSSVKVSCKASGYAFSSSWMNWVRQAPGQGLEWIGRIYPGDGDTKYNGKFKGRATLTADKSTSTAYMELSSLRSEDTAVYYCARRGTGGWYFDVWGQGTTVTVSS

SEQ ID NO: 3135 - protein, 119 aa, synthetic construct
QVQLVQSGAEVKKPGASVKVSCKASGYAFSSSWMNWVRQAPGKGLEWIGRIYPGDGDTKYNGKFKGRATLTADKSTSTAYMELSSLRSEDTAVYYCARRGTGGWYFDVWGQGTTVTVSS

SEQ ID NO: 3136 - protein, 119 aa, synthetic construct
QVQLVQSGAEVKKPGASVKVSCKASGYAFSSSWMNWVRQAPGQELEWIGRIYPGDGDTKYNGKFKGRATLTADKSISTAYMELSSLRSEDTATYYCARRGTGGWYFDVWGQGTTVTVSS

SEQ ID NO: 3137 - protein, 119 aa, synthetic construct
EVQLVQSGAEVKKPGATVKISCKASGYAFSSSWMNWVQQAPGKGLEWIGRIYPGDGDTKYNGKFKGRATLTADKSTSTAYMELSSLRSEDTAVYYCARRGTGGWYFDVWGQGTTVTVSS

SEQ ID NO: 3138 - protein, 119 aa, synthetic construct
QVQLVQSGAEVKKTGSSVKVSCKASGYAFSSSWMNWVRQAPGQALEWIGRIYPGDGDTKYNGKFKGRATLTADKSMSTAYMELSSLRSEDTAMYYCARRGTGGWYFDVWGQGTTVTVSS

SEQ ID NO: 3139 - protein, 119 aa, synthetic construct
QVQLVQSGAEVKKPGASVKVSCKASGYAFSSSWMNWVRQAPGQRLEWIGRIYPGDGDTKYNGKFKGRATLTADKSASTAYMELSSLRSEDMAVYYCARRGTGGWYFDVWGQGTTVTVSS

SEQ ID NO: 3140 - protein, 119 aa, synthetic construct
QVQLVQSGAEVKKPGASVKVSCKASGYAFSSSWMNWVRQAPGQGLEWIGRIYPGDGDTKYNGKFKGRATLTADKSTSTAYMELRSLRSDDMAVYYCARRGTGGWYFDVWGQGTTVTVSS

SEQ ID NO: 3141 - protein, 119 aa, synthetic construct
QVQLVQSGAEVKKPGASVKVSCKASGYAFSSSWMNWVRQAPGQRLEWIGRIYPGDGDTKYNGKFKGRATLTADKSASTAYMELSSLRSEDTAVYYCARRGTGGWYFDVWGQGTTVTVSS

SEQ ID NO: 3142 - protein, 119 aa, synthetic construct
QVQLVQSGAEVKKPGASVKVSCKASGYAFSSSWMNWVRQAPGQGLEWIGRIYPGDGDTKYNGKFKGRATLTADKSTSTAYMELRSLRSDDTAVYYCARRGTGGWYFDVWGQGTTVTVSS

SEQ ID NO: 3143 - protein, 119 aa, synthetic construct
QVQLVQSGAEVKKPGASVKVSCKASGYAFSSSWMNWVRQAPGQGLEWIGRIYPGDGDTKYNGKFKGRATLTADKSISTAYMELSRLRSDDTAVYYCARRGTGGWYFDVWGQGTTVTVSS

SEQ ID NO: 3144 - protein, 119 aa, synthetic construct
QVQLVQSGAEVKKPGASVKVSCKASGYAFSSSWMNWVRQAPGQGLEWIGRIYPGDGDTKYNGKFKGRATLTADKSISTAYMELSRLRSDDTVVYYCARRGTGGWYFDVWGQGTTVTVSS

SEQ ID NO: 3145 - protein, 119 aa, synthetic construct
QVQLVQSGAEVKKPGASVKVSCKASGYAFSSSWMNWVRQAPGQGLEWIGRIYPGDGDTKYNGKFKGWATLTADKSISTAYMELSRLRSDDTAVYYCARRGTGGWYFDVWGQGTTVTVSS

SEQ ID NO: 3146 - protein, 119 aa, synthetic construct
QVQLVQSGAEVKKPGASVKVSCKASGYAFSSSWMNWVRQATGQGLEWIGRIYPGDGDTKYNGKFKGRATLTANKSISTAYMELSSLRSEDTAVYYCARRGTGGWYFDVWGQGTTVTVSS

SEQ ID NO: 3147 - protein, 119 aa, synthetic construct
QVQLVQSGSELKKPGASVKVSCKASGYAFSSSWMNWVRQAPGQGLEWIGRIYPGDGDTKYNGKFKGRAVLSADKSVSTAYLQISSLKAEDTAVYYCARRGTGGWYFDVWGQGTTVTVSS

SEQ ID NO: 3148 - protein, 119 aa, synthetic construct
QVQLVQSGPEVKKPGTSVKVSCKASGYAFSSSWMNWVRQARGQRLEWIGRIYPGDGDTKYNGKFKGRATLTADKSTSTAYMELSSLRSEDTAVYYCARRGTGGWYFDVWGQGTTVTVSS

SEQ ID NO: 3149 - protein, 119 aa, synthetic construct
EVQLVQSGAEVKKPGESLKISCKASGYAFSSSWMNWVRQMPGKGLEWIGRIYPGDGDTKYNGKFKGQATLSADKSISTAYLQWSSLKASDTAMYYCARRGTGGWYFDVWGQGTTVTVSS

SEQ ID NO: 3150 - protein, 119 aa, synthetic construct
QVQLVQSGSELKKPGASVKVSCKASGYAFSSSWMNWVRQAPGQGLEWIGRIYPGDGDTKYNGKFKGRAVLSADKSVSMAYLQISSLKAEDTAVYYCARRGTGGWYFDVWGQGTTVTVSS

SEQ ID NO: 3151 - protein, 119 aa, synthetic construct
QVQLVQSGHEVKQPGASVKVSCKASGYAFSSSWMNWVPQAPGQGLEWIGRIYPGDGDTKYNGKFKGRAVLSADKSASTAYLQISSLKAEDMAMYYCARRGTGGWYFDVWGQGTTVTVSS

SEQ ID NO: 3152 - protein, 119 aa, synthetic construct
EVQLVQSGAEVKKPGESLKISCKASGYAFSSSWMNWVRQMPGKGLEWIGRIYPGDGDTKYNGKFKGQATLSADKPISTAYLQWSSLKASDTAMYYCARRGTGGWYFDVWGQGTTVTVSS

SEQ ID NO: 3153 - protein, 119 aa, synthetic construct
EVQLVQSGAEVKKPGESLRISCKASGYAFSSSWMNWVRQMPGKGLEWIGRIYPGDGDTKYNGKFKGQATLSADKSISTAYLQWSSLKASDTAMYYCARRGTGGWYFDVWGQGTTVTVSS

SEQ ID NO: 3154 - protein, 119 aa, synthetic construct
EVQLVQSGAEVKKPGESLRISCKASGYAFSSSWMNWVRQMPGKGLEWIGRIYPGDGDTKYNGKFKGHATLSADKSISTAYLQWSSLKASDTAMYYCARRGTGGWYFDVWGQGTTVTVSS

SEQ ID NO: 3155 - protein, 119 aa, synthetic construct
QVQLVQSGAEVKKTGSSVKVSCKASGYAFSSSWMNWVRQAPRQALEWIGRIYPGDGDTKYNGKFKGRATLTADKSMSTAYMELSSLRSEDTAMYYCARRGTGGWYFDVWGQGTTVTVSS

SEQ ID NO: 3156 - protein, 119 aa, synthetic construct
EVQLVESGGGLVQPGRSLRLSCTASGYAFSSSWMNWVRQAPGKGLEWIGRIYPGDGDTKYNGKFKGRATLSADKSKSIAYLQMNSLKTEDTAVYYCARRGTGGWYFDVWGQGTTVTVSS

SEQ ID NO: 3157 - protein, 119 aa, synthetic construct
EVQLVESGGGLVQPGPSLRLSCTASGYAFSSSWMNWVRQAPGKGLEWIGRIYPGDGDTKYNGKFKGRATLSADKSKSIAYLQMNSLKTEDTAVYYCARRGTGGWYFDVWGQGTTVTVSS

SEQ ID NO: 3158 - protein, 119 aa, synthetic construct
QVQLQESGPGLVKPSQTLSLTCTASGYAFSSSWMNWVRQPPGKGLEWIGRIYPGDGDTKYNGKFKGRATLSADKSKSQASLKLSSVTAADTAVYYCARRGTGGWYFDVWGQGTTVTVSS

SEQ ID NO: 3159 - protein, 119 aa, synthetic construct
QVQLQESGPGLVKPSGTLSLTCAASGYAFSSSWMNWVRQPPGKGLEWIGRIYPGDGDTKYNGKFKGRATLSADKSKSQASLKLSSVTAADTAVYYCARRGTGGWYFDVWGQGTTVTVSS

SEQ ID NO: 3160 - protein, 119 aa, synthetic construct
EVQLVESGGGLVKPGRSLRLSCTASGYAFSSSWMNWVRQAPGKGLEWIGRIYPGDGDTKYNGKFKGRATLSADKSKSIAYLQMNSLKTEDTAVYYCARRGTGGWYFDVWGQGTTVTVSS

SEQ ID NO: 3161 - protein, 119 aa, synthetic construct
EVQLVESGGGLVQPGGSLKLSCAASGYAFSSSWMNWVRQASGKGLEWIGRIYPGDGDTKYNGKFKGRATLSADKSKSTAYLQMNSLKTEDTAVYYCARRGTGGWYFDVWGQGTTVTVSS

SEQ ID NO: 3162 - protein, 119 aa, synthetic construct
QVQLQESGPGLVKPSQTLSLTCAASGYAFSSSWMNWVRQPPGKGLEWIGRIYPGDGDTKYNGKFKGRATLSADKSKSQASLKLSSVTAADTAVYYCARRGTGGWYFDVWGQGTTVTVSS

SEQ ID NO: 3163 - protein, 119 aa, synthetic construct
EVQLVESGGGLVKPGGSLRLSCAASGYAFSSSWMNWVRQAPGKGLEWIGRIYPGDGDTKYNGKFKGRATLSADKSKSTAYLQMNSLKTEDTAVYYCARRGTGGWYFDVWGQGTTVTVSS

SEQ ID NO: 3164 - protein, 119 aa, synthetic construct
EVQLVESGGALVKPGGSLRLSCAASGYAFSSSWMNWVRQAPGKGLEWIGRIYPGDGDTKYNGKFKGRATLSADKSKSTAYLQMNSLKTEDTAVYYCARRGTGGWYFDVWGQGTTVTVSS

SEQ ID NO: 3165 - protein, 119 aa, synthetic construct
QVQLQESGPGLVKPSQTLSLTCAAYGYAFSSSWMNWVRQPPGKGLEWIGRIYPGDGDTKYNGKFKGRATLSADKSKSQASLKLSSVTAADTAVYYCARRGTGGWYFDVWGQGTTVTVSS

SEQ ID NO: 3166 - protein, 119 aa, synthetic construct
QVQLQESGSGLVKPSQTLSLTCAASGYAFSSSWMNWVRQPPGKGLEWIGRIYPGDGDTKYNGKFKGRATLSADKSKSQASLKLSSVTAADTAVYYCARRGTGGWYFDVWGQGTTVTVSS

SEQ ID NO: 3167 - protein, 119 aa, synthetic construct
EVQLVESGGGLVQPGGSLRLSCAASGYAFSSSWMNWVRQAPGKGLEWIGRIYPGDGDTKYNGKFKGRATLSADKSKSSAYLQMNSLKTEDTAVYYCARRGTGGWYFDVWGQGTTVTVSS

SEQ ID NO: 3168 - protein, 119 aa, synthetic construct
QVQLQESGPGLVKPSDTLSLTCTASGYAFSSSWMNWVRQPPGKGLEWIGRIYPGDGDTKYNGKFKGRATLSADKSKSQASLKLSSVTAADTAVYYCARRGTGGWYFDVWGQGTTVTVSS

SEQ ID NO: 3169 - protein, 119 aa, synthetic construct
QVQLQESGPGLVKPSQTLSLTCTASGYAFSSSWMNWVRQHPGKGLEWIGRIYPGDGDTKYNGKFKGRATLSADKSKSQASLKLSSVTAADTAVYYCARRGTGGWYFDVWGQGTTVTVSS

SEQ ID NO: 3170 - protein, 119 aa, synthetic construct
QVQLQESGPGLVKPSQTLSLTCTASGYAFSSSWMNWVRQHPGKGLEWIGRIYPGDGDTKYNGKFKGLATLSADKSKSQASLKLSSVTAADTAVYYCARRGTGGWYFDVWGQGTTVTVSS

SEQ ID NO: 3171 - protein, 119 aa, synthetic construct
QVQLVESGGGVVQPGRSLRLSCAASGYAFSSSWMNWVRQAPGKGLEWIGRIYPGDGDTKYNGKFKGRATLSADKSKSTAYLQMSSLRAEDTAVYYCARRGTGGWYFDVWGQGTTVTVSS

SEQ ID NO: 3172 - protein, 119 aa, synthetic construct
QVQLVESGGGLVKPGGSLRLSCAASGYAFSSSWMNWVRQAPGKGLEWIGRIYPGDGDTKYNGKFKGRATLSADKAKSSAYLQMNSLRAEDTAVYYCARRGTGGWYFDVWGQGTTVTVSS

SEQ ID NO: 3173 - protein, 119 aa, synthetic construct
QVQLVESGGGLVQPGGSLRLSCSASGYAFSSSWMNWVRQAPGKGLEWIGRIYPGDGDTKYNGKFKGRATLSADKSKSTAYLQMNSLRAEDTAVYYCARRGTGGWYFDVWGQGTTVTVSS

SEQ ID NO: 3174 - protein, 119 aa, synthetic construct
QVQLLESGGGLVKPGGSLRLSCAASGYAFSSSWMNWVRQAPGKGLEWIGRIYPGDGDTKYNGKFKGRATLSADKAKSSAYLQMNSLRAEDTAVYYCARRGTGGWYFDVWGQGTTVTVSS

SEQ ID NO: 3175 - protein, 119 aa, synthetic construct
EVQLVESGGGLVQPGGSLRLSCSASGYAFSSSWMNWVRQAPGKGLEWIGRIYPGDGDTKYNGKFKGRATLSADKSKSTAYLQMSSLRAEDTAVYYCARRGTGGWYFDVWGQGTTVTVSS

SEQ ID NO: 3176 - protein, 119 aa, synthetic construct
QVQLQESGPGLVKPSDTLSLTCAASGYAFSSSWMNWVRQPPGKGLEWIGRIYPGDGDTKYNGKFKGRATLSADKSKSQASLKLSSVTAVDTAVYYCARRGTGGWYFDVWGQGTTVTVSS

SEQ ID NO: 3177 - protein, 119 aa, synthetic construct
QVQLQESGPGLVKPSQTLSLTCAASGYAFSSSWMNWVRQPPGKGLEWIGRIYPGDGDTKYNGKFKGRATLSADKSKSQASLKLSSVTAVDTAVYYCARRGTGGWYFDVWGQGTTVTVSS

SEQ ID NO: 3178 - protein, 119 aa, synthetic construct
EVQLVESGGGLVQPGGSLRLSCSASGYAFSSSWMNWVRQAPGKGLEWIGRIYPGDGDTKYNGKFKGRATLSADKSKSTAYVQMSSLRAEDTAVYYCARRGTGGWYFDVWGQGTTVTVSS

SEQ ID NO: 3179 - protein, 119 aa, synthetic construct
QVQLVDSGGGVVQPGRSLRLSCAASGYAFSSSWMNWVRQAPGKGLEWIGRIYPGDGDTKYNGKFKGRATLSADKSKSTAYLQMNSLRAEDTAVYYCARRGTGGWYFDVWGQGTTVTVSS

SEQ ID NO: 3180 - protein, 119 aa, synthetic construct
QVQLVESGGGVVQPGRSLRLSCAASGYAFSSSWMNWVRQAPGKGLEWIGRIYPGDGDTKYNGKFKGRATLSADKSKSTAYLQMNSLRAEGTAVYYCARRGTGGWYFDVWGQGTTVTVSS

SEQ ID NO: 3181 - protein, 119 aa, synthetic construct
QVQLVESGGGVVQPGRSLRLSCAASGYAFSSSWMNWVRQAPGKGLEWIGRIYPGDGDTKYNGKFKGRATLSADKSKSTAYLQMNSLRAEDTAVYYCARRGTGGWYFDVWGQGTTVTVSS

SEQ ID NO: 3182 - protein, 119 aa, synthetic construct
EVQLVESGGGLVQPGGSLRLSCAASGYAFSSSWMNWVRQAPGKGLEWIGRIYPGDGDTKYNGKFKGRATLSADKSKSTAYLQMNSLRAEDTAVYYCARRGTGGWYFDVWGQGTTVTVSS

SEQ ID NO: 3183 - protein, 117 aa, synthetic construct
EVQLVESGGDLVKPGGSLKLSCAVSGFTFRSYGMSWVRQTPDKRLEWVALISSGGSYTYYTDSVKGRFTISRDNAKNTLYLQMSSLKSEDTAIYYCSRHGGNFFDYWGQGTTLTVSS

SEQ ID NO: 3184 - protein, 107 aa, synthetic construct
QIVLTQSPSIMSASPGEKVTMTCSVSSSVSYMHWYQQKSGTSPKRWIYDTSKLASGVPARFSGSGSGTSYSLTISSMEAEDAATYYCQQWSSNPQYTFGGGTKLEIK

SEQ ID NO: 3185 - protein, 107 aa, synthetic construct
DIVLTQSPAFLSVTPGEKVTITCSVSSSVSYMHWYQQKPDQAPKLLIYDTSKLASGVPSRFSGSGSGTDYTFTISSLEAEDAATYYCQQWSSNPQYTFGQGTKLEIK

SEQ ID NO: 3186 - protein, 107 aa, synthetic construct
AIQLTQSPSSLSASVGDRVTITCSVSSSVSYMHWYQQKPGKAPKLLIYDTSKLASGVPSRFSGSGSGTDYTLTISSLQPEDFATYYCQQWSSNPQYTFGQGTKLEIK

SEQ ID NO: 3187 - protein, 107 aa, synthetic construct
DIQLTQSPSFLSASVGDRVTITCSVSSSVSYMHWYQQKPGKAPKLLIYDTSKLASGVPSRFSGSGSGTEYTLTISSLQPEDFATYYCQQWSSNPQYTFGQGTKLEIK

SEQ ID NO: 3188 - protein, 107 aa, synthetic construct
DIQLTQSPSSLSASVGDRVTITCSVSSSVSYMHWYQQKPGKAPKLLIYDTSKLASGVPSRFSGSGSGTDYTLTISSLQPEDFATYYCQQWSSNPQYTFGQGTKLEIK

SEQ ID NO: 3189 - protein, 107 aa, synthetic construct
DIQLTQSPSSVSASVGDRVTITCSVSSSVSYMHWYQQKPGKAPKLLIYDTSKLASGVPSRFSGSGSGTDYTLTISSLQPEDFATYYCQQWSSNPQYTFGQGTKLEIK

SEQ ID NO: 3190 - protein, 107 aa, synthetic construct
DIQLTQSPSSLSASVGDRVTITCSVSSSVSYMHWYQQKPGKVPKLLIYDTSKLASGVPSRFSGSGSGTDYTLTISSLQPEDVATYYCQQWSSNPQYTFGQGTKLEIK

SEQ ID NO: 3191 - protein, 107 aa, synthetic construct
DIQLTQSPSSLSASVGDRVTITCSVSSSVSYMHWYQQKPGQAPKLLIYDTSKLASGVPSRFSGSGSGTDYTLTISSLQPEDVATYYCQQWSSNPQYTFGQGTKLEIK

SEQ ID NO: 3192 - protein, 107 aa, synthetic construct
EIVLTQSPDFQSVTPKEKVTITCSVSSSVSYMHWYQQKPDQSPKLLIYDTSKLASGVPSRFSGSGSGTDYTLTINSLEAEDAATYYCQQWSSNPQYTFGQGTKLEIK

SEQ ID NO: 3193 - protein, 107 aa, synthetic construct
AIRLTQSPFSLSASVGDRVTITCSVSSSVSYMHWYQQKPAKAPKLFIYDTSKLASGVPSRFSGSGSGTDYTLTISSLQPEDFATYYCQQWSSNPQYTFGQGTKLEIK

SEQ ID NO: 3194 - protein, 107 aa, synthetic construct
DIQLTQSPSSLSASVGDRVTITCSVSSSVSYMHWYQQKPGKAPKLLIYDTSKLASGVPSRFSGSGSGTDYTFTISSLQPEDIATYYCQQWSSNPQYTFGQGTKLEIK

SEQ ID NO: 3195 - protein, 107 aa, synthetic construct
EIVLTQSPATLSLSPGERATLSCSVSSSVSYMHWYQQKPGQAPKLLIYDTSKLASGIPARFSGSGSGTDYTLTISSLEPEDFAVYYCQQWSSNPQYTFGQGTKLEIK

SEQ ID NO: 3196 - protein, 107 aa, synthetic construct
DIQLTQSPSTLSASVGDRVTITCSVSSSVSYMHWYQQKPGKAPKLLIYDTSKLASGVPSRFSGSGSGTEYTLTISSLQPDDFATYYCQQWSSNPQYTFGQGTKLEIK

SEQ ID NO: 3197 - protein, 107 aa, synthetic construct
DIQLTQSPSSLSASVGDRVTITCSVSSSVSYMHWYQQKPGKTPKLLIYDTSKLASGIPSRFSGSGSGTDYTLTIRSLQPEDFATYYCQQWSSNPQYTFGQGTKLEIK

SEQ ID NO: 3198 - protein, 107 aa, synthetic construct
EIVLTQSPPTLSLSPGERVTLSCSVSSSVSYMHWYQQKPGQAPKLLIYDTSKLASGIPARFSGSGSGTDYTLTISSLQPEDFAVYYCQQWSSNPQYTFGQGTKLEIK

SEQ ID NO: 3199 - protein, 107 aa, synthetic construct
DIQLTQSPSSLSASVGDRVTITCSVSSSVSYMHWYQQKPGKAPKRLIYDTSKLASGVPSRFSGSGSGTEYTLTISSLQPEDFATYYCQQWSSNPQYTFGQGTKLEIK

SEQ ID NO: 3200 - protein, 107 aa, synthetic construct
EIVLTQSPATLSLSPGERATLSCSVSSSVSYMHWYQQKPGQAPKLLIYDTSKLASGIPARFSGSGPGTDYTLTISSLEPEDFAVYYCQQWSSNPQYTFGQGTKLEIK

SEQ ID NO: 3201 - protein, 107 aa, synthetic construct
EIVLTQSPATLSLSPGERATLSCSVSSSVSYMHWYQQKPGQAPKLLIYDTSKLASGIPARFSGSGSGTDYTLTISRLEPEDFAVYYCQQWSSNPQYTFGQGTKLEIK

SEQ ID NO: 3202 - protein, 107 aa, synthetic construct
EIVLTQSPATLSLSPGERATLSCSVSSSVSYMHWYQQKPGQAPKLLIYDTSKLASGIPARFSGSGSGTDYTLTISSLQPEDFAVYYCQQWSSNPQYTFGQGTKLEIK

SEQ ID NO: 3203 - protein, 107 aa, synthetic construct
EIVLTQSPATLSVSPGERATLSCSVSSSVSYMHWYQQKPGQAPKLLIYDTSKLASGIPARFSGSGSGTEYTLTISSLQSEDFAVYYCQQWSSNPQYTFGQGTKLEIK

SEQ ID NO: 3204 - protein, 107 aa, synthetic construct
EIVLTQSPATLSVSPGERATLSCSVSSSVSYMHWYQQKPGQAPKLLIYDTSKLASGIPARFSGSGSGTEYTLTISILQSEDFAVYYCQQWSSNPQYTFGQGTKLEIK

SEQ ID NO: 3205 - protein, 107 aa, synthetic construct
EIVLTQSPPTLSLSPGERVTLSCSVSSSVSYMHWYQQKPGQAPKLLIYDTSKLASSIPARFSGSGSGTDYTLTISSLQPEDFAVYYCQQWSSNPQYTFGQGTKLEIK

SEQ ID NO: 3206 - protein, 107 aa, synthetic construct
DIQLTQSPSSLSASVGDRVTITCSVSSSVSYMHWYQQKPGKAPKSLIYDTSKLASGVPSRFSGSGSGTDYTLTISSLQPEDFATYYCQQWSSNPQYTFGQGTKLEIK

SEQ ID NO: 3207 - protein, 107 aa, synthetic construct
DIQLTQSPSSLSASVGDRVTITCSVSSSVSYMHWYQQKPGKAPKRLIYDTSKLASGVPSRFSGSGSGTEYTLTISNLQPEDFATYYCQQWSSNPQYTFGQGTKLEIK

SEQ ID NO: 3208 - protein, 107 aa, synthetic construct
DIQLTQSPSAMSASVGDRVTITCSVSSSVSYMHWYQQKPGKVPKRLIYDTSKLASGVPSRFSGSGSGTEYTLTISSLQPEDFATYYCQQWSSNPQYTFGQGTKLEIK

SEQ ID NO: 3209 - protein, 107 aa, synthetic construct
EIVLTQSPATLSLSPGERATLSCSVSSSVSYMHWYQQKPGQAPKLLIYDTSKLASGIPDRFSGSGSGTDYTLTISRLEPEDFAVYYCQQWSSNPQYTFGQGTKLEIK

SEQ ID NO: 3210 - protein, 107 aa, synthetic construct
EIVLTQSPATLSLSPGERATLSCSVSSSVSYMHWYQQKPGLAPKLLIYDTSKLASGIPDRFSGSGSGTDYTLTISRLEPEDFAVYYCQQWSSNPQYTFGQGTKLEIK

SEQ ID NO: 3211 - protein, 107 aa, synthetic construct
EIVLTQSPGTLSLSPGERATLSCSVSSSVSYMHWYQQKPGQAPKLLIYDTSKLASGIPDRFSGSGSGTDYTLTISRLEPEDFAVYYCQQWSSNPQYTFGQGTKLEIK

SEQ ID NO: 3212 - protein, 107 aa, synthetic construct
DIQLTQSPSSLSASVGDRVTITCSVSSSVSYMHWYQQKPGKAPKSLIYDTSKLASGVPSKFSGSGSGTDYTLTISSLQPEDFATYYCQQWSSNPQYTFGQGTKLEIK

SEQ ID NO: 3213 - protein, 107 aa, synthetic construct
DIQLTQSPSSLSASVGDRVTITCSVSSSVSYMHWYQQKPEKAPKSLIYDTSKLASGVPSRFSGSGSGTDYTLTISSLQPEDFATYYCQQWSSNPQYTFGQGTKLEIK

SEQ ID NO: 3214 - protein, 107 aa, synthetic construct
DIVLTQSPDSLAVSLGERATINCSVSSSVSYMHWYQQKPGQPPKLLIYDTSKLASGVPDRFSGSGSGTDYTLTISSLQAEDVAVYYCQQWSSNPQYTFGQGTKLEIK

SEQ ID NO: 3215 - protein, 107 aa, synthetic construct
EIVLTQTPLSLSITPGEQASMSCSVSSSVSYMHWYLQKARPVPKLLIYDTSKLASGVPDRFSGSGSGTDYTLKISRVEAEDFGVYYCQQWSSNPQYTFGQGTKLEIK

SEQ ID NO: 3216 - protein, 107 aa, synthetic construct
EIVLTQTPLSLSITPGEQASISCSVSSSVSYMHWYLQKARPVPKLLIYDTSKLASGVPDRFSGSGSGTDYTLKISRVEAEDFGVYYCQQWSSNPQYTFGQGTKLEIK

SEQ ID NO: 3217 - protein, 107 aa, synthetic construct
DIVLTQSPLSLPVTPGEPASISCSVSSSVSYMHWYLQKPGQSPKLLIYDTSKLASGVPDRFSGSGSGTDYTLKISRVEAEDVGVYYCQQWSSNPQYTFGQGTKLEIK

SEQ ID NO: 3218 - protein, 107 aa, synthetic construct
DIVLTQSPLSLPVTLGQPASISCSVSSSVSYMHWYQQRPGQSPKRLIYDTSKLASGVPDRFSGSGSGTDYTLKISRVEAEDVGVYYCQQWSSNPQYTFGQGTKLEIK

SEQ ID NO: 3219 - protein, 107 aa, synthetic construct
DIVLTQTPLSLPVTPGEPASISCSVSSSVSYMHWYLQKPGQSPKLLIYDTSKLASGVPDRFSGSGSGTDYTLKISRVEAEDVGVYYCQQWSSNPQYTFGQGTKLEIK

SEQ ID NO: 3220 - protein, 107 aa, synthetic construct
DIVLTQTPLSLSVTPGQPASISCSVSSSVSYMHWYLQKPGQSPKLLIYDTSKLASGVPDRFSGSGSGTDYTLKISRVEAEDVGVYYCQQWSSNPQYTFGQGTKLEIK

SEQ ID NO: 3221 - protein, 107 aa, synthetic construct
DIVLTQTPLSLSVTPGQPASISCSVSSSVSYMHWYLQKPGQPPKLLIYDTSKLASGVPDRFSGSGSGTDYTLKISRVEAEDVGVYYCQQWSSNPQYTFGQGTKLEIK

SEQ ID NO: 3222 - protein, 107 aa, synthetic construct
DIQLIQSPSFLSASVGDRVSIICSVSSSVSYMHWYLQKPGKSPKLFIYDTSKLASGVSSRFSGRGSGTDYTLTIISLKPEDFAAYYCQQWSSNPQYTFGQGTKLEIK

SEQ ID NO: 3223 - protein, 107 aa, synthetic construct
DIVLTQTPLSSPVTLGQPASISCSVSSSVSYMHWYQQRPGQPPKLLIYDTSKLASGVPDRFSGSGAGTDYTLKISRVEAEDVGVYYCQQWSSNPQYTFGQGTKLEIK

SEQ ID NO: 3224 - protein, 107 aa, synthetic construct
EITLTQSPAFMSATPGDKVNISCSVSSSVSYMHWYQQKPGEAPKFIIYDTSKLASGIPPRFSGSGYGTDYTLTINNIESEDAAYYYCQQWSSNPQYTFGQGTKLEIK

SEQ ID NO: 3225 - protein, 117 aa, synthetic construct
EVQLVESGGGLVKPGGSLRLSCAVSGFTFRSYGMSWVRQAPGKGLEWVALISSGGSYTYYTDSVKGRFTISRDNSKNTLYLQMNSLKTEDTAVYYCSRHGGNFFDYWGQGTTVTVSS

SEQ ID NO: 3226 - protein, 117 aa, synthetic construct
EVQLVESGGALVKPGGSLRLSCAVSGFTFRSYGMSWVRQAPGKGLEWVALISSGGSYTYYTDSVKGRFTISRDNSKNTLYLQMNSLKTEDTAVYYCSRHGGNFFDYWGQGTTVTVSS

SEQ ID NO: 3227 - protein, 117 aa, synthetic construct
EVQLVESGGGLVQPGGSLRLSCAVSGFTFRSYGMSWVRQAPGKGLEWVALISSGGSYTYYTDSVKGRFTISRDNAKNTLYLQMNSLRAEDTAVYYCSRHGGNFFDYWGQGTTVTVSS

SEQ ID NO: 3228 - protein, 117 aa, synthetic construct
EVQLVESGGGLVQPGGSLRLSCAVSGFTFRSYGMSWVRQAPGKGLEWVALISSGGSYTYYTDSVKGRFTISRDNAKNSLYLQMNSLRAEDTAVYYCSRHGGNFFDYWGQGTTVTVSS

SEQ ID NO: 3229 - protein, 117 aa, synthetic construct
EVQLVESGGGLVQPGGSLRLSCAVSGFTFRSYGMSWVRQAPGKGLEWVALISSGGSYTYYTDSVKGRFTISRDNSKNSLYLQMNSLKTEDTAVYYCSRHGGNFFDYWGQGTTVTVSS

SEQ ID NO: 3230 - protein, 117 aa, synthetic construct
EVQLVESGGGLVQPGGSLRLSCAVSGFTFRSYGMSWVRQAPGKGLEWVALISSGGSYTYYTDSVKGRFTISRDNAKNSLYLQMNSLRAEDMAVYYCSRHGGNFFDYWGQGTTVTVSS

SEQ ID NO: 3231 - protein, 117 aa, synthetic construct
EVQLVESGGGLVQPGGSLRLSCAVSGFTFRSYGMSWVRQAPGKGLEWVALISSGGSYTYYTDSVKGQFTISRDNAKNTLYLQMNSLRAEDMAVYYCSRHGGNFFDYWGQGTTVTVSS

SEQ ID NO: 3232 - protein, 117 aa, synthetic construct
EVQLVESGGGLVKPGRSLRLSCTVSGFTFRSYGMSWVRQAPGKGLEWVALISSGGSYTYYTDSVKGRFTISRDNSKNILYLQMNSLKTEDTAVYYCSRHGGNFFDYWGQGTTVTVSS

SEQ ID NO: 3233 - protein, 117 aa, synthetic construct
EVQLVESGGGLVKPGGSLRLSCAVSGFTFRSYGMSWVRQAPGKGLEWVALISSGGSYTYYTDSVKGRFTISRDNAKNSLYLQMNSLRAEDTAVYYCSRHGGNFFDYWGQGTTVTVSS

SEQ ID NO: 3234 - protein, 117 aa, synthetic construct
EVQLVESGGGLVQPGGSLKLSCAVSGFTFRSYGMSWVRQASGKGLEWVALISSGGSYTYYTDSVKGRFTISRDNSKNTLYLQMNSLKTEDTAVYYCSRHGGNFFDYWGQGTTVTVSS

SEQ ID NO: 3235 - protein, 117 aa, synthetic construct
QVQLVESGGGVVQPGGSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3236 - protein, 117 aa, synthetic construct
QVQLVESGGGVVQPGRSLRLSCAVSGFTFRSYGMSWVRQAPGKGLEWVALISSGGSYTYYTDSVKGRFTISRDNSKNTLYLQMSSLRAEDTAVYYCSRHGGNFFDYWGQGTTVTVSS

SEQ ID NO: 3237 - protein, 117 aa, synthetic construct
EVQLVESGGGLVQPGGSLRLSCPVSGFTFRSYGMSWVRQAPGKGLEWVALISSGGSYTYYTDSVKGRFTISRDNANNSLYLQMNSLRAEDTAVYYCSRHGGNFFDYWGQGTTVTVSS

SEQ ID NO: 3238 - protein, 117 aa, synthetic construct
EVQLVESGGGLVQPGRSLRLSCTVSGFTFRSYGMSWVRQAPGKGLEWVALISSGGSYTYYTDSVKGRFTISRDNSKNILYLQMNSLKTEDTAVYYCSRHGGNFFDYWGQGTTVTVSS

SEQ ID NO: 3239 - protein, 117 aa, synthetic construct
EVQLVESGGGLVQPGPSLRLSCTVSGFTFRSYGMSWVRQAPGKGLEWVALISSGGSYTYYTDSVKGRFTISRDNSKNILYLQMNSLKTEDTAVYYCSRHGGNFFDYWGQGTTVTVSS

SEQ ID NO: 3240 - protein, 117 aa, synthetic construct
EVQLVESGGGLVQPGGSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3241 - protein, 117 aa, synthetic construct
EVQLVESGGGLVQPGGSLRLSCAVSGFTFRSYGMSWVRQAPGKGLEWVALISSGGSYTYYTDSVKGRFTISRDNAKNSLYLQMNSLRDEDTAVYYCSRHGGNFFDYWGQGTTVTVSS

SEQ ID NO: 3242 - protein, 117 aa, synthetic construct
QVQLVESGGGLVKPGGSLRLSCAVSGFTFRSYGMSWVRQAPGKGLEWVALISSGGSYTYYTDSVKGRFTISRDNAKNSLYLQMNSLRAEDTAVYYCSRHGGNFFDYWGQGTTVTVSS

SEQ ID NO: 3243 - protein, 117 aa, synthetic construct
QVQLVESGGGVVQPGRSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3244 - protein, 117 aa, synthetic construct
EVQLLESGGGLVQPGGSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3245 - protein, 117 aa, synthetic construct
EVQLVESGGGLVQPGGSLRLSCAVSGFTFRSYGMSWVRQAPGKGLEWVALISSGGSYTYYTDSVKGRFTISRHNSKNTLYLQMNSLRAEDTAVYYCSRHGGNFFDYWGQGTTVTVSS

SEQ ID NO: 3246 - protein, 117 aa, synthetic construct
EVQLVESGGGLIQPGGSLRLSCAVSGFTFRSYGMSWVRQPPGKGLEWVALISSGGSYTYYTDSVKGRFTISRDNSKNTLYLQMNSLRAEDTAVYYCSRHGGNFFDYWGQGTTVTVSS

SEQ ID NO: 3247 - protein, 117 aa, synthetic construct
EVQLVESGGGLIQPGGSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3248 - protein, 117 aa, synthetic construct
EVQLVESGGGLVQPGRSLRLSCAVSGFTFRSYGMSWVRQAPGKGLEWVALISSGGSYTYYTDSVKGRFTISRDNAKNSLYLQMNSLRAEDTALYYCSRHGGNFFDYWGQGTTVTVSS

SEQ ID NO: 3249 - protein, 117 aa, synthetic construct
QVQLVESGGGVVQPGRSLRLSCAVSGFTFRSYGMSWVRQAPGKGLEWVALISSGGSYTYYTDSVKGRFTISRDNSKNRLYLQMNSLRAEDTAVYYCSRHGGNFFDYWGQGTTVTVSS

SEQ ID NO: 3250 - protein, 117 aa, synthetic construct
QVQLVESGGGVVQPGRSLRLSCAVSGFTFRSYGMSWVRQAPGKGLEWVALISSGGSYTYYTDSVKGRFTISRDNSKNTLYLQMNSLRAEGTAVYYCSRHGGNFFDYWGQGTTVTVSS

SEQ ID NO: 3251 - protein, 117 aa, synthetic construct
QVQLVESGGGVVQPGRSLRLSCAVSGFTFRSYGMSWVRQAPGKGLEWVALISSGGSYTYYTDSVKGRFAISRDNSKNTLYLQMNSLRAEDTAVYYCSRHGGNFFDYWGQGTTVTVSS

SEQ ID NO: 3252 - protein, 117 aa, synthetic construct
QVQLVDSGGGVVQPGRSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3253 - protein, 117 aa, synthetic construct
EVQLVESGGGVVRPGGSLRLSCAVSGFTFRSYGMSWVRQAPGKGLEWVALISSGGSYTYYTDSVKGRFTISRDNAKNSLYLQMNSLRAEDTALYHCSRHGGNFFDYWGQGTTVTVSS

SEQ ID NO: 3254 - protein, 117 aa, synthetic construct
EVQLVESGGVVVQPGGSLRLSCAVSGFTFRSYGMSWVRQAPGKGLEWVALISSGGSYTYYTDSVKGRFTISRDNSKNSLYLQMNSLRAEDTALYYCSRHGGNFFDYWGQGTTVTVSS

SEQ ID NO: 3255 - protein, 117 aa, synthetic construct
EVQLVESGGGVVQPGGSLRLSCAVSGFTFRSYGMSWVRQAPGKGLEWVALISSGGSYTYYTDSVKGRFTISRDNSKNSLYLQMNSLRTEDTALYYCSRHGGNFFDYWGQGTTVTVSS

SEQ ID NO: 3256 - protein, 117 aa, synthetic construct
EVQLVESGGVVVQPGGSLRLSCAVSGFTFRSYGMSWVRQAPGKGLEWVALISSGGSYTYYTDSVKGRFTISRDNSKNSLYLQMNSLRTEDTALYYCSRHGGNFFDYWGQGTTVTVSS

SEQ ID NO: 3257 - protein, 117 aa, synthetic construct
EVQLVETGGGLIQPGGSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3258 - protein, 117 aa, synthetic construct
EVQLVESGGGLVQPGGSLRLSCAVSGFTFRSYGMSWVRQATGKGLEWVALISSGGSYTYYTDSVKGRFTISRENAKNSLYLQMNSLRAGDTAVYYCSRHGGNFFDYWGQGTTVTVSS

SEQ ID NO: 3259 - protein, 117 aa, synthetic construct
EVQLVESRGVLVQPGGSLRLSCAVSGFTFRSYGMSWVRQAPGKGLEWVALISSGGSYTYYTDSVKGRFTISRDNSKNTLHLQMNSLRAEDTAVYYCSRHGGNFFDYWGQGTTVTVSS

SEQ ID NO: 3260 - protein, 117 aa, synthetic construct
EVQLVESGGGLVQPGRSLRLSCAVSGFTFRSYGMSWVRQAPGKGLEWVALISSGGSYTYYTDSVKGRFTISRDNAKNSLYLQMNSLRAEDMALYYCSRHGGNFFDYWGQGTTVTVSS

SEQ ID NO: 3261 - protein, 117 aa, synthetic construct
QVQLVESGGGLVQPGGSLRLSCSVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3262 - protein, 117 aa, synthetic construct
EVQLVESGGGLVQPGGSLRLSCSVSGFTFRSYGMSWVRQAPGKGLEWVALISSGGSYTYYTDSVKGRFTISRDNSKNTLYLQMSSLRAEDTAVYYCSRHGGNFFDYWGQGTTVTVSS

SEQ ID NO: 3263 - protein, 117 aa, synthetic construct
QVQLVESGGGVVQPGRSLRLSCAVSGFTFRSYGMSWVRQAPGKGLEWVALISSGGSYTYYTDSVKGRFTISRDNSTNTLFLQMNSLRAEDTAVYYCSRHGGNFFDYWGQGTTVTVSS

SEQ ID NO: 3264 - protein, 117 aa, synthetic construct
QVQLLESGGGLVKPGGSLRLSCAVSGFTFRSYGMSWVRQAPGKGLEWVALISSGGSYTYYTDSVKGRFTISRDNAKNSLYLQMNSLRAEDTAVYYCSRHGGNFFDYWGQGTTVTVSS

SEQ ID NO: 3265 - protein, 117 aa, synthetic construct
EVQLVESGEGLVQPGGSLRLSCAVSGFTFRSYGMSWVRQAPGKGLEWVALISSGGSYTYYTDSVKGRFTISRDNSKNTLYLQMGSLRAEDMAVYYCSRHGGNFFDYWGQGTTVTVSS

SEQ ID NO: 3266 - protein, 117 aa, synthetic construct
EVQLVESGGGLVQPGGSLRLSCAVSGFTFRSYGMSWVRQAPGKGLEWVALISSGGSYTYYTDSVKGRFTISRDNSKNTLYLQMGSLRAEDMAVYYCSRHGGNFFDYWGQGTTVTVSS

SEQ ID NO: 3267 - protein, 117 aa, synthetic construct
EVQLVESGGGLVQPGGSLRLSCSVSGFTFRSYGMSWVRQAPGKGLEWVALISSGGSYTYYTDSVKGRFTISRDNSKNTLYVQMSSLRAEDTAVYYCSRHGGNFFDYWGQGTTVTVSS

SEQ ID NO: 3268 - protein, 117 aa, synthetic construct
EVQLVESGGGLVQPGGSLRLSCAVSGFTFRSYGMSWVRQAPGKGLEWVALISSGGSYTYYTDSVKGRFIISRDNSRNSLYLQKNRRRAEDMAVYYCSRHGGNFFDYWGQGTTVTVSS

SEQ ID NO: 3269 - protein, 117 aa, synthetic construct
EVQLVESGGGLVQPGGSLRLSCAVSGFTFRSYGMSWVHQAPGKGLEWVALISSGGSYTYYTDSVKGRFIISRDNSRNTLYLQTNSLRAEDTAVYYCSRHGGNFFDYWGQGTTVTVSS

SEQ ID NO: 3270 - protein, 117 aa, synthetic construct
EVHLVESGGGLVQPGGALRLSCAVSGFTFRSYGMSWVRQATGKGLEWVALISSGGSYTYYTDSVKGRFTISRENAKNSLYLQMNSLRAGDTAVYYCSRHGGNFFDYWGQGTTVTVSS

SEQ ID NO: 3271 - protein, 117 aa, synthetic construct
EVQLVESGGGLVQPRGSLRLSCAVSGFTFRSYGMSWVRQAPGKGLEWVALISSGGSYTYYTDSVKGRFTISRDNSKNTLYLQMNNLRAEGTAVYYCSRHGGNFFDYWGQGTTVTVSS

SEQ ID NO: 3272 - protein, 117 aa, synthetic construct
EVQLVESGGGLVQPRGSLRLSCAVSGFTFRSYGMSWVRQAPGKGLEWVALISSGGSYTYYTDSVKGRFTISRDNSKNTLYLQMNNLRAEGTAAYYCSRHGGNFFDYWGQGTTVTVSS

SEQ ID NO: 3273 - protein, 117 aa, synthetic construct
QVQLVQSGAEVKKPGASVKVSCKVSGFTFRSYGMSWVRQAPGKGLEWVALISSGGSYTYYTDSVKGRFTITRDNSTNTLYMELSSLRSEDTAVYYCSRHGGNFFDYWGQGTTVTVSS

SEQ ID NO: 3274 - protein, 117 aa, synthetic construct
QVQLVQSGSELKKPGASVKVSCKVSGFTFRSYGMSWVRQAPGQGLEWVALISSGGSYTYYTDSVKGRFVISRDNSVNTLYLQISSLKAEDTAVYYCSRHGGNFFDYWGQGTTVTVSS

SEQ ID NO: 3275 - protein, 470 aa, synthetic construct
METDTLLLWVLLLWVPGSTGQVQLVESGGGVVQPGRSLRLSCAASGIDFSRYWMSWVRQAPGKGLEWVGEINPDSSTINYAPSLKDRFTISRDNSKNTLYLQMSSLRAEDTAVYYCASLYYDYGDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3276 - protein, 503 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIKGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3277 - protein, 234 aa, synthetic construct; identical to SEQ ID NO: 1007

SEQ ID NO: 3278 - protein, 469 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3279 - protein, 234 aa, synthetic construct
METDTLLLWVLLLWVPGSTGDIQMTQSPSSLSASVGDRVTITCKASQNVGINVVWHQQKPGKVPKALIYSSSHRYSGVPSRFSGSGSGTDFTLTISSLQPEDVATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3280 - protein, 247 aa, synthetic construct
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 3281 - protein, 520 aa, synthetic construct
METDTLLLWVLLLWVPGSTGEVQLVESGGGLVQPGGSLRLSCAASGFTFSNFGMHWVRQAPGKGLEWVSYISSGSSTIYYADTLKGRFTISRDNSKNTLYLQMNSLRAEDTAVYYCARRGEGAMDYWGQGTTVTVSSGGGGSGGGGSGGGGSGGGGSDNQLTQSPSFLSASVGDRVTITCRASSSVNYIYWYQQKPGKAPKLLIYYTSNLAPGVPSRFSGSGSGNEYTLTISSLQPEDFATYYCQQFTSSPFTFGQGTKLEIKGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGLNDIFEAQKIEWHE

SEQ ID NO: 3282-3283 - skipped (2 entries)

SEQ ID NO: 3284 - protein, 469 aa, synthetic construct
METDTLLLWVLLLWVPGSTGQVQLQQSGPELVKPGASVKISCKASGYAFSSSWMNWVKQRPGQGLEWIGRIYPGDGDTKYNGKFKGKATLTADKSSSTAYMHLSSLTSVDSAVYFCARRGTGGWYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3285 - protein, 238 aa, synthetic construct
METDTLLLWVLLLWVPGSTGDIVLTQSPASLAVSLGQRATISCRASESVDSSGNSFMHWYQQKPGQPPQLLIYRASNLESGIPARFSGSGSRTDFTLTINPVEADDVATFYCQQSFDDPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3286 - protein, 739 aa, synthetic construct
METDTLLLWVLLLWVPGSTGQVQLVESGGGVVQPGRSLRLSCAASGIDFSRYWMSWVRQAPGKGLEWVGEINPDSSTINYAPSLKDRFTISRDNSKNTLYLQMSSLRAEDTAVYYCASLYYDYGDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSTYAMNWVRQAPGKGLEWVSRIRSKYNNYATYYADSVKDRFTISRDDSKNTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGGKAALTLSGAQPEDEAEYYCALWYSNLWVFGGGTKLTVL

SEQ ID NO: 3287 - protein, 247 aa, synthetic construct
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3288 - skipped

SEQ ID NO: 3289 - protein, 107 aa, synthetic construct; identical to SEQ ID NO: 230
  VARIANT 30: note = G, E, A, or D
  VARIANT 31: note = N or D
  VARIANT 32: note = R or K
  VARIANT 36: note = Y or H
  VARIANT 56: note = K or S

SEQ ID NO: 3290 - protein, 119 aa, synthetic construct; identical to SEQ ID NO: 231
  VARIANT 27: note = H, T, G or Y
  VARIANT 28: note = D, T or S
  VARIANT 30: note = H, R, D, K or T
  VARIANT 31: note = L, D, K, T or N
  VARIANT 32: note = W, F, T, I, Y or G
  VARIANT 50: note = R or W
  VARIANT 51: note = V, I or F
  VARIANT 52: note = F, S or Y
  VARIANT 53: note = A or P
  VARIANT 57: note = N or S
  VARIANT 58: note = T, V, Y or I
  VARIANT 59: note = K or R
  VARIANT 98: note = G or V
  VARIANT 100: note = Y or I
  VARIANT 103: note = Y or A
  VARIANT 104: note = D or G

SEQ ID NO: 3291 - protein, 238 aa, Homo sapiens
DLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFS

SEQ ID NO: 3292 - protein, 284 aa, Homo sapiens
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHW

SEQ ID NO: 3293 - protein, 287 aa, Homo sapiens
AEPHSLRYNLMVLSQDESVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKVLVLQSQRTD

SEQ ID NO: 3294 - protein, 191 aa, Homo sapiens
GWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPG

SEQ ID NO: 3295 - protein, 329 aa, Homo sapiens
QDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGG

SEQ ID NO: 3296 - protein, 323 aa, Homo sapiens
WPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRN

SEQ ID NO: 3297 - protein, 330 aa, Homo sapiens
QNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDSRAGEEGSIRAVDH

SEQ ID NO: 3298 - protein, 155 aa, Homo sapiens
QRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 3299 - protein, 294 aa, Homo sapiens
WTYHYSEKPMNWQRARRFCRDNYTDLVAIQNKAEIEYLEKTLPFSRSYYWIGIRKIGGIWTWVGTNKSLTEEAENWGDGEPNNKKNKEDCVEIYIKRNKDAGKWNDDACHKLKAALCYTASCQPWSCSGHGECVEIINNYTCNCDVGYYGPQCQFVIQCEPLEAPELGTMDCTHPLGNFSFSSQCAFSCSEGTNLTGIEETTCGPFGNWSSPEPTCQVIQCEPLSAPDLGIMNCSHPLASFSFTSACTFICSEGTELIGKKKTICESSGIWSNPSPICQKLDKSFSMIKEGDYN

SEQ ID NO: 3300 - protein, 243 aa, Homo sapiens
RYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 3301 - protein, 124 aa, Homo sapiens
KLTRDSQSLCPYDWIGFQNKCYYFSKEEGDWNSSKYNCSTQHADLTIIDNIEEMNFLRRYKCSSDHWIGLKMAKNRTGQWVDGATFTKSFGMRGSEGCAYLSDDGAATARCYTERKWICRKRIH

SEQ ID NO: 3302 - protein, 194 aa, Homo sapiens
QGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARS

SEQ ID NO: 3303 - protein, 133 aa, Homo sapiens
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 3304 - protein, 149 aa, Homo sapiens
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 3305 - protein, 205 aa, Homo sapiens
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 3306 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 3307 - protein, 5 aa, synthetic construct
GGGGS

SEQ ID NO: 3308 - protein, 10 aa, synthetic construct
GGGGSGGGGS

SEQ ID NO: 3309 - protein, 15 aa, synthetic construct
GGGGSGGGGSGGGGS

SEQ ID NO: 3310 - protein, 17 aa, synthetic construct
DVPSGPGGGGGSGGGGS

SEQ ID NO: 3311 - protein, 455 aa, synthetic construct; identical to SEQ ID NO: 3306

SEQ ID NO: 3312 - protein, 413 aa, synthetic construct
FRGPLLPNRPFTTVWNANTQWCLERHGVDVDVSVFDVVANPGQTFRGPDMTIFYSSQGTYPYYTPTGEPVFGGLPQNASLIAHLARTFQDILAAIPAPDFSGLAVIDWEAWRPRWAFNWDTKDIYRQRSRALVQAQHPDWPAPQVEAVAQDQFQGAARAWMAGTLQLGRALRPRGLWGFYGFPDCYNYDFLSPNYTGQCPSGIRAQNDQLGWLWGQSRALYPSIYMPAVLEGTGKSQMYVQHRVAEAFRVAVAAGDPNLPVLPYVQIFYDTTNHFLPLDELEHSLGESAAQGAAGVVLWVSWENTRTKESCQAIKEYMDTTLGPFILNVTSGALLCSQALCSGHGRCVRRTSHPKALLLLNPASFSIQLTPGGGPLSLRGALSLEDQAQMAVEFKCRCYPGWQAPWCERKSMW

SEQ ID NO: 3313 - protein, 551 aa, synthetic construct
YNFFPRKPKWDKNQITYRIIGYTPDLDPETVDDAFARAFQVWSDVTPLRFSRIHDGEADIMINFGRWEHGDGYPFDGKDGLLAHAFAPGTGVGGDSHFDDDELWTLGEGQVVRVKYGNADGEYCKFPFLFNGKEYNSCTDTGRSDGFLWCSTTYNFEKDGKYGFCPHEALFTMGGNAEGQPCKFPFRFQGTSYDSCTTEGRTDGYRWCGTTEDYDRDKKYGFCPETAMSTVGGNSEGAPCVFPFTFLGNKYESCTSAGRSDGKMWCATTANYDDDRKWGFCPDQGYSLFLVAAHEFGHAMGLEHSQDPGALMAPIYTYTKNFRLSQDDIKGIQELYGASPDIDLGTGPTPTLGPVTPEICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 3314 - protein, 14 aa, synthetic construct
AEAAAKEAAAKAAA

SEQ ID NO: 3315 - protein, 19 aa, synthetic construct
AEAAAKEAAAKEAAAKAAA

SEQ ID NO: 3316 - protein, 24 aa, synthetic construct
AEAAAKEAAAKEAAAKEAAAKAAA

SEQ ID NO: 3317 - protein, 29 aa, synthetic construct
AEAAAKEAAAKEAAAKEAAAKEAAAKAAA

SEQ ID NO: 3318-3399 - skipped (82 entries)

SEQ ID NO: 3400 - protein, 119 aa, synthetic construct
QVQLQQPGTELVKPGASVKLSCKASGYTFTTYWMHWVKQRPGHGLEWIGNFNPNNGDTNYNEKFKTKATLTVDKSSSTAYMQLSSLTSEDSAVYYCARDDYGRYYFDYWGQGTTLTVSS

SEQ ID NO: 3401 - protein, 112 aa, synthetic construct
DIVMSQSPSSLAVSAGEKVTMSCKSSQSLLNSRTRKNYLAWYQQKPGQAPKLLIYWAFTRESGVPDRFTGSGSGTDFTLTISSVQAEDLAIYYCKQSFILRTFGGGTKLEIK

SEQ ID NO: 3402-3436 - skipped (35 entries)

SEQ ID NO: 3437 - protein, 27 aa, synthetic construct
  VARIANT 2..26: note = This region may encompass 2-5 repeating EAAAK units
AEAAAKEAAAKEAAAKEAAAKEAAAKA

SEQ ID NO: 3438 - protein, 117 aa, synthetic construct; identical to SEQ ID NO: 24

SEQ ID NO: 3439 - protein, 116 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSS

SEQ ID NO: 3440 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGYPPDFTFGQGTKVEIK

SEQ ID NO: 3441 - protein, 119 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVAPYFAPFDYWGQGTLVTVSS

SEQ ID NO: 3442 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGNPPLYTFGQGTKVEIK

SEQ ID NO: 3443 - protein, 116 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARNGYLGDYWGQGTLVTVSS

SEQ ID NO: 3444 - protein, 111 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQAMQIPTFGQGTKVEIK

SEQ ID NO: 3445 - protein, 120 aa, synthetic construct
QVQLQQSGGGLVQPGGSLKLSCAASGIDFSRYWMSWVRRAPGKGLEWIGEINPDSSTINYAPSLKDKFIISRDNAKNTLYLQMSKVRSEDTALYYCASLYYDYGDAMDYWGQGTSVTVSS

SEQ ID NO: 3446 - protein, 108 aa, synthetic construct
DIVMTQSQRFMTTSVGDRVSVTCKASQSVDSNVAWYQQKPRQSPKALIFSASLRFSGVPARFTGSGSGTDFTLTISNLQSEDLAEYFCQQYNNYPLTFGAGTKLELKR

SEQ ID NO: 3447 - protein, 123 aa, synthetic construct
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSS

SEQ ID NO: 3448 - protein, 111 aa, synthetic construct
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLG

SEQ ID NO: 3449 - protein, 101 aa, synthetic construct
IAGITQAPTSQILAAGRRMTLRCTQDMRHNAMYWYRQDLGLGLRLIHYSNTAGTTGKGEVPDGYSVSRANTDDFPLTLASAVPSQTSVYFCASSWSFGTEA

SEQ ID NO: 3450 - protein, 100 aa, synthetic construct
GVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGEL

SEQ ID NO: 3451 - protein, 100 aa, synthetic construct
KVTQSSRYLVKRTGEKVFLECVQDMDHENMFWYRQDPGLGLRLIYFSYDVKMKEKGDIPEGYSVSREKKERFSLILASASTDQTSMYLCASSFLGTGVEQ

SEQ ID NO: 3452 - protein, 100 aa, synthetic construct
GGITQSPKYLFRKEGQNVTLSCEQNLNHDAMYWYRQDPGQGLRLIYYSQIVNDFQKGDIAEGYSVSREKKESFPLTVTSAQKNPTAFYLCASSSRSSYEQ

SEQ ID NO: 3453 - protein, 101 aa, synthetic construct
GVTQTPKHLITATGQRVTLRCSPRSGDLSVYWYQQSLDQGLQFLIQYYNGEERAKGNILERFSAQQFPDLHSELNLSSLELGDSALYFCASSVAGTPSYEQ

SEQ ID NO: 3454 - protein, 100 aa, synthetic construct
AGVTQTPRYLIKTRGQQVTLSCSPISGHRSVSWYQQTPGQGLQFLFEYFSETQRNKGNFPGRFSGRQFSNSRSEMNVSTLELGDSALYLCASSFNMATGQ

SEQ ID NO: 3455 - protein, 99 aa, synthetic construct
AVVSQHPSRVIVKSGTSVKIECRSLDFQATTMFWYRQFPSLMLMATSNEGSKATYEQGVEKDKFLINHASLTLSTLTVTSAHPEDSGFYICSALAGDTQ

SEQ ID NO: 3456 - protein, 106 aa, synthetic construct
MDAGVIQSPRHEVTEMGQQVTLRCKPISGHDYLFWYRQTMMRGLELLIYFNNNVPIDDSGMPEDRFSAKMPNASFSTLKIQPSEPRDSAVYFCASSLWEKLAKNIQ

SEQ ID NO: 3457 - protein, 94 aa, synthetic construct
DTGITQTPKYLVTAMGSKRTMKREHLGHDSMYWYRQKAKKSLEFMFYYNCKEFIENKTVPNHFTPECPDSSRLYLHVVALQQEDSAAYLCTSSQ

SEQ ID NO: 3458 - protein, 96 aa, synthetic construct
EPEVTQTPSHQVTQMGQEVILRCVPISNHLYFYWYRQILGQKVEFLVSFYNNEISEKSEIFDDQFSVERPDGSNFTLKIRSTKLEDSAMYFCASSE

SEQ ID NO: 3459 - protein, 95 aa, synthetic construct
DTAVSQTPKYLVTQMGNDKSIKCEQNLGHDTMYWYKQDSKKFLKIMFSYNNKELIINETVPNRFSPKSPDKAHLNLHINSLELGDSAVYFCASSQ

SEQ ID NO: 3460 - protein, 94 aa, synthetic construct
DTAVSQTPKYLVTQMGKKESLKEQNLGHNAMYWYKQDSKKFLKTMFIYSNKEPILNETVPNRFSPDSPDKAHLNLHINSLELGDSAVYFCASSQ

SEQ ID NO: 3461 - protein, 95 aa, synthetic construct
DTEVTQTPKHLVMGMTNKKSLKCEQHMGHRAMYWYKQKAKKPPELMFVYSYEKLSINESVPSRFSPECPNSSLLNLHLHALQPEDSALYLCASSQ

SEQ ID NO: 3462 - protein, 95 aa, synthetic construct
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSQ

SEQ ID NO: 3463 - protein, 95 aa, synthetic construct
ETGVTQTPRHLVMGMTNKKSLKCEQHLGHNAMYWYKQSAKKPLELMFVYSLEERVENNSVPSRFSPECPNSSHLFLHLHTLQPEDSALYLCASSQ

SEQ ID NO: 3464 - protein, 95 aa, synthetic construct
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSL

SEQ ID NO: 3465 - protein, 95 aa, synthetic construct
EAGVTQSPTHLIKTRGQQVTLRCSPISGHSSVSWYQQAPGQGPQFIFEYANELRRSEGNFPNRFSGRQFHDCCSEMNVSALELGDSALYLCARSL

SEQ ID NO: 3466 - protein, 95 aa, synthetic construct
ETGVTQSPTHLIKTRGQQVTLRCSSQSGHNTVSWYQQALGQGPQFIFQYYREEENGRGNFPPRFSGLQFPNYSSELNVNALELDDSALYLCASSL

SEQ ID NO: 3467 - protein, 95 aa, synthetic construct
DAGVTQSPTHLIKTRGQQVTLRCSPISGHKSVSWYQQVLGQGPQFIFQYYEKEERGRGNFPDRFSARQFPNYSSELNVNALLLGDSALYLCASSL

SEQ ID NO: 3468 - protein, 95 aa, synthetic construct
DAGVTQSPTHLIKTRGQQVTLRCSPKSGHDTVSWYQQALGQGPQFIFQYYEEEERQRGNFPDRFSGHQFPNYSSELNVNALLLGDSALYLCASSL

SEQ ID NO: 3469 - protein, 95 aa, synthetic construct
DAGVTQSPTHLIKTRGQHVTLRCSPISGHTSVSSYQQALGQGPQFIFQYYEKEERGRGNFPDQFSGHQFPNYSSELNVNALLLGDSALYLCASSL

SEQ ID NO: 3470 - protein, 95 aa, synthetic construct
EAGVTQSPTHLIKTRGQQATLRCSPISGHTSVYWYQQALGLGLQFLLWYDEGEERNRGNFPPRFSGRQFPNYSSELNVNALELEDSALYLCASSL

SEQ ID NO: 3471 - protein, 95 aa, synthetic construct
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSE

SEQ ID NO: 3472 - protein, 95 aa, synthetic construct
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSY

SEQ ID NO: 3473 - protein, 95 aa, synthetic construct; identical to SEQ ID NO: 3472

SEQ ID NO: 3474 - protein, 95 aa, synthetic construct
IAGITQAPTSQILAAGRRMTLRCTQDMRHNAMYWYRQDLGLGLRLIHYSNTAGTTGKGEVPDGYSVSRANTDDFPLTLASAVPSQTSVYFCASSD

SEQ ID NO: 3475 - protein, 95 aa, synthetic construct
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSY

SEQ ID NO: 3476 - protein, 95 aa, synthetic construct
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASSY

SEQ ID NO: 3477 - protein, 95 aa, synthetic construct
NAGVTQTPKFHVLKTGQSMTLLCAQDMNHEYMYRYRQDPGKGLRLIYYSVAAALTDKGEVPNGYNVSRSNTEDFPLKLESAAPSQTSVYFCASSY

SEQ ID NO: 3478 - protein, 94 aa, synthetic construct
NAGVTQTPKFHILKTGQSMTLQCAQDMNHGYMSWYRQDPGMGLRLIYYSAAAGTTDKEVPNGYNVSRLNTEDFPLRLVSAAPSQTSVYLCASSY

SEQ ID NO: 3479 - protein, 95 aa, synthetic construct
NAGVTQTPKFHILKTGQSMTLQCAQDMNHGYLSWYRQDPGMGLRRIHYSVAAGITDKGEVPDGYNVSRSNTEDFPLRLESAAPSQTSVYFCASSY

SEQ ID NO: 3480 - protein, 96 aa, synthetic construct
GAGVSQSLRHKVAKKGKDVALRYDPISGHNALYWYRQSLGQGLEFPIYFQGKDAADKSGLPRDRFSAQRSEGSISTLKFQRTQQGDLAVYLCASSS

SEQ ID NO: 3481 - protein, 96 aa, synthetic construct
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSL

SEQ ID NO: 3482 - protein, 96 aa, synthetic construct
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSL

SEQ ID NO: 3483 - protein, 96 aa, synthetic construct
GAGVSQSPRYKVAKRGRDVALRCDSISGHVTLYWYRQTLGQGSEVLTYSQSDAQRDKSGRPSGRFSAERPERSVSTLKIQRTEQGDSAVYLCASSL

SEQ ID NO: 3484 - protein, 96 aa, synthetic construct
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSL

SEQ ID NO: 3485 - protein, 96 aa, synthetic construct
GAGVSQSPRYKVTKRGQDVTLRCDPISSHATLYWYQQALGQGPEFLTYFNYEAQPDKSGLPSDRFSAERPEGSISTLTIQRTEQRDSAMYRCASSL

SEQ ID NO: 3486 - protein, 96 aa, synthetic construct
GAGVSQSPRYKVAKRGQDVALRCDPISGHVSLFWYQQALGQGPEFLTYFQNEAQLDKSGLPSDRFFAERPEGSVSTLKIQRTQQEDSAVYLCASSL

SEQ ID NO: 3487 - protein, 96 aa, synthetic construct
DTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSL

SEQ ID NO: 3488 - protein, 95 aa, synthetic construct
DSGVTQTPKHLITATGQRVTLRCSPRSGDLSVYWYQQSLDQGLQFLIQYYNGEERAKGNILERFSAQQFPDLHSELNLSSLELGDSALYFCASSV

SEQ ID NO: 3489 - protein, 95 aa, synthetic construct
DAEITQSPRHKITETGRQVTLACHQTWNHNNMFWYRQDLGHGLRLIHYSYGVQDTNKGEVSDGYSVSRSNTEDLPLTLESAASSQTSVYFCASSE

SEQ ID NO: 3490 - protein, 95 aa, synthetic construct
DAGITQSPRYKITETGRQVTLMCHQTWSHSYMFWYRQDLGHGLRLIYYSAAADITDKGEVPDGYVVSRSKTENFPLTLESATRSQTSVYFCASSE

SEQ ID NO: 3491 - protein, 95 aa, synthetic construct
DAGITQSPRHKVTETGTPVTLRCHQTENHRYMYWYRQDPGHGLRLIHYSYGVKDTDKGEVSDGYSVSRSKTEDFLLTLESATSSQTSVYFCAISE

SEQ ID NO: 3492 - protein, 96 aa, synthetic construct
EAEVAQSPRYKITEKSQAVAFWCDPISGHATLYWYRQILGQGPELLVQFQDESVVDDSQLPKDRFSAERLKGVDSTLKIQPAELGDSAMYLCASSL

SEQ ID NO: 3493 - protein, 96 aa, synthetic construct
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASSL

SEQ ID NO: 3494 - protein, 96 aa, synthetic construct
EAGVVQSPRYKIIEKKQPVAFWCNPISGHNTLYWYLQNLGQGPELLIRYENEEAVDDSQLPKDRFSAERLKGVDSTLKIQPAELGDSAVYLCASSL

SEQ ID NO: 3495 - protein, 95 aa, synthetic construct
DAGVIQSPRHKVTEMGQSVTLRCEPISGHNDLLWYRQTFVQGLELLNYFCSWTLVDDSGVSKDFSAQMPDVSFSTLRIQPMEPRDLGLYFCASSF

SEQ ID NO: 3496 - protein, 95 aa, synthetic construct
DAGIIQSPKHEVTEMGQTVTLRCEPIFGHNFLFWYRDTFVQGLELLSYFRSSIIDNAGMPTERFSAERPDGSFSTLKIQPAEQGDSAVYVCASRL

SEQ ID NO: 3497 - protein, 96 aa, synthetic construct
DAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSL

SEQ ID NO: 3498 - protein, 96 aa, synthetic construct
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSL

SEQ ID NO: 3499 - protein, 96 aa, synthetic construct
DARVTQTPRHKVTEMGQEVTMRCQPILGHNTVFWYRQTMMQGLELLAYFRNRAPLDDSGMPKDRFSAEMPDATLATLKIQPSEPRDSAVYFCASGL

SEQ ID NO: 3500 - protein, 95 aa, synthetic construct
AAGVIQSPRHLIKEKRETATLKCYPIPRHDTVYWYQQGPGQDPQFLISFYEKMQSDKGSIPDRFSAQQFSDYHSELNMSSLELGDSALYFCASSL

SEQ ID NO: 3501 - protein, 96 aa, synthetic construct
EAGVTQFPSHSVIEKGQTVTLRCDPISGHDNLYWYRRVMGKEIKFLLHFVKESKQDESGMPNNRFLAERTGGTYSTLKVQPAELEDSGVYFCASSQ

SEQ ID NO: 3502 - protein, 95 aa, synthetic construct
DAMVIQNPRYQVTQFGKPVTLSCSQTLNHNVMYWYQQKSSQAPKLLFHYYDKDFNNEADTPDNFQSRRPNTSFCFLDIRSPGLGDTAMYLCATSR

SEQ ID NO: 3503 - protein, 96 aa, synthetic construct
GEEVAQTPKHLVRGEGQKAKLYCAPIKGHSYVFWYQQVLKNEFKFLISFQNENVFDETGMPKERFSAKCLPNSPCSLEIQATKLEDSAVYFCASSQ

SEQ ID NO: 3504 - protein, 95 aa, synthetic construct
EPGVSQTPRHKVTNMGQEVILRCDPSSGHMFVHWYRQNLRQEMKLLISFQYQNIAVDSGMPKERFTAERPNGTSSTLKIHPAEPRDSAVYLYSSG

SEQ ID NO: 3505 - protein, 96 aa, synthetic construct
NAGVMQNPRHLVRRRGQEARLRCSPMKGHSHVYWYRQLPEEGLKFMVYLQKENIIDESGMPKERFSAEFPKEGPSILRIQQVVRGDSAAYFCASSP

SEQ ID NO: 3506 - protein, 95 aa, synthetic construct
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSI

SEQ ID NO: 3507 - protein, 97 aa, synthetic construct
GAVVSQHPSWVICKSGTSVKIECRSLDFQATTMFWYRQFPKQSLMLMATSNEGSKATYEQGVEKDKFLINHASLTLSTLTVTSAHPEDSSFYICSAR

SEQ ID NO: 3508 - protein, 96 aa, synthetic construct
DTKVTQRPRLLVKASEQKAKMDCVPIKAHSYVYWYRKKLEEELKFLVYFQNEELIQKAEIINERFLAQCSKNSSCTLEIQSTESGDTALYFCASSK

SEQ ID NO: 3509 - protein, 96 aa, synthetic construct
HAKVTQTPGHLVKGKGQKTKMDCTPEKGHTFVYWYQQNQNKEFMLLISFQNEQVLQETEMHKKRFSSQCPKNAPCSLAILSSEPGDTALYLCASSQ

SEQ ID NO: 3510 - protein, 96 aa, synthetic construct
DADVTQTPRNRITKTGKRIMLECSQTKGHDRMYWYRQDPGLGLRLIYYSFDVKDINKGEISDGYSVSRQAQAKFSLSLESAIPNQTALYFCATSDL

SEQ ID NO: 3511 - protein, 95 aa, synthetic construct
EADIYQTPRYLVIGTGKKITLECSQTMGHDKMYWYQQDPGMELHLIHYSYGVNSTEKGDLSSESTVSRIRTEHFPLTLESARPSHTSQYLCASSE

SEQ ID NO: 3512 - protein, 94 aa, synthetic construct
DAVVTQFPRHRIIGTGKEFILQCSQNMNHVTMYWYRQDPGLGLKLVYYSPGTGSTEKGDISEGYHVSNTIASFPLTLKSASTNQTSVYLYASSS

SEQ ID NO: 3513 - protein, 95 aa, synthetic construct
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSL

SEQ ID NO: 3514 - protein, 95 aa, synthetic construct
DVKVTQSSRYLVKRTGEKVFLECVQDMDHENMFWYRQDPGLGLRLIYFSYDVKMKEKGDIPEGYSVSREKKERFSLILESASTNQTSMYLCASSL

SEQ ID NO: 3515 - protein, 96 aa, synthetic construct
SAVISQKPSRDICQRGTSLTIQCQVDSQVTMMFWYRQQPGQSLTLIATANQGSEATYESGFVIDKFPISRPNLTFSTLTVSNMSPEDSSIYLCSVE

SEQ ID NO: 3516 - protein, 94 aa, synthetic construct
SQTIHQWPATLVQPVGSPLSLECTVEGTSNPNLYWYRQAAGRGLQLLFYSVGIGQISSEVPQNLSASRPQDRQFILSSKKLLLSDSGFYLCAWS

SEQ ID NO: 3517-3639 - skipped (123 entries)

SEQ ID NO: 3640 - protein, 10 aa, synthetic construct
GFNIKSAYMH

SEQ ID NO: 3641 - protein, 11 aa, synthetic construct
SESVDSYGNSF

SEQ ID NO: 3642 - protein, 243 aa, synthetic construct
EVQLVESGGGLVQPGGSLRLSCAASGFTFSNFGMHWVRQAPGKGLEWVSYISSGSSTIYYADTLKGRFTISRDNSKNTLYLQMNSLRAEDTAVYYCARRGEGAMDYWGQGTTVTVSSGGGGSGGGGSGGGGSGGGGSDNQLTQSPSFLSASVGDRVTITCRASSSVNYIYWYQQKPGKAPKLLIYYTSNLAPGVPSRFSGSGSGNEYTLTISSLQPEDFATYYCQQFTSSPFTFGQGTKLEIK

SEQ ID NO: 3643 - protein, 16 aa, synthetic construct
GGGGSGGGGSGGGGGS

SEQ ID NO: 3644 - protein, 107 aa, synthetic construct [sequence omitted]

SEQ ID NO: 3645 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3646 - protein, 334 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKEPEA

SEQ ID NO: 3647 - protein, 330 aa, synthetic construct; identical to SEQ ID NO: 41

SEQ ID NO: 3648 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3649 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3650 - protein, 10 aa, synthetic construct
GHDFRLTYIH

SEQ ID NO: 3651 - protein, 17 aa, synthetic construct
RVSAGSGNVKYNEKFKG

SEQ ID NO: 3652 - protein, 11 aa, synthetic construct
KASQNVGNRVV

SEQ ID NO: 3653 - protein, 7 aa, synthetic construct
SSSHRYK

SEQ ID NO: 3654 - protein, 17 aa, synthetic construct
RIYAGSGNVKYNEKFKG

SEQ ID NO: 3655 - protein, 11 aa, synthetic construct
KASQNVADRVV

SEQ ID NO: 3656 - protein, 10 aa, synthetic construct
GHDFKLTYIH

SEQ ID NO: 3657 - protein, 17 aa, synthetic construct
RVSAGSGNTKYNEKFKG

SEQ ID NO: 3658 - protein, 11 aa, synthetic construct
KASQNVGDRVA

SEQ ID NO: 3659 - protein, 17 aa, synthetic construct
RVYAGSGNTKYNEKFKG

SEQ ID NO: 3660 - protein, 11 aa, synthetic construct
KASQNVEDRVV

SEQ ID NO: 3661 - protein, 17 aa, synthetic construct
RISAGSGNTKYNEKFKG

SEQ ID NO: 3662 - protein, 11 aa, synthetic construct
KASQNVGDRVV

SEQ ID NO: 3663 - protein, 17 aa, synthetic construct
RIYAGSGNTKYNEKFKG

SEQ ID NO: 3664 - protein, 9 aa, synthetic construct
LQHNSYPLT

SEQ ID NO: 3665 - protein, 17 aa, synthetic construct
RVSAGSGNVNYAQKFQG

SEQ ID NO: 3666 - protein, 11 aa, synthetic construct
RASQNVDNRLG

SEQ ID NO: 3667 - protein, 11 aa, synthetic construct
KASQNVDNRVA

SEQ ID NO: 3668 - protein, 7 aa, synthetic construct
AASSLQK

SEQ ID NO: 3669 - protein, 102 aa, synthetic construct
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSQGPFQPQH